(19) United States
(12) Patent Application Publication
(10) Pub. No.: US 2007/0134261 A1
(43) Pub. Date: Jun. 14, 2007

(54) Effectors of innate immunity

(76) Inventors: Robert E.W. Hancock, Vancouver (CA); B. Brett Finlay, Richmond (CA); Monisha Gough Scott, Portland, OR (US); Dawn Bowdish, Marston (GB); Carrie Melissa Rosenberger, Seattle, WA (US); Jon-Paul Steven Powers, Vancouver (CA); Jie Yu, Vancouver (CA); Neeloffer Mookherjee, Vancouver (CA)

Correspondence Address: DLA PIPER US LLP, 4365 EXECUTIVE DRIVE, SUITE 1100, SAN DIEGO, CA, 92121-2133, US

(21) Appl. No.: 11/400,411

(22) Filed: Apr. 7, 2006

Related U.S. Application Data
(63) Continuation-in-part of application No. 11/241,882, filed on Sep. 29, 2005.
Application No. 11/241,882 is a continuation-in-part of application No. 10/661,471, filed on Sep. 12, 2003.
Application No. 10/661,471 is a continuation-in-part of application No. 10/308,905, filed on Dec. 2, 2002.
(60) Provisional application No. 60/336,632, filed on Dec. 3, 2001.

(51) Int. Cl. A61K 39/02; C12Q 1/68

(52) U.S. Cl. 424/185.1; 435/6; 424/234.1

(57) ABSTRACT

The present invention provides a method of identifying agents that enhance innate immunity in a subject. The invention further provides a method of selectively supressing sepsis by suppressing expression of a proinflammatory gene while maintaining expression of an anti-inflammatory gene. Also provided are methods of identifying a polynucleotide or pattern of polynucleotides regulated by one or more sepsis or inflammatory inducing agents and inhibited by a peptide is described, methods of identifying a pattern of polynucleotide expression for inhibition of an inflammatory or septic response, and compounds and agents identified by the methods of the invention.

### RELATED APPLICATION DATA

This application claims priority under 35 U.S.C. §120 to U.S. patent application Ser. No. 11/241,882, filed Sep. 29, 2005, which is a continuation-in-part of U.S. patent application Ser. No. 10/661,471, filed Sep. 12, 2003, which is a continuation-in-part of U.S. patent application Ser. No. 10/308,905, filed Dec. 2, 2002, which claims priority under 35 U.S.C. §119(e) to U.S. patent application Ser. No. 60/336,632, filed Dec. 3, 2001, herein incorporated by reference in their entirety.

### FIELD OF THE INVENTION

The present invention relates generally to peptides and specifically to peptides effective as therapeutics and for drug discovery related to pathologies resulting from microbial infections and for modulating innate immunity or inflammation.

### BACKGROUND OF THE INVENTION

Infectious diseases are the leading cause of death worldwide. According to a 1999 World Health Organization study, over 13 million people die from infectious diseases each year. Infectious diseases are the third leading cause of death in North America, accounting for 20% of deaths annually and increasing by 50% since 1980. The success of many medical and surgical treatments also hinges on the control of infectious diseases. The discovery and use of antibiotics has been one of the great achievements of modem medicine. Without antibiotics, physicians would be unable to perform complex surgery, chemotherapy or most medical interventions such as catheterization.

Current sales of antibiotics are US$26 billion worldwide. However, the overuse and sometimes unwarranted use of antibiotics have resulted in the evolution of new antibiotic-resistant strains of bacteria. Antibiotic resistance has become part of the medical landscape. Bacteria such as vancomycin-resistant Enterococcus (VRE), and methicillin-resistant Staphylococcus aureus (MRSA) strains cannot be treated with antibiotics and often, patients suffering from infections with such bacteria die. Antibiotic discovery has proven to he one of the most difficult areas for new drug development and many large pharmaceutical companies have cut back or completely halted their antibiotic development programs. However, with the dramatic rise of antibiotic resistance, including the emergence of untreatable infections, there is a clear unmet medical need for novel types of anti-microbial therapies, and agents that impact on innate immunity would be one such class of agents.

The innate immune system is a highly effective and evolved general defense system. Elements of innate immunity are always present at low levels and are activated very rapidly when stimulated. Stimulation can include interaction of bacterial signaling molecules with pattern recognition receptors on the surface of the body's cells or other mechanisms of disease. Every day, humans are exposed to tens of thousands of potential pathogenic microorganisms through the food and water we ingest, the air we breathe and the surfaces, pets and people that we touch. The innate immune system acts to prevent these pathogens from causing disease. The innate immune system differs from so-called adaptive immunity (which includes antibodies and antigen-specific B- and T-lymphocytes) because it is always present, effective immediately, and relatively non-specific for any given pathogen. The adaptive immune system requires amplification of specific recognition elements and thus takes days to weeks to respond. Even when adaptive immunity is pre-stimulated by vaccination, it may take three days or more to respond to a pathogen whereas innate immunity is immediately or rapidly (hours) available. Innate immunity involves a variety of effector functions including phagocytic cells, complement, etc, but is generally incompletely understood. Generally speaking many known innate immune responses are “triggered” by the binding of microbial signaling molecules with pattern recognition receptors such as Toll-like receptors (TLR) on the surface of host cells. We now know that Toll/Interleukin-1 Receptor (TIR) domain-containing proteins play a pivotal role in initiating aspects of the inflammatory responses. Many of these effector functions are grouped together in the inflammatory response. However, too severe an inflammatory response can result in responses that are harmful to the body, and, in an extreme case, sepsis and potentially death can occur. Thus, a therapeutic intervention to boost innate immunity, which is based on stimulation of TLR signaling (for example using a TLR agonist), has the potential disadvantage that it could stimulate a potentially harmful inflammatory response and/or exacerbate the natural inflammatory response to infection.

Early responses to infection, collectively termed innate immunity and/or acute inflammation, are substantially orchestrated by various mechanisms, for example, the interaction of bacterial molecules with TLR. It has been shown that a breakdown in the appropriate regulation of the TLR pathway can cause common chronic inflammatory diseases including inflammatory bowel disease (IBD), cardiovascular disease, arthritis, and chronic interstitial nephritis. Further, TLR engagement by conserved microbial molecules results in the translocation of the pivotal transcription factor NFκB and the transcription of ‘early-response’ genes encoding, for example, cytokines, chemokines, selected antimicrobial/host defense peptides, acute phase proteins, cell adhesion molecules, co-stimulatory molecules and proteins required for negative feedback to suppress these responses. Alternatively, an exaggerated response to bacterial stimuli underlies a clinical condition called Systemic Inflammatory Response Syndrome, or sepsis, in which high levels of cytokines and inflammatory mediators become destructive, causing organ failure, cardiovascular shock and/or death.

Sepsis occurs in approximately 780,000 patients in North America annually. Sepsis may develop as a result of infections acquired in the community such as pneumonia, or it may be a complication of the treatment of trauma, cancer or major surgery. Severe sepsis occurs when the body is overwhelmed by the inflammatory response and body organs begin to fail. Up to 120,000 deaths occur annually in the United Stated due to sepsis. Sepsis may also involve pathogenic microorganisms or toxins in the blood (e.g., septicemia), which is a leading cause of death among humans. Gram-negative bacteria are the organisms most commonly associated with such diseases. However, gram-positive bacteria are an increasing cause of infections. Gram-negative and Gram-positive bacteria and their components can all cause sepsis.

The presence of microbial components induces the release of pro-inflammatory cytokines of which tumor necrosis factor-α (TNF-α) is of extreme importance. TNF-α and other pro-inflammatory cytokines can then cause the release of other pro-inflammatory mediators and lead to an inflammatory cascade. Gram-negative sepsis is usually caused by the release of the bacterial outer membrane component, lipopolysaccharide (LPS; also referred to as endotoxin). Endotoxin in the blood, called endotoxemia comes primarily from a bacterial infection, and may be released during treatment with antibiotics. Gram-positive sepsis can be caused by the release of bacterial cell wall components such as lipoteichoic acid (LTA), peptidoglycan (PG)i rhamnose-glucose polymers made by Streptococci, or capsular polysaccharides made by Staphylococci. Bacterial or other non-mammalian DNA that, unlike mammalian DNA, frequently contains unmethylated cytosine-guanosine dimers (CpG DNA) has also been shown to induce septic conditions including the production of TNF-α. Mammalian DNA contains CpG dinucleotides at a much lower frequency, often in a methylated form. In addition to their natural release during bacterial infections, antibiotic treatment can also cause release of the bacterial cell wall components LPS and LTA and probably also bacterial DNA. This can then hinder recovery from infection or even cause sepsis.

In humans, inhalation of the Gram-negative bacterial component lipopolysaccharide (LPS), a TLR4 ligand, results in increased cytokine and chemokine (TNFα, IL1β, IL6, IL8) mRNA and protein expression within 4-6 hr of inhalation. In mutant mice lacking responsiveness to LPS animals do not develop septic shock, demonstrating that the response to endotoxin is sufficient to promote sepsis. Other TLRs exist in humans and can be engaged by other pathogen molecules to drive septic responses. For example, TLR2 is engaged by the signature cell wall-associated molecule lipoteichoic acid (LTA) from Gram positive bacteria, while DNA containing the signature dinucleotide pair unmethylated CpG engages TLR9 and can also stimulate proinflammatory Cytokine production. The nature, duration and intensity of inflammatory/septic responses are considered to involve the interplay between TLR and other receptors, different adaptor molecules such as MyD88, TIRAP/Mal and TRIF, and different signaling pathways. An ideal therapeutic regulator of the inflammatory response would be antagonistic to potentially lethal conditions such as septic shock by interacting with inflammatory signaling pathways but maintain innate immune defenses against bacterial infections, thus sustaining a balance between the protective and destructive components of inflammation.

Cationic host defense peptides (also known as antimicrobial peptides) are crucial molecules in host defense against pathogenic microbe challenge. These peptides have been demonstrated to have a wide range of functions ranging from direct antimicrobial activity to a broad range of immunomodulatory functions. They are widely distributed in nature, existing in organisms from insects to plants to mammals. The family includes defensins, cathelicidins, and histatins. Cathelicidins are small (12 to around 50 amino acids) cationic peptides and are amphipathic in nature with ˜50% hydrophobic residues. Mammalian cathelicidins are synthesized in a precursor pro-form that requires (generally-extracellular) proteolytic processing to generate the mature peptide. The only endogenous cathelicidin in humans is hCAP-18 (SEQ ID NO: 1) which is found at high concentrations in its unprocessed form (hCAP-18) in the granules of neutrophils and is processed upon degranulation and release. It is also produced by epithelial cells and keratinocytes, etc., as the hCAP-18 precursor form, and is found as the processed 37-amino acid peptide SEQ ID NO: 1 in a number of tissues and bodily fluids including gastric juices, saliva, semen, sweat, plasma, airway surface liquid and breast milk.

Cationic peptides are being increasingly recognized as a form of defense against infection, and although the major effects recognized in the scientific and patent literature were the antimicrobial effects (Hancock, R. E. W., and R. Lehrer. 1998. Cationic peptides: a new source of antibiotics. Trends in Biotechnology 16: 82-88.), it is now becoming increasingly clear that they are effectors in other aspects of innate immunity (Hancock, R. E. W. and G. Diamond. 2000. The role of cationic peptides in innate host defenses. Trends in Microbiology 8:402-410.; Hancock, R. E. W. 2001. Cationic peptides: effectors in innate immunity and novel antimicrobials. Lancet Infectious Diseases 1 :156-164).

Some cationic peptides have an affinity for binding bacterial products such as LPS and LTA. Such cationic peptides can suppress cytokine production in response to LPS, and to varying extents can prevent lethal shock. However it has not been proven as to whether such effects are due to binding of the peptides to LPS and LTA, or due to a direct interaction of the peptides with host cells. Cationic peptides are induced, in response to challenge by microbes or microbial signaling molecules like LPS, by a regulatory pathway similar to that used by the mammalian immune system (involving Toll receptors and the transcription factor; NFκB). Cationic peptides therefore appear to have a key role in innate immunity. Mutations that affect the induction of antibacterial peptides can reduce survival in response to bacterial challenge. As well, mutations of the Toll pathway of Drosophila that lead to decreased antiftingal peptide expression result in increased susceptibility to lethal fungal infections. In humans, patients with specific granule deficiency syndrome, completely lacking in α-defensins, suffer from frequent and severe bacterial infections. Other evidence includes the inducibility of some peptides by infectious agents, and the very high concentrations of such peptides that have been recorded at sites of inflammation. Cationic peptides may also regulate cell migration, to promote the ability of leukocytes to combat bacterial infections. For example, two human α-defensin peptides, HNP-1 and HNP-2, have been indicated to have direct chemotactic activity for murine and human T cells and monocytes, and human β-defensins appear to act as chemoattractants for immature dendritic cells and memory T cells through interaction with CCR6. Similarly, the porcine cationic peptide PR-39 was found to be chemotactic for neutrophils. It is unclear however as to whether peptides of different structures and compositions share these properties.

The single known cathelicidin from humans, SEQ ID NO: 1, is produced by myeloid precursors, testis, and human keratinocytes during inflammatory disorders and airway epithelium. The characteristic feature of cathelicidin peptides is a high level of sequence identity at the N-terminus prepro regions termed the cathelin domain. Cathelicidin peptides are stored as inactive propeptide precursors that, upon stimulation, are processed into active peptides.

### SUMMARY OF THE INVENTION

The present invention is based on the seminal discovery that based on patterns of polynucleotide expression regulated by endotoxic lipopolysaccharide, lipoteichoic acid, CpG DNA, or other cellular components (e.g., microbe or their cellular components), and affected by cationic peptides, one can screen for novel compounds that block or reduce sepsis and/or inflammation in a subject. Further, based on the use of cationic peptides as a tool, one can identify selective enhancers of innate immunity that do not trigger the sepsis reaction and that can block/dampen inflammatory and/or septic responses.

Thus, in one embodiment, a method of identifying a polynucleotide or pattern of polynucleotides regulated by one or more sepsis or inflammatory inducing agents and inhibited by a cationic peptide, is provided. The method of the invention includes contacting cells containing polynucleotide or polynucleotides with one or more sepsis or inflammatory inducing agents and contacting the cells containing polynucleotide or polynucleotides with a cationic peptide either simultaneously or immediately thereafter. Differences in expression are detected in the presence and absence of the cationic peptide, and a change in expression, either up- or down-regulation, is indicative of a polynucleotide or pattern of polynucleotides that is regulated by a sepsis or inflammatory inducing agent and inhibited by a cationic peptide. In another aspect the invention provides a polynucleotide or polynucleotides identified by the above method. Examples of sepsis or inflammatory regulatory agents include LPS, LTA or CpG DNA or microbial components (or any combination thereof), or related agents.

In another embodiment, the invention provides a method of identi fying an agent that blocks sepsis or inflammation including combining a polynucleotide identified by the method set forth above with an agent wherein expression of the polynucleotide in the presence of the agent is modulated as compared with expression in the absence of the agent and wherein the modulation in expression affects an inflammatory or septic response.

In another embodiment, the invention provides a method of identifying a pattern of polynucleotide expression for inhibition of an inflammatory or septic response by 1) contacting cells with LPS, LTA and/or CpG DNA in the presence or absence of a cationic peptide and 2) detecting a pattern of polynucleotide expression for the cells in the presence and absence of the peptide. The pattern obtained in the presence of the peptide represents inhibition of an inflammatory or septic response. In another aspect the pattern obtained in the presence of the peptide is compared to the pattern of a test compound to identify a compound that provides a. similar pattern. In another aspect the invention provides a compound identified by the foregoing method.

In another embodiment, the invention provides a method of identifying an agent that selectively enhances innate immunity by contacting cells containing a polynucleotide or polynucleotides that encode a polypeptide involved in innate immunity, with an agent of interest, wherein expression of the polynucleotide in the presence of the agent is modulated as compared with expression of the polynucleotide in the absence of the agent and wherein the modulated expression results in enhancement of innate immunity. Preferably, the agent does not stimulate a sepsis reaction in a subject. In one aspect, the agent increases the expression of an anti-inflammatory polynucleotide. Exemplary, but non-limiting anti-inflammatory polynucleotides encode proteins such as IL-1 R antagonist homolog 1 (AI167887), IL-10 R beta (AA486393), IL-10 R alpha (U00672) TNF Receptor member 1B (AA150416), TNF receptor member 5 (H98636), TNF receptor member 11b (AA194983), IK cytokine down-regulator of HLA II (R39227), TGF-B inducible early growth response 2 (AI473938), CD2 (AA927710), IL-19 (NM_{—}013371) or IL-10 (M57627). In one aspect, the agent decreases the expression of polynucleotides encoding proteasome subunits involved in NF-κB activation such as proteasome subunit 26S (D78151). In one aspect, the agent may act as an antagonist of protein kinases. In one aspect, the agent is a peptide selected from SEQ ID NO:4-54.

In another embodiment, the invention provides a method of identifying an agent that selectively suppresses the proinflammatory response of cells containing a polynucleotide or polynucleotides that encode a polypeptide involved in innate immunity. The method includes contacting the cells with microbes, or the TLR ligands and agonists derived from those microbes, and further contacting the cells with an agent of interest, wherein the agent decreases the expression of a proinflammatory gene encoding the polynucleotide as compared with expression of the proinflammatory gene in the absence of the agent. In one aspect, the modulated expression results in suppression of proinflammatory and septic responses. Preferably, the agent does not stimulate a sepsis reaction in a subject. Exemplary, but non-limiting proinflammatory genes include TNFα, TNFAIP2, IL-1β. IL-6, NFKB1 and RELA.

In another embodiment, the invention provides a method of identifying an agent that enhances innate immunity by contacting cells containing a polynucleotide or polynucleotides that encode a polypeptide involved in innate immunity, with an agent of interest, wherein the agent suppresses inflammation and sepsis while increasing the expression of an anti-inflammatory gene encoding the polynucleotide as compared with expression of the anti-inflammatory gene in the absence of the agent and wherein the modulated expression results in enhancement of innate immunity. In one aspect, the agent inhibits the expression of proinflammatory molecules such as TNFα, IL1-β, IL-6, TNFα, TNFAIP2, or the p50 or p65 subunits of transcription factor NFκB. In another aspect, inflammation is induced by a microbe or a microbial ligand acting on a Toll-like receptor such as Toll-like receptor-2, Toll-like receptor-4, or Toll-like receptor-9. Microbial ligands include, but are not limited to a bacterial endotoxin, lipopolysaccharide, lipoteichoic acid or CpG DNA. Exemplary, but non-limiting anti-inflammatory genes include ZNF83, NFKBIA, Q9P188, INVS, DIAPHI, IER3, Q9H640, GBP2, NANS, Q86XN7, Q9H9M1, TNFAIP3, Q96MJ8, Q9BSE2, Q9H753, NTNG1, INHBE, BCL6, CXCL1, EHD1, RELB, HRK, CCL4, SESN2, NAB1, EBI3, DDX21, XBP1, SLURP1, ARS, HDAC10, MEP1A, RAP2C, GYS1, RARRES3, PPY, NFKB1, MTL4_HUMAN, Q9H040, and Q9NUP6.

In another embodiment, the invention provides a method of identifying an agent that is capable of selectively enhancing innate immunity by contacting cells containing one or more genes that encode a polypeptide involved in innate immunity and protection against an infection, with an agent of interest, wherein expression of the one or more genes in the presence of the agent is modulated as compared with expression of the one or more genes in the absence of the agent, and wherein the modulated expression results in enhancement of innate immunity. In one aspect, the invention includes agents identified by the methods. In another aspect, the agent does not stimulate a septic reaction, but does stimulate expression of the one or more genes. Exemplary, but non-limiting genes include any of the genes listed in Table 69. In one embodiment, the one or more genes encode G-coupled protein receptors that initiate signaling from extracellular ligands. Exemplary, but non-limiting genes encoding G-coupled protein receptors that initiate signaling from extracellular ligands include GPR55, GPR6, GPR30, GPCR42, CASR, and EDG2. In another embodiment, the one or more genes encode chemokines or interleukins that attract imrnune cells. Exemplary, but non-limiting genes encoding chemokines or interleukins that attract immune cells include MCP-1, MCP-3, IL-8, CXCL-1, IL-17C, and IL-19. In another embodiment, the one or more genes encode receptors for chemokines. An exemplary, but non-limiting gene encoding a receptor for chemokines includes CCR7. In another embodiment, the one or more genes encode transcription factors that mediate selective gene expression. Exemplary, but non-limiting genes encoding transcription factors that mediate selective gene expression include JAK1, STAT1, ELF1, Q9Y4C1, ETV4, POU1F1, ZNF254, ZNF292, ZNF78L1, HOXD3, and DLX5. In another embodiment, the one or more genes encode tyrosine-protein kinase or tyrosine-protein kinase receptors. Exemplary, but non-limiting genes encoding tyrosine-protein kinase or tyrosine-protein kinase receptors include MAP2K6, NTRK3, PLCG1, EFNA2, and NCK1. In another embodiment, the one or more genes encode adhesion molecules that mediate cell attachment and interaction. Exemplary, but non-limiting adhesion molecules that mediate cell attachment and interaction include the ICAM, NCAM families, and PTPRF. Exemplary, but non-limiting genes encoding adhesion molecules that mediate cell attachment and interaction include ICAM3, NCAM2, and PTPRF. In another embodiment, the one or more genes are involved in actin polymerization or cytoskeletal remodeling. Exemplary, but non-limiting genes involved in actin polymerization or cytoskeletal remodeling include Integrin-α, EPHA4, ARHGAP6, and DST. In another embodiment, the one or more genes encode regulators of transcription factors. Exemplary, but non-limiting genes encoding regulators of transcription factors include TRIP4, GMEB2, GSK3B, ARNT, BACH, ARID3A, HIPK2, POLR2D, TGIF, SSBP3, and FYB. In another embodiment, the one or more genes encode transmembrane receptors and adapters of signaling pathways. Exemplary, but non-limiting genes encoding transmembrane receptors and adapters of signaling pathways include WNT5B, FZD10, TIRAP, and REPS1. In another embodiment, the one or more genes encode proteins involved in antiviral activity. Exemplary, but non-limiting genes encoding proteins involved in antiviral activity include IFNA2, STAT1, MNDA, and IFNA2. In another embodiment, the agent stimulates the JAK-STAT pathway. In another embodiment, the agent stimulates expression of one or more genes selected from the group consisting of JAK2, STAT1, STAT3, SOCS1, and. IL-19. In another embodiment, the agent stimulates the P13K pathway. In another embodiment, the agent stimulates expression of one or more genes selected from the group consisting of BACH2/PIK3CB, Akt, CREB, IL-6, and MCP-3. In another embodiment, the agent stimulates the ERK1/2 mitogen activated kinase pathway. In another embodiment, the agent stimulates expression of one or more genes selected from the group consisting of MAP3K1 and PP2A. In another embodiment, the agent stimulates the p38 mitogen activated kinase pathway. In another embodiment, the agent stimulates expression of one or more genes selected from the group consisting of MINK1/MAP4K6, MAP2K6, and MAP2K4. In another embodiment, the agent transiently stimulates the NFκB pathway. In another embodiment, the agent stimulates expression of one or more genes selected from the group consisting of TIRAP, NFκB2 (p52), DUSP14, ICAM3, TRIP4, MMP17, ITGB4, ZNF36, ZNF251, BNIP1, CD226, NRXN1, and TNC. In another embodiment, the agent stimulates the AP-1, JNK or Wnt pathways. In another embodiment, the agent stimulates expression of one or more genes selected from the group consisting of TRIP4, TIRAP, HIPK2, GSK3B, and FZD10.

In another embodiment, the invention provides a method of identifying a pattern of gene expression for identification of an agent that selectively enhances innate immunity by contacting a cell containing one or more genes that encode a polypeptide involved in innate immunity and defense against infections, with an agent of interest, wherein expression of the one or more genes in the presence of the agent is modulated as compared with expression of the one or more genes in the absence of the agent, and wherein the modulated expression results in enhancement of innate immunity. In one embodiment, the modulated expression is a marker of enhancement of innate immunity. In another embodiment, the method further includes determining the efficacy of compounds that enhance innate immunity. In another embodiment, the one or more genes are any gene shown in Table 69. In another embodiment, the one or more genes express IL-8, IL-6, IL-19, CXCL-1, MCP-3, or MCP-1. In another embodiment, the modulated expression occurs in the presence of a bacterial signature molecule. The bacterial signature molecule may be a Toll-like receptor agonist such as bacterial lipopolysaccharide, lipoteichoic acid, and CpG bacterial signature DNA. In another embodiment, the one or more genes are any gene shown in Table 71.

In another embodiment, the invention provides a method of identifying an agent that is capable of selectively enhancing innate immunity in the presence of an infection or bacterial signature molecule by contacting a cell containing one or more genes that encode a polypeptide involved in innate immunity, with an agent of interest in the presence of a bacterial signature molecule, wherein expression of the one or more genes in the presence of the agent and bacterial signature molecule is modulated as compared with expression of the one or more genes in the absence of the agent and bacterial signature molecule, and wherein the modulated expression results in enhancement of innate immunity. In one aspect, the invention includes agents identified by the methods. In another aspect, the bacterial signature molecule is a Toll-like receptor agonist such as bacterial lipopolysaccharide, lipoteichoic acid, and CpG bacterial signature DNA. In another embodiment, the one or genes are any gene shown in Table 71. In another embodiment, the agent does not stimulate a septic reaction. In another embodiment, the agent has anti-endotoxic activity. In another embodiment, the one ore more genes are selected from the group consisting of GPD1, Q8NI35, FEZ2, NRXN1, PLCG1, Q7RTU0, ALDOB, Q9H5P1, SYT11, UBXD2, PROZ, PLAC8, Q96PN6, ASTN2, O60290, FTCD, NFKB2, CTLA4, PSMA1, CCL2, HNF4A, MAFF, FBXO32, TNFα, NPAS2, ICAM3, Q8NC30, Q81UC6, O94940, CGI-117, KDELR1, IFITM1 and COL7A1. In another embodiment, the agent stimulates transient IκBα degradation or transient NFκB subunit p50 translocation. In another embodiment, the method further includes contacting the cell with IL-1β. In another embodiment, the one or more genes encode chemokines. Exemplary, but non-limiting genes that encode chemokines include CCL20, CCL23, IL-6, and MCP-3. In another embodiment, the one or more genes encode cytokine receptors. Exemplary, but non-limiting genes that encode chemokines include EBI3 and IL7R. In another embodiment, the one or more genes encode factors involved in lymphocyte activation. Exemplary, but non-limiting genes that encode factors involved in lymphocyte activation include SLAMF1, CD58, and IL32. In another embodiment, the one or more genes encode regulators of signal transduction. Exemplary, but non-limiting genes that encode regulators of signal transduction include MAP2K2, DUSP5, MAPK8IP3, RIN2, RANBP9, IP3 3-kinase A, BATF, IRAK3, NM1, SP3, RAP2C, PNRC1, NEK1, CHC1, ZNF219, ZNF593, WIF1, PIM2, CD79A, and LATS2. In another embodiment, the one or more genes encode substrate transporters. Exemplary, but non-limiting genes that encode substrate transporters include SLC23A3 and SLC17A5. In another embodiment, the one or more genes encode apoptosis regulators. Exemplary, but non-limiting genes that encode apoptosis regulators include BOK, BIRC3, TNFRSF6, and CASP9. In another embodiment, the one or more genes encode genes associated with plasma membrane. Exemplary, but non-limiting genes that encode genes associated with plasma membrane include STIM1, BPAG1, PTPN4, TRIM36, SDK1, and FNDC5. In another embodiment, the one or more genes encode genes involved in selective ion transport and in mediating selective ion-channels. Exemplary, but non-limiting genes that encode genes involved in selective ion transport and in mediating selective ion-channels include VGCNL1, TRPC5, CACNA1B, KCNA6, KCNJ2, KCNA10, and AQP9. In another embodiment, the one or more genes encode growth modulating genes or genes involved in wound healing. xemplary, but non-limiting genes that encode growth modulating genes or genes involved in wound healing include FGF10 and AREG. In another embodiment, the one or more genes encode inflammatory mediators. Exemplary, but non-limiting genes that encode inflammatory mediators include PTGS2, SOD2, TNFAIP8, and TNIP3. In another embodiment, the method further includes contacting the cell with IL-1β, wherein the agent stimulates the PI3 kinase pathway. In another embodiment, the agent stimulates transient IκBα phosphorylation and p50 nuclear translocation. In another embodiment, the one or more genes encodes a G-protein coupled receptor or a purinergic receptor. An exemplary, but non-limiting purinergic receptor is P2X7. In another embodiment, the agent fturther stimulates phosphorylation of Akt, which stimulates activation of CREB.

In another embodiment, the invention provides a method of identifying an agent that selectively reduces inflammation by contacting a cell containing one or more genes that encode a polypeptide involved in sepsis, with an agent of interest, wherein the agent reduces expression of the one or more genes compared with expression of the one or more genes in the absence of the agent. In another embodiment, the one or more genes are selected from the group consisting of GPD1, Q8NI35, FEZ2, NRXN, PLCG1, Q7RTU0, ALDOB, Q9H5P1, SYT11, UBXD2, PROZ, PLAC8, Q96PN6, ASTN2, O60290, FTCD, NFKB2, CTLA4, PSMA1, CCL2, HNF4A, MAFF, FBXO32, TNF, NPAS2, ICAM3, Q8NC30, Q81UC6, O94940, CGI-117, KDELR1, IFITM1, and COL7A1.

In another embodiment, the invention provides a method of identifying an agent that selectively suppresses sepsis by contacting cells containing a polynucleotide or polynucleotides that encode a polypeptide involved in innate immunity, with an agent of interest, wherein the agent suppresses expression of a proinflammatory gene while maintaining expression of an anti-inflammatory gene encoding the polynucleotide as compared with expression of the anti-inflammatory gene in the absence of the agent. In one aspect, the agent inhibits the expression of proinflammatory molecules such as TNFα, IL1-β, IL-6, TNFα, TNFAIP2, or the p50 or p65 subunits of transcription factor NFκB. In another aspect, inflammation is induced by a microbe or a microbial ligand acting on a Toll-like receptor such as Toll-like receptor-2, Toll-like receptor-4, or Toll-like receptor-9. Microbial ligands include, but are not limited to a bacterial endotoxin, lipopolysaccharide, lipoteichoic acid or CpG DNA. Exemplary, but non-limiting anti-inflammatory genes include ZNF83, NFKBIA, Q9P188, INVS, DIAPH1, IER3, Q9H640, GBP2, NANS, Q86XN7, Q9H9M1, TNFAIP3, Q96MJ8, Q9BSE2, Q9H753, NTNG1, INHBE, BCL6, CXCL1, EHD1, RELB, HRK, CCL4, SESN2, NAB1, EBI3, DDX21, XBP1, SLURP1, ARS, HDAC10, MEP1A, RAP2C, GYS1, RARRES3, PPY, NFKB1, MTL4_HUMAN, Q9H040, and Q9NUP6. Exemplary, but non-limiting proinflammatory genes include LC2A6, SLC4A5, MCL1, Q86XN7, Q9H9M1, Q86UU3, Q8NAA1, C15orf2, TNFRSF5, FACL6, Q8IW99, Q96AU7, PRB4, Q9NWP0, Q8NF24, Q8TEE5, PDE4DIP, NUDT4, DUSP2, LMAN2, RELB, SNF1LK, TNFα, GHRHR, TNFSF6, ENSG00000181873, IRAK2, CKB, CASR, KRTAP4-10, ARHGEF3, CYP3A4, CYP3A7, GPR27, PAX8, GAP43, Q96M75, Q9H568, AGTRL1, C1orf22, EHD1, ADRA1B, SSTR2, SYNE1, ENSG00000139977, PTPRK, O15059, Q9NZ16, N4BP3, KIAA0341, Q8IVT2, Q9NV39, HIP1R, HIP12, KIAA0655, IL-6, TNFAIP2, RCV1, FBLN2, TWIST2, PARD6B, DCK, TULP4, LK10, SPAP1, IBRDC2, JAM2, NRG2, CBARA1, DLG2, PRKCBP1, MGLL, Q9BYE1, MARCKS, Q96N98, Q8NBY1, Q96AF2, Q9BS16, PPP2CA, RAB38, VCAM1, TTTY8, HTR2A, SERPINB10, O75121, Q9BVE1, ZCCHC2, CXCL2, GADD45B, KARS, SCG2, SLC17A2, FLT4, Q9NXT0, Q96L19, BICD1, HCK, Q8N9T8, Q9H978, PPP1R1A, PAX7, EBI3, THRA, SLC16A10, INPP5E, Q9H967, NFKB1, MKL1, SS18L2, TNFRSF9, TNFAIP6, Q9Y2K2, ING5, IL1A, TMH, HDAC4, KPTN, SEC61G, Q9Y484, FRAS1, IER5, Q8N137, Q8NCB8, Q96HQ0, Q9H5P0, TXNRD1, CAV2, SCARB1, MAP3K5, PDHX, TCEB3, C21orf55, MPHOSPH10, PDE8A, TFR2, FARP1, SERPINA1, MYO15A, RABGGTA, KCNMB4, Q9BR02, APOB, MYC, FARP2, TFAP2BL1, Q86U90, Q9H5F8, USH1C, IL-8, SOX2, Q9NVC3, NEIL2, TNIP1, ADRA1D, PCDHB9, Q12987, TNFRSF6, C20orf72, DNAJA3, MAB21 L1, BIRC2, MYST1, CNN3, CXCL3, CD80, CSRP2, RAD51L1, ADARB1, TNFSF8, Q8IW74, UXS1, ENSG00000182364, TNFRSF7, MYBL2, RAB33A, ATIC, CAMK1, CCNT1, KCNE4, BOK, NF2, PDP2, and KIAA1348.

In another embodiment, the invention provides a method of identifying an agent that selectively suppresses sepsis by contacting cells containing a polynucleotide or polynucleotides that encode a polypeptide involved in innate immunity, with an agent of interest, wherein the agent induces signaling of the JAK-STAT pathway and suppresses expression of a proinflammatory gene while maintaining expression of an anti-inflammatory gene encoding the polynucleotide as compared with expression of the anti-inflammatory gene in the absence of the agent. In one aspect, the agent inhibits the expression of proinflammatory molecules such as TNFα, NFκB2, IL1-β, IL-6, IL-8, CXCL-1, TNFAIP2, or the p50 or p65 subunits of transcription factor NFκB. In another aspect, inflammation is induced by a microbe or a microbial ligand acting on a Toll-like receptor such as Toll-like receptor-2, Toll-like receptor-4, or Toll-like receptor-9. Microbial ligands include, but are not limited to a bacterial endotoxin, lipopolysaccharide, lipoteichoic acid or CpG DNA. Exemplary, but non-limiting anti-inflammatory genes include one or more genes listed in Table 69. Exemplary, but non-limiting proinflammatory genes include one or more genes listed in Table 72.

In another embodiment, the invention provides a method of identifying a pattern of polynucleotide expression for identification of a compound that selectively enhances innate immunity. The invention includes detecting a pattern of polynucleotide expression for cells contacted in the presence and absence of a cationic peptide, wherein the pattern in the presence of the peptide represents stimulation of innate immunity; detecting a pattern of polynucleotide expression for cells contacted in the presence of a test compound, wherein a pattern with the test compound that is similar to the pattern observed in the presence of the cationic peptide, is indicative of a compound that enhances innate immunity.

In another embodiment, the invention provides a method for inferring a state of infection in a mammalian subject from a nucleic acid sample of the subject by identifying in the nucleic acid sample a polynucleotide expression pattern exemplified by an increase in polynucleotide expression of at least 2 polynucleotides in Table 50, 51 and or 52, as compared to a non-infected subject. Also included is a polynucleotide expression pattern obtained by any of the methods described above.

In another aspect a cationic peptide that is an antagonist of CXCR-4 is provided. In still another aspect, a method of identifying a cationic peptide that is an antagonist of CXCR-4 by contacting T cells with SDF-1 in the presence of absence of a test peptide and measuring chemotaxis is provided. A decrease in chemotaxis in the presence of the test peptide is indicative of a peptide that is an antagonist of CXCR-4. Cationic peptide also acts to reduce the expression of the SDF-1 receptor polynucleotide (NM_{—}012428).

In all of the above described methods, the compounds or agents of the invention include but are not limited to peptides, cationic peptides, peptidomimetics, chemical compounds, polypeptides, nucleic acid molecules and the like.

In still another aspect the invention provides an isolated cationic peptide. An isolated cationic peptide of the invention is represented by one of the following general formulas and the single letter amino acid code:
X₁X₂X₃IX₄PX₄IPX₅X₂X₁ (SEQ ID NO: 4), where X₁ is one or two of R, L or K, X₂ is one of C, S or A, X₃ is one of R or P, X₄ is one of A or V and X₅ is one of V or W;
X₁LX₂X₃KX₄X₂X₅X₃PX₃X₁ (SEQ ID NO: 11), where X₁ is one or two of D, E, S, T or N, X2 is one or two of P, G or D, X₃ is one of G, A, V, L, I or Y, X₄ is one of R, K or H and X₅ is one of S, T, C, M or R;
X₁X₂X₃X₄WX₄WX₄X₅K (SEQ ID NO: 18), where X₁ is one to four chosen from A, P or R, X₂ is one or two aromatic amino acids (F, Y and W), X₃ is one of P or K, X₄ is one, two or none chosen from A, P, Y or W and X₅ is one to three chosen from R or P;
X₁X₂X₃X₄X₁VX₃X₄RGX₄X₃X₄X₁X₃X₁ (SEQ ID NO: 25) where X₁ is one or two of R or K, X₂ is a polar or charged amino acid (S, T, M, N, Q, D, E, K, R and H), X₃ is C, S, M, D or A and X₄ is F, I, V, M or R;
X₁X₂X₃X₄X₁VX₅X₄RGX₄X₅X₄X₁X₃X₁ (SEQ ID NO: 32), where X₁ is one or two of R or K, X₂ is a polar or charged amino acid (S, T, M, N, Q, D, E, K, R and H), X₃ is one of C, S, M, D or A, X₄ is oneofF, I, V, M or R and X₅ is one of A, I, S, M, D or R; and
KX₁KX₂FX₂KMLMX₂ALKKX₃ (SEQ ID NO: 39), where X₁ is a polar amino acid (C, S, T, M, N and Q); X₂ is one of A, L, S or K and X₃ is 1-17 amino acids chosen from G, A, V, L, I, P, F, S, T, K and H;
KWKX₂X₁X₁X₂X₂X₁X₂X₂X₁X₁ X₂X₂IFHTALKPISS (SEQ ID NO: 46), where X₁ is a hydrophobic amino acid and X₂ is a hydrophilic amino acid.

Additionally, in another aspect the invention provides isolated cationic peptides

| | |
|---|---|
| KWKSFLRTFKSPVRTVFHTALKPISS | (SEQ ID NO: 53) |
| and | |
| KWKSYAHTIMSPVRLVFHTALKPISS. | (SEQ ID NO: 54) |

Also provided are nucleic acid sequences encoding the cationic peptides of the invention, vectors including such polynucleotides and host cells containing the vectors.

In another embodiment, the invention provides methods for stimulating or enhancing innate immunity in a subject comprising administering to the subject a peptide of the invention, for example, peptides set forth in SEQ ID NO:1-4, 11, 18, 25, 32, 39, 46, 53 or 54. As shown in the Examples herein, innate immunity can be evidenced by monocyte activation, proliferation, differentiation, or MAP kinase pathway activation just by way of example. In one aspect, the method includes further administering a serum factor such as GM-CSF to the subject. The subject is preferably any mammal and more particularly a human subject.

In another embodiment, the invention provides a method of stimulating innate immunity in a subject having or at risk of having an infection including administering to the subject a sub-optimal concentration of an antibiotic in combination with a peptide of the invention. In one aspect, the peptide is SEQ ID NO:1 or SEQ ID NO:7.

In all of the above described embodiments, the methods may be performed ex vivo.

### BRIEF DESCRIPTION OF THE FIGURES

FIG. 1 demonstrates the synergy of SEQ ID NO: 7 with cefepime in curing S. aureus infections. CD-1 mice (8/group) were given 1×10⁷ S. aureus in 5% porcine mucin via IP injection. Test compound (50 μg-2.5 mg/kg) was given via a separate IP injection 6 hours after S. aureus. At this time Cefepime was also given at a dose of 0.1 mg/kg. Mice were euthanized 24 hr later, blood removed and plated for viable counts. The average±standard error is shown. This experiment was repeated twice.

FIG. 2 shows exposure to SEQ ID NO: 1 induces phosphorylation of ERK1/2 and p38. Lysates from human peripheral blood derived monocytes were exposed to 50 μg/ml of SEQ ID NO: 1 for 15 minutes. A) Antibodies specific for the phosphorylated forms of ERK and p38 were used to detect activation of ERK1/2 and p38. All donors tested showed increased phosphorylation of ERK1/2 and p38 in response to SEQ ID NO: 1 treatment. One representative donor of eight is shown. Relative amounts of phosphorylation of ERK (B) and p38(C) were determined by dividing the intensities of the phosphorylated bands by the intensity of the corresponding control band as described in the Materials and Methods in Example 12.

FIG. 3 shows SEQ ID NO: 1 induced phosphorylation of ERK1/2 does not occur in the absence of serum and the magnitude of phosphorylation is dependent upon the type of serum present. Human blood derived monocytes were treated with 50 μg/ml of SEQ ID NO: 1 for 15 minutes. Lysates were run on a 12% acrylamide gel then transferred to nitrocellulose membrane and probed with antibodies specific for the phosphorylated (active) form of the kinase. To normalize for protein loading, the blots were reprobed with β-actin. Quantification was done with ImageJ software. The FIG. 3 insert demonstrates that SEQ ID NO: 1 is unable to induce MAPK activation in human monocytes under serum free conditions. Cells were exposed to 50 mg/ml of SEQ ID NO: 1 (+), or endotoxin free water (−) as a vehicle control, for 15 minutes. (A) After exposure to SEQ ID NO: 1 in media containing 10% fetal calf serum, phosphorylated ERK1/2 was detectable, however, no phosphorylation of ERK1/2 was detected in the absence of serum (n=3). (B) Elk-1, a transcription factor downstream of ERK1/2, was activated (phosphorylated) upon exposure to 50 μg/ml of SEQ ID NO: 1 in media containing 10% fetal calf serum, but not in the absence of serum (n=2).

FIG. 4 shows SEQ ID NO: 1 induced activation of ERK1/2 occurs at lower concentrations and is amplified in the presence of certain cytokines. When freshly isolated monocytes were stimulated in media containing both GM-CSF (100 ng/ml) and IL-4 (100 ng/ml) SEQ ID NO: 1 induced phosphorylation of ERK1/2 was apparent at concentrations as low as 5 μg/ml. This synergistic activation of ERK1/2 seems to be due primarily to GM-CSF.

FIG. 5 shows peptide affects both transcription of various cytokine genes and release of IL-8 in the 16HBE4o-human bronchial epithelial cell line. Cells were grown to confluency on a semi-permeable membrane and stimulated on the apical surface with 50 μg/ml of SEQ ID NO: 1 for four hours. A) SEQ ID NO: 1 treated cells produced significantly more IL-8 than controls, as detected by ELISA in the supernatant collected from the apical surface, but not from the basolateral surface. Mean±SE of three independent experiments shown, asterisk indicates p=0.002. B) RNA was collected from the above experiments and RT-PCR was performed. A number of cytokine genes known to be regulated by either ERK1/2 or p38 were up-regulated upon stimulation with peptide. The average of two independent experiments is shown.

FIG. 6 is a graphical representation showing that SEQ ID NO: 1 suppresses LPS-induced secretion of TNF-α. The concentration of the pro-inflammatory cytokine TNFα (Y-axis) was monitored in the tissue culture supernatant or cytoplasmic extracts of cells by ELISA. The results are an average (±standard deviation) of three independent experiments. (A) THP-1 cells were stimulated with 10 ng/ml (-●-) or 100 ng/ml (-▪-) of LPS in the presence of increasing concentrations of SEQ ID NO: 1 (X-axis) for 4 hr. (B) PBMCs were stimulated with 100 ng/ml of LPS in presence or absence of 20 μg/ml SEQ ID NO: 1 for 4 hrs. The anti-endotoxin effect of SEQ ID NO: 1 demonstrated in PBMC was statistically significant with p-value of <0.05 (**). (C) THP-1 cells were treated with LPS, SEQ ID NO: 1 or LPS+ SEQ ID NO: 1 for 4 hr in the absence (white bar) or presence of actinomycin D (black bar), the effect of actinomycin D on LPS-induced TNFα secretion was statistical significant with p-value<0.001 (***). (D) Cytoplasmic extracts of THP-1 cells treated with LPS, SEQ ID NO: 1 or LPS+ SEQ ID NO: 1 for 60 mins in the absence (black bar) or presence of monensin (white bar) were monitored by ELISA.

FIG. 7 is a graphical representation showing the anti-endotoxic effect of SEQ ID NO: 1 involves pre- and post-transcriptional events. Tissue culture supernatants were screened for TNFα by ELISA following stimulation of cells with 100 ng/ml of LPS in the absence (-▪-) or in the presence of 20 μg/ml SEQ ID NO: 1 (-●-) for 1, 2, 4 and 24 hr of treatment. In each case, the control indicates un-stimulated cells (-▾-), the y-axis represents TNFα concentration and the x-axis indicates time (hr). SEQ ID NO: 1 (20 ug/ml) was added (A) simultaneously with LPS, (B) after 30 min of LPS treatment, or (C) 30 min prior to LPS treatment. See materials and method for details. The results are an average (±standard deviation) of 3 independent experiments.

FIG. 8 is a graphical representation showing that SEQ ID NO: 1 modifies inflammatory agent-induced cytokine secretion by PBMC. PBMC were incubated alone or with TLR agonists (LPS, LTA, CpG) or inflammatory cytokines (TNFα, IL1β) for 4 or 24 hr in the presence (black bars) or absence (white bars) of SEQ ID NO: 1. See materials and methods in Example 13 for details. The concentration (y-axis) of IL1α, IL6, IL8 and TNFα(x-axis) were measured in the tissue culture supernatants by multiplex bead ELISA. The results are an average (±standard deviation) of 3 independent experiments. The effect of SEQ ID NO: 1 on agonist induced cytokine production was statistical significant with p-value<0.05 (***), p<0.1 (**) or p<0.15 (*).

FIG. 9 is a graphical representation showing an LPS-induced gene transcription profile in monocytes is altered by the presence of host defense peptide SEQ ID NO: 1. (A) THP-1 cells were stimulated with 100 ng/ml LPS in the absence (top panel) or presence (lower panel) of 20 ug/ml SEQ ID NO: 1 for 1, 2, 4 or 24 hr. Using microarray analysis, the gene expression in response to stimuli was calculated relative to that in unstimulated cells at each time point. The relative gene expression is overlaid on the TLR-4 protein network using the supervised clustering tool Cytoscape. The colour code for the fold change and identification of proteins are in the left panel. (B) Cluster analysis of the differentially expressed genes as measured using log ratio (y-axis) of microarray spot intensity, with NFκB binding sites in response to 100 ng/ml of LPS in the absence (top) or presence of 20 ug/ml of SEQ ID NO: 1 (bottom) based on similar temporal expression profiles over the time course of I to 24 hr (x-axis) using K-means, a no-hierarchical algorithm with an affinity threshold of 85%. The table indicates the total number of differentially expressed genes, total number of clusters, number of clusters containing genes with NFκB binding sites and the NFκB target genes found in the clusters.

FIG. 10 is a graphical representation showing that SEQ ID NO: 1 selectively modulates the transcription of LPS-induced pro-inflammatory genes. qPCR of gene expression in LPS-stimulated cells (-▪-), cells treated with SEQ ID NO: 1 alone (-▾-) or cells treated with a combination of LPS and SEQ ID NO: 1 (-●-) for 1,2,4, and 24 hr (x-axis). Results shown are an average (±standard error) of three independent experiments. Fold changes (y-axis, log scale) for each gene were normalized to GAPDH and are relative to the gene expression in un-stimulated cells (normalized to 1) using the comparative Ct method (see materials and methods in Example 13 for details).

FIG. 11 is a pictorial diagram and a graphical representation showing that SEQ ID NO: 1 suppresses LPS-induced translocation of NFκB subunits p50 and p65. (A) Western blot of NFκB subunits (identified on the right) in the nuclear extract of THP-1 cells following incubation in the absence (−) or presence (+) of 100 ng/ml LPS or LPS and 20 μg/ml SEQ ID NO: 1 for 60 mins. Pre-stained molecular mass markers are indicated on the left. (B) ELISA for NFκB subunit p50 (upper panel) and NFκB subunit p65 (lower panel) detected in the nuclear extracts of THP-1 cells stimulated for 60 min as described in (A). The y-axis represents relative light units (luminescence). See materials and methods in Example 13 for details. Results are representative of 3 independent experiments.

FIG. 12 is a pictorial diagram of a model describing mechanisms of anti-endotoxin activity of SEQ ID NO: 1. Based on the data presented herein, SEQ ID NO: 1 regulates LPS-induced gene transcription and cytokine production, by one or more of several mechanisms. (1) SEQ ID NO: 1 can interact directly with LPS to reduce its binding to LBP, MD2 or another component of the TLR4 receptor complex, thus reducing activation of the downstream pathway. (2) SEQ ID NO: 1 partially inhibits the TLR4→NFκB pathway and LPS-induced p50/p65 translocation probably by the action of certain negative regulators of NFκB (TNFAIP3, NFKBIA), the expression of which is relatively unaffected by SEQ ID NO: 1. (3) SEQ ID NO: 1 selectively modulates gene transcription; completely inhibiting certain pro-inflammatory genes (NFKB-1 (p50), TNFAIP2) and reducing the expression of others (TNFα). (4) SEQ ID NO: 1 directly triggers MAP kinase pathways that can impact on pro-inflammatory pathways. (5) SEQ ID NO: 1 has a stronger effect on e.g. TNFα protein production than on TNFα gene expression, and thus may directly or indirectly influence protein translation, stabilization, or processing. Points of intervention by SEQ ID NO: 1 are indicated by activation [symbol] inhibition (⊥), or suppression (→). Other abbreviations used are phosphorylation (P) and ubiquitination ( [symbol] ).

FIG. 13 is a pictorial diagram of a model describing mechanisms in which host defense peptides induce gene expression of the Janus Kinases and STAT family of transcription factors. Human PBMC were stimulated with (I) the human host defense peptide LL-37 (20 μg/ml) and (2) peptide SEQ ID NO: 7 for 4 hr. Using microarray analysis, the gene expression in CD14⁺ monocytes purified from the PBMC population in response to stimuli was calculated relative to that in un-stimulated cells. Differentially expressed genes were those with a fold change over the untreated control of 1.5-fold and a p-value<0.06 (calculated using a two-sided one-sample Student t-test on the log₂-ratios within each treatment group). The relative gene expression was overlaid onto a protein network using the systems biology clustering software tool Metacore™ (GeneGo, Inc., CA, USA). The color code for the fold changes are indicated as up-regulation (red) and down-regulation (blue) in response to the stimuli.

FIG. 14 is a graphical representation showing that SEQ ID NO: 7 induces transcription of genes functional in immune response. Quantitative real-time PCR of gene expression in human CDI4⁺ monocytes in response to host defense peptide SEQ ID NO: 7 after 4 hr of stimulation. Results shown are from four independent biological replicates (X-axis). Fold changes (Y-axis) for each gene were normalized to GAPDH and are relative to the gene expression in un-stimulated cells (normalized to 1) using the comparative Ct method. These represent markers of SEQ ID NO: 7 effects on blood cells.

FIG. 15 is a graphical representation showing that SEQ ID NO: 7 induces protein production in human PBMC within 4 hr of stimulation. PBMC were stimulated with SEQ ID NO: 7 (200 μg/ml) for 4 hours. The concentration (Y-axis) of cytokines IL-6 and IL-8 were measured in tissue culture supernatants by ELISA from PBMC of four individual donors (X-axis). The results shown are from four independent experiments.

FIG. 16 is a graphical representation showing that LPS-induced transcriptional responses in human monocytes are suppressed in the presence of SEQ ID NO: 7. Quantitative real-time PCR of gene expression in human CD14+ monocytes in response to LPS in the presence and absence of host defense peptide of SEQ ID NO: 7 after 4 hr of stimulation. Results shown are from four independent biological replicates (X-axis). Fold changes (Y-axis) for each gene were normalized to GAPDH, and are relative to the gene expression in un-stimulated cells (normalized to 1) using the comparative Ct method.

FIGS. 17A and 17B are graphical representations showing that SEQ ID NO: 7 suppresses LPS-induced pro-inflammatory TNF-o: secretion in human mononuclear cells within 4 hours of stimulation. Human PBMC and Human monocytic THP-1 cells were stimulated with LPS in the presence and absence of SEQ ID NO: 7 for 4 hours. The cells were treated with the peptide 45 mins prior to LPS stimulation. The concentration (Y-axis) of cytokines TNF-α was measured in tissue culture supernatants by ELISA. The results shown are from PBMC of three independent human donors. The results are an average (±standard deviation) of three independent experiments in THP-1 cells.

FIG. 18 is a graphical representation of a Venn diagram showing that the human host defense peptide LL-37 demonstrates both overlapping and distinct induction of differentially expressed (DE) and statistically significant genes compared to SEQ ID NO: 7.

FIG. 19 is a pictorial diagram showing that protein levels of total IιBα diminish within 30 min and return to control levels by 60 min in THP-1 cells, indicating that LL-37 may directly modulate elements of the LPS signaling pathway.

FIGS. 20A and 20B are graphical representations showing data from fresh isolated human PBMCs that were incubated with IL-1β (10 ng/ml) or LPS (100 ng/ml) in absence or presence of LL-37 (20 ug/ml) for 24 hours. IL-6 and MCP-3 ELISA were performed to measure the level of protein release.

FIGS. 21A and 21B are pictorial diagrams showing Western blots for cytoplasm protein and nuclear protein. The combined treatment of LL-37 and IL-1 β showed higher IκBα phosphorylation after 30 min and p50 nuclear translocation after 60 min than LL-37 or IL-1β treatment alone in human PBMCs. Similar translocation results were also observed in THP-1 cells at an earlier time point (20 min after treatment). In addition, LL-37 alone induced NFκB subunit p50 translocation in both human PBMCs and THP-1 cells.

FIGS. 22A-22D are graphical representations showing data from human PBMCs that were pre-treated with PI3 kinase inhibitor, LY294002 (25 μM) for 1 h, followed by incubation with IL-1β (10 ng/ml) in the presence or absence of LL-37 (20 μg/ml) for 24 hours. The LL-37 plus IL-1β mediated production of IL-6 and MCP-3 was significantly inhibited by LY294002 pre-incubation, indicating that P13 kinase plays a role in LL-37-induced modulation of cytokine and chemokine production.

FIG. 23 is a pictorial diagram showing that activation of PI3 kinase causes activation of a number of intracellular signal transduction pathways, including phosphorylation of the downstream target protein kinase B (Akt). Further analysis showed that phosphorylation of CREB was observed in human PBMCs after exposure to LL-37 for 30min and 60min. Activation of Akt and CREB was augmented by the presence of IL-1β (10 ng/ml).

### DETAILED DESCRIPTION OF THE INVENTION

The present invention provides novel cationic peptides, characterized by a group of generic formulas (SEQ ID NO: 4, 11, 18, 25, 32, 39, 46), which have ability to modulate (e.g., up- and/or down regulate) polynucleotide expression, thereby regulating sepsis and inflammatory responses and/or innate immunity.

“Innate immunity” as used herein refers to the natural ability of an organism to defend itself against invasions by pathogens. Pathogens or microbes as used herein, may include, but are not limited to bacteria, fungi, parasite, and viruses. Innate immunity is contrasted with acquired/adaptive immunity in which the organism develops a defensive mechanism based substantially on antibodies and/or immune lymphocytes that is characterized by specificity, amplifiability and self vs. non-self dsicrimination. With innate immunity, broad, nonspecific immunity is provided and there is no immunologic memory of prior exposure. The hallmarks of innate immunity are effectiveness against a broad variety of potential pathogens, independence of prior exposure to a pathogen, and immediate effectiveness (in contrast to the specific immune response which takes days to weeks to be elicited). In addition, innate immunity includes immune responses that affect other diseases, such as cancer, inflammatory diseases, multiple sclerosis, various viral infections, and the like.

As used herein, the term “cationic peptide” refers to a sequence of amino acids from about 5 to about 50 amino acids in length. In one aspect, the cationic peptide of the invention is from about 10 to about 35 amino acids in length. A peptide is “cationic” if it possesses sufficient positively charged amino acids to have a pI greater than about 9.0, where pl (isoelectric point)=pH when the net charge of the peptide is neutral. Typically, at least two of the amino acid residues of the cationic peptide will be positively charged, for example, lysine or arginine. “Positively charged” refers to the side chains of the amino acid residues which have a net positive charge at pH 7.0. Examples of naturally occurring cationic antimicrobial peptides which can be recombinantly produced according to the invention include defensins, cathelicidins, magainins, melittin, and cecropins, bactenecins, indolicidins, polyphemusins, tachyplesins, and analogs thereof. A variety of organisms make cationic peptides, molecules used as part of a non-specific defense mechanism against microorganisms. When isolated, these peptides are toxic to a wide variety of microorganisms, including bacteria, fuingi, and certain enveloped viruses. While cationic peptides act against many.pathogens, notable exceptions and varying degrees of toxicity exist. However this patent reveals additional cationic peptides with no toxicity towards microorganisms but an ability to protect against infections through stimulation of innate immunity, and this invention is not limited to cationic peptides with antimicrobial activity. In fact, many peptides useful in the present invention do not have antimicrobial activity.

Cationic peptides known in the art include for example, the human cathelicidin LL-37, and the bovine neutrophil peptide indolicidin and the bovine variant of bactenecin, Bac2A.

(SEQ ID NO: 1)

| | |
|---|---|
| LL-37 | LLGDFFRKSKEKIGKEFKRIVQRIKDFLRNLVPRTES |

(SEQ ID NO: 2)

| | |
|---|---|
| Indolicidin | ILPWKWPWWPWRR-NH₂ |

(SEQ ID NO: 3)

| | |
|---|---|
| Bac2A | RLARIVVIRVAR-NH₂ |

Although SEQ ID NO: 1 is often defined as an antimicrobial (direct killing) peptide it has been suggested that at physiological salt conditions, this peptide is not antimicrobial at the concentrations (1-5 μg/ml) normally found in adults at mucosal surfaces (Bowdish, D. M. E., D. J. Davidson, Y. E. Lau, K. Lee, M. G. Scott, and R. E. W. Hancock. 2005. Impact of LL-37 on anti-infective immunity. J. Leukocyte Biol. 77:451-459). Moreover under these conditions and at these concentrations, SEQ ID NO: 1 exhibits a variety of immunomodulatory functions. This could help to explain why SEQ ID NO: 1 administration can protect mice against certain bacterial infections, due to its ability to modulate immunity. SEQ ID NO: 1 is also able to protect mice and rats against endotoxemialsepsis induced by pure LPS indicating that SEQ ID NO: 1 can suppress potentially harmful pro-inflammatory responses.

Accordingly, the present invention provides evidence that human host defense peptide SEQ ID NO: 1 has potent anti-endotoxin properties, at very low (≦1 μg/ml) concentrations and physiological salt conditions reflecting those found in vivo. It is further demonstrated here that SEQ ID NO: 1 had a general anti-inflammatory effect on TLR stimulation, inhibiting pro-inflammatory cytokine release from human monocytic cells stimulated with TLR2, TLR4 and TLR9 agonists. The suppression of inflammatory responses by SEQ ID NO: 1 in LPS-stimulated cells is selective, as SEQ ID NO: 1 does not block the expression of certain (pro-inflammatory) genes required for cell recruitment and movement, yet abrogates pro-inflammatory cytokine responses that can potentially lead to sepsis. The anti-inflammatory activity of SEQ ID NO: 1 is apparently mediated through a diversity of mechanisms.

In innate immunity, the immune response is not dependent upon antigens. The innate immunity process may include the production of secretory molecules and cellular components as set forth above. In innate immunity, the pathogens are recognized by receptors (for example, Toll-like receptors) that have broad specificity, are capable of recognizing many pathogens, and are encoded in the germline. These Toll-like receptors have broad specificity and are capable of recognizing many pathogens. When cationic peptides are present in the immune response, they aid in the host response to pathogens. This change in the immune response induces the release of chemokines, which promote the recruitment of immune cells to the site of infection.

Chemokines, or chemoattractant cytokines, are a subgroup of immune factors that mediate chemotactic and other pro-inflammatory phenomena (See, Schall, 1991, Cytokine 3:165-183). Chemokines are small molecules of approximately 70-80 residues in length and can generally be divided into two subgroups, α which have two N-terminal cysteines separated by a single amino acid (CxC) and β which have two adjacent cysteines at the N terminus (CC). RANTES, MIP-1α and MIP-1β are members of the β subgroup (reviewed by Horuk, R., 1994, Trends Pharmacol. Sci, 15:159-165; Murphy, P. M., 1994, Annu. Rev. Immunol., 12:593-633). The amino terminus of the β chemokines RANTES, MCP-1, and MCP-3 have been implicated in the mediation of cell migration and inflammation induced by these chemokines. This involvement is suggested by the observation that the deletion of the amino terminal 8 residues of MCP-1, amino terminal 9 residues of MCP-3, and amino terminal 8 residues of RANTES and the addition of a methionine to the amino terminus of RANTES, antagonize the chemotaxis, calcium mobilization and/or enzyme release stimulated by their native counterparts (Gong et al., 1996 J. Biol. Chem. 271:10521-10527; Proudfoot et al., 1996 J Biol. Chem. 271:2599-2603). Additionally, α chemokine-like chemotactic activity has been introduced into MCP-1 via a double mutation of Tyr 28 and Arg 30 to leucine and valine, respectively, indicating that internal regions of this protein also play a role in regulating chemotactic activity (Beall et al., 1992, J. Biol. Chem. 267:3455-3459).

The monomeric forms of all chemokines characterized thus far share significant structural homology, although the quaternary structures of α and β groups are distinct. While the monomeric structures of the β and a chemokines are very similar, the dimeric structures of the two groups are completely different. An additional chemokine, lymphotactin, which has only one N terminal cysteine has also been identified and may represent an additional subgroup (γ) of chemokines (Yoshida et al., 1995, FEBS Lett. 360:155-159; and Kelner et al., 1994, Science 266:1395-1399).

Receptors for chemokines belong to the large family of G-protein coupled, 7 transmembrane domain receptors (GCR's) (See, reviews by Horuk, R., 1994, Trends Pharmacol. Sci. 15:159-165; and Murphy, P. M., 1994, Annu. Rev. Immunol. 12:593-633). Competition binding and cross-desensitization studies have shown that chemokine receptors exhibit considerable promiscuity in ligand binding. Examples demonstrating the promiscuity among β chemokine receptors include: CC CKR-1, which binds RANTES and MIP-1α (Neote et al., 1993, Cell 72: 415-425), CC CKR-4, which binds RANTES, MIP-1α, and MCP-1 (Power et al., 1995, J. BioL. Chem. 270:19495-19500), and CC CKR-5, which binds RANTES, MIP-1α, and MIP-1β (Alkhatib et al., 1996, Science, in press and Dragic et al., 1996, Nature 381:667-674). Erythrocytes possess a receptor (known as the Duffy antigen) which binds both α and β chemokines (Horuk et al., 1994, J. Biol. Chem. 269:17730-17733; Neote et al., 1994, Blood 84:44-52; and Neote et al., 1993, J. Biol. Chem. 268:12247-12249). Thus the sequence and structural homologies evident among chemokines and their receptors allows some overlap in receptor-ligand interactions.

In one aspect, the present invention provides the use of compounds including peptides of the invention to reduce sepsis and inflammatory responses by acting directly on host cells. In this aspect, a method of identification of a polynucleotide or polynucleotides that are regulated by one or more sepsis or inflammatory inducing agents is provided, where the regulation is altered by a cationic peptide. Such sepsis or inflammatory inducing agents include, but are not limited to endotoxic lipopolysaccharide (LPS), lipoteichoic acid (LTA) and/or CpG DNA or intact bacteria or other bacterial components. The identification is performed by contacting the polynucleotide or polynucleotides with the sepsis or inflammatory inducing agents and further contacting with a cationic peptide either simultaneously or immediately after. The expression of the polynucleotide in the presence and absence of the cationic peptide is observed and a change in expression is indicative of a polynucleotide or pattern of polynucleotides that is regulated by a sepsis or inflammatory inducing agent and inhibited by a cationic peptide. In another aspect, the invention provides a polynucleotide identified by the method.

Once identified, such polynucleotides will be useful in methods of screening for compounds that can block sepsis or inflammation by affecting the expression of the polynucleotide. Such an effect on expression may be either up regulation or down regulation of expression. By identifying compounds that do not trigger the sepsis reaction and that can block or dampen inflammatory or septic responses, the present invention also presents a method of identifying enhancers of innate immunity. Additionally, the present invention provides compounds that are used or identified in the above methods.

Candidate compounds are obtained from a wide variety of sources including libraries of synthetic. or natural compounds. For example, numerous means are available for, random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, and the like to produce structural analogs. Candidate agents are also found among biomolecules including, but not limited to: peptides, peptidiomimetics, saccharides, fatty acids, steroids, purines, pyrimidines, polypeptides, polynucleotides, chemical compounds, derivatives, structural analogs or combinations thereof.

Incubating components of a screening assay includes conditions which allow contact between the test compound and the polynucleotides of interest. Contacting includes in solution and in solid phase, in a cell, or on a cell surface. The test compound may optionally be a combinatorial library for screening a plurality of compounds. Compounds identified in the method of the invention can be further evaluated, detected, cloned, sequenced, and the like, either in solution or after binding to a solid support, by any method usually applied to the detection of a compound.

Generally, in the methods of the invention, a cationic peptide is utilized to detect and locate a polynucleotide that is essential in the process of sepsis or inflammation. Once identified, a pattern of polynucleotide expression may be obtained by observing the expression in the presence and absence of the cationic peptide. The pattern obtained in the presence of the cationic peptide is then useful in identifying additional compounds that can inhibit expression of the polynucleotide and therefore block sepsis or inflammation. It is well known to one of skill in the art that non-peptidic chemicals and peptidomimetics can mimic the ability of peptides to bind to receptors and enzyme binding sites and thus can be used to block or stimulate biological reactions. Where an additional compound of interest provides a pattern of polynucleotide expression similar to that of the expression in the presence of a cationic peptide, that compound is also useful in the modulation of sepsis or an innate immune response. In this manner, the cationic peptides of the invention, which are known inhibitors of sepsis and inflammation and enhancers of innate immunity are useful as tools in the identification of additional compounds that inhibit sepsis and inflammation and enhance innate immunity.

As can be seen in the Examples below, peptides of the invention have a widespread ability to reduce the expression of polynucleotides regulated by LPS. High levels of endotoxin in the blood-are responsible for many of the symptoms seen during a serious infection or inflammation such as fever and an elevated white blood cell count. Endotoxin is a component of the cell wall of Gram-negative bacteria and is a potent trigger of the pathophysiology of sepsis. The basic mechanisms of inflammation and sepsis are related. In Example 1, polynucleotide arrays were utilized to determine the effect of cationic peptides on the transcriptional response of epithelial cells. Specifically, the effects on over 14,000 different specific polynucleotide probes induced by LPS were observed. The tables show the changes seen with cells treated with peptide compared to control cells. The resulting data indicated that the peptides have the ability to reduce the expression of polynucleotides induced by LPS.

Example 2, similarly, shows that peptides of the invention are capable of neutralizing the stimulation of immune cells by Gram positive and Gram negative bacterial products. Additionally, it is noted that certain pro-inflammatory polynucleotides are down-regulated by cationic peptides, as set forth in table 24 such as TLR1 (AI339155), TLR2 (T57791), TLR5 (N41021), TNF receptor-associated factor 2 (T55353), TNF receptor-associated factor 3 (AA504259), TNF receptor superfamily, member 12 (W71984), TNF receptor superfamily, member 17 (AA987627), small inducible cytokine subfamily B, member 6 (AI889554), IL-12R beta 2 (AA977194), IL-18 receptor 1 (AA482489), while anti-inflammatory polynucleotides are up-regulated by cationic peptides, as seen in table 25 such as IL-1 R antagonist homolog 1 (AI167887), IL-10 R beta (AA486393), TNF Receptor member 1B (AA150416), TNF receptor member 5 (H98636), TNF receptor member 11b (AA194983), IK cytokine down-regulator of HLA II (R39227), TGF-B inducible early growth response 2 (AI473938), or CD2 (AA927710). The relevance and application of these results are confirmed by an in vivo application to mice.

In another aspect, the invention provides a method of identifying an agent that enhances innate immunity. In-the method, a polynucleotide or polynucleotides that encode a polypeptide involved in innate immunity is contacted with an agent of interest. Expression of the polynucleotide is determined, both in the presence and absence of the agent. The expression is compared and of the specific modulation of expression was indicative of an enhancement of innate immunity. In another aspect, the agent does not stimulate a septic reaction as revealed by the lack of upregulation of the pro-inflammatory cytokine TNF-α. In still another aspect the agent reduces or blocks the inflammatory or septic response. In yet another aspect, the agent reduces the expression of TNF-αand/or interleukins including, but not limited to, IL-1β, IL-6, IL-12 p40, IL-12 p70, and IL-8.

In another aspect, the invention provides methods of direct polynucleotide regulation by cationic peptides and the use of compounds including cationic peptides to stimulate elements of innate immunity. In this aspect, the invention provides a method of identification of a pattern of polynucleotide expression for identification of a compound that enhances innate immunity. In the method of the invention, an initial detection of a pattern of polynucleotide expression for cells contacted in the presence and absence of a cationic peptide is made. The pattern resulting from polynucleotide expression in the presence of the peptide represents stimulation of innate immunity. A pattern of polynucleotide expression is then detected in the presence of a test compound, where a resulting pattern with the test compound that is similar to the pattern observed in the presence of the cationic peptide is indicative of a compound that enhances innate immunity. In another aspect, the invention provides compounds that are identified in the above methods. In another aspect, the compound of the invention stimulates chemokine or chemokine receptor expression. Chemokine or chemokine receptors may include, but are not limited to CXCR4, CXCR1, CXCR2, CCR2, CCR4, CCR5, CCR6, MIP-1 alpha, MDC, MIP-3 alpha, MCP-1, MCP-2, MCP-3, MCP4, MCP-5, anid RANTES. In still another aspect, the compound is a peptide, peptidomimetic, chemical compound, or a nucleic acid molecule.

In still another aspect the polynucleotide expression pattern includes expression of pro-inflammatory polynucleotides. Such pro-inflammatory polynucleotides may include, but are not limited to, ring finger protein 10 (D87451), serine/threonine protein kinase MASK (AB040057), KIAA0912 protein (AB020719), KIAA0239 protein (D87076), RAP1, GTPase activating protein 1 (M64788), FEM-1-like death receptor binding protein (AB007856), cathepsin S (M90696), hypothetical protein FLJ20308 (AK000315), pim-1 oncogene (M54915), proteasome subunit beta type 5 (D2901 1), KIAA0239 protein (D87076), mucin 5 subtype B tracheobronchial (AJ001403), cAMP response element-binding protein CREBPa, integrin alpha M (J03925), Rho-associated kinase 2 (NM_{—}004850), PTD017 protein (AL050361) unknown genes (AK00143, AK034348, AL049250, AL161991, AL031983) and any combination thereof In still another aspect the polynucleotide expression pattern includes expression of cell surface receptors that may include but is not limited to retinoic acid receptor (X06614), G protein-coupled receptors (Z94155, X81892, U52219, U22491, AF015257, U66579) chemokine (C-C motif) receptor 7 (L31584), tumor necrosis factor receptor superfamily member 17 (Z29575), interferon gamma receptor 2 (U05875), cytokine receptor-like factor 1 (AF059293), class I cytokine receptor (AF053004), coagulation factor II (thrombin) receptor-like 2 (U9297 1), leukemia inhibitory factor receptor (NM_{—}002310), interferon gamma receptor 1 (AL050337).

In Example 4 it can be seen that the cationic peptides of the invention alter polynucleotide expression in macrophage and epithelial cells. The results of this example-show that pro-inflammatory polynucleotides are down-regulated by cationic peptides (Table 24) whereas anti-inflammatory polynucleotides are up-regulated by cationic peptides (Table 25).

It is shown below, for example, in tables 1-15, that cationic peptides can neutralize the host response to the signaling molecules of infectious agents as well as modify the transcriptional responses of host cells, mainly by down-regulating the pro-inflammatory response and/or up-regulating the anti-inflammatory response. Example 5 shows that the cationic peptides can aid in the host response to pathogens by inducing the release of chemokines, which promote the recruitment of immune cells to the site of infection. The results are confirmed by an in vivo application to mice.

It is seen from the examples below that cationic peptides have a substantial influence on the host response to pathogens in that they assist in regulation of the host immune response by inducing selective pro-inflammatory responses that for example promote the recruitment of immune cells to the site of infection but not inducing potentially harmful pro-inflammatory cytokines. Sepsis appears to be caused in part by an overwhelming pro-inflammatory response to infectious agents. Peptides can aid the host in a “balanced” response to pathogens by inducing an anti-inflammatory response and suppressing certain potentially harmful pro-inflammatory responses.

In Example 7, the activation of selected MAP kinases was examined, to study the basic mechanisms behind the effects of interaction of cationic peptides with cells. Macrophages activate MEK/ERK kinases in response to bacterial infection. MEK is a MAP kinase kinase that when activated, phosphorylates the downstream kinase ERK (extracellular regulated kinase), which then dimerizes and translocates to the nucleus where it activates transcription factors such as Elk-1 to modify polynucleotide expression. MEK/ERK kinases have been shown to impair replication of Salmonella within macrophages. Signal transduction by MEK kinase and NADPH oxidase may play an important role in innate host defense against intracellular pathogens. By affecting the MAP kinases as shown below the cationic peptides have an effect on bacterial infection. The cationic peptides can directly affect kinases. Table 21 demonstrates but is not limited to MAP kinase polynucleotide expression changes in response to peptide. The kinases include MAP kinase kinase 6 (H070920), MAP kinase kinase 5 (W69649), MAP kinase 7 (H39192), MAP kinase 12 (AI936909) and MAP kinase-activated protein kinase 3 (W6828 1).

In another method, the methods of the invention may be used in combination, to identify an agent with multiple characteristics, i.e. a peptide with anti-inflammatory/anti-sepsis activity, and the ability to enhance innate immunity, in part by inducing chemokines in vivo.

In another aspect, the invention provides a method for inferring a state of infection in a mammalian subject from a nucleic acid sample of the subject by identifying in the nucleic acid sample a polynucleotide expression pattern exemplified by an increase in polynucleotide expression of at least 2 polynucleotides in Table 55 as compared to a non-infected subject. In another aspect the invention provides a method for inferring a state of infection in a mammalian subject from a nucleic acid sample of the subject by identifying in the nucleic acid sample a polynucleotide expression pattern exemplified by a polynucleotide expression of at least 2 polynucleotides in Table 56 or Table 57 as compared to a non-infected subject. In one aspect of the invention, the state of infection is due to infectious agents or signaling molecules derived therefrom, such as, but not limited to, Gram negative bacteria and Gram positive bacteria, viral, fungal or parasitic agents. In still another aspect the invention provides a polynucleotide expression pattern of a subject having a state of infection identified by the above method. Once identified, such polynucleotides will be useful in methods of diagnosis of a condition associated with the activity or presence of such infectious agents or signaling molecules.

Example 10 below demonstrates this aspect of the invention. Specifically, table 61 demonstrates that both MEK and the NADPH oxidase inhibitors can limit bacterial replication (infection of IFN-γ-primed macrophages by S. typhimurium triggers a MEK kinase). This is an example of how bacterial survival can be impacted by changing host cell signaling molecules.

In still another aspect of the invention, compounds are presented that inhibit stromal derived factor-1 (SDF-1) induced chemotaxis of T cells. Compounds are also presented which decrease expression of SDF-1 receptor. Such compounds also may act as an antagonist or inhibitor of CXCR-4. In one aspect the invention provides a cationic peptide that is an antagonist of CXCR-4. In another aspect the invention provides a method of identifying a cationic peptide that is an antagonist of CXCR-4. The method includes contacting T cells with SDF-1 in the presence of absence of a test peptide and measuring chemotaxis. A decrease in chemotaxis in the presence of the test peptide is then indicative of a peptide that is an antagonist of CXCR-4. Such compounds and methods are useful in therapeutic applications in HIV patients. These types of compounds and the utility thereof is demonstrated, for example, in Example 11 (see also Tables 62, 63). In that example, cationic peptides are shown to inhibit cell migration and therefore antiviral activity.

In one embodiment, the invention provides an isolated cationic peptides having an amino acid sequence of the general formula (Formula A): X₁X₂X₃IX₄PX₄IPX₅X₂X₁ (SEQ ID NO: 4), wherein X₁ is one or two of R, L or K, X₂ is one of C, S or A, X₃ is one of R or P, X₄ is one of A or V and X₅ is one of V or W. Examples of-the peptides of the invention include, but are not limited to: LLCRIVPVIPWCK (SEQ ID NO: 5), LRCPIAPVIPVCKK (SEQ ID NO: 6), KSRIVPAIPVSLL (SEQ ID NO: 7), KKSPIAPAIPWSR (SEQ ID NO: 8), RRARIVPAIPVARR (SEQ ID NO: 9) and LSRIAPAIPWAKL (SEQ ID NO: 10).

In another embodiment, the invention provides an isolated linear cationic peptide having an amino acid sequence of the general formula (Formula B):

X₁LX₂X₃KX₄X₂X₅X₃PX₃X₁ (SEQ ID NO: 11), wherein XI is one or two of D, E, S, T or N, X2 is one or two of P, G or D, X₃ is one of G, A, V, L, I or Y, X₄ is one of R, K or H and X₅ is one of S, T, C, M or R. Examples of the peptides of the invention include, but are not limited to:

| | |
|---|---|
| DLPAKRGSAPGST, | (SEQ ID NO: 12) |
| SELPGLKHPCVPGS, | (SEQ ID NO: 13) |
| TTLGPVKRDSIPGE, | (SEQ ID NO: 14) |
| SLPIKHDRLPATS, | (SEQ ID NO: 15) |
| ELPLKRGRVPVE | (SEQ ID NO: 16) |
| and | |
| NLPDLKKPRVPATS. | (SEQ ID NO: 17) |

In another embodiment, the invention provides an isolated linear cationic peptide having an amino acid sequence of the general formula (Formula C): X₁X₂X₃X₄WX₄WX₄X₅K (SEQ ID NO: 18) (this formula includes CP12a and CP12d), wherein X₁ is one to four chosen from A, P or R, X₂ is one or two aromatic amino acids (F, Y and W), X₃ is one of P or K, X₄ is one, two or none chosen from A, P, Y or W and X₅ is one to three chosen from R or P. Examples of the peptides of the invention include, but are not limited to:

| | |
|---|---|
| RPRYPWWPWWPYRPRK, | (SEQ ID NO: 19) |
| RRAWWKAWWARRK, | (SEQ ID NO: 20) |
| RAPYWPWAWARPRK, | (SEQ ID NO: 21) |
| RPAWKYWWPWPWPRRK, | (SEQ ID NO: 22) |
| RAAFKWAWAWWRRK | (SEQ ID NO: 23) |
| and | |
| RRRWKWAWPRRK. | (SEQ ID NO: 24) |

In another embodiment, the invention provides an isolated hexadecameric cationic peptide having an amino acid sequence of the general formula (Formula D):

X₁X₂X₃X₄X₁VX₃X₄RGX₄X₃X₄X₁X₃X₁ (SEQ ID NO: 25) wherein X₁ is one or two of R or K, X₂ is a polar or charged amino acid (S, T, M, N, Q, D, E, K, R and H), X₃ is C, S, M, D or A and X4 is F, I, V, M or R. Examples of the peptides of the invention include, but are not limited to: RRMCIKVCVRGVCRRKCRK (SEQ ID NO: 26), KRSCFKVSMRGVSRRRCK (SEQ ID NO: 27), KKDAIKKVDIRGMDMRRAR (SEQ ID NO: 28), RKMVKVDVRGIMIRKDRR (SEQ ID NO: 29), KQCVKVAMRGMALRRCK (SEQ ID NO: 30) and RREAIRRVAMRGRDMKRMRR (SEQ ID NO: 31).

In still another embodiment, the invention provides an isolated hexadecameric cationic peptide having an amino acid sequence of the general formula (Formula E): X₁X₂X₃X₄X₁VX₅X₄RGX₄X₅X₄X₁X₃X₁ (SEQ ID NO: 32), wherein XI is one or two of R or K, X₂ is a polar or charged amino acid (S, T, M, N, Q, D, E, K, R and H), X₃ is one of C, S, M, D or A, X₄ is one of F, I, V, M or R and X₅ is one of A, I, S, M, D or R. Examples of the peptides of the invention include, but are not limited to: RTCVKRVAMRGIIRKRCR (SEQ ID NO: 33), KKQMMKRVDVRGISVKRKR (SEQ ID NO: 34), KESIKVIIRGMMVRMKK (SEQ ID NO: 35), RRDCRRVMVRGIDIKAK (SEQ ID NO: 36), KRTAIKKVSRRGMSVKARR (SEQ ID NO: 37) and RHCIRRVSMRGIIMRRCK (SEQ ID NO: 38).

In another embodiment, the invention provides an isolated longer cationic peptide having an amino acid sequence of the general formula (Formula F): KX₁KX₂FX₂KMLMX₂ALKKX₃ (SEQ ID NO: 39), wherein X₁ is a polar amino acid (C, S, T, M, N and Q); X₂ is one of A, L, S or K and X₃ is 1-17 amino acids chosen from G, A, V, L, I, P, F, S, T, K and H. Examples of the peptides of the invention include, but are not limited to:

| | |
|---|---|
| KCKLFKKMLMLALKKVLTTGLPALKLTK, | (SEQ ID NO: 40) |
| KSKSFLKMLMKALKKVLTTGLPALIS, | (SEQ ID NO: 41) |
| KTKKFAKMLMMALKKVVSTAKPLAILS, | (SEQ ID NO: 42) |
| KMKSFAKMLMLALKKVLKVLTTALTLKAGLPS, | (SEQ ID NO: 43) |
| KNKAFAKMLMKALKKVTTAAKPLTG | (SEQ ID NO: 44) |
| and | |
| KQKLFAKMLMSALKKKTLVTTPLAGK. | (SEQ ID NO: 45) |

In yet another embodiment, the invention provides an isolated longer cationic peptide having an amino acid sequence of the general formula (Formula G): KWKX₂X₁X₁X₂X₂X₁X₂X₂X_{X}₁X₂X₂IFHTALKPISS (SEQ ID NO: 46), wherein X₁ is a hydrophobic amino acid and X₂ is a hydrophilic amino acid. Examples of the peptides of the invention include, but are not limited to:

| | |
|---|---|
| KWKSFLRTKFSPVRTIFHTALKPISS, | (SEQ ID NO: 47) |
| KWKSYAHTIMSPVRLIFHTALKPISS, | (SEQ ID NO: 48) |
| KWKRGAHRFMKFLSTIFHTALKPISS, | (SEQ ID NO: 49) |
| KWKKWAHSPRKVLTRIFHTALKPISS, | (SEQ ID NO: 50) |
| KWKSLVMMFKKPARRIFHTALKPISS | (SEQ ID NO: 51) |
| and | |
| KWKHALMKAHMLWHMIFHTALKPISS. | (SEQ ID NO: 52) |

In still another embodiment, the invention provides an isolated cationic peptide having an amino acid sequence of the formula: KWKSFLRTFKSPVRTVFHTALKPISS (SEQ ID NO: 53) or KWKSYAHTIMSPVRLVFHTALKPISS (SEQ ID NO: 54).

The termn “isolated” as used herein refers to a peptide that is substantially free of other proteins, lipids, and nucleic acids (e.g., cellular components with which an in vivo-produced peptide would naturally be associated). Preferably, the peptide is at least 70%, 80%, or most preferably 90% pure by weight and when assessed in exclusion of counter-ion.

The invention also includes analogs, derivatives, conservative variations, and cationic peptide variants of the enumerated polypeptides, provided that the analog, derivative, conservative variation, or variant has a detectable activity in which it enhances innate immunity or has anti-inflammatory activity. It is not necessary that the analog, derivative, variation, or variant have activity identical to the activity of the peptide from which the analog, derivative, conservative variation, or variant is derived.

A cationic peptide “variant” is a peptide that is an altered form of a referenced cationic peptide. For example, the term “variant” includes a cationic peptide in which at least one amino acid of a reference peptide is substituted in an expression library. The term “reference” peptide means any of the cationic peptides of the invention (e.g. as defined in the above formulas), from which a variant, derivative, analog, or conservative variation is derived. Included within the term “derivative” is a hybrid peptide that includes at least a portion of each of two cationic peptides (e.g., 30-80% of each of two cationic peptides). Also included are peptides in which one or more amino acids are deleted from the sequence of a peptide enumerated herein, provided that the derivative has activity in which it enhances innate immunity or has anti-inflammatory activity. This can lead to the development of a smaller active molecule which would also have utility. For example, amino or carboxy terminal amino acids which may not be required for enhancing innate immunity or anti-inflammatory activity of a peptide can be removed. Likewise, additional derivatives can be produced by adding one or a few (e.g., less than 5) amino acids to a cationic peptide without completely inhibiting the activity of the peptide. In addition, C-terminal derivatives, e.g., C-terminal methyl esters, and N-terminal derivatives can be produced and are encompassed by the invention. Peptides of the invention include any analog, homolog, mutant, isomer or derivative of the peptides disclosed in the present invention, so long as the bioactivity as described herein remains. Also included is the reverse sequence of a peptide encompassed by the general formulas set forth above. Additionally, an amino acid of “D” configuration may be substituted with an amino acid of “L” configuration and vice versa. Alternatively the peptide may be cyclized chemically or by the addition of two or more cysteine residues within the sequence and oxidized to form disulphide bonds.

The invention also includes peptides that are conservative variations of those peptides exemplified herein. The term “conservative variation” as used herein denotes a polypeptide in which at least one amino acid is replaced by another, biologically similar residue. Examples of conservative variations include the substitution of one hydrophobic residue, such as isoleucine, valine, leucine, alanine, cysteine, glycine, phenylalanine, proline, tryptophan, tyrosine, norleucine or methionine for another, or the substitution of one polar residue for another, such as the substitution of arginine for lysine, glutamic for aspartic acid, or glutamine for asparagine, and the like. Neutral hydrophilic amino acids that can be substituted for one another include asparagine, glutamine, serine and threonine. The term “conservative variation” also encompasses a peptide having a substituted amino acid in place of an unsubstituted parent amino acid. Such substituted amino acids may include amino acids that have been methylated or amidated. Other substitutions will be known to those of skill in the art. In one aspect, antibodies raised to a substituted polypeptide will also specifically bind the unsubstituted polypeptide.

Peptides of the invention can be synthesized by commonly used methods such as those that include t-BOC or FMOC protection of alpha-amino groups. Both methods involve stepwise synthesis in which a single amino acid is added at each step starting from the C-terminus of the peptide (See, Coligan, et al., Current Protocols in Immunology, Wiley Interscience, 1991, Unit 9). Peptides of the invention can also be synthesized by the well known solid phase peptide synthesis methods such as those described by Merrifield, J. Am. Chem. Soc., 85:2149, 1962) and Stewart and Young, Solid Phase Peptides Synthesis, Freeman, San Francisco, 1969, pp. 27-62) using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with a 1% acetic acid solution, which is then lyophilized to yield the crude material. The peptides can be purified by such techniques as gel filtration on Sephadex G-15 using 5% acetic acid as a solvent. Lyophilization of appropriate fractions of the column eluate yield homogeneous peptide, which can then be characterized by standard techniques such as amino acid analysis, thin layer chromatography, high performance liquid chromatography, ultraviolet absorption spectroscopy, molar rotation, or measuring solubility. If desired, the peptides can be quantitated by the solid phase Edman degradation.

The invention also includes isolated nucleic acids (e.g., DNA, cDNA, or RNA) encoding the peptides of the invention. Included are nucleic acids that encode analogs, mutants, conservative variations, and variants of the peptides described herein. The term “isolated” as used herein refers to a nucleic acid that is substantially free of proteins, lipids, and other nucleic acids with which an in vivo-produced nucleic acids naturally associated. Preferably, the nucleic acid is at least 70%, 80%, or preferably 90% pure by weight, and conventional methods for synthesizing nucleic acids in vitro can be used in lieu of in vivo methods. As used herein, “nucleic acid” refers to a polymer of deoxyribo-nucleotides or ribonucleotides, in the form of a separate fragment or as a component of a larger genetic construct (e.g., by operably linking a promoter to a nucleic acid encoding a peptide of the invention). Numerous genetic constructs (e.g., plasmids and other expression vectors) are known in the art and can be used to produce the peptides of the invention in cell-free systems or prokaryotic or eukaryotic (e.g., yeast, insect, or mammalian) cells. By taking into account the degeneracy of the genetic code, one of ordinary skill in the art can readily synthesize nucleic acids encoding the polypeptides of the invention. The nucleic acids of the invention can readily be used in conventional molecular biology methods to produce the peptides of the invention.

DNA encoding the cationic peptides of the invention can be inserted into an “expression vector.” The term “expression vector” refers to a genetic construct such as a plasmid, virus or other vehicle known in the art that can be engineered to contain a nucleic acid encoding a polypeptide of the invention. Such expression vectors are preferably plasmids that contain a promoter sequence that facilitates transcription of the inserted genetic sequence in a host cell. The expression vector typically contains an origin of replication, and a promoter, as well as polynucleotides that allow phenotypic selection of the transformed cells (e.g., an antibiotic resistance polynucleotide). Various promoters, including inducible and constitutive promoters, can be utilized in the invention. Typically, the expression vector contains a replicon site and control sequences that are derived from a species compatible with the host cell.

Transformation or transfection of a recipient with a nucleic acid of the invention can be carried out using conventional techniques well known to those skilled in the art. For example, where the host cell is E. coli, competent cells that are capable of DNA uptake can be prepared using the CaCI₂, MgCl₂ or RbCl methods known in the art. Alternatively, physical means, such as electroporation or microinjection can be used. Electroporation allows transfer of a nucleic acid into a cell by high voltage electric impulse. Additionally, nucleic acids can be introduced into host cells by protoplast fusion, using methods well known in the art. Suitable methods for transforming eukaryotic cells, such as electroporation and lipofection, also are known.

“Host cells” or “Recipient cells” encompassed by of the invention are any cells in which the nucleic acids of the invention can be used to express the polypeptides of the invention. The term also includes any progeny of a recipient or host cell. Preferred recipient or host cells of the invention include E. coli, S. aureus and P. aeruginosa, although other Gram-negative and Gram-positive bacterial, fungal and mammalian cells and organ isms known in the art can be utilized as long as the expression vectors contain an origin of replication to permit expression in the host.

The cationic peptide polynucleotide sequence used according to the method of the invention can be isolated from an organism or syrithesized in the laboratory. Specific DNA sequences encoding the cationic peptide of interest can be obtained by: 1) isolation of a double-stranded DNA sequence from the genomic DNA; 2) chemical manufacture of a DNA sequence to provide the necessary codons for the cationic peptide of interest; and 3) in vitro synthesis of a double-stranded DNA sequence by reverse transcription of mRNA isolated from a donor cell. In the latter case, a double-stranded DNA complement of mRNA is eventually fo)rmed which is generally referred to as cDNA.

The synthesis of DNA sequences is frequently the method of choice when the entire sequence of amino acid residues of the desired peptide product is known. In the present invention, the synthesis of a DNA sequence has the advantage of allowing the incorporation of codons which are more likely to be recognized by a bacterial host, thereby permitting high level expression without difficulties in translation. In addition, virtually any peptide can be synthesized, including those encoding natural cationic peptides, variants of the same, or synthetic peptides.

When the entire sequence of the desired peptide is not known, the direct synthesis of DNA sequences is not possible and the method of choice is the formation of cDNA sequences. Among the standard procedures for isolating cDNA sequences of interest is the formation of plasmid or phage containing cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the cationic peptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single stranded form (Jay, et al., Nuc. Acid Res., 11:2325, 1983).

The peptide of the invention can be administered parenterally by injection or by gradual infusion over time. Preferably the peptide is administered in a therapeutically effective amount to enhance or to stimulate an innate immune response. Innate immunity has been described herein, however examples of indicators of stimulation of innate immunity include but are not limited to monocyte activation, proliferation, differentiation or MAP kinase pathway activation.

The peptide can be administered intravenously, intraperitoneally, intramuscularly, subcutaneously, intracavity, or transdermally. Preferred methods for delivery of the peptide include orally, by encapsulation in microspheres or proteinoids, by aerosol delivery to the lungs, or transdermally by iontophoresis or transdermal electroporation. Other methods of administration will be known to those skilled in the art.

Preparations for parenteral administration of a peptide of the invention include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

In one embodiment, the invention provides a method for synergistic therapy. For example, peptides as described herein can be used in synergistic combination with sub-inhibitory concentrations of antibiotics. Examples of particular classes of antibiotics useful for synergistic therapy with the peptides of the invention include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines and macrolides (e.g., erythromycin and clarithromycin). Further to the antibiotics listed above, typical antibiotics include aminoglycosides (amikacin, gentamicin, kanamycin, netilmicin, tobramycin, s-treptomycin, azithromycin, clarithromycin, erythromycin, erythromycin estolate/ethyl-succinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefiuroxime, cefonicid, cefmnetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin). Other classes of antibiotics include carbapenems (e.g., imipenem), monobactams (e.g.,aztreonam), quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, teicoplanin), for example. Other antibiotics include chloramphenicol, clindamycin, trimethoprim, sulfamethoxazole, nitrofurantoin, rifampin, mupirocin and the cationic peptides.

The efficacy of peptides was evaluated therapeutically alone and in combination with sub-optimal concentrations of antibiotics in models of infection. S. aureus is an important Gram positive pathogen and a leading cause of antibiotic resistant infections. Briefly, peptides were tested for therapeutic efficacy in the S. aureus infection model by injecting them alone and in combination with sub-optimal doses of antibiotics 6 hours after the onset of infection. This would simulate the circumstances of antibiotic resistance developing during an infection, such that the MIC of the resistant bacterium was too high to permit successful therapy (i.e the antibiotic dose applied was sub-optimal). It was demonstrated that the combination of antibiotic and peptide resulted in improved efficacy and suggests the potential for combination therapy (see Example 12).

The invention will now be described in greater detail by reference to the following non-limiting examples. While the invention has been described in detail with reference to certain preferred embodiments thereof, it will be understood that modifications and variations are within the spirit and scope of that which is described and claimed.

### EXAMPLE 1

### Anti-Sepsis/Anti-Inflammatory Activity

Polynucleotide arrays were utilized to determine the effect of cationic peptides on the transcriptional response of epithelial cells. The A549 human epithelial cell line was maintained in DMEM (Gibco) supplemented with 10% fetal bovine serum (FBS, Medicorp). The A549 cells were plated in 100 mm tissue culture dishes at 2.5×10⁶ cells/dish, cultured overnight and then incubated with 100 ng/ml E.coli O111:B4 LPS (Sigma), without (control) or with 50 μg/ml peptide or medium alone for 4 h. After stimulation, the cells were washed once with diethyl pyrocarbonate-treated phosphate buffered saline (PBS), and detached from the dish using a cell scraper. Total RNA was isolated using RNAqueous (Ambion, Austin, Tex.). The RNA pellet was resuspended in RNase-free water containing Superase-In (RNase inhibitor; Ambion). DNA contamination was removed with DNA-free kit, Ambion). The quality of the RNA was assessed by gel electrophoresis on a 1% agarose gel.

The polynucleotide arrays used were the Human Operon arrays (identification number for the genome is PRHU04-S1), which consist of about 14,000 human oligos spotted in duplicate. Probes were prepared from 10 μg of total RNA and labeled with Cy3 or Cy5 labeled dUTP. The probes were purified and hybridized to printed glass slides overnight at 42° C. and washed. After washing, the image was captured using a Perkin Elmer array scanner. The image processing software (Imapolynucleotide 5.0, Marina Del Rey, Calif.) determines the spot mean intensity, median intensities, and background intensities. A “homemade” program was used to remove background. The program calculates the bottom 10% intensity for each subgrid and subtracts this for each grid. Analysis was performed with Genespring software (Redwood City, Calif.). The intensities for each spot were normalized by taking the median spot intensity value from the population of spot values within a slide and comparing this value to the values of all slides in the experiment. The relative changes seen with cells treated with peptide compared to control cells can be found in Tables 1 and 2. These tables reflect only those polynucleotides that demonstrated significant changes in expression of the 14,000 polynucleotides that were tested for altered expression. The data indicate that the peptides have a widespread ability to reduce the expression of polynucleotides that were induced by LPS.

In Table 1, the peptide, SEQ ID NO: 27 is shown to potently reduce the expression of many of the polynucleotides up-regulated by E. coli O1111:B4 LPS as studied by polynucleotide microarrays. Peptide (50 μg/ml) and LPS (0.1 μg/ml) or LPS alone was incubated with the A549 cells for 4 h and the RNA was isolated. Five μg total RNA was used to make Cy3/Cy5 labeled cDNA probes and hybridized onto Human Operon arrays (PRHU04). The intensity of unstimulated cells is shown in the third column of Table 1. The “Ratio: LPS/control” column refers to the intensity of polynucleotide expression in LPS simulated cells divided by in the intensity of unstimulated cells. The “Ratio: LPS+ID 27/control” column refers to the intensity of polynucleotide expression in cells stimulated with LPS and peptide divided by unstimulated cells.

TABLE 1
Reduction, by peptide SEQ ID 27, of A549 human epithelial cell polynucleotide
expression up-regulated by E. coli O111:B4 LPS

| | | Control: | | |
|---|---|---|---|---|
| Accession | Polynucleotide | Media only | Ratio: | Ratio: LPS + ID |
| Number^{a} | Gene Function | Intensity | LPS/control | 27/control |
| AL031983 | Unknown | 0.032 | 302.8 | 5.1 |
| L04510 | ADP- | 0.655 | 213.6 | 1.4 |
| | ribosylation | | | |
| | factor | | | |
| D87451 | ring finger | 3.896 | 183.7 | 2.1 |
| | protein 10 | | | |
| AK000869 | hypothetical | 0.138 | 120.1 | 2.3 |
| | protein | | | |
| U78166 | Ric-like | 0.051 | 91.7 | 0.2 |
| | expressed in | | | |
| | neurons | | | |
| AJ001403 | mucin 5 subtype B | 0.203 | 53.4 | 15.9 |
| | tracheobronchial | | | |
| AB040057 | serine/threonine | 0.95 | 44.3 | 15.8 |
| | protein kinase | | | |
| | MASK | | | |
| Z99756 | Unknown | 0.141 | 35.9 | 14.0 |
| L42243 | interferon | 0.163 | 27.6 | 5.2 |
| | receptor 2 | | | |
| NM_016216 | RNA lariat | 6.151 | 22.3 | 10.9 |
| | debranching | | | |
| | enzyme | | | |
| AK001589 | hypothetical | 0.646 | 19.2 | 1.3 |
| | protein | | | |
| AL137376 | Unknown | 1.881 | 17.3 | 0.6 |
| AB007856 | FEM-1-like | 2.627 | 15.7 | 0.6 |
| | death receptor | | | |
| | binding protein | | | |
| AB007854 | growth arrest- | 0.845 | 14.8 | 2.2 |
| | specific 7 | | | |
| AK000353 | cytosolic ovarian | 0.453 | 13.5 | 1.0 |
| | carcinoma | | | |
| | antigen 1 | | | |
| D14539 | myeloid/lymphoid | 2.033 | 11.6 | 3.1 |
| | or mixed- | | | |
| | lineage leukemia | | | |
| | translocated to 1 | | | |
| X76785 | integration site | 0.728 | 11.6 | 1.9 |
| | for Epstein-Barr | | | |
| | virus | | | |
| M54915 | pim-1 oncogene | 1.404 | 11.4 | 0.6 |
| NM_006092 | caspase | 0.369 | 11.0 | 0.5 |
| | recruitment | | | |
| | domain 4 | | | |
| J03925 | integrin_alpha M | 0.272 | 9.9 | 4.2 |
| NM_001663 | ADP- | 0.439 | 9.7 | 1.7 |
| | ribosylation | | | |
| | factor 6 | | | |
| M23379 | RAS p21 protein | 0.567 | 9.3 | 2.8 |
| | activator | | | |
| K02581 | thymidine kinase | 3.099 | 8.6 | 3.5 |
| | 1 soluble | | | |
| U94831 | transmembrane 9 | 3.265 | 7.1 | 1.5 |
| | superfamily | | | |
| | member 1 | | | |
| X70394 | zinc finger | 1.463 | 6.9 | 1.7 |
| | protein 146 | | | |
| AL137614 | hypothetical | 0.705 | 6.8 | 1.0 |
| | protein | | | |
| U43083 | guanine | 0.841 | 6.6 | 1.6 |
| | cription factor 1 | | | |
| X56777 | zona pellucida | 1.414 | 5.0 | 1.4 |
| | glycoprotein 3A | | | |
| NM_013400 | replication | 1.241 | 4.9 | 2.0 |
| | initiation region | | | |
| | protein | | | |
| NM_002309 | leukemia | 1.286 | 4.8 | 1.9 |
| | inhibitory factor | | | |
| NM_001940 | dentatorubral- | 2.034 | 4.7 | 1.2 |
| | pallidoluysian | | | |
| | atrophy | | | |
| U91316 | cytosolic acyl | 2.043 | 4.7 | 1.4 |
| | coenzyme A | | | |
| | thioester | | | |
| | hydrolase | | | |
| X76104 | death-associated | 1.118 | 4.6 | 1.8 |
| | protein kinase 1 | | | |
| AF131838 | Unknown | 1.879 | 4.6 | 1.4 |
| AL050348 | Unknown | 8.502 | 4.4 | 1.7 |
| D42085 | KIAA0095 gene | 1.323 | 4.4 | 1.2 |
| | product | | | |
| X92896 | Unknown | 1.675 | 4.3 | 1.5 |
| U26648 | syntaxin 5A | 1.59 | 4.3 | 1.4 |
| X85750 | monocyte to | 1.01 | 4.3 | 1.1 |
| | macrophage | | | |
| | differentiation- | | | |
| | associated | | | |
| D14043 | CD164 antigen_sialomucin | 1.683 | 4.2 | 1.0 |
| J04513 | fibroblast growth | 1.281 | 4.0 | 0.9 |
| | factor 2 | | | |
| U19796 | melanoma- | 1.618 | 4.0 | 0.6 |
| | associated | | | |
| | antigen | | | |
| AK000087 | hypothetical | 1.459 | 3.9 | 1.0 |
| | protein | | | |
| AK001569 | hypothetical | 1.508 | 3.9 | 1.2 |
| | protein | | | |
| AF189009 | ubiquilin 2 | 1.448 | 3.8 | 1.3 |
| U60205 | sterol-C4-methyl | 1.569 | 3.7 | 0.8 |
| | oxidase-like | | | |
| AK000562 | hypothetical | 1.166 | 3.7 | 0.6 |
| | protein | | | |
| AL096739 | Unknown | 3.66 | 3.7 | 0.5 |
| AK000366 | hypothetical | 15.192 | 3.5 | 1.0 |
| | protein | | | |
| NM_006325 | RAN member | 1.242 | 3.5 | 1.4 |
| | RAS oncogene | | | |
| | family | | | |
| X51688 | cyclin A2 | 1.772 | 3.3 | 1.0 |
| U34252 | aldehyde | 1.264 | 3.3 | 1.2 |
| | dehydrogenase 9 | | | |
| NM_013241 | FH1/FH2 | 1.264 | 3.3 | 0.6 |
| | domain- | | | |
| | containing | | | |
| | protein | | | |
| AF112219 | esterase | 1.839 | 3.3 | 1.1 |
| | D/formylglutathi | | | |
| | one hydrolase | | | |
| NM_016237 | anaphase- | 2.71 | 3.2 | 0.9 |
| | promoting | | | |
| | complex subunit 5 | | | |
| AB014569 | KIAA0669 gene | 2.762 | 3.2 | 0.2 |
| | product | | | |
| AF151047 | hypothetical | 3.062 | 3.1 | 1.0 |
| | protein | | | |
| X92972 | protein | 2.615 | 3.1 | 1.1 |
| | phosphatase 6 | | | |
| | catalytic subunit | | | |
| AF035309 | proteasome 26S | 5.628 | 3.1 | 1.3 |
| | subunit ATPase 5 | | | |
| U52960 | SRB7 homolog | 1.391 | 3.1 | 0.8 |
| J04058 | electron-transfer- | 3.265 | 3.1 | 1.2 |
| | flavoprotein | | | |
| | alpha | | | |
| | polypeptide | | | |
| M57230 | interleukin 6 | 0.793 | 3.1 | 1.0 |
| | signal transducer | | | |
| U78027 | galactosidase_alpha | 3.519 | 3.1 | 1.1 |
| AK000264 | Unknown | 2.533 | 3.0 | 0.6 |
| X80692 | mitogen- | 2.463 | 2.9 | 1.3 |
| | activated protein | | | |
| | kinase 6 | | | |
| L25931 | lamin B receptor | 2.186 | 2.7 | 0.7 |
| X13334 | CD14 antigen | 0.393 | 2.5 | 1.1 |
| M32315 | tumor necrosis | 0.639 | 2.4 | 0.4 |
| | factor receptor | | | |
| | superfamily | | | |
| | member 1B | | | |
| NM_004862 | LPS-induced | 6.077 | 2.3 | 1.1 |
| | TNF-alpha factor | | | |
| AL050337 | interferon | 2.064 | 2.1 | 1.0 |
| | gamma receptor 1 | | | |

^{a}All Accession Numbers in Table 1 through Table 64 refer to GenBank Accession Numbers.

In Table 2, the cationic peptides at a concentration of 50 μg/ml were shown to potently reduce the expression of many of the polynucleotides up-regulated by 100 ng/ml E. coli O111:B4 LPS as studied by polynucleotide microarrays. Peptide and LPS or LPS alone was incubated with the A549 cells for 4 h and the RNA was isolated. 5 μg total RNA was used to make Cy3/Cy5 labeled cDNA probes and hybridized onto Human Operon arrays (PRHU04). The intensity of unstimulated cells is shown in the third column of Table 2. The “Ratio: LPS/control” column refers to the intensity of polynucleotide expression in LPS-simulated cells divided by in the intensity of unstimulated cells. The other columns refer to the intensity of polynucleotide expression in cells stimulated with LPS and peptide divided by unstimulated cells.

TABLE 2
Human A549 Epithelial Cell Polynucleotide Expression up-regulated
by E. coli O111:B4 LPS and reduced by Cationic Peptides.

| | | Ctrl:Media | | Ratio: | Ratio: | Ratio: |
|---|---|---|---|---|---|---|
| Accession | | only | Ratio: | LPS + ID | LPS + ID | LPS + ID |
| Number | Gene | Intensity | LPS/Ctrl | 27/Ctrl | 16/Ctrl | 22/Ctrl |
| AL031983 | Unknown | 0.03 | 302.8 | 5.06 | 6.91 | 0.31 |
| L04510 | ADP- | 0.66 | 213.6 | 1.4 | 2.44 | 3.79 |
| | ribosylation | | | | | |
| | factor | | | | | |
| D87451 | ring finger | 3.90 | 183.7 | 2.1 | 3.68 | 4.28 |
| | protein | | | | | |
| AK000869 | hypothetical | 0.14 | 120.1 | 2.34 | 2.57 | 2.58 |
| | protein | | | | | |
| U78166 | Ric like | 0.05 | 91.7 | 0.20 | 16.88 | 21.37 |
| X03066 | MHC class II | 0.06 | 36.5 | 4.90 | 12.13 | 0.98 |
| | DO beta | | | | | |
| AK001904 | hypothetical | 0.03 | 32.8 | 5.93 | 0.37 | 0.37 |
| | protein | | | | | |
| AB037722 | Unknown | 0.03 | 21.4 | 0.30 | 0.30 | 2.36 |
| AK001589 | hypothetical | 0.65 | 19.2 | 1.26 | 0.02 | 0.43 |
| | protein | | | | | |
| AL137376 | Unknown | 1.88 | 17.3 | 0.64 | 1.30 | 1.35 |
| L19185 | thioredoxin- | 0.06 | 16.3 | 0.18 | 2.15 | 0.18 |
| | dependent per- | | | | | |
| | oxide reductase 1 | | | | | |
| J05068 | transcobalamin 1 | 0.04 | 15.9 | 1.78 | 4.34 | 0.83 |
| AB007856 | FEM-1-like | 2.63 | 15.7 | 0.62 | 3.38 | 0.96 |
| | death receptor | | | | | |
| | binding protein | | | | | |
| AK000353 | cytosolic | 0.45 | 13.5 | 1.02 | 1.73 | 2.33 |
| | ovarian | | | | | |
| | carcinoma ag 1 | | | | | |
| X16940 | smooth muscle | 0.21 | 11.8 | 3.24 | 0.05 | 2.26 |
| | enteric actin γ2 | | | | | |
| M54915 | pim-1 oncogene | 1.40 | 11.4 | 0.63 | 1.25 | 1.83 |
| AL122111 | hypothetical | 0.37 | 10.9 | 0.21 | 1.35 | 0.03 |
| | protein | | | | | |
| M95678 | phospholipase | 0.22 | 7.2 | 2.38 | 0.05 | 1.33 |
| | C beta 2 | | | | | |
| AK001239 | hypothetical | 2.20 | 6.4 | 1.27 | 1.89 | 2.25 |
| | protein | | | | | |
| AC004849 | Unknown | 0.14 | 6.3 | 0.07 | 2.70 | 0.07 |
| X06614 | retinoic acid | 1.92 | 5.5 | 0.77 | 1.43 | 1.03 |
| | receptor_alpha | | | | | |
| AB007896 | putative L-type | 0.94 | 5.3 | 1.82 | 2.15 | 2.41 |
| | neutral amino | | | | | |
| | acid transporter | | | | | |
| AB010894 | BAI1- | 0.69 | 5.0 | 1.38 | 1.03 | 1.80 |
| | associated | | | | | |
| | protein | | | | | |
| U52522 | partner of | 1.98 | 2.9 | 1.35 | 0.48 | 1.38 |
| | RAC1 | | | | | |
| AK001440 | hypothetical | 1.02 | 2.7 | 0.43 | 1.20 | 0.01 |
| | protein | | | | | |
| NM_001148 | ankyrin 2_neuronal | 0.26 | 2.5 | 0.82 | 0.04 | 0.66 |
| X07173 | inter-alpha | 0.33 | 2.2 | 0.44 | 0.03 | 0.51 |
| | inhibitor H2 | | | | | |
| AF095687 | brain and | 0.39 | 2.1 | 0.48 | 0.03 | 0.98 |
| | nasopharyngeal | | | | | |
| | carcinoma | | | | | |
| | susceptibility | | | | | |
| | protein | | | | | |
| NM_016382 | NK cell | 0.27 | 2.1 | 0.81 | 0.59 | 0.04 |
| | activation | | | | | |
| | inducing ligand | | | | | |
| | NAIL | | | | | |
| AB023198 | KIAA0981 | 0.39 | 2.0 | 0.43 | 0.81 | 0.92 |
| | protein | | | | | |

### EXAMPLE 2

### Neutralization of the Stimulation of Immune Cells

The ability of compounds to neutralize the stimulation of immune cells by both Gram-negative and Gram-positive bacterial products was tested. Bacterial products stimulate cells of the immune system to produce inflammatory cytokines and when unchecked this can lead to sepsis. Initial experiments utilized the murine macrophage cell line RAW 264.7, which was obtained from the American Type Culture Collection, (Manassas, Va.), the human epithelial cell line, A549, and primary macrophages derived from the bone marrow of BALB/c mice (Charles River Laboratories, Wilmington, Mass.). The cells from mouse bone marrow were cultured in 150-mm plates in Dulbecco's modified Eagle medium (DMEM; Life Technologies, Burlington, ON) supplemented with 20% FBS (Sigma Chemical Co, St. Louis, Mo.) and 20% L cell-conditioned medium as a source of M-CSF. Once macrophages were 60-80% confluent, they were deprived of L cell-conditioned medium for 14-16 h to render the cells quiescent and then were subjected to treatments with 100 ng/ml LPS or 100 ng/ml LPS+20 μg/ml peptide for 24 hours. The release of cytokines into the culture supernatant was determined by ELISA (R&D Systems, Minneapolis, Minn.). The cell lines, RAW 264.7 and A549, were maintained in DMEM supplemented with 10% fetal calf serum. RAW 264.7 cells were seeded in 24 well plates at a density of 10⁶ cells per well in DMEM and A549 cells were seeded in 24 well plates at a density of 10⁵ cells per well in DMEM and both were incubated at 37° C. in 5% CO₂ overnight. DMEM was aspirated from cells grown overnight and replaced with fresh medium. In some experiments, blood from volunteer human donors was collected (according to procedures accepted by UBC Clinical Research Ethics Board, certificate C00-0537) by venipuncture into tubes (Becton Dickinson, Franklin Lakes, N.J.) containing 14.3 USP units heparin/ml blood. The blood was mixed with LPS with or without peptide in polypropylene tubes at 37° C. for 6 h. The samples were centrifuged for 5 min at 2000×g, the plasma was collected and then stored at −20° C. until being analyzed for IL-8 by ELISA (R&D Systems). In the experiments with cells, LPS or other bacterial products were incubated with the cells for 6-24 hr at 37° C. in 5% CO₂. S. typhimurium LPS and E. coli 0111:B4 LPS were purchased from Sigma. Lipoteichoic acid (LTA) from S. aureus (Sigma) was resuspended in endotoxin free water (Sigma). The Limulus amoebocyte lysate assay (Sigma) was performed on LTA preparations to confirm that lots were not significantly contaminated by endotoxin. Endotoxin contamination was less than 1 ng/ml, a concentration that did not cause significant cytokine production in the RAW 264.7 cells. Non-capped lipoarabinomannan (AraLAM ) was a gift from Dr. John T. Belisle of Colorado State University. The AraLAM from Mycobacterium was filter sterilized and the endotoxin contamination was found to be 3.75 ng per 1.0 mg of LAM as determined by Limulus Amebocyte assay. At the same time as LPS addition (or later where specifically described), cationic peptides were added at a range of concentrations. The supernatants were removed and tested for cytokine production by ELISA (R&D Systems). All assays were performed at least three times with similar results. To confirm the anti-sepsis activity in vivo, sepsis was induced by intraperitoneal injection of 2 or 3 μg of E. coli O111:B4 LPS in phosphate-buffered saline (PBS; pH 7.2) into galactosamine-sensitized 8- to 10-week-old female CD-1 or BALB/c mice. In experiments involving peptides, 200 μg in 100 μl of sterile water was injected at separate intraperitoneal sites within 10 min of LPS injection. In other experiments, CD-1 mice were injected with 400 μg E. coli 011 I :B4 LPS and 10 min later peptide (200 μg) was introduced by intraperitoneal injection. Survival was monitored for 48 hours post injection.

Hyperproduction of TNF-α has been classically linked to development of sepsis. The three types of LPS, LTA or AraLAM used in this example represented products released by both Gram-negative and Gram-positive bacteria. Peptide, SEQ ID NO: 1, was able to significantly reduce TNF-A production stimulated by S. typhimurium, B. cepacia, and E. coli O111:B4 LPS, with the former being affected to a somewhat lesser extent (Table 3). At concentrations as low as 1 μg/ml of peptide (0.25 nM) substantial reduction of TNF-α production was observed in the latter two cases. A different peptide, SEQ ID NO: 3 did not reduce LPS-induced production of TNF-α in RAW macrophage cells, demonstrating that this is not a uniform and predictable property of cationic peptides. Representative peptides from each Formula were also tested for their ability to affect TNF-α production stimulated by E. coli O111:B4 LPS (Table 4). The peptides had a varied ability to reduce TNF-α production although many of them lowered TNF-α by at least 60%.

At certain concentrations peptides SEQ ID NO: 1 and SEQ ID NO: 2, could also reduce the ability of bacterial products to stimulate the production of IL-8 by an epithelial cell line. LPS is a known potent stimulus of IL-8 production by epithelial cells. Peptides, at low concentrations (1-20 μg/ml), neutralized the IL-8 induction responses of epithelial cells to LPS (Tables 5-7). Peptide SEQ ID 2 also inhibited LPS-induced production of IL-8 in whole human blood (Table 4). Conversely, high concentrations of peptide SEQ ID NO: 1 (50 to 100 μg/ml) actually resulted in increased levels of IL-8 (Table 5). This suggests that the peptides have different effects at different concentrations.

The effect of peptides on inflammatory stimuli was also demonstrated in primary murine. cells, in that peptide SEQ ID NO: 1 significantly reduced TNF-α production (>90%) by bone marrow-derived macrophages from BALB/c mice that had been stimulated with 100 ng/ml E. coli 0111 :B4 LPS (Table 8). These experiments were performed in the presence of serum, which contains LPS-binding protein (LBP), a protein that can mediate the rapid binding of LPS to CD14. Delayed addition of SEQ ID NO: 1 to the supernatants of macrophages one hour after stimulation with 100 ng/ml E. coli LPS still resulted in substantial reduction (70%) of TNF-α production (Table 9).

Consistent with the ability of SEQ ID NO: 1 to prevent LPS-induced production of TNF-α in vitro, certain peptides also protected mice against lethal shock induced by high concentrations of LPS. In some experiments, CD-1 mice were sensitized to LPS with a prior injection of galactosamine. Galactosamine-sensitized mice that were injected with 3 μg of E. coli 0111:B4 LPS were all killed within 4-6 hours. When 200 μg of SEQ ID NO: 1 was injected 15 min after the LPS, 50% of the mice survived (Table 10). In other experiments when a higher concentration of LPS was injected into BALB/c mice with no D-galactosamine, peptide protected 100% compared to the control group in which there was no survival (Table 13). Selected other peptides were also found to be protective in these models (Tables 11,12).

Cationic peptides were also able to lower the stimulation of macrophages by Gram-positive bacterial products such as Mycobacterium non-capped lipoarabinomannan (AraLAM) and S. aureus LTA. For example, SEQ ID NO: 1 inhibited induction of TNF-α in RAW 264.7 cells by the Gram-positive bacterial products, LTA (Table 14) and to a lesser extent AraLAM (Table 15). Another peptide, SEQ ID NO: 2, was also found to reduce LTA-induced TNF-α production by RAW 264.7 cells. At a concentration of 1 μg/ml SEQ ID NO: 1 was able to substantially reduce (>75%) the induction of TNF-α production by 1 μg/ml S. aureus LTA. At 20 μg/ml SEQ ID NO: 1, there was >60% inhibition of AraLAM induced TNF-α. Polymyxin B (PMB) was included as a control to demonstrate that contaminating endotoxin was not a significant factor in the inhibition by SEQ ID NO: 1 of AraLAM induced TNF-α. These results demonstrate that cationic peptides can reduce the pro-inflammatory cytokine response of the immune system to bacterial products.

TABLE 3
Reduction by SEQ ID 1 of LPS induced TNF-α
production in RAW 264.7 cells.

| Amount of SEQ | Inhibition off TNF-α (%)* | | |
|---|---|---|---|
| ID NO: 1 (μg/ml) | B. cepacia LPS | E. coli LPS | S. typhimurium LPS |
| 0.1 | 8.5 ± 2.9 | 0.0 ± 0.6 | 0.0 ± 0 |
| 1 | 23.0 ± 11.4 | 36.6 ± 7.5 | 9.8 ± 6.6 |
| 5 | 55.4 ± 8 | 65.0 ± 3.6 | 31.1 ± 7.0 |
| 10 | 63.1 ± 8 | 75.0 ± 3.4 | 37.4 ± 7.5 |
| 20 | 71.7 ± 5.8 | 81.0 ± 3.5 | 58.5 ± 10.5 |
| 50 | 86.7 ± 4.3 | 92.6 ± 2.5 | 73.1 ± 9.1 |
| RAW 264.7 mouse macrophage cells were stimulated with 100 ng/ml S. typhimurium LPS, 100 ng/ml B. cepacia LPS and 100 ng/ml E. coli 0111:B4 LPS in the presence of the indicated concentrations of SEQ ID 1 for 6 hr. The concentrations of TNF-α released into the culture supernatants were determined by ELISA. 100% represents the amount of TNF-α resulting from RAW 264.7 cells incubated with LPS alone for 6 hours (S. typhimurium LPS = 34.5 ± 3.2 ng/ml, | | | |
| # B. cepacia LPS = 11.6 ± 2.9 ng/ml, and E. coli 0111:B4 LPS = 30.8 ± 2.4 ng/ml). Background levels of TNF-α production by the RAW 264.7 cells cultured with no stimuli for 6 hours resulted in TNF-α levels ranging from 0.037-0.192 ng/ml. The data is from duplicate samples and presented as the mean of three experiments + standard error. | | | |

TABLE 4
Reduction by Cationic Peptides of E. coli LPS
induced TNF-α production in RAW 264.7 cells.

| Peptide (20 μg/ml) | Inhibition of TNF-α (%) |
|---|---|
| SEQ ID NO: 5 | 65.6 ± 1.6 |
| SEQ ID NO: 6 | 59.8 ± 1.2 |
| SEQ ID NO: 7 | 50.6 ± 0.6 |
| SEQ ID NO: 8 | 39.3 ± 1.9 |
| SEQ ID NO: 9 | 58.7 ± 0.8 |
| SEQ ID NO: 10 | 55.5 ± 0.52 |
| SEQ ID NO: 12 | 52.1 ± 0.38 |
| SEQ ID NO: 13 | 62.4 ± 0.85 |
| SEQ ID NO: 14 | 50.8 ± 1.67 |
| SEQ ID NO: 15 | 69.4 ± 0.84 |
| SEQ ID NO: 16 | 37.5 ± 0.66 |
| SEQ ID NO: 17 | 28.3 ± 3.71 |
| SEQ ID NO: 19 | 69.9 ± 0.09 |
| SEQ ID NO: 20 | 66.1 ± 0.78 |
| SEQ ID NO: 21 | 67.8 ± 0.6 |
| SEQ ID NO: 22 | 73.3 ± 0.36 |
| SEQ ID NO: 23 | 83.6 ± 0.32 |
| SEQ ID NO: 24 | 60.5 ± 0.17 |
| SEQ ID NO: 26 | 54.9 ± 1.6 |
| SEQ ID NO: 27 | 51.1 ± 2.8 |
| SEQ ID NO: 28 | 56 ± 1.1 |
| SEQ ID NO: 29 | 58.9 ± 0.005 |
| SEQ ID NO: 31 | 60.3 ± 0.6 |
| SEQ ID NO: 33 | 62.1 ± 0.08 |
| SEQ ID NO: 34 | 53.3 ± 0.9 |
| SEQ ID NO: 35 | 60.7 ± 0.76 |
| SEQ ID NO: 36 | 63 ± 0.24 |
| SEQ ID NO: 37 | 58.9 ± 0.67 |
| SEQ ID NO: 38 | 54 ± 1 |
| SEQ ID NO: 40 | 75 ± 0.45 |
| SEQ ID NO: 41 | 86 ± 0.37 |
| SEQ ID NO: 42 | 80.5 ± 0.76 |
| SEQ ID NO: 43 | 88.2 ± 0.65 |
| SEQ ID NO: 44 | 44.9 ± 1.5 |
| SEQ ID NO: 45 | 44.7 ± 0.39 |
| SEQ ID NO: 47 | 36.9 ± 2.2 |
| SEQ ID NO: 48 | 64 ± 0.67 |
| SEQ ID NO: 49 | 86.9 ± 0.69 |
| SEQ ID NO: 53 | 46.5 ± 1.3 |
| SEQ ID NO: 54 | 64 ± 0.73 |
| RAW 264.7 mouse macrophage cells were stimulated with 100 ng/ml E. coli 0111:B4 LPS in the presence of the indicated concentrations of cationic peptides for 6 h. The concentrations of TNF-α released into the culture supernatants were determined by ELISA. Background levels of TNF-α production by the RAW 264.7 cells cultured with no stimuli for 6 hours resulted in TNF-α levels ranging from 0.037-0.192 ng/ml. The data is from duplicate samples and | |
| # presented as the mean of three experiments + standard deviation. | |

TABLE 5
Reduction by SEQ ID NO: 1 of LPS induced
IL-8 production in A549 cells.

| SEQ ID NO: 1 (μg/ml) | Inhibition of IL-8 (%) |
|---|---|
| 0.1 | 1 ± 0.3 |
| 1 | 32 ± 10 |
| 10 | 60 ± 9 |
| 20 | 47 ± 12 |
| 50 | 40 ± 13 |
| 100 | 0 |

A549 cells were stimulated with increasing concentrations of SEQ ID 1 in the presence of LPS (100 ng/ml E. coli O111:B4) for 24 hours. The concentration of IL-8 in the culture supernatants was determined by ELISA. The background levels of IL-8 from cells alone was 0.172 ± 0.029 ng/ml. The data is presented as the mean of three experiments + standard error.

TABLE 6
Reduction by SEQ ID NO: 2 of E. coli LPS induced
IL-8 production in A549 cells.

| Concentration of SEQ ID NO: 2 (μg/ml) | Inhibition of IL-8 (%) |
|---|---|
| 0.1 | 6.8 ± 9.6 |
| 1 | 12.8 ± 24.5 |
| 10 | 29.0 ± 26.0 |
| 50 | 39.8 ± 1.6 |
| 100 | 45.0 ± 3.5 |

Human A549 epithelial cells were stimulated with increasing concentrations of SEQ ID NO: 2 in the presence of LPS (100 ng/ml E. coli O111:B4) for 24 hours. The concentration of IL-8 in the culture supernatants was determined by ELISA. The data is presented as the mean of three experiments + standard error.

TABLE 7
Reduction by SEQ ID NO: 2 of E. coli LPS
induced IL-8 in human blood.

| SEQ ID NO: 2 (μg/ml) | IL-8 (pg/ml) |
|---|---|
| 0 | 3205 |
| 10 | 1912 |
| 50 | 1458 |

Whole human blood was stimulated with increasing concentrations of peptide and E. coli O111:B4 LPS for 4 hr. The human blood samples were centrifuged and the serum was removed and tested for IL-8 by ELISA. The data is presented as the average of 2 donors.

TABLE 8
Reduction by SEQ ID NO: 1 of E. coli LPS induced
TNF-α production in murine bone marrow macrophages.

Production of
TNF-α (ng/ml)

| SEQ ID NO: 1 (μg/ml) | 6 hours | 24 hours |
|---|---|---|
| LPS alone | 1.1 | 1.7 |
| 1 | 0.02 | 0.048 |
| 10 | 0.036 | 0.08 |
| 100 | 0.033 | 0.044 |
| No LPS control | 0.038 | 0.06 |
| BALB/c Mouse bone marrow-derived macrophages were cultured for either 6 h or 24 h with 100 ng/ml E. coli 0111:B4 LPS in the presence or absence of 20 μg/ml of peptide. The supernatant was collected and tested for levels of TNF-α by ELISA. The data represents the amount of TNF-α resulting from duplicate wells of bone marrow-derived macrophages incubated with LPS alone for 6 h (1.1 ± 0.09 ng/ml) or 24 h (1.7 ± 0.2 ng/ml). Background levels of TNF-α were | | |
| # 0.038 ± 0.008 ng/ml for 6 h and 0.06 ± 0.012 ng/ml for 24 h. | | |

TABLE 9
Inhibition of E. coli LPS-induced TNF-α production
by delayed addition of SEQ ID NO: 1 to A549 cells.

| Time of addition of SEQ ID NO: 1 | |
|---|---|
| after LPS (min) | Inhibition of TNF-α (%) |
| 0 | 98.3 ± 0.3 |
| 15 | 89.3 ± 3.8 |
| 30 | 83 ± 4.6 |
| 60 | 68 ± 8 |
| 90 | 53 ± 8 |

Peptide (20 μg/ml) was added at increasing time points to wells already containing A549 human epithelial cells and 100 ng/ml E. coli 0111:B4 LPS. The supernatant was collected after 6 hours and tested for levels of TNF-α by ELISA. The data is presented as the mean of three experiments ± standard error.

TABLE 10
Protection against lethal endotoxemia in galactosamine-
sensitized CD-1 mice by SEQ ID NO: 1.

| D-Galactosamine | | E. coli | Peptide or | Total | Survival post |
|---|---|---|---|---|---|
| treatment | | 0111:B4 LPS | buffer | mice | endotoxin shock |
| 0 | | 3 μg | PBS | 5 | 5 (100%) |
| 20 | mg | 3 μg | PBS | 12 | 0 (0%) |
| 20 | mg | 3 μg | SEQ ID | 12 | 6 (50%) |
| | | | NO: 1 | | |

CD-1 mice (9 weeks-old) were sensitized to endotoxin by three intraperitoneal injections of galactosamine (20 mg in 0.1 ml sterile PBS). Then endotoxic shock was induced by intraperitoneal injection of E. coli 0111:B4 LPS (3 μg in 0.1 ml PBS). Peptide, SEQ ID NO: 1, (200 μg/mouse = 8 mg/kg) was injected at a separate intraperitoneal site 15 min after injection of LPS. The mice were monitored for 48 hours and the results were recorded.

TABLE 11
Protection against lethal endotoxemia in galactosamine-sensitized
CD-1 mice by Cationic Peptides.

| | E. coli 0111:B4 | Number | Survival |
|---|---|---|---|
| Peptide Treatment | LPS added | of Mice | (%) |
| Control (no peptide) | 2 μg | 5 | 0 |
| SEQ ID NO: 6 | 2 μg | 5 | 40 |
| SEQ ID NO: 13 | 2 μg | 5 | 20 |
| SEQ ID NO: 17 | 2 μg | 5 | 40 |
| SEQ ID NO: 24 | 2 μg | 5 | 0 |
| SEQ ID NO: 27 | 2 μg | 5 | 20 |

CD-1 mice (9 weeks-old) were sensitized to endotoxin by intraperitoneal injection of galactosamine (20 mg in 0.1 ml sterile PBS). Then endotoxic shock was induced by intraperitoneal injection of E. coli 0111:B4 LPS (2 μg in 0.1 ml PBS). Peptide (200 μg/mouse = 8 mg/kg) was injected at a separate intraperitoneal site 15 min after injection of LPS. The mice were monitored for 48 hours and the results were recorded.

TABLE 12
Protection against lethal endotoxemia in galactosamine-sensitized
BALB/c mice by Cationic Peptides.

| | E. coli | Number | Survival |
|---|---|---|---|
| Peptide Treatment | 0111:B4 LPS added | of Mice | (%) |
| No peptide | 2 μg | 10 | 10 |
| SEQ ID NO: 1 | 2 μg | 6 | 17 |
| SEQ ID NO: 3 | 2 μg | 6 | 0 |
| SEQ ID NO: 5 | 2 μg | 6 | 17 |
| SEQ ID NO: 6 | 2 μg | 6 | 17 |
| SEQ ID NO: 12 | 2 μg | 6 | 17 |
| SEQ ID NO: 13 | 2 μg | 6 | 33 |
| SEQ ID NO: 15 | 2 μg | 6 | 0 |
| SEQ ID NO: 16 | 2 μg | 6 | 0 |
| SEQ ID NO: 17 | 2 μg | 6 | 17 |
| SEQ ID NO: 23 | 2 μg | 6 | 0 |
| SEQ ID NO: 24 | 2 μg | 6 | 17 |
| SEQ ID NO: 26 | 2 μg | 6 | 0 |
| SEQ ID NO: 27 | 2 μg | 6 | 50 |
| SEQ ID NO: 29 | 2 μg | 6 | 0 |
| SEQ ID NO: 37 | 2 μg | 6 | 0 |
| SEQ ID NO: 38 | 2 μg | 6 | 0 |
| SEQ ID NO: 41 | 2 μg | 6 | 0 |
| SEQ ID NO: 44 | 2 μg | 6 | 0 |
| SEQ ID NO: 45 | 2 μg | 6 | 0 |
| BALB/c mice (8 weeks-old) were sensitized to endotoxin by intraperitoneal injection of galactosamine (20 mg in 0.1 ml sterile PBS). Then endotoxic shock was induced by intraperitoneal injection of E. coli 0111:B4 LPS (2 μg in 0.1 ml PBS). Peptide (200 μg/mouse = 8 mg/kg) was injected at a separate intraperitoneal site 15 min after injection of LPS. The mice were monitored | | | |
| # for 48 hours and the results were recorded. | | | |

TABLE 13
Protection against lethal endotoxemia in BALB/c mice by SEQ ID
NO: 1.

| | E. coli | Number | |
|---|---|---|---|
| Peptide Treatment | 0111:B4 LPS | of Mice | Survival (%) |
| No peptide | 400 μg | 5 | 0 |
| SEQ ID NO: 1 | 400 μg | 5 | 100 |

BALB/c mice were injected intraperitoneal with 400 μg E. coli 0111:B4 LPS. Peptide (200 μg/mouse = 8 mg/kg) was injected at a separate intraperitoneal site and the mice were monitored for 48 hours and the results were recorded.

TABLE 14
Peptide inhibition of TNF-α production induced by S. aureus LTA.

| SEQ ID NO: 1 added (μg/ml) | Inhibition of TNF-α (%) |
|---|---|
| 0.1 | 44.5 ± 12.5 |
| 1 | 76.7 ± 6.4 |
| 5 | 91 ± 1 |
| 10 | 94.5 ± 1.5 |
| 20 | 96 ± 1 |

RAW 264.7 mouse macrophage cells were stimulated with 1 μg/ml S. aureus LTA in the absence and presence of increasing concentrations of peptide. The supernatant was collected and tested for levels of TNF-α by ELISA. Background levels of TNF-α production by the RAW 264.7 cells cultured with no stimuli for 6 hours resulted in TNF-α levels ranging from 0.037-0.192 ng/ml. The data is presented as the mean of three or more experiments + standard error.

TABLE 15
Peptide inhibition of TNF-α production induced by Mycobacterium
non-capped lipoarabinomannan.

| Peptide (20 μg/ml) | Inhibition of TNF-α (%) |
|---|---|
| No peptide | 0 |
| SEQ ID NO: 1 | 64 ± 5.9 |
| Polymyxin B | 15 ± 2 |
| RAW 264.7 mouse macrophage cells were stimulated with 1 μg/ml AraLAM in the absence and presence of 20 μg/ml peptide or Polymyxin B. The supernatant was collected and tested for levels of TNF-α by ELISA. Background levels of TNF-α production by the RAW 264.7 cells cultured with no stimuli for 6 hours resulted in TNF-α levels ranging from 0.037-0.192 ng/ml. The data is | |
| # presented as the mean inhibition of three or more experiments + standard error. | |

### EXAMPLE 3

### Assessment of Toxicity of the Cationic Peptides

The potential toxicity of the peptides was measured in two ways. First, the Cytotoxicity Detection Kit (Roche) (Lactate dehydrogenase-LDH) Assay was used. It is a colorimetric assay for the quantification of cell death and cell lysis, based on the measurement of LDH activity released from the cytosol of damaged cells into the supernatant. LDH is a stable cytoplasmic enzyme present in all cells and it is released into the cell culture supernatant upon damage of the plasma membrane. An increase in the amount of dead or plasma membrane-damaged cells results in an increase of the LDH enzyme activity in the culture supernatant as measured with an ELISA plate reader, OD₄₉₀ nm (the amount of color formed in the assay is proportional to the number of lysed cells). In this assay, human bronchial epithelial cells (I6HBEo14, HBE) cells were incubated with 100 μg of peptide for 24 hours, the supernatant removed and tested for LDH. The other assay used to measure toxicity of the cationic peptides was the WST-1 assay (Roche). This assay is a colorimetric assay for the quantification of cell proliferation and cell viability, based on the cleavage of the tetrazolium salt WST-1 by mitochondrial dehydrogenases in viable cells (a non-radioactive alternative to the [³H]-thymidine incorporation assay). In this assay, HBE cells were incubated with 100 μg of peptide for 24 hours, and then 10 μl/well Cell Proliferation Reagent WST-1 was added. The cells are incubated with the reagent and the plate is then measured with an ELISA plate reader, OD₄₉₀ nm.

The results shown below in Tables 16 and 17 demonstrate that most of the peptides are not toxic to the cells tested. However, four of the peptides from Formula F (SEQ ID NOS: 40, 41, 42 and 43) did induce membrane damage as measured by both assays.

TABLE 16
Toxicity of the Cationic Peptides as Measured by the LDH Release
Assay.

| Treatment | LDH Release (OD₄₉₀ nm) |
|---|---|
| No cells Control | 0.6 ± 0.1 |
| Triton X-100 Control | 4.6 ± 0.1 |
| No peptide control | 1.0 ± 0.05 |
| SEQ ID NO: 1 | 1.18 ± 0.05 |
| SEQ ID NO: 3 | 1.05 ± 0.04 |
| SEQ ID NO: 6 | 0.97 ± 0.02 |
| SEQ ID NO: 7 | 1.01 ± 0.04 |
| SEQ ID NO: 9 | 1.6 ± 0.03 |
| SEQ ID NO: 10 | 1.04 ± 0.04 |
| SEQ ID NO: 13 | 0.93 ± 0.06 |
| SEQ ID NO: 14 | 0.99 ± 0.05 |
| SEQ ID NO: 16 | 0.91 ± 0.04 |
| SEQ ID NO: 17 | 0.94 ± 0.04 |
| SEQ ID NO: 19 | 1.08 ± 0.02 |
| SEQ ID NO: 20 | 1.05 ± 0.03 |
| SEQ ID NO: 21 | 1.06 ± 0.04 |
| SEQ ID NO: 22 | 1.29 ± 0.12 |
| SEQ ID NO: 23 | 1.26 ± 0.46 |
| SEQ ID NO: 24 | 1.05 ± 0.01 |
| SEQ ID NO: 26 | 0.93 ± 0.04 |
| SEQ ID NO: 27 | 0.91 ± 0.04 |
| SEQ ID NO: 28 | 0.96 ± 0.06 |
| SEQ ID NO: 29 | 0.99 ± 0.02 |
| SEQ ID NO: 31 | 0.98 ± 0.03 |
| SEQ ID NO: 33 | 1.03 ± 0.05 |
| SEQ ID NO: 34 | 1.02 ± 0.03 |
| SEQ ID NO: 35 | 0.88 ± 0.03 |
| SEQ ID NO: 36 | 0.85 ± 0.04 |
| SEQ ID NO: 37 | 0.96 ± 0.04 |
| SEQ ID NO: 38 | 0.95 ± 0.02 |
| SEQ ID NO: 40 | 2.8 ± 0.5 |
| SEQ ID NO: 41 | 3.3 ± 0.2 |
| SEQ ID NO: 42 | 3.4 ± 0.2 |
| SEQ ID NO: 43 | 4.3 ± 0.2 |
| SEQ ID NO: 44 | 0.97 ± 0.03 |
| SEQ ID NO: 45 | 0.98 ± 0.04 |
| SEQ ID NO: 47 | 1.05 ± 0.05 |
| SEQ ID NO: 48 | 0.95 ± 0.05 |
| SEQ ID NO: 53 | 103 ± 0.06 |
| Polymyxin B | 1.21 ± 0.03 |

Human HBE bronchial epithelial cells were incubated with 100 μg/ml peptide or Polymyxin B for 24 hours. LDH activity was assayed in the supernatant of the cell cultures. As a control for 100% LDH release, Triton X-100 was added. The data is presented as the mean ± standard deviation. Only peptides SEQ ID 40, 41, 42 and 43 showed any significant toxicity.

TABLE 17
Toxicity of the Cationic Peptides as Measured by the WST-1 Assay.

| Treatment | OD₄₉₀ nm |
|---|---|
| No cells Control | 0.24 ± 0.01 |
| Triton X-100 Control | 0.26 ± 0.01 |
| No peptide control | 1.63 ± 0.16 |
| SEQ ID NO: 1 | 1.62 ± 0.34 |
| SEQ ID NO: 3 | 1.35 ± 0.12 |
| SEQ ID NO: 10 | 1.22 ± 0.05 |
| SEQ ID NO: 6 | 1.81 ± 0.05 |
| SEQ ID NO: 7 | 1.78 ± 0.10 |
| SEQ ID NO: 9 | 1.69 ± 0.29 |
| SEQ ID NO: 13 | 1.23 ± 0.11 |
| SEQ ID NO: 14 | 1.25 ± 0.02 |
| SEQ ID NO: 16 | 1.39 ± 0.26 |
| SEQ ID NO: 17 | 1.60 ± 0.46 |
| SEQ ID NO: 19 | 1.42 ± 0.15 |
| SEQ ID NO: 20 | 1.61 ± 0.21 |
| SEQ ID NO: 21 | 1.28 ± 0.07 |
| SEQ ID NO: 22 | 1.33 ± 0.07 |
| SEQ ID NO: 23 | 1.14 ± 0.24 |
| SEQ ID NO: 24 | 1.27 ± 0.16 |
| SEQ ID NO: 26 | 1.42 ± 0.11 |
| SEQ ID NO: 27 | 1.63 ± 0.03 |
| SEQ ID NO: 28 | 1.69 ± 0.03 |
| SEQ ID NO: 29 | 1.75 ± 0.09 |
| SEQ ID NO: 31 | 1.84 ± 0.06 |
| SEQ ID NO: 33 | 1.75 ± 0.21 |
| SEQ ID NO: 34 | 0.96 ± 0.05 |
| SEQ ID NO: 35 | 1.00 ± 0.08 |
| SEQ ID NO: 36 | 1.58 ± 0.05 |
| SEQ ID NO: 37 | 1.67 ± 0.02 |
| SEQ ID NO: 38 | 1.83 ± 0.03 |
| SEQ ID NO: 40 | 0.46 ± 0.06 |
| SEQ ID NO: 41 | 0.40 ± 0.01 |
| SEQ ID NO: 42 | 0.39 ± 0.08 |
| SEQ ID NO: 43 | 0.46 ± 0.10 |
| SEQ ID NO: 44 | 1.49 ± 0.39 |
| SEQ ID NO: 45 | 1.54 ± 0.35 |
| SEQ ID NO: 47 | 1.14 ± 0.23 |
| SEQ ID NO: 48 | 0.93 ± 0.08 |
| SEQ ID NO: 53 | 1.51 ± 0.37 |
| Polymyxin B | 1.30 ± 0.13 |

HBE cells were incubated with 100 μg/ml peptide or Polymyxin B for 24 hours and cell viability was tested. The data is presented as the mean ± standard deviation. As a control for 100% LDH release, Triton X-100 was added. Only peptides SEQ ID NOS: 40, 41, 42 and 43 showed any significant toxicity.

### EXAMPLE 4

### Polynucleotide Regulation by Cationic Peptides

Polynucleotide arrays were utilized to determine the effect of cationic peptides by themselves on the transcriptional response of macrophages and epithelial cells. Mouse macrophage RAW 264.7, Human Bronchial cells (HBE), or A549 human epithelial cells were plated in 150 mm tissue culture dishes at 5.6×10⁶ cells/dish, cultured overnight and then incubated with 50 μg/ml peptide or medium alone for 4 h. After stimulation, the cells were washed once with diethyl pyrocarbonate-treated PBS, and detached from the dish using a cell scraper. Total RNA was isolated using Trizol (Gibco Life Technologies). The RNA pellet was resuspended in RNase-free water containing RNase inhibitor (Ambion, Austin, Tex.). The RNA was treated with DNaseI (Clontech, Palo Alto, Calif.) for 1 h at 37° C. After adding termination mix (0.1 M EDTA [pH 8.0], 1 mg/ml glycogen), the samples were extracted once with phenol:chloroform:isoamyl alcohol (25:24:1), and once with chloroform. The RNA was then precipitated by adding 2.5 volumes of 100% ethanol and 1/10^{th} volume sodium acetate, pH 5.2. The RNA was resuspended in RNase-free water with RNase inhibitor (Ambion) and stored at −70° C. The quality of the RNA was assessed by gel electrophoresis on a 1% agarose gel. Lack of genomic DNA contamination was assessed by using the isolated RNA as a template for PCR amplification with P-actin-specific primers (5′-GTCCCTGTATGCCTCTGGTC-3′ (SEQ ID NO: 55) and 5′-GATGTCACGCACGATTTCC-3′(SEQ ID NO: 56)). Agarose gel electrophoresis and ethidium bromide staining confirmed the absence of an amplicon after 35 cycles.

Atlas cDNA Expression Arrays (Clontech, Palo Alto, Calif.), which consist of 588 selected mouse cDNAs spotted in duplicate on positively charged membranes were used for early polynucleotide array studies (Tables 18 and 19). ³²P-radiolabeled cDNA probes prepared from 5 μg total RNA were incubated with the arrays overnight at 71° C. The filters were washed extensively and then exposed to a phosphoimager screen (Molecular Dynamics, Sunnyvale, Calif.) for 3 days at 4° C. The image was captured using a Molecular Dynamics PSI phosphoimager. The hybridization signals were analyzed using Atlaslmage 1.0 Image Analysis software (Clontech) and Excel (Microsoft, Redmond, Wash.). The intensities for each spot were corrected for background levels and normalized for differences in probe labeling using the average values for 5 polynucleotides observed to vary little between the stimulation conditions: β-actin, ubiquitin, ribosomal protein S29, glyceraldehyde-3-phosphate dehydrogenase (GAPDH), and Ca²⁺ binding protein. When the normalized hybridization intensity for a given cDNA was less than 20, it was assigned a value of 20 to calculate the ratios and relative expression.

The next polynucleotide arrays used (Tables 21-26) were the Resgen Human cDNA arrays (identification number for the genome is PRHU03-S3), which consist of 7,458 human cDNAs spotted in duplicate. Probes were prepared from 15-20 μg of total RNA and labeled with Cy3 labeled dUTP. The probes were purified and hybridized to printed glass slides overnight at 42° C. and washed. After washing, the image was captured using a Virtek slide reader. The image processing software (Imagene 4.1, Marina Del Rey, Calif.) determines the spot mean intensity, median intensities, and background intensities. Normalization and analysis was performed with Genespring software (Redwood City, Calif.). Intensity values were calculated by subtracting the mean background intensity from the mean intensity value determined by Imagene. The intensities for each spot were normalized by taking the median spot intensity value from the population of spot values within a slide and comparing this value to the values of all slides in the experiment. The relative changes seen with cells treated with peptide compared to control cells can be found in the Tables below.

The other polynucleotide arrays used (Tables 27-35) were the Human Operon arrays (identification number for the genome is PRHU04-S1), which consist of about 14,000 human oligos spotted in duplicate. Probes were prepared from 10 μg of total RNA and labeled with Cy3 or Cy5 labeled dUTP. In these experiments, A549 epithelial cells were plated in 100 mm tissue culture dishes at 2.5×10⁶ cells/dish. Total RNA was isolated using RNAqueous (Ambion). DNA contamination was removed with DNA-free kit (Ambion). The probes prepared from total RNA were purified and hybridized to printed glass slides overnight at 42° C. and washed. After washing, the image was captured using a Perkin Elmer array scanner. The image processing software (Imagene 5.0, Marina Del Rey, Calif.) determines the spot mean intensity, median intensities, and background intensities. An “in house” program was used to remove background. The program calculates the bottom 10% intensity for each subgrid and subtracts this for each grid. Analysis was performed with Genespring software (Redwood City, Calif.). The intensities for each spot were normalized by taking the median spot intensity value from the population of spot values within a slide and comparing this value to the values of all slides in the experiment. The relative changes seen with cells treated with peptide compared to control cells can be found in the Tables below.

Semi-quantitative RT-PCR was performed to confirm polynucleotide array results. 1 μg RNA samples were incubated with 1 μl oligodT (500 μg/ml) and 1 μl mixed dNTP stock at 1 mM, in a 12 μl volume with DEPC treated water at 65° C. for 5 min in a thermocycler. 4 μl 5× First Strand buffer, 2 μl 0.1M DTT, and 1 μl RNaseOUT recombinant ribonuclease inhibitor (40 units/μl) were added and incubated at 42° C. for 2 min, followed by the addition of 1 μl (200 units) of Superscript II (Invitrogen, Burlington, ON). Negative controls for each RNA source were generated using parallel reactions in the absence of Superscript II. cDNAs were amplified in the presence of 5′ and 3′ primers (1.0 μM), 0.2 mM dNTP mixture, 1.5 mM MgCl, 1 U of Taq DNA polymerase (New England Biolabs, Missisauga, ON), and 1× PCR buffer. Each PCR was performed with a thermal cycler by using 30-40 cycles consisting of 30s of denaturation at 94° C., 30s of annealing at either 52° C. or 55° C. and 40s of extension at 72° C. The number of cycles of PCR was optimized to lie in the linear phase of the reaction for each primer and set of RNA samples. A housekeeping polynucleotide 0-actin was amplified in each experiment to evaluate extraction procedure and to estimate the amount of RNA. The reaction product was visualized by electrophoresis and analyzed by densitometry, with relative starting RNA concentrations calculated with reference to β-actin amplification.

Table 18 demonstrates that SEQ ID NO: 1 treatment of RAW 264.7 cells up-regulated the expression of more than 30 different polynucleotides on small Atlas microarrays with selected known polynucleotides. The polynucleotides up-regulated by peptide, SEQ ID NO: 1, were mainly from two categories: one that includes receptors (growth, chemokine, interleukin, interferon, hormone, neurotransmitter), cell surface antigens and cell adhesion and another one that includes cell-cell communication (growth factors, cytokines, chemokines, interleukin, interferons, hormones), cytoskeleton, motility, and protein turnover. The specific polynucleotides up-regulated included those encoding chemokine MCP-3, the anti-inflammatory cytokine IL-10, macrophage colony stimulating factor, and receptors such as IL-1R-2 (a putative antagonist of productive IL-1 binding to IL-1R1), PDGF receptor B, NOTCH4, LIF receptor, LFA-1, TGFβ receptor 1, G-CSF receptor, and IFNγ receptor. The peptide also up-regulated polynucleotides encoding several metalloproteinases, and inhibitors thereof, including the bone morphogenetic proteins BMP-1, BMP-2, BMP-8a, TIMP2 and TIMP3. As well, the peptide up-regulated specific transcription factors, including JunD, and-the YY and LIM-1 transcription factors, and kinases such as Etk1 and Csk demonstrating its widespread effects. It was also discovered from the polynucleotide array studies that SEQ ID NO: 1 down-regulated at least 20 polynucleotides in RAW 264.7 macrophage cells (Table 19). The polynucleotides down-regulated by peptide included DNA repair proteins and several inflammatory mediators such as MIP-1α, oncostatin M and IL-12. A number of the effects of peptide on polynucleotide expression were confirmed by RT-PCR (Table 20). The peptides, SEQ ID NO: 2, SEQ ID NO: 3, SEQ ID NO: 19, and SEQ ID NO: 1, and representative peptides from each of the formulas also altered the transcriptional responses in a human epithelial cell line using mid-sized microarrays (7835 polynucleotides). The effect of SEQ ID NO: 1 on polynucleotide expression was compared in 2 human epithelial cell lines, A549 and HBE. Polynucleotides related to the host immune response that were up-regulated by 2 peptides or more by a ratio of 2-fold more than unstimulated cells are described in Table 21. Polynucleotides that were down-regulated by 2 peptides or more by a ratio of 2-fold more than unstimulated cells are described in Table 22. In Table 23 and Table 24, the human epithelial pro-inflammatory polynucleotides that are up- and down-regulated respectively are shown. In Table 25 and Table 26 the anti-inflammatory polynucleotides affected by cationic peptides are shown. The trend becomes clear that the cationic peptides up-regulate the anti-inflammatory response and down-regulate the pro-inflammatory response. It was very difficult to find a polynucleotide related to the anti-inflammatory response that was down-regulated (Table 26). The pro-inflammatory polynucleotides upregulated by cationic peptides were mainly polynucleotides related to migration and adhesion. Of the down-regulated pro-inflammatory polynucleotides, it should be noted that all the cationic peptides affected several toll-like receptor (TLR) polynucleotides, which are very important in signaling the host response to infectious agents. An important anti-inflammatory polynucleotide that was up-regulated by all the peptides is the IL-10 receptor. IL-10 is an important cytokine involved in regulating the pro-inflammatory cytokines. These polynucleotide expression effects were also observed using primary human macrophages as observed for peptide SEQ ID NO: 6 in Tables 27 and 28. The effect of representative. peptides from each of the formulas on human epithelial cell expression of selected polynucleotides (out of 14,000 examined) is shown in Tables 31-37 below. At least 6 peptides from each formula were tested for their ability to alter human epithelial polynucleotide expression and indeed they had a wide range of stimulatory effects. In each of the formulas there were at least 50 polynucleotides commonly up-regulated by each of the peptides in the group.

TABLE 18
Polynucleotides up-regulated by peptide, SEQ ID NO: 1, treatment of RAW
macrophage cells^{a}.

| Polynucleotide/ | | Unstimulated | Ratio | Accession |
|---|---|---|---|---|
| Protein | Polynucleotide Function | Intensity | peptide:Unstimulated^{b} | Number |
| Etk1 | Tyrosine-protein kinase | 20 | 43 | M68513 |
| | receptor | | | |
| PDGFRB | Growth factor receptor | 24 | 25 | X04367 |
| | Corticotropin releasing | 20 | 23 | X72305 |
| | factor receptor | | | |
| NOTCH4 | proto-oncopolynucleotide | 48 | 18 | M80456 |
| IL-1R2 | Interleukin receptor | 20 | 16 | X59769 |
| MCP-3 | Chemokine | 56 | 14 | S71251 |
| BMP-1 | Bone | 20 | 14 | L24755 |
| | morphopolynucleotidetic | | | |
| | protein | | | |
| Endothelin | Receptor | 20 | 14 | U32329 |
| b receptor | | | | |
| c-ret | Oncopolynucleotide | 20 | 13 | X67812 |
| | precursor | | | |
| LIFR | Cytokine receptor | 20 | 12 | D26177 |
| BMP-8a | Bone | 20 | 12 | M97017 |
| | morphopolynucleotidetic | | | |
| | protein | | | |
| Zfp92 | Zinc finger protein 92 | 87 | 11 | U47104 |
| MCSF | Macrophage colony | 85 | 11 | X05010 |
| | stimulating factor 1 | | | |
| GCSFR | Granulocyte colony- | 20 | 11 | M58288 |
| | stimulating factor receptor | | | |
| IL-8RB | Chemokine receptor | 112 | 10 | D17630 |
| IL-9R | Interleukin receptor | 112 | 6 | M84746 |
| Cas | Crk-associated substrate | 31 | 6 | U48853 |
| p58/GTA | Kinase | 254 | 5 | M58633 |
| CASP2 | Caspase precursor | 129 | 5 | D28492 |
| IL-1β | Interleukin precursor | 91 | 5 | M15131 |
| precursor | | | | |
| SPI2-2 | Serine protease inhibitor | 62 | 5 | M64086 |
| C5AR | Chemokine receptor | 300 | 4 | S46665 |
| L-myc | Oncopolynucleotide | 208 | 4 | X13945 |
| IL-10 | Interleukin | 168 | 4 | M37897 |
| p19ink4 | cdk4 and cdk6 inhibitor | 147 | 4 | U19597 |
| ATOH2 | Atonal homolog 2 | 113 | 4 | U29086 |
| DNAse1 | DNase | 87 | 4 | U00478 |
| CXCR-4 | Chemokine receptor | 36 | 4 | D87747 |
| Cyclin D3 | Cyclin | 327 | 3 | U43844 |
| IL-7Rα | Interleukin receptor | 317 | 3 | M29697 |
| POLA | DNA polymerase_{α} | 241 | 3 | D17384 |
| Tie-2 | Oncopolynucleotide | 193 | 3 | S67051 |
| DNL1 | DNA ligase I | 140 | 3 | U04674 |
| BAD | Apoptosis protein | 122 | 3 | L37296 |
| GADD45 | DNA-damage-inducible | 88 | 3 | L28177 |
| | protein | | | |
| Sik | Src-related kinase | 82 | 3 | U16805 |
| integrin_{α}4 | Integrin | 2324 | 2 | X53176 |
| TGFβR1 | Growth factor receptor | 1038 | 2 | D25540 |
| LAMR1 | Receptor | 1001 | 2 | J02870 |
| Crk | Crk adaptor protein | 853 | 2 | S72408 |
| ZFX | Chromosomal protein | 679 | 2 | M32309 |
| Cyclin E1 | Cylcin | 671 | 2 | X75888 |
| POLD1 | DNA polymerase subunit | 649 | 2 | Z21848 |
| Vav | proto-oncopolynucleotide | 613 | 2 | X64361 |
| YY (NF-E1) | Transcription factor | 593 | 2 | L13968 |
| JunD | Transcription factor | 534 | 2 | J050205 |
| Csk | c-src kinase | 489 | 2 | U05247 |
| Cdk7 | Cyclin-dependent kinase | 475 | 2 | U11822 |
| MLC1A | Myosin light subunit | 453 | 2 | M19436 |
| | isoform | | | |
| ERBB-3 | Receptor | 435 | 2 | L47240 |
| UBF | Transcription factor | 405 | 2 | X60831 |
| TRAIL | Apoptosis ligand | 364 | 2 | U37522 |
| LFA-1 | Cell adhesion receptor | 340 | 2 | X14951 |
| SLAP | Src-like adaptor protein | 315 | 2 | U29056 |
| IFNGR | Interferon gamma receptor | 308 | 2 | M28233 |
| LIM-1 | Transcription factor | 295 | 2 | Z27410 |
| ATF2 | Transcription factor | 287 | 2 | S76657 |
| FST | Follistatin precursor | 275 | 2 | Z29532 |
| TIMP3 | Protease inhibitor | 259 | 2 | L19622 |
| RU49 | Transcription factor | 253 | 2 | U41671 |
| IGF-1Rα | Insulin-like growth factor | 218 | 2 | U00182 |
| | receptor | | | |
| Cyclin G2 | Cyclin | 214 | 2 | U95826 |
| fyn | Tyrosine-protein kinase | 191 | 2 | U70324 |
| BMP-2 | Bone | 186 | 2 | L25602 |
| | morphopolynucleotidetic | | | |
| | protein | | | |
| Brn-3.2 | Transcription factor | 174 | 2 | S68377 |
| POU | | | | |
| KIF1A | Kinesin family protein | 169 | 2 | D29951 |
| MRC1 | Mannose receptor | 167 | 2 | Z11974 |
| PAI2 | Protease inhibitor | 154 | 2 | X19622 |
| BKLF | CACCC Box-binding | 138 | 2 | U36340 |
| | protein | | | |
| TIMP2 | Protease inhibitor | 136 | 2 | X62622 |
| Mas | Proto-oncopolynucleotide | 131 | 2 | X67735 |
| NURR-1 | Transcription factor | 129 | 2 | S53744 |
| The cationic peptides at a concentration of 50 μg/ml were shown to potently induce the expression of several polynucleotides. Peptide was incubated with the RAW cells for 4 h and the RNA was isolated, converted into labeled cDNA probes and hybridized to Atlas arrays. The intensity of unstimulated cells is shown in the third column. The “Ratio Peptide:Unstimulated” column refers to | | | | |
| # the intensity of polynucleotide expression in peptide-simulated cells divided by the intensity of unstimulated cells. | | | | |
| The changes in the normalized intensities of the housekeeping polynucleotides ranged from 0.8-1.2 fold, validating the use of these polynucleotides for normalization. When the normalized hybridization intensity for a given cDNA was less than 20, it was assigned a value of 20 to calculate the ratios and relative expression. The array experiments were repeated 3 times with different RNA | | | | |
| # preparations and the average fold change is shown above. Polynucleotides with a two fold or greater change in relative expression levels are presented. | | | | |

TABLE 19
Polynucleotides down-regulated by SEQ ID NO: 1 treatment of RAW macrophage
cells^{a}.

| Polynucleotide/ | | Unstimulated | Ratio | Accession |
|---|---|---|---|---|
| Protein | Polynucleotide Function | Intensity | peptide:Unstimulated | Number |
| sodium channel | Voltage-gated ion channel | 257 | 0.08 | L36179 |
| XRCC1 | DNA repair protein | 227 | 0.09 | U02887 |
| ets-2 | Oncopolynucleotide | 189 | 0.11 | J04103 |
| XPAC | DNA repair protein | 485 | 0.12 | X74351 |
| EPOR | Receptor precursor | 160 | 0.13 | J04843 |
| PEA 3 | Ets-related protein | 158 | 0.13 | X63190 |
| orphan receptor | Nuclear receptor | 224 | 0.2 | U11688 |
| N-cadherin | Cell adhesion receptor | 238 | 0.23 | M31131 |
| OCT3 | Transcription factor | 583 | 0.24 | M34381 |
| PLCβ | phospholipase | 194 | 0.26 | U43144 |
| KRT18 | Intermediate filament | 318 | 0.28 | M11686 |
| | proteins | | | |
| THAM | Enzyme | 342 | 0.32 | X58384 |
| CD40L | CD40 ligand | 66 | 0.32 | X65453 |
| CD86 | T-lymphocyte antigen | 195 | 0.36 | L25606 |
| oncostatin M | Cytokine | 1127 | 0.39 | D31942 |
| PMS2 DNA | DNA repair protein | 200 | 0.4 | U28724 |
| IGFBP6 | Growth factor | 1291 | 0.41 | X81584 |
| MIP-1β | Cytokine | 327 | 0.42 | M23503 |
| ATBF1 | AT motif-binding factor | 83 | 0.43 | D26046 |
| nucleobindin | Golgi resident protein | 367 | 0.43 | M96823 |
| bcl-x | Apoptosis protein | 142 | 0.43 | L35049 |
| uromodulin | glycoprotein | 363 | 0.47 | L33406 |
| IL-12 p40 | Interleukin | 601 | 0.48 | M86671 |
| MmRad52 | DNA repair protein | 371 | 0.54 | Z32767 |
| Tob1 | Antiproliferative factor | 956 | 0.5 | D78382 |
| Ung1 | DNA repair protein | 535 | 0.51 | X99018 |
| KRT19 | Intermediate filament | 622 | 0.52 | M28698 |
| | proteins | | | |
| PLCγ | phospholipase | 251 | 0.52 | X95346 |
| Integrin α₆ | Cell adhesion receptor | 287 | 0.54 | X69902 |
| GLUT1 | Glucose transporter | 524 | 0.56 | M23384 |
| CTLA4 | immunoglobin | 468 | 0.57 | X05719 |
| | superfamily | | | |
| FRA2 | Fos-related antigen | 446 | 0.57 | X83971 |
| MTRP | Lysosome-associated | 498 | 0.58 | U34259 |
| | protein | | | |
| The cationic peptides at a concentration of 50 μg/ml were shown to reduce the expression of several polynucleotides. Peptide was incubated with the RAW cells for 4 h and the RNA was isolated, converted into labeled cDNA probes and hybridized to Atlas arrays. The intensity of unstimulated cells is shown in the third column. The “Ratio Peptide:Unstimulated” column refers to the | | | | |
| # intensity of polynucleotide expression in peptide-simulated cells divided by the intensity of unstimulated cells. The array experiments were repeated 3 times with different cells and the average fold change is shown below. Polynucleotides with an approximately two fold or greater change in relative expression levels are presented. | | | | |

TABLE 20
Polynucleotide Expression changes in response to peptide, SEQ ID
NO: 1, could be confirmed by RT-PCR.

| Polynucleotide | Array Ratio -* | RT-PCR Ratio -* |
|---|---|---|
| CXCR-4 | 4.0 ± 1.7 | 4.1 ± 0.9 |
| IL-8RB | 9.5 ± 7.6 | 7.1 ± 1.4 |
| MCP-3 | 13.5 ± 4.4 | 4.8 ± 0.88 |
| IL-10 | 4.2 ± 2.1 | 16.6 ± 6.1 |
| CD14 | 0.9 ± 0.1 | 0.8 ± 0.3 |
| MIP-1B | 0.42 ± 0.09 | 0.11 ± 0.04 |
| XRCC1 | 0.12 ± 0.01 | 0.25 ± 0.093 |
| MCP-1 | Not on array | 3.5 ± 1.4 |
| RAW 264.7 macrophage cells were incubated with 50 μg/ml of peptide or media only for 4 hours and total RNA isolated and subjected to semi-quantitative RT-PCR. Specific primer pairs for each polynucleotide were used for amplification of RNA. Amplification of β-actin was used as a positive control and for standardization. Densitometric analysis of RT-PCR products was used. The results refer | | |
| # to the relative fold change in polynucleotide expression of peptide treated cells compared to cells incubated with media alone. The data is presented as the mean ± standard error of three experiments. | | |

TABLE 21
Polynucleotides up-regulated by peptide treatment of A549 epithelial cells^{a}.

| | Unstimulated | Ratio Peptide:Unstimulated | | | | Accession |
|---|---|---|---|---|---|---|
| Polynucleotide/Protein | Intensity | ID 2 | ID 3 | ID 19 | ID 1 | Number |
| IL-1 R antagonist homolog 1 | 0.00 | 3086 | 1856 | 870 | | AI167887 |
| IL-10 R beta | 0.53 | 2.5 | 1.6 | 1.9 | 3.1 | AA486393 |
| IL-11 R alpha | 0.55 | 2.4 | 1.0 | 4.9 | 1.8 | AA454657 |
| IL-17 R | 0.54 | 2.1 | 2.0 | 1.5 | 1.9 | AW029299 |
| TNF R superfamily, member | 0.28 | 18 | 3.0 | 15 | 3.6 | AA150416 |
| 1B | | | | | | |
| TNF R superfamily, member 5 | 33.71 | 3.0 | 0.02 | | | H98636 |
| (CD40LR) | | | | | | |
| TNF R superfamily, member | 1.00 | 5.3 | 4.50 | 0.8 | | AA194983 |
| 11b | | | | | | |
| IL-8 | 0.55 | 3.6 | 17 | 1.8 | 1.1 | AA102526 |
| interleukin enhancer binding | 0.75 | 1.3 | 2.3 | 0.8 | 4.6 | AA894687 |
| factor 2 | | | | | | |
| interleukin enhancer binding | 0.41 | 2.7 | | 5.3 | 2.5 | R56553 |
| factor 1 | | | | | | |
| cytokine inducible SH2- | 0.03 | 33 | 44 | 39 | 46 | AA427521 |
| containing protein | | | | | | |
| IK cytokine, down-regulator of | 0.50 | 3.1 | 2.0 | 1.7 | 3.3 | R39227 |
| HLA II | | | | | | |
| cytokine inducible SH2- | 0.03 | 33 | 44 | 39 | 46 | AA427521 |
| containing protein | | | | | | |
| IK cytokine, down-regulator of | 0.50 | 3.1 | 2.0 | 1.7 | 3.3 | R39227 |
| HLA II | | | | | | |
| small inducible cytokine | 1.00 | 3.9 | | | 2.4 | AI922341 |
| subfamily A (Cys—Cys), | | | | | | |
| member 21 | | | | | | |
| TGFB inducible early growth | 0.90 | 2.4 | 2.1 | 0.9 | 1.1 | AI473938 |
| response 2 | | | | | | |
| NK cell R | 1.02 | 2.5 | 0.7 | 0.3 | 1.0 | AA463248 |
| CCR6 | 0.14 | 4.5 | 7.8 | 6.9 | 7.8 | N57964 |
| cell adhesion molecule | 0.25 | 4.0 | 3.9 | 3.9 | 5.1 | R40400 |
| melanoma adhesion molecule | 0.05 | 7.9 | 20 | 43 | 29.1 | AA497002 |
| CD31 | 0.59 | 2.7 | 3.1 | 1.0 | 1.7 | R22412 |
| integrin, alpha 2 (CD49B, | 1.00 | 0.9 | 2.4 | 3.6 | 0.9 | AA463257 |
| alpha 2 subunit of VLA-2 | | | | | | |
| receptor | | | | | | |
| integrin, alpha 3 (antigen | 0.94 | 0.8 | 2.5 | 1.9 | 1.1 | AA424695 |
| CD49C, alpha 3 subunit of | | | | | | |
| VLA-3 receptor) | | | | | | |
| integrin, alpha E | 0.01 | 180 | 120 | 28 | 81 | AA425451 |
| integrin, beta 1 | 0.47 | 2.1 | 2.1 | 7.0 | 2.6 | W67174 |
| integrin, beta 3 | 0.55 | 2.7 | 2.8 | 1.8 | 1.0 | AA037229 |
| integrin, beta 3 | 0.57 | 2.6 | 1.4 | 1.8 | 2.0 | AA666269 |
| integrin, beta 4 | 0.65 | 0.8 | 2.2 | 4.9 | 1.5 | AA485668 |
| integrin beta 4 binding protein | 0.20 | 1.7 | 5.0 | 6.6 | 5.3 | AI017019 |
| calcium and integrin binding | 0.21 | 2.8 | 4.7 | 9.7 | 6.7 | AA487575 |
| protein | | | | | | |
| disintegrin and | 0.46 | 3.1 | | 2.2 | 3.8 | AA279188 |
| metalloproteinase domain 8 | | | | | | |
| disintegrin and | 0.94 | 1.1 | 2.3 | 3.6 | 0.5 | H59231 |
| metalloproteinase domain 9 | | | | | | |
| disintegrin and | 0.49 | 1.5 | 2.1 | 3.3 | 2.2 | AA043347 |
| metalloproteinase domain 10 | | | | | | |
| disintegrin and | 0.44 | 1.9 | 2.3 | 2.5 | 4.6 | H11006 |
| metalloproteinase domain 23 | | | | | | |
| cadherin 1, type 1, E-cadherin | 0.42 | 8.1 | 2.2 | 2.4 | 7.3 | H97778 |
| epithelial) | | | | | | |
| cadherin 12, type 2 (N- | 0.11 | 13 | 26 | 9.5 | | AI740827 |
| cadherin 2) | | | | | | |
| protocadherin 12 | 0.09 | 14.8 | 11.5 | 2.6 | 12.4 | AI652584 |
| protocadherin gamma | 0.34 | 3.0 | 2.5 | 4.5 | 9.9 | R89615 |
| subfamily C, 3 | | | | | | |
| catenin (cadherin-associated | 0.86 | 1.2 | 2.2 | 2.4 | | AA025276 |
| protein), delta 1 | | | | | | |
| laminin R 1 (67 kD, ribosomal | 0.50 | 0.4 | 2.0 | 4.4 | 3.0 | AA629897 |
| protein SA) | | | | | | |
| killer cell lectin-like receptor | 0.11 | 9.7 | 9.0 | 4.1 | 13.4 | AA190627 |
| subfamily C, member 2 | | | | | | |
| killer cell lectin-like receptor | 1.00 | 3.2 | 1.0 | 0.9 | 1.3 | W93370 |
| subfamily C, member 3 | | | | | | |
| killer cell lectin-like receptor | 0.95 | 2.3 | 1.7 | 0.7 | 1.1 | AI433079 |
| subfamily G, member 1 | | | | | | |
| C-type lectin-like receptor-2 | 0.45 | 2.1 | 8.0 | 2.2 | 5.3 | H70491 |
| CSF 3 R | 0.40 | 1.9 | 2.5 | 3.5 | 4.0 | AA458507 |
| macrophage stimulating 1 R | 1.00 | 1.7 | 2.3 | 0.4 | 0.7 | AA173454 |
| BMP R type IA | 0.72 | 1.9 | 2.8 | 0.3 | 1.4 | W15390 |
| formyl peptide receptor 1 | 1.00 | 3.1 | 1.4 | 0.4 | | AA425767 |
| CD2 | 1.00 | 2.6 | 0.9 | 1.2 | 0.9 | AA927710 |
| CD36 | 0.18 | 8.2 | 5.5 | 6.2 | 2.5 | N39161 |
| vitamin D R | 0.78 | 2.5 | 1.3 | 1.1 | 1.4 | AA485226 |
| Human proteinase activated R-2 | 0.54 | 6.1 | 1.9 | 2.2 | | AA454652 |
| prostaglandin E receptor 3 | 0.25 | 4.1 | 4.9 | 3.8 | 4.9 | AA406362 |
| (subtype EP3) | | | | | | |
| PDGF R beta polypeptide | 1.03 | 2.5 | 1.0 | 0.5 | 0.8 | R56211 |
| VIP R 2 | 1.00 | 3.1 | | | 2.0 | AI057229 |
| growth factor receptor-bound | 0.51 | 2.2 | 2.0 | 2.4 | 0.3 | AA449831 |
| protein 2 | | | | | | |
| Mouse Mammary Turmor | 1.00 | 6.9 | | 16 | | W93891 |
| Virus Receptor homolog | | | | | | |
| adenosine A2a R | 0.41 | 3.1 | 1.8 | 4.0 | 2.5 | N57553 |
| adenosine A3 R | 0.83 | 2.0 | 2.3 | 1.0 | 1.2 | AA863086 |
| T cell R delta locus | 0.77 | 2.7 | 1.3 | | 1.8 | AA670107 |
| prostaglandin E receptor 1 | 0.65 | 7.2 | | 6.0 | 1.5 | AA972293 |
| (subtype EP1) | | | | | | |
| growth factor receptor-bound | 0.34 | | 3.0 | 6.3 | 2.9 | R24266 |
| protein 14 | | | | | | |
| Epstein-Barr virus induced | 0.61 | 1.6 | 2.4 | | 8.3 | AA037376 |
| polynucleotide 2 | | | | | | |
| complement component | 0.22 | 26 | 4.5 | 2.6 | 18.1 | AA521362 |
| receptor 2 | | | | | | |
| endothelin receptor type A | 0.07 | 12 | 14 | 14 | 16 | AA450009 |
| v-SNARE R | 0.56 | 11 | 12 | 1.8 | | AA704511 |
| tyrosine kinase, non-receptor, 1 | 0.12 | 7.8 | 8.5 | 10 | 8.7 | AI936324 |
| receptor tyrosine kinase-like | 0.40 | 7.3 | 5.0 | 1.6 | 2.5 | N94921 |
| orphan receptor 2 | | | | | | |
| protein tyrosine phosphatase, | 1.02 | 1.0 | 13.2 | 0.5 | 0.8 | AA682684 |
| non-receptor type 3 | | | | | | |
| protein tyrosine phosphatase, | 0.28 | 3.5 | 4.0 | 0.9 | 5.3 | AA434420 |
| non-receptor type 9 | | | | | | |
| protein tyrosine phosphatase, | 0.42 | 2.9 | 2.4 | 2.2 | 3.0 | AA995560 |
| non-receptor type 11 | | | | | | |
| protein tyrosine phosphatase, | 1.00 | 2.3 | 2.2 | 0.8 | 0.5 | AA446259 |
| non-receptor type 12 | | | | | | |
| protein tyrosine phosphatase, | 0.58 | 1.7 | 2.4 | 3.6 | 1.7 | AA679180 |
| non-receptor type 13 | | | | | | |
| protein tyrosine phosphatase, | 0.52 | 3.2 | 0.9 | 1.9 | 6.5 | AI668897 |
| non-receptor type 18 | | | | | | |
| protein tyrosine phosphatase, | 0.25 | 4.0 | 2.4 | 16.8 | 12.8 | H82419 |
| receptor type, A | | | | | | |
| protein tyrosine phosphatase, | 0.60 | 3.6 | 3.2 | 1.6 | 1.0 | AA045326 |
| receptor type, J | | | | | | |
| protein tyrosine phosphatase, | 0.73 | 1.2 | 2.8 | 3.0 | 1.4 | R52794 |
| receptor type, T | | | | | | |
| protein tyrosine phosphatase, | 0.20 | 6.1 | 1.2 | 5.6 | 5.0 | AA644448 |
| receptor type, U | | | | | | |
| protein tyrosine phosphatase, | 1.00 | 5.1 | | | 2.4 | AA481547 |
| receptor type, C-associated | | | | | | |
| protein | | | | | | |
| phospholipase A2 receptor 1 | 0.45 | 2.8 | 2.2 | 1.9 | 2.2 | AA086038 |
| MAP kinase-activated protein | 0.52 | 2.1 | 2.7 | 1.1 | 1.9 | W68281 |
| kinase 3 | | | | | | |
| MAP kinase kinase 6 | 0.10 | 18 | 9.6 | | 32 | H07920 |
| MAP kinase kinase 5 | 1.00 | 3.0 | 5.2 | 0.8 | 0.2 | W69649 |
| MAP kinase 7 | 0.09 | | 11.5 | 12 | 33 | H39192 |
| MAP kinase 12 | 0.49 | 2.1 | 1.7 | 2.2 | 2.0 | AI936909 |
| G protein-coupled receptor 4 | 0.40 | 3.7 | 3.0 | 2.4 | 2.5 | AI719098 |
| G protein-coupled receptor 49 | 0.05 | | 19 | 19 | 27 | AA460530 |
| G protein-coupled receptor 55 | 0.08 | 19 | 15 | 12 | | N58443 |
| G protein-coupled receptor 75 | 0.26 | 5.2 | 3.1 | 7.1 | 3.9 | H84878 |
| G protein-coupled receptor 85 | 0.20 | 6.8 | 5.4 | 4.9 | 5.0 | N62306 |
| regulator of G-protein | 0.02 | 48 | 137 | 82 | | AI264190 |
| signaling 20 | | | | | | |
| regulator of G-protein | 0.27 | | 3.7 | 8.9 | 10.6 | R39932 |
| signaling 6 | | | | | | |
| BCL2-interacting killer | 1.00 | 1.9 | | 5.2 | | AA291323 |
| (apoptosis-inducing) | | | | | | |
| apoptosis inhibitor 5 | 0.56 | 2.8 | 1.6 | 2.4 | 1.8 | AI972925 |
| caspase 6, apoptosis-related | 0.79 | 0.7 | 2.6 | 1.3 | 2.8 | W45688 |
| cysteine protease | | | | | | |
| apoptosis-related protein | 0.46 | 2.2 | 1.4 | 2.3 | 2.9 | AA521316 |
| PNAS-1 | | | | | | |
| caspase 8, apoptosis-related | 0.95 | 2.2 | 1.0 | 0.6 | 2.0 | AA448468 |
| cysteine protease | | | | | | |
| The cationic peptides at concentrations of 50 μg/ml were shown to increase the expression of several polynucleotides. Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column. The “Ratio Peptide:Unstimulated” columns refers to the intensity | | | | | | |
| # of polynucleotide expression in peptide-simulated cells divided by the intensity of unstimulated cells. | | | | | | |

TABLE 22
Polynucleotides down-regulated by peptide treatment of A549 epithelial cells^{a}.

| | Unstimulated | Ratio Peptide:Unstimulated | | | | Accession |
|---|---|---|---|---|---|---|
| Polynucleotide/Protein | Intensity | ID 2 | ID 3 | ID 19 | ID 1 | Number |
| TLR 1 | 3.22 | 0.35 | 0.31 | 0.14 | 0.19 | AI339155 |
| TLR 2 | 2.09 | 0.52 | 0.31 | 0.48 | 0.24 | T57791 |
| TLR 5 | 8.01 | 0.12 | 0.39 | | | N41021 |
| TLR 7 | 5.03 | 0.13 | 0.11 | 0.20 | 0.40 | N30597 |
| TNF receptor-associated factor 2 | 0.82 | 1.22 | 0.45 | 2.50 | 2.64 | T55353 |
| TNF receptor-associated factor 3 | 3.15 | 0.15 | | 0.72 | 0.32 | AA504259 |
| TNF receptor superfamily, member 12 | 4.17 | 0.59 | 0.24 | | 0.02 | W71984 |
| TNF R superfamily, member 17 | 2.62 | | 0.38 | 0.55 | 0.34 | AA987627 |
| TRAF and TNF receptor-associated | 1.33 | 0.75 | 0.22 | 0.67 | 0.80 | AA488650 |
| protein | | | | | | |
| IL-1 receptor, type I | 1.39 | 0.34 | 0.72 | 1.19 | 0.34 | AA464526 |
| IL-2 receptor, alpha | 2.46 | 0.41 | 0.33 | 0.58 | | AA903183 |
| IL-2 receptor, gamma (severe | 3.34 | 0.30 | 0.24 | | 0.48 | N54821 |
| combined immunodeficiency) | | | | | | |
| IL-12 receptor, beta 2 | 4.58 | 0.67 | 0.22 | | | AA977194 |
| IL-18 receptor 1 | 1.78 | 0.50 | 0.42 | 0.92 | 0.56 | AA482489 |
| TGF beta receptor III | 2.42 | 0.91 | 0.24 | 0.41 | 0.41 | H62473 |
| leukotriene b4 receptor (chemokine | 1.00 | | 1.38 | 4.13 | 0.88 | AI982606 |
| receptor-like 1) | | | | | | |
| small inducible cytokine subfamily A | 2.26 | 0.32 | | 0.44 | 1.26 | AA495985 |
| (Cys—Cys), member 18 | | | | | | |
| small inducible cytokine subfamily A | 2.22 | 0.19 | 0.38 | 0.45 | 0.90 | AI285199 |
| (Cys—Cys), member 20 | | | | | | |
| small inducible cytokine subfamily A | 2.64 | 0.38 | 0.31 | 1.53 | | AA916836 |
| (Cys—Cys), member 23 | | | | | | |
| small inducible cytokine subfamily B | 3.57 | 0.11 | 0.06 | 0.28 | 0.38 | AI889554 |
| (Cys-X-Cys), member 6 (granulocyte | | | | | | |
| chemotactic protein 2) | | | | | | |
| small inducible cytokine subfamily B | 2.02 | 0.50 | 1.07 | 0.29 | 0.40 | AA878880 |
| (Cys-X-Cys), member 10 | | | | | | |
| small inducible cytokine A3 | 2.84 | 1.79 | 0.32 | 0.35 | | AA677522 |
| (homologous to mouse Mip-1a) | | | | | | |
| cytokine-inducible kinase | 2.70 | 0.41 | 0.37 | 0.37 | 0.34 | AA489234 |
| complement component C1q receptor | 1.94 | 0.46 | 0.58 | 0.51 | 0.13 | AI761788 |
| cadherin 11, type 2, OB-cadherin | 2.00 | 0.23 | 0.57 | 0.30 | 0.50 | AA136983 |
| (osteoblast) | | | | | | |
| cadherin 3, type 1, P-cadherin | 2.11 | 0.43 | 0.53 | 0.10 | 0.47 | AA425217 |
| (placental) | | | | | | |
| cadherin, EGF LAG seven-pass. G-type | 1.67 | 0.42 | 0.41 | 1.21 | 0.60 | H39187 |
| receptor 2, flamingo (Drosophila) | | | | | | |
| homolog | | | | | | |
| cadherin 13, H-cadherin (heart) | 1.78 | 0.37 | 0.40 | 0.56 | 0.68 | R41787 |
| selectin L (lymphocyte adhesion | 4.43 | 0.03 | 0.23 | 0.61 | | H00662 |
| molecule 1) | | | | | | |
| vascular cell adhesion molecule 1 | 1.40 | 0.20 | 0.72 | 0.77 | 0.40 | H16591 |
| intercellular adhesion molecule 3 | 1.00 | 0.12 | 0.31 | 2.04 | 1.57 | AA479188 |
| integrin, alpha 1 | 2.42 | 0.41 | 0.26 | | 0.56 | AA450324 |
| integrin, alpha 7 | 2.53 | 0.57 | 0.39 | 0.22 | 0.31 | AA055979 |
| integrin, alpha 9 | 1.16 | 0.86 | 0.05 | 0.01 | 2.55 | AA865557 |
| integrin, alpha 10 | 1.00 | 0.33 | 0.18 | 1.33 | 2.25 | AA460959 |
| integrin, beta 5 | 1.00 | 0.32 | 1.52 | 1.90 | 0.06 | AA434397 |
| integrin, beta 8 | 3.27 | 0.10 | 1.14 | 0.31 | 0.24 | W56754 |
| disintegrin and metalloproteinase | 2.50 | 0.40 | 0.29 | 0.57 | 0.17 | AI205675 |
| domain 18 | | | | | | |
| disintegrin-like and metalloprotease | 2.11 | 0.32 | 0.63 | 0.47 | 0.35 | AA398492 |
| with thrombosondin type 1 motif, 3 | | | | | | |
| disintegrin-like and metalloprotease | 1.62 | 0.39 | 0.42 | 1.02 | 0.62 | AI375048 |
| with thrombospondin type 1 motif, 5 | | | | | | |
| T-cell receptor interacting molecule | 1.00 | 0.41 | 1.24 | 1.41 | 0.45 | AI453185 |
| diphtheria toxin receptor (heparin- | 1.62 | 0.49 | 0.85 | 0.62 | 0.15 | R45640 |
| binding epidermal growth factor-like | | | | | | |
| growth factor | | | | | | |
| vasoactive intestinal peptide receptor 1 | 2.31 | 0.43 | 0.31 | 0.23 | 0.54 | H73241 |
| Fc fragment of IgG, low affinity IIIb, | 3.85 | −0.20 | 0.26 | 0.76 | 0.02 | H20822 |
| receptor for (CD16) | | | | | | |
| Fc fragment of IgG, low affinity IIb, | 1.63 | 0.27 | 0.06 | 1.21 | 0.62 | R68106 |
| receptor for (CD32) | | | | | | |
| Fc fragment of IgE, high affinity I, | 1.78 | 0.43 | 0.00 | 0.56 | 0.84 | AI676097 |
| receptor for; alpha polypeptide | | | | | | |
| leukocyte immunoglobulin-like | 2.25 | 0.44 | 0.05 | 0.38 | 0.99 | N63398 |
| receptor, subfamily A | | | | | | |
| leukocyte immunoglobulin-like | 14.21 | | | 1.10 | 0.07 | AI815229 |
| receptor, subfamily B (with TM and | | | | | | |
| ITIM domains), member 3 | | | | | | |
| leukocyte immunoglobulin-like | 2.31 | 0.75 | 0.43 | 0.19 | 0.40 | AA076350 |
| receptor, subfamily B (with TM and | | | | | | |
| ITIM domains), member 4 | | | | | | |
| leukocyte immunoglobulin-like | 1.67 | 0.35 | 0.60 | 0.18 | 0.90 | H54023 |
| receptor, subfamily B | | | | | | |
| peroxisome proliferative activated | 1.18 | 0.38 | 0.85 | 0.87 | 0.26 | AI739498 |
| receptor, alpha | | | | | | |
| protein tyrosine phosphatase, receptor | 2.19 | 0.43 | | 1.06 | 0.46 | N49751 |
| type, f polypeptide (PTPRF), | | | | | | |
| interacting protein (liprin), α1 | | | | | | |
| protein tyrosine phosphatase, receptor | 1.55 | 0.44 | 0.64 | 0.30 | 0.81 | H74265 |
| type, C | | | | | | |
| protein tyrosine phosphatase, receptor | 2.08 | 0.23 | 0.37 | 0.56 | 0.48 | AA464542 |
| type, E | | | | | | |
| protein tyrosine phosphatase, receptor | 2.27 | 0.02 | 0.44 | | 0.64 | AA464590 |
| type, N polypeptide 2 | | | | | | |
| protein tyrosine phosphatase, receptor | 2.34 | 0.11 | 0.43 | 0.24 | 0.89 | AI924306 |
| type, H | | | | | | |
| protein tyrosine phosphatase, receptor- | 1.59 | 0.63 | 0.34 | 0.72 | 0.35 | AA476461 |
| type, Z polypeptide 1 | | | | | | |
| protein tyrosine phosphatase, non- | 1.07 | 0.94 | 0.43 | 0.25 | 1.13 | H03504 |
| receptor type 21 | | | | | | |
| MAP kinase 8 interacting protein 2 | 1.70 | 0.07 | 0.85 | 0.47 | 0.59 | AA418293 |
| MAP kinase kinase kinase 4 | 1.27 | 0.37 | 0.79 | 1.59 | −5.28 | AA402447 |
| MAP kinase kinase kinase 14 | 1.00 | 0.34 | 0.66 | 2.10 | 1.49 | W61116 |
| MAP kinase 8 interacting protein 2 | 2.90 | 0.16 | 0.35 | 0.24 | 0.55 | AI202738 |
| MAP kinase kinase kinase 12 | 1.48 | 0.20 | 0.91 | 0.58 | 0.68 | AA053674 |
| MAP kinase kinase kinase kinase 3 | 2.21 | 0.45 | 0.20 | 1.03 | 0.41 | AA043537 |
| MAP kinase kinase kinase 6 | 2.62 | 0.37 | 0.38 | | 0.70 | AW084649 |
| MAP kinase kinase kinase kinase 4 | 1.04 | 0.96 | 0.09 | 0.29 | 2.79 | AA417711 |
| MAP kinase kinase kinase 11 | 1.53 | 0.65 | 0.41 | 0.99 | 0.44 | R80779 |
| MAP kinase kinase kinase 10 | 1.32 | 1.23 | 0.27 | 0.50 | 0.76 | H01340 |
| MAP kinase 9 | 2.54 | 0.57 | 0.39 | 0.16 | 0.38 | AA157286 |
| MAP kinase kinase kinase 1 | 1.23 | 0.61 | 0.42 | 0.81 | 1.07 | AI538525 |
| MAP kinase kinase kinase 8 | 0.66 | 1.52 | 1.82 | 9.50 | 0.59 | W56266 |
| MAP kinase-activated protein kinase 3 | 0.52 | 2.13 | 2.68 | 1.13 | 1.93 | W68281 |
| MAP kinase kinase 2 | 0.84 | 1.20 | 3.35 | 0.02 | 1.31 | AA425826 |
| MAP kinase kinase kinase 7 | 1.00 | 0.97 | | 1.62 | 7.46 | AA460969 |
| MAP kinase 7 | 0.09 | | 11.45 | 11.80 | 33.43 | H39192 |
| MAP kinase kinase 6 | 0.10 | 17.83 | 9.61 | | 32.30 | H07920 |
| regulator of G-protein signaling 5 | 3.7397 | 0.27 | 0.06 | 0.68 | 0.18 | AA668470 |
| regulator of G-protein signaling 13 | 1.8564 | 0.54 | 0.45 | 0.07 | 1.09 | H70047 |
| G protein-coupled receptor | 1.04 | 1.84 | 0.16 | 0.09 | 0.96 | R91916 |
| G protein-coupled receptor 17 | 1.78 | 0.32 | 0.56 | 0.39 | 0.77 | AI953187 |
| G protein-coupled receptor kinase 7 | 2.62 | | 0.34 | 0.91 | 0.38 | AA488413 |
| orphan seven-transmembrane receptor, | 7.16 | 1.06 | 0.10 | 0.11 | 0.14 | AI131555 |
| chemokine related | | | | | | |
| apoptosis antagonizing transcription | 1.00 | 0.28 | 2.50 | 1.28 | 0.19 | AI439571 |
| factor | | | | | | |
| caspase 1, apoptosis-related cysteine | 2.83 | 0.44 | | 0.33 | 0.35 | T95052 |
| protease (interleukin 1, beta, | | | | | | |
| convertase) | | | | | | |
| programmed cell death 8 (apoptosis- | 1.00 | 1.07 | 0.35 | 1.94 | 0.08 | AA496348 |
| inducing factor) | | | | | | |
| The cationic peptides at concentrations of 50 μg/ml were shown to decrease the expression of several polynucleotides. Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column. The “Ratio Peptide:Unstimulated” columns refers to the intensity | | | | | | |
| # of polynucleotide expression in peptide-simulated cells divided by the intensity of unstimulated cells. | | | | | | |

TABLE 23
Pro-inflammatory polynucleotides up-regulated by peptide treatment of A549
cells.

| | Unstim. | Ratio Peptide:Unstimulated | | | | Accession |
|---|---|---|---|---|---|---|
| Polynucleotide/Protein and function | Intensity | ID 2 | ID 3 | ID 19 | ID 1 | Number |
| IL-11 Rα; Receptor for pro- | 0.55 | 2.39 | 0.98 | 4.85 | 1.82 | AA454657 |
| inflammatory cytokine, inflammation | | | | | | |
| IL-17 R; Receptor for IL-17, an inducer | 0.54 | 2.05 | 1.97 | 1.52 | 1.86 | AW029299 |
| of cytokine production in epithelial cells | | | | | | |
| small inducible cytokine subfamily A, | 1.00 | 3.88 | | | 2.41 | AI922341 |
| member 21; a chemokine | | | | | | |
| CD31; Leukocyte and cell to cell | 0.59 | 2.71 | 3.13 | 1.01 | 1.68 | R22412 |
| adhesion (PECAM) | | | | | | |
| CCR6; Receptor for chemokine MIP-3α | 0.14 | 4.51 | 7.75 | 6.92 | 7.79 | N57964 |
| integrin, alpha 2 (CD49B, alpha 2 | 1.00 | 0.89 | 2.44 | 3.62 | 0.88 | AA463257 |
| subunit of VLA-2 receptor; Adhesion to | | | | | | |
| leukocytes | | | | | | |
| integrin, alpha 3 (antigen CD49C, alpha | 0.94 | 0.79 | 2.51 | 1.88 | 1.07 | AA424695 |
| 3 subunit of VLA-3 receptor); Leukocyte | | | | | | |
| Adhesion | | | | | | |
| integrin, alpha E; Adhesion | 0.01 | 179.33 | 120.12 | 28.48 | 81.37 | AA425451 |
| integrin, beta 4; Leukocyte adhesion | 0.65 | 0.79 | 2.17 | 4.94 | 1.55 | AA485668 |
| C-type lectin-like receptor-2; Leukocyte | 0.45 | 2.09 | 7.92 | 2.24 | 5.29 | H70491 |
| adhesion | | | | | | |
| The cationic peptides at concentrations of 50 μg/ml were shown to increase the expression of certain pro-inflammatory polynucleotides (data is a subset of Table 21). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column. The “Ratio Peptide:Unstimulated” columns | | | | | | |
| # refers to the intensity of polynucleotide expression in peptide-simulated cells divided by the intensity of unstimulated cells. | | | | | | |

TABLE 24
Pro-inflammatory polynucleotides down-regulated by peptide treatment of
A549 cells.

| | Unstim | Ratio Peptide:Unstimulated | | | | Accession |
|---|---|---|---|---|---|---|
| Polynucleotide/Protein; Function | Intensity | ID 2 | ID 3 | ID 19 | ID 1 | Number |
| Toll-like receptor (TLR) 1; Response to gram | 3.22 | 0.35 | 0.31 | 0.14 | 0.19 | AI339155 |
| positive bacteria | | | | | | |
| TLR 2; Response to gram positive bacteria and | 2.09 | 0.52 | 0.31 | 0.48 | 0.24 | T57791 |
| yeast | | | | | | |
| TLR 5; May augment other TLR responses, | 8.01 | 0.12 | 0.39 | | | N41021 |
| Responsive to flagellin | | | | | | |
| TLR 7: Putative host defense mechanism | 5.03 | 0.13 | 0.11 | 0.20 | 0.40 | N30597 |
| TNF receptor-associated factor 2; Inflammation | 0.82 | 1.22 | 0.45 | 2.50 | 2.64 | T55353 |
| TNF receptor-associated factor 3; Inflammation | 3.15 | 0.15 | | 0.72 | 0.32 | AA504259 |
| TNF receptor superfamily, member 12; | 4.17 | 0.59 | 0.24 | | 0.02 | W71984 |
| Inflammation | | | | | | |
| TNF R superfamily, member 17; Inflammation | 2.62 | | 0.38 | 0.55 | 0.34 | AA987627 |
| TRAF and TNF receptor-associated protein; | 1.33 | 0.75 | 0.22 | 0.67 | 0.80 | AA488650 |
| TNF signaling | | | | | | |
| small inducible cytokine subfamily A, member | 2.26 | 0.32 | | 0.44 | 1.26 | AA495985 |
| 18; Chemokine | | | | | | |
| small inducible cytokine subfamily A, member | 2.22 | 0.19 | 0.38 | 0.45 | 0.90 | AI285199 |
| 20; Chemokine | | | | | | |
| small inducible cytokine subfamily A, member | 2.64 | 0.38 | 0.31 | 1.53 | | AA916836 |
| 23; Chemokine | | | | | | |
| small inducible cytokine subfamily B, member 6 | 3.57 | 0.11 | 0.06 | 0.28 | 0.38 | AI889554 |
| (granulocyte chemotactic protein); Chemokine | | | | | | |
| small inducible cytokine subfamily B, member | 2.02 | 0.50 | 1.07 | 0.29 | 0.40 | AA878880 |
| 10; Chemokine | | | | | | |
| small inducible cytokine A3 (homologous to | 2.84 | 1.79 | 0.32 | 0.35 | | AA677522 |
| mouse Mip-1α); Chemokine | | | | | | |
| IL-12 receptor, beta 2; Interleukin and Interferon | 4.58 | 0.67 | 0.22 | | | AA977194 |
| receptor | | | | | | |
| IL-18 receptor 1; Induces IFN-γ | 1.78 | 0.50 | 0.42 | 0.92 | 0.56 | AA482489 |
| selectin L (lymphocyte adhesion molecule 1); | 4.43 | 0.03 | 0.23 | 0.61 | | H00662 |
| Leukocyte adhesion | | | | | | |
| vascular cell adhesion molecule 1; Leukocyte | 1.40 | 0.20 | 0.72 | 0.77 | 0.40 | H16591 |
| adhesion | | | | | | |
| intercellular adhesion molecule 3; Leukocyte | 1.00 | 0.12 | 0.31 | 2.04 | 1.57 | AA479188 |
| adhesion | | | | | | |
| integrin, alpha 1; Leukocyte adhesion | 2.42 | 0.41 | 0.26 | | 0.56 | AA450324 |
| The cationic peptides at concentrations of 50 μg/ml were shown to decrease the expression of certain pro-inflammatory polynucleotides (data is a subset of Table 22). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column. The “Ratio Peptide:Unstimulated” columns | | | | | | |
| # refers to the intensity of polynucleotide expression in peptide-simulated cells divided by the intensity of unstimulated cells. | | | | | | |

TABLE 25
Anti-inflammatory polynucleotides up-regulated by peptide treatment of A549
cells.

| | Unstim | Ratio Peptide:Unstimulated | | | | Accession |
|---|---|---|---|---|---|---|
| Polynucleotide/Protein; Function | Intensity | ID 2 | ID 3 | ID 19 | ID 1 | Number |
| IL-1 R antagonist homolog 1; | 0.00 | 3085.96 | 1855.90 | 869.57 | | AI167887 |
| Inhibitor of septic shock | | | | | | |
| IL-10 R beta; Receptor for | 0.53 | 2.51 | 1.56 | 1.88 | 3.10 | AA486393 |
| cytokine synthesis inhibitor | | | | | | |
| TNF R, member 1B; Apoptosis | 0.28 | 17.09 | 3.01 | 14.93 | 3.60 | AA150416 |
| TNF R, member 5; Apoptosis | 33.71 | 2.98 | 0.02 | | | H98636 |
| (CD40L) | | | | | | |
| TNF R, member 11b; Apoptosis | 1.00 | 5.29 | 4.50 | 0.78 | | AA194983 |
| IK cytokine, down-regulator of | 0.50 | 3.11 | 2.01 | 1.74 | 3.29 | R39227 |
| HLA II; Inhibits antigen | | | | | | |
| presentation | | | | | | |
| TGFB inducible early growth | 0.90 | 2.38 | 2.08 | 0.87 | 1.11 | AI473938 |
| response 2; anti-inflammatory | | | | | | |
| cytokine | | | | | | |
| CD2; Adhesion molecule, binds | 1.00 | 2.62 | 0.87 | 1.15 | 0.88 | AA927710 |
| LFAp3 | | | | | | |
| The cationic peptides at concentrations of 50 μg/ml were shown to increase the expression of certain anti-inflammatory polynucleotides (data is a subset of Table 21). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column. The “Ratio Peptide:Unstimulated” columns | | | | | | |
| # refers to the intensity of polynucleotide expression in peptide-simulated cells divided by the intensity of unstimulated cells. | | | | | | |

TABLE 26
Anti-inflammatory polynucleotides down-regulated
by peptide treatment of A549 cells.

| Polynucleotide/ | Unstim | Ratio Peptide:Unstimulated | | | | Accession |
|---|---|---|---|---|---|---|
| Protein; Function | Intensity | ID 2 | ID 3 | ID 19 | ID 1 | Number |
| MAP kinase 9 | 2.54 | 0.57 | 0.39 | 0.16 | 0.38 | AA157286 |
| The cationic peptides at concentrations of 50 μg/ml were shown to increase the expression of certain anti-inflammatory polynucleotides (data is a subset of Table 21). Peptide was incubated with the human A549 epithelial cells for 4 h and the RNA was isolated, converted into labeled cDNA probes and hybridized to Human cDNA arrays ID#PRHU03-S3. The intensity of polynucleotides in unstimulated cells is shown in the second column. The “Ratio Peptide:Unstimulated” columns | | | | | | |
| # refers to the intensity of polynucleotide expression in peptide-simulated cells divided by the intensity of unstimulated cells. | | | | | | |

TABLE 27
Polynucleotides up-regulated by SEQ ID NO: 6, in primary human macrophages.

| | Control:Unstimulated | Ratio peptide |
|---|---|---|
| Gene (Accession Number) | cells | treated:control |
| proteoglycan 2 (Z26248) | 0.69 | 9.3 |
| Unknown (AK001843) | 26.3 | 8.2 |
| phosphorylase kinase alpha 1 (X73874) | 0.65 | 7.1 |
| actinin, alpha 3 (M86407) | 0.93 | 6.9 |
| DKFZP586B2420 protein (AL050143) | 0.84 | 5.9 |
| Unknown (AL109678) | 0.55 | 5.6 |
| transcription factor 21 (AF047419) | 0.55 | 5.4 |
| Unknown (A433612) | 0.62 | 5.0 |
| chromosome condensation 1-like (AF060219) | 0.69 | 4.8 |
| Unknown (AL137715) | 0.66 | 4.4 |
| apoptosis inhibitor 4 (U75285) | 0.55 | 4.2 |
| TERF1 (TRF1)-interacting nuclear factor 2 | 0.73 | 4.2 |
| (NM_012461) | | |
| LINE retrotransposable element 1 (M22333) | 6.21 | 4.0 |
| 1-acylglycerol-3-phosphate O-acyltransferase 1 | 0.89 | 4.0 |
| (U56417) | | |
| Vacuolar proton-ATPase, subunit D; V- | 1.74 | 4.0 |
| ATPase, subunit D (X71490) | | |
| KIAA0592 protein (AB011164) | 0.70 | 4.0 |
| potassium voltage-gated channel KQT-like | 0.59 | 3.9 |
| subfamily member 4 (AF105202) | | |
| CDC14 homolog A (AF000367) | 0.87 | 3.8 |
| histone fold proteinCHRAC17 (AF070640) | 0.63 | 3.8 |
| Cryptochrome 1 (D83702) | 0.69 | 3.8 |
| pancreatic zymogen granule membrane | 0.71 | 3.7 |
| associated protein (AB035541) | | |
| Sp3 transcription factor (X68560) | 0.67 | 3.6 |
| hypothetical protein FLJ20495 (AK000502) | 0.67 | 3.5 |
| E2F transcription factor 5, p130-binding | 0.56 | 3.5 |
| (U31556) | | |
| hypothetical protein FLJ20070 (AK000077) | 1.35 | 3.4 |
| glycoprotein IX (X52997) | 0.68 | 3.4 |
| KIAA1013 protein (AB023230) | 0.80 | 3.4 |
| eukaryotic translation initiation factor 4A, | 2.02 | 3.4 |
| isoform 2 (AL137681) | | |
| FYN-binding protein (AF198052) | 1.04 | 3.3 |
| guanine nucleotide binding protein, gamma | 0.80 | 3.3 |
| transducing activity polypeptide 1 (U41492) | | |
| glypican 1 (X54232) | 0.74 | 3.2 |
| mucosal vascular addressin cell adhesion | 0.65 | 3.2 |
| molecule 1 (U43628) | | |
| lymphocyte antigen (M38056) | 0.70 | 3.2 |
| H1 histone family, member 4 (M60748) | 0.81 | 3.0 |
| translational inhibitor protein p14.5 (X95384) | 0.78 | 3.0 |
| hypothetical protein FLJ20689 (AB032978) | 1.03 | 2.9 |
| KIAA1278 protein (AB03104) | 0.80 | 2.9 |
| unknown (AL031864) | 0.95 | 2.9 |
| chymotrypsin-like protease (X71877) | 3.39 | 2.9 |
| calumenin (NM_001219) | 2.08 | 2.9 |
| protein kinase, cAMP-dependent, regulatory, | 7.16 | 2.9 |
| type I, beta (M65066) | | |
| POU domain, class 4, transcription factor 2 | 0.79 | 2.8 |
| (U06233) | | |
| POU domain, class 2, associating factor 1 | 1.09 | 2.8 |
| (Z49194) | | |
| KIAA0532 protein (AB011104) | 0.84 | 2.8 |
| unknown (AF068289) | 1.01 | 2.8 |
| unknown (AL117643) | 0.86 | 2.7 |
| cathepsin E (M84424) | 15.33 | 2.7 |
| matrix metalloproteinase 23A (AF056200) | 0.73 | 2.7 |
| interferon receptor 2 (L42243) | 0.70 | 2.5 |
| MAP kinase kinase 1 (L11284) | 0.61 | 2.4 |
| protein kinase C, alpha (X52479) | 0.76 | 2.4 |
| c-Cbl-interacting protein (AF230904) | 0.95 | 2.4 |
| c-fos induced growth factor (Y12864) | 0.67 | 2.3 |
| cyclin-dependent kinase inhibitor 1B (S76988) | 0.89 | 2.2 |
| zinc finger protein 266 (X78924) | 1.67 | 2.2 |
| MAP kinase 14 (L35263) | 1.21 | 2.2 |
| KIAA0922 protein (AB023139) | 0.96 | 2.1 |
| bone morphogenetic protein 1 (NM_006129) | 1.10 | 2.1 |
| NADH dehydrogenase 1 alpha subcomplex, 10 | 1.47 | 2.1 |
| (AF087661) | | |
| bone morphogenetic protein receptor, type IB | 0.50 | 2.1 |
| (U89326) | | |
| interferon regulatory factor 2 (NM 002199) | 1.46 | 2.0 |
| protease, serine, 21 (AB031331) | 0.89 | 2.0 |
| The peptide SEQ ID NO: 6 at a concentration of 50 μg/ml was shown to increase the expression of many polynucleotides. Peptide was incubated with the human macrophages for 4 h and the RNA was isolated, converted into labeled cDNA probes and hybridized to Human Operon arrays (PRHU04). The intensity of polynucleotides in unstimulated cells is shown in the second column. The “Ratio peptide | | |
| # treated:Control” columns refer to the intensity of polynucleotide expression in peptide-simulated cells divided by the intensity of unstimulated cells. | | |

TABLE 28
Polynucleotides down-regulated by SEQ ID NO: 6, in primary human
macrophages.

| | Control:Unstimulated | Ratio peptide |
|---|---|---|
| Gene (Accession Number) | cells | treated:control |
| Unknown (AL049263) | 17 | 0.06 |
| integrin-linked kinase (U40282) | 2.0 | 0.13 |
| KIAA0842 protein (AB020649) | 1.1 | 0.13 |
| Unknown (AB037838) | 13 | 0.14 |
| Granulin (AF055008) | 8.6 | 0.14 |
| glutathione peroxidase 3 (NM_002084) | 1.2 | 0.15 |
| KIAA0152 gene product (D63486) | 0.9 | 0.17 |
| TGFB1-induced anti-apoptotic factor 1 (D86970) | 0.9 | 0.19 |
| disintegrin protease (Y13323) | 1.5 | 0.21 |
| proteasome subunit beta type 7 (D38048) | 0.7 | 0.22 |
| cofactor required for Sp1 transcriptional | 0.9 | 0.23 |
| activation subunit 3 (AB033042) | | |
| TNF receptor superfamily, member 14 (U81232) | 0.8 | 0.26 |
| proteasome 26S subunit non-ATPase 8 (D38047) | 1.1 | 0.28 |
| proteasome subunit beta type, 4 (D26600) | 0.7 | 0.29 |
| TNF receptor superfamily member 1B (M32315) | 1.7 | 0.29 |
| cytochrome c oxidase subunit Vic (X13238) | 3.3 | 0.30 |
| S100 calcium-binding protein A4 (M80563) | 3.8 | 0.31 |
| proteasome subunit alpha type, 6 (X59417) | 2.9 | 0.31 |
| proteasome 26S subunit non-ATPase, 10 | 1.0 | 0.32 |
| (AL031177) | | |
| MAP kinase kinase kinase 2 (NM_006609) | 0.8 | 0.32 |
| ribosomal protein L11 (X79234) | 5.5 | 0.32 |
| matrix metalloproteinase 14 (Z48481) | 1.0 | 0.32 |
| proteasome subunit beta type, 5 (D29011) | 1.5 | 0.33 |
| MAP kinase-activated protein kinase 2 (U12779) | 1.5 | 0.34 |
| caspase 3 (U13737) | 0.5 | 0.35 |
| jun D proto-oncogene (X56681) | 3.0 | 0.35 |
| proteasome 26S subunit, ATPase, 3 (M34079) | 1.3 | 0.35 |
| IL-1 receptor-like 1 (AB012701) | 0.7 | 0.35 |
| interferon alpha-inducible protein (AB019565) | 13 | 0.35 |
| SDF receptor 1 (NM_012428) | 1.6 | 0.35 |
| Cathepsin D (M63138) | 46 | 0.36 |
| MAP kinase kinase 3 (D87116) | 7.4 | 0.37 |
| TGF, beta-induced, (M77349) | 1.8 | 0.37 |
| TNF receptor superfamily, member 10b | 1.1 | 0.37 |
| (AF016266) | | |
| proteasome subunit beta type, 6 (M34079) | 1.3 | 0.38 |
| nuclear receptor binding protein (NM_013392) | 5.2 | 0.38 |
| Unknown (AL050370) | 1.3 | 0.38 |
| protease inhibitor 1 alpha-1-antitrypsin (X01683) | 0.7 | 0.40 |
| proteasome subunit alpha type, 7 (AF054185) | 5.6 | 0.40 |
| LPS-induced TNF-alpha factor (NM_004862) | 5.3 | 0.41 |
| transferrin receptor (X01060) | 14 | 0.42 |
| proteasome 26S subunit non-ATPase 13 | 1.8 | 0.44 |
| (AB009398) | | |
| MAP kinase kinase 5 (U25265) | 1.3 | 0.44 |
| Cathepsin L (X12451) | 15 | 0.44 |
| IL-1 receptor-associated kinase 1 (L76191) | 1.7 | 0.45 |
| MAP kinase kinase kinase kinase 2 (U07349) | 1.1 | 0.46 |
| peroxisome proliferative activated receptor delta | 2.2 | 0.46 |
| (AL022721) | | |
| TNF superfamily, member 15 (AF039390) | 16 | 0.46 |
| defender against cell death 1 (D15057) | 3.9 | 0.46 |
| TNF superfamily member 10 (U37518) | 287 | 0.46 |
| cathepsin H (X16832) | 14 | 0.47 |
| protease inhibitor 12 (Z81326) | 0.6 | 0.48 |
| proteasome subunit alpha type, 4 (D00763) | 2.6 | 0.49 |
| proteasome 26S subunit ATPase, 1 (L02426) | 1.8 | 0.49 |
| proteasome 26S subunit ATPase, 2 (D11094) | 2.1 | 0.49 |
| caspase 7 (U67319) | 2.4 | 0.49 |
| matrix metalloproteinase 7 (Z11887) | 2.5 | 0.49 |
| The peptide SEQ ID NO: 6 at a concentration of 50 μg/ml was shown to increase the expression of many polynucleotides. Peptide was incubated with the human macrophages for 4 h and the RNA was isolated, converted into labeled cDNA probes and hybridized to Human Operon arrays (PRHU04). The intensity of polynucleotides in unstimulated cells is shown in the second column. The “Ratio of Peptide:Control” columns refer to the intensity of | | |
| # polynucleotide expression in peptide-simulated cells divided by the intensity of unstimulated cells. | | |

TABLE 29
Polynucleotides up-regulated by SEQ ID NO: 1, in HBE cells.

| Accession | | Control:Unstimulated | Ratio peptide |
|---|---|---|---|
| Number | Gene | cells | treated:control |
| AL110161 | Unknown | 0.22 | 5218.3 |
| AF131842 | Unknown | 0.01 | 573.1 |
| AJ000730 | solute carrier family | 0.01 | 282.0 |
| Z25884 | chloride channel 1 | 0.01 | 256.2 |
| M93426 | protein tyrosine phosphatase receptor- | 0.01 | 248.7 |
| | type, zeta | | |
| X65857 | olfactory receptor, family 1, subfamily | 0.01 | 228.7 |
| | D, member 2 | | |
| M55654 | TATA box binding protein | 0.21 | 81.9 |
| AK001411 | hypothetical protein | 0.19 | 56.1 |
| D29643 | dolichyl-diphosphooligosaccharide- | 1.56 | 55.4 |
| | protein glycosyltransferase | | |
| AF006822 | myelin transcription factor 2 | 0.07 | 55.3 |
| AL117601 | Unknown | 0.05 | 53.8 |
| AL117629 | DKFZP434C245 protein | 0.38 | 45.8 |
| M59465 | tumor necrosis factor, alpha-induced | 0.50 | 45.1 |
| | protein 3 | | |
| AB013456 | aquaporin 8 | 0.06 | 41.3 |
| AJ131244 | SEC24 related gene family, member A | 0.56 | 25.1 |
| AL110179 | Unknown | 0.87 | 24.8 |
| AB037844 | Unknwon | 1.47 | 20.6 |
| Z47727 | polymerase II polypeptide K | 0.11 | 20.5 |
| AL035694 | Unknown | 0.81 | 20.4 |
| X68994 | H. sapiens CREB gene | 0.13 | 19.3 |
| AJ238379 | hypothetical protein | 1.39 | 18.5 |
| NM_003519 | H2B histone family member | 0.13 | 18.3 |
| U16126 | glutamate receptor, ionotropic kainate 2 | 0.13 | 17.9 |
| U29926 | adenosine monophosphate deaminase | 0.16 | 16.3 |
| AK001160 | hypothetical protein | 0.39 | 14.4 |
| U18018 | ets variant gene 4 | 0.21 | 12.9 |
| D80006 | KIAA0184 protein | 0.21 | 12.6 |
| AK000768 | hypothetical protein | 0.30 | 12.3 |
| X99894 | insulin promoter factor 1, | 0.26 | 12.0 |
| AL031177 | Unknown | 1.09 | 11.2 |
| AF052091 | unknown | 0.28 | 10.9 |
| L38928 | 5,10-methenyltetrahydrofolate | 0.22 | 10.6 |
| | synthetase | | |
| AL117421 | unknown | 0.89 | 10.1 |
| AL133606 | hypothetical protein | 0.89 | 9.8 |
| NM_016227 | membrane protein CH1 | 0.28 | 9.6 |
| NM_006594 | adaptor-related protein complex 4 | 0.39 | 9.3 |
| U54996 | ZW10 homolog, protein | 0.59 | 9.3 |
| AJ007557 | potassium channel, | 0.28 | 9.0 |
| AF043938 | muscle RAS oncogene | 1.24 | 8.8 |
| AK001607 | unknown | 2.74 | 8.7 |
| AL031320 | peroxisomal biogenesis factor 3 | 0.31 | 8.4 |
| D38024 | unknown | 0.31 | 8.3 |
| AF059575 | LIM homeobox TF | 2.08 | 8.2 |
| AF043724 | hepatitis A virus cellular receptor 1 | 0.39 | 8.1 |
| AK002062 | hypothetical protein | 2.03 | 8.0 |
| L13436 | natriuretic peptide receptor | 0.53 | 7.8 |
| U33749 | thyroid transcription factor 1 | 0.36 | 7.6 |
| AF011792 | cell cycle progression 2 protein | 0.31 | 7.6 |
| AK000193 | hypothetical protein | 1.18 | 6.8 |
| AF039022 | exportin, tRNA | 0.35 | 6.8 |
| M17017 | interleukin 8 | 0.50 | 6.7 |
| AF044958 | NADH dehydrogenase | 0.97 | 6.5 |
| U35246 | vacuolar protein sorting | 0.48 | 6.5 |
| AK001326 | tetraspan 3 | 1.59 | 6.5 |
| M55422 | Krueppel-related zinc finger protein | 0.34 | 6.4 |
| U44772 | palmitoyl-protein thioesterase | 1.17 | 6.3 |
| AL117485 | hypothetical protein | 0.67 | 5.9 |
| AB037776 | unknown | 0.75 | 5.7 |
| AF131827 | unknown | 0.69 | 5.6 |
| AL137560 | unknown | 0.48 | 5.2 |
| X05908 | annexin A1 | 0.81 | 5.1 |
| X68264 | melanoma adhesion molecule | 0.64 | 5.0 |
| AL161995 | neurturin | 0.86 | 4.9 |
| AF037372 | cytochrome c oxidase | 0.48 | 4.8 |
| NM_016187 | bridging integrator 2 | 0.65 | 4.8 |
| AL137758 | unknown | 0.57 | 4.8 |
| U59863 | TRAF family member-associated NFKB | 0.46 | 4.7 |
| | activator | | |
| Z30643 | chloride channel Ka | 0.70 | 4.7 |
| D16294 | acetyl-Coenzyme A acyltransferase 2 | 1.07 | 4.6 |
| AJ132592 | zinc finger protein 281 | 0.55 | 4.6 |
| X82324 | POU domain TF | 1.73 | 4.5 |
| NM_016047 | CGI-110 protein | 1.95 | 4.5 |
| AK001371 | hypothetical protein | 0.49 | 4.5 |
| M60746 | H3 histone family member D | 3.05 | 4.5 |
| AB033071 | hypothetical protein | 4.47 | 4.4 |
| AB002305 | KJAA0307 gene product | 1.37 | 4.4 |
| X92689 | UDP-N-acetyl-alpha-D- | 0.99 | 4.4 |
| | galactosamine:polypeptide N- | | |
| | acetylgalactosaminyltransferase 3 | | |
| AL049543 | glutathione peroxidase 5 | 1.62 | 4.3 |
| U43148 | patched homolog | 0.96 | 4.3 |
| M67439 | dopamine receptor D5 | 2.61 | 4.2 |
| U09850 | zinc finger protein 143 | 0.56 | 4.2 |
| L20316 | glucagon receptor | 0.75 | 4.2 |
| AB037767 | a disintegrin-like and metalloprotease | 0.69 | 4.2 |
| NM_017433 | myosin IIIA | 99.20 | 4.2 |
| D26579 | a disintegrin and metalloprotease domain 8 | 0.59 | 4.1 |
| L10333 | reticulon 1 | 1.81 | 4.1 |
| AK000761 | unknown | 1.87 | 4.1 |
| U91540 | NK homeobox family 3, A | 0.80 | 4.1 |
| Z17227 | interleukin 10 receptor, beta | 0.75 | 4.0 |
| The peptide SEQ ID NO: 1 at a concentration of 50 μg/ml was shown to increase the expression of many polynucleotides. Peptide was incubated with the human HBE epithelial cells for 4 h and the RNA was isolated, converted into labeled cDNA probes and hybridized to Human Operon arrays (PRHU04). The intensity of polynucleotides in unstimulated cells is shown in the second column. The “Ratio Peptide:Control” columns refer to the intensity of | | | |
| # polynucleotide expression in peptide-simulated cells divided by the intensity of unstimulated cells. | | | |

TABLE 30
Polynucleotides down-regulated by Peptide (50 μg/ml), SEQ ID NO: 1, in HBE cells.

| | | | Ratio of SEQ ID |
|---|---|---|---|
| Accession | | Control:Unstimulated | NO: 1- |
| Number | Gene | Cells | treated:control |
| AC004908 | Unknown | 32.4 | 0.09 |
| S70622 | G1 phase-specific gene | 43.1 | 0.10 |
| Z97056 | DEAD/H box polypeptide | 12.8 | 0.11 |
| AK002056 | hypothetical protein | 11.4 | 0.12 |
| L33930 | CD24 antigen | 28.7 | 0.13 |
| X77584 | thioredoxin | 11.7 | 0.13 |
| NM_014106 | PRO1914 protein | 25.0 | 0.14 |
| M37583 | H2A histone family member | 22.2 | 0.14 |
| U89387 | polymerase (RNA) II polypeptide D | 10.2 | 0.14 |
| D25274 | ras-related C3 botulinum toxin substrate 1 | 10.3 | 0.15 |
| J04173 | phosphoglycerate mutase 1 | 11.4 | 0.15 |
| U19765 | zinc finger protein 9 | 8.9 | 0.16 |
| X67951 | proliferation-associated gene A | 14.1 | 0.16 |
| AL096719 | profilin 2 | 20.0 | 0.16 |
| AF165217 | tropomodulin 4 | 14.6 | 0.16 |
| NM_014341 | mitochondrial carrier homolog 1 | 11.1 | 0.16 |
| AL022068 | Unknown | 73.6 | 0.17 |
| X69150 | ribosomal protein S18 | 42.8 | 0.17 |
| AL031577 | Unknown | 35.0 | 0.17 |
| AL031281 | Unknown | 8.9 | 0.17 |
| AF090094 | Human mRNA for ornithine decarboxylase | 10.3 | 0.17 |
| | antizyme, | | |
| AL022723 | HLA-G histocompatibility antigen, class I, G | 20.6 | 0.18 |
| U09813 | ATP synthase, H+ transporting mitochondrial | 9.8 | 0.18 |
| | F0 complex | | |
| AF000560 | Homo sapiens TTF-I interacting peptide 20 | 20.2 | 0.19 |
| NM_016094 | HSPC042 protein | 67.2 | 0.19 |
| AF047183 | NADH dehydrogenase | 7.5 | 0.19 |
| D14662 | anti-oxidant protein 2 (non-selenium | 8.1 | 0.19 |
| | glutathione peroxidase, acidic calcium- | | |
| | independent phospholipas | | |
| X16662 | annexin A8 | 8.5 | 0.19 |
| U14588 | paxillin | 11.3 | 0.19 |
| AL117654 | DKFZP586D0624 protein | 12.6 | 0.20 |
| AK001962 | hypothetical protein | 7.7 | 0.20 |
| L41559 | 6-pyruvoyl-tetrahydropterin | 9.1 | 0.20 |
| | synthase/dimerization cofactor of hepatocyte | | |
| | nuclear factor 1 alpha | | |
| NM_016139 | 16.7 Kd protein | 21.0 | 0.21 |
| NM_016080 | CGI-150 protein | 10.7 | 0.21 |
| U86782 | 26S proteasome-associated pad 1 homolog | 6.7 | 0.21 |
| AJ400717 | tumor protein, translationally-controlled 1 | 9.8 | 0.21 |
| X07495 | homeo box C4 | 31.0 | 0.21 |
| AL034410 | Unknown | 7.3 | 0.22 |
| X14787 | thrombospondin 1 | 26.2 | 0.22 |
| AF081192 | purine-rich element binding protein B | 6.8 | 0.22 |
| D49489 | protein disulfude isomerase-related protein | 11.0 | 0.22 |
| NM_014051 | PTD011 protein | 9.3 | 0.22 |
| AK001536 | Unknown | 98.0 | 0.22 |
| X62534 | high-mobility group protein 2 | 9.5 | 0.22 |
| AJ005259 | endothelial differentiation-related factor 1 | 6.7 | 0.22 |
| NM_000120 | epoxide hydrolase 1, microsomal | 10.0 | 0.22 |
| M38591 | S100 calcium-binding protein A10 | 23.9 | 0.23 |
| AF071596 | immediate early response 3 | 11.5 | 0.23 |
| X16396 | methylene tetrahydrofolate dehydrogenase | 8.3 | 0.23 |
| AK000934 | ATPase inhibitor precursor | 7.6 | 0.23 |
| AL117612 | Unknown | 10.7 | 0.23 |
| AF119043 | transcriptional intermediary factor 1 gamma | 7.3 | 0.23 |
| AF037066 | solute carrier family 22 member 1-like | 7.6 | 0.23 |
| | antisense | | |
| AF134406 | cytochrome c oxidase subunit | 13.3 | 0.23 |
| AE000661 | Unknown | 9.2 | 0.24 |
| AL157424 | synaptojanin 2 | 7.2 | 0.24 |
| X56468 | tyrosine 3-monooxygenase/tryptophan 5- | 7.2 | 0.24 |
| | monooxygenase activation protein, | | |
| U39318 | ubiquitin-conjugating enzyme E2D 3 | 10.7 | 0.24 |
| AL034348 | Unknown | 24.4 | 0.24 |
| D26600 | proteasome subunit beta type 4 | 11.4 | 0.24 |
| AB032987 | Unknown | 16.7 | 0.24 |
| J04182 | lysosomal-associated membrane protein 1 | 7.4 | 0.24 |
| X78925 | zinc finger protein 267 | 16.1 | 0.25 |
| NM_000805 | gastrin | 38.1 | 0.25 |
| U29700 | anti-Mullerian hormone receptor, type II | 12.0 | 0.25 |
| Z98200 | Unknown | 13.4 | 0.25 |
| U07857 | signal recognition particle | 10.3 | 0.25 |
| L05096 | Homo sapiens ribosomal protein L39 | 25.3 | 0.25 |
| AK001443 | hypothetical protein | 7.5 | 0.25 |
| K03515 | glucose phosphate isomerase | 6.2 | 0.25 |
| X57352 | interferon induced transmembrane protein 3 | 7.5 | 0.26 |
| J02883 | colipase pancreatic | 5.7 | 0.26 |
| M24069 | cold shock domain protein | 6.3 | 0.26 |
| AJ269537 | chondroitin-4-sulfotransferase | 60.5 | 0.26 |
| AL137555 | Unknown | 8.5 | 0.26 |
| U89505 | RNA binding motif protein 4 | 5.5 | 0.26 |
| U82938 | CD27-binding protein | 7.5 | 0.26 |
| X99584 | SMT3 homolog 1 | 12.8 | 0.26 |
| AK000847 | Unknown | 35.8 | 0.27 |
| NM_014463 | Lsm3 protein | 7.8 | 0.27 |
| AL133645 | Unknown | 50.8 | 0.27 |
| X78924 | zinc finger protein 266 | 13.6 | 0.27 |
| NM_004304 | anaplastic lymphoma kinase | 15.0 | 0.27 |
| X57958 | ribosomal protein L7 | 27.9 | 0.27 |
| U63542 | Unknown | 12.3 | 0.27 |
| AK000086 | hypothetical protein | 8.3 | 0.27 |
| X57138 | H2A histone family member N | 32.0 | 0.27 |
| AB023206 | KIAA0989 protein | 6.5 | 0.27 |
| AB021641 | gonadotropin inducible transcriptn repressor-1, | 5.5 | 0.28 |
| AF050639 | NADH dehydrogenase | 5.5 | 0.28 |
| M62505 | complement component 5 receptor 1 | 7.5 | 0.28 |
| X64364 | basigin | 5.8 | 0.28 |
| AJ224082 | Unknown | 22.5 | 0.28 |
| AF042165 | cytochrome c oxidase | 20.4 | 0.28 |
| AK001472 | anillin | 10.9 | 0.28 |
| X86428 | protein phosphatase 2A subunit | 12.7 | 0.28 |
| AF227132 | candidate taste receptor T2R5 | 5.1 | 0.28 |
| Z98751 | Unknown | 5.3 | 0.28 |
| D21260 | clathrin heavy polypeptide | 8.3 | 0.28 |
| AF041474 | actin-like 6 | 15.1 | 0.28 |
| NM_005258 | GTP cyclohydrolase I protein | 7.6 | 0.28 |
| L20859 | solute carrier family 20 | 9.6 | 0.29 |
| Z80783 | H2B histone family member | 9.0 | 0.29 |
| AB011105 | laminin alpha 5 | 7.1 | 0.29 |
| AL008726 | protective protein for beta-galactosidase | 5.2 | 0.29 |
| D29012 | proteasome subunit | 12.6 | 0.29 |
| X63629 | cadherin 3 P-cadherin | 6.8 | 0.29 |
| X02419 | plasminogen activator urokinase | 12.9 | 0.29 |
| X13238 | cytochrome c oxidase | 8.0 | 0.29 |
| X59798 | cyclin D1 | 12.7 | 0.30 |
| D78151 | proteasome 26S subunit | 7.6 | 0.31 |
| AF054185 | proteasome subunit | 18.8 | 0.31 |
| J03890 | surfactant pulmonary-associated protein C | 5.5 | 0.32 |
| M34079 | proteasome 26S subunit, | 5.2 | 0.33 |
| The peptide SEQ ID NO: 1 at a concentration of 50 μg/ml was shown to decrease the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in unstimulated cells is shown in the third column. The “Ratio Peptide:Control” columns refer to the intensity of | | | |
| # polynucleotide expression in peptide-simulated cells divided by the intensity of unstimulated cells. | | | |

TABLE 31
Up-regulation of Polynucleotide expression in A549 cells induced by Formula A
Peptides.

| Accession | | ctrl- | ctrl I- | | | | | | |
|---|---|---|---|---|---|---|---|---|---|
| Number | Gene | Cy3 | Cy5 | ID 5:ctrl | ID 6:ctrl | ID 7:ctrl | ID 8:ctrl | ID 9:ctrl | ID 10:ctrl |
| U12472 | glutathione S- | 0.09 | 0.31 | 13.0 | 3.5 | 4.5 | 7.0 | 4.3 | 16.4 |
| | transferase | | | | | | | | |
| X66403 | cholinergic | 0.17 | 0.19 | 7.8 | 9.9 | 6.0 | 6.4 | 5.0 | 15.7 |
| | receptor | | | | | | | | |
| AK001932 | unknown | 0.11 | 0.25 | 19.4 | 4.6 | 9.9 | 7.6 | 8.1 | 14.5 |
| X58079 | S100 calcium- | 0.14 | 0.24 | 12.2 | 7.6 | 8.1 | 4.3 | 4.5 | 13.2 |
| | binding | | | | | | | | |
| | protein | | | | | | | | |
| U18244 | solute carrier | 0.19 | 0.20 | 6.1 | 9.7 | 11.9 | 5.0 | 3.7 | 10.6 |
| | family 1 | | | | | | | | |
| U20648 | zinc finger | 0.16 | 0.13 | 5.3 | 6.2 | 5.6 | 3.1 | 6.8 | 9.5 |
| | protein | | | | | | | | |
| AB037832 | unknown | 0.10 | 0.29 | 9.0 | 4.2 | 9.4 | 3.1 | 2.6 | 8.7 |
| AC002542 | unknown | 0.15 | 0.07 | 10.5 | 15.7 | 7.8 | 10.1 | 11.7 | 8.2 |
| M89796 | membrane- | 0.15 | 0.14 | 2.6 | 6.1 | 7.6 | 3.5 | 13.3 | 8.1 |
| | spanning 4- | | | | | | | | |
| | domains, | | | | | | | | |
| | subfamily A | | | | | | | | |
| AF042163 | cytochrome c | 0.09 | 0.19 | 3.9 | 3.2 | 7.6 | 6.3 | 4.9 | 7.9 |
| | oxidase | | | | | | | | |
| AL032821 | Vanin 2 | 0.41 | 0.23 | 2.5 | 5.2 | 3.2 | 2.1 | 4.0 | 7.9 |
| U25341 | melatonin | 0.04 | 0.24 | 33.1 | 5.1 | 23.3 | 6.6 | 4.1 | 7.6 |
| | receptor 1B | | | | | | | | |
| U52219 | G protein- | 0.28 | 0.20 | 2.1 | 6.2 | 6.9 | 2.4 | 3.9 | 7.1 |
| | coupled | | | | | | | | |
| | receptor | | | | | | | | |
| X04506 | apolipoprotein B | 0.29 | 0.32 | 7.9 | 3.4 | 3.3 | 4.8 | 2.6 | 7.0 |
| AB011138 | ATPase type | 0.12 | 0.07 | 3.5 | 12.9 | 6.6 | 6.4 | 21.3 | 6.9 |
| | IV | | | | | | | | |
| AF055018 | unknown | 0.28 | 0.22 | 3.8 | 6.9 | 5.0 | 2.3 | 3.1 | 6.8 |
| AK002037 | hypothetical | 0.08 | 0.08 | 2.9 | 7.9 | 14.1 | 7.9 | 20.1 | 6.5 |
| | protein | | | | | | | | |
| AK001024 | guanine | 0.16 | 0.11 | 7.7 | 11.9 | 5.0 | 10.3 | 6.0 | 6.3 |
| | nucleotide- | | | | | | | | |
| | binding | | | | | | | | |
| | protein | | | | | | | | |
| AF240467 | TLR-7 | 0.11 | 0.10 | 20.4 | 9.0 | 3.4 | 9.4 | 12.9 | 6.1 |
| AF105367 | glucagon-like | 0.15 | 0.35 | 23.2 | 2.6 | 3.0 | 10.6 | 2.9 | 5.7 |
| | peptide 2 | | | | | | | | |
| | receptor | | | | | | | | |
| AL009183 | TNFR | 0.46 | 0.19 | 10.6 | 4.7 | 3.7 | 2.8 | 6.5 | 5.7 |
| | superfamily, | | | | | | | | |
| | member 9 | | | | | | | | |
| X54380 | pregnancy- | 0.23 | 0.08 | 4.7 | 11.9 | 7.2 | 12.7 | 3.8 | 5.5 |
| | zone protein | | | | | | | | |
| AL137736 | unknown | 0.22 | 0.15 | 2.1 | 7.2 | 3.3 | 7.1 | 4.6 | 5.5 |
| X05615 | thyroglobulin | 0.28 | 0.42 | 6.3 | 2.7 | 7.7 | 2.4 | 3.1 | 5.4 |
| D28114 | myelin- | 0.24 | 0.08 | 2.5 | 15.9 | 13.0 | 7.1 | 13.7 | 5.4 |
| | associated | | | | | | | | |
| | protein | | | | | | | | |
| AK000358 | microfibrillar- | 0.28 | 0.28 | 8.7 | 4.2 | 7.2 | 3.2 | 2.4 | 5.3 |
| | associated | | | | | | | | |
| | protein 3 | | | | | | | | |
| AK001351 | unknown | 0.12 | 0.22 | 3.9 | 7.6 | 8.7 | 3.9 | 2.3 | 5.2 |
| U79289 | unknown | 0.14 | 0.27 | 2.5 | 2.7 | 2.8 | 2.0 | 4.3 | 5.1 |
| AB014546 | ring finger | 0.12 | 0.34 | 6.8 | 2.4 | 4.1 | 2.7 | 2.0 | 5.0 |
| | protein | | | | | | | | |
| AL117428 | DKFZP434A2 | 0.10 | 0.07 | 2.8 | 16.1 | 12.8 | 9.7 | 14.2 | 4.9 |
| | 36 protein | | | | | | | | |
| AL050378 | unknown | 0.41 | 0.14 | 3.5 | 8.7 | 11.7 | 3.5 | 7.0 | 4.9 |
| AJ250562 | transmembrane | 0.13 | 0.10 | 5.2 | 5.7 | 14.2 | 3.8 | 10.3 | 4.8 |
| | 4 superfamily | | | | | | | | |
| | member 2 | | | | | | | | |
| NM_001756 | corticosteroid | 0.28 | 0.13 | 4.0 | 7.9 | 6.5 | 14.9 | 5.6 | 4.8 |
| | binding | | | | | | | | |
| | globulin | | | | | | | | |
| AL137471 | hypothetical | 0.29 | 0.05 | 3.7 | 18.0 | 6.2 | 7.2 | 16.3 | 4.7 |
| | protein | | | | | | | | |
| M19684 | protease | 0.41 | 0.14 | 3.5 | 4.6 | 5.4 | 2.8 | 9.4 | 4.7 |
| | inhibitor 1 | | | | | | | | |
| NM_001963 | epidermal | 0.57 | 0.05 | 3.4 | 6.2 | 1.8 | 32.9 | 14.7 | 4.4 |
| | growth factor | | | | | | | | |
| NM_000910 | neuropeptide | 0.62 | 0.36 | 3.1 | 2.7 | 2.3 | 2.6 | 3.1 | 4.4 |
| | Y receptor | | | | | | | | |
| AF022212 | Rho GTPase | 0.19 | 0.02 | 9.0 | 45.7 | 25.6 | 12.4 | 72.2 | 4.4 |
| | activating | | | | | | | | |
| | protein 6 | | | | | | | | |
| AK001674 | cofactor | 0.11 | 0.13 | 8.4 | 6.5 | 7.9 | 4.5 | 7.4 | 4.3 |
| | required for | | | | | | | | |
| | Sp1 | | | | | | | | |
| U51920 | signal | 0.23 | 0.27 | 3.4 | 3.8 | 2.1 | 4.1 | 8.8 | 4.2 |
| | recognition | | | | | | | | |
| | particle | | | | | | | | |
| AK000576 | hypothetical | 0.27 | 0.06 | 4.4 | 14.7 | 7.4 | 14.1 | 8.6 | 4.2 |
| | protein | | | | | | | | |
| AL080073 | unknown | 0.17 | 0.20 | 21.6 | 3.9 | 4.3 | 8.8 | 2.6 | 4.1 |
| U59628 | paired box | 0.34 | 0.06 | 3.4 | 14.1 | 5.4 | 7.9 | 4.9 | 4.1 |
| | gene 9 | | | | | | | | |
| U90548 | butyrophilin, | 0.41 | 0.31 | 2.3 | 4.7 | 5.5 | 6.8 | 3.4 | 4.1 |
| | subfamily 3, | | | | | | | | |
| | member A3 | | | | | | | | |
| M19673 | cystatin SA | 0.43 | 0.26 | 2.3 | 8.5 | 4.5 | 2.5 | 4.1 | 3.8 |
| AL161972 | ICAM 2 | 0.44 | 0.37 | 2.0 | 3.6 | 2.0 | 2.7 | 5.5 | 3.8 |
| X54938 | inositol 1,4,5- | 0.32 | 0.22 | 3.9 | 3.3 | 6.2 | 3.1 | 4.4 | 3.7 |
| | trisphosphate | | | | | | | | |
| | 3-kinase A | | | | | | | | |
| AB014575 | KIAA0675 | 0.04 | 0.13 | 46.2 | 4.5 | 10.2 | 8.0 | 6.2 | 3.4 |
| | gene product | | | | | | | | |
| M83664 | MHC II, DP | 0.57 | 0.29 | 2.9 | 2.1 | 2.0 | 3.1 | 6.6 | 3.4 |
| | beta 1 | | | | | | | | |
| AK000043 | hypothetical | 0.34 | 0.14 | 2.7 | 7.1 | 3.7 | 9.4 | 8.8 | 3.3 |
| | protein | | | | | | | | |
| U60666 | testis specific | 0.21 | 0.11 | 9.9 | 9.0 | 4.1 | 5.5 | 13.0 | 3.3 |
| | leucine rich | | | | | | | | |
| | repeat protein | | | | | | | | |
| AK000337 | hypothetical | 0.49 | 0.19 | 4.3 | 5.1 | 4.7 | 10.6 | 7.1 | 3.3 |
| | protein | | | | | | | | |
| AF050198 | putative | 0.34 | 0.15 | 7.0 | 6.3 | 3.6 | 5.6 | 11.9 | 3.3 |
| | mitochondrial | | | | | | | | |
| | space protein | | | | | | | | |
| AJ251029 | odorant- | 0.28 | 0.12 | 4.4 | 9.4 | 7.2 | 8.8 | 7.1 | 3.2 |
| | binding | | | | | | | | |
| | protein 2A | | | | | | | | |
| X74142 | forkhead box | 0.12 | 0.33 | 19.5 | 4.5 | 8.4 | 6.4 | 4.4 | 3.2 |
| | G1B | | | | | | | | |
| AB029033 | KIAA1110 | 0.35 | 0.24 | 3.1 | 2.2 | 5.6 | 5.2 | 3.1 | 3.1 |
| | protein | | | | | | | | |
| D85606 | cholecystokinin | 0.51 | 0.14 | 4.3 | 3.9 | 4.6 | 3.5 | 7.2 | 3.1 |
| | A receptor | | | | | | | | |
| X84195 | acylphosphatase | 0.32 | 0.19 | 4.8 | 3.7 | 5.0 | 11.2 | 9.8 | 3.0 |
| | 2 muscle type | | | | | | | | |
| U57971 | ATPase Ca++ | 0.29 | 0.13 | 2.2 | 7.9 | 1.8 | 6.3 | 4.8 | 3.0 |
| | transporting | | | | | | | | |
| | plasma | | | | | | | | |
| | membrane 3 | | | | | | | | |
| J02611 | apolipoprotein D | 0.28 | 0.10 | 2.8 | 11.0 | 3.7 | 10.3 | 8.4 | 3.0 |
| AF071510 | lecithin retinol | 0.07 | 0.05 | 7.9 | 3.8 | 11.7 | 46.0 | 16.3 | 3.0 |
| | acyltransferase | | | | | | | | |
| AF131757 | unknown | 0.10 | 0.08 | 4.8 | 9.0 | 44.3 | 9.3 | 10.7 | 3.0 |
| L10717 | IL2-inducible | 0.45 | 0.21 | 2.5 | 4.9 | 2.8 | 10.9 | 4.5 | 2.9 |
| | T-cell kinase | | | | | | | | |
| L32961 | 4-aminobutyrate | 0.64 | 0.32 | 3.6 | 2.9 | 3.2 | 5.3 | 2.3 | 2.9 |
| | aminotransferase | | | | | | | | |
| NM_003631 | poly (ADP- | 0.46 | 0.41 | 9.7 | 3.9 | 4.1 | 3.8 | 2.8 | 2.7 |
| | ribose) | | | | | | | | |
| | glycohydrolase | | | | | | | | |
| AF098484 | pronapsin A | 0.28 | 0.14 | 3.7 | 3.7 | 5.6 | 11.6 | 3.7 | 2.5 |
| NM_009589 | arylsulfatase D | 0.73 | 0.16 | 3.2 | 5.6 | 6.0 | 48.6 | 7.2 | 2.4 |
| M14764 | TNFR | 0.49 | 0.15 | 2.3 | 3.5 | 10.6 | 13.6 | 6.8 | 2.2 |
| | superfamily, | | | | | | | | |
| | member 16 | | | | | | | | |
| AL035250 | endothelin 3 | 0.52 | 0.14 | 2.1 | 7.3 | 4.8 | 4.5 | 3.7 | 2.2 |
| M97925 | defensin, | 0.33 | 0.07 | 4.0 | 14.7 | 7.8 | 9.4 | 3.5 | 2.1 |
| | alpha 5, | | | | | | | | |
| | Paneth cell- | | | | | | | | |
| | specific | | | | | | | | |
| D43945 | transcription | 0.46 | 0.19 | 6.6 | 2.9 | 8.2 | 4.0 | 3.5 | 2.1 |
| | factor EC | | | | | | | | |
| D16583 | histidine | 0.46 | 0.09 | 3.2 | 13.8 | 4.2 | 8.8 | 13.7 | 2.1 |
| | decarboxylase | | | | | | | | |
| The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of cDNA with the dyes Cy3 and Cy5 respectively. | | | | | | | | | |
| # The “ID#: Control” columns refer to the intensity of polynucleotide expression in peptide-simulated cells divided by the intensity of unstimulated cells. | | | | | | | | | |

TABLE 32
Up-regulation of Polynucleotide expression in A549 cells induced by Formula B
Peptides.

| Accession | | ctrl- | ctrl- | | | | | | |
|---|---|---|---|---|---|---|---|---|---|
| Number | Gene | Cy3 | Cy5 | ID 12:ctrl | ID 13:ctrl | ID 14:ctrl | ID 15:ctrl | ID 16:ctrl | ID 17:ctrl |
| AL157466 | unknown | 0.05 | 0.06 | 18.0 | 21.4 | 16.7 | 5.2 | 6.8 | 8.6 |
| AB023215 | KIAA0998 | 0.19 | 0.07 | 14.8 | 10.6 | 7.9 | 14.4 | 6.6 | 16.1 |
| | protein | | | | | | | | |
| AL031121 | unknown | 0.24 | 0.09 | 14.1 | 5.7 | 3.8 | 5.5 | 2.8 | 4.6 |
| NM_016331 | zinc finger | 0.16 | 0.08 | 12.8 | 7.2 | 11.0 | 5.3 | 11.2 | 9.7 |
| | protein | | | | | | | | |
| M14565 | cytochrome | 0.16 | 0.12 | 10.6 | 12.5 | 5.0 | 3.6 | 10.1 | 6.3 |
| | P450 | | | | | | | | |
| U22492 | G protein- | 0.28 | 0.07 | 10.4 | 8.9 | 4.8 | 10.8 | 6.6 | 3.6 |
| | coupled | | | | | | | | |
| | receptor 8 | | | | | | | | |
| U76010 | solute carrier | 0.14 | 0.07 | 9.7 | 18.6 | 3.7 | 4.8 | 5.6 | 8.9 |
| | family 30 | | | | | | | | |
| AK000685 | unknown | 0.51 | 0.10 | 9.0 | 3.1 | 2.8 | 3.9 | 15.3 | 3.0 |
| AF013620 | Immunoglobulin | 0.19 | 0.18 | 8.5 | 2.6 | 6.2 | 5.7 | 8.2 | 3.8 |
| | heavy variable | | | | | | | | |
| | 4-4 | | | | | | | | |
| AL049296 | unknown | 0.61 | 0.89 | 8.1 | 3.2 | 2.7 | 3.2 | 2.7 | 2.0 |
| AB006622 | KIAA0284 | 0.47 | 0.28 | 7.5 | 5.0 | 2.8 | 11.1 | 5.5 | 4.6 |
| | protein | | | | | | | | |
| X04391 | CD5 antigen | 0.22 | 0.13 | 7.2 | 16.7 | 2.7 | 7.7 | 6.1 | 5.9 |
| AK000067 | hypothetical | 0.80 | 0.35 | 7.1 | 4.6 | 2.1 | 3.2 | 8.5 | 2.2 |
| | protein | | | | | | | | |
| AF053712 | TNF | 0.17 | 0.08 | 6.9 | 17.7 | 3.0 | 6.2 | 12.3 | 5.2 |
| | superfamily_member | | | | | | | | |
| | 11 | | | | | | | | |
| X58079 | S100 calcium- | 0.14 | 0.24 | 6.7 | 6.7 | 5.9 | 6.5 | 5.3 | 2.5 |
| | binding protein | | | | | | | | |
| | A1 | | | | | | | | |
| M91036 | hemoglobin_gamma A | 0.48 | 0.36 | 6.7 | 14.2 | 2.1 | 2.9 | 2.7 | 4.8 |
| AF055018 | unknown | 0.28 | 0.22 | 6.3 | 10.7 | 2.7 | 2.6 | 4.6 | 6.5 |
| L17325 | pre-T/NK cell | 0.19 | 0.29 | 6.1 | 4.4 | 6.5 | 4.7 | 4.0 | 4.0 |
| | associated | | | | | | | | |
| | protein | | | | | | | | |
| D45399 | phosphodiesterase | 0.21 | 0.18 | 6.1 | 4.6 | 5.0 | 2.8 | 10.8 | 4.0 |
| AB023188 | KIAA0971 | 0.29 | 0.13 | 5.9 | 10.6 | 3.6 | 3.4 | 10.6 | 7.2 |
| | protein | | | | | | | | |
| NM_012177 | F-box protein | 0.26 | 0.31 | 5.9 | 5.5 | 3.8 | 2.8 | 3.0 | 6.8 |
| D38550 | E2F TF 3 | 0.43 | 0.39 | 5.8 | 3.4 | 2.1 | 4.5 | 2.5 | 2.4 |
| AL050219 | unknown | 0.26 | 0.04 | 5.7 | 17.0 | 3.1 | 9.2 | 30.3 | 16.1 |
| AL137540 | unknown | 0.67 | 0.79 | 5.5 | 3.2 | 3.9 | 10.9 | 2.9 | 2.3 |
| D50926 | KIAA0136 | 0.57 | 0.21 | 5.4 | 5.6 | 2.0 | 3.3 | 4.4 | 3.2 |
| | protein | | | | | | | | |
| AL137658 | unknown | 0.31 | 0.07 | 5.4 | 12.1 | 2.6 | 10.8 | 3.9 | 8.6 |
| U21931 | fructose- | 0.48 | 0.14 | 5.4 | 4.1 | 2.9 | 3.6 | 6.0 | 3.2 |
| | bisphosphatase 1 | | | | | | | | |
| AK001230 | DKFZP586D21 | 0.43 | 0.26 | 5.0 | 4.6 | 2.1 | 2.2 | 2.5 | 2.7 |
| | 1 protein | | | | | | | | |
| AL137728 | unknown | 0.67 | 0.47 | 5.0 | 5.9 | 2.2 | 6.8 | 5.9 | 2.1 |
| AB022847 | unknown | 0.39 | 0.24 | 4.5 | 2.2 | 3.5 | 4.3 | 3.8 | 3.7 |
| X75311 | mevalonate | 0.67 | 0.22 | 4.3 | 4.0 | 2.0 | 8.3 | 4.0 | 5.1 |
| | kinase | | | | | | | | |
| AK000946 | DKFZP566C24 | 0.36 | 0.29 | 4.1 | 3.8 | 3.9 | 5.4 | 25.8 | 2.7 |
| | 3 protein | | | | | | | | |
| AB023197 | KIAA0980 | 0.25 | 0.30 | 4.0 | 8.3 | 2.1 | 8.8 | 2.2 | 4.9 |
| | protein | | | | | | | | |
| AB014615 | fibroblast | 0.19 | 0.07 | 3.9 | 3.3 | 7.0 | 3.4 | 2.2 | 7.7 |
| | growth factor 8 | | | | | | | | |
| X04014 | unknown | 0.29 | 0.16 | 3.8 | 2.5 | 2.2 | 3.0 | 5.5 | 3.1 |
| U76368 | solute carrier | 0.46 | 0.17 | 3.8 | 3.8 | 2.8 | 3.2 | 4.2 | 3.0 |
| | family 7 | | | | | | | | |
| AB032436 | unknown | 0.14 | 0.21 | 3.8 | 2.7 | 6.1 | 3.2 | 4.5 | 2.6 |
| AB020683 | KIAA0876 | 0.37 | 0.21 | 3.7 | 4.2 | 2.2 | 5.3 | 2.9 | 9.4 |
| | protein | | | | | | | | |
| NM_012126 | carbohydrate | 0.31 | 0.20 | 3.7 | 5.2 | 3.2 | 3.4 | 3.9 | 2.5 |
| | sulfotransferase 5 | | | | | | | | |
| AK002037 | hypothetical | 0.08 | 0.08 | 3.7 | 17.1 | 4.6 | 12.3 | 11.0 | 8.7 |
| | protein | | | | | | | | |
| X78712 | glycerol kinase | 0.17 | 0.19 | 3.6 | 2.5 | 4.5 | 5.3 | 2.2 | 3.3 |
| | pseudogene 2 | | | | | | | | |
| NM_014178 | HSPC156 | 0.23 | 0.12 | 3.5 | 8.4 | 2.9 | 6.9 | 14.4 | 5.5 |
| | protein | | | | | | | | |
| AC004079 | homeo box A2 | 0.31 | 0.11 | 3.5 | 7.0 | 2.1 | 2.0 | 7.3 | 9.1 |
| AL080182 | unknown | 0.51 | 0.21 | 3.4 | 3.5 | 2.2 | 2.1 | 2.9 | 2.4 |
| M91036 | hemoglobin | 0.22 | 0.02 | 3.4 | 26.3 | 5.8 | 6.8 | 30.4 | 21.6 |
| | gamma G | | | | | | | | |
| AJ000512 | serum/glucocorticoid | 0.27 | 0.43 | 3.3 | 2.1 | 4.9 | 2.3 | 3.9 | 2.7 |
| | regulated | | | | | | | | |
| | kinase | | | | | | | | |
| AK002140 | hypothetical | 0.28 | 0.14 | 3.3 | 9.9 | 2.8 | 2.1 | 16.6 | 7.2 |
| | protein | | | | | | | | |
| AL137284 | unknown | 0.22 | 0.04 | 3.3 | 7.2 | 4.1 | 6.0 | 12.2 | 3.7 |
| Z11898 | POU domain_class | 0.12 | 0.29 | 3.2 | 3.7 | 8.2 | 2.5 | 6.6 | 2.2 |
| | 5 TF 1 | | | | | | | | |
| AB017016 | brain-specific | 0.27 | 0.29 | 3.1 | 2.8 | 2.5 | 2.8 | 3.3 | 5.5 |
| | protein | | | | | | | | |
| X54673 | Solute-carrier | 0.34 | 0.08 | 2.9 | 12.0 | 2.2 | 10.4 | 7.4 | 5.9 |
| | family 6 | | | | | | | | |
| AL033377 | unknown | 0.40 | 0.22 | 2.6 | 2.6 | 2.6 | 2.3 | 4.5 | 2.2 |
| X85740 | CCR4 | 0.34 | 0.05 | 2.6 | 2.3 | 2.6 | 2.5 | 12.5 | 5.2 |
| AB010419 | core-binding | 0.59 | 0.20 | 2.5 | 12.8 | 2.0 | 2.8 | 2.9 | 5.9 |
| | factor | | | | | | | | |
| AL109726 | uknown | 0.14 | 0.15 | 2.3 | 9.0 | 4.3 | 4.4 | 2.6 | 3.7 |
| NM_012450 | sulfate | 0.15 | 0.10 | 2.2 | 3.1 | 8.2 | 9.9 | 4.7 | 5.9 |
| | transporter 1 | | | | | | | | |
| J04599 | biglycan | 0.39 | 0.30 | 2.1 | 3.3 | 6.6 | 2.2 | 2.7 | 5.4 |
| AK000266 | hypothetical | 0.49 | 0.35 | 2.1 | 3.5 | 3.5 | 6.6 | 4.3 | 4.0 |
| | protein | | | | | | | | |
| The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of cDNA with the dyes Cy3 and Cy5 respectively. | | | | | | | | | |
| # The “ID#: Control” columns refer to the intensity of polynucleotide expression in peptide-simulated cells divided by the intensity of unstimulated cells. | | | | | | | | | |

TABLE 33
Up-regulation of Polynucleotide expression in A549 cells induced by Formula C
Peptides.

| Accession | | ctrl- | ctrl- | | | | | | |
|---|---|---|---|---|---|---|---|---|---|
| Number | Gene | Cy3 | Cy5 | ID 19:ctrl | ID 20:ctrl | ID 21:ctrl | ID 22:ctrl | ID 23:ctrl | ID 24:ctrl |
| NM_014139 | sodium | 0.04 | 0.05 | 31.6 | 25.2 | 18.0 | 9.7 | 22.2 | 11.2 |
| | channel | | | | | | | | |
| | voltage- | | | | | | | | |
| | gated, | | | | | | | | |
| X84003 | TATA box | 0.47 | 0.07 | 31.8 | 12.7 | 2.5 | 2.8 | 18.0 | 14.2 |
| | binding | | | | | | | | |
| | protein | | | | | | | | |
| AF144412 | lens epithelial | 0.25 | 0.07 | 23.9 | 8.0 | 6.8 | 3.4 | 16.2 | 3.5 |
| | cell protein | | | | | | | | |
| AL080107 | unknown | 0.11 | 0.06 | 17.8 | 34.4 | 12.4 | 6.2 | 5.4 | 7.9 |
| AF052116 | unknown | 0.34 | 0.07 | 15.5 | 3.9 | 9.2 | 3.0 | 6.9 | 2.7 |
| AB033063 | unknown | 0.46 | 0.13 | 15.2 | 10.3 | 4.0 | 2.6 | 7.2 | 11.2 |
| AK000258 | hypothetical | 0.27 | 0.07 | 13.9 | 8.0 | 3.5 | 3.4 | 26.5 | 11.5 |
| | protein | | | | | | | | |
| NM_006963 | zinc finger | 0.10 | 0.08 | 12.8 | 6.8 | 6.2 | 5.9 | 17.2 | 1241.2 |
| | protein | | | | | | | | |
| NM_014099 | PRO1768 | 0.30 | 0.06 | 12.3 | 17.4 | 5.4 | 5.4 | 19.5 | 3.4 |
| | protein | | | | | | | | |
| AK000996 | hypothetical | 0.17 | 0.07 | 10.0 | 8.0 | 9.7 | 7.4 | 20.7 | 16.3 |
| | protein | | | | | | | | |
| M81933 | cell division | 0.13 | 0.21 | 8.8 | 7.8 | 19.6 | 15.6 | 4.8 | 3.8 |
| | cycle 25A | | | | | | | | |
| AF181286 | unknown | 0.05 | 0.22 | 8.8 | 2.7 | 12.0 | 35.6 | 5.9 | 2.3 |
| AJ272208 | IL-1R | 0.22 | 0.17 | 8.8 | 2.9 | 5.0 | 3.2 | 9.8 | 7.3 |
| | accessory | | | | | | | | |
| | protein-like 2 | | | | | | | | |
| AF030555 | fatty-acid- | 0.10 | 0.39 | 8.7 | 2.2 | 11.3 | 9.9 | 3.0 | 2.1 |
| | Coenzyme A | | | | | | | | |
| | ligase | | | | | | | | |
| AL050125 | unknown | 0.23 | 0.07 | 8.6 | 14.3 | 5.2 | 2.8 | 18.7 | 8.3 |
| AB011096 | KIAA0524 | 0.21 | 0.08 | 8.5 | 24.4 | 4.7 | 6.8 | 10.4 | 7.5 |
| | protein | | | | | | | | |
| J03068 | N- | 0.54 | 0.21 | 8.3 | 2.4 | 2.2 | 4.1 | 3.0 | 6.0 |
| | acylaminoacyl- | | | | | | | | |
| | peptide | | | | | | | | |
| | hydrolase | | | | | | | | |
| M33906 | MHC class | 0.14 | 0.08 | 7.6 | 4.5 | 15.2 | 6.1 | 7.5 | 7.9 |
| | II, DQ alpha 1 | | | | | | | | |
| AJ272265 | secreted | 0.21 | 0.09 | 7.6 | 9.0 | 3.3 | 4.9 | 18.8 | 14.5 |
| | phosphoprotein | | | | | | | | |
| J00210 | interferon | 0.41 | 0.07 | 7.2 | 15.0 | 2.8 | 3.1 | 11.0 | 4.3 |
| | alpha 13 | | | | | | | | |
| AK001952 | hypothetical | 0.42 | 0.21 | 6.9 | 4.9 | 2.5 | 3.1 | 7.6 | 4.5 |
| | protein | | | | | | | | |
| X54131 | protein | 0.09 | 0.20 | 6.4 | 6.5 | 7.7 | 15.0 | 5.6 | 4.1 |
| | tyrosine | | | | | | | | |
| | phosphatase, | | | | | | | | |
| | receptor type, | | | | | | | | |
| AF064493 | LIM binding | 0.46 | 0.14 | 5.9 | 5.6 | 2.2 | 2.9 | 8.5 | 5.8 |
| | domain 2 | | | | | | | | |
| AL117567 | DKFZP566O | 0.44 | 0.22 | 5.8 | 3.3 | 2.9 | 2.3 | 5.7 | 14.9 |
| | 084 protein | | | | | | | | |
| L40933 | phosphogluco | 0.16 | 0.03 | 5.6 | 11.0 | 4.8 | 3.5 | 8.5 | 76.3 |
| | mutase 5 | | | | | | | | |
| M27190 | regenerating | 0.19 | 0.28 | 5.3 | 3.0 | 3.8 | 3.6 | 5.8 | 3.6 |
| | islet-derived | | | | | | | | |
| | 1 alpha | | | | | | | | |
| AL031121 | unknown | 0.24 | 0.09 | 5.3 | 3.8 | 3.2 | 3.9 | 3.0 | 27.9 |
| U27655 | regulator of | 0.24 | 0.29 | 5.0 | 9.0 | 4.5 | 8.3 | 4.2 | 4.5 |
| | G-protein | | | | | | | | |
| | signaling | | | | | | | | |
| AB037786 | unknown | 0.12 | 0.03 | 4.7 | 54.1 | 2.8 | 2.3 | 2.2 | 11.0 |
| X73113 | myosin- | 0.29 | 0.13 | 4.7 | 6.5 | 6.0 | 2.4 | 6.7 | 6.3 |
| | binding | | | | | | | | |
| | protein C | | | | | | | | |
| AB010962 | matrix | 0.08 | 0.12 | 4.7 | 6.2 | 2.4 | 4.7 | 10.9 | 4.2 |
| | metalloproteinase | | | | | | | | |
| AL096729 | unknown | 0.36 | 0.13 | 4.7 | 7.7 | 3.2 | 2.4 | 6.3 | 6.2 |
| AB018320 | Arg/Abl- | 0.16 | 0.18 | 4.6 | 7.1 | 3.0 | 3.3 | 5.8 | 8.9 |
| | interacting | | | | | | | | |
| | protein | | | | | | | | |
| AK001024 | guanine | 0.16 | 0.11 | 4.6 | 2.0 | 9.8 | 2.6 | 7.6 | 14.1 |
| | nucleotide- | | | | | | | | |
| | binding | | | | | | | | |
| | protein | | | | | | | | |
| AJ275355 | unknown | 0.15 | 0.08 | 4.6 | 17.3 | 5.4 | 9.2 | 5.1 | 5.5 |
| U21931 | fructose- | 0.48 | 0.14 | 4.6 | 4.3 | 2.6 | 2.1 | 8.4 | 9.6 |
| | bisphosphatase 1 | | | | | | | | |
| X66403 | cholinergic | 0.17 | 0.19 | 4.4 | 9.0 | 10.9 | 9.3 | 5.1 | 6.7 |
| | receptor | | | | | | | | |
| X67734 | contactin 2 | 0.25 | 0.09 | 4.3 | 6.8 | 3.1 | 5.8 | 7.9 | 8.4 |
| U92981 | unknown | 0.20 | 0.23 | 4.3 | 3.2 | 4.8 | 5.6 | 5.4 | 6.3 |
| X68879 | empty | 0.05 | 0.08 | 4.3 | 2.0 | 12.3 | 2.7 | 5.6 | 4.7 |
| | spiracles | | | | | | | | |
| | homolog 1 | | | | | | | | |
| AL137362 | unknown | 0.22 | 0.22 | 4.2 | 4.1 | 2.7 | 4.1 | 9.3 | 4.2 |
| NM_001756 | corticosteroid | 0.28 | 0.13 | 4.1 | 10.6 | 3.9 | 2.7 | 10.3 | 5.5 |
| | binding | | | | | | | | |
| | globulin | | | | | | | | |
| U80770 | unknown | 0.31 | 0.14 | 4.1 | 4.1 | 23.3 | 2.7 | 7.0 | 10.1 |
| AL109792 | unknown | 0.16 | 0.19 | 4.0 | 4.5 | 4.3 | 8.8 | 8.7 | 3.9 |
| X65962 | cytochrome | 0.33 | 0.05 | 3.8 | 25.3 | 5.7 | 5.1 | 19.8 | 12.0 |
| | P-450 | | | | | | | | |
| AK001856 | unknown | 0.40 | 0.21 | 3.8 | 7.0 | 2.6 | 3.1 | 2.9 | 7.8 |
| AL022723 | MHC, class I, F | 0.55 | 0.18 | 3.7 | 5.7 | 4.4 | 2.3 | 3.3 | 5.2 |
| D38449 | putative G | 0.18 | 0.09 | 3.5 | 11.1 | 13.3 | 5.8 | 4.8 | 5.2 |
| | protein | | | | | | | | |
| | coupled | | | | | | | | |
| | receptor | | | | | | | | |
| AL137489 | unknown | 0.74 | 0.26 | 3.3 | 2.9 | 2.6 | 3.3 | 2.5 | 5.4 |
| AB000887 | small | 0.76 | 0.18 | 3.3 | 5.0 | 2.6 | 2.4 | 5.9 | 10.3 |
| | inducible | | | | | | | | |
| | cytokine | | | | | | | | |
| | subfamily A | | | | | | | | |
| NM_012450 | sulfate | 0.15 | 0.10 | 3.3 | 9.0 | 10.0 | 10.9 | 4.6 | 8.7 |
| | transport 1 | | | | | | | | |
| U86529 | glutathione | 0.55 | 0.15 | 3.2 | 6.8 | 4.4 | 2.3 | 9.3 | 5.1 |
| | S-transferase | | | | | | | | |
| | zeta 1 | | | | | | | | |
| AK001244 | unknown | 0.79 | 0.31 | 3.2 | 5.5 | 2.3 | 2.3 | 3.9 | 2.8 |
| AL133602 | unknown | 0.16 | 0.21 | 3.1 | 7.8 | 8.7 | 2.6 | 4.1 | 5.6 |
| AB033080 | cell cycle | 0.31 | 0.31 | 3.1 | 4.6 | 3.0 | 3.5 | 2.2 | 4.2 |
| | progression 8 | | | | | | | | |
| | protein | | | | | | | | |
| AF023466 | putative | 0.27 | 0.18 | 3.1 | 5.0 | 4.2 | 7.4 | 10.1 | 3.8 |
| | glycine-N- | | | | | | | | |
| | acyltransferase | | | | | | | | |
| AL117457 | cofilin 2 | 0.68 | 0.53 | 3.0 | 4.6 | 3.3 | 2.4 | 7.4 | 3.4 |
| AC007059 | unknown | 0.37 | 0.35 | 3.0 | 5.7 | 3.1 | 2.4 | 2.6 | 2.4 |
| U60179 | growth | 0.34 | 0.21 | 2.9 | 3.5 | 2.3 | 3.1 | 8.0 | 4.7 |
| | hormone | | | | | | | | |
| | receptor | | | | | | | | |
| M37238 | phospholipase | 0.60 | 0.36 | 2.9 | 2.0 | 3.2 | 2.1 | 2.9 | 4.6 |
| | C, gamma 2 | | | | | | | | |
| L22569 | cathepsin B | 0.32 | 0.12 | 2.9 | 2.1 | 6.2 | 3.0 | 13.1 | 16.7 |
| M80359 | MAP/microtubule | 0.37 | 0.76 | 2.9 | 3.1 | 6.1 | 7.6 | 2.1 | 3.3 |
| | affinity- | | | | | | | | |
| | regulating | | | | | | | | |
| | kinase 3 | | | | | | | | |
| S70348 | Integrin beta 3 | 0.58 | 0.31 | 2.6 | 4.8 | 4.1 | 2.6 | 2.6 | 2.6 |
| L13720 | growth | 0.36 | 0.26 | 2.4 | 2.5 | 6.8 | 4.8 | 3.9 | 3.7 |
| | arrest- | | | | | | | | |
| | specific 6 | | | | | | | | |
| AL049423 | unknown | 0.33 | 0.30 | 2.4 | 3.7 | 3.8 | 2.8 | 2.9 | 3.4 |
| AL050201 | unknown | 0.68 | 0.29 | 2.2 | 3.1 | 3.7 | 3.0 | 3.0 | 2.2 |
| AF050078 | growth arrest | 0.87 | 0.33 | 2.1 | 8.4 | 2.5 | 2.2 | 2.6 | 4.4 |
| | specific 11 | | | | | | | | |
| AK001753 | hypothetical | 0.53 | 0.28 | 2.1 | 5.0 | 2.2 | 2.8 | 3.6 | 4.6 |
| | protein | | | | | | | | |
| X05323 | unknown | 0.39 | 0.13 | 2.1 | 7.8 | 2.6 | 2.4 | 21.5 | 3.5 |
| AB014548 | KIAA0648 | 0.61 | 0.30 | 2.0 | 2.4 | 4.8 | 3.4 | 4.9 | 3.9 |
| | protein | | | | | | | | |
| The peptides at a concentration of 50 μg/ml were shown to increase the expression of many polynucleotides. Peptide was incubated with the human A549 epithelial cells for 4 h and the RNA was isolated, converted into labeled cDNA probes and hybridized to Human Operon arrays (PRHU04). The intensity of polynucleotides in control, unstimulated cells are shown in the second and third columns for labeling of cDNA with the dyes Cy3 and Cy5 respectively. | | | | | | | | | |
| # The “ID#: Control” columns refer to the intensity of polynucleotide expression in peptide-simulated cells divided by the intensity of unstimulated cells. | | | | | | | | | |

TABLE 34
Up-regulation of Polynucleotide expression in A549 cells induced by Formula D
Peptides.

| Accession | | ctrl- | ctrl- | | | | | | |
|---|---|---|---|---|---|---|---|---|---|
| Number | Gene | Cy3 | Cy5 | ID 26:ctrl | ID 27:ctrl | ID 28:ctrl | ID 29:ctrl | ID 30:ctrl | ID 31:ctrl |
| U68018 | MAD homolog 2 | 0.13 | 0.71 | 11.2 | 2.2 | 8.0 | 2.3 | 6.7 | 25.6 |
| NM_016015 | CGI-68 protein | 0.92 | 1.59 | 2.3 | 2.3 | 3.5 | 3.7 | 3.4 | 22.9 |
| AF071510 | lecithin retinol | 0.07 | 0.05 | 15.4 | 10.3 | 5.3 | 44.1 | 2.1 | 21.2 |
| | acyltransferase | | | | | | | | |
| AC005154 | unkown | 0.17 | 1.13 | 2.7 | 7.2 | 12.6 | 6.4 | 3.3 | 20.6 |
| M81933 | cell division | 0.13 | 0.21 | 4.3 | 3.1 | 3.2 | 4.3 | 5.6 | 18.2 |
| | cycle 25A | | | | | | | | |
| AF124735 | LIM HOX | 0.17 | 0.21 | 2.1 | 4.4 | 5.9 | 5.2 | 7.6 | 17.0 |
| | gene 2 | | | | | | | | |
| AL110125 | unknown | 0.30 | 0.08 | 5.0 | 2.7 | 6.8 | 10.2 | 2.8 | 12.0 |
| NM_004732 | potassium | 0.15 | 0.16 | 7.6 | 4.0 | 3.4 | 2.2 | 2.9 | 11.4 |
| | voltage-gated | | | | | | | | |
| | channel | | | | | | | | |
| AF030555 | fatty-acid- | 0.10 | 0.39 | 10.5 | 2.2 | 6.4 | 3.0 | 5.1 | 10.7 |
| | Coenzyme A | | | | | | | | |
| | ligase_long- | | | | | | | | |
| | chain 4 | | | | | | | | |
| AF000237 | 1-acylglycerol- | 1.80 | 2.37 | 3.4 | 2.5 | 2.4 | 2.1 | 3.7 | 9.9 |
| | 3-phosphate O- | | | | | | | | |
| | acyltransferase 2 | | | | | | | | |
| AL031588 | hypothetical | 0.40 | 0.26 | 5.8 | 20.2 | 2.8 | 4.7 | 5.6 | 9.1 |
| | protein | | | | | | | | |
| AL080077 | unknown | 0.15 | 0.21 | 2.4 | 2.0 | 11.9 | 3.8 | 2.3 | 8.7 |
| NM_014366 | putative | 0.90 | 2.52 | 2.4 | 4.3 | 2.4 | 2.6 | 3.0 | 8.6 |
| | nucleotide | | | | | | | | |
| | binding | | | | | | | | |
| | protein_estradiol- | | | | | | | | |
| | induced | | | | | | | | |
| AB002359 | phosphoribosyl | 0.81 | 2.12 | 3.2 | 2.7 | 5.5 | 2.5 | 2.8 | 6.9 |
| | formylglycina | | | | | | | | |
| | midine | | | | | | | | |
| | synthase | | | | | | | | |
| U33547 | MHC class II | 0.14 | 0.16 | 2.5 | 5.3 | 4.5 | 5.0 | 3.1 | 6.6 |
| | antigen HLA- | | | | | | | | |
| | DRB6 mRNA_{—} | | | | | | | | |
| AL133051 | unknown | 0.09 | 0.07 | 7.7 | 6.3 | 5.4 | 23.1 | 5.4 | 6.5 |
| AK000576 | hypothetical | 0.27 | 0.06 | 7.1 | 9.3 | 5.0 | 6.9 | 2.9 | 6.2 |
| | protein | | | | | | | | |
| AF042378 | spindle pole | 0.36 | 0.39 | 3.3 | 3.0 | 9.5 | 4.5 | 3.4 | 6.2 |
| | body protein | | | | | | | | |
| AF093265 | Homer | 0.67 | 0.53 | 2.7 | 13.3 | 6.5 | 5.0 | 2.9 | 6.2 |
| | neuronal | | | | | | | | |
| | immediate | | | | | | | | |
| | early gene_3 | | | | | | | | |
| D80000 | Segregation of | 1.01 | 1.56 | 3.6 | 2.5 | 4.9 | 3.2 | 6.3 | 6.1 |
| | mitotic | | | | | | | | |
| | chromosomes 1 | | | | | | | | |
| AF035309 | proteasome | 3.61 | 4.71 | 2.7 | 6.6 | 5.2 | 4.9 | 2.7 | 6.0 |
| | 26S subunit | | | | | | | | |
| | ATPase 5 | | | | | | | | |
| M34175 | adaptor-related | 4.57 | 5.13 | 3.2 | 3.1 | 4.0 | 4.6 | 2.7 | 6.0 |
| | protein | | | | | | | | |
| | complex 2 beta | | | | | | | | |
| | 1 subunit | | | | | | | | |
| AB020659 | KIAA0852 | 0.18 | 0.37 | 4.1 | 7.6 | 5.7 | 4.8 | 2.5 | 5.7 |
| | protein | | | | | | | | |
| NM_004862 | LPS-induced | 2.61 | 3.36 | 3.8 | 4.8 | 4.1 | 4.9 | 3.2 | 5.6 |
| | TNF-alpha | | | | | | | | |
| | factor | | | | | | | | |
| U00115 | zinc finger | 0.51 | 0.07 | 18.9 | 2.2 | 3.5 | 7.2 | 21.2 | 5.6 |
| | protein 51 | | | | | | | | |
| AF088868 | fibrousheathin | 0.45 | 0.20 | 4.7 | 10.0 | 3.2 | 6.4 | 6.0 | 5.6 |
| | II | | | | | | | | |
| AK001890 | unknown | 0.42 | 0.55 | 2.4 | 3.5 | 3.6 | 2.3 | 2.2 | 5.6 |
| AL137268 | KIAA0759 | 0.49 | 0.34 | 3.8 | 2.3 | 5.0 | 3.5 | 3.3 | 5.4 |
| | protein | | | | | | | | |
| X63563 | polymerase II | 1.25 | 1.68 | 2.5 | 8.1 | 3.4 | 4.8 | 5.2 | 5.4 |
| | polypeptide B | | | | | | | | |
| D12676 | CD36 antigen | 0.35 | 0.39 | 2.9 | 3.4 | 2.6 | 2.2 | 3.5 | 5.3 |
| AK000161 | hypothetical | 1.06 | 0.55 | 3.4 | 8.7 | 2.1 | 6.7 | 2.9 | 5.1 |
| | protein | | | | | | | | |
| AF052138 | unknown | 0.64 | 0.51 | 2.9 | 2.8 | 2.7 | 5.2 | 3.6 | 5.0 |
| AL096803 | unknown | 0.36 | 0.03 | 20.1 | 18.3 | 3.7 | 19.3 | 16.1 | 4.9 |
| S49953 | DNA-binding | 0.70 | 0.15 | 3.7 | 4.0 | 2.1 | 6.6 | 4.0 | 4.8 |
| | transcriptional | | | | | | | | |
| | activator | | | | | | | | |
| X89399 | RAS p21 | 0.25 | 0.10 | 8.5 | 14.9 | 4.8 | 18.6 | 4.3 | 4.8 |
| | protein | | | | | | | | |
| | activator | | | | | | | | |
| AJ005273 | antigenic | 0.70 | 0.10 | 7.6 | 11.1 | 2.8 | 9.9 | 12.0 | 4.6 |
| | determinant of | | | | | | | | |
| | recA protein | | | | | | | | |
| AK001154 | hypothetical | 1.70 | 0.96 | 2.4 | 4.4 | 2.9 | 8.9 | 2.4 | 4.5 |
| | protein | | | | | | | | |
| AL133605 | unknown | 0.26 | 0.15 | 12.4 | 4.2 | 4.4 | 3.3 | 3.3 | 4.1 |
| U71092 | G protein- | 0.53 | 0.06 | 19.0 | 9.1 | 2.2 | 12.0 | 3.3 | 4.1 |
| | coupled | | | | | | | | |
| | receptor 24 | | | | | | | | |
| AF074723 | RNA | 0.67 | 0.54 | 4.0 | 3.2 | 3.1 | 3.4 | 6.0 | 4.0 |
| | polymerase II | | | | | | | | |
| | transcriptional | | | | | | | | |
| | regulation | | | | | | | | |
| | mediator | | | | | | | | |
| AL137577 | unknown | 0.32 | 0.12 | 31.4 | 6.2 | 5.3 | 10.1 | 25.3 | 3.9 |
| AF151043 | hypothetical | 0.48 | 0.35 | 2.6 | 2.2 | 2.0 | 3.3 | 2.2 | 3.8 |
| | protein | | | | | | | | |
| AF131831 | unknown | 0.67 | 0.81 | 2.1 | 7.0 | 3.5 | 3.2 | 3.9 | 3.7 |
| D50405 | histone | 1.52 | 2.62 | 3.1 | 7.2 | 2.9 | 4.1 | 2.8 | 3.7 |
| | deacetylase 1 | | | | | | | | |
| U78305 | protein | 1.21 | 0.20 | 4.7 | 13.0 | 3.5 | 5.9 | 4.2 | 3.7 |
| | phosphatase | | | | | | | | |
| | 1D | | | | | | | | |
| AL035562 | paired box | 0.24 | 0.01 | 30.2 | 81.9 | 5.6 | 82.3 | 6.2 | 3.7 |
| | gene 1 | | | | | | | | |
| U67156 | mitogen- | 1.15 | 0.30 | 6.6 | 3.0 | 2.2 | 2.3 | 2.5 | 3.6 |
| | activated | | | | | | | | |
| | protein kinase | | | | | | | | |
| | kinase kinase 5 | | | | | | | | |
| AL031121 | unknown | 0.24 | 0.09 | 5.2 | 3.7 | 2.3 | 6.5 | 9.1 | 3.6 |
| U13666 | G protein- | 0.34 | 0.14 | 3.8 | 5.4 | 3.1 | 3.3 | 2.8 | 3.6 |
| | coupled | | | | | | | | |
| | receptor 1 | | | | | | | | |
| AB018285 | KIAA0742 | 0.53 | 0.13 | 14.9 | 13.9 | 5.9 | 18.5 | 15.2 | 3.5 |
| | protein | | | | | | | | |
| D42053 | site-1 protease | 0.63 | 0.40 | 2.6 | 7.1 | 5.6 | 9.2 | 2.6 | 3.5 |
| AK001135 | Sec23- | 0.29 | 0.53 | 5.7 | 4.5 | 3.4 | 2.6 | 11.3 | 3.4 |
| | interacting | | | | | | | | |
| | protein p125 | | | | | | | | |
| AL137461 | unknown | 0.25 | 0.02 | 23.8 | 9.0 | 2.7 | 59.2 | 12.5 | 3.3 |
| NM_006963 | zinc finger | 0.10 | 0.08 | 3.2 | 7.6 | 3.7 | 7.9 | 11.2 | 3.2 |
| | protein 22 | | | | | | | | |
| AL137540 | unknown | 0.67 | 0.79 | 3.9 | 2.6 | 5.6 | 4.2 | 3.5 | 3.1 |
| AL137718 | unknown | 0.95 | 0.18 | 4.7 | 8.0 | 4.0 | 13.3 | 3.0 | 3.1 |
| AF012086 | RAN binding | 1.20 | 0.59 | 4.6 | 4.0 | 2.0 | 4.6 | 3.6 | 3.1 |
| | protein 2-like 1 | | | | | | | | |
| S57296 | HER2/neu | 0.59 | 0.17 | 7.3 | 12.1 | 2.3 | 20.0 | 22.2 | 3.0 |
| | receptor | | | | | | | | |
| NM_013329 | GC-rich | 0.16 | 0.08 | 6.9 | 14.3 | 9.7 | 3.3 | 7.2 | 3.0 |
| | sequence | | | | | | | | |
| | DNA-binding | | | | | | | | |
| | factor | | | | | | | | |
| | candidate | | | | | | | | |
| AF038664 | UDP-Gal:beta | 0.15 | 0.03 | 13.4 | 22.2 | 5.4 | 15.8 | 17.6 | 3.0 |
| | GlcNAc beta | | | | | | | | |
| | 1_4- | | | | | | | | |
| | galactosyltransferase | | | | | | | | |
| AF080579 | Homo sapiens | 0.34 | 1.03 | 3.3 | 3.0 | 6.7 | 2.1 | 2.9 | 2.9 |
| | integral | | | | | | | | |
| | membrane | | | | | | | | |
| | protein | | | | | | | | |
| AK001075 | hypothetical | 0.67 | 0.10 | 2.1 | 2.6 | 2.6 | 8.9 | 2.2 | 2.9 |
| | protein | | | | | | | | |
| AB011124 | KIAA0552 | 0.46 | 0.04 | 9.6 | 72.0 | 6.0 | 33.9 | 13.6 | 2.9 |
| | gene product | | | | | | | | |
| J03068 | N- | 0.54 | 0.21 | 2.2 | 5.0 | 2.4 | 5.2 | 3.6 | 2.8 |
| | acylaminoacyl- | | | | | | | | |
| | peptide | | | | | | | | |
| | hydrolase | | | | | | | | |
| D87120 | osteoblast | 0.87 | 0.87 | 2.2 | 2.0 | 4.7 | 2.3 | 2.0 | 2.8 |
| | protein | | | | | | | | |
| AB006537 | IL-1R | 0.17 | 0.07 | 2.9 | 7.0 | 14.5 | 5.3 | 6.6 | 2.8 |
| | accessory | | | | | | | | |
| | protein | | | | | | | | |
| L34587 | transcription | 2.49 | 1.23 | 2.2 | 16.3 | 5.0 | 15.8 | 5.5 | 2.7 |
| | elongation | | | | | | | | |
| | factor B | | | | | | | | |
| D31891 | SET domain_bifurcated_1 | 1.02 | 0.29 | 3.9 | 6.0 | 4.3 | 4.9 | 6.6 | 2.7 |
| D00760 | proteasome | 4.97 | 4.94 | 4.1 | 2.6 | 2.0 | 2.8 | 2.7 | 2.7 |
| | subunit_alpha | | | | | | | | |
| | type_2 | | | | | | | | |
| AC004774 | distal-less | 0.25 | 0.12 | 2.3 | 6.3 | 3.8 | 5.2 | 5.2 | 2.6 |
| | homeo box 5 | | | | | | | | |
| AL024493 | unknown | 1.46 | 0.54 | 4.8 | 13.5 | 2.1 | 11.6 | 6.8 | 2.6 |
| AB014536 | copine III | 1.80 | 1.29 | 3.2 | 9.5 | 3.8 | 6.8 | 2.6 | 2.6 |
| X59770 | IL-1R type II | 0.59 | 0.16 | 9.6 | 4.7 | 3.9 | 3.2 | 4.9 | 2.5 |
| AF052183 | unknown | 0.65 | 0.76 | 4.0 | 3.7 | 2.3 | 5.0 | 3.0 | 2.5 |
| AK000541 | hypothetical | 0.92 | 0.27 | 4.5 | 13.9 | 3.6 | 18.1 | 4.3 | 2.5 |
| | protein | | | | | | | | |
| U88528 | cAMP | 1.37 | 0.86 | 3.1 | 5.4 | 2.1 | 2.8 | 2.1 | 2.4 |
| | responsive | | | | | | | | |
| | element | | | | | | | | |
| | binding protein | | | | | | | | |
| M97925 | defensin alpha | 0.33 | 0.07 | 4.6 | 35.9 | 2.0 | 7.8 | 6.5 | 2.4 |
| | 5_Paneth cell- | | | | | | | | |
| | specific | | | | | | | | |
| NM_013393 | cell division | 1.38 | 0.94 | 3.1 | 5.8 | 2.1 | 4.2 | 2.6 | 2.3 |
| | protein FtsJ | | | | | | | | |
| X62744 | MHC class II | 0.86 | 0.32 | 4.0 | 4.7 | 2.3 | 2.9 | 6.1 | 2.3 |
| | DM alpha | | | | | | | | |
| AF251040 | putative | 0.64 | 0.30 | 6.7 | 3.4 | 2.9 | 3.9 | 5.7 | 2.2 |
| | nuclear protein | | | | | | | | |
| AK000227 | hypothetical | 1.49 | 0.43 | 3.4 | 7.1 | 2.3 | 3.3 | 9.1 | 2.1 |
| | protein | | | | | | | | |
| U88666 | SFRS protein | 1.78 | 0.37 | 3.4 | 5.9 | 2.6 | 8.4 | 6.1 | 2.0 |
| | kinase 2 | | | | | | | | |

The peptides at a concentration of 50 μg/ml were shown to increase the expression of many polynucleotides.
Peptide was incubated with the human A549 epithelial cells for 4 h and the RNA was isolated, converted into labeled cDNA probes and hybridized to Human Operon arrays (PRHU04).
The intensity of polynucleotides in control, unstimulated cells are shown in the second and third columns for labeling of cDNA with the dyes Cy3 and Cy5 respectively.
The “ID#: Control” columns refer to the intensity of polynucleotide expression in peptide-simulated cells divided by the intensity of unstimulated cells.

TABLE 35
Up-regulation of Polynucleotide expression in A549 cells induced by Formula E
Peptides.

| Accession | | ctrl- | ctrl- | | | | | | |
|---|---|---|---|---|---|---|---|---|---|
| Number | Gene | Cy3 | Cy5 | ID 33:ctrl | ID 34:ctrl | ID 35:ctrl | ID 36:ctrl | ID 37:ctrl | ID 38:ctrl |
| AL049689 | Novel human | 0.25 | 0.05 | 2.7 | 26.5 | 3.3 | 21.7 | 5.4 | 37.9 |
| | mRNA | | | | | | | | |
| AK000576 | hypothetical | 0.27 | 0.06 | 3.0 | 19.1 | 3.9 | 23.0 | 3.1 | 28.3 |
| | protein | | | | | | | | |
| X74837 | mannosidase, | 0.10 | 0.07 | 5.6 | 10.0 | 10.8 | 12.3 | 12.0 | 19.9 |
| | alpha class | | | | | | | | |
| | 1A member 1 | | | | | | | | |
| AK000258 | hypothetical | 0.27 | 0.07 | 14.0 | 11.1 | 7.9 | 16.1 | 6.2 | 18.9 |
| | protein | | | | | | | | |
| X89067 | transient | 0.20 | 0.14 | 3.7 | 2.2 | 2.4 | 2.6 | 8.0 | 18.1 |
| | receptor | | | | | | | | |
| AL137619 | unknown | 0.16 | 0.08 | 6.3 | 6.7 | 10.8 | 10.5 | 7.9 | 16.5 |
| NM_003445 | zinc finger | 0.17 | 0.07 | 4.0 | 23.6 | 2.9 | 13.6 | 4.3 | 14.4 |
| | protein | | | | | | | | |
| X03084 | complement | 0.36 | 0.15 | 2.4 | 3.1 | 2.9 | 7.7 | 3.4 | 13.7 |
| | component 1 | | | | | | | | |
| U27330 | fucosyltransferase 5 | 0.39 | 0.08 | 2.4 | 2.5 | 2.6 | 12.1 | 3.5 | 13.0 |
| AF070549 | unknown | 0.16 | 0.09 | 2.7 | 4.7 | 7.9 | 10.3 | 4.2 | 12.6 |
| AB020335 | sel-1-like | 0.19 | 0.24 | 2.9 | 2.6 | 2.0 | 7.3 | 4.7 | 12.4 |
| M26901 | renin | 0.09 | 0.12 | 14.9 | 2.2 | 7.3 | 12.0 | 20.8 | 12.0 |
| Y07828 | ring finger | 0.09 | 0.06 | 9.0 | 26.6 | 8.9 | 16.0 | 3.6 | 11.6 |
| | protein | | | | | | | | |
| AK001848 | hypothetical | 0.21 | 0.07 | 6.2 | 8.2 | 2.7 | 5.2 | 5.5 | 10.9 |
| | protein | | | | | | | | |
| NM_016331 | zinc finger | 0.16 | 0.08 | 7.6 | 5.1 | 7.0 | 25.5 | 5.5 | 10.9 |
| | protein | | | | | | | | |
| U75330 | neural cell | 0.42 | 0.08 | 2.5 | 3.6 | 2.0 | 5.8 | 6.2 | 9.9 |
| | adhesion | | | | | | | | |
| | molecule 2 | | | | | | | | |
| AB037826 | unknown | 0.16 | 0.11 | 3.8 | 6.0 | 3.4 | 13.4 | 6.0 | 9.8 |
| M34041 | adrenergic | 0.30 | 0.13 | 4.5 | 4.5 | 3.7 | 8.6 | 5.6 | 9.8 |
| | alpha-2B- | | | | | | | | |
| | receptor | | | | | | | | |
| D38449 | putative G | 0.18 | 0.09 | 2.3 | 25.8 | 11.7 | 2.3 | 3.2 | 9.5 |
| | protein | | | | | | | | |
| | coupled | | | | | | | | |
| | receptor | | | | | | | | |
| AJ250562 | transmembrane | 0.13 | 0.10 | 10.0 | 8.4 | 2.2 | 8.1 | 16.3 | 9.1 |
| | 4 superfamily | | | | | | | | |
| | member 2 | | | | | | | | |
| AK001807 | hypothetical | 0.18 | 0.12 | 4.2 | 5.3 | 4.6 | 3.2 | 4.0 | 8.3 |
| | protein | | | | | | | | |
| AL133051 | unknown | 0.09 | 0.07 | 5.1 | 13.6 | 6.0 | 9.1 | 2.2 | 8.2 |
| U43843 | Neuro-d4 | 0.61 | 0.10 | 2.0 | 6.4 | 2.3 | 16.6 | 2.2 | 8.1 |
| | homolog | | | | | | | | |
| NM_013227 | aggrecan 1 | 0.28 | 0.15 | 7.5 | 3.1 | 2.5 | 6.9 | 8.5 | 7.8 |
| AF226728 | somatostatin | 0.23 | 0.17 | 7.0 | 3.6 | 3.1 | 5.5 | 3.5 | 7.7 |
| | receptor- | | | | | | | | |
| | interacting | | | | | | | | |
| | protein | | | | | | | | |
| AK001024 | guanine | 0.16 | 0.11 | 3.9 | 12.3 | 2.7 | 7.4 | 3.3 | 7.0 |
| | nucleotide- | | | | | | | | |
| | binding | | | | | | | | |
| | protein | | | | | | | | |
| AC002302 | unknown | 0.13 | 0.14 | 16.1 | 5.8 | 5.8 | 2.6 | 9.6 | 6.2 |
| AB007958 | unknown | 0.17 | 0.27 | 2.0 | 2.3 | 11.3 | 3.3 | 3.0 | 6.1 |
| AF059293 | cytokine | 0.19 | 0.22 | 3.6 | 2.5 | 10.2 | 3.8 | 2.7 | 5.9 |
| | receptor-like | | | | | | | | |
| | factor 1 | | | | | | | | |
| V01512 | v-fos | 0.27 | 0.21 | 6.7 | 3.7 | 13.7 | 9.3 | 3.7 | 5.4 |
| U82762 | sialyltransferase 8 | 0.23 | 0.15 | 3.2 | 6.5 | 2.7 | 9.2 | 5.7 | 5.4 |
| U44059 | thyrotrophic | 0.05 | 0.13 | 22.9 | 7.1 | 12.5 | 7.4 | 9.7 | 5.4 |
| | embryonic | | | | | | | | |
| | factor | | | | | | | | |
| X05323 | antigen | 0.39 | 0.13 | 4.3 | 2.5 | 2.2 | 7.4 | 2.8 | 5.1 |
| | identified by | | | | | | | | |
| | monoclonal | | | | | | | | |
| | antibody | | | | | | | | |
| U72671 | ICAM 5, | 0.25 | 0.14 | 5.3 | 2.7 | 3.7 | 10.0 | 3.2 | 4.8 |
| AL133626 | hypothetical | 0.26 | 0.25 | 2.2 | 4.2 | 2.9 | 3.0 | 2.6 | 4.7 |
| | protein | | | | | | | | |
| X96401 | MAX | 0.31 | 0.29 | 6.9 | 2.3 | 4.9 | 3.1 | 2.9 | 4.6 |
| | binding | | | | | | | | |
| | protein | | | | | | | | |
| AL117533 | unknown | 0.05 | 0.26 | 8.2 | 2.7 | 11.1 | 2.5 | 11.9 | 4.5 |
| AK001550 | hypothetical | 0.10 | 0.30 | 8.0 | 2.0 | 4.9 | 2.1 | 7.8 | 4.5 |
| | protein | | | | | | | | |
| AB032436 | Homo | 0.14 | 0.21 | 5.1 | 2.2 | 9.1 | 4.5 | 6.4 | 4.4 |
| | sapiens BNPI | | | | | | | | |
| | mRNA | | | | | | | | |
| AL035447 | hypothetical | 0.28 | 0.23 | 4.3 | 3.7 | 8.7 | 5.2 | 3.7 | 4.2 |
| | protein | | | | | | | | |
| U09414 | zinc finger | 0.28 | 0.25 | 4.0 | 2.2 | 4.7 | 3.3 | 7.2 | 4.2 |
| | protein | | | | | | | | |
| AK001256 | unknown | 0.09 | 0.08 | 5.3 | 6.5 | 31.1 | 12.7 | 6.4 | 4.1 |
| L14813 | carboxyl | 0.64 | 0.21 | 2.7 | 6.2 | 3.1 | 2.1 | 3.4 | 3.9 |
| | ester lipase- | | | | | | | | |
| | like | | | | | | | | |
| AF038181 | unknowan | 0.06 | 0.18 | 34.1 | 6.4 | 4.5 | 8.7 | 11.3 | 3.9 |
| NM_001486 | glucokinase | 0.21 | 0.08 | 3.0 | 2.2 | 6.5 | 12.4 | 5.7 | 3.9 |
| AB033000 | hypothetical | 0.24 | 0.22 | 3.4 | 3.3 | 7.1 | 5.5 | 4.5 | 3.8 |
| | protein | | | | | | | | |
| AL117567 | DKFZP566O | 0.44 | 0.22 | 2.2 | 2.7 | 3.9 | 4.0 | 4.5 | 3.7 |
| | 084 protein | | | | | | | | |
| NM_012126 | carbohydrate | 0.31 | 0.20 | 5.5 | 5.4 | 3.8 | 5.5 | 2.6 | 3.5 |
| | sulfotransferase 5 | | | | | | | | |
| AL031687 | unknown | 0.16 | 0.27 | 5.9 | 2.6 | 3.4 | 2.3 | 4.9 | 3.5 |
| X04506 | apolipoprotein B | 0.29 | 0.32 | 5.4 | 4.4 | 6.9 | 5.5 | 2.1 | 3.5 |
| NM_006641 | CCR9 | 0.35 | 0.11 | 3.3 | 3.3 | 2.2 | 16.5 | 2.3 | 3.5 |
| Y00970 | acrosin | 0.12 | 0.14 | 8.2 | 8.8 | 3.1 | 6.2 | 17.5 | 3.4 |
| X67098 | rTS beta | 0.19 | 0.26 | 2.4 | 3.1 | 7.8 | 3.5 | 4.4 | 3.3 |
| | protein | | | | | | | | |
| U51990 | pre-mRNA | 0.56 | 0.19 | 2.2 | 3.0 | 2.8 | 13.7 | 2.9 | 3.0 |
| | splicing | | | | | | | | |
| | factor | | | | | | | | |
| AF030555 | fatty-acid- | 0.10 | 0.39 | 3.5 | 6.9 | 13.3 | 4.4 | 7.5 | 2.9 |
| | Coenzyme A | | | | | | | | |
| AL009183 | TNFR | 0.46 | 0.19 | 6.0 | 4.1 | 2.8 | 8.6 | 2.6 | 2.8 |
| | superfamily, | | | | | | | | |
| | member 9 | | | | | | | | |
| AF045941 | sciellin | 0.16 | 0.21 | 11.6 | 2.4 | 2.8 | 2.2 | 4.1 | 2.8 |
| AF072756 | A kinase | 0.33 | 0.07 | 2.5 | 5.3 | 3.9 | 32.7 | 2.3 | 2.7 |
| | anchor | | | | | | | | |
| | protein 4 | | | | | | | | |
| X78678 | ketohexokinase | 0.10 | 0.20 | 18.0 | 3.5 | 4.1 | 2.5 | 14.6 | 2.6 |
| AL031734 | unknown | 0.03 | 0.39 | 43.7 | 2.3 | 41.7 | 4.0 | 10.8 | 2.5 |
| D87717 | KIAA0013 | 0.35 | 0.42 | 4.2 | 2.3 | 3.6 | 2.6 | 2.9 | 2.5 |
| | gene product | | | | | | | | |
| U01824 | solute carrier | 0.42 | 0.29 | 4.8 | 2.3 | 4.2 | 7.1 | 4.2 | 2.4 |
| | family 1 | | | | | | | | |
| AF055899 | solute carrier | 0.14 | 0.31 | 9.5 | 12.3 | 7.4 | 4.7 | 6.6 | 2.3 |
| | family 27 | | | | | | | | |
| U22526 | lanosterol | 0.09 | 0.45 | 4.1 | 3.4 | 10.4 | 2.2 | 17.9 | 2.3 |
| | synthase | | | | | | | | |
| AB032963 | unknown | 0.19 | 0.34 | 6.3 | 6.1 | 2.9 | 2.1 | 5.7 | 2.2 |
| NM_015974 | lambda- | 0.17 | 0.25 | 11.4 | 2.8 | 5.9 | 2.4 | 5.8 | 2.2 |
| | crystallin | | | | | | | | |
| X82200 | stimulated | 0.23 | 0.15 | 8.2 | 3.4 | 3.0 | 2.8 | 11.3 | 2.2 |
| | trans-acting | | | | | | | | |
| | factor | | | | | | | | |
| AL137522 | unknown | 0.12 | 0.26 | 12.1 | 3.7 | 12.6 | 6.9 | 4.3 | 2.2 |
| Z99916 | crystallin, | 0.28 | 0.65 | 2.5 | 2.1 | 3.6 | 2.2 | 2.6 | 2.1 |
| | beta B3 | | | | | | | | |
| AF233442 | ubiquitin | 0.41 | 0.31 | 2.6 | 3.6 | 3.6 | 4.5 | 3.4 | 2.1 |
| | specific | | | | | | | | |
| | protease 21 | | | | | | | | |
| AK001927 | hypothetical | 0.24 | 0.52 | 7.6 | 5.6 | 5.0 | 2.5 | 4.1 | 2.0 |
| | protein | | | | | | | | |

The peptides at a concentration of 50 μg/ml were shown to increase the expression of many polynucleotides.
Peptide was incubated with the human A549 epithelial cells for 4 h and the RNA was isolated, converted into labeled cDNA probes and hybridized to Human Operon arrays (PRHU04).
The intensity of polynucleotides in control, unstimulated cells are shown in the second and third columns for labeling of cDNA with the dyes Cy3 and Cy5 respectively.
The “ID#: Control” columns refer to the intensity of polynucleotide expression in peptide-simulated cells divided by the intensity of unstimulated cells.

TABLE 36
Up-regulation of Polynucleotide expression in A549 cells induced by Formula F
Peptides.

| Accession | | ctrl- | ctrl- | Ratio | Ratio | Ratio | Ratio | Ratio |
|---|---|---|---|---|---|---|---|---|
| Number | Gene | Cy3 | Cy5 | ID 40:ctrl | ID 42:ctrl | ID 43:ctrl | ID 44:ctrl | ID 45:ctrl |
| AF025840 | polymerase | 0.34 | 0.96 | 3.4 | 2.0 | 2.0 | 2.1 | 4.3 |
| | epsilon 2 | | | | | | | |
| AF132495 | CGI-133 | 0.83 | 0.67 | 3.0 | 2.2 | 2.6 | 2.8 | 5.1 |
| | protein | | | | | | | |
| AL137682 | hypothetical | 0.73 | 0.40 | 2.0 | 5.3 | 4.8 | 2.9 | 8.2 |
| | protein | | | | | | | |
| U70426 | regulator of | 0.23 | 0.25 | 3.1 | 3.0 | 5.3 | 3.1 | 12.2 |
| | G-protein | | | | | | | |
| | signaling 16 | | | | | | | |
| AK001135 | Sec23- | 0.29 | 0.53 | 3.2 | 2.6 | 3.3 | 14.4 | 5.2 |
| | interacting | | | | | | | |
| | protein | | | | | | | |
| | p125 | | | | | | | |
| AB023155 | KIAA0938 | 0.47 | 0.21 | 2.7 | 4.8 | 8.1 | 4.2 | 10.4 |
| | protein | | | | | | | |
| AB033080 | cell cycle | 0.31 | 0.31 | 4.4 | 2.2 | 5.9 | 4.3 | 6.9 |
| | progression | | | | | | | |
| | 8 protein | | | | | | | |
| AF061836 | Ras | 0.29 | 0.31 | 3.2 | 2.5 | 11.1 | 18.8 | 6.8 |
| | association | | | | | | | |
| | domain | | | | | | | |
| | family 1 | | | | | | | |
| AK000298 | hypothetical | 0.48 | 0.27 | 3.3 | 2.2 | 7.1 | 5.6 | 7.7 |
| | protein | | | | | | | |
| L75847 | zinc finger | 0.35 | 0.52 | 3.2 | 3.0 | 4.0 | 3.0 | 3.9 |
| | protein | | | | | | | |
| X97267 | protein | 0.19 | 0.24 | 4.1 | 9.3 | 2.4 | 4.2 | 8.3 |
| | tyrosine | | | | | | | |
| | phosphatase | | | | | | | |
| Z11933 | POU | 0.09 | 0.23 | 8.7 | 2.5 | 3.6 | 4.3 | 8.2 |
| | domain | | | | | | | |
| | class 3 TF 2 | | | | | | | |
| AB037744 | unknown | 0.37 | 0.57 | 2.6 | 2.9 | 2.7 | 3.0 | 3.1 |
| U90908 | unknown | 0.12 | 0.16 | 11.8 | 7.7 | 3.4 | 7.8 | 11.2 |
| AL050139 | unknown | 0.29 | 0.60 | 5.2 | 2.4 | 3.3 | 3.0 | 2.8 |
| AB014615 | fibroblast | 0.19 | 0.07 | 5.4 | 3.5 | 8.5 | 3.2 | 22.7 |
| | growth | | | | | | | |
| | factor 8 | | | | | | | |
| M28825 | CD1A | 0.51 | 0.36 | 4.1 | 2.6 | 2.0 | 4.6 | 4.4 |
| | antigen | | | | | | | |
| U27330 | fucosyltransferase 5 | 0.39 | 0.08 | 3.3 | 2.1 | 24.5 | 8.2 | 19.3 |
| NM_00696 | zinc finger | 0.10 | 0.08 | 10.4 | 12.6 | 12.3 | 29.2 | 20.5 |
| | protein | | | | | | | |
| AF093670 | peroxisomal | 0.44 | 0.53 | 4.0 | 2.6 | 2.6 | 4.3 | 2.9 |
| | biogenesis | | | | | | | |
| | factor | | | | | | | |
| AK000191 | hypothetical | 0.50 | 0.182 | 2.3 | 3.6 | 4.4 | 2.2 | 8.2 |
| | protein | | | | | | | |
| AB022847 | unknown | 0.39 | 0.24 | 2.1 | 6.9 | 4.5 | 2.8 | 6.2 |
| AK000358 | microfibrillar- | 0.28 | 0.28 | 5.7 | 2.0 | 3.5 | 5.2 | 5.2 |
| | associated | | | | | | | |
| | protein 3 | | | | | | | |
| X74837 | mannosidase_alpha | 0.10 | 0.07 | 13.1 | 18.4 | 23.6 | 16.3 | 20.8 |
| | class 1A | | | | | | | |
| AF053712 | TNF | 0.17 | 0.08 | 11.3 | 9.3 | 13.4 | 10.6 | 16.6 |
| | superfamily_member | | | | | | | |
| | 11 | | | | | | | |
| AL133114 | DKFZP586 | 0.11 | 0.32 | 8.5 | 3.4 | 4.9 | 5.3 | 4.3 |
| | P2421 | | | | | | | |
| | protein | | | | | | | |
| AF049703 | E74-like | 0.22 | 0.24 | 5.1 | 6.0 | 3.3 | 2.7 | 5.4 |
| | factor 5 | | | | | | | |
| AL137471 | hypothetical | 0.29 | 0.05 | 4.0 | 15.0 | 10.1 | 2.7 | 25.3 |
| | protein | | | | | | | |
| AL035397 | unknown | 0.33 | 0.14 | 2.3 | 2.8 | 10.6 | 4.6 | 9.3 |
| AL035447 | hypothetical | 0.28 | 0.23 | 3.8 | 6.8 | 2.7 | 3.0 | 5.7 |
| | protein | | | | | | | |
| X55740 | CD73 | 0.41 | 0.61 | 2.1 | 3.3 | 2.9 | 3.2 | 2.1 |
| NM_004909 | taxol | 0.20 | 0.22 | 3.9 | 2.9 | 6.5 | 3.2 | 5.6 |
| | resistance | | | | | | | |
| | associated | | | | | | | |
| | gene 3 | | | | | | | |
| AF233442 | ubiquitin | 0.41 | 0.31 | 2.9 | 4.7 | 2.7 | 3.5 | 3.9 |
| | specific | | | | | | | |
| | protease | | | | | | | |
| U92980 | unknown | 0.83 | 0.38 | 4.2 | 4.1 | 4.8 | 2.3 | 3.1 |
| AF105424 | myosin | 0.30 | 0.22 | 2.8 | 3.3 | 4.4 | 2.3 | 5.3 |
| | heavy | | | | | | | |
| | polypeptide- | | | | | | | |
| | like | | | | | | | |
| M26665 | histatin 3 | 0.29 | 0.26 | 7.9 | 3.5 | 4.6 | 3.5 | 4.5 |
| AF083898 | neuro- | 0.20 | 0.34 | 18.7 | 3.8 | 2.2 | 3.6 | 3.5 |
| | oncological | | | | | | | |
| | ventral | | | | | | | |
| | antigen 2 | | | | | | | |
| AJ009771 | ariadne_Drosophila_homolog | 0.33 | 0.06 | 2.3 | 17.6 | 15.9 | 2.5 | 20.3 |
| | of | | | | | | | |
| AL022393 | hypothetical | 0.05 | 0.33 | 32.9 | 2.4 | 3.0 | 69.4 | 3.4 |
| | protein P1 | | | | | | | |
| AF039400 | chloride | 0.11 | 0.19 | 8.4 | 2.9 | 5.1 | 18.1 | 5.9 |
| | channel_calcium | | | | | | | |
| | activated_family | | | | | | | |
| | member 1 | | | | | | | |
| AJ012008 | dimethylarginine | 0.42 | 0.43 | 5.1 | 3.3 | 3.2 | 6.2 | 2.6 |
| | dimethylaminohydrolase 2 | | | | | | | |
| AK000542 | hypothetical | 0.61 | 0.24 | 2.1 | 4.5 | 5.0 | 3.7 | 4.4 |
| | protein | | | | | | | |
| AL133654 | unknown | 0.27 | 0.40 | 2.8 | 2.1 | 2.5 | 2.5 | 2.6 |
| AL137513 | unknown | 0.43 | 0.43 | 6.4 | 3.2 | 3.8 | 2.3 | 2.3 |
| U05227 | GTP- | 0.38 | 0.36 | 5.0 | 3.1 | 3.1 | 2.2 | 2.8 |
| | binding | | | | | | | |
| | protein | | | | | | | |
| D38449 | putative G | 0.18 | 0.09 | 5.8 | 6.7 | 6.7 | 9.1 | 10.4 |
| | protein | | | | | | | |
| | coupled | | | | | | | |
| | receptor | | | | | | | |
| U80770 | unknown | 0.31 | 0.14 | 3.9 | 3.8 | 6.6 | 3.1 | 6.8 |
| X61177 | IL-5R alpha | 0.40 | 0.27 | 2.6 | 4.4 | 9.8 | 8.1 | 3.6 |
| U35246 | vacuolar | 0.15 | 0.42 | 5.8 | 2.8 | 2.6 | 4.5 | 2.2 |
| | protein | | | | | | | |
| | sorting 45A | | | | | | | |
| AB017016 | brain- | 0.27 | 0.29 | 6.0 | 2.6 | 3.4 | 3.1 | 3.1 |
| | specific | | | | | | | |
| | protein p25 | | | | | | | |
| | alpha | | | | | | | |
| X82153 | cathepsin K | 0.45 | 0.20 | 4.2 | 5.2 | 4.8 | 4.4 | 4.6 |
| AC005162 | probable | 0.12 | 0.28 | 11.9 | 3.4 | 6.8 | 18.7 | 3.2 |
| | carboxypeptidase | | | | | | | |
| | precursor | | | | | | | |
| AL137502 | unknown | 0.22 | 0.16 | 3.9 | 4.9 | 7.3 | 3.9 | 5.3 |
| U66669 | 3- | 0.30 | 0.40 | 10.3 | 3.5 | 5.2 | 2.3 | 2.1 |
| | hydroxyisobutyryl- | | | | | | | |
| | Coenzyme | | | | | | | |
| | A hydrolase | | | | | | | |
| AK000102 | unknown | 0.39 | 0.30 | 2.8 | 5.3 | 5.2 | 4.1 | 2.8 |
| AF034970 | docking | 0.28 | 0.05 | 3.3 | 8.5 | 15.7 | 4.0 | 17.3 |
| | protein 2 | | | | | | | |
| AK000534 | hypothetical | 0.13 | 0.29 | 6.8 | 2.3 | 4.0 | 20.6 | 2.9 |
| | protein | | | | | | | |
| J04599 | biglycan | 0.39 | 0.30 | 4.0 | 3.7 | 4.0 | 4.8 | 2.8 |
| AL133612 | unknown | 0.62 | 0.33 | 2.7 | 3.4 | 5.2 | 3.0 | 2.5 |
| D10495 | protein | 0.18 | 0.10 | 12.0 | 20.7 | 8.7 | 6.8 | 8.1 |
| | kinase C | | | | | | | |
| | delta | | | | | | | |
| X58467 | cytochrome | 0.07 | 0.24 | 15.4 | 4.7 | 7.9 | 34.4 | 3.4 |
| | P450 | | | | | | | |
| AF131806 | unknown | 0.31 | 0.25 | 2.6 | 3.4 | 5.7 | 7.0 | 3.2 |
| AK000351 | hypothetical | 0.34 | 0.13 | 4.0 | 6.9 | 5.5 | 2.8 | 6.3 |
| | protein | | | | | | | |
| AF075050 | hypothetical | 0.55 | 0.09 | 2.7 | 17.8 | 5.1 | 2.2 | 8.3 |
| | protein | | | | | | | |
| AK000566 | hypothetical | 0.15 | 0.35 | 6.7 | 2.2 | 6.8 | 6.4 | 2.1 |
| | protein | | | | | | | |
| | unknown | | | | | | | |
| U43328 | cartilage | 0.44 | 0.19 | 2.5 | 6.2 | 6.9 | 7.8 | 3.8 |
| | linking | | | | | | | |
| | protein 1 | | | | | | | |
| AF045941 | sciellin | 0.16 | 0.21 | 6.8 | 7.5 | 4.8 | 6.9 | 3.4 |
| U27655 | regulator of | 0.24 | 0.29 | 5.5 | 4.9 | 2.9 | 4.9 | 2.4 |
| | G-protein | | | | | | | |
| | signaling 3 | | | | | | | |
| AK000058 | hypothetical | 0.25 | 0.15 | 5.0 | 9.7 | 16.4 | 2.7 | 4.5 |
| | protein | | | | | | | |
| AL035364 | hypothetical | 0.32 | 0.26 | 4.4 | 4.2 | 7.3 | 2.8 | 2.6 |
| | protein | | | | | | | |
| AK001864 | unknown | 0.40 | 0.25 | 3.7 | 3.7 | 4.6 | 3.2 | 2.6 |
| AB015349 | unknown | 0.14 | 0.24 | 10.5 | 2.8 | 3.7 | 8.0 | 2.7 |
| V00522 | MHC class | 0.62 | 0.22 | 4.8 | 3.9 | 4.7 | 2.5 | 3.0 |
| | II DR beta 3 | | | | | | | |
| U75330 | neural cell | 0.42 | 0.08 | 2.1 | 9.6 | 13.2 | 3.3 | 7.8 |
| | adhesion | | | | | | | |
| | molecule 2 | | | | | | | |
| NM_007199 | IL-1R- | 0.15 | 0.25 | 8.7 | 7.8 | 8.6 | 16.1 | 2.5 |
| | associated | | | | | | | |
| | kinase M | | | | | | | |
| D30742 | calcium/cal | 0.28 | 0.09 | 6.2 | 28.7 | 7.4 | 2.4 | 6.8 |
| | modulin- | | | | | | | |
| | dependent | | | | | | | |
| | protein | | | | | | | |
| | kinase IV | | | | | | | |
| X05978 | cystatin A | 0.63 | 0.17 | 2.7 | 4.8 | 9.4 | 2.2 | 3.6 |
| AF240467 | TLR-7 | 0.11 | 0.10 | 13.8 | 13.3 | 4.7 | 7.7 | 4.9 |

The peptides at a concentration of 50 μg/ml were shown to increase the expression of many polynucleotides.
Peptide was incubated with the human A549 epithelial cells for 4 h and the RNA was isolated, converted into labeled cDNA probes and hybridized to Human Operon arrays (PRHU04).
The intensity of polynucleotides in control, unstimulated cells are shown in the second and third columns for labeling of cDNA with the dyes Cy3 and CyS respectively.
The “Ratio ID#: Control” columns refer to the intensity of polynucleotide expression in peptide-simulated cells divided by the intensity of unstimulated cells.

TABLE 37
Up-regulation of Polynucleotide expression in A549 cells induced by Formula G
and additional Peptides.

| Accession | ctrl- | ctrl- | | | | | | | | |
|---|---|---|---|---|---|---|---|---|---|---|
| Number | Cy3 | Cy5 | ID 53:ctrl | ID 54:ctrl | ID 47:ctrl | ID 48:ctrl | ID 49:ctrl | ID 50:ctrl | ID 51:ctrl | ID 52:ctrl |
| U00115 | 0.51 | 0.07 | 27.4 | 7.3 | 2.4 | 3.1 | 4.8 | 8.3 | 3.5 | 20.0 |
| M91036 | 0.22 | 0.02 | 39.1 | 32.5 | 5.2 | 2.2 | 37.0 | 6.0 | 16.2 | 18.0 |
| AK000070 | 0.36 | 0.18 | 3.8 | 7.6 | 2.6 | 15.1 | 12.2 | 9.9 | 17.2 | 15.3 |
| AF055899 | 0.14 | 0.31 | 6.7 | 3.7 | 9.7 | 10.0 | 2.2 | 16.7 | 5.4 | 14.8 |
| AK001490 | 0.05 | 0.02 | 14.1 | 35.8 | 3.2 | 28.6 | 25.0 | 20.2 | 56.5 | 14.1 |
| X97674 | 0.28 | 0.28 | 3.2 | 3.7 | 4.0 | 10.7 | 3.3 | 3.1 | 4.0 | 13.2 |
| AB022847 | 0.39 | 0.24 | 4.1 | 4.4 | 4.5 | 2.7 | 3.7 | 10.4 | 5.0 | 11.3 |
| AJ275986 | 0.26 | 0.35 | 5.8 | 2.3 | 5.7 | 2.2 | 2.5 | 9.7 | 4.3 | 11.1 |
| D10495 | 0.18 | 0.10 | 8.0 | 3.4 | 4.6 | 2.0 | 6.9 | 2.5 | 12.7 | 10.3 |
| L36642 | 0.26 | 0.06 | 5.8 | 14.2 | 2.6 | 4.1 | 8.9 | 3.4 | 6.5 | 6.6 |
| M31166 | 0.31 | 0.12 | 4.8 | 3.8 | 12.0 | 3.6 | 9.8 | 2.4 | 8.8 | 6.4 |
| AF176012 | 0.45 | 0.26 | 3.1 | 2.9 | 2.8 | 2.6 | 2.3 | 6.9 | 3.0 | 5.8 |
| AF072756 | 0.33 | 0.07 | 9.9 | 9.3 | 4.4 | 4.3 | 3.2 | 4.9 | 11.9 | 5.4 |
| NM_014439 | 0.47 | 0.07 | 12.0 | 7.1 | 3.3 | 3.3 | 4.7 | 5.9 | 5.0 | 5.4 |
| AJ271351 | 0.46 | 0.12 | 3.4 | 3.5 | 2.3 | 4.7 | 2.3 | 2.7 | 6.9 | 5.2 |
| AK000576 | 0.27 | 0.06 | 7.4 | 15.7 | 2.9 | 4.7 | 9.0 | 2.4 | 8.2 | 5.1 |
| AJ272265 | 0.21 | 0.09 | 6.2 | 7.9 | 2.3 | 3.7 | 10.3 | 4.5 | 4.6 | 4.7 |
| AL122038 | 0.46 | 0.06 | 6.7 | 4.5 | 2.6 | 4.3 | 16.4 | 6.5 | 26.6 | 4.6 |
| AK000307 | 0.23 | 0.09 | 3.7 | 4.0 | 4.3 | 3.2 | 5.3 | 2.9 | 13.1 | 4.4 |
| AB029001 | 0.52 | 0.21 | 14.4 | 4.3 | 4.6 | 4.4 | 4.8 | 21.9 | 3.2 | 4.2 |
| U62437 | 0.38 | 0.13 | 12.6 | 6.5 | 4.2 | 6.7 | 2.2 | 3.7 | 4.8 | 3.9 |
| AF064854 | 0.15 | 0.16 | 2.6 | 2.9 | 6.2 | 8.9 | 14.4 | 5.0 | 9.1 | 3.9 |
| AL031588 | 0.40 | 0.26 | 8.3 | 5.2 | 2.8 | 3.3 | 5.3 | 9.0 | 5.6 | 3.4 |
| X89399 | 0.25 | 0.10 | 15.8 | 12.8 | 7.4 | 4.2 | 16.7 | 6.9 | 12.7 | 3.3 |
| D45399 | 0.21 | 0.18 | 3.0 | 4.7 | 3.3 | 4.4 | 8.7 | 5.3 | 5.1 | 3.3 |
| AB037716 | 0.36 | 0.40 | 5.1 | 7.5 | 2.6 | 2.1 | 3.5 | 3.1 | 2.4 | 2.8 |
| X79981 | 0.34 | 0.10 | 4.7 | 7.2 | 3.2 | 4.6 | 6.5 | 5.1 | 5.8 | 2.7 |
| AF034208 | 0.45 | 0.24 | 2.7 | 10.9 | 2.1 | 3.7 | 2.3 | 5.9 | 2.2 | 2.5 |
| AL133355 | 0.22 | 0.23 | 2.3 | 3.4 | 7.3 | 2.7 | 3.3 | 4.3 | 2.8 | 2.5 |
| NM_016281 | 0.40 | 0.19 | 6.6 | 10.6 | 2.1 | 2.8 | 5.0 | 11.2 | 10.6 | 2.5 |
| AF023614 | 0.11 | 0.42 | 2.2 | 2.2 | 6.0 | 7.5 | 5.0 | 2.7 | 2.0 | 2.4 |
| AF056717 | 0.43 | 0.62 | 4.3 | 3.2 | 5.1 | 4.0 | 4.6 | 9.7 | 3.1 | 2.2 |
| AB029039 | 0.79 | 0.49 | 2.7 | 3.3 | 3.7 | 2.0 | 2.3 | 2.4 | 4.8 | 2.2 |
| J03634 | 0.40 | 0.12 | 3.7 | 2.3 | 2.3 | 4.0 | 10.5 | 4.1 | 9.1 | 2.2 |
| U80764 | 0.31 | 0.18 | 2.3 | 7.4 | 4.2 | 2.3 | 5.1 | 3.3 | 8.8 | 2.1 |
| AB032963 | 0.19 | 0.34 | 4.0 | 7.3 | 5.0 | 3.0 | 2.9 | 6.7 | 3.8 | 2.1 |
| X82835 | 0.25 | 0.38 | 2.0 | 2.7 | 2.9 | 7.7 | 3.3 | 3.1 | 3.5 | 2.0 |
| The peptides at a concentration of 50 μg/ml were shown to increase the expression of many polynucleotides. | | | | | | | | | | |
| Peptide was incubated with the human A549 epithelial cells for 4 h and the RNA was isolated, converted into labelled cDNA probes and hybridised to Human Operon arrays (PRHU04). | | | | | | | | | | |
| The intensity of polynucleotides in control, unstimulated cells are shown in the second and third columns for labelling of cDNA with the dyes Cy3 and Cy5 respectively. | | | | | | | | | | |
| The “Ratio ID#: Control” columns refer to the intensity of polynucleotide expression in peptide-simulated cells divided by the intensity of unstimulated cells. | | | | | | | | | | |
| Accession numbers and gene designations are U00115, zinc finger protein; M91036, hemoglobin gamma G; K000070, hypothetical protein; AF055899, solute carrier family 27; AK001490, hypothetical protein; X97674, nuclear receptor coactivator 2; AB022847, unknown; AJ275986, transcription factor; D10495, protein kinase C, delta; L36642, EphA7; M31166, pentaxin-related gene; AF176012, | | | | | | | | | | |
| # unknown; AF072756, A kinase anchor protein 4; NM_014439, IL-1 Superfamily z; AJ271351, putative transcriptional regulator; AK000576, hypothetical protein; AJ272265, secreted phosphoprotein 2; AL122038, hypothetical protein; AK000307, hypothetical protein; AB029001, KIAA1078 protein; U62437, cholinergic receptor; AF064854, unknown; AL031588, hypothetical protein; X89399, RAS p21 protein activator; D45399, | | | | | | | | | | |
| # phosphodiesterase; AB037716, hypothetical protein; X79981, cadherin 5; AF034208, RIG-like 7-1; AL133355, chromosome 21 open reading frame 53; NM_016281, STE20-like kinase; AF023614, transmembrane activator and CAML interactor; AF056717, ash2-like; AB029039, KIAA1116 protein; J03634, inhibin, beta A; U80764, unknown; AB032963, unknown; X82835, sodium channel, voltage-gated, type IX. | | | | | | | | | | |

### EXAMPLE 5

### Induction of Chemokines in Cell Lines, Whole Human Blood, and in Mice by Peptides

The murine macrophage cell line RAW 264.7, THP-1 cells (human monocytes), a human epithelial cell line (A549), human bronchial epithelial cells (16HBEo14), and whole human blood were used. HBE cells were grown in MEM with Earle's. THP-1 cells were grown and maintained in RPMI 1640 medium. The RAW and A549 cell lines were maintained in DMEM supplemented with 10% fetal calf serum. The cells were seeded in 24 well plates at a density of 10⁶ cells per well in DMEM (see above) and A549 cells were seeded in 24 well plates at a density of 10⁵ cells per well in DMEM (see above) and both were incubated at 37° C. in 5% CO₂ overnight. DMEM was aspirated from cells grown overnight and replaced with fresh medium. After incubation of the cells with peptide, the release of chemokines into the culture supernatant was determined by ELISA (R&D Systems, Minneapolis, Minn.).

Animal studies were approved by the UBC Animal Care Committee.(UBC ACC # A01-0008). BALB/c mice were purchased from Charles River Laboratories and housed in standard animal facilities. Age, sex and weight matched adult mice were anaesthetized with an intraperitoneal injection of Avertin (4.4 mM 2-2-2-tribromoethanol, 2.5% 2-methyl-2-butanol, in distilled water), using 200 ill per 10 g body weight. The instillation was performed using a non-surgical, intratracheal instillation method adapted from Ho and Furst 1973. Briefly, the anaesthetized mouse was placed with its upper teeth hooked over a wire at the top of a support frame with its jaw held open and a spring pushing the thorax forward to position the pharynx, larynx and trachea in a vertical straight line. The airway was illuminated externally and an intubation catheter was inserted into the clearly illuminated tracheal lumen. Twenty-μl of peptide suspension or sterile water was placed in a well at the proximal end of the catheter and gently instilled into the trachea with 200 μl of air. The animals were maintained in an upright position for 2 minutes after instillation to allow the fluid to drain into the respiratory tree. After 4 hours the mice were euthanaised by intraperitoneal injection of 300 mg/kg of pentobarbital. The trachea was exposed; an intravenous catheter was passed into the proximal trachea and tied in place with suture thread. Lavage was performed by introducing 0.75 ml sterile PBS into the lungs via the tracheal cannula and then after a few seconds, withdrawing the fluid. This was repeated 3 times with the same sample of PBS. The lavage fluid was placed in a tube on ice and the total recovery volume per mouse was approximately 0.5 ml. The bronchoalveolar.lavage (BAL) fluid was centrifuged at 1200 rpm for 10 min, the clear supernatant removed and tested for TNF-α and MCP-1 by ELISA.

The up-regulation of chemokines by cationic peptides was confirmed in several different systems. The murine MCP-1, a homologue of the human MCP-1, is a member of the β(C-C) chemokine family. MCP-1 has been demonstrated to recruit monocytes, NK cells and some T lymphocytes. When RAW 264.7 macrophage cells and whole human blood from 3 donors were stimulated with increasing concentrations of peptide, SEQ ID NO: 1, they produced significant levels of MCP-1 in their supernatant, as judged by ELISA (Table 36). RAW 264.7 cells stimulated with peptide concentrations ranging from 20-50 μg/ml for 24 hr produced significant levels of MCP-1(200-400 pg/ml above background). When the cells (24 h) and whole blood (4 h) were stimulated with 100 μg/ml of SEQ ID NO: 1, high levels of MCP-1 were produced.

The effect of cationic peptides on chemokine induction was also examined in a completely different cell system, A549 human epithelial cells. Interestingly, although these cells produce MCP-1 in response to LPS, and this response could be antagonized by peptide; there was no production of MCP-1 by A549 cells in direct response to peptide, SEQ ID NO: 1. Peptide SEQ ID NO: 1 at high concentrations, did however induce production of IL-8, a neutrophil specific chemokine (Table 37). Thus, SEQ ID NO: 1 can induce a different spectrum of responses from different cell types and at different concentrations. A number of peptides from each of the formula groups were tested for their ability to induce IL-8 in A549 cells (Table 38). Many of these peptides at a low concentration, 10 μg/ml induced IL-8 above background levels. At high concentrations (100 μg/ml) SEQ ID NO: 13 was also found to induce IL-8 in whole human blood (Table 39). Peptide SEQ ID NO: 2 also significantly induced IL-8 in HBE cells (Table 40) and undifferentiated THP-1 cells (Table 41).

BALB/c mice were given SEQ ID NO: 1 or endotoxin-free water by intratracheal instillation and the levels of MCP-1 and TNF-α examined in the bronchioalveolar lavage fluid after 3-4 hr. It was found that the mice treated with 50 μg/ml peptide, SEQ ID NO: 1 produced significantly increased levels of MCP-1 over mice given water or anesthetic alone (Table 42). This was not a pro-inflammatory response to peptide, SEQ ID NO: 1 since peptide did not significantly induce more TNF-α than mice given water or anesthetic alone. peptide, SEQ ID NO: 1 was also found not to significantly induce TNF-α production by RAW 264.7 cells and bone marrow-derived macrophages treated with peptide, SEQ ID NO: 1 (up to 100 μg/ml) (Table 43). Thus, peptide, SEQ ID NO: 1 selectively induces the production of chemokines without inducing the production of inflammatory mediators such as TNF-α. This illustrates the dual role of peptide, SEQ ID NO: 1 as a factor that can block bacterial product-induced inflammation while helping to recruit phagocytes that can clear infections.

TABLE 38
Induction of MCP-1 in RAW 264.7 cells and whole human blood.

| | |
|---|---|
| | Monocyte chemoattractant |
| Peptide, SEQ ID NO: 1 | protein (MCP)-1 (pg/ml)* |

| (μg/ml) | RAW cells | Whole blood |
|---|---|---|
| 0 | 135.3 ± 16.3 | 112.7 ± 43.3 |
| 10 | 165.7 ± 18.2 | 239.3 ± 113.3 |
| 50 | 367 ± 11.5 | 371 ± 105 |
| 100 | 571 ± 17.4 | 596 ± 248.1 |

RAW 264.7 mouse macrophage cells or whole human blood were stimulated with increasing concentrations of SEQ ID NO: 1 for 4 hr. The human blood samples were centrifuged and the serum was removed and tested for MCP-1 by ELISA along with the supernatants from the RAW 264.7 cells. The RAW cell data presented is the mean of three or more experiments ± standard error and the human blood data represents the mean ± standard error from three separate donors.

TABLE 39
Induction of IL-8 in A549 cells and whole human blood.

| | |
|---|---|
| Peptide, SEQ ID NO: 1 | IL-8 (pg/ml) |

| (μg/ml) | A549 cells | Whole blood |
|---|---|---|
| 0 | 172 ± 29.1 | 660.7 ± 126.6 |
| 1 | 206.7 ± 46.1 | |
| 10 | 283.3 ± 28.4 | 945.3 ± 279.9 |
| 20 | 392 ± 31.7 | |
| 50 | 542.3 ± 66.2 | 1160.3 ± 192.4 |
| 100 | 1175.3 ± 188.3 | |

A549 cells or whole human blood were stimulated with increasing concentrations of peptide for 24 and 4 hr respectively. The human blood samples were centrifuged and the serum was removed and tested for IL-8 by ELISA along with the supernatants from the A549 cells. The A549 cell data presented is the mean of three or more experiments ± standard error and the human blood data represents the mean ± standard error from three separate donors.

TABLE 40
Induction of IL-8 in A549 cells by Cationic peptides.

| Peptide (10 ug/ml) | IL-8 (ng/ml) |
|---|---|
| No peptide | 0.164 |
| LPS, no peptide | 0.26 |
| SEQ ID NO: 1 | 0.278 |
| SEQ ID NO: 6 | 0.181 |
| SEQ ID NO: 7 | 0.161 |
| SEQ ID NO: 9 | 0.21 |
| SEQ ID NO: 10 | 0.297 |
| SEQ ID NO: 13 | 0.293 |
| SEQ ID NO: 14 | 0.148 |
| SEQ ID NO: 16 | 0.236 |
| SEQ ID NO: 17 | 0.15 |
| SEQ ID NO: 19 | 0.161 |
| SEQ ID NO: 20 | 0.151 |
| SEQ ID NO: 21 | 0.275 |
| SEQ ID NO: 22 | 0.314 |
| SEQ ID NO: 23 | 0.284 |
| SEQ ID NO: 24 | 0.139 |
| SEQ ID NO: 26 | 0.201 |
| SEQ ID NO: 27 | 0.346 |
| SEQ ID NO: 28 | 0.192 |
| SEQ ID NO: 29 | 0.188 |
| SEQ ID NO: 30 | 0.284 |
| SEQ ID NO: 31 | 0.168 |
| SEQ ID NO: 33 | 0.328 |
| SEQ ID NO: 34 | 0.315 |
| SEQ ID NO: 35 | 0.301 |
| SEQ ID NO: 36 | 0.166 |
| SEQ ID NO: 37 | 0.269 |
| SEQ ID NO: 38 | 0.171 |
| SEQ ID NO: 40 | 0.478 |
| SEQ ID NO: 41 | 0.371 |
| SEQ ID NO: 42 | 0.422 |
| SEQ ID NO: 43 | 0.552 |
| SEQ ID NO: 44 | 0.265 |
| SEQ ID NO: 45 | 0.266 |
| SEQ ID NO: 47 | 0.383 |
| SEQ ID NO: 48 | 0.262 |
| SEQ ID NO: 49 | 0.301 |
| SEQ ID NO: 50 | 0.141 |
| SEQ ID NO: 51 | 0.255 |
| SEQ ID NO: 52 | 0.207 |
| SEQ ID NO: 53 | 0.377 |
| SEQ ID NO: 54 | 0.133 |

A549 human epithelial cells were stimulated with 10 μg of peptide for 24 hr. The supernatant was removed and tested for IL-8 by ELISA.

TABLE 41
Induction by Peptide of IL-8 in human blood.

| SEQ ID NO: 3 (μg/ml) | IL-8 (pg/ml) |
|---|---|
| 0 | 85 |
| 10 | 70 |
| 100 | 323 |

Whole human blood was stimulated with increasing concentrations of peptide for 4 hr. The human blood samples were centrifuged and the serum was removed and tested for IL-8 by ELISA. The data shown is the average 2 donors.

TABLE 42
Induction of IL-8 in HBE cells.

| SEQ ID NO: 2 | |
|---|---|
| (μg/ml) | IL-8 (pg/ml) |
| 0 | 552 ± 90 |
| 0.1 | 670 ± 155 |
| 1 | 712 ± 205 |
| 10 | 941 ± 15 |
| 50 | 1490 ± 715 |

Increasing concentrations of the peptide were incubated with HBE cells for 8 h, the supernantant removed and tested for IL-8. The data is presented as the mean of three or more experiments ± standard error.

TABLE 43
Induction of IL-8 in undifferentiated THP-1 cells.

| SEQ ID NO: 3 | |
|---|---|
| (μg/ml) | IL-8 (pg/ml) |
| 0 | 10.6 |
| 10 | 17.2 |
| 50 | 123.7 |

The human monocyte THP-1 cells were incubated with indicated concentrations of peptide for 8 hr. The supernatant was removed and tested for IL-8 by ELISA.

TABLE 44
Induction of MCP-1 by Peptide, SEQ ID NO: 1 in mouse airway.

| Condition | MCP-1 (pg/ml) | TNF-α (pg/ml) |
|---|---|---|
| Water | 16.5 ± 5 | 664 ± 107 |
| peptide | 111 ± 30 | 734 ± 210 |
| Avertin | 6.5 ± 0.5 | 393 ± 129 |

BALB/c mice were anaesthetised with avertin and given intratracheal instillation of peptide or water or no instillation (no treatment). The mice were monitored for 4 hours, anaesthetised and the BAL fluid was isolated and analyzed for MCP-1 and TNF-α concentrations by ELISA. The data shown is the mean of 4 or 5 mice for each condition ± standard error.

TABLE 45
Lack of Significant TNF-α induction by the Cationic Peptides.

| Peptide Treatment | TNF-α (pg/ml) |
|---|---|
| Media background | 56 ± 8 |
| LPS treatment, No peptide | 15207 ± 186 |
| SEQ ID NO: 1 | 274 ± 15 |
| SEQ ID NO: 5 | 223 ± 45 |
| SEQ ID NO: 6 | 297 ± 32 |
| SEQ ID NO: 7 | 270 ± 42 |
| SEQ ID NO: 8 | 166 ± 23 |
| SEQ ID NO: 9 | 171 ± 33 |
| SEQ ID NO: 10 | 288 ± 30 |
| SEQ ID NO: 12 | 299 ± 65 |
| SEQ ID NO: 13 | 216 ± 42 |
| SEQ ID NO: 14 | 226 ± 41 |
| SEQ ID NO: 15 | 346 ± 41 |
| SEQ ID NO: 16 | 341 ± 68 |
| SEQ ID NO: 17 | 249 ± 49 |
| SEQ ID NO: 19 | 397 ± 86 |
| SEQ ID NO: 20 | 285 ± 56 |
| SEQ ID NO: 21 | 263 ± 8 |
| SEQ ID NO: 22 | 195 ± 42 |
| SEQ ID NO: 23 | 254 ± 58 |
| SEQ ID NO: 24 | 231 ± 32 |
| SEQ ID NO: 26 | 281 ± 34 |
| SEQ ID NO: 27 | 203 ± 42 |
| SEQ ID NO: 28 | 192 ± 26 |
| SEQ ID NO: 29 | 242 ± 40 |
| SEQ ID NO: 31 | 307 ± 71 |
| SEQ ID NO: 33 | 196 ± 42 |
| SEQ ID NO: 34 | 204 ± 51 |
| SEQ ID NO: 35 | 274 ± 76 |
| SEQ ID NO: 37 | 323 ± 41 |
| SEQ ID NO: 38 | 199 ± 38 |
| SEQ ID NO: 43 | 947 ± 197 |
| SEQ ID NO: 44 | 441 ± 145 |
| SEQ ID NO: 45 | 398 ± 90 |
| SEQ ID NO: 48 | 253 ± 33 |
| SEQ ID NO: 49 | 324 ± 38 |
| SEQ ID NO: 50 | 311 ± 144 |
| SEQ ID NO: 53 | 263 ± 40 |
| SEQ ID NO: 54 | 346 ± 86 |

RAW 264.7 macrophage cells were incubated with indicated peptides (40 μg/ml) for 6 hours. The supernatant was collected and tested for levels of TNF-α by ELISA. The data is presented as the mean of three or more experiments + standard error.

### EXAMPLE 6

### Cationic Peptides Increase Surface Expression of Chemokine Receptors

To analyze cell surface expression of IL-8RB, CXCR-4, CCR2, and LFA-1, RAW macrophage cells were stained with 10 μg/ml of the appropriate primary antibody (Santa Cruz Biotechnology) followed by FITC-conjugated goat anti-rabbit IgG [IL-8RB and CXCR-4 (Jackson ImmunoResearch Laboratories, West Grove, Pa.)] or FITC-conjugated donkey anti-goat IgG (Santa Cruz). The cells were analyzed using a FACscan, counting 10,000 events and gating on forward and side scatter to exclude cell debris.

The polynucleotide array data suggested that some peptides up-regulate the expression of the chemokine receptors IL-8RB, CXCR-4 and CCR2 by 10, 4 and 1.4 fold above unstimulated cells respectively. To confirm the polynucleotide array data, the surface expression was examined by flow cytometry of these receptors on RAW cells stimulated with peptide for 4 hr. When 50 μg/ml of peptide was incubated with RAW cells for 4 hr, IL-8RB was upregulated an average of 2.4-fold above unstimulated cells, CXCR-4 was up-regulated an average of 1.6-fold above unstimulated cells and CCR2 was up-regulated 1.8-fold above unstimulated cells (Table 46). As a control CEMA was demonstrated to cause similar up-regulation. SEQ ID NO: 3 was the only peptide to show significant up-regulation of LFA-1 (3.8 fold higher than control cells).

TABLE 46
Increased surface expression of CXCR-4, IL-8RB and
CCR2 in response to peptides.

| | Concentration | Fold Increase in Protein Expression | | |
|---|---|---|---|---|
| Peptide | (μg/ml) | IL-8RB | CXCR-4 | CCR2 |
| SEQ ID NO: 1 | 10 | 1.0 | 1.0 | 1.0 |
| SEQ ID NO: 1 | 50 | 1.3 ± 0.05 | 1.3 ± 0.03 | 1.3 ± 0.03 |
| SEQ ID NO: 1 | 100 | 2.4 ± 0.6 | 1.6 ± 0.23 | 1.8 ± 0.15 |
| SEQ ID NO: 3 | 100 | 2.0 ± 0.6 | Not Done | 4.5 |
| CEMA | 50 | 1.6 ± 0.1 | 1.5 ± 0.2 | 1.5 ± 0.15 |
| CEMA | 100 | 3.6 ± 0.8 | Not Done | 4.7 ± 1.1 |

RAW macrophage cells were stimulated with peptide for 4 hr. The cells were washed and stained with the appropriate primary and FITC-labeled secondary antibodies. The data shown represents the average (fold change of RAW cells stimulated with peptide from media) ± standard error.

### EXAMPLE 7

### Phosphorylation of Map Kinases by Cationic Peptides

The cells were seeded at 2.5×10⁵−5×10⁵ cells/ml and left overnight. They were washed once in media, serum starved in the morning (serum free media—4 hrs). The media was removed and replaced with PBS, then sat at 37° C. for 15 minutes and then brought to room temp for 15 minutes. Peptide was added (concentrations 0.1 μg/ml−50 μg/ml) or H₂O and incubated 10 min. The PBS was very quickly removed and replaced with ice-cold radioimmunoprecipitation (RIPA) buffer with inhibitors (NaF, B-glycerophosphate, MOL, Vanadate, PMSF, Leupeptin Aprotinin). The plates were shaken on ice for 10-15 min or until the cells were lysed and the lysates collected. The procedure for THP-1 cells was slightly different; more cells (2×10⁶) were used. They were serum starved overnight, and to stop the reaction 1 ml of ice-cold PBS was added then they sat on ice 5-10 min, were spun down then resuspended in RIPA. Protein concentrations were determined using a protein assay (Pierce, Rockford, Ill.). Cell lysates (20 μg of protein) were separated by SDS-PAGE and transferred to nitrocellulose filters. The filters were blocked for 1 h with 10 mM Tris-HCI, pH 7.5, 150 mM NaCl (TBS)/5% skim milk powder and then incubated overnight in the cold with primary antibody in TBS/0.05% Tween 20. After washing for 30 min with TBS/0.05% Tween 20, the filters were incubated for 1 h at room temperature with 1 μg/ml secondary antibody in TBS. The filters were washed for 30 min with TBS/0.05% Tween 20 and then incubated 1 h at room temperature with horseradish peroxidase-conjugated sheep anti-mouse IgG (1:10,000 in TBS/0.05% Tween 20). After washing the filters for 30 min with TBS/0.1% Tween 20, immunoreactive bands were visualized by enhanced chemiluminescence (ECL) detection. For experiments with peripheral blood mononuclear cells: The peripheral blood (50-100 ml) was collected from all subjects. Mononuclear cells were isolated from the peripheral blood by density gradient centrifugation on Ficoll-Hypaque. Interphase cells (mononuclear cells) were recovered, washed and then resuspended in recommended primary medium for cell culture (RPMI-1640) with 10% fetal calf serum (FCS) and 1% L-glutamine. Cells were added to 6 well culture plates at 4×10⁶ cells/well and were allowed to adhere at 37° C. in 5% CO₂ atmosphere for 1 hour. The supernatant medium and non-adherent cells were washed off and the appropriate media with peptide was added. The freshly harvested cells were consistently >99% viable as assessed by their ability to exclude trypan blue. After stimulation with peptide, lysates were collected by lysing the cells in RIPA buffer in the presence of various phosphatase- and kinase-inhibitors. Protein content was analyzed and approximately 30 μg of each sample was loaded in a 12% SDS-PAGE gel. The gels were blotted onto nitrocellulose, blocked for 1 hour with 5% skim milk powder in Tris buffered saline (TBS) with 1% TritonX100. Phosphorylation was detected with phosphorylation-specific antibodies.

The results of peptide-induced phosphorylation are summarized in Table 46. SEQ ID NO: 2 was found to cause dose dependent phosphorylation of p38 and ERK1/2 in the mouse macrophage RAW cell line and the HBE cells. SEQ ID NO: 3 caused phosphorylation of MAP kinases in THP-1 human monocyte cell line and phosphorylation of ERK1/2 in the mouse RAW cell line.

TABLE 47
Phosphorylation of MAP kinases in response to peptides.

MAP kinase
phosphorylated

| Cell Line | Peptide | p38 | ERK1/2 |
|---|---|---|---|
| RAW 264.7 | SEQ ID NO: 3 | − | + |
| | SEQ ID NO: 2 | + | + |
| HBE | SEQ ID NO: 3 | | + |
| | SEQ ID NO: 2 | + | + |
| THP-1 | SEQ ID NO: 3 | + | + |
| | SEQ ID NO: 2 | | |

TABLE 48
Peptide Phosphorylation of MAP kinases in human blood monocytes.
SEQ ID NO: 1 at 50 μg/ml) was used to promote phosphorylation.

| | |
|---|---|
| p38 phosphorylation | ERK1/2 phosphorylation |

| 15 minutes | 60 minutes | 15 minutes | 60 minutes |
|---|---|---|---|
| + | − | + | + |

### EXAMPLE 8

### Cationic Peptides Protect Against Bacterial Infection by Enhancing the Immune Response

BALB/c mice were given 1×10⁵ Salmonella and cationic peptide (200 μg) by intraperitoneal injection. The mice were monitored for 24 hours at which point they were euthanized, the spleen removed, homogenized and resuspended in PBS and plated on Luria Broth agar plates with Kanamycin (50 μg/ml). The plates were incubated overnight at 37° C. and counted for viable bacteria (Table 49 and 50). CD-1 mice were given 1×10⁸ S. aureus in 5% porcine mucin and cationic peptide (200 μg) by intraperitoneal injection (Table 51). The mice were monitored for 3 days at which point they were euthanized, blood removed and plated for viable counts. CD-1 male mice were given 5.8×10⁶ CFU EHEC bacteria and cationic peptide (200 μg) by intraperitoneal (IP) injection and monitored for 3 days (Table 52). In each of these animal models a subset of the peptides demonstrated protection against infections. The most protective peptides in the Salmonella model demonstrated an ability to induce a common subset of genes in epithelial cells (Table 53) when comparing the protection assay results in Tables 50 and 51 to the gene expression results in Tables 31-37. This clearly indicates that there is a pattern of gene expression that is consistent with the ability of a peptide to demonstrate protection. Many of the cationic peptides were shown not to be directly antimicrobial as tested by the Minimum Inhibitory Concentration (MIC) assay (Table 54). This demonstrates that the ability of peptides to protect against infection relies on the ability of the peptide to stimulate host innate immunity rather than on direct antimicrobial activity.

TABLE 49
Effect of Cationic Peptides on Salmonella Infection in BALB/c mice.

| Peptide | Viable Bacteria in the Spleen | Statistical Significance |
|---|---|---|
| Treatment | (CFU/ml) | (p value) |
| Control | 2.70 ± 0.84 × 10⁵ | |
| SEQ ID NO: 1 | 1.50 ± 0.26 × 10⁵ | 0.12 |
| SEQ ID NO: 6 | 2.57 ± 0.72 × 10⁴ | 0.03 |
| SEQ ID NO: 13 | 3.80 ± 0.97 × 10⁴ | 0.04 |
| SEQ ID NO: 17 | 4.79 ± 1.27 × 10⁴ | 0.04 |
| SEQ ID NO: 27 | 1.01 ± 0.26 × 10⁵ | 0.06 |

The BALB/c mice were injected IP with Salmonella and Peptide, and 24 h later the animals were euthanized, the spleen removed, homogenized, diluted in PBS and plate counts were done to determine bacteria viability.

TABLE 50
Effect of Cationic Peptides on Salmonella Infection in BALB/c mice.

| Peptide Treatment | Viable Bacteria in the Spleen (CFU/ml) |
|---|---|
| Control | 1.88 ± 0.16 × 10⁴ |
| SEQ ID NO: 48 | 1.98 ± 0.18 × 10⁴ |
| SEQ ID NO: 26 | 7.1 ± 1.37 × 10⁴ |
| SEQ ID NO: 30 | 5.79 ± 0.43 × 10³ |
| SEQ ID NO: 37 | 1.57 ± 0.44 × 10⁴ |
| SEQ ID NO: 5 | 2.75 ± 0.59 × 10⁴ |
| SEQ ID NO: 7 | 5.4 ± 0.28 × 10³ |
| SEQ ID NO: 9 | 1.23 ± 0.87 × 10⁴ |
| SEQ ID NO: 14 | 2.11 ± 0.23 × 10³ |
| SEQ ID NO: 20 | 2.78 ± 0.22 × 10⁴ |
| SEQ ID NO: 23 | 6.16 ± 0.32 × 10⁴ |

The BALB/c mice were injected intraperitoneally with Salmonella and Peptide, and 24 h later the animals were euthanized, the spleen removed, homogenized, diluted in PBS and plate counts were done to determine bacteria viability.

TABLE 51
Effect of Cationic Peptides in a Murine S. aureus infection model.

| | | # Mice Survived (3 days)/Total |
|---|---|---|
| Treatment | CFU/ml (blood) | mice in group |
| No Peptide | 7.61 ± 1.7 × 10³ | 6/8 |
| SEQ ID NO: 1 | 0 | 4/4 |
| SEQ ID NO: 27 | 2.25 ± 0.1 × 10² | 3/4 |
| SEQ ID NO: 30 | 1.29 ± 0.04 × 10² | 4/4 |
| SEQ ID NO: 37 | 9.65 ± 0.41 × 10² | 4/4 |
| SEQ ID NO: 5 | 3.28 ± 1.7 × 10³ | 4/4 |
| SEQ ID NO: 6 | 1.98 ± 0.05 × 10² | 3/4 |
| SEQ ID NO: 7 | 3.8 ± 0.24 × 10³ | 4/4 |
| SEQ ID NO: 9 | 2.97 ± 0.25 × 10² | 4/4 |
| SEQ ID NO: 13 | 4.83 ± 0.92 × 10³ | 3/4 |
| SEQ ID NO: 17 | 9.6 ± 0.41 × 10² | 4/4 |
| SEQ ID NO: 20 | 3.41 ± 1.6 × 10³ | 4/4 |
| SEQ ID NO: 23 | 4.39 ± 2.0 × 10³ | 4/4 |

CD-1 mice were given 1 × 10⁸ bacteria in 5% porcine mucin via intraperitoneal (IP) injection. Cationic peptide (200 μg) was given via a separate IP injection. The mice were monitored for 3 days at which point they were euthanized, blood removed and plated for viable counts. The following peptides were not effective in controlling S. aureus infection: SEQ ID NO: 48, SEQ ID NO: 26.

TABLE 52
Effect of Peptide in a Murine EHEC infection model.

| Treatment | Peptide | Survival (%) |
|---|---|---|
| control | none | 25 |
| SEQ ID NO: 23 | 200 μg | 100 |

CD-1 male mice (5 weeks old) were given 5.8 × 10⁶ CFU EHEC bacteria via intraperitoneal (IP) injection. Cationic peptide (200 μg) was given via a separate IP injection. The mice were monitored for 3 days.

TABLE 53
Up-regulation of patterns of gene expression in A549 epithelial cells induced by
peptides that are active in vivo.

| | | Fold Up regulation of Gene Expression | | | |
|---|---|---|---|---|---|
| | | relative to Untreated Cells | | | |
| | Unstimulated | SEQ ID | SEQ ID | SEQ ID | SEQ ID |
| Target (Accession number) | Cell Intensity | NO: 30 | NO: 7 | NO: 13 | NO: 37 |
| Zinc finger protein (AF061261) | 13 | 2.6 | 9.4 | 9.4 | 1.0 |
| Cell cycle gene (S70622) | 1.62 | 8.5 | 3.2 | 3.2 | 0.7 |
| IL-10 Receptor (U00672) | 0.2 | 2.6 | 9 | 4.3 | 0.5 |
| Transferase (AF038664) | 0.09 | 12.3 | 9.7 | 9.7 | 0.1 |
| Homeobox protein (AC004774) | 0.38 | 3.2 | 2.5 | 2.5 | 1.7 |
| Forkhead protein (AF042832) | 0.17 | 14.1 | 3.5 | 3.5 | 0.9 |
| Unknown (AL096803) | 0.12 | 4.8 | 4.3 | 4.3 | 0.6 |
| KIAA0284 Protein (AB006622) | 0.47 | 3.4 | 2.1 | 2.1 | 1.3 |
| Hypothetical Protein (AL022393) | 0.12 | 4.4 | 4.0 | 4.0 | 0.4 |
| Receptor (AF112461) | 0.16 | 2.4 | 10.0 | 10.0 | 1.9 |
| Hypothetical Protein (AK002104) | 0.51 | 4.7 | 2.6 | 2.6 | 1.0 |
| Protein (AL050261) | 0.26 | 3.3 | 2.8 | 2.8 | 1.0 |
| Polypeptide (AF105424) | 0.26 | 2.5 | 5.3 | 5.3 | 1.0 |
| SPR1 protein (AB031480) | 0.73 | 3.0 | 2.7 | 2.7 | 1.3 |
| Dehydrogenase (D17793) | 4.38 | 2.3 | 2.2 | 2.2 | 0.9 |
| Transferase (M63509) | 0.55 | 2.7 | 2.1 | 2.1 | 1.0 |
| Peroxisome factor (AB013818) | 0.37 | 3.4 | 2.9 | 2.9 | 1.4 |

The peptides SEQ ID NO: 30, SEQ ID NO: 7 and SEQ ID NO: 13 at concentrations of 50 μg/ml were each shown to increase the expression of a pattern of genes after 4 h treatment.
Peptide was incubated with the human A549 epithelial cells for 4 h and the RNA was isolated, converted into labelled cDNA probes and hybridised to Human Operon arrays (PRHU04).
The intensity of polynucleotides in control, unstimulated cells are shown in the second columns for labelling of cDNA (average of Cy3 and Cy5).
The Fold Up regulation column refers to the intensity of polynucleotide expression in peptide-simulated cells divided by the intensity of unstimulated cells.
The SEQ ID NO: 37 peptide was included as a negative control that was not active in the murine infection models.

TABLE 54

| | MIC (μg/ml) | | | | | |
|---|---|---|---|---|---|---|
| Peptide | E. coli | S. aureus | P. aerug. | S. typhim. | C. rhod. | EHEC |
| Polymyxin | 0.25 | 16 | 0.25 | 0.5 | 0.25 | 0.5 |
| Gentamicin | 0.25 | 0.25 | 0.25 | 0.25 | 0.25 | 0.5 |
| SEQ ID NO: 1 | 32 | > | 96 | 64 | 8 | 4 |
| SEQ ID NO: 5 | 128 | > | > | > | 64 | 64 |
| SEQ ID NO: 6 | 128 | > | > | 128 | 64 | 64 |
| SEQ ID NO: 7 | > | > | > | > | > | > |
| SEQ ID NO: 8 | > | > | > | > | > | > |
| SEQ ID NO: 9 | > | > | > | > | > | > |
| SEQ ID NO: 10 | > | > | > | > | > | 64 |
| SEQ ID NO: 12 | > | > | > | > | > | > |
| SEQ ID NO: 13 | > | > | > | > | > | > |
| SEQ ID NO: 14 | > | > | > | > | > | > |
| SEQ ID NO: 15 | 128 | > | > | > | 128 | 64 |
| SEQ ID NO: 16 | > | > | > | > | > | > |
| SEQ ID NO: 17 | > | > | > | > | > | > |
| SEQ ID NO: 19 | 8 | 16 | 16 | 64 | 4 | 4 |
| SEQ ID NO: 2 | 4 | 16 | 32 | 16 | 64 | |
| SEQ ID NO: 20 | 8 | 8 | 8 | 8 | 16 | 8 |
| SEQ ID NO: 21 | 64 | 64 | 96 | 64 | 32 | 32 |
| SEQ ID NO: 22 | 8 | 12 | 24 | 8 | 4 | 4 |
| SEQ ID NO: 23 | 4 | 8 | 8 | 16 | 4 | 4 |
| SEQ ID NO: 24 | 16 | 16 | 4 | 16 | 16 | 4 |
| SEQ ID NO: 26 | 0.5 | 32 | 64 | 2 | 2 | 0.5 |
| SEQ ID NO: 27 | 8 | 64 | 64 | 16 | 2 | 4 |
| SEQ ID NO: 28 | > | > | > | 64 | 64 | 128 |
| SEQ ID NO: 29 | 2 | > | > | 16 | 32 | 4 |
| SEQ ID NO: 30 | 16 | > | 128 | 16 | 16 | 4 |
| SEQ ID NO: 31 | > | > | 128 | > | > | 64 |
| SEQ ID NO: 33 | 16 | 32 | > | 16 | 64 | 8 |
| SEQ ID NO: 34 | 8 | > | > | 32 | 64 | 8 |
| SEQ ID NO: 35 | 4 | 128 | 64 | 8 | 8 | 4 |
| SEQ ID NO: 36 | 32 | > | > | 32 | 32 | 16 |
| SEQ ID NO: 37 | > | > | > | > | > | > |
| SEQ ID NO: 38 | 0.5 | 32 | 64 | 4 | 8 | 4 |
| SEQ ID NO: 40 | 4 | 32 | 8 | 4 | 4 | 2 |
| SEQ ID NO: 41 | 4 | 64 | 8 | 8 | 2 | 2 |
| SEQ ID NO: 42 | 1.5 | 64 | 4 | 2 | 2 | 1 |
| SEQ ID NO: 43 | 8 | 128 | 16 | 16 | 8 | 4 |
| SEQ ID NO: 44 | 8 | > | 128 | 128 | 64 | 64 |
| SEQ ID NO: 45 | 8 | > | 128 | 128 | 16 | 16 |
| SEQ ID NO: 47 | 4 | > | 16 | 16 | 4 | 4 |
| SEQ ID NO: 48 | 16 | > | 128 | 16 | 1 | 2 |
| SEQ ID NO: 49 | 4 | > | 16 | 8 | 4 | 4 |
| SEQ ID NO: 50 | 8 | > | 16 | 16 | 16 | 8 |
| SEQ ID NO: 51 | 4 | > | 8 | 32 | 4 | 8 |
| SEQ ID NO: 52 | 8 | > | 32 | 8 | 2 | 2 |
| SEQ ID NO: 53 | 4 | > | 8 | 8 | 16 | 8 |
| SEQ ID NO: 54 | 64 | > | 16 | 64 | 16 | 32 |

Most cationic peptides studied here and especially the cationic peptides effective in infection models are not significantly antimicrobial. A dilution series of peptide was incubated with the indicated bacteria overnight in a 96-well plate. The lowest concentration of peptide that killed the bacteria was used as the MIC.
The symbol > indicates the MIC is too large to measure. An MIC of 4 μg/ml or less was considered clinically meaningful activity.
Abbreviations:
E. coli, Escherichia coli;
S. aureus, Staphylococcus aureus;
P. aerug, Pseudomonas aeruginosa;
S. Typhim, Salmonella enteritidis ssp. typhimurium;
C. rhod, Citobacter rhodensis;
EHEC, Enterohaemorrhagic E. coli.

### EXAMPLE 9

### Use of Polynucleotides Induced by Bacterial Signaling Molecules in Diagnostic/Screening

S. typhimurium LPS and E. coli 0111:B4 LPS were purchased from Sigma Chemical Co. (St. Louis, Mo.). LTA (Sigma) from S. aureus, was resuspended in endotoxin free water (Sigma). The Limulus amoebocyte lysate assay (Sigma) was performed on LTA preparations to confirm that lots were not significantly contaminated by endotoxin (i.e. <1 ng/ml, a concentration that did not cause significant cytokine production in the RAW cell assay). The CpG oligodeoxynucleotides were synthesized with an Applied Biosystems Inc., Model 392 DNA/RNA Synthesizer, Mississauga, ON., then purified and resuspended in endotoxin-free water (Sigma). The following sequences were used CpG: 5′-TCATGACGTTCCTGACGTT-3′ (SEQ ID NO: 57) and nonCpG: 5′-TTCAGGACTTTCCTCAGGTT-3′ (SEQ ID NO: 58). The nonCpG oligo was tested for its ability to stimulate production of cytokines and was found to cause no significant production of TNF-α or IL-6 and therefore was considered as a negative control. RNA was isolated from RAW 264.7 cells that had been incubated for 4 h with medium alone, 100 ng/ml S. typhimurium LPS, 1 μg/ml S. aureus LTA, or 1 μM CpG (concentrations that led to optimal induction of tumor necrosis factor (TNF-α) in RAW cells). The RNA was used to polynucleotiderate cDNA probes that were hybridized to Clontech Atlas polynucleotide array filters, as described above. The hybridization of the cDNA probes to each immobilized DNA was visualized by autoradiography and quantified using a phosphorimager. Results from at least 2 to 3 independent experiments are summarized in Tables 55-59. It was found that LPS treatment of RAW 264.7 cells resulted in increased expression of more than 60 polynucleotides including polynucleotides encoding inflammatory proteins such as IL-1β, inducible nitric oxide synthase (iNOS), MIP-1α, MIP-1β, MIP-2α, CD40, and a variety of transcription factors. When the changes in polynucleotide expression induced by LPS, LTA, and CpG DNA were compared, it was found that all three of these bacterial products increased the expression of pro-inflammatory polynucleotides such as iNOS, MIP-1α, MIP-2α, IL-1β, IL-15, TNFR1 and NF-κB to a similar extent (Table 57). Table 57 describes 19 polynucleotides that were up-regulated by the bacterial products to similar extents in that their stimulation ratios differed by less than 1.5 fold between the three bacterial products. There were also several polynucleotides that were down-regulated by LPS, LTA and CpG to a similar extent. It was also found that there were a number of polynucleotides that were differentially regulated in response to the three bacterial products (Table 58), which includes many of these polynucleotides that differed in expression levels by more than 1.5 fold between one or more bacterial products). LTA treatment differentially influenced expression of the largest subset of polynucleotides compared to LPS or CpG, including hyperstimulation of expression of Jun-D, Jun-B, Elk-1 and cyclins G2 and A1. There were only a few polynucleotides whose expression was altered more by LPS or CpG treatment. Polynucleotides that had preferentially increased expression due to LPS treatment compared to LTA or CpG treatment included the cAMP response element DNA-binding protein 1 (CRE-BPI), interferon inducible protein 1 and CACCC Box-binding protein BKLF. Polynucleotides that had preferentially increased expression after CpG treatment compared to LPS or LTA treatment included leukemia inhibitory factor (LIF) and protease nexin 1 (PN-1). These results indicate that although LPS, LTA, and CpG DNA stimulate largely overlapping polynucleotide expression responses, they also exhibit differential abilities to regulate certain subsets of polynucleotides.

The other polynucleotide arrays used are the Human Operon arrays (identification number for the genome is PRHU04-S1), which consist of about 14,000 human oligos spotted in duplicate. Probes were prepared from 5 μg of total RNA and labeled with Cy3 or Cy5 labeled dUTP. In these experiments, A549 epithelial cells were plated in 100 mm tissue culture dishes at 2.5×10⁶ cells/dish, incubated overnight and then stimulated with 100 ng/ml E. coli O111:B4 LPS for 4 h. Total RNA was isolated using RNAqueous (Ambion). DNA contamination was removed with DNA-free kit (Ambion). The probes prepared from total RNA were purified and hybridized to printed glass slides overnight at 42° C. and washed. After washing, the image was. captured using a Perkin Elmer array scanner. The image processing software (Imapolynucleotide 5.0, Marina Del Rey, Calif.) determines the spot mean intensity, median intensities, and background intensities. An “in house” program was used to remove background. The program calculates the bottom 10% intensity for each subgrid and subtracts this for each grid. Analysis was performed with Polynucleotidespring software (Redwood City, Calif.). The intensities for each spot were normalized by taking the median spot intensity value from the population of spot values within a slide and comparing this value to the values of all slides in the experiment. The relative changes seen with cells treated with LPS compared to control cells can be found in the Tables below. A number of previously unreported changes that would be useful in diagnosing infection are described in Table 60.

To confirm and assess the functional significance of these changes, the levels of selected mRNAs and proteins were assessed and quantified by densitometry. Northern blots using a CD14, vimentin, and tristetraprolin-specific probe confirmed similar expression after stimulation with all 3 bacterial products (Table 60). Similarly measurement of the enzymatic activity of nitric oxide synthetase, iNOS, using Griess reagent to assess levels of the inflammatory mediator NO, demonstrated comparable levels of NO produced after 24 h, consistent with the similar up-regulation of iNOS expression (Table 59). Western blot analysis confirmed the preferential stimulation of leukaemia inhibitory factor (LIF, a member of the IL-6 family of cytokines) by CpG (Table 59). Other confirmatory experiments demonstrated that LPS up-regulated the expression of TNF-α and IL-6 as assessed by ELISA, and the up-regulated expression of MIP-2α, and IL-1β mRNA and down-regulation of DP-1 and cyclin D mRNA as assessed by Northern blot analysis. The analysis was expanded to a more clinically relevant ex vivo system, by examining the ability of the bacterial elements to stimulate pro-inflammatory cytokine production in whole human blood. It was found that E. coli LPS, S. typhimurium LPS, and S. aureus LTA all stimulated similar amounts of serum TNF-α, and IL-1β. CpG also stimulated production of these cytokines, albeit to much lower levels, confirming in part the cell line data.

TABLE 55
Polynucleotides Up-regulated by E. coli O111:B4 LPS
in A549 Epithelial Cells.

| | | Control:Media | Ratio: |
|---|---|---|---|
| Accession | | only | LPS/ |
| Number | Gene | Intensity | control |
| D87451 | ring finger protein 10 | 715.8 | 183.7 |
| AF061261 | C3H-type zinc finger protein | 565.9 | 36.7 |
| D17793 | aldo-keto reductase family 1, | 220.1 | 35.9 |
| | member C3 | | |
| M14630 | prothymosin, alpha | 168.2 | 31.3 |
| AL049975 | Unknown | 145.6 | 62.3 |
| L04510 | ADP-ribosylation factor | 139.9 | 213.6 |
| | domain protein 1, 64 kD | | |
| U10991 | G2 protein | 101.7 | 170.3 |
| U39067 | eukaryotic translation | 61.0 | 15.9 |
| | initiation factor 3, subunit 2 | | |
| X03342 | ribosomal protein L32 | 52.6 | 10.5 |
| NM_004850 | Rho-associated, coiled-coil | 48.1 | 11.8 |
| | containing protein kinase 2 | | |
| AK000942 | Unknown | 46.9 | 8.4 |
| AB040057 | serine/threonine protein | 42.1 | 44.3 |
| | kinase MASK | | |
| AB020719 | KIAA0912 protein | 41.8 | 9.4 |
| AB007856 | FEM-1-like death receptor | 41.2 | 15.7 |
| | binding protein | | |
| J02783 | procollagen-proline, 2- | 36.1 | 14.1 |
| | oxoglutarate 4-dioxygenase | | |
| AL137376 | Unknown | 32.5 | 17.3 |
| AL137730 | Unknown | 29.4 | 11.9 |
| D25328 | phosphofructokinase, platelet | 27.3 | 8.5 |
| AF047470 | malate dehydrogenase 2, | 25.2 | 8.2 |
| | NAD | | |
| M86752 | stress-induced- | 22.9 | 5.9 |
| | phosphoprotein 1 | | |
| M90696 | cathepsin S | 19.6 | 6.8 |
| AK001143 | Unknown | 19.1 | 6.4 |
| AF038406 | NADH dehydrogenase | 17.7 | 71.5 |
| AK000315 | hypothetical protein | 17.3 | 17.4 |
| | FLJ20308 | | |
| M54915 | pim-1 oncogene | 16.0 | 11.4 |
| D29011 | proteasome subunit, beta | 15.3 | 41.1 |
| | type, 5 | | |
| AK000237 | membrane protein of | 15.1 | 9.4 |
| | cholinergic synaptic vesicles | | |
| AL034348 | Unknown | 15.1 | 15.8 |
| AL161991 | Unknown | 14.2 | 8.1 |
| AL049250 | Unknown | 12.7 | 5.6 |
| AL050361 | PTD017 protein | 12.6 | 13.0 |
| U74324 | RAB interacting factor | 12.3 | 5.2 |
| M22538 | NADH dehydrogenase | 12.3 | 7.6 |
| D87076 | KIAA0239 protein | 11.6 | 6.5 |
| NM_006327 | translocase of inner | 11.5 | 10.0 |
| | mitochondrial membrane 23 | | |
| | (yeast) homolog | | |
| AK001083 | Unknown | 11.1 | 8.6 |
| AJ001403 | mucin 5, subtype B, | 10.8 | 53.4 |
| | tracheobronchial | | |
| M64788 | RAP1, GTPase activating | 10.7 | 7.6 |
| | protein 1 | | |
| X06614 | retinoic acid receptor, alpha | 10.7 | 5.5 |
| U85611 | calcium and integring binding | 10.3 | 8.1 |
| | protein | | |
| U23942 | cytochrome P450, 51 | 10.1 | 10.2 |
| AL031983 | Unknown | 9.7 | 302.8 |
| NM_007171 | protein-O- | 9.5 | 6.5 |
| | mannosyltransferase 1 | | |
| AK000403 | hypothetical protein | 9.5 | 66.6 |
| | FLJ20396 | | |
| NM_002950 | ribophorin I | 9.3 | 35.7 |
| L05515 | cAMP response element- | 8.9 | 6.2 |
| | binding protein CRE-BPa | | |
| X83368 | phosphoinositide-3-kinase, | 8.7 | 27.1 |
| | catalytic, gamma polypeptide | | |
| M30269 | nidogen (enactin) | 8.7 | 5.5 |
| M91083 | chromosome 11 open reading | 8.2 | 6.6 |
| | frame 13 | | |
| D29833 | salivary proline-rich protein | 7.7 | 5.8 |
| AB024536 | immunoglobulin superfamily | 7.6 | 8.0 |
| | containing leucine-rich repeat | | |
| U39400 | chromosome 11 open reading | 7.4 | 7.3 |
| | frame 4 | | |
| AF028789 | unc119 (C. elegans) homolog | 7.4 | 27.0 |
| NM_003144 | signal sequence receptor, | 7.3 | 5.9 |
| | alpha (translocon-associated | | |
| | protein alpha) | | |
| X52195 | arachidonate 5-lipoxygenase- | 7.3 | 13.1 |
| | activating protein | | |
| U43895 | human growth factor- | 6.9 | 6.9 |
| | regulated tyrosine kinase | | |
| | substrate | | |
| L25876 | cyclin-dependent kinase | 6.7 | 10.3 |
| | inhibitor 3 | | |
| L04490 | NADH dehydrogenase | 6.6 | 11.1 |
| Z18948 | S100 calcium-binding protein | 6.3 | 11.0 |
| D10522 | myristoylated alanine-rich | 6.1 | 5.8 |
| | protein kinase C substrate | | |
| NM_014442 | sialic acid binding Ig-like | 6.1 | 7.6 |
| | lectin 8 | | |
| U81375 | solute carrier family 29 | 6.0 | 6.4 |
| AF041410 | malignancy-associated | 5.9 | 5.3 |
| | protein | | |
| U24077 | killer cell immunoglobulin- | 5.8 | 14.4 |
| | like receptor | | |
| AL137614 | hypothetical protein | 4.8 | 6.8 |
| NM_002406 | mannosyl (alpha-1,3-)- | 4.7 | 5.3 |
| | glycoprotein beta-1,2-N- | | |
| | acetylglucosaminyltransferase | | |
| AB002348 | KIAA0350 protein | 4.7 | 7.6 |
| AF165217 | tropomodulin 4 (muscle) | 4.6 | 12.3 |
| Z14093 | branched chain keto acid | 4.6 | 5.4 |
| | dehydrogenase E1, alpha | | |
| | polypeptide | | |
| U82671 | caltractin | 3.8 | 44.5 |
| AL050136 | Unknown | 3.6 | 5.0 |
| NM_005135 | solute carrier family 12 | 3.6 | 5.0 |
| AK001961 | hypothetical protein | 3.6 | 5.9 |
| | FLJ11099 | | |
| AL034410 | Unknown | 3.2 | 21.3 |
| S74728 | antiquitin 1 | 3.1 | 9.2 |
| AL049714 | ribosomal protein L34 | 3.0 | 19.5 |
| | pseudogene 2 | | |
| NM_014075 | PRO0593 protein | 2.9 | 11.5 |
| AF189279 | phospholipase A2, group IIE | 2.8 | 37.8 |
| J03925 | integrin, alpha M | 2.7 | 9.9 |
| NM_012177 | F-box protein Fbx5 | 2.6 | 26.2 |
| NM_004519 | potassium voltage-gated | 2.6 | 21.1 |
| | channel, KQT-like subfamily, | | |
| | member 3 | | |
| M28825 | CD1A antigen, a polypeptide | 2.6 | 16.8 |
| X16940 | actin, gamma 2, smooth | 2.4 | 11.8 |
| | muscle, enteric | | |
| X03066 | major histocompatibility | 2.2 | 36.5 |
| | complex, class II, DO beta | | |
| AK001237 | hypothetical protein | 2.1 | 18.4 |
| | FLJ10375 | | |
| AB028971 | KIAA1048 protein | 2.0 | 9.4 |
| AL137665 | Unknown | 2.0 | 7.3 |
| E. coli O111:B4 LPS (100 ng/ml) increased the expression of many polynucleotides in A549 cells as studied by polynucleotide microarrays. LPS was incubated with the A549 cells for 4 h and the RNA was isolated. 5 μg total RNA was used to make Cy3/Cy5 labelled cDNA probes and hybridised onto Human Operon arrays (PRHU04). The intensity of unstimulated cells is shown in the second column of Table 55. The “Ratio: LPS/control” column refers to the intensity of polynucleotide | | | |
| # expression in LPS simulated cells divided by in the intensity of unstimulated cells. | | | |

TABLE 56
Polynucleotides Down-regulated by E. coli O111:B4 LPS in A549 Epithelial Cells.

| | | Control:Media | |
|---|---|---|---|
| Accession | | only | Ratio: |
| Number | Gene | Intensity | LPS/control |
| NM_017433 | myosin IIIA | 167.8 | 0.03 |
| X60484 | H4 histone family member E | 36.2 | 0.04 |
| X60483 | H4 histone family member D | 36.9 | 0.05 |
| AF151079 | hypothetical protein | 602.8 | 0.05 |
| M96843 | inhibitor of DNA binding 2, dominant | 30.7 | 0.05 |
| | negative helix-loop-helix protein | | |
| S79854 | deiodinase, iodothyronine, type III | 39.4 | 0.06 |
| AB018266 | matrin 3 | 15.7 | 0.08 |
| M33374 | NADH dehydrogenase | 107.8 | 0.09 |
| AF005220 | Homo sapiens mRNA for NUP98-HOXD13 | 105.2 | 0.09 |
| | fusion protein, partial cds | | |
| Z80783 | H2B histone family, member L | 20.5 | 0.10 |
| Z46261 | H3 histone family, member A | 9.7 | 0.12 |
| Z80780 | H2B histone family, member H | 35.3 | 0.12 |
| U33931 | erythrocyte membrane protein band 7.2 | 18.9 | 0.13 |
| | (stomatin) | | |
| M60750 | H2B histone family, member A | 35.8 | 0.14 |
| Z83738 | H2B histone family, member E | 19.3 | 0.15 |
| Y14690 | collagen, type V, alpha 2 | 7.5 | 0.15 |
| M30938 | X-ray repair complementing defective | 11.3 | 0.16 |
| | repair in Chinese hamster cells 5 | | |
| L36055 | eukaryotic translation initiation factor 4E | 182.5 | 0.16 |
| | binding protein 1 | | |
| Z80779 | H2B histone family, member G | 54.3 | 0.16 |
| AF226869 | 5(3)-deoxyribonucleotidase; RB-associated | 7.1 | 0.18 |
| | KRAB repressor | | |
| D50924 | KIAA0134 gene product | 91.0 | 0.18 |
| AL133415 | vimentin | 78.1 | 0.19 |
| AL050179 | tropomyosin 1 (alpha) | 41.6 | 0.19 |
| AJ005579 | RD element | 5.4 | 0.19 |
| M80899 | AHNAK nucleoprotein | 11.6 | 0.19 |
| NM_004873 | BCL2-associated athanogene 5 | 6.2 | 0.19 |
| X57138 | H2A histone family, member N | 58.3 | 0.20 |
| AF081281 | lysophospholipase I | 7.2 | 0.22 |
| U96759 | von Hippel-Lindau binding protein 1 | 6.6 | 0.22 |
| U85977 | Human ribosomal protein L12 pseudogene, | 342.6 | 0.22 |
| | partial cds | | |
| D13315 | glyoxalase I | 7.5 | 0.22 |
| AC003007 | Unknown | 218.2 | 0.22 |
| AB032980 | RU2S | 246.6 | 0.22 |
| U40282 | integrin-linked kinase | 10.1 | 0.22 |
| U81984 | endothelial PAS domain protein 1 | 4.7 | 0.23 |
| X91788 | chloride channel, nucleotide-sensitive, 1A | 9.6 | 0.23 |
| AF018081 | collagen, type XVIII, alpha 1 | 6.9 | 0.24 |
| L31881 | nuclear factor I/X (CCAAT-binding | 13.6 | 0.24 |
| | transcription factor) | | |
| X61123 | B-cell translocation gene 1, anti- | 5.3 | 0.24 |
| | proliferative | | |
| L32976 | mitogen-activated protein kinase kinase | 6.3 | 0.24 |
| | kinase 11 | | |
| M27749 | immunoglobulin lambda-like polypeptide 3 | 5.5 | 0.24 |
| X57128 | H3 histone family, member C | 9.0 | 0.25 |
| X80907 | phosphoinositide-3-kinase, regulatory | 5.8 | 0.25 |
| | subunit, polypeptide 2 | | |
| Z34282 | H. sapiens (MAR11) MUC5AC mRNA for | 100.6 | 0.26 |
| | mucin (partial) | | |
| X00089 | H2A histone family, member M | 4.7 | 0.26 |
| AL035252 | CD39-like 2 | 4.6 | 0.26 |
| X95289 | PERB11 family member in MHC class I | 27.5 | 0.26 |
| | region | | |
| AJ001340 | U3 snoRNP-associated 55-kDa protein | 4.0 | 0.26 |
| NM_014161 | HSPC071 protein | 10.6 | 0.27 |
| U60873 | Unknown | 6.4 | 0.27 |
| X91247 | thioredoxin reductase 1 | 84.4 | 0.27 |
| AK001284 | hypothetical protein FLJ10422 | 4.2 | 0.27 |
| U90840 | synovial sarcoma, X breakpoint 3 | 6.6 | 0.27 |
| X53777 | ribosomal protein L17 | 39.9 | 0.27 |
| AL035067 | Unknown | 10.0 | 0.28 |
| AL117665 | DKFZP586M1824 protein | 3.9 | 0.28 |
| L14561 | ATPase, Ca++ transporting, plasma | 5.3 | 0.28 |
| | membrane 1 | | |
| L19779 | H2A histone family, member 0 | 30.6 | 0.28 |
| AL049782 | Unknown | 285.3 | 0.28 |
| X00734 | tubulin, beta, 5 | 39.7 | 0.29 |
| AK001761 | retinoic acid induced 3 | 23.7 | 0.29 |
| U72661 | ninjurin 1 | 4.4 | 0.29 |
| S48220 | deiodinase, iodothyronine, type I | 1,296.1 | 0.29 |
| AF025304 | EphB2 | 4.5 | 0.30 |
| S82198 | chymotrypsin C | 4.1 | 0.30 |
| Z80782 | H2B histone family, member K | 31.9 | 0.30 |
| X68194 | synaptophysin-like protein | 7.9 | 0.30 |
| AB028869 | Unknown | 4.2 | 0.30 |
| AK000761 | Unknown | 4.3 | 0.30 |
| E. coli O111:B4 LPS (100 ng/ml) decreased the expression of many polynucleotides in A549 cells as studied by polynucleotide microarrays. LPS was incubated with the A549 cells for 4 h and the RNA was isolated. 5 μg total RNA was used to make Cy3/Cy5 labeled cDNA probes and hybridized onto Human Operon arrays (PRHU04). The intensity of unstimulated cells is shown in the second column of the Table. The “Ratio: LPS/control” column refers to the intensity of polynucleotide | | | |
| # expression in LPS simulated cells divided by in the intensity of unstimulated cells. | | | |

TABLE 57
Polynucleotides expressed to similar extents after stimulation by the bacterial
products LPS, LTA, and CpG DNA.

| | Control | | | | |
|---|---|---|---|---|---|
| Accession | Unstim. | Ratio | Ratio | Ratio | |
| number | Intensity | LPS:Control | LTA:Control | CpG:Control | Protein/polynucleotide |
| M15131 | 20 | 82 | 80 | 55 | IL-1β |
| M57422 | 20 | 77 | 64 | 90 | tristetraprolin |
| X53798 | 20 | 73 | 77 | 78 | MIP-2α |
| M35590 | 188 | 50 | 48 | 58 | MIP-1β |
| L28095 | 20 | 49 | 57 | 50 | ICE |
| M87039 | 20 | 37 | 38 | 45 | iNOS |
| X57413 | 20 | 34 | 40 | 28 | TGFβ |
| X15842 | 20 | 20 | 21 | 15 | c-rel proto-oncopolynucleotide |
| X12531 | 489 | 19 | 20 | 26 | MIP-1α |
| U14332 | 20 | 14 | 15 | 12 | IL-15 |
| M59378 | 580 | 10 | 13 | 11 | TNFR1 |
| U37522 | 151 | 6 | 6 | 6 | TRAIL |
| M57999 | 172 | 3.8 | 3.5 | 3.4 | NF-κB |
| U36277 | 402 | 3.2 | 3.5 | 2.7 | I-κB (alpha subunit) |
| X76850 | 194 | 3 | 3.8 | 2.5 | MAPKAP-2 |
| U06924 | 858 | 2.4 | 3 | 3.2 | Stat 1 |
| X14951 | 592 | 2 | 2 | 2 | CD18 |
| X60671 | 543 | 1.9 | 2.4 | 2.8 | NF-2 |
| M34510 | 5970 | 1.6 | 2 | 1.4 | CD14 |
| X51438 | 2702 | 1.3 | 2.2 | 2.0 | vimentin |
| X68932 | 4455 | 0.5 | 0.7 | 0.5 | c-Fms |
| Z21848 | 352 | 0.5 | 0.6 | 0.6 | DNA polymerase |
| X70472 | 614 | 0.4 | 0.6 | 0.5 | B-myb |

Bacterial products (100 ng/ml S. typhimurium LPS, 1 μg/ml S. aureus LTA or 1 μM CpG) were shown to potently induce the expression of several polynucleotides. Peptide was incubated with the RAW cells for 4 h and the RNA was isolated, converted into labeled cDNA probes and hybridized to Atlas arrays.
The intensity of control, unstimulated cells is shown in the second column.
The “Ratio LPS/LTA/CpG: Control” column refers to the intensity of polynucleotide expression in bacterial product-simulated cells divided by the intensity of unstimulated cells.

TABLE 58
Polynucleotides that were differentially regulated by the bacterial products
LPS, LTA, and CpG DNA.

| | Unstim. | | | | |
|---|---|---|---|---|---|
| Accession | Control | Ratio | Ratio | Ratio | |
| number | Intensity | LPS:Contrl | LTA:Contrl | CpG:Contrl | Protein/polynucleotide |
| X72307 | 20 | 1.0 | 23 | 1.0 | hepatocyte growth factor |
| L38847 | 20 | 1.0 | 21 | 1.0 | hepatoma transmembrane kinase |
| | | | | | ligand |
| L34169 | 393 | 0.3 | 3 | 0.5 | thrombopoietin |
| J04113 | 289 | 1 | 4 | 3 | Nur77 |
| Z50013 | 20 | 7 | 21 | 5 | H-ras proto-oncopolynucleotide |
| X84311 | 20 | 4 | 12 | 2 | Cyclin A1 |
| U95826 | 20 | 5 | 14 | 2 | Cyclin G2 |
| X87257 | 123 | 2 | 4 | 1 | Elk-1 |
| J05205 | 20 | 18 | 39 | 20 | Jun-D |
| J03236 | 20 | 11 | 19 | 14 | Jun-B |
| M83649 | 20 | 71 | 80 | 42 | Fas 1 receptor |
| M83312 | 20 | 69 | 91 | 57 | CD40L receptor |
| X52264 | 20 | 17 | 23 | 9 | ICAM-1 |
| M13945 | 573 | 2 | 3 | 2 | Pim-1 |
| U60530 | 193 | 2 | 3 | 3 | Mad related protein |
| D10329 | 570 | 2 | 3 | 2 | CD7 |
| X06381 | 20 | 55 | 59 | 102 | Leukemia inhibitory factor (LIF) |
| X70296 | 20 | 6.9 | 13 | 22 | Protease nexin 1 (PN-1) |
| U36340 | 20 | 38 | 7 | 7 | CACCC Box-binding protein |
| | | | | | BKLF |
| S76657 | 20 | 11 | 6 | 7 | CRE-BPI |
| U19119 | 272 | 10 | 4 | 4 | interferon inducible protein 1 |

Bacterial products (100 ng/ml S. typhimurium LPS, 1 μg/ml S. aureus LTA or 1 μM CpG) were shown to potently induce the expression of several polynucleotides. Peptide was incubated with the RAW cells for 4 h and the RNA was isolated, converted into labeled cDNA probes and hybridized to Atlas arrays.
The intensity of control, unstimulated cells is shown in the second column.
The “Ratio LPS/LTA/CpG: Control” column refers to the intensity of polynucleotide expression in bacterial product-simulated cells divided by the intensity of unstimulated cells.

TABLE 59
Confirmation of Table 57 and 58 Array Data.

| | Relative levels | | | |
|---|---|---|---|---|
| Product | Untreated | LPS | LTA | CpG |
| CD14^{a} | 1.0 | 2.2 ± 0.4 | 1.8 ± 0.2 | 1.5 ± 0.3 |
| Vimentin^{a} | 1.0 | 1.2 ± 0.07 | 1.5 ± 0.05 | 1.3 ± 0.07 |
| Tristetraprolin^{a} | 1.0 | 5.5 ± 0.5 | 5.5 ± 1.5 | 9.5 ± 1.5 |
| LIF^{b} | 1.0 | 2.8 ± 1.2 | 2.7 ± 0.6 | 5.1 ± 1.6 |
| NO^{c} | 8 ± 1.5 | 47 ± 2.5 | 20 ± 3 | 21 ± 1.5 |
| ^{a}Total RNA was isolated from unstimulated RAW macrophage cells and cells treated for 4 hr with 100 ng/ml S. typhimurium LPS, 1 μg/ml S. aureus LTA, 1 μM CpG DNA or media alone and Northern blots were performed the membrane was probed for GAPDH, CD14, vimentin, and tristetraprolin as described previously [Scott et al]. The hybridization | | | | |
| # intensities of the Northern blots were compared to GAPDH to look for inconsistencies in loading. These experiments were repeated at least three times and the data shown is the average relative levels of each condition compared to media (as measured by densitometry) ± standard error. | | | | |
| ^{b}RAW 264.7 cells were stimulated with 100 ng/ml S. typhimurium LPS, 1 μg/ml S. aureus LTA, 1 μM CpG DNA or media alone for 24 hours. Protein lysates were prepared, run on SDS PAGE gels and western blots were performed to detect LIF (R&D Systems). These experiments were repeated at least three times and the data shown is the relative levels of LIF | | | | |
| # compared to media (as measured by densitometry) ± standard error. | | | | |

^{c}Supernatant was collected from RAW macrophage cells treated with 100 ng/ml S. typhimurium LPS, 1 μg/ml S. aureus LTA, 1 μM CpG DNA, or media alone for 24 hours and tested for the amount of NO formed in the supernatant as estimated from the accumulation of the stable NO metabolite nitrite with the Griess reagent as described previously [Scott, et al].
The data shown is the average of three experiments ± standard error.

TABLE 60
Pattern of Gene expression in A549 Human Epithelial cells up-regulated
by bacterial signaling molecules (LPS).

| Accession | |
|---|---|
| Number | Gene |
| AL050337 | interferon gamma receptor 1 |
| U05875 | interferon gamma receptor 2 |
| NM_002310 | leukemia inhibitory factor receptor |
| U92971 | coagulation factor II (thrombin) receptor-like 2 |
| Z29575 | tumor necrosis factor receptor superfamily member 17 |
| L31584 | Chemokine receptor 7 |
| J03925 | cAMP response element-binding protein |
| M64788 | RAP1, GTPase activating protein |
| NM_004850 | Rho-associated kinase 2 |
| D87451 | ring finger protein 10 |
| AL049975 | Unknown |
| U39067 | eukaryotic translation initiation factor 3, subunit 2 |
| AK000942 | Unknown |
| AB040057 | serine/threonine protein kinase MASK |
| AB020719 | KIAA0912 protein |
| AB007856 | FEM-1-like death receptor binding protein |
| AL137376 | Unknown |
| AL137730 | Unknown |
| M90696 | cathepsin S |
| AK001143 | Unknown |
| AF038406 | NADH dehydrogenase |
| AK000315 | hypothetical protein FLJ20308 |
| M54915 | pim-1 oncogene |
| D29011 | proteasome subunit, beta type, 5 |
| AL034348 | Unknown |
| D87076 | KIAA0239 protein |
| AJ001403 | mucin 5, subtype B, tracheobronchial |
| J03925 | integrin, alpha M |
| E. coli O111:B4 LPS (100 ng/ml) increased the expression of many polynucleotides in A549 cells as studied by polynucleotide microarrays. LPS was incubated with the A549 cells for 4 h and the RNA was isolated. 5 μg total RNA was used to make Cy3/Cy5 labelled cDNA probes and hybridised onto Human Operon arrays (PRHU04). The examples of polynucleotide | |
| # expression changes in LPS simulated cells represent a greater than 2-fold intensity level change of LPS treated cells from untreated cells. | |

### EXAMPLE 10

### Altering Signaling to Protect Against Bacterial Infections

The Salmonella Typhimurium strain SL1344 was obtained from the American Type Culture Collection (ATCC; Manassas, Va.) and grown in Luria-Bertani (LB) broth. For macrophage infections, 10 ml LB in a 125 mL flask was inoculated from a frozen glycerol stock and cultured overnight with shaking at 37° C. to stationary phase. RAW 264.7 cells (1×10⁵ cells/well) were seeded in 24 well plates. Bacteria were diluted in culture medium to give a nominal multiplicity of infection (MOI) of approximately 100, bacteria were centrifuged onto the monolayer at 1000 rpm for 10 minutes to synchronize infection, and the infection was allowed to proceed for 20 min in a 37° C., 5% CO₂ incubator. Cells were washed 3 times with PBS to remove extracellular bacteria and then incubated in DMEM+10% FBS containing 100 μg/ml gentamicin (Sigma, St. Louis, Mo.) to kill any remaining extracellular bacteria and prevent re-infection. After 2 h, the gentamicin concentration was lowered to 10 μg/ml and maintained throughout the assay. Cells were pretreated with inhibitors for 30 min prior to infection at the following concentrations: 50 μM PD 98059 (Calbiochem), 50 μM U 0126 (Promega), 2 mM diphenyliodonium (DPI), 250 μM acetovanillone (apocynin, Aldrich), 1 mM ascorbic acid (Sigma), 30 mM N-acetyl cysteine (Sigma), and 2 mM N^{g}-L-monomethyl arginine (L-NMMA, Molecular Probes) or 2 mM N^{G}-D-monomethyl arginine (D-NMMA, Molecular Probes). Fresh inhibitors were added immediately after infection, at 2 h, and 6-8 h post-infection to ensure potency. Control cells were treated with equivalent volumes of dimethylsulfoxide (DMSO) per mL of media. Intracellular survival/replication of S. Typhimurium SL1344 was determined using the gentamicin-resistance assay, as previously described. Briefly, cells were washed twice with PBS to remove gentamicin, lysed with 1% Triton X-100/0.1% SDS in PBS at 2 h and 24 h post-infection, and numbers of intracellular bacteria calculated from colony counts on LB agar plates. Under these infection conditions, macrophages contained an average of 1 bacterium per cell as assessed by standard plate counts, which permitted analysis of macrophages at 24 h post-infection. Bacterial filiamnentation is related to bacterial stress. NADPH oxidase and iNOS can be activated by MEK/ERK signaling. The results (Table 61) clearly demonstrate that the alteration of cell signaling is a method whereby intracellular Salmonella infections can be resolved. Thus since bacteria to up-regulate multiple genes in human cells, this strategy of blocking signaling represents a general method of therapy against infection.

TABLE 61
Effect of the Signaling Molecule MEK on Intracellular Bacteria in IFN-γ-
primed RAW cells.

| Treatment^{a} | Effect^{b} |
|---|---|
| 0 | None |
| MEK inhibitor | Decrease bacterial filamentation (bacterial stress)^{c} |
| U 0126 | Increase in the number of intracellular S. Typhimurium |
| MEK inhibitor | Decrease bacterial filamentation (bacterial stress)^{c} |
| PD 98059 | Increase in the number of intracellular S. Typhimurium |
| NADPH | Decrease bacterial filamentation (bacterial stress)^{c} |
| oxidase inhibitor^{d} | Increase in the number of intracellular S. Typhimurium |

### EXAMPLE 11

### Anti-Viral Activity

SDF-1, a C-X-C chemokine is a natural ligand for HIV-1 coreceptor-CXCR4. The chemokine receptors CXCR4 and CCR5 are considered to be potential targets for the inhibition of HIV-1 replication. The crystal structure of SDF-1 exhibits antiparallel β-sheets and a positively charged surface, features that are critical in binding to the negatively charged extracellular loops of CXCR4. These findings suggest that chemokine derivatives, small-size CXCR4 antagonists, or agonists mimicking the structure or ionic property of chemokines may be useful agents for the treatment of X4 HIV-1 infection. It was found that the cationic peptides inhibited SDF-1 induced T-cell migration suggesting that the peptides may act as CXCR4 antagonists. The migration assays were performed as follows. Human Jurkat T cells were resuspended to 5×10⁶/ml in chemotaxis medium (RPMI 1640/10 mM Hepes/0.5% BSA). Migration assays were performed in 24 well plates using 5 pm polycarbonate Transwell inserts (Costar). Briefly, peptide or controls were diluted in chemotaxis medium and placed in the lower chamber while 0.1 ml cells (5×10⁶/ml) was added to the upper chamber. After 3 hr at 37° C., the number of cells that had migrated into the lower chamber was determined using flow cytometry. The medium from the lower chamber was passed through a FACscan for 30 seconds, gating on forward and side scatter to exclude cell debris. The number of live cells was compared to a “100% migration control” in which 5×10⁵/ml cells had been pipetted directly into the lower chamber and then counted on the FACscan for 30 seconds. The results demonstrate that the addition of peptide results in an inhibition of the migration of Human Jurkat T-cells (Table 62) probably by influencing CXCR4 expression (Tables 63 and 64).

TABLE 62
Peptide inhibits the migration of human Jurkat-T cells:

| | Migration (%) | | | |
|---|---|---|---|---|
| | Positive | SDF-1 | SDF-1 + SEQ 1D | Negative |
| Experiment | control | (100 ng/ml) | 1 (50 μg/ml) | control |
| 1 | 100% | 32% | 0% | <0.01% |
| 2 | 100% | 40% | 0% | 0% |

TABLE 63
Corresponding polynucleotide array data to Table 56:

| Poly- | | | | |
|---|---|---|---|---|
| nucle- | Poly- | | | |
| otide/ | nucleotide | Unstimulated | Ratio | Accession |
| Protein | Function | Intensity | peptide:Unstimulated | Number |
| CXCR- | Chemokine | 36 | 4 | D87747 |
| 4 | receptor | | | |

TABLE 64
Corresponding FACs data to Tables 62 and 63:

| | | Fold Increase in Protein |
|---|---|---|
| | Concentration | Expression |
| Peptide | (μg/ml) | CXCR-4 |
| SEQ ID NO: 1 | 10 | No change |
| SEQ ID NO: 1 | 50 | 1.3 ± 0.03 |
| SEQ ID NO: 1 | 100 | 1.6 ± 0.23 |
| SEQ ID NO: 3 | 100 | 1.5 ± 0.2 |

### EXAMPLE 12

### Synergistic Combinations

Methods and Materials

S. aureus was prepared in phosphate buffered solution (PBS) and 5% porcine mucin (Sigma) to a final expected concentration of 1-4×10⁷ CFU/ml. 100 μl of S. aureus (mixed with 5% porcine, mucin) was injected intraperitoneally (IP) into each CD-1 mouse (6-8 weeks female weighing 20-25 g (Charles River)). Six hours after the onset of infection, 100 μl of the peptide was injected (50-200 μg total) IP along with 0.1 mg/kg Cefepime. After 24 hours, animals were sacrificed and heart puncture was performed to remove 100 μl of blood. The blood was diluted into 1 ml PBS containing Heparin. This was then ftrther diluted and plated for viable colony counts on Mueller-Hinton agar plates (10⁻¹, 10⁻², 10⁻³ & 10⁻⁴). Viable colonies, colony-forming units (CFU), were counted after 24 hours. Each experiment was carried out a minimum of three times. Data is presented as the average CFU+standard error per treatment group (8-10 mice/group).

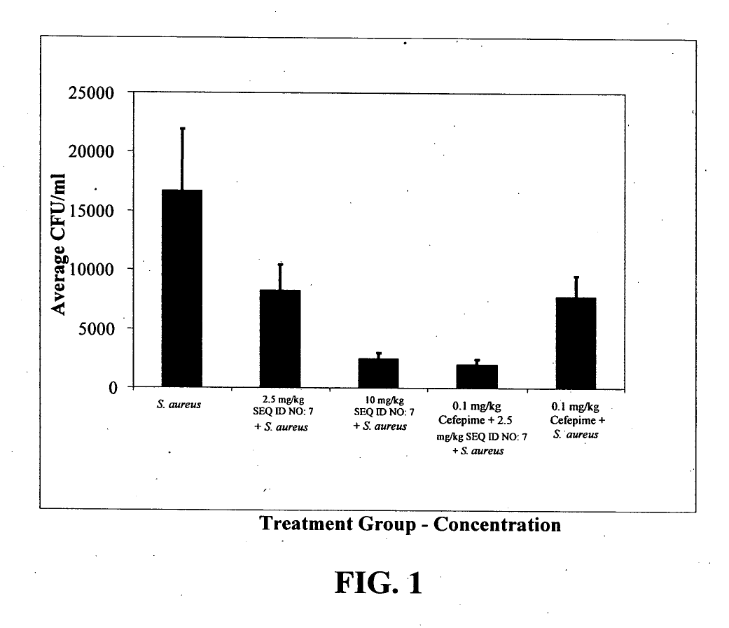

Experiments were carried out with peptide and sub-optimal Cefepime given 6 hours after the onset of systemic S. aureus infection (FIG. 1). The data in FIG. 1 is presented as the mean±standard error of viable counts from blood taken from the mice 24 hrs after the onset of infection. The combination of sub optimal antibiotic (cefepime) dosing and SEQ ID NO: 7 resulted in improved therapeutic efficacy. The ability of the peptides to work in combination with sub-optimal concentrations of an antibiotic in a murine infection model is an important finding. It suggests the potential for extending the life of antibiotics in the clinic and reducing incidence of antibiotic resistance.

SEQ ID NO: 1, as an example, induced phosphorylation and activation of the mitogen activated protein kinases, ERK1/2 and p38 in human peripheral blood-derived monocytes and a human bronchial epithelial cell line but not in B- or T-lymphocytes. Phosphorylation was not dependent on the G-protein coupled receptor, FPRL-1, which was previously proposed to be the receptor for SEQ ID NO: 1-induced chemotaxis on human monocytes and T cells. Activation of ERK1/2 and p38 was markedly increased by the presence of granulocyte macrophage-colony stimulating factor (GM-CSF), but not macrophage-colony stimulating factor (M-CSF). Exposure to SEQ ID NO: 1 also led to the activation of Elk1/2, a transcription factor that is downstream of and activated by phosphorylated ERK1/2, as well as the up-regulation of various Elk-1 controlled genes. The ability of SEQ ID NO: 1 to signal through these pathways has broad implications in immunity, monocyte activation, proliferation and differentiation.

SEQ ID NO: 1 (sequence LGDFFRKSKEKIGKEFKRIVQRIKDFLRNLVPRTES), was synthesized by Fmoc [(N-(9-fluorenyl) methoxycarbonyl)] chemistry at the Nucleic Acid/Protein Synthesis (NAPS) Unit at UBC. Human recombinant granulocyte-macrophage colony-stimulating factor (GM-CSF), interleukin-4 (IL-4) and macrophage colony-stimulating factor (M-CSF) were purchased from Research Diagnostics Inc. (Flanders, N.J., USA). Pertussis toxin was supplied by List Biological Laboratories Inc. (Campbell, Calif., USA).

Blood monocytes were prepared using standard techniques. Briefly, 100 ml of fresh human venous blood was collected in sodium heparin Vacutainer collection tubes (Becton Dickinson, Mississauga, ON, Canada) from volunteers according to UBC Clinical Research Ethics Board protocol C02-0091. The blood was mixed, at a 1:1 ratio, with RPMI 1640 media [supplemented with 10% v/v fetal calf serum (FBS), 1% L-glutamine, 1 nM sodium pyruvate] in an E-toxa-clean (Sigma-Aldrich, Oakville, ON, Canada) washed, endotoxin-free bottle. PBMC were separated using Ficoll-Paque Plus (Amersham Pharmacia Biotech, Baie D'Urfé, PQ, Canada) at room temperature and washed with phosphate buffered saline (PBS). Monocytes were enriched with the removal of T-cells by rosetting with fresh sheep red blood cells (UBC animal care unit) pre-treated with Vibrio cholerae neuraminidase (Calbiochem Biosciences Inc., La Jolla, Calif., USA) and repeat separation by Ficoll Paque Plus. The enriched monocytes were washed with PBS, then cultured (approximately 2-3×10⁶ per well) for 1 hour at 37° C. followed by the removal of non-adherent cells; monocytes were >95% pure as determined by flow cytometry (data not shown). B-lymphocytes were isolated by removing non-adherent cells and adding them to a new plate for one hour at 37° C. This was repeated a total of three times. Any remaining monocytes adhered to the plates, and residual non-adherent cells were primarily B cells. Cells were cultured in Falcon tissue culture 6-well plates (Becton Dickinson, Mississauga, ON, Canada). The adherent monocytes were cultured in 1 ml media at 37° C. in which SEQ ID NO: 1 and/or cytokines dissolved in endotoxin-free water (Sigma-Aldrich, Oakville, ON, Canada) were added. Endotoxin-free water was added as a vehicle control. For studies using pertussis toxin the media was replaced with 1 ml of fresh media containing 100 ng/ml of toxin and incubated for 60 min at 37° C. SEQ ID NO: 1 and cytokines were added directly to the media containing pertussis toxin. For the isolation of T lymphocytes, the rosetted T cells and sheep red blood cells were resuspended in 20 ml PBS and 10 ml of distilled water was added to lyse the latter. The cells were then centrifuged at 1000 rpm for 5 min after which the supernatant was removed. The pelleted T cells were promptly washed in PBS and increasing amounts of water were added until all sheep red blood cells had lysed. The remaining T cells were washed once in PBS, and viability was confirmed using a 0.4% Trypan blue solution. Primary human blood monocytes and T cells were cultured in RPMI 1640 supplemented with 10% v/v heat-inactivated FBS, 1% v/v L-glutamine, 1 nM sodium pyruvate (GIBCO Invitrogen Corporation, Burlington, ON, Canada). For each experiment between two and eight donors were used.

The simian virus 40-transformed, immortalized 16HBE4o-bronchial epithelial cell line was a generous gift of Dr. D. Gruenert (University of California, San Francisco, Calif.). Cells were routinely cultured to confluence in 100% humidity and 5% CO₂ at 37° C. They were grown in Minimal Essential media with Earles' salts (GIBCO Invitrogen Corporation, Burlington, ON, Canada) containing 10% FBS (Hyclone), 2mM L-glutamine. For experiments, cells were grown on Costar Transwell inserts (3-μm pore size, Fischer Scientific) in 24-well plates. Cells were seeded at 5×10⁴ cells per 0.25 ml of media on the top of the inserts while 0.95 ml of media was added to the bottom of the well and cultured at 37° C. and 5% CO₂. Transmembrane resistance was measured daily with a Millipore voltohmeter and inserts were used for experiments typically after 8 to 10 days, when the resistance was 500-700 ohms. The cells were used between passages 8 and 20.

Western Immunoblotting—After stimulation, cells were washed with ice-cold PBS containing 1 mM vanadate (Sigma). Next 125 μl of RIPA buffer (50 mM Tris-HCI, pH 7.4, NP-40 1%, sodium deoxycholate 0.25%, NaCl 150 mM, EDTA 1 mM, PMSF 1 mM, Aprotinin, leupeptin, pepstatin 1 μg/ml each, sodium orthovanadate 1 mM, NaF 1 mM) was added and the cells were incubated on ice until they were completely lysed as assessed by visual inspection. The lysates were quantitated using a BCA assay (Pierce). 30 μg of lysate was loaded onto 1.5 mm thick gels, which were run at 100 volts for approximately 2 hours. Proteins were transferred to nitrocellulose filters for 75 min at 70 V. The filters were blocked for 2 hours at room temperature with 5% skim milk in TBST (10 mM Tris-HCI pH 8, 150 mM NaCl, 0.1% Tween-20). The filters were then incubated overnight at 4° C. with the anti-ERK1/2-P or anti-p38-P (Cell Signaling Technology, Ma) monoclonal antibodies. Immunoreactive bands were detected using horseradish peroxidase-conjugated sheep anti-mouse IgG antibodies (Amersham Pharmacia, New Jersey) and chemiluminescence detection (Sigma, Mo). To quantify bands, the films were scanned and then quantified by densitometry using the software program, ImageJ. The blots were reprobed with β-actin antibody (ICN Biomedical Incorporated, Ohio) and densitometry was performed to allow correction for protein loading.

Kinase Assay—An ERK1/2 activity assay was performed using a non-radioactive kit (Cell Signaling Technology). Briefly, cells were treated for 15 min and lysed in lysis buffer. Equal amounts of proteins were immunoprecipitated with an immobilized phospho-ERK1/2 antibody that reacts only with the phosphorylated (i.e. active) form of ERK1/2. The immobilized precipitated enzymes were then used for the kinase assay using Elk-1 followed by Western blot analysis with antibodies that allow detection and quantitation of phosphorylated substrates.

Quantification of IL-8—Human IL-8 from supernatants of 16HBE40-cells was measured by using the commercially available enzyme-linked immunosorbent assay kit (Biosource) according to the manufacturer's instructions.

Semiquantitative RT-PCR—Total RNA from two independent experiments was isolated from 16HBE4o-cells using RNaqueous (Ambion) as described by the manufacturer. The samples were DNase treated, and then cDNA synthesis was accomplished by using a first-strand cDNA synthesis kit (Gibco). The resultant cDNAs were used as a template in PCRs for various cytokine genes:

(SEQ ID NO: 59)

| | |
|---|---|
| MCP-1 | 5′-TCATAGCAGCCACCTTCATTC-3′; |

(SEQ ID NO: 60)

5′-TAGCGCAGATTCTTGGGTTG-3′;

(SEQ ID NO: 61)

| | |
|---|---|
| MCP-3 | 5′-TGTCCTTTCTCAGAGTGGTTCT-3′; |

(SEQ ID NO: 62)

5′-TGCTTCCATAGGGACATCATA-3′

(SEQ ID NO: 63)

| | |
|---|---|
| IL-6 | 5′-ACCTGAACCTTCCAAAGATGG-3′; |

(SEQ ID NO: 64)

| | |
|---|---|
| | 5′-GCGCAGAATGAGATGAGTTG-3′; |
| and | |

(SEQ ID NO: 65)

| | |
|---|---|
| IL-8 | 5′-GTGCAGAGGGTTGTGGAGAAG-3′; |

(SEQ ID NO: 66)

5′-TTCTCCCGTGCAATATCTAGG-3′

Each RT-PCR reaction was performed in at least duplicate. Results were analysed in the linear phase of amplification and normalized to the housekeeping control, glyceraldehyde-3-phosphate dehydrogenase. Reactions were verified for RNA amplification by including controls without reverse transcriptase.

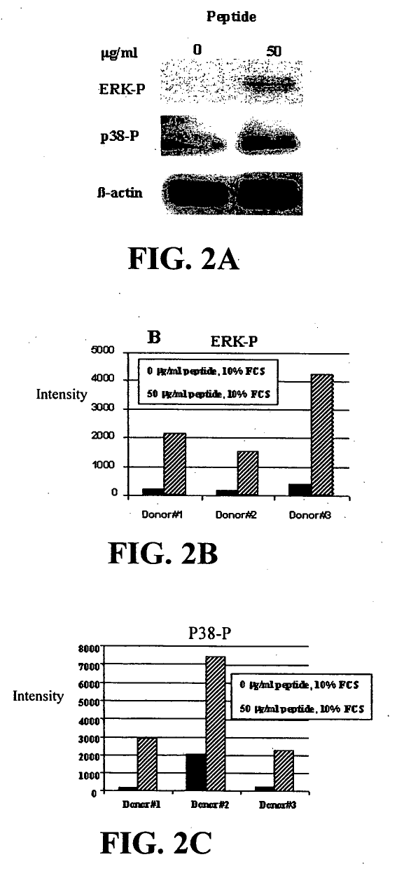

Peptides induce ERK1/2 and p38 phosphorylation in peripheral blood derived monocytes. To determine if peptide induced the activation of the MAP kinases, ERK1/2 and/or p38, peripheral blood derived monocytes were treated with 50 μg/ml SEQ ID NO: 1 or water (as a vehicle control) for 15 min. To visualize the activated (phosphorylated) form of the kinases, Western blots were performed with antibodies specific for the dually phosphorylated form of the kinases (phosphorylation on Thr202+Tyr204 and Thr180+Tyr182 for ERK1/2 and p38 respectively). The gels were re-probed with an antibody for β-actin to normalize for loading differences. In all, an increase in phosphorylation of ERK1/2 (n=8) and p38 (n=4) was observed in response to SEQ ID NO: 1 treatment (FIG. 2).

FIG. 2 shows exposure to SEQ ID NO: 1 induces phosphorylation of ERK1/2 and p38. Lysates from human peripheral blood derived monocytes were exposed to 50 μg/ml of SEQ ID NO: 1 for 15 minutes. A) Antibodies specific for the phosphorylated forms of ERK and p38 were used to detect activation of ERK1/2 and p38. All donors tested showed increased phosphorylation of ERK1/2 and p38 in response to SEQ ID NO: 1 treatment. One representative donor of eight. Relative amounts of phosphorylation of ERK (B) and p38(C) were determined by dividing the intensities of the phosphorylated bands by the intensity of the corresponding control band as described in the Materials and Methods.

Peptide induced activation of ERK1/2 is greater in human serum than in fetal bovine serum. It was demonstrated that SEQ ID NO: 1 induced phosphorylation of ERK1/2 did not occur in the absence of serum and the magnitude of phosphorylation was dependent upon the type of serum present such that activation of ERK1/2 was far superior in human serum (HS) than in fetal bovine serum (FBS).

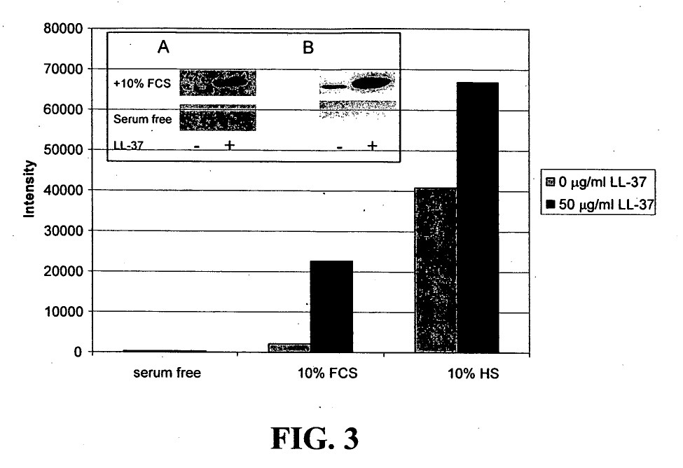

FIG. 3 shows SEQ ID NO: 1 induced phosphorylation of ERK1/2 does not occur in the absence of serum and the magnitude of phosphorylation is dependent upon the type of serum present. Human blood derived monocytes were treated with 50 μg/ml of SEQ ID NO: 1 for 15 minutes. Lysates were run on a 12% acrylamide gel then transferred to nitrocellulose membrane and probed with antibodies specific for the phosphorylated (active) form of the kinase. To normalize for protein loading, the blots were reprobed with β-actin. Quantification was done with ImageJ software.The FIG. 3 inset demonstrates that SEQ ID NO: 1 is unable to induce MAPK activation in human monocytes under serum free conditions. Cells were exposed to 50 mg/ml of SEQ ID NO: 1 (+), or endotoxin free water (−) as a vehicle control, for 15 minutes. (A) After exposure to SEQ ID NO: 1 in media containing 10% fetal calf serun, phosphorylated ERK1/2 was detectable, however, no phosphorylation of ERK1/2 was detected in the absence of serum (n=3). (B) Elk-1, a transcription factor downstream of ERK1/2, was activated (phosphorylated) upon exposure to 50 μg/ml of SEQ ID NO: 1 in media containing 10% fetal calf serum, but not in the absence of serum (n=2).

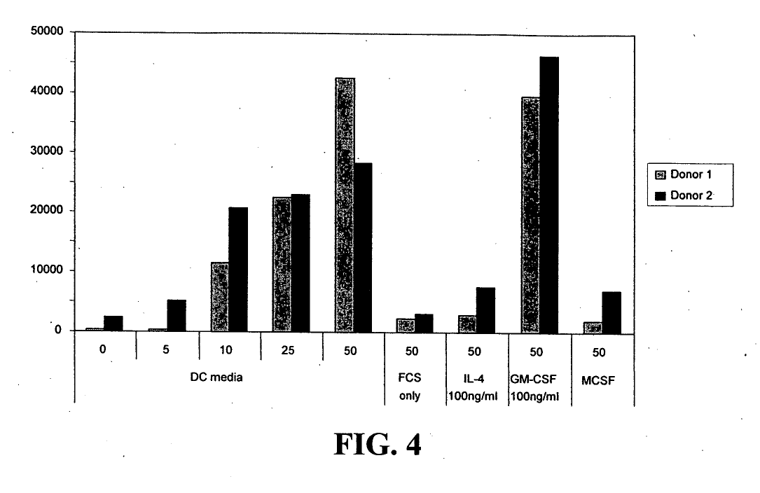

Peptide induced activation of ERK1/2 and p38 is dose dependent and demonstrates synergy with GM-CSF. GM-CSF, IL-4, or M-CSF (each at 100 ng/ml) was added concurrently with SEQ ID NO: 1 and phosphorylation of ERK1/2 was measured in freshly isolated human blood monocytes. ERK1/2 phosphorylation was evident when cells were treated with 50 μg/ml of SEQ ID NO: 1 (8.3 fold increase over untreated, n=9) but not at lower concentrations (n=2). In the presence of 100 ng/ml GM-CSF, SEQ ID NO: 1-induced ERK1/2 phosphorylation increased markedly (58 fold greater than untreated, n=5). Furthermore, in the presence of GM-CSF, activation of ERK1/2 occurred in response to concentrations of 5 and 10 μg/ml of SEQ ID NO: 1, respectively, in the two donors tested (FIG. 4). This demonstrates that SEQ ID NO: 1 induced activation of ERK1/2 occurred at a lower threshold in the presence of GM-CSF, a cytokine found locally at sites of infection.

FIG. 4 shows SEQ ID NO: 1 induced activation of ERK1/2 occurs at lower concentrations and is amplified in the presence of certain cytokines. When freshly isolated monocytes were stimulated in media containing both GM-CSF (100 ng/ml) and IL-4 (100 ng/ml) SEQ ID NO: 1 induced phosphorylation of ERK1/2 was apparent at concentrations as low as 5 μg/ml. This synergistic activation of ERK1/2 seems to be due primarily to GM-CSF.

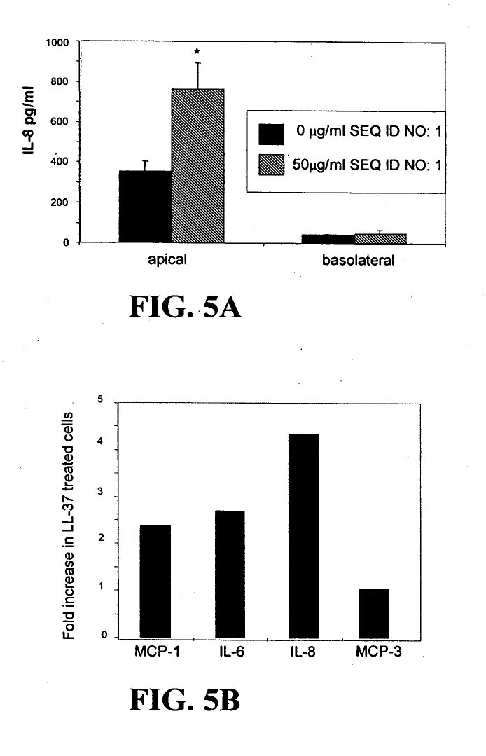

Activation of ERK1/2 leads to transcription of Elk-1 controlled genes and secretion of IL-8. IL-8 release is governed, at least in part, by activation of the ERK1/2 and p38 kinases. In order to determine if peptide could induce IL-8 secretion the human bronchial cell line, 16HBE4o-, was grown to confluency in Transwell filters, which allows for cellular polarization with the creation of distinct apical and basal surfaces. When the cells were stimulated with 50 μg/ml of SEQ ID NO: 1 on the apical surface for four hours a statistically significant increase in the amount of IL-8 released into the apical supernatant was detected (FIG. 5). To determine the downstream transcriptional effects of peptide-induced MAP kinase activation, the expression of genes known to be regulated by ERK1/2 or p38 was assessed by RT-PCR. RT-PCR was performed on RNA isolated from 16HBE4o-cells, treated for four hours with 50lg/ml of SEQ ID NO: 1 in the presence of serum, from two independent experiments. MCP-1 and IL-8 have been demonstrated to be under the transcriptional control of both ERK1/2 and p38, consistent with this they are up-regulated 2.4 and 4.3 fold respectively. Transcription of MCP-3 has not previously been demonstrated to be influenced by the activation of the mitogen activated protein kinases, consistent with this, expression is not affected by peptide treatment. (FIG. 5). These data are consistent with the hypothesis that activation of the activation of the ERK1/2 and p38 signaling pathways has functional effects on transcription of cytokine genes with immunomodulatory functions. The inset to FIG. 3B also demonstrates that peptide induced the phosphorylation of transcription facor Elk-1 in a serum dependent manner.

FIG. 5 shows peptide affects both transcription of various cytokine genes and release of IL-8 in the 16HBE4o-human bronchial epithelial cell line. Cells were grown to confluency on a semi-permeable membrane and stimulated on the apical surface with 50 μg/ml of SEQ ID NO: 1 for four hours. A) SEQ ID NO: 1 treated cells produced significantly more IL-8 than controls, as detected by ELISA in the supernatant collected from the apical surface, but not from the basolateral surface. Mean±SE of three independent experiments shown, asterisk indicates p=0.002.B) RNA was collected from the above experiments and RT-PCR was performed. A number of cytokine genes known to be regulated by either ERK1/2 or p38 were up-regulated upon stimulation with peptide. The average of two independent experiments is shown.

### EXAMPLE 13

### Modulation of an Inflammatory Response

The innate immune response is a dynamic system since it can be triggered by receptor. recognition of conserved bacterial components, initiating a broad inflammatory response to infectious agents, but must be able maintain homeostasis in the presence of commensal organisms, which contain many of these same conserved components. A delicate balance of pro- and anti-inflammatory mediators is vital for efficient functioning of the immune system under these disparate circumstances. In recent years, there has been speculation and some evidence implicating the sole human cathelicidin, SEQ ID NO: 1, in maintaining homeostasis, combating pathogenic challenge, and protecting against endotoxemia, an extreme inflammation-like condition (Devine D A, et al. Cationic peptides: distribution and mechanisms of resistance. Curr Pharm Des 2002; 8:703-14; Ciornei C D, et al. Antimicrobial and chemoattractant activity, Lipopolysaccharide neutralization, cytotoxicity, and inhibition by serum of analogs of human cathelicidin LL-37. Antimicrob Agents Chemother 2005; 49:2845-50). The data presented herein demonstrate that SEQ ID NO: 1 is an important component of human immunity that regulates the balance of pro- and anti-inflammatory molecules both under homeostatic conditions and during endotoxin challenge (i.e., infection situations).

Materials and Methods

Cell Isolation and Cell Lines—Human monocytic cells, THP-1 (Tsuchiya S, et al. Establishment and characterization of a human acute monocytic leukemia cell line (THP-1). Int J Cancer 1980; 26:171-6), were obtained from American type culture collection, ATCC® (TIB-202) and were grown in suspension in RPMI-1640 media (Gibco®, Invitrogen™ Life technologies, Burlington, ON), supplemented with 10% (v/v) heat inactivated fetal bovine serum (FBS), 2 mM L-glutamine and 1 mM sodium pyruvate (all from Invitrogen Life Technologies). Cultures were maintained at 37° C. in a humidified 5% (v/v) CO₂ incubator up to a maximum of six passages. THP-1 cells at a density of 1×10⁶ cells/ml were treated with 0.3 μg/ml phorbol 12-myristate 13-acetate (PMA; Sigma-Aldrich Canada, Oakville ON) for 24 hr (Tsuchiya S, et al. Induction of maturation in cultured human monocytic leukemia cells by a phorbol diester. Cancer Res 1982; 42:1530-6), inducing plastic-adherent cells that were further rested in complete RPMI-1640 medium for an additional 24 hr prior to stimulations with various treatments. Venous blood (20 ml) from healthy volunteers was collected in Vacutainer collection tubes containing sodium heparin as an anticoagulant (Becton Dickinson, Mississauga, ON) in accordance with UBC ethical approval and guidelines. Blood was diluted 1:1 with complete RPMI 1640 medium and separated by centrifugation over a Ficoll-Paqueg Plus (Amersham Biosciences, Piscataway, N.J., USA) density gradient. White blood cells were isolated from the buffy coat, washed twice in RPMI 1640 complete medium, and the number of peripheral blood mononuclear cells (PBMC) was determined by trypan blue exclusion. PBMC (5×10⁵) were seeded into 12-well tissue culture dishes (Falcon; Becton Dickinson) at 1×10⁶ cells/ml at 37° C. in 5% CO₂. All experiments using human THP-1 cells or PBMCs involved at least three biological replicates.

Stimulants, Reagents and Antibodies—LPS was isolated from P. aeruginosa H103 using the Darveau-Hancock method as previously described (Darveau RP, et al. Procedure for isolation of bacterial lipopolysaccharides from both smooth and rough Pseudomonas aeruginosa and Salmonella typhimurium strains. J Bacteriol 1983; 155:831-8). Briefly, P. aeruginosa was grown overnight in LB broth at 37° C. Cells were collected and washed and the isolated LPS pellets were extracted with a 2:1 chloroform:methanol solution to remove contaminating lipids. Purified LPS samples were quantitated using an assay for the specific sugar 2-keto-3-deoxyoctosonic acid (KDO assay) and then resuspended in endotoxin-free water (Sigma-Aldrich).

TLR2 agonists lipoteichoic acid (LTA) from S. aureus and a synthetic tripalmitoylated lipopeptide, Pam₃CSK4, were purchased from InvivoGen (San Diego, Calif., USA). TLR9 agonist CpG oligodeoxynucleotide #2007 (Krieg AM. CpG motifs in bacterial DNA trigger direct B-cell activation. Nature 1995; 374:546-9) was a gift from Dr. Lorne Babuik (Vaccine and Infectious Disease org., SK, Canada). Recombinant human TNFα and recombinant human ILL1β were obtained from Research Diagnostics Inc., (Flanders, N.J., USA). All reagents were tested for endotoxin and reconstituted in endotoxin-free water. LTA from S. aureus used in this study had 1.25 EU of endotoxin/μg of LTA. Polymyxin B was purchased from InvivoGen, Actinomycin D (transcriptional inhibitor) was purchased from Calbiochem-Novabiochem Corporation (La Jolla, Calif.) and Monensin (inhibitor of protein secretion) was purchased from eBiosciences., CA, USA. A cationic peptide, SEQ ID NO: 1, was synthesized using F-moc chemistry at the Nucleic Acid/Protein Synthesis Unit, University of British Columbia (Vancouver, BC, Canada). The synthetic peptide was re-suspended in endotoxin-free water and stored at −20° C. until flurther use.

Rabbit polyclonal antibodies against the NFκB subunits p105/p50, p65 and RelB were purchased from Cell Signaling Technologies (Mississauga, ON, Canada). Rabbit polyclonal antibody against the NFκB subunit c-Rel was purchased from Chemicon International (Temecula, CA, USA) and mouse IgG2a monoclonal antibody against NFκB subunit p100/p52 was purchased from Upstate Cell Signaling Solutions (Lake Placid, N.Y., USA). HRP-conjugated goat anti-rabbit and anti-mouse IgG antibodies were purchased from Cell Signaling Technologies and Amersham Biosciences respectively.

Treatment with inflammatory stimuli, peptide or inhibitors—THP-1 cells or PBMC were stimulated with LPS (10 or 100 ng/ml), LTA (1 μg/ml), Pam₃CSK4 (100 ng/ml), CpG-ODN 2007 (2 μg/ml), recombinant human TNFα (50 ng/ml) or recombinant human IL1β (50 ng/ml) for 1, 2, 4, or 24 hours. SEQ ID NO: 1 (0.5-50 lg/ml) was added simultaneously or 30 min after addition of the stimulants. Alternatively, cells were stimulated with SEQ ID NO: 1 (20 μg/ml) for 30 min, washed with RPMI complete media to remove the peptide and then stimulated with LPS (100 ng/ml). Polymyxin B (0.1 mg/ml), actinomycin D (4 μg/ml), or monensin (working concentration as per the manufacturer's instructions) were added to the THP-1 cells 30 min prior to stimulants.

Detection ofcytokines—Following incubation of the cells under various treatment regimens, the tissue culture supernatants were centrifuged at 1000× g for 5 min, then at 10,000×g for 2 min to obtain cell-free samples. Supernatants were aliquoted and then stored at −20° C. prior to assay for various cytokines. TNFα and IL-8 secretion were detected with a capture ELISA (eBioscience and BioSource International Inc., CA, USA respectively) using either tissue culture supernatants or the nuclear and cytoplasmic extracts (see below) as per the experimental design. All assays were performed in triplicate. The concentration of the cytokines in the culture medium was quantified by establishing a standard curve with serial dilutions of the recombinant human TNFα or IL-8 respectively. Alternatively, five cytokines (GM-CSF, IL-1β, IL-6, IL-8 and TNFα) were measured simultaneously using the Human Cytokine 5-Plex kit from Biosource International Inc., (Medicorp Inc., Montreal, Canada) as per the manufacturer's instructions. The multiplex bead immunoassays were analyzed using Luminex 100™ StarStation software (Applied Cytometry Systems, Sacramento, CA, USA).

RNA extraction, amplification and hybridization to DNA microarrays—RNA was isolated from THP-1 cells with RNeasy Mini kit, treated with RNase-Free DNase (Qiagen Inc., Canada) and eluted in RNase-free water (Ambion Inc., Austin, Tex., USA) as per the manufacturer's instructions. RNA concentration, integrity and purity were assessed by Agilent 2100 Bioanalyzer using RNA 6000 Nano kits (Agilent Technologies; USA). RNA was (reverse) transcribed with incorporation of amino-allyl-UTP (aa-UTP) using the MessageAmpII™ amplification kit, according to the manufacturer's instructions, then column purified and eluted in nuclease-free water. Column purified samples were labeled with mono-functional dyes, Cyanine-3 and Cyanine-5 (Amersham Biosciences), according to manufacturer's instructions, and then purified using the Mega Clear kit (Ambion). Yield and fluorophore incorporation was measured using Lambda 35 UV/VIS fluorimeter (PerkinElmer Life and Analytical Sciences, Inc., USA). Microarray slides were printed with the human genome 21K Array-Ready Oligo Set™ (Qiagen Inc., USA) at The Jack Bell Research Center (Vancouver, BC, Canada). The slides were pre-hybridized for 45 min at 48° C. in pre-hybridization buffer containing 5×SSC (Ambion), 0.1% (w/v) SDS and 0.2% (w/v) BSA. Equivalent (20 pmol) cyanine labeled samples from control and treated cells were then mixed and hybridized on the array slides, in Ambion SlideHyb™ buffer#2 (Ambion) for 18 hr at 37° C. in a hybridization oven. Following hybridization, the slides were washed twice in 1×SSC/0.1% sodium dodecyl sulphate (SDS) for 5 min at 65° C., then twice in 1×SSC and 0.1×SSC for 3 min each at 42° C. Slides were centrifugated for 5 min at 1000×g, dried and scanned using ScanArray™ Express software/scanner (scanner and software by Packard BioScience BioChip Technologies) and the images were quantified using ImaGene™ (BioDiscovery Inc., El Segundo, Calif., USA).

Analysis of DNA Microarrays—Assessment of slide quality, normalization, detection of differential gene expression and statistical analysis was carried out with ArrayPipe (version 1.6), a web-based, semi-automated software specifically designed for processing of microarray data (Hokamp K, et al. ArrayPipe: a flexible processing pipeline for microarray data. Nucleic Acids Res 2004; 32(Web Server issue):W457-9) (www.pathogenomics.ca/arraypipe). The following processing steps were applied: 1) flagging of markers, 2) subgrid-wise background correction, using the median of the lower 10% foreground intensity as an estimate for the background noise, 3) data-shifting, to rescue negative spots, 4) printTip LOESS normalization, 5) merging of technical replicates, 6) two-sided one-sample Student t-test on the log₂-ratios within each treatment group, 7) averaging of biological replicates to yield overall fold-changes for each treatment group. Further, the gene expression data was overlaid on molecular interaction networks using Cytoscape (Shannon P, et al. Cytoscape: a software environment for integrated models of biomolecular interaction networks. Genome Res 2003; 13:2498-504). Interactions networks were custom built from manually curated data and information contained within the Transpath pathway database (Krull M, et al. TRANSPATH: an integrated database on signal transduction and a tool for array analysis. Nucleic Acids Res 2003; 31:97-100). The false discovery rate of selecting differentially expressed genes from microarray analysis was estimated at 35%, based on Beta Uniform Mixture model (Pounds S, et al. Estimating the occurrence of false positives and false negatives in microarray studies by approximating and partitioning the empirical distribution of p-values. Bioinformatics 2003; 19:1236-42) and Q-Value model (Storey JD. A direct approach to false discovery rates. Journal of the Royal Statistical Society 2002; 64:479-498). This was consistent with the confirmation, using qPCR, at 4 different time points, of array results for 14 of 20 genes (70%) selected for follow-up.

Quantitative real-time PCR (qPCR)—Differential gene expression identified by microarray analysis was validated using quantitative real-time PCR (qPCR) using SuperScriptTM III Platinum® Two-Step qRT-PCR Kit with SYBR® Green (Invitrogen Life Technologies), as per the manufacturer's instructions, in the ABI PRISMS® 7000 sequence detection system (Applied Biosystems, Foster city, Calif., USA). Briefly, 1 μg of total RNA was reverse transcribed in a 20 μl reaction volume for 50 min at 42° C., the reaction was terminated by incubating for 5 min at 85° C. and then digested for 30 min at 37° C. with RNAse H. The PCR reaction was carried out in a 12.5 μl reaction volume containing 2.5 μl of 1/10 diluted cDNA template. A melting curve was performed to ensure that any product detected was specific to the desired amplicon. Fold changes were calculated after normalization to endogenous GAPDH and using the comparative Ct method (Pfaffl MW. A new mathematical model for relative quantification in real-time RT-PCR. Nucleic Acids Res 2001; 29:No. 9 e45). The primers used for qRT-PCR are reported in Table 65.

TABLE 65
Sequence of primers (human) used for qPCR

| | | |
|---|---|---|
| Gene | Forward primer (5′-3′) | Reverse Primer (5′-3′) |

| | | |
|---|---|---|
| CCL4 | CTTTTCTTACACCGCGAGGAA | GCAGAGGCTGCTGGTCTCAT |
| | (SEQ ID NO: 67) | (SEQ ID NO: 68) |
| CCL20 | TGACTGCTGTCTTGGATACACAGA | TGATAGCATTGATGTCACAGCCT |
| | (SEQ ID NO: 69) | (SEQ ID NO: 70) |
| CXCL1 | GCCAGTGCTTGCAGACCCT | GGCTATGACTTCGGTTTGGG |
| | (SEQ ID NO: 71) | (SEQ ID NO: 72) |
| IL-8 | GACCACACTGCGCCAACAC | CTTCTCCACAACCCTCTGCAC |
| | (SEQ ID NO: 73) | (SEQ ID NO: 74) |
| GAPDH | GTCGCTGTTGAAGTCAGAGG | GAAACTGTGGCGTGATGG |
| | (SEQ ID NO: 75) | (SEQ ID NO: 76) |
| IL-10 | GGTTGCCAAGCCTTGTCTGA | AGGGAGTTCACATGCGCCT |
| | (SEQ ID NO: 77) | (SEQ ID NO: 78) |
| TNF-α | TGGAGAAGGGTGACCGACTC | TCCTCACAGGGCAATGATCC |
| | (SEQ ID NO: 79) | (SEQ ID NO: 80) |
| TNFAIP2 | CTACCAGCGCGCCTTTAATG | TCCGGAAGGACAGGCAGTT |
| | (SEQ ID NO: 81) | (SEQ ID NO: 82) |
| TNFAIP3 | CTGCCCAGGAATGCTACAGATAC | CAGGGTCACCAAGGGTACAAA |
| | (SEQ ID NO: 83) | (SEQ ID NO: 84) |
| TNIP3 | TGAAAGAAAGGTAGCAGAGCTGAA | CCGCGTGCTGAGGAATCT |
| | (SEQ ID NO: 85) | (SEQ ID NO: 86) |
| BIRC3 | AAAGCGCCAACACGTTTGA | AGGAACCCCAGCAGGAAAAG |
| | (SEQ ID NO: 87) | (SEQ ID NO: 88) |
| NF-κB1 | CTTAGGAGGGAGAGCCCACC | TTGTTCAGGCCTTCCCAAAT |
| | (SEQ ID NO: 89) | (SEQ ID NO: 90) |
| RELA | TAGGAAAGGACTGCCGGGAT | CCGCTTCTTCACACACTGGA |
| | (SEQ ID NO: 91) | (SEQ ID NO: 92) |
| RELB | TGGGCATTGACGCCTACAAC | TGGGTCCCTGAAGAACCATCAGGAAGTAGA |
| | (SEQ ID NO: 93) | (SEQ ID NO: 94) |
| NF-κBIA | GGTGAAGGGAGACCTGGCTT | GTGCCTCAGCAATTTCTGGC |
| | (SEQ ID NO: 95) | (SEQ ID NO: 96) |

Nuclear and Cytoplasmic Extracts—THP-1 cells (3×10⁶) seeded into 60 mm² petri dishes (VWR International, Mississauga, ON) were pre-treated with inhibitors for 30 min, and then stimulated with agonists or peptide for 30 min or 60 min. Cells were subsequently treated with Versene for 10 min at 37° C. in 5% CO₂ (to detach adherent cells) then washed twice with ice-cold phosphate buffered saline. Cytoplasmic and nuclear extracts were isolated using NE-PER® Nuclear and Cytoplasmic Extraction Reagents Kit (Pierce Biotechnology, Rockford, Ill., USA) according to the manufacturer's instructions. The protein concentration of the extracts was quantified using a Bicinchoninic Acid (BCA) Protein Assay (Pierce Biotechnology) and the extracts were stored at −80° C. until fturther use.

Translocation of NFκB subunits—Equivalent nuclear extracts (5-10 μg) were resolved on a 7.5% SDS-polyacrylamide gel (SDS-PAGE) and transferred to polyvinylidene difluoride (PVDF) Immobilon-P membranes (Millipore Canada Ltd., Mississauga, ON). Equivalent protein loading was verified by staining PVDF membranes with Blot-Fast-Stain™ (Chemicon International) according to the manufacturer's instructions. Subsequently, the PVDF membranes were incubated with anti-p105/p50, anti-p65, anti-c-Rel, anti-Rel B or anti-p100/p52 antibodies at 1/1000 dilution in TBST (20 mM Tris pH 7.5, 150 mM NaCl, 0.1% Tween 20) containing 5% skimmed milk powder (TBST/milk) for 1 hr. Membranes were washed for 1 hour in TBST and then incubated with a 1/5000 dilution of HRP-conjugated goat anti-mouse or anti-rabbit Ab (in TBST/milk) for 30 min. The membranes were incubated for 30 to 60 min in TBST and developed with chemiluminescence peroxidase substrate (Sigma-Aldrich), according to manufacturer's instructions. Alternatively, equivalent nuclear extracts (2.5-10 ug) were analyzed for NFκB subunits p50 or p65 by StressXpress NFκB p50 or p65 ELISA kits (Stressgen Bioreagents, Victoria, BC, Canada) according to manufacturer's instructions. Luminescence was detected with SpectraFluor Plus Multifunction Microplate Reader (Tecan Systems Inc., SJ, USA).

Results

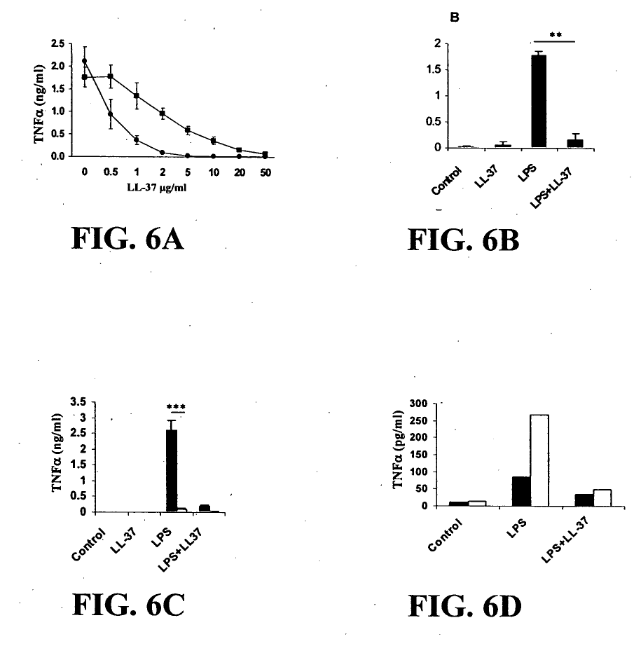

Low, physiological concentrations of SEQ ID NO: 1 suppress LPS-indauced secretion of the pro-inflarmnnatoiry cytokine TNFα. SEQ ID NO: 1 is found at mucosal surfaces at concentrations of around 2.5 to 5 μg/ml in adults and up to 20 μg/ml in infants (Schaller-Bals S, et al. Increased levels of antimicrobial peptides in tracheal aspirates of newborn infants during infection. Am J Respir Crit Care Med 2002; 165:992-5). Previous studies indicated that it has the ability to down-regulate pro-inflammatory cytokines in isolated monocytic cells (Bowdish D M, et al. Immunomodulatory activity of small host defense peptides. Antimicrob Agents Chemother 2005; 49:1727-32). To determine the lowest dose of SEQ ID NO: 1 that exhibited anti-endotoxin activity, THP-1 cells were stimulated with LPS (10 and 100 ng/ml) in the absence or presence of SEQ ID NO: 1 added simultaneously at concentrations ranging from 0.5 to 50 μg/ml for a period of 4 hours in complete RPMI cell culture media (i.e., which contains physiological salt concentrations). Tissue culture supernatants were assayed by ELISA for the presence of the pro-inflammatory cytokine TNFα (FIG. 6A). Very low concentrations (<1 μg/ml) of SEQ ID NO: 1 inhibited TNFα release from LPS-induced cells, demonstrating that physiological concentrations of SEQ ID NO: 1 exhibit anti-endotoxin activity. The anti-endotoxin effect of SEQ ID NO: 1 was more pronounced when the cells were stimulated with 10 ng/ml of LPS, a concentration at the lower level of concentrations used by investigators to mimic TLR signaling responses, but considerably higher than circulating endotoxin concentrations in septic patients (Opal S M, et al. Relationship between Plasma Levels of Lipopolysaccharide (LPS) and LPS-Binding Protein in Patients with Severe Sepsis and Septic Shock http://wwwjournals.uchicago.edu/JID/journal/issues/v180n5/990373/990373.text.html-fn1#fn1 J Infect Dis 1999; 180:1584-9). Under these conditions, 0.5 fg/ml of SEQ ID NO: 1 inhibited 50% of LPS-induced TNFα release. This inhibitory effect increased to ≧80% with a dose of 1 μg/ml of SEQ ID NO: 1, and TNFα was reduced to background levels with 2 μg/ml of SEQ ID NO: 1. In the presence of LPS at a higher concentration (100 ng/ml), 2 μg/ml of SEQ ID NO: 1 was required to inhibit 50% of TNFα released into the tissue culture supernatant. Higher concentrations (20 μg/ml) of SEQ ID NO: 1 caused ≧95% inhibition of TNFα release. These results indicated that physiological concentrations of SEQ ID NO: 1 exhibit an anti-endotoxin effect on LPS present at low and high concentrations. The anti-endotoxin effect of SEQ ID NO: 1 was similarly observed in PBMCs (FIG. 6B), for which SEQ ID NO: 1 (20 μg/ml) inhibited >91% of LPS (100 ng/ml) induced TNF-α. Subsequent mechanistic studies employed 100 ng/ml of LPS, at which concentrations more robust transcriptional up-regulation responses were observed, and 20 μg/ml of SEQ ID NO: 1, which was not cytotoxic to primary cells (Bowdish D, et al. The human cationic peptide LL-37 induces activation of the extracellular signal-regulated kinase and p38 kinase pathways in primary human monocytes. J Immunol 2004; 172:3758-65) or THP-1 cells as determined by LDH (lactose dehydrogenase) release and MTT (3-(4,5-dimethylthiazol-2-yl)-2,5-diphenyltetrazolium bromide) assay (data not shown).

To gain further insight into the mode of inhibition exerted by SEQ ID NO: 1, TNFα production and release was monitored in the supernatants of LPS-stimulated THP-1 cells treated with the transcriptional inhibitor actinomycin D. Four μg/ml of actinomycin D was used since this concentration was required for inhibition, by more than 96% within 1 hour of treatment, of LPS-induced transcription of the genes for both the cytokine TNFα and the pro-inflammatory TNFα-inducible protein 2 (TNFAIP2) (monitored by qPCR, data not shown). Actinomycin D reduced the level of TNFα release by 97.6% (FIG. 6C), indicating that LPS largely induced de novo expression of TNFα as opposed to processing and release of intracellular pools of pro-form TNFα. Moreover, the use of monensin as an inhibitor of TNFα secretion led to accumulation of TNFα within cells after LPS stimulation for 60 min (FIG. 6D). However SEQ ID NO: 1 by itself did not similarly lead to the accumulation of TNFα inside cells, indicating that it also prevented TNFα expression at the protein level rather than blocking secretion.

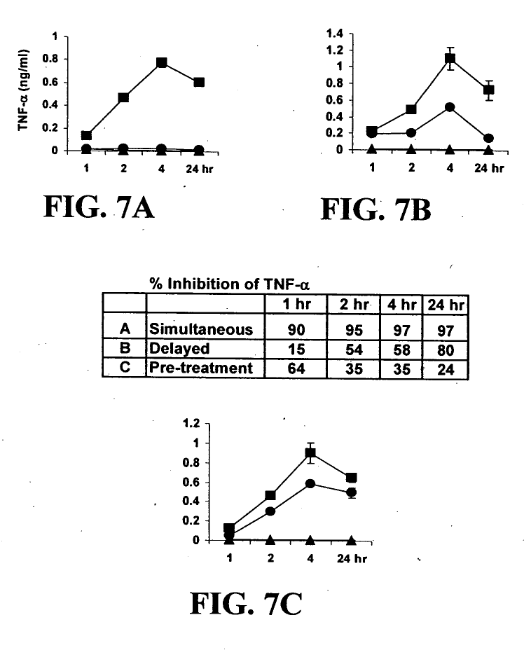

The sustained presence of SEQ ID NO: 1 inhibits TNFα release. To determine the kinetics of the anti-endotoxin effect, the supernatant from THP-1 cells was monitored for TNFα after 1, 2, 4 and 24 hr of stimulation with LPS (100 ng/ml) in absence or presence of SEQ ID NO: 1 (20 μg/ml). When the peptide and LPS were added simultaneously, the release of TNFα was substantially inhibited (90 to 97%) by SEQ ID NO: 1 at all time points (FIG. 7A). When SEQ ID NO: 1 was added 30 min after LPS addition, TNFα secretion was reduced more than 50% at 2 and 4 hr post LPS treatment and by 80% after 24 hr (FIG. 7B) consistent with previous observations in mouse macrophages (Scott MG, et al. The human antimicrobial peptide SEQ ID NO: 1 is a multifunctional modulator of innate immune responses. J Imnmunol 2002; 169:3883-91). In contrast, when the cells were pre-treated with SEQ ID NO: 1 for 30 min, washed and stimulated with LPS, TNFα secretion was substantially (64%) reduced after 1 hr, but this declined to only 24 to 35% at subsequent time points (FIG. 7C). This indicated that a sustained presence of SEQ ID NO: 1 was required to exhibit a maximal anti-endotoxin effect.

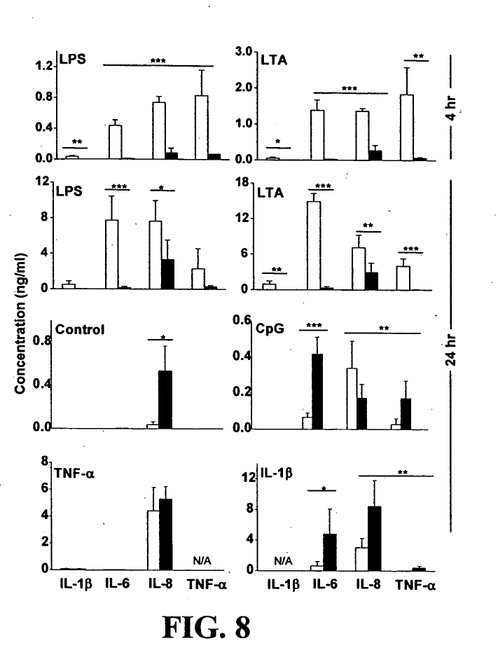

SEQ ID NO: 1 suppresses TLR-induced cytokine secretion by PBMC. PBMC were treated with agonists of TLR2 (LTA, PAM₃CSK4), TLR4 (LPS), TLR9 (CpG), and the inflammatory cytokines TNFα and IL-1β, to determine if SEQ ID NO: 1 could suppress cytokine secretion induced by inflammatory stimuli LPS and other agonists in primary cells. Cytokine production was analyzed by Luminex 100™ StarSystem using the human 5-Plex cytokine kit to monitor IL-1β, IL-6, IL-8 and TNFα in the culture supernatants. The cytokine profile of stimulated PBMC in the presence or absence of SEQ ID NO: 1 was monitored after 4 or 24 hours of treatment. The release of all 4 cytokines was significantly reduced by SEQ ID NO: 1 in both LPS- and LTA-stimulated cells after 4 hr of treatment, and this anti-inflammatory activity was sustained over 24 hr (FIG. 8). Effects on IL-8 production were more modest, as anticipated, since SEQ ID NO: 1 has the ability to induce IL-8 production (Scott MG, et al. The human antimicrobial peptide LL-37 is a multifunctional modulator of innate immune responses. J Immunol 2002; 169:3883-91). In addition, SEQ ID NO: 1 reduced IL-1β, IL-6, IL-8 and TNFα production by TLR2-agonist PAM₃CSK4-stimulated PBMC after 4 or 24 hr of treatment, by approximately 30-50%, (Table 66). These data show that SEQ ID NO: 1 significantly reduced the production of pro-inflammatory cytokines resulting from activation of TLR2 or TLR4 (Table 66). SEQ ID NO: 1 also reduced, by ˜50%, IL-8 secretion by PBMC stimulated with the TLR9 agonist CpG for 24 hr (FIG. 8; Table 66).

In contrast, SEQ ID NO: 1 enhanced TNFα and IL-6 production by CpG-stimulated PBMC and IL-6, IL-8 and (modestly) TNFα by PBMC stimulated with IL-1β (FIG. 8; Table 66). Conversely, SEQ ID NO: 1 had no effect on TNFα induced cytokine production. These results indicate that the SEQ ID NO: 1 was anti-inflammatory in response to selected TLR ligands, and that it was likely modulating innate immune pathways rather than simply suppressing some step in the main TLR to NFκB pathway.

Table 66 lists percent inhibition or enhancement of agonist-induced cytokine production by SEQ ID NO: 1. PBMC were incubated alone or with TLR agonists (LPS, LTA, CpG) or inflammatory cytokines (TNFα, IL-1β) for 4 or 24 hr in the presence or absence of SEQ ID NO: 1. The concentration of IL-1β, IL-6, IL-8 and TNFα released in the tissue culture supernatant is reported. The percent inhibition of IL-1β, IL-6, IL-8 and TNFα in the presence of SEQ ID NO: 1±the standard deviation of 3 biological repeats is reported, as well as the fold enhancement of cytokine production in the presence of SEQ ID NO: 1±the standard deviation of 3 biological repeats.

| | Agonist | | | | | | | | | | | |
|---|---|---|---|---|---|---|---|---|---|---|---|---|
| | Cells Only | | | TNF-α | | | IL-1β | | | LPS | | |
| | Ave pg/ml | | | Ave pg/ml | | | Ave pg/ml | | | Ave pg/ml | | |
| | −SEQ | +SEQ | Fold Inc.or | −SEQ | +SEQ | Fold Inc. | −SEQ | +SEQ | Fold Inc. | −SEQ | +SEQ | Fold Inc. |
| | ID | ID | % Inh. | ID | ID | or % Inh. | ID | ID | or % Inh. | ID | ID | or % Inh. |
| | NO: 1 | NO: 1 | (Ave ± SD) | NO: 1 | NO: 1 | (Ave ± SD) | NO: 1 | NO: 1 | (Ave ± SD) | NO: 1 | NO: 1 | (Ave ± SD) |
| Release by | | | | | | | | | | | | |
| 4 hr | | | | | | | | | | | | |
| IL-1β | <9 | | — | 74 | 71 | 1.0 ± 0.1 | N/A | | | 34 | <9 | >81.9 ± 17.7 |
| IL-6 | <7 | | — | <7 | | — | 39 | 53 | 1.2 ± 0.4 | 435 | <7 | >98.4 ± 0.8 |
| IL-8 | 15 | 32 | 2.1 ± 0.1 | 54 | 90 | 1.9 ± 0.7 | 124 | 333 | 2.4 ± 1.2 | 738 | 84 | 89.1 ± 7.7 |
| TNF-α | <16 | | — | N/A | | | 97 | 9 | N/A | 830 | 73 | 96.2 ± 2.2 |
| Release by | | | | | | | | | | | | |
| 24 hr | | | | | | | | | | | | |
| IL-1β | <9 | | — | 94 | 95 | 1.0 ± 0.2 | N/A | | | 512 | 14 | 99.1 ± 1.3 |
| IL-6 | <7 | | — | 9 | 13 | 1.7 ± 0.6 | 645 | 4815 | 8.1 ± 1.4 | 7734 | 170 | 97.9 ± 1.3 |
| IL-8 | 37 | 532 | 10.3 ± 4.0 | 4410 | 5320 | 1.4 ± 0.6 | 3034 | 8452 | 2.9 ± 0.9 | 7620 | 3332 | 74.7 ± 15.8 |

| | | | | | | | | | |
|---|---|---|---|---|---|---|---|---|---|
| TNF-α | <16 | — | N/A | 20 | 451 | 23.6 ± 12.6 | 2334 | 303 | 78.9 ± 18.0 |

| | Agonist | | | | | | | | |
|---|---|---|---|---|---|---|---|---|---|
| | | | | | | | CpG | | |
| | LTA | | | PAM3 | | | Fold Inc. | | |
| | Ave pg/ml | | | Ave | | | or % Inh. | | |
| | −SEQ | | Fold Inc. or | pg/ml | | | (Ave ± SD) | | |
| | ID | +SEQ ID | % Inh. | −SEQ ID | +SEQ ID | Fold Inc. or % | −SEQ | +SEQ ID | Fold Inc. or % |
| | NO: 1 | NO: 1 | (Ave ± SD) | NO: 1 | NO:1 | Inh. (Ave ± SD) | ID NO: 1 | NO: 1 | Inh. (Ave ± SD) |
| Release by | | | | | | | | | |
| 4 hr | | | | | | | | | |
| IL-1β | 53 | <9 | >87.0 ± 14.5 | 34 | <9 | >81.9 ± 17.7 | <9 | | — |
| IL-6 | 1391 | 24 | 98.3 ± 0.8 | 435 | <7 | >98.4 ± 0.8 | <7 | 17.0 | — |
| IL-8 | 1366 | 273 | 79.8 ± 11.8 | 738 | 84 | 89.1 ± 7.7 | 20 | 34 | 1.7 ± 0.6 |
| TNF-α | 1836 | 66 | 96.3 ± 0.6 | 830 | 73 | 96.2 ± 2.2 | 28 | 34 | 3.5 ± 2.6 |
| Release by | | | | | | | | | |
| 24 hr | | | | | | | | | |
| IL-1β | 969 | <9 | >99.4 ± 0.6 | 512 | 14 | 99.1 ± 1.3 | <9 | | 6.6 ± 1.7 |
| IL-6 | 14887 | 318 | 97.9 ± 1.5 | 7734 | 170 | 97.9 ± 1.3 | 66 | 417 | — |
| IL-8 | 7108 | 2928 | 58.8 ± 23.1 | 7620 | 3332 | 74.7 ± 15.8 | 339 | 174 | 48.6 ± 1.0 |
| TNF-α | 4040 | 39 | 99.2 ± 0.6 | 2334 | 303 | 78.9 ± 18.0 | 28 | 171 | 17.6 ± 20.5 |

LPS-induced gene expression profile is altered by SEQ ID NO: 1. Human 21K oligo-based DNA microarrays were probed to elucidate the impact of SEQ ID NO: 1 on LPS stimulation of gene responses in human monocytic cells. Transcriptional responses were analyzed following 1, 2, 4 and 24 hr of stimulation to provide a temporal profile of gene expression in monocytes equivalent to the early, intermediate and late stages of innate immune responses. Microarray analyses were performed in duplicate from three independent biological replicates. Statistically significant, differentially expressed genes were defined as those with a fold change of at least 1.5 with a Student's t-test p-value≦0.05 (MIAME compliant data was deposited to ArrayExpress). The number of differentially expressed genes was greatest at the 2 and 4 hr time points. Over the monitored time period, 561 and 410 genes were differentially regulated in the presence of LPS, without or with SEQ ID NO: 1 respectively. Of the 561 genes that were differentially expressed in LPS-stimulated cells, only 39 (˜7%) were identified as being up-regulated in cells stimulated with LPS in the presence of SEQ ID NO: 1 (Table 67). At least 163 genes that were upregulated in cells stimulated with LPS (i.e., proinflaninatory genes) were suppressed in the presence of SEQ ID NO: 1 (Table 68). This indicates that SEQ ID NO: 1 effectively suppressed the induction of a large subset of LPS-responsive genes, but maintained a modest subset of genes that function in promoting some aspects of inflammation or anti-inflammatory response.

TABLE 67
List of 39 genes differentially expressed upon stimulation by LPS and
remaining up-regulated in the presence of SEQ ID NO: 1, as detected by microarray
analysis at one or more time points.

| | LPS_1 hr | | LPS_2 hr | | LPS_4 hr | | LPS_24 hr | |
|---|---|---|---|---|---|---|---|---|
| | Fold | | Fold | | Fold | | Fold | |
| Gene Name | change | p-value | change | p-value | change | p-value | change | p-value |
| ZNF83 | 4.21 | 0.01 | 1.59 | 0.59 | −1.42 | 0.73 | −1.20 | 0.92 |
| NFKBIA | 1.71 | 0.01 | 2.22 | 0.35 | 1.88 | 0.11 | 1.53 | 0.05 |
| Q9P188 | 1.69 | 0.02 | 1.13 | 0.24 | 1.66 | 0.09 | 3.30 | 0.24 |
| INVS | 1.69 | 0.02 | −1.36 | 0.60 | 1.51 | 0.73 | 1.44 | 0.87 |
| DIAPH1 | 1.77 | 0.02 | −1.49 | 0.87 | 1.81 | 0.13 | −1.15 | 0.58 |
| IER3 | 1.58 | 0.03 | 2.26 | 0.10 | 1.99 | 0.03 | 2.92 | 0.12 |
| Q9H640 | 1.62 | 0.04 | 1.43 | 0.44 | −1.45 | 0.53 | −1.93 | 0.36 |
| GBP2 | 1.32 | 0.05 | 2.10 | 0.01 | 2.38 | 0.02 | 1.04 | 0.34 |
| NANS | 1.13 | 0.05 | 1.65 | 0.04 | 1.62 | 0.07 | 1.81 | 0.00 |
| Q86XN7; | 2.67 | 0.06 | 8.01 | 0.04 | 7.45 | 0.03 | −1.02 | 0.20 |
| Q9H9M1 | | | | | | | | |
| TNFAIP3 | 2.47 | 0.07 | 3.35 | 0.05 | 3.71 | 0.04 | 1.33 | 0.23 |
| Q96MJ8; | 1.74 | 0.08 | 4.01 | 0.01 | 1.90 | 0.58 | 1.65 | 0.05 |
| Q9BSE2 | | | | | | | | |
| Q9H753 | 2.29 | 0.08 | 3.91 | 0.02 | 2.55 | 0.77 | 1.02 | 0.75 |
| NTNG1 | 3.75 | 0.08 | −1.46 | 0.27 | 1.05 | 0.41 | 1.52 | 0.02 |
| INHBE | 1.58 | 0.09 | 1.84 | 0.05 | −1.07 | 0.64 | 1.07 | 0.73 |
| BCL6 | 1.76 | 0.12 | 1.67 | 0.03 | 1.73 | 0.04 | 1.05 | 0.25 |
| CXCL1 | 2.54 | 0.12 | 4.26 | 0.05 | 1.98 | 0.11 | 1.30 | 0.39 |
| EHD1 | 1.80 | 0.13 | 3.42 | 0.05 | 3.17 | 0.02 | 1.88 | 0.08 |
| RELB | 1.16 | 0.14 | 2.16 | 0.05 | 2.80 | 0.02 | 1.42 | 0.22 |
| HRK | 1.82 | 0.15 | 1.58 | 0.23 | 3.15 | 0.50 | 2.72 | 0.05 |
| CCL4 | 2.03 | 0.15 | 2.43 | 0.01 | 1.71 | 0.09 | 1.20 | 0.15 |
| SESN2 | 1.26 | 0.17 | 2.47 | 0.05 | 2.66 | 0.03 | −1.33 | 0.57 |
| NAB1 | 1.22 | 0.17 | 1.67 | 0.05 | 2.46 | 0.06 | 1.17 | 0.31 |
| EBI3 | 1.18 | 0.19 | 5.59 | 0.06 | 1.78 | 0.12 | −1.06 | 0.40 |
| DDX21 | 1.26 | 0.23 | 1.51 | 0.06 | 2.74 | 0.15 | −1.08 | 0.35 |
| XBP1 | 1.76 | 0.23 | 1.80 | 0.05 | 1.32 | 0.05 | 1.39 | 0.08 |
| SULRP1; ARS | 1.56 | 0.25 | 2.10 | 0.17 | 1.33 | 0.23 | 1.80 | 0.05 |
| HDAC10 | 2.19 | 0.31 | 1.35 | 0.19 | 1.60 | 0.06 | 1.13 | 0.25 |
| MEP1A | −1.23 | 0.39 | 1.08 | 0.72 | −1.16 | 0.59 | 2.47 | 0.02 |
| RAP2C | 1.34 | 0.43 | 1.70 | 0.03 | 2.61 | 0.04 | 1.37 | 0.09 |
| GYS1 | −1.30 | 0.47 | −1.01 | 0.54 | 2.17 | 0.03 | 2.26 | 0.51 |
| RARRES3 | 1.29 | 0.48 | −2.19 | 0.57 | 1.01 | 0.66 | 1.77 | 0.05 |
| PPY | 1.19 | 0.49 | 1.71 | 0.61 | 1.58 | 1.00 | 4.28 | 0.02 |
| NFKB1 | 1.16 | 0.75 | 1.72 | 0.01 | 1.89 | 0.03 | −1.12 | 0.97 |
| MTL4_HUMAN | 1.10 | 0.81 | 1.52 | 0.04 | 2.22 | 0.23 | −1.07 | 0.88 |
| Q9H040 | −1.62 | 0.82 | −1.02 | 0.72 | 1.58 | 0.01 | 1.71 | 0.43 |
| Q9NUP6 | 1.51 | 0.99 | 1.31 | 0.28 | 1.25 | 0.12 | 6.86 | 0.06 |
| | LPS + SEQ | | LPS + SEQ | | LPS + SEQ | | LPS + SEQ | |
| | ID NO: 1 | | ID NO: 1 | | ID NO: 1 | | ID NO: 1 | |
| | 1 hr | | 2 hr | | 4 hr | | 24 hr | |
| | Fold | | Fold | | Fold | | Fold | |
| Gene Name | change | p-value | change | p-value | change | p-value | change | p-value |
| ZNF83 | 2.02 | 0.03 | 1.08 | 0.65 | 1.17 | 0.41 | −1.37 | 0.38 |
| NFKBIA | 1.94 | 0.03 | 2.36 | 0.01 | 1.50 | 0.23 | 1.30 | 0.02 |
| Q9P188 | 1.58 | 0.04 | 1.87 | 0.32 | 2.14 | 0.02 | 2.05 | 0.15 |
| INVS | 1.55 | 0.02 | −2.95 | 0.08 | 1.77 | 0.96 | 1.44 | 0.08 |
| DIAPH1 | 2.07 | 0.01 | −1.52 | 0.96 | 2.77 | 0.04 | 1.78 | 0.13 |
| IER3 | 1.51 | 0.04 | 2.15 | 0.02 | 1.55 | 0.43 | 1.35 | 0.36 |
| Q9H640 | 1.77 | 0.02 | 1.48 | 0.37 | −1.99 | 0.21 | −1.97 | 0.10 |
| GBP2 | 1.72 | 0.08 | −1.29 | 0.36 | 1.51 | 0.06 | 1.33 | 0.33 |
| NANS | 1.02 | 0.76 | 1.01 | 0.51 | −1.41 | 0.27 | 1.70 | 0.04 |
| Q86XN7; | 1.67 | 0.20 | 3.71 | 0.04 | 1.08 | 0.41 | 1.78 | 0.14 |
| Q9H9M1 | | | | | | | | |
| TNFAIP3 | 2.50 | 0.14 | 3.45 | 0.02 | 2.34 | 0.04 | 1.20 | 0.67 |
| Q96MJ8; | 1.63 | 0.03 | 1.86 | 0.26 | 1.69 | 0.89 | 2.62 | 0.00 |
| Q9BSE2 | | | | | | | | |
| Q9H753 | 1.15 | 0.21 | 2.32 | 0.00 | 1.12 | 0.77 | 1.31 | 0.24 |
| NTNG1 | 1.55 | 0.11 | 1.29 | 0.27 | 1.09 | 0.53 | 3.39 | 0.06 |
| INHBE | −1.01 | 0.67 | 2.57 | 0.01 | −1.06 | 0.56 | −1.24 | 0.39 |
| BCL6 | 1.02 | 0.22 | 1.95 | 0.01 | 1.20 | 0.48 | 1.20 | 0.81 |
| CXCL1 | 1.93 | 0.12 | 4.56 | 0.03 | 2.08 | 0.63 | 1.09 | 0.49 |
| EHD1 | 1.64 | 0.13 | 3.48 | 0.00 | 1.55 | 0.15 | 1.73 | 0.07 |
| RELB | −1.02 | 0.25 | 2.58 | 0.00 | 2.00 | 0.93 | 1.11 | 0.20 |
| HRK | 3.46 | 0.08 | 2.01 | 1.00 | 2.28 | 0.87 | 2.09 | 0.05 |
| CCL4 | 1.36 | 0.19 | 1.88 | 0.05 | 1.80 | 0.05 | 1.14 | 0.86 |
| SESN2 | −1.05 | 0.88 | 1.30 | 0.16 | 1.62 | 0.01 | 1.12 | 0.45 |
| NAB1 | −1.09 | 0.47 | 2.42 | 0.00 | 1.41 | 0.03 | −1.20 | 0.66 |
| EBI3 | −1.25 | 0.54 | 1.96 | 0.02 | 1.89 | 0.47 | 2.44 | 0.26 |
| DDX21 | 1.21 | 0.37 | 1.55 | 0.00 | 1.60 | 0.01 | 1.31 | 0.05 |
| XBP1 | 1.12 | 0.09 | 1.58 | 0.00 | −1.02 | 0.32 | 1.02 | 0.68 |
| SULRP1; ARS | 2.62 | 0.46 | 1.20 | 0.30 | 1.39 | 0.51 | 1.85 | 0.02 |
| HDAC10 | 1.22 | 0.24 | 1.32 | 0.86 | 1.97 | 0.01 | 1.32 | 0.32 |
| MEP1A | −1.85 | 0.11 | 2.05 | 0.10 | 1.22 | 0.75 | 1.89 | 0.06 |
| RAP2C | 1.27 | 0.29 | 1.54 | 0.03 | 1.31 | 0.50 | 1.08 | 0.22 |
| GYS1 | −1.15 | 0.75 | −1.18 | 0.17 | 1.96 | 0.05 | −1.02 | 0.46 |
| RARRES3 | −1.13 | 0.46 | 1.15 | 0.70 | 1.24 | 0.13 | 2.62 | 0.05 |
| PPY | −4.35 | 0.48 | 2.50 | 0.26 | 1.13 | 0.69 | 5.65 | 0.04 |
| NFKB1 | 1.20 | 0.78 | 1.65 | 0.05 | 1.45 | 0.93 | 1.02 | 0.44 |
| MTL4_HUMAN | −1.26 | 0.87 | 1.52 | 0.01 | 1.18 | 0.08 | 1.03 | 0.41 |
| Q9H040 | −1.19 | 0.89 | −1.26 | 0.52 | 1.51 | 0.00 | −1.53 | 0.22 |
| Q9NUP6 | 1.31 | 0.59 | 1.29 | 0.90 | −1.27 | 0.64 | 1.90 | 0.01 |

TABLE 68
Genes that are upregulated by the Toll-like receptor 4 ligand LPS and
downregulated by LL-37.

| | | | LPS + | |
|---|---|---|---|---|
| | | LPS | LL37 | LL37 |
| | | fold | fold | fold |
| Gene Name | Gene Description | change | change | change |
| LC2A6 | Facilitative glucose transporter; binds cytochalasin B with low affinity | 7.04 | 1.13 | 1.41 |
| SLC4A5 | HCO3-transporter; Na+/HCO3-co-transporter | 6.80 | 1.52 | 4.72 |
| MCL1 | Apoptosis regulator Bcl-2 protein, BH | 6.31 | 1.73 | 1.72 |
| Q86XN7; Q9H9M1 | Aldehyde dehydrogenase; Proline-rich extensin; Proline-rich region | 6.00 | 1.41 | 2.29 |
| Q86UU3; Q8NAA1 | Proline-rich extensin; Proline-rich region | 5.41 | −1.08 | 1.16 |
| C15orf2 | low complexity | 5.24 | −2.56 | −1.29 |
| TNFRSF5 | Receptor for TNFSF5/CD40L | 5.24 | −1.30 | 1.82 |
| FACL6 | Activation of long-chain fatty acids for both synthesis of cellular lipids, and degradation | 5.09 | 1.50 | 2.61 |
| | via beta-oxidation. | | | |
| Q8IW99; Q96AU7 | Thymic Stromal Lymphopoietin Isoform 2. | 4.92 | −1.12 | −1.20 |
| PRB4 | Salivary proline-rich protein II-1 | 4.9 | −1.02 | −1.29 |
| Q9NWP0 | low complexity | 4.89 | −1.20 | −1.06 |
| Q8NF24; Q8TEE5 | β-Ig-H3/Fasciclin domain; Proline-rich extension | 4.60 | 1.45 | 1.06 |
| PDE4DIP | Similar to Rat Myomegalin. | 4.56 | 1.27 | −1.42 |
| NUDT4 | Nudix hydrolase | 4.55 | −1.33 | −1.39 |
| DUSP2 | Regulates mitogenic signal transduction by dephosphorylating both Thr and Tyr | 4.42 | 1.35 | 1.46 |
| | residues on MAP kinases ERK1 and ERK2 | | | |
| LMAN2 | Intracellular lectin in the early secretory pathway; transport and sorting of high | 4.38 | −1.41 | −1.37 |
| | mannose-type glycoproteins | | | |
| RELB | Stimulates promoter activity in the presence of p49- and p50-NFκB. Neither associates | 4.30 | 1.96 | 1.23 |
| | with DNA nor with p65-NFκB | | | |
| SNF1LK | Probable serine/threonine-protein kinase SNF1LK | 4.27 | 1.25 | 1.93 |
| TNFα | Cytokine that binds to TNFRSF1A/TNFR1 and TNFRSF1B/TNFBR. | 4.25 | 1.14 | 2.64 |
| GHRHR | G protein-coupled receptor for growth hormone GRF. | 4.11 | −3.22 | 1.01 |
| TNFSF6 | Cytokine that binds to TNFRSF6/FAS, a receptor that transduces the apoptotic signal | 3.79 | 1.32 | 1.69 |
| | into cells. | | | |
| ENSG00000181873 | Glycine cleavage T protein (aminomethyl transferase) | 3.78 | −1.18 | 1.96 |
| IRAK2 | Required for IL1R-induced NFκB activation. Proximal mediators of IL-1 signaling | 3.71 | 1.41 | 1.46 |
| CKB | Reversibly catalyzes the transfer of phosphate between ATP and various phosphogens | 3.60 | 1.39 | 1.57 |
| | (e.g. creatine phosphate). | | | |
| CASR | Senses changes in the extracellular concentration of calcium ions. | 3.51 | 1.01 | −1.47 |
| KRTAP4-10 | Keratin, high sulfur B2 protein; von Willebrand factor, type C | 3.45 | 1.69 | −3.16 |
| ARHGEF3 | DH domain; Pleckstrin-like | 3.43 | 1.01 | 1.10 |
| CYP3A4; CYP3A7 | P450 Cytochrome. | 3.43 | −4.24 | −1.00 |
| GPR27 | Orphan receptor. Possible candidate for amine-like G-protein coupled receptor | 3.41 | 1.25 | −1.83 |
| PAX8 | Transcription factor for the thyroid-specific expression of the genes. | 3.37 | −1.95 | −5.99 |
| GAP43 | Associated with nerve growth. Major component of the motile & growth cones | 3.36 | 1.87 | −1.81 |
| Q96M75; Q9H568 | Actin/actin-like | 3.31 | −2.73 | 1.50 |
| AGTRL1 | Receptor for apelin coupled to G proteins that inhibit adenylate cyclase activity. | 3.24 | 2.00 | 1.24 |
| | Alternative co-receptor with CD4 for HIV-1 infection. | | | |
| C1orf22 | Putative α-mannosidase C1orf22 | 3.21 | 1.17 | 1.11 |
| EHD1 | EH-domain containing protein 1; Testilin; hPAST1 | 3.20 | 1.58 | 1.6 |
| ADRA1B | G protein-coupled α-adrenergic receptor | 3.17 | 1.62 | −1.60 |
| SSTR2 | G protein-coupled receptor for somatostatins-14 and -28. | 3.17 | 1.09 | 1.27 |
| SYNE1 | Involved in the maintenance of nuclear organization and structural integrity. Connects | 3.16 | 1.37 | −1.30 |
| | nuclei to the cytoskeleton. | | | |
| ENSG00000139977 | Bipartite nuclear localization signal; GCN5-related N-acetyltransferase | 3.15 | −1.94 | −1.20 |
| PTPRK | Regulator of processes involving cell contact and adhesion such as growth control, | 3.13 | 1.33 | 1.19 |
| | tumor invasion, and metastasis. | | | |
| O15059; Q9NZ16 | Guanine-nucleotide dissociation stimulator CDC25; Pleckstrin-like | 3.13 | 1.28 | 3.43 |
| N4BP3; KIAA0341 | Nedd4-binding protein 3; N4BP3 | 3.11 | −1.28 | 1.60 |
| Q8IVT2 | coiled-coil; low complexity | 3.10 | 1.32 | −1.73 |
| Q9NV39 | low complexity | 3.08 | −1.39 | −1.72 |
| HIP1R; HIP12; | Component of clathrin-coated pits and vesicles, may link the endocytic machinery to | 3.06 | −1.22 | 1.21 |
| KIAA0655 | actin cytoskeleton | | | |
| IL-6 | Cytokine with a wide variety of biological functions | 3.04 | 1.11 | 1.46 |
| TNFAIP2 | May play a role as a mediator of inflammation and angiogenesis; Probably function in | 2.97 | 1.54 | 1.0 |
| | nuclear protein import as nuclear transport receptor. | | | |
| RCV1 | Seems to be implicated in the pathway from retinal rod guanylate cyclase to rhodopsin. | 2.95 | −1.38. | −1.69 |
| FBLN2 | Its binding to fibronectin and some other ligands is calcium dependent | 2.95 | 1.14 | −1.04 |
| TWIST2 | Inhibits transcriptional activation by MYOD1, MYOG, MEF2A and MEF2C. Represses | 2.92 | 1.80 | 2.05 |
| | expression of proinflammatory cytokines such as TNFα and IL1β. | | | |
| PARD6B | Adapter protein involved in asymmetrical cell division and polarization processes and | 2.88 | −3.02 | 1.46 |
| | formation of epithelial tight junctions. | | | |
| DCK | Required for the phosphorylation of several deoxyribonucleosides. | 2.84 | 1.23 | 1.65 |
| TULP4 | Tubby-like protein 4; Tubby superfamily protein | 2.83 | −2.18 | 1.07 |
| KLK10 | Has a tumor-suppressor role for NES1 in breast and prostate cancer | 2.81 | 1.40 | 1.25 |
| SPAP1 | Immunoglobulin-like | 2.80 | 1.23 | 2.35 |
| IBRDC2 | Zn-finger, RING; Zn-finger, cysteine-rich C6HC | 2.79 | −1.64 | 1.03 |
| JAM2 | May play a role in the processes of lymphocyte homing to secondary lymphoid organs | 2.77 | −2.6 | −1.44 |
| NRG2 | Direct ligand for ERBB3 and ERBB4 tyrosine kinase receptors. May also promote the | 2.74 | −1.44 | 2.31 |
| | heterodimerization with the EGF receptor | | | |
| CBARA1 | Bipartite nuclear localization signal; Calcium-binding EF-hand | 2.74 | 1.5 | 1.74 |
| DLG2 | Interacts with the cytoplasmic tail of NMDA receptor subunits as well as potassium | 2.66 | 1.55 | −1.0 |
| | channels | | | |
| PRKCBP1 | Protein kinase C binding protein 1 | 2.66 | −3.68 | −1.42 |
| MGLL | Alpha/beta hydrolase; Alpha/beta hydrolase fold; Esterase/lipase/thioesterase, active | 2.65 | 1.56 | 1.07 |
| | site; Lipase | | | |
| Q9BYE1 | Chymotrypsin serine protease, family S1; Low density lipoprotein-receptor, class A; | 2.60 | −2.52 | −3.84 |
| MARCKS | MARCKS is the most prominent cellular substrate for protein kinase C. Binds | 2.60 | 1.33 | 1.13 |
| | calmodulin, actin, and synapsin and is an F-actin cross-linking protein | | | |
| Q96N98 | Amidase | 2.60 | 1.25 | 1.07 |
| Q8NBY1; Q96AF2; | Bipartite nuclear localization signal; Protein kinase; Tyrosine protein kinase | 2.60 | 1.28 | 1.30 |
| Q9BS16 | Soxlz/Sox6-binding protein SolT. | 2.58 | −2.57 | 1.82 |
| PPP2CA | Protein phosphatase PP2A can modulate the activity of MAP-2 kinase and other | 2.58 | −1.47 | 1.19 |
| | kinases. | | | |
| RAB38 | May be involved in melanosomal transport and docking. Involved in the proper sorting | 2.54 | −1.778 | 1.62 |
| | of TYRP1 | | | |
| VCAM1 | Important in cell-cell recognition. VCAM1/VLA4 interaction may play a role in | 2.53 | 1.46 | 2.21 |
| | immune responses and in leukocyte emigration to inflammation sites | | | |
| TTTY8 | Transcript Y 8 protein | 2.52 | 1.22 | −1.13 |
| HTR2A | One of the several different serotonin G protein-coupled receptors | 2.51 | −1.20 | −1.35 |
| SERPINB10 | May play a role in the regulation of protease activities during hematopoiesis | 2.51 | 1.51 | −5.00 |
| O75121; Q9BVE1 | Immunoglobulin-like | 2.51 | −2.15 | −1.07 |
| ZCCHC2 | Phox-like; Zn-finger, CCHC type | 2.50 | −1.04 | 1.60 |
| CXCL2 | Chemokine produced by activated monocytes & neutrophils and expressed at | 2.50 | 1.38 | 1.42 |
| | inflammation sites | | | |
| GADD45B | Involved in the regulation of growth & apoptosis. Mediates activation of | 2.48 | 1.29 | 1.17 |
| | MTK1/MEKK4 MAPKKK | | | |
| KARS | Lysy1-tRNA synthetase LysRS | 2.43 | 1.29 | −2.94 |
| SCG2 | Secretogranin II; a neuroendocrine secretory granule protein, biologically active peptide | 2.42 | −1.83 | 1.45 |
| | precursor | | | |
| SLC17A2 | May be involved in actively transporting phosphate into cells via Na(+) cotransport | 2.41 | 1.03 | 1.08 |
| FLT4 | Receptor for VEGFC. Has a tyrosine-protein kinase activity | 2.41 | 1.41 | 2.48 |
| Q9NXT0 | KRAB box; Zn-finger, C2H2 type | 2.38 | 1.01 | −1.22 |
| Q96L19 | L-lactate dehydrogenase; | 2.38 | 1.00 | 1.12 |
| BICD1 | Drosophila Bicaudal D Homolog 1 | 2.34 | −1.66 | −4.36 |
| HCK | May also contribute to neutrophil migration and may regulate the neutrophil | 2.32 | 1.72 | 1.11 |
| | degranulation | | | |
| Q8N9T8; Q9H978 | Krr1 | 2.31 | −1.26 | −2.64 |
| PPP1R1A | Inhibitor of protein-phosphatase 1. | 2.31 | −3.64 | 1.33 |
| PAX7 | Probable transcription factor. May have a role in myogenesis | 2.31 | −1.01 | 1.52 |
| EBI3 | Cytokine receptor | 2.29 | 1.69 | 2.00 |
| THRA | Nuclear hormone receptor. High affinity receptor for triiodothyronine | 2.29 | −3.93 | −1.63 |
| SLC16A10 | Solute carrier family 16 (Monocarboxylate transporters), member 10 | 2.25 | −1.72 | 6.63 |
| INPP5E | Endonuclease/exonuclease/phosphatase family; Prenyl group binding site (CAAX box) | 2.25 | 1.16 | 2.82 |
| Q9H967 | Bipartite nuclear localization signal; G-protein beta WD-40 repeat | 2.23 | 1.50 | 3.75 |
| NFKB1 | NFκB1 p105 and p50 subunits involved in immune response and acute phase reactions. | 2.21 | 1.36 | 1.09 |
| MKL1 | Antiapoptotic transcriptional factor that acts as a cofactor of serum response factor | 2.21 | 1.24 | −1.08 |
| | (SRF). | | | |
| SS18L2 | SS18-like protein 2; SYT homolog-2 | 2.17 | 1.16 | 1.09 |
| TNFRSF9 | Receptor for TNFSF14/4-1BBL. Possibly active during T cell activation | 2.16 | 1.02 | −1.37 |
| TNFAIP6 | Possibly involved in cell-cell and cell-matrix interactions during inflammation & | 2.16 | 1.55 | −1.17 |
| | tumorgenesis | | | |
| Q9Y2K2 | Protein kinase; Serine/Threonine protein kinase; Tyrosine protein kinase | 2.14 | 1.16 | 1.12 |
| ING5 | Zn-finger-like, PHD finger | 2.11 | 1.77 | 1.12 |
| IL1A | Pro-inflammatory cytokine. | 2.11 | 1.35 | −2.22 |
| TMH | unknown | 2.10 | −1.15 | 1.38 |
| HDAC4 | Histone deacetylase acts on lysine residues on the N-terminus of core histones. | 2.10 | −1.44 | −1.02 |
| KPTN | Kaptin actin-binding protein. | 2.10 | 1.41 | 2.98 |
| SEC61G | Necessary for protein translocation in the endoplasmic reticulum | 2.07 | −1.14 | 4.02 |
| Q9Y484 | G-protein beta WD-40 repeat | 2.07 | 1.08 | −2.49 |
| FRAS1 | von Willebrand factor, type C Cytochrome c heme-binding site; Signal peptidase; | 2.05 | −3.27 | 2.13 |
| IER5 | Immediate early response 5. | 2.01 | −1.06 | 1.37 |
| Q8N137; Q8NCB8 | LysT-interacting protein Lip8. | 2.01 | −1.16 | 2.01 |
| Q96HQ0; Q9H5P0 | ATP/GTP-binding site motif A (P-loop); KRAB box; Zn-finger, C2H2 subtype; | 2.00 | −1.31 | 1.94 |
| TXNRD1 | Thioredoxin reductase, cytoplasmic precursor; TR; TR1 | 1.99 | 1.17 | 1.06 |
| CAV2 | Caveolin-2; May act as a scaffolding protein within caveolar membranes. | 1.98 | −1.17 | −1.48 |
| SCARB1 | CD36 antigen | 1.97 | −1.16 | 2.25 |
| MAP3K5 | Phosphorylates and activates two different subgroups of MAP kinase kinases. | 1.96 | 1.16 | 1.375 |
| PDHX | Required for anchoring dihydrolipoamide dehydrogenase (E3) to pyruvate | 1.96 | 1.32 | 1.23 |
| | dehydrogenase | | | |
| TCEB3 | SIII, or elongin, is a general transcription elongation factor. | 1.95 | 1.07 | 2.51 |
| C21orf55 | May have a role in protein folding or as a chaperone | 1.95 | 1.07 | 2.03 |
| MPHOSPH10 | Component of U3 nucleolar small nuclear ribonucleoprotein. Processing preribosomal | 1.94 | 1.19 | 1.22 |
| | RNA | | | |
| PDE8A | Phosphodiesterase plays a role in signal transduction by regulating the intracellular | 1.93 | −1.33 | 1.17 |
| | concentration of cyclic nucleotides. | | | |
| TFR2 | Transferrin receptor 2. Cellular iron uptake o | 1.92 | −1.57 | 1.60 |
| FARP1 | Band 4.1 domain; DH domain; Pleckstrin-like | 1.92 | 1.26 | 10.39 |
| SERPINA1 | Inhibitor of serine proteases. Primary target is elastase. Moderate affinity for plasmin, | 1.92 | 1.30 | 1.23 |
| | thrombin | | | |
| MYO15A | Myosins-15A; Unconventional myosins serve in intracellular movements. | 1.91 | 1.32 | −1.59 |
| RABGGTA | Catalyzes the transfer of a geranyl-geranyl moiety from geranyl-geranyl pyrophosphate | 1.89 | 1.27 | −1.22 |
| | to both cysteines in certain Rab proteins. | | | |
| KCNMB4 | Calcium-activated BK potassium channel, beta subunit | 1.89 | 1.12 | 1.56 |
| Q9BR02 | Bipartite nuclear localization signal; Ribosomal protein L23, N-terminal domain | 1.89 | −1.08 | 1.54 |
| APOB | Apolipoprotein B; Recognition signal for the cellular binding and internalization of | 1.88 | 1.39 | −1.48 |
| | LDL. | | | |
| MYC | Binds DNA both in a non-specific manner and activates transcription of growth-related | 1.87 | 1.23 | 1.1 |
| | genes | | | |
| FARP2 | Band 4.1 domain; DH domain; Pleckstrin-like | 1.85 | 1.32 | 1.12 |
| TFAP2BL1 | Transcription factor AP-2 | 1.84 | 1.22 | 2.04 |
| Q86U90; Q9H5F8 | SUA5/yciO/yrdC, N-terminal | 1.82 | 1.07 | −1.01 |
| USH1C | May be involved in protein-protein interaction | 1.81 | −1.29 | 1.22 |
| SOX2 | Transcription factor SOX-2 | 1.78 | 1.32 | −1.19 |
| Q9NVC3 | Amino acid/polyamine transporter, family II | 1.78 | −1.57 | 2.57 |
| NEIL2 | Formamidopyrimidine-DNA glycolase | 1.76 | −1.21 | 1.91 |
| TNIP1 | Interacts with TNFAIP3 and inhibits TNF-induced NFκB-dependent gene expression | 1.75 | 1.41 | 1.09 |
| ADRA1D | This alpha-adrenergic receptor mediates its effect through the influx of extracellular | 1.72 | −1.96 | −1.0792 |
| | calcium | | | |
| PCDHB9 | Potential calcium-dependent cell-adhesion protein. | 1.72 | −2.70 | 1.96 |
| Q12987 | Bipartite nuclear localization signal | 1.71 | −1.06 | 1.18 |
| TNFRSF6 | Receptor for TNFSF6/FASL. | 1.71 | 1.49 | 1.75 |
| C20orf72 | Protein C20orf72 | 1.70 | 1.14 | 1.67 |
| DNAJA3 | Modulates apoptotic signal transduction or effector structures within the mitochondrial | 1.69 | −1.20 | −1.26 |
| | matrix. | | | |
| MAB2IL1 | Guanylate kinase; Mab-21 protein | 1.67 | −3.06 | −1.43 |
| BIRC2 | Apoptotic suppressor. Interacts with TRAF1 and TRAF2. | 1.67 | 1.34 | 1.12 |
| MYST1 | MOZ/SAS-like protein | 1.66 | 1.32 | 3.50 |
| CNN3 | Thin filament-associated protein | 1.66 | 1.00 | 1.12 |
| CXCL3 | Chemokine: May play a role in inflammation. | 1.65 | −2.13 | −1.215 |
| CD80; CSRP2; | Involved in the costimulatory signal essential for T lymphocytes activation. | 1.65 | −1.07 | 1.13 |
| RAD51L1 | | | | |
| ADARB1; TNFSF8 | Cytokine that binds to TNFRSF8/CD30. Induces proliferation of T cells; | 1.64 | −1.04 | −3.34 |
| Q8IW74 | unknown | 1.62 | 1.09 | −1.02 |
| UXS1 | NAD-dependent epimerase/dehydratase | 1.62 | 1.11 | −1.04 |
| ENSG00000182364; | Phosphatidylinositol 3- and 4-kinase | 1.61 | −1.46 | −1.19 |
| TNFRSF7 | Receptor for TNFSF7/CD27L. May play a role in survival of activated T-cells. | 1.60 | 1.29 | −1.25 |
| MYBL2 | Transcription factor involved in the regulation of cell survival, proliferation, and | 1.60 | −1.07 | −1.22 |
| | differentiation. | | | |
| RAB33A | Ras-related protein Rab-33A; Small GTP-binding protein S10 | 1.60 | −1.30 | 1.15 |
| ATIC | Bifunctional purine biosynthesis protein PURH; | 1.59 | −1.36 | −1.166 |
| CAMK1 | Phosphorylates synapsin I | 1.59 | 1.26 | 1.53 |
| CCNT1 | Regulatory subunit of the cyclin-dependent kinase pair (CDK9/cyclin T) complex | 1.58 | 1.17 | 1.97 |
| KCNE4 | β subunit of voltage-gated potassium channel complex of pore-forming alpha subunits. | 1.57 | −1.20 | 1.41 |
| BOK | Apoptosis regulator Bcl-2 protein, | 1.56 | −1.21 | 1.12 |
| NF2 | Probably acts as a membrane stabilizing protein | 1.56 | 1.27 | 1.36 |
| PDP2; KIAA1348 | Catalyzes the dephosphorylation/reactivation of the α-subunit of pyruvate | 1.51 | −2.13 | −1.12 |
| | dehydrogenase E1 component | | | |

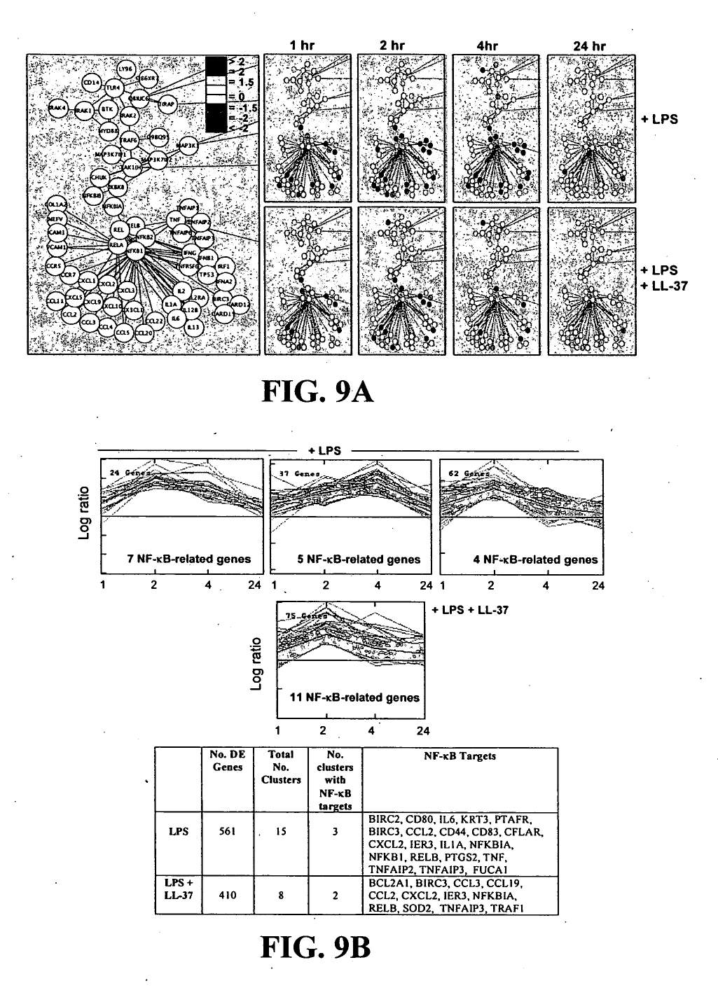

Given that LPS has been known to induce inflammatory responses via the TLR4 to NFκB pathway (Chow JC, et al. Toll-like receptor-4 mediates lipopolysaccharide-induced signal transduction. J Biol Chem 1999; 274:10689-92) and the product of certain differentially expressed genes in the microarray analysis were associated with this pathway, we analyzed in more detail the NFκB-regulated genes and the TLR4 pathway. This pathway was first mapped by integrating protein:protein interaction, signal transduction and regulatory data from the literature into Cytoscape (www.cytoscape.org), an open-source bioinformatics software platform for visualizing molecular interaction networks and integrating these interactions with other data. The microarray expression data was then overlaid onto this signal transduction protein network by colour coding the individual nodes (equivalent to specific genes/proteins) according to the extent of regulation (ranging from red to green, where the intensity of colour demonstrated the extent of up- to down regulation respectively). This then provided a graphic illustration of the genes with altered expression in response to LPS in the absence or presence of SEQ ID NO: 1 at each of the time points (FIG. 9A), and indicated that LPS generally up-regulated genes encoding elements of the TLR4→NFκB pathway, with a peak response at 2-4 hours, and that SEQ ID NO: 1 generally dampened this up-regulation.

To investigate further whether a defined portion of the LPS-responsive genes were likely co-regulated by NFκB, LPS-responsive, differentially-expressed genes with similar temporal expression profiles were clustered using the K-means procedure, a non-hierarchical algorithm, with an affinity threshold of 85% (FIG. 9B). Each cluster thus represented a set of potential co-regulated genes (based on their similar expression profiles over time). Based on this method, the LPS-induced genes were divided into 15 clusters. Three of these clusters, containing a total of 123 genes with peak expression at 2 hr, 4 hr or both, contained 21 genes that are known from the literature to be NFκB-regulated (FIG. 9B). On the other hand, the temporal expression patterns of the 410 genes induced by LPS in the presence of SEQ ID NO: 1 fell into 8 clusters, one of which contained 11 of the 12 differentially expressed NFκB gene targets; six of these NFκB target genes were also included in the subset of LPS-stimulated genes and demonstrated modestly to substantially decreased expression in the presence of SEQ ID NO: 1. Many p50/p65 target genes (Tian B, et al. Identification of direct genomic targets downstream of the NF-kappa B transcription factor mediating TNF signaling. J Biol Chem 2005) were found in the clusters containing the NFκB genes. Thus SEQ ID NO: 1 clearly resulted in the suppression of LPS-stimulation of a substantial number of known NFκB target genes, and clustering data indicated that many other genes that might be NFκB regulated were similarly suppressed. However the data also suggested that the effect observed was selective in that some known NFκB regulated genes were still apparently differentially expressed in the presence of the combination of LPS and SEQ ID NO: 1. To confirm these observations, genes with significant differential expression in response to LPS, and that were differentially affected (remained up-regulated or abrogated) by the presence of the peptide, were selected for validation by quantitative real-time PCR.

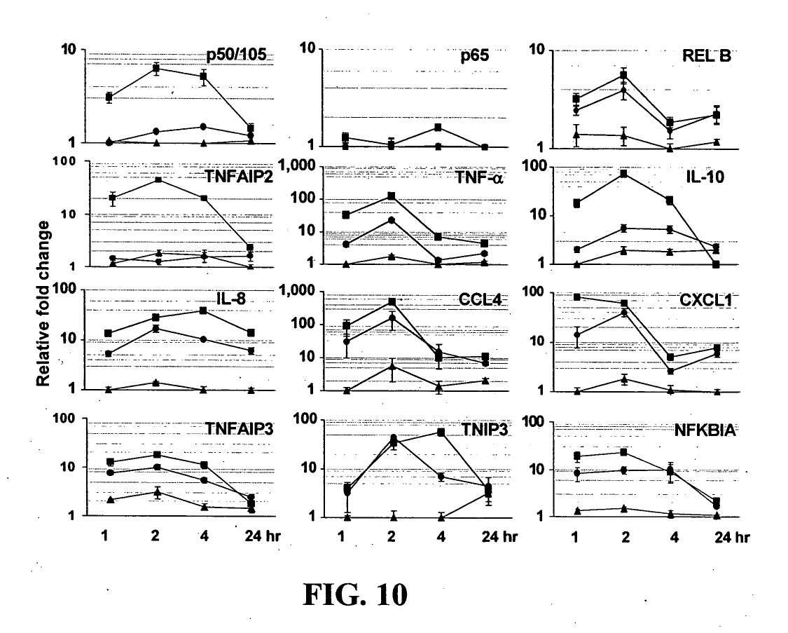

SEQ ID NO: 1 selectively modulates the transcription of specific LPS-induced inflammatory genes. Using qPCR, the expression profiles were validated for 14 of 20 selected genes differentially expressed according to the microarray analysis (FIG. 10). Several known “pro-inflammatory” genes were up-regulated after 2 and 4 hr of treatment with LPS, and this expression level invariably decreased after 24 hr of stimulation. Further, the expression of several LPS-induced genes was confirmed to be altered by the presence of SEQ ID NO: 1. Even though the peptide had a dampening effect on selected LPS-induced expression of inflammatory genes, not all genes up-regulated by LPS were suppressed by the presence of SEQ ID NO: 1, indicating that the effect of SEQ ID NO: 1 on LPS-induced inflammation was selective (FIG. 10). The expression of pro-inflammatory genes such as NFκB1 (p 105/p50) and TNFAIP2 were substantially reduced (90-97%) in LPS-stimulated cells in the presence of SEQ ID NO: 1 at all time points. Also, LPS-induced transcription of TNFα was reduced in the presence of SEQ ID NO: 1 by 87% after 1 hr and around 80% at 2 and 4 hr, but at 24 hr only 58% reduction was observed. Similarly, LPS-induced transcription of IL10 was reduced by more than 90% after 1 and 2 hr in presence of SEQ ID NO: 1, and this effect decreased to 77% after 4 hr. In contrast, the expression of chemoattractants such as IL-8, CCL4, and CXCL1, was slightly reduced by SEQ ID NO: 1 in LPS-stimulated cells but not completely eliminated. Likewise, the expressions of certain anti-inflammatory genes, that are negative regulators of the TLR4 to NFκB pathway were only slightly reduced in the presence of SEQ ID NO: 1. These genes included TNFαIP3 (TNFα-inducible Protein 3) and its interacting partner TNIP3 (TNFαIP3-interacting protein 3), as well as the NFκB-inhibitor, NFκBIA. LPS-induced transcription of NFκB subunit NFκB1 (p105/p50), but not RelB, was completely abrogated by SEQ ID NO: 1, whereas RelA (p65) did not show significant differential expression in response to LPS or SEQ ID NO: 1.

From the temporal transcriptional profiling of LPS-induced genes, it was concluded that SEQ ID NO: 1 did not substantially affect the LPS-induced expression of selected genes that are required for cell recruitment and movement (chemokines) or negative regulators of NFκB. In contrast, SEQ ID NO: 1 neutralized the expression of genes coding for inflammatory cytokines, NFκB1 (p105/p50) and TNFα-induced pro-inflarnmatory genes such as TNFAIP2.

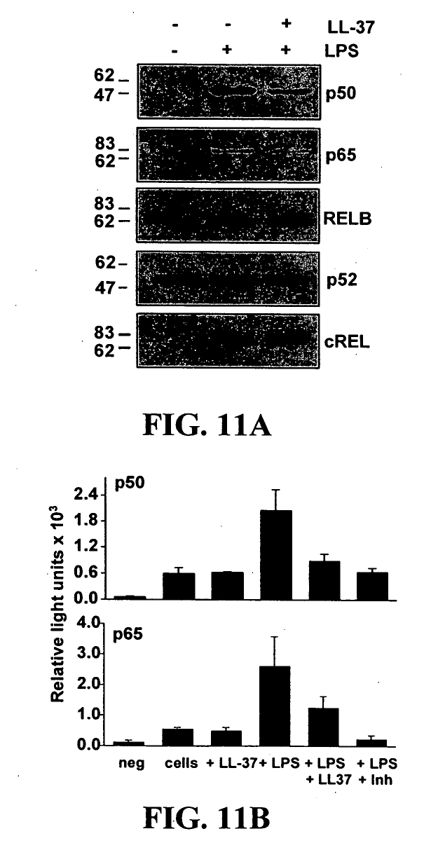

SEQ ID NO: 1 significantly inhibits LPS-induced translocation of the NFκB subunits p50 and p65. The above data indicated that although LL-37 reduced TNFα secretion by more than 95% at all time points, it had a lesser effect (58-87%) in reducing TNFα transcription. To study this in more detail we investigated the key transcription factor NFκB. TLR activation results in nuclear translocation of NFκB, the key transcription factor required for expression of many innate immunity and inflammatory genes (Bonizzi G, et al. The two NF-B activation pathways and their role in innate and adaptive immunity. Trends Immunol 2004; 25:280-8; Li Z W, et al. Genetic dissection of antigen receptor induced-NF-kappaB activation. Mol Immunol 2004; 41:701-14). Although NFκB has a number of subunits with different primary transcriptional regulatory functions, the p50/p65 NFκB heterodimer is most commonly implicated in the regulation of immunity genes. Nevertheless, transcriptionally active NFκB heterodimers other than p50/p65 have important functions as it has been shown that they can influence gene responses to bacterial molecules as well as susceptibility to a variety of infections (Tato CM, et al. Host-Pathogen interactions: Subversion and utilization of the NF-κB pathway during infection. Infect Immunity 2002; 70:3311-7; Mason N, et al. Cutting edge: identification of c-Rel-dependent and -independent pathways of IL-12 production during infectious and inflammatory stimuli. J Immunol 2002;168:25904). To determine if SEQ ID NO: 1 suppressed LPS-induced changes in gene expression by affecting NFκB translocation into the nucleus, the nuclear localization of five NFκB subunits was assessed by Western blots. All monitored subunits of NF-κB (p105/50, p65, c-Rel, Rel B and p100/52) were detected in the nuclear extracts of THP-1 cells (FIG. 11A). The nuclear localization of p50, p65, c-Rel and Rel B, and to a lesser extent p100/52, was increased in THP-1 cells stimulated with LPS for 30 and 60 min (by 60 mins, LPS had induced a 3.5 fold increase in nuclear p5O, a 4.5 fold increase in p65, a 1.7 fold increase in RELB and c-REL, and a 1.2 fold increase in p100/52 as assessed by densitometry). The LPS-induced translocation of p50, p65 and Rel B was clearly suppressed in the presence of SEQ ID NO: 1 as there was around a 35-70% decrease in subunit translocation after 60 min (FIG. 11A), while p100/52 and c-Rel did not appear to be affected.

To more accurately quantify the translocation of p50 or p65, the nuclear extracts were analyzed by ELISA-based immunoassays specific for these subunits (FIG. 11B). SEQ ID NO: 1 suppressed, by slightly more than 50%, LPS-induced p50 and p65 translocation at 30 and 60 min (54±4% and 56±4% inhibition of p50 at 30 and 60 min respectively and 57±8% and 54±3% inhibition of p65 at 30 and 60 min respectively). As a control, it was demonstrated that polymyxin B, a known inhibitor of LPS-LBP (LPS-binding protein) engagement, more substantially inhibited the translocation of NFRB subunits p50 and p65 (82±5% and 80±90% respectively at 60 min; data not shown), demonstrating that TLR4 to NFκB activation can be blocked significantly by agents acting at the cell surface. Although SEQ ID NO: 1 has been reported to activate signal transduction pathways including MAPK in human monocytes and lung epithelial cells (Bowdish D, et al. The human cationic peptide LL-37 induces activation of the extracellular signal-regulated kinase and p38 kinase pathways in primary human monocytes. J Immunol 2004; 172:3758-65), SEQ ID NO: 1 did not promote translocation of NF-κB subunits in human THP-1 cells. Together, these data demonstrate that SEQ ID NO: 1 can moderately alter the LPS-induced translocation of NFκB subunits, thereby providing one mechanism by which SEQ ID NO: 1 suppressed pro-inflammatory cytokine production.

To evaluate the anti-endotoxic activity of SEQ ID NO: 1, two different concentrations of LPS, 10 ng/ml and 100 ng/ml respectively, were used to stimulate human monocytic cells in the presence or absence of this host defense peptide, in an attempt to reflect concentrations of endotoxin ranging from the presumably low concentrations secreted by the normal flora (homeostatic conditions) and early in infection, to those observed in septic infections. To date there has been considerable controversy concerning the role of SEQ ID NO: 1 in human infections, particularly at physiological concentrations. Direct antimicrobial action will certainly occur at low salt concentrations but in the presence of more physiological concentrations of Na+(130 mM) and Mg²⁺/Ca²⁺(1-2 mM) found in tissues and in tissue culture medium (as employed here), SEQ ID NO: 1 has weak or no direct antimicrobial action at the peptide concentrations (1-5 μg/ml) apparently present at mucosal surfaces (Bowdish DM, et al. Impact of SEQ ID NO: 1 on anti-infective immunity. J Leukoc Biol 2005; 77:451-9). Nevertheless there is clear evidence of an anti-infective role (Scott MG, et al. The human antimicrobial peptide LL-37 is a multifunctional modulator of innate immune responses. J Immunol 2002; 169:3883-91; Bowdish DM, et al. Impact of LL-37 on anti-infective immunity. J Leukoc Biol 2005; 77:451-9; Kirikae T, et al. Protective effects of human 18-kilodalton cationic antimicrobial protein (CAP-18)-derived peptide against murine endotoxemia. Infect Immun 1998; 66:1861-8; Fukumoto K, et al. Effect of antibacterial cathelicidin peptide CAP 18/LL-37 on sepsis in neonatal rats. Pediatr Surg Int 2005; 21:20-4; Ciornei CD, et al. Antimicrobial and chemoattractant activity, Lipopolysaccharide neutralization, cytotoxicity, and inhibition by serum of analogs of human cathelicidin LL-37. Antimicrob Agents Chemother 2005; 49:2845-50), which could be explained if SEQ ID NO: 1 has a role in modulating innate immunity. Consistent with this concept, at physiological concentrations SEQ ID NO: 1 is able to mediate chemotaxis (Agerberth B, et al. The human antimicrobial and chemotactic peptides LL-37 and alpha-defensins are expressed by specific lymphocyte and monocyte populations. Blood 2000; 96:3086-93; Yang D, et al. Participation of mammalian defensins and cathelicidins in anti-microbial immunity: receptors and activities of human defensins and cathelicidin (LL-37). J Leukoc Biol 2001; 69:691-7; Niyonsaba F, et al. A cathelicidin family of human antibacterial peptide LL-37 induces mast cell chemotaxis. Immunology 2002;106:20-6), MAP kinase phosphorylation (Scott MG, et al. The human antimicrobial peptide LL-37 is a multiftuctional modulator of innate immune responses. J Immunol 2002; 169:3883-91; Tjabringa G S, et al. The antimicrobial peptide LL-37 activates innate immunity at the airway epithelial surface by transactivation of the epidermal growth factor receptor. J Immunol 2003; 171:6690-6; Bowdish D, et al. The human cationic peptide LL-37 induces activation of the extracellular signal-regulated kinase and p38 kinase pathways in primary human monocytes. J Immunol 2004;. 172:3758-65; Lau YE, et al. Interaction and cellular localization of the human host defense peptide LL-37 with lung epithelial cells. Infect Immun 2005; 73:583-91), Ca²⁺mobilization (Niyonsaba F, et al. Evaluation of the effects of peptide antibiotics human beta-defensins-1/-2 and LL-37 on histamine release and prostaglandin D(2) production from mast cells. Eur J Immunol; 2001; 31:1066-75) and IL-8 release in GM-CSF treated monocytes (Bowdish D M, et al. Impact of LL-37 on anti-infective immunity. J Leukoc Biol 2005; 77:451-9), and as shown herein, anti-endotoxic activity.

The sole human cathelicidin peptide, SEQ ID NO: 1, has been shown to protect animals against endotoxemia/sepsis. Low, physiological concentrations of SEQ ID NO: 1 (≦1 μg/ml) are able to modulate inflammatory responses by inhibiting the release of the pro-inflammatory cytokine TNFα in LPS-stimulated human monocytic cells. Microarray studies established a temporal transcriptional profile, and identified differentially expressed genes in LPS-stimulated monocytes in the presence or absence of SEQ ID NO: 1. SEQ ID NO: 1 significantly inhibited the expression of specific pro-inflammatory genes upregulated by NFκB in the presence of LPS, including NFκB1 (p105/p50) and TNFα-induced protein 2 (TNFAIP2). In contrast, SEQ ID NO: 1 did not significantly inhibit LPS-induced genes that antagonize inflammation, such as TNFα-induced protein 3 (TNFAIP3) and the NFκB inhibitor, NFκBIA, or certain chemokine genes that are classically considered pro-inflammatory. Nuclear translocation, in LPS-treated cells, of the NFκB subunits p50 and p65 was reduced >50% in the presence of SEQ ID NO: 1, demonstrating that the peptide altered gene expression in part by acting directly on the TLR to NFκB pathway. SEQ ID NO: 1 almost completely prevented the release of TNFα and other cytokines by human peripheral blood mononuclear cells (PBMC) following stimulation with LPS and other TLR2/4 and TLR9 agonists, but not with cytokines TNFα or IL1β. Biochemical and inhibitor studies were consistent with a model whereby SEQ ID NO: 1 modulated the inflammatory response to LPS/endotoxin and other agonists of TLRs by a complex mechanism involving multiple points of intervention.

The data presented herein conclusively demonstrates that endotoxin-induced inflammatory gene responses and cytokine secretion in monocytes were suppressed by low, physiological concentrations of SEQ ID NO: 1, implicating SEQ ID NO: 1 in the regulation and control of pro-inflammatory responses associated with pathogenic assault and, by extension, with homeostatic levels of TLR agonists secreted by commensals. The data further demonstrates that SEQ ID NO: 1 can suppress LPS-induced NFκB translocation, and exert an anti-inflammatory effect that is not restricted to endotoxin-induced inflammation. In the human THP-1 monocytic cell line as well as in human PBMC, SEQ ID NO: 1 suppressed pro-inflammatory cytokine production induced by LPS as well as other agonists of TLR2 (LTA, PAM₃CSK4) and in part TLR9 (CpG), but selectively enhanced responses to the pro-inflammatory cytokines IL 1β and TNFα. To gain mechanistic insight, transcriptional responses were profiled using microarrays and real time PCR over the course of 1 to 24 hr to study the effects of SEQ ID NO: 1 on LPS-stimulated monocytes. While the transcription of LPS-induced pro-inflammatory cytokines peaked at 2-4 hr and waned by 24 hr, a single, low dose of SEQ ID NO: 1 suppressed pro-inflammatory cytokine secretion by 1 hr, and this effect was sustained for 24 hr.

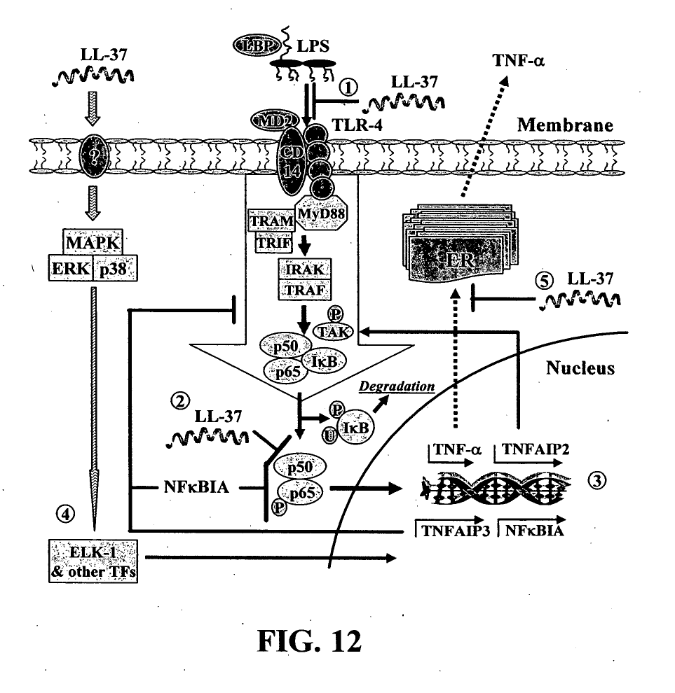

Overall, the data provides evidence that SEQ ID NO: 1 can manipulate both pre- and post-transcriptional events to modulate the TLR-induced inflammatory response in monocytes. A model consistent with the data in this manuscript is outlined in FIG. 12.

LPS-induced activation of NFκB is mediated by TLR4, a receptor containing TIR domain. It is known that receptors with TIR domains are potent activators of NFκB, as well as several other transcription factors such as AP-1, NF-IL6 and IRF3/7 (Takeda K, et al. Toll receptors and pathogen resistance. Cell Microbiol 2003;5:143-53). Mice deficient in TLR4 or MD2 are hyposensitive to LPS, moreover expression of some NFκB target genes is defective without MD2 (Poltorak A, et al. Defective LPS signaling in C3H/HeJ and C57BL/10ScCr mice: mutations in Tlr4 gene. Science 1998; 282:2085-8; Hoshino K, et al. Cutting edge: Toll-like receptor 4 (TLR4)-deficient mice are hyporesponsive to lipopolysaccharide: evidence for TLR4 as the Lps gene product. J Immunol 1999;162:3749-52; Nagai Y, et al. Essential role of MD-2 in LPS responsiveness and TLR4 distribution. Nat Immunol 2002;3:667-72). NFκB is known to play a central role in pathogenesis resulting in sepsis (Brown M A, et al. NF-kappaB action in sepsis: the innate immune system and the heart. Front Biosci 2004; 9:1201-17; Xiao C, et al. NF-kappaB, an evolutionarily conserved mediator of immune and inflammatory responses. Adv Exp Med Biol 2005; 560:41-5) as well as innate immunity to infections (Alcamo E, et al. Targeted mutation of TNF receptor I rescues the RelA-deficient mouse and reveals a critical role for NF-kappa B in leukocyte recruitment. J Immunol 2001; 167:1592-600; Senftleben U, et al. IKKbeta is essential for protecting T cells from TNFalpha-induced apoptosis. Immunity 2001;14:217-30). NFκB transcription factor is a dimeric complex of various subunits that belong to the Rel family; p105/50 (NFκB1), p100/52 (NFκB2), p65 (RelA), RelB, and c-Rel. NFκB proteins share a 300-amino acid Rel homology domain (RHD) that contains a nuclear localization sequence (NLS) and is involved in dimerization, sequence-specific DNA binding and interaction with the inhibitory IkB proteins (Ghosh S, et al. NF-kappa B and Rel proteins: evolutionarily conserved mediators of immune responses. Annu Rev Immunol 1998; 16:225-26). The NFκB proteins form numerous homo- and hetero-dimers that are associated with specific biological responses that stem from their ability to regulate target gene transcription differentially, e.g., p50/p52 dimers function as repressors, whereas Rel A or c-Rel dimers are transcriptional activators. In contrast, RelB does not form homodimers, but instead forms stable heterodimers with either p50 or p52 to exhibit a greater regulatory flexibility, and can be either an activator (Ryseck R P, et al. RelB, a new Rel family transcription activator that can interact with p50-NF-kappa B. Mol Cell Biol 1992; 12:674-84) or a repressor (Ruben S M, et al. I-Rel: a novel rel-related protein that inhibits NF-kappa B transcriptional activity. Genes Dev 1992; 6:745-60). Many inflammatory stimuli trigger signal transduction pathways that result in nuclear localization of NFκB and subsequent transcription of inflammatory and immunity genes encoding for cytokines, chemokines, acute phase reactants, and cell adhesion molecules. The NFκB heterodimer comprising of p50 and p65 subunits has been strongly implicated in transcriptional events triggered by the activation of pro-inflammatory cytokine receptors or TLRs (Ghosh S, et al. NF-kappa B and Rel proteins: evolutionarily conserved mediators of immune responses. Annu Rev Immunol 1998; 16:225-260; Wang T, et al. NF-kappa B and SpI elements are necessary for maximal transcription of toll-like receptor 2 induced by Mycobacterium avium. J Immunol 2001; 167:6924-32). The activation and nuclear translocation of NFκB p50/p65 heterodimer is associated with increased transcription of genes encoding chemokines, cytokines, adhesion molecules such as intercellular adhesion molecule 1 (ICAM-1), vascular cell adhesion molecule-1 (VCAM-1), and endothelial-leukocyte adhesion molecule 1 (ELAM), as well as enzymes that produce secondary inflammatory mediators and inhibitors of apoptosis (Ghosh S, et al. NF-kappa B and Rel proteins: evolutionarily conserved mediators of immune responses. Annu Rev Immunol 1998; 16:225-260). These molecules are important components of the innate immune responses to invading pathogens and are required for migration of inflammatory mediators and phagocytic cells to tissues where NFκB has been activated in response to infection or injury (Pande V, et al. NF-kappaB in human disease: current inhibitors and prospects for de novo structure based design of inhibitors. Curr Med Chem 2005; 12:357-74).

The present invention provides evidence that the host defense peptide, SEQ ID NO: 1, can partially (˜50%) reduce LPS-induced p50/p65 translocation to the nucleus, indicating that this is one mechanism whereby SEQ ID NO: 1 suppressed LPS-induced gene transcription and exerted an anti-endotoxin effect. However if SEQ ID NO: 1 were merely blocking the binding of LPS to the TLR4 receptor through inhibiting its interaction with LBP and/or the LPS receptor complex (Scott M G, et al. Cutting edge: cationic antimicrobial peptides block the binding of lipopolysaccharide (LPS) to LPS binding protein. J Immunol 2000; 164, 549-53), it would be expected that NFκB translocation, and all NFκB-dependent transcriptional events would be inhibited to the same extent as TNFα release, that is>95%; however, this was not observed here. Instead, the effects of SEQ ID NO: 1 on NFκB subunit translocation were selective and relatively modest, and effects on LPS-stimulated transcription of NFκB-regulated genes ranged from very high, e.g., >95% for TNFαIP2 and p105/p50, to moderate (˜80%) for TNFα itself, through to almost no inhibition for other NFκB-regulated genes like TNFAI3. Similarly SEQ ID NO: 1 can protect against sepsis in animal models when administered shortly after endotoxin (Bowdish D, et al. The human cationic peptide LL-37 induces activation of the extracellular signal-regulated kinase and p38 kinase pathways in primary human monocytes. J Immunol 2004; 172:3758-65). In unpublished mouse model experiments (K. Lee, M. G. Scott and R. E. W. Hancock), it was demonstrated that 200 μg of SEQ ID NO: 1 could protect against an 80% lethal dose (400 μg) of E. coli LPS administered peritoneally. Under such circumstances, the LPS would be in 5-fold molar excess and it seems unlikely that in this situation LPS neutralization alone could explain the protection exhibited by SEQ ID NO: 1.

The data presented herein indicates that the host defense peptide SEQ ID NO: 1 can selectively regulate genes that modulate inflammatory responses by suppressing NFκB translocation leading to dysregulation (modulation) of TLR-triggered transcriptional responses. SEQ ID NO: 1 caused inhibition of LPS-triggered pro-inflammatory gene TNFAIP2, but did not neutralize the LPS-induced expression of some of the known negative regulators of NFκB such as TNFAIP3, TNIP3 and NFκBIA (IκBα). Conversely, the transcription of known LPS-induced genes that are regulated by p50/p65 (FIG. 9B) were also inhibited >90% in the presence of SEQ ID NO: 1. However, although NFκB transcription factor activity is influenced by changes in nuclear concentration and subunit composition, the observed ˜50% inhibition of p50/p65 translocation in LPS-induced cells by SEQ ID NO: 1 seems unlikely to completely account for the observed 80% reduction in TNFα gene transcription at 2-4 hr or the >95% reduction in TNFα: protein production and release. Rather, this nearly complete inhibition of pro-inflammatory cytokine release, without an equivalent abrogation of gene transcription, implies that mechanisms other than inhibition of NFκB are also required for SEQ ID NO: 1 to regulate TLR-induced inflammation. Such anomalies demonstrate that SEQ ID NO: 1 influences post-transcriptional events to modulate the inflammatory response. It is therefore shown that SEQ ID NO: 1 affects components of protein translation, maturation or secretion directly and/or indirectly via SEQ ID NO: 1—activated effectors or SEQ ID NO: 1—induced gene transcription (FIG. 12). It is known that SEQ ID NO: 1 can activate components of the MAPK pathway, in particular, p38 (which can influence post-transcriptional events) and ERK, and can promote the activity of the transcription factor, Elk-1 (Bowdish D, et al. The human cationic peptide LL-37 induces activation of the extracellular signal-regulated kinase and p38 kinase pathways in primary human monocytes. J Immunol 2004; 172:3758-65). The putative receptors for SEQ ID NO: 1, including FPRL-1, P2X7, and EGRFR, do not appear to be responsible for SEQ ID NO: 1 induced activation of the MAPK pathway in monocytes (Bowdish D, et al. The human cationic peptide LL-37 induces activation of the extracellular signal-regulated kinase and p38 kinase pathways in primary human monocytes. J Immunol 2004; 172:3758-65). In Drosophila, the LPS or PGN mediated up-regulation of expression of NFκB dependent genes is reported to be suppressed by a MAPK-regulated transcription factor, AP-1 (Kim T, et al. Downregulation of lipopolysaccharide response in drosophila by negative crosstalk between the AP1 and NF-κB signaling modules Nature Immunology 6, 211-218 (2005)). SEQ ID NO: 1 also demonstrates synergy with inflammatory stimuli such as GM-CSF (Bowdish D M, et al. Impact of LL-37 on anti-infective immunity. J Leukoc Biol 2005; 77:451-9; Devine D A, et al. Cationic peptides: distribution and mechanisms of resistance. Curr Pharm Des 2002; 8:703-14) and IL1 P (FIG. 8; Table 66) that likely reflect activation of co-operative signal transduction pathways or transcription of genes whose products contribute to a stabilized, enhanced or prolonged response. Thus, SEQ ID NO: I probably works alone or synergistically with other effector molecules of innate immunity, potentially via the MAPK pathway, to modulate TLR activation and enhance host defense mechanisms.

Accordingly, the data demonstrates that SEQ ID NO: 1 selectively suppresses the pro-inflammatory response in monocytes, particularly the TLR-induced secretion of pro-inflammatory cytokines. The ability of SEQ ID NO: 1 to dampen pro-inflammatory (septic) responses would be valuable for maintaining hormeostasis in the face of natural shedding of microflora-associated TLR agonist molecules, as well as limiting the induction of systemic inflammatory syndrome/septic shock in response to moderate pathogen challenge. The anti-inflammatory effects of SEQ ID NO: 1 were observed at physiologically relevant concentrations of the peptide, and small changes in peptide concentration led to substantial impact on the cellular response to bacterial components such as LPS. SEQ ID NO: 1 thus appears to manifest multiple, complex mechanisms of action, including direct and indirect inhibition of TLR activation and transcription. The improved understanding of the mechanism(s) utilized by SEQ ID NO: 1 to selectively modulate inflammation, and thereby balance the TLR response to commensal or pathogenic bacteria indicates that endogenous cationic host defense peptides are important players in limiting over-active inflammation.

### EXAMPLE 14

### Analysis of Transcriptional Responses Elicited by Synthetic Host Defense Peptides

Endogenous host defense peptides are widely distributed in nature, are essentially amphipathic in nature, 12-50 amino acids in length with a net positive charge of +2 to +7 and are about 50% hydrophobic (Hancock, et al. 1999. Peptide antibiotics. Antimicrob Agents Chemother. 43:1317-1323). As shown above, certain natural host defense peptides expressed in mammalian epithelial and a variety of myeloid cells have been demonstrated to be selectively anti-inflammatory, and are able to maintain the expression of genes that are key players in innate immunity such as certain chemokines, as well as others that antagonize inflammation. In these activities, the peptides are able to act synergistically with other immune mediators such as GM-CSF (FIG. 4) and IL1β. The human peptide LL-37 (SEQ ID NO: 1) induces activation of the extracellular signal-regulated kinase and p38 kinase pathways in primary human monocytes. (FIGS. 3 and 4). Some of these peptides also have the potential to exert overall anti-inflammatory responses, but others have specific therapeutic deficits including the ability to induce apoptosis in certain cells and the stimulation of histamine release through degranulation of mast cells. However, the specificity and efficacy of host defense peptides that can selectively modulate innate immune responses can be improved by deriving synthetic peptides based on the motifs of natural host defense peptides using both random as well as rational design (Hilpert, et al. 2005. High-throughput generation of small antibacterial peptides with improved activity. Nat Biotechnol. 23:1008-1012). Therefore, this study investigated global transcriptional-responses elicited by the synthetic host defense peptide KSRIVPAIPVSLL (SEQ ID NO: 7) in order to unravel the signaling pathways activated by the peptides themselves. The objective was to evaluate the effects of host defense peptides on innate immunity, thereby permitting elucidation of the mechanisms of action as well as biomarkers for peptide action.

The overall effects of synthetic peptide SEQ ID NO: 7 on CD 14⁺monocytes isolated from human peripheral blood mononuclear cells (PBMC) were studied by using a functional genomics and bioinformatics approach. The global transcriptional responses elicited by the peptide, and its effect on bacterial endotoxin-stimulated cells were elucidated in human monocytic cells. Gene profiling technology using DNA microarrays, followed by other transcriptional analysis, e.g., Real-time PCR, and functional analyses applied by the investigators have provided new insight into the molecular events underlying the mechanism elicited by synthetic cationic host defense peptides.

Materials and Methods

The venous blood from healthy volunteers was collected in Vacutainere collection tubes containing sodium heparin as an anticoagulant (Becton Dickinson, Mississauga, ON). White blood cells were isolated from-the buffy coat, and the PBMC was seeded maintained in physiologically relevant RPMI-1640 media (Gibco®, Invitrogen™ Life technologies, Burlington, ON), supplemented with 10% (v/v) heat inactivated fetal bovine serum (FBS), 2 mM L-glutamine and 1 mM sodium pyruvate (all from Invitrogen Life Technologies) at 37° C. in a humidified 5% (v/v) C0₂ incubator. Human monocytic cells, THP-1 (ATCC TIB-202) were grown in suspension in RPMI-1640 complete media as described above. They were treated with 0.3 μg/ml phorbol 12-myristate 13-acetate (PMA; Sigma-Aldrich Canada, Oakville ON) for 24 hr, inducing plastic-adherent cells that were further rested in complete RPMI-1640 medium for an additional 24 hr prior to stimulations with various treatments.

The cells were stimulated with synthetic host defense peptide SEQ ID NO: 7 (200 μg/ml), as well as purified bacterial LPS/endotoxin (2 ng/ml) in the presence and absence of synthetic peptide SEQ ID NO: 7 for 4 hours. The cells were treated with the synthetic peptide 45 mins prior to stimulation with bacterial LPS. Highly purified LPS free of proteins and lipids was obtained from P. aeruginosa strain H103 using the Darveau-Hancock method as previously described (Darveau, et al. 1983. Procedure for isolation of bacterial lipopolysaccharides from both smooth and rough Pseudomonas aeruginosa, Salmonella enterica ssp., and Typhimurium strains. J Bacteriol. 155: 831-838). The synthetic peptide SEQ IID NO: 7 was synthesized using F-moc chemistry.

Following incubation of the cells, the tissue culture supernatants were centrifuged at 1000×g for 5 min, then at 10,000×g for 2 min to obtain cell-free samples, aliquoted and stored at −20° C. prior to assay for various cytokines. Protein secretion were detected with a capture ELISA (eBioscience and BioSource International Inc., CA, USA respectively) using the tissue culture supernatants. In addition, CD14+ monocytes were isolated from the stimulated PBMC after incubation using magnetic Dynal® bead-based separation technology (Invitrogen™). RNA was isolated from the purified monocytes using RNeasy Mini kit, treated with RNase-Free DNase (Qiagen Inc., Canada) and eluted in RNase-free water (Ambion Inc., Austin, Tex., USA) as per the manufacturer's instructions. RNA concentration, integrity and purity were assessed by Agilent 2100 Bioanalyzer using RNA 6000 Nano kits (Agilent Technologies, USA). RNA was (reverse) transcribed with incorporation of amino-allyl-UTP (aa-UTP) using the MessageAmpII™ amplification kit, column purified and eluted in nuclease-free water, and labeled with mono-functional dyes, Cyanine-3 and Cyanine-5 (Amersham Biosciences), according to manufacturer's instructions. Yield and fluorophore incorporation was measured using Lambda 35 UV/VIS fluorimeter (PerkinElmer Life and Analytical Sciences, Inc., USA).

Microarray slides were printed with the human genome 21K Array-Ready Oligo Set™ (Qiagen Inc., USA) at The Jack Bell Research Center (Vancouver, BC, Canada). The slides were pre-hybridized and scanned as described (Mookherjee, et al. 2006. Modulation of the TLR-Mediated Inflammatory Response by the Endogenous Human Host Defense Peptide LL-37. J Immunol. 176: 2455-2464). Assessment of slide quality, normalization, detection of differential gene expression and statistical analysis was carried out with ArrayPipe (version 1.6), a web-based, semi-automated software specifically designed for processing of microarray data (www.pathogenomics.ca/arraypipe). Differentially expressed and statistically significant genes were selected from the miocroarray analysis as genes that induced an absolute fold change of at least 1.5 with a p-student value of ≦0.06.

Differential gene expression identified by microarray analysis was validated using quantitative real-time PCR (qPCR) using SuperScript™ III Platinum® Two-Step qRT-PCR Kit with SYBR® Green (Invitrogen Life Technologies), as described (Mookherjee, et al.). Fold changes were calculated after normalization to endogenous human GAPDH and using the comparative Ct method (Pfaffl, M. W. 2001. A new mathematical model for relative quantification in real-time RT-PCR. Nucleic Acids Res. 29: No. 9 e45).

Results and Discussion

Gene profiling using human 21K human DNA microarrays revealed that there were 566 genes differentially expressed in human monocytes in presence of SEQ ID NO: 7 (Table 69). Of these genes those that were significantly up-regulated by the peptide included G-coupled protein receptors, other transmembrane receptors and co-activators, genes associated with the plasma membrane including integrin, adhesion molecules such as ICAM, NCAM, genes that regulate various tyrosine-protein kinases, critical transcription factors that mediate key pathways in immune responses such as MAPK, JAK-STAT and NFκB. The peptide also up-regulated the expression of various chemotatic factors that play a central role in attracting immune cells to the site of infection and/or trauma. Other significant genes induced by the peptide were those encoding transport proteins including those involved in metal transport, several zinc finger proteins fimctioning as transcription factors, and several genes that are attributed to anti-viral activity.

The genes that were significantly upregulated by the peptide included:
(a) G-coupled protein receptors that initiate signaling from extracellular ligands—representative genes include, but are not limited to, GPR55, GPR6, GPR30, GPCR42, CASR, EDG2;
(b) Chemokines and Interleukins that attract immune cells—representative genes include, but are not limited to, MCP-1, MCP-3, IL-8, IL-17C, as well as receptors for chemokines, e.g., CCR7;
(c) Transcription factors that mediate selective gene expression—representative genes include, but are not limited to, JAK1, STAT1, ELF1, Q9Y4C1, ETV4, POU1F1, Zinc finger proteins e.g., ZNF254, ZNF292, ZNF78L1, Homeobox transcription factor e.g., HOXD3, DLX5;
(d) Tyrosine-protein kinase and their receptors—representative genes include, but are not limited to MAP2K6, NTRK3, PLCG1, EFNA2, NCK1;
(e) Adhesion molecules that mediate cell attachment and interaction—representative genes include, but are not limited to, ICAM, NCAM, as well as cell adhesion receptors e.g., PTPRF;
(f) Genes involved in actin polymerization and cytoskeletal remodelling involved in cell movement and differentiation—representative genes include, but are not limited to, Integrin-α, EPHA4, ARHGAP6, and DST.
(g) Regulators of transcription facotors—representative genes include, but are not limited to, TRIP4, GMEB2, GSK3B, ARNT, BACH1, ARID3A, HIPK2, POLR2D, TGIF, SSBP3, FYB;
(h) Transmembrane receptors and adapters of signaling pathways including, but not limited to WNT5B, receptor for WNT proteins FZD10, TIRAP (adapter for TLR4 pathway), REPS1; and

(i) Genes involved in antiviral activity—representative genes include, but are not limited to Interferons e.g., IFNA2, STAT1 that activates gamma interferon, transcriptional activator MNDA, Interferons that exhibit anti-viral activities-representative genes include, but are not limited to IFNA2.

TABLE 69
Gene profiling using human 21K human DNA microarrays revealing that there
were 566 genes differentially expressed in human monocytes in presence of SEQ ID NO: 7

| | | Fold Change | |
|---|---|---|---|
| Gene Name | Gene Description | SEQ ID NO: 7 | P-value |
| O43300 | Leucine-rich repeat | 80.0 | 0.05 |
| TGM4 | Associated with mammalian reproductive | 71.9 | 0.04 |
| | process. Catalyzes the cross-linking of | | |
| | proteins and the conjugation of polyamines | | |
| | to specific proteins in the seminal tract | | |
| TMOD4 | Blocks the elongation/depolymerization of | 68.0 | 0.04 |
| | the actin filaments at the pointed end. | | |
| Q86Y93 | ATP/GTP-binding site motif A (P-loop); | 64.3 | 0.05 |
| | CSL zinc finger; Heat shock protein DnaJ | | |
| RBP1 | Intracellular transport of retinol | 63.3 | 0.04 |
| Q9C098 | Protein kinase; Serine/Threonine protein | 59.0 | 0.05 |
| | kinase | | |
| Q8NI35 | ATP/GTP-binding site motif A (P-loop); | 57.2 | 0.05 |
| | PDZ/DHR/GLGF domain | | |
| Q8WUC6 | Bipartite nuclear localization signal; Class I | 46.0 | 0.05 |
| | peptide chain release factor domain | | |
| ARNT | Required for activity of the Ah (dioxin) | 45.2 | 0.04 |
| | receptor. This protein is required for the | | |
| | ligand-binding subunit to translocate from | | |
| | the cytosol to the nucleus after ligand | | |
| | binding. This complex then initiates | | |
| | transcription of genes involved in the | | |
| | activation of PAH procarcinogens | | |
| TNC | SAM (substrate-adhesion molecule) that | 45.1 | 0.05 |
| | appears to inhibit cell migration. May play | | |
| | a role in supporting the growth of epithelial | | |
| | tumors. Ligand for integrins α-8/β-1, α- | | |
| | 9/β-1, α-V/β-3 and α-V/β-6 | | |
| POU1F1 | Transcription factor involved in the | 44.5 | 0.04 |
| | specification of the lactotrope, | | |
| | somatotrope, and thyrotrope phenotypes in | | |
| | the developing anterior pituitary. Activates | | |
| | growth hormone and prolactin genes. | | |
| FEZ2 | Involved in axonal outgrowth and | 40.2 | 0.02 |
| | fasciculation | | |
| SMURF2 | E3 ubiquitin-protein ligase which accepts | 38.6 | 0.06 |
| | ubiquitin from an E2 ubiquitin-conjugating | | |
| | enzyme in the form of a thioester and then | | |
| | directly transfers the ubiquitin to targeted | | |
| | substrates, SMAD1, SMAD2 and SMAD7 | | |
| KCNK6 | Exhibits outward rectification in physiological | 36.8 | 0.04 |
| | K(+) gradient & mild inward | | |
| | rectification in symmetrical K(+) | | |
| | conditions | | |
| SMF | SMF protein | 36.8 | 0.04 |
| CNTN5 | Fibronectin, type III; Immunoglobulin-like | 33.6 | 0.05 |
| O43348 | Argininosuccinate synthase | 33.2 | 0.03 |
| WBSCR18 | Williams-Beuren syndrome chromosome | 33.0 | 0.05 |
| | region 18 protein | | |
| Q9Y4T9 | low complexity | 31.7 | 0.05 |
| ZNF595 | Maybe transcriptional repressor. Candidate | 31.5 | 0.05 |
| | gene for Wolf-Hirschhorn (4p-) syndrome | | |
| CD226 | Immunoglobulin-like | 31.0 | 0.04 |
| BNIP1 | Implicated in the suppression of cell death. | 29.7 | 0.05 |
| | Interacts with the BCL-2 and adenovirus | | |
| | E1B 19 kDa proteins | | |
| IDUA | Alpha-L-iduronidase precursor | 29.2 | 0.06 |
| Q8N6Q6 | unknown | 27.1 | 0.05 |
| IL17C | Stimulates the release of TNFα and IL-1β | 26.5 | 0.05 |
| | from the monocytic cell line THP- | | |
| | 1monocytic cell line THP-1 | | |
| RRM2 | Catalyzes the biosynthesis of deoxyribo- | 24.6 | 0.04 |
| | nucleotides from the corresponding | | |
| | ribonucleotides for DNA synthesis | | |
| NR2F2 | Regulation of the apolipoprotein A-I gene | 24.2 | 0.03 |
| | transcription. Binds to DNA site A | | |
| DLX5 | Homeobox protein DLX-5 | 23.4 | 0.04 |
| CHRNA1 | After binding acetylcholine, the AChR | 23.3 | 0.03 |
| | responds by opening of an ion-conducting | | |
| | channel across the plasma membrane | | |
| SLC35A4 | Nucleotide-sugar transporter | 23.2 | 0.05 |
| TRPM8 | Ion transport protein | 22.6 | 0.04 |
| RNF130 | Basic helix-loop-helix dimerization domain | 22.5 | 0.05 |
| | bHLH; Bipartite nuclear localization | | |
| | signal; Protease-associated PA; Zn-finger, | | |
| | RING | | |
| DKC1 | Required for ribosome biogenesis and | 21.8 | 0.05 |
| | telomere maintenance. Probable catalytic | | |
| | subunit of H/ACA small nucleolar | | |
| | ribonucleoprotein complex, which | | |
| | catalyzes pseudouridylation of rRNA. | | |
| LRMP | Lymphoid-restricted membrane protein. | 21.5 | 0.06 |
| HOOK2 | Probable cytoskeletal linker protein, which | 21.4 | 0.03 |
| | may be involved in tethering membrane | | |
| | bound organelles to the cytoskeleton | | |
| DHX8 | Facilitates nuclear export of spliced mRNA | 21.2 | 0.04 |
| | by releasing the RNA from the spliceosome | | |
| Q86WW9 | ATP/GTP-binding site motif A (P-loop); | 20.8 | 0.06 |
| | Lipoxygenase, LH2 domain | | |
| Q9P278 | low complexity | 20.2 | 0.04 |
| STATH | Salivary protein that stabilizes saliva | 20.2 | 0.06 |
| | supersaturated with Ca²⁺ salts; modulates | | |
| | hydroxyapatite crystal formation in teeth | | |
| Q96LW2 | Blue (type 1) copper domain; Protein | 20.1 | 0.05 |
| | kinase; Serine/Threonine protein kinase | | |
| SBNO1 | Helicase, C-terminal; RNA-binding region | 19.7 | 0.05 |
| | RNP-1 (RNA recognition motif) | | |
| IDH3G | Isocitrate dehydrogenase [NAD]subunit | 19.4 | 0.02 |
| | gamma, mitochondrial precursor; Isocitric | | |
| | dehydrogenase; NAD⁺-specific ICDH | | |
| Q9H6R7 | coiled-coil; low complexity | 19.0 | 0.04 |
| PLCG1 | phospholipase C-γ is a major substrate for | 18.4 | 0.05 |
| | heparin-binding growth factor 1-activated | | |
| | tyrosine kinase | | |
| KIAA1529 | coiled-coil; low complexity; | 18.1 | 0.04 |
| | transmembrane | | |
| Q8NHU6 | Bipartite nuclear localization signal; | 17.7 | 0.04 |
| | Maternal tudor protein | | |
| RCL1 | Plays a role in 40S-ribosomal-subunit | 17.4 | 0.01 |
| | biogenesis in the early pre-rRNA | | |
| | processing steps at sites A0, A1 and A2: | | |
| | required for proper maturation of 18S RNA | | |
| Q96HJ9 | unknown | 16.9 | 0.05 |
| FHL3 | Four and a half LIM domains protein 3; | 15.2 | 0.04 |
| | FHL-3; Skeletal muscle LIM-protein 2; | | |
| | SLIM 2 | | |
| Q8N8U9 | Trypsin inhibitor-like, cysteine-rich TIL | 14.8 | 0.03 |
| | region; von Willebrand factor, type C & D | | |
| O60384 | Zn-finger, C2H2 type | 14.8 | 0.05 |
| ENSG00000105849 | RNA polymerase Rpa43 subunit | 14.8 | 0.05 |
| MCART1 | Mitochondrial carrier triple repeat 1 | 14.6 | 0.05 |
| BCL2 | Suppresses apoptosis in a variety of cell | 14.1 | 0.04 |
| | systems. Regulates cell death | | |
| EFNA2 | Ephrin-A2 precursor; EPH-related receptor | 14.1 | 0.04 |
| | tyrosine kinase ligand 6; LERK-6; | | |
| Q9H697 | limkain beta 2. | 13.9 | 0.04 |
| PTGER3 | Receptor for prostaglandin E2 (PGE2) | 13.3 | 0.05 |
| CKMT1 | Reversibly catalyzes the transfer of | 13.1 | 0.05 |
| | phosphate between ATP and various | | |
| | phosphogens (e.g. creatine phosphate). | | |
| TUBB4Q | Tubulin; the major constituent of | 13.1 | 0.06 |
| | microtubules. | | |
| Q7Z483 | Zn-finger, RING | 12.8 | 0.04 |
| DUSP14 | Involved in the inactivation of MAP | 12.7 | 0.05 |
| | kinases. Dephosphorylates ERK, JNK and | | |
| | p38 MAP-kinases | | |
| CYLC2 | Acc: Q14093]; Cylicin II (Multiple-band | 12.5 | 0.03 |
| | polypeptide II). [Source: SWISSPROT | | |
| OR5P2 | Putative odorant receptor. Could also be | 12.2 | 0.04 |
| | involved in taste perception | | |
| EPB41L4B | Band 4.1-like protein 4B; EHM2 protein; | 11.8 | 0.02 |
| | FERM-containing protein CG1 | | |
| IL17B | Stimulates the release of tumor necrosis | 11.6 | 0.03 |
| | factor alpha and IL-1 beta from the | | |
| | monocytic cell line THP-1 | | |
| VPS4A | AAA ATPase, central region; ATP/GTP- | 11.5 | 0.04 |
| | binding site motif A (P-loop); MIT | | |
| C6orf74 | Metal-dependent phosphohydrolase, HD | 11.3 | 0.06 |
| | region | | |
| SYNE1 | Involved in the maintenance of nuclear | 11.3 | 0.06 |
| | organization and structural integrity. | | |
| | Probable anchoring protein which theters | | |
| | the nucleus to the cytoskeleton. | | |
| ZNF322B | Zn-finger, C2H2 type | 10.9 | 0.04 |
| SACM1L | Synaptojanin, N-terminal | 10.9 | 0.04 |
| PRO0461 | Protein PRO0461 | 10.7 | 0.01 |
| ALOX5 | Arachidonate 5-lipoxygenase | 10.6 | 0.04 |
| Q7RTU0 | Basic helix-loop-helix dimerization domain | 10.4 | 0.05 |
| | bHLH | | |
| TRPM3 | Calcium channel mediating constitutive | 10.1 | 0.03 |
| | calcium ion entry. | | |
| POMT2 | Transfers mannosyl residues to the | 9.9 | 0.06 |
| | hydroxyl group of serine or threonine | | |
| | residues. | | |
| PCSK5 | Likely widespread endoprotease activity | 9.9 | 0.04 |
| | within the constitutive and regulated | | |
| | secretory pathway. Cleaves RX(K/R)R | | |
| MAP3K1 | Component of protein kinase signal | 9.8 | 0.05 |
| | transduction cascade. Activates the ERK | | |
| | and JNK kinase pathwaysas well as CHUK | | |
| | and IKBKB, the central protein kinases of | | |
| | the NFκ-B pathway | | |
| BLZF1 | Basic leucine zipper nuclear factor 1. | 9.8 | 0.06 |
| Q96D46 | Cytochrome c heme-binding site; KRAB | 9.6 | 0.03 |
| | box; NMD3 family | | |
| Q9NW81 | Leucine-rich repeat | 9.4 | 0.05 |
| Q9BUJ0 | Alpha/beta hydrolase; | 9.2 | 0.03 |
| | Esterase/lipase/thioesterase, active site | | |
| Q9H5P1 | Zn-finger, C-x8-C-x5-C-x3-H type | 9.1 | 0.05 |
| UBE2N | The UBE2V2/UBE2N heterodimer | 9.1 | 0.04 |
| | catalyzes the synthesis of non-canonical | | |
| | poly-ubiquitin chains that are linked | | |
| | through Lys-63. This type of poly- | | |
| | ubiquitination does not lead to protein | | |
| | degradation by the proteasome. Mediates | | |
| | transcriptional activation of target genes. | | |
| | Plays a role in the control of progress | | |
| | through the cell cycle and differentiation. | | |
| | Plays a role in the error-free DNA repair | | |
| | pathway and contributes to the survival of | | |
| | cells after DNA damage | | |
| O75872 | rab3 GTPase-activating protein, non- | 8.8 | 0.03 |
| | catalytic subunit. | | |
| ANKRD5 | Ankyrin repeat domain protein 5 | 8.6 | 0.05 |
| MAN2B2 | Epididymis-specific alpha-mannosidase | 8.3 | 0.04 |
| | pre-cursor; Mannosidase alpha class 2B | | |
| | member 2 | | |
| RPS7 | 40S ribosomal protein S7; 40S ribosomal | 8.3 | 0.05 |
| | protein S7; S8 | | |
| PDE7B | May be involved in the control of cAMP- | 8.3 | 0.03 |
| | mediated neural activity and cAMP | | |
| | metabolism in the brain | | |
| BACH2 | Component of the dystrophin-glycoprotein | 8.2 | 0.04 |
| | complex (DGC), a complex that spans the | | |
| | muscle plasma membrane and forms a link | | |
| | between the F-actin cytoskeleton and the | | |
| | extracellular matrix. Probably involved in | | |
| | the control of the cell cycle. | | |
| STUB1 | TPR repeat; Zn-finger, modified RING | 8.0 | 0.06 |
| TRIP4 | Transcription coactivator of nuclear | 7.6 | 0.02 |
| | receptors; plays a pivotal role in the | | |
| | transactivation of NF-kappa-B, SRF, AP1. | | |
| WNT5B | Ligand for members of the frizzled family | 7.5 | 0.04 |
| | of seven transmembrane receptors. | | |
| | Probable developmental protein. | | |
| TXNL | Thioredoxin-like protein 1; 32 kDa | 7.5 | 0.03 |
| | thioredoxin-related protein | | |
| ARRB1 | Beta-arrestin: regulates beta-adrenergic | 7.3 | 0.06 |
| | receptor function. | | |
| HNRPH2 | Component of the heterogenous nuclear | 7.3 | 0.04 |
| | ribonucleoprotein (hnRNP) complex | | |
| GSTZ1 | Bifunctional enzyme with minimal | 7.2 | 0.05 |
| | glutathione-conjugating activity and low | | |
| | glutathione peroxidase activity | | |
| PKP4 | May play a role in junctional plaques | 7.1 | 0.05 |
| CD84 | leukocyte antigen CD84 (leukocyte | 7.0 | 0.05 |
| | antigen) | | |
| TUBGCP6 | Gamma-tubulin complex is necessary for | 6.9 | 0.02 |
| | microtublule nucleation at the centrosome | | |
| Q9NUU6 | transmembrane | 6.9 | 0.05 |
| VAMP5 | May participate in trafficking events that | 6.8 | 0.05 |
| | are associated with myogenesis | | |
| RNF41 | Zn-finger, RING | 6.7 | 0.06 |
| ENSG00000178042 | ATP/GTP-binding site motif A (P-loop); | 6.7 | 0.04 |
| | Zn-finger, C2H2 subtype | | |
| TSPAN2 | May play a role in signaling in | | |
| | oligodendrocytes in the early stages of their | 6.6 | 0.04 |
| | terminal differentiation | | |
| Q96E44 | Beta and gamma crystallin; Nuclear protein | 6.5 | 0.03 |
| | SET | | |
| Q8NHE2 | SF21 protein | 6.4 | 0.05 |
| PIP5K1B | Phosphatidylinositol-4-phosphate 5-kinase | 6.4 | 0.03 |
| U2AF1L2 | U2 small nuclear ribonucleoprotein | 6.3 | 0.06 |
| | auxiliary factor 35 kDa subunit related- | | |
| | protein 1 | | |
| EIF2C4 | Plays an important role in the eukaryotic | 6.3 | 0.03 |
| | peptide chain initiation process | | |
| DACH2 | Bipartite nuclear localization signal; | 6.2 | 0.02 |
| | Transforming protein Ski | | |
| Q7Z620 | C2 domain | 6.2 | 0.06 |
| Q96IZ9 | PTD016 protein. | 6.0 | 0.04 |
| IFNA2 | Interferon-alpha; produced by | 6.0 | 0.04 |
| | macrophages, with antiviral activities. | | |
| CCS | Delivers copper to copper zinc superoxide | 6.0 | 0.04 |
| | dismutase (SOD1) | | |
| Q8TDS9 | putative G-protein coupled receptor | 6.0 | 0.03 |
| | GPCR42. | | |
| SPG6 | WW/Rsp5/WWP domain | 5.8 | 0.05 |
| TIRAP | Adapter involved in the TLR4 signaling | 5.7 | 0.04 |
| | pathway in the innate immune response. | | |
| | Acts via IRAK2 and TRAF-6, leading to | | |
| | the activation of NF-kappa-B, MAPK1, | | |
| | MAPK3 and INK, resulting in cytokine | | |
| | secretion and the inflammatory response | | |
| Q96MA7 | coiled-coil; low complexity | 5.7 | 0.06 |
| UBXD2 | UBX domain-containing protein 2 | 5.6 | 0.04 |
| ANKMY1 | Ankyrin repeat and MYND domain protein | 5.6 | 0.01 |
| | 1; Testis-specific ankyrin-like protein 1; | | |
| PCCB | Propionyl-CoA carboxylase beta chain, | 5.6 | 0.05 |
| | mitochondrial precursor; | | |
| TNFSF5IP1 | Hepatocellular carcinoma susceptibility | 5.6 | 0.05 |
| | protein; x 003 protein; TNF superfamily, | | |
| | member 5-induced protein 1 | | |
| PAK2 | The activated kinase phosphorylates | 5.5 | 0.05 |
| | ribosomal protein S6, histone H4 and | | |
| | myelin basic protein | | |
| Wdr68 | WD-repeat protein 68; WD-repeat protein | 5.4 | 0.03 |
| | An11 homolog | | |
| SMYD3 | Histone methyltransferase. Important role | 5.3 | 0.04 |
| | in transcriptional activation | | |
| CENTB2 | GTPase-activating protein for the ADP | 5.3 | 0.02 |
| | ribosylation factor family | | |
| TIMM9 | Likely involved in the import and insertion | 5.2 | 0.05 |
| | of hydrophobic membrane proteins into the | | |
| | mitochondrial inner membrane | | |
| Q9P1G1 | signal peptide | 5.2 | 0.05 |
| C21orf108 | Nucleolar preribosomal-assoc. protein 1 | 5.1 | 0.04 |
| FBXW5 | Cyclin-like F-box; G-protein beta WD-40 | 5.0 | 0.02 |
| | repeat | | |
| ARHGAP20 | RA domain; RhoGAP domain | 4.9 | 0.04 |
| SIRT1 | NAD-dependent deacetylase, which | 4.9 | 0.05 |
| | regulates processes such as apoptosis and | | |
| | muscle differentiation | | |
| C13orf1 | SPla/RYanodine receptor SPRY | 4.9 | 0.03 |
| REPS1 | May coordinate the cellular actions of | 4.9 | 0.02 |
| | activated EGF receptors and Ral-GTPases | | |
| BACH1 | Transcriptional regulator that acts as | 4.8 | 0.02 |
| | repressor or activator. Binds, to NF-E2 | | |
| | binding sites. Coordinates transcription | | |
| | activation/repression by MAFK | | |
| PPP1R13B | Regulator that plays a central role in | 4.8 | 0.03 |
| | regulation of apoptosis via its interaction | | |
| | with p53/TP53. | | |
| PLA1A | Esterase/lipase/thioesterase, active site; | 4.8 | 0.03 |
| ARF1 | GTP-binding protein; allosteric activator | 4.7 | 0.04 |
| | involved in protein trafficking among | | |
| | different compartments. | | |
| CNTN6 | ABC transporter; Fibronectin, type III; | 4.6 | 0.04 |
| | Immunoglobulin-like | | |
| DHRS2 | May inhibit cell replication | 4.6 | 0.05 |
| SYT1 | May have regulatory role in the membrane | 4.5 | 0.02 |
| | interactions during trafficking | | |
| Q8NG51 | Zn-finger, Ran-binding | 4.4 | 0.03 |
| EBF3 | Transcriptional activator which recognizes | 4.4 | 0.05 |
| | variations of the palindromic sequence | | |
| | “ATTCCCNNGGGAATT” | | |
| ITPKB | Inositol-trisphosphate 3-kinase B | 4.3 | 0.03 |
| TCN1 | Vitamin B12-binding protein. Transports | 4.3 | 0.02 |
| | cobalamin into cells | | |
| SULT1C1 | Catalyzes the sulfate conjugation of many | 4.3 | 0.05 |
| | drugs, xenobiotic compounds, hormones, | | |
| | and neurotransmitters. | | |
| LRRN1 | Leucine-rich repeat protein with cysteine- | 4.2 | 0.05 |
| | rich flanking C-terminal region; | | |
| | Fibronectin, type III; Immunoglobulin-like; | | |
| MINK1; MAP4K6 | Serine/threonine kinase that may play a | 4.1 | 0.04 |
| | role in the response to environmental | | |
| | stress. Acts upstream of JUN. | | |
| DBI | Functions as an intracellular carrier of acyl- | 4.1 | 0.04 |
| | CoA esters. May modulate the action of the | | |
| | GABA receptor | | |
| Q14159 | ATP/GTP-binding site motif A (P-loop) | 4.1 | 0.02 |
| ZHX2 | Homeobox; Zn-finger, C2H2 type | 4.1 | 0.05 |
| COLEC12 | Protein C2orf4; C21orf19-like protein | 4.0 | 0.03 |
| SDCCAG33 | Zn-finger, C2H2 type | 4.0 | 0.02 |
| MPHOSPH6 | M-phase phosphoprotein 6 | 4.0 | 0.01 |
| Q86TW0 | Bipartite nuclear localization signal; Zn- | 4.0 | 0.05 |
| | finger, C-x8-C-x5-C-x3-H type | | |
| MFAP1 | Component of the elastin-associated | 4.0 | 0.03 |
| | microfibrils | | |
| KLRB1 | C-type lectin | 4.0 | 0.03 |
| APOL5 | May affect the movement of lipids in cyto- | 4.0 | 0.05 |
| | plasm or allow lipid:organelles binding | | |
| GPR30 | Orphan receptor; possibly for a chemokine | 3.9 | 0.02 |
| HYAL4 | Epidermal growth factor-like domain; | 3.9 | 0.01 |
| | Glycoside hydrolase, family 56; | | |
| CCR7 | Receptor for the MIP3-β chemokine. | 3.9 | 0.02 |
| FOXQ1 | Forkhead box protein Q1; Hepatocyte | 3.9 | 0.04 |
| | nuclear factor 3 forkhead homolog 1; | | |
| VBP1 | Binds specifically to cytosolic chaperonin | 3.8 | 0.01 |
| | (c-CPN) and transfers target proteins to it. | | |
| PSMA1 | Proteasome subunit; a multicatalytic | 3.8 | 0.04 |
| | proteinase complex that cleaves specific | | |
| | peptides | | |
| KLHDC2 | Kelch domain containing protein 2; | 3.8 | 0.04 |
| | Hepatocellular carcinoma-associated | | |
| | antigen 33; Host cell factor homolog LCP | | |
| MRPL37 | Ribosomal protein, mitochondrial, L2. | 3.8 | 0.06 |
| | mitochondrial ribosomal protein L37 | | |
| ARID3A | Binds a VH promoter proximal site | 3.7 | 0.06 |
| | necessary for induced mu-heavy-chain | | |
| | transcription. | | |
| Q8N867 | unknown | 3.7 | 0.05 |
| ASPN | Asporin precursor; Periodontal ligament- | 3.6 | 0.06 |
| | associated protein 1 | | |
| MAP2K4 | Dual specificity kinase that activates the | 3.6 | 0.05 |
| | JUN kinases MAPK8 (JNK1) and MAPK9 | | |
| | (JNK2) as well as MAPK14 (p38) but not | | |
| | MAPK1 (ERK2) or MAPK3 (ERK1) | | |
| ELF1 | Transcription factor that transcriptionally | 3.6 | 0.00 |
| | activates the LYN and mouse BLK | | |
| | promoters | | |
| HIPK2 | Protein kinase acting as a corepressor of | 3.5 | 0.04 |
| | several transcription factors, including | | |
| | SMAD1 and POU4F1/Brn3a and probably | | |
| | NK homeodomain transcription factors. | | |
| | Inhibits cell growth and promotes | | |
| | apoptosis. Wnt/beta-catenin pathway. | | |
| RBM3 | Putative RNA-binding protein 3; | 3.5 | 0.02 |
| TMSB10 | Plays an important role in the organization | 3.4 | 0.05 |
| | of the cytoskeleton. Binds to and sequesters | | |
| | actin monomers (G actin) and therefore | | |
| | inhibits actin polymerization | | |
| BMPR2 | Activin type II receptor; Protein kinase; | 3.4 | 0.06 |
| | TGF-beta receptor/activin receptor, type | | |
| | I/II | | |
| YWHAH | Adapter protein implicated in the | 3.4 | 0.02 |
| | regulation of a large spectrum of both | | |
| | general and specialized signaling pathways. | | |
| PPP1CB | Protein phosphatase (PP1) essential for cell | 3.4 | 0.01 |
| | division, glycogen metabolism, muscle | | |
| | contractility and protein synthesis. | | |
| COX7B | Nuclear-coded polypeptide chain of | 3.4 | 0.01 |
| | cytochrome c oxidase | | |
| C13orf11 | coiled-coil; low complexity; signal peptide; | 3.3 | 0.03 |
| | transmembrane | | |
| Q9H5A9 | 60S Acidic ribosomal protein | 3.3 | 0.02 |
| EIF3S12 | Binds to the 40S ribosome and promotes | 3.3 | 0.01 |
| | the binding of met-tRNAi and mRNA | | |
| KPNB1 | Functions in nuclear protein import, either | 3.3 | 0.06 |
| | in association with an adapter protein, or as | | |
| | an autonomous nuclear transport receptor. | | |
| COL5A1 | Collagen triple helix repeat; | 3.2 | 0.05 |
| GSTK1 | Specific glutathione conjugating activity | 3.2 | 0.05 |
| | with 1-chloro-2,4-dinitrobenzene (CDNB) | | |
| ZNF576 | Zn-finger, C2H2 type | 3.2 | 0.06 |
| Q96T82 | signal peptide; transmembrane | 3.2 | 0.00 |
| C14orf132 | transmembrane | 3.1 | 0.01 |
| BAZ2A | May serve a specific role with ISWI in | 3.1 | 0.05 |
| | maintaining or altering the chromatin | | |
| | structure of the rDNA locus | | |
| Q8N2S5 | Pistil-specific extensin-like protein; | 3.1 | 0.04 |
| | Proline-rich extensin; Proline-rich region | | |
| CBFA2T3 | Proline-rich region; Zn-finger, MYND type | 3.1 | 0.05 |
| MEP1A | Meprin A alpha-subunit precursor; | 3.1 | 0.02 |
| | Endopeptidase-2; | | |
| MARK3 | Involved in the specific phosphorylation of | 3.1 | 0.05 |
| | microtubule-associated proteins | | |
| Q8WUC7 | Bipartite nuclear localization signal | 3.1 | 0.02 |
| Q96BW9 | unknown | 3.0 | 0.04 |
| Q9P1E7 | unknown | 2.9 | 0.05 |
| Q8TF23 | BED finger; Cytochrome c heme-binding | 2.9 | 0.01 |
| | site; KRAB box; Zn-finger, C2H2 subtype | | |
| ETNK2 | Ethanolamine kinase-like protein EKI2 | 2.9 | 0.04 |
| NCK1 | Adapter protein which associates with | 2.9 | 0.03 |
| | tyrosine-phosphorylated growth factor | | |
| | receptors or their cellular substrates | | |
| HPSE | Glycoside hydrolase, family 79, N-terminal | 2.9 | 0.04 |
| RCN1 | May regulate calcium-dependent activities | 2.9 | 0.06 |
| | in the endoplasmic reticulum lumen or | | |
| | post-ER compartment | | |
| Q92519 | Protein kinase | 2.9 | 0.02 |
| PANK3 | Plays a role in the physiological regulation | 2.8 | 0.02 |
| | of the intracellular CoA concentration | | |
| BDNF | Promotes the survival of neuronal | 2.8 | 0.00 |
| | populations | | |
| Q96EC8 | Protein of unknown function DUF649 | 2.8 | 0.06 |
| LY6G6E | Putative Ly-6 superfamily member; | 2.8 | 0.02 |
| | lymphocyte antigen 6 complex, locus G6E | | |
| MTRF1 | Mitochondrial peptide chain release factor | 2.8 | 0.04 |
| | that directs the termination of translation | | |
| Q9NWD5 | Bipartite nuclear localization signal | 2.7 | 0.05 |
| Q8NBE8 | BTB/POZ domain; Kelch repeat | 2.7 | 0.02 |
| CPEB4 | RNA-binding region RNP-1 (RNA | 2.7 | 0.04 |
| | recognition motif) | | |
| Q86X05 | Hly-III related proteins | 2.6 | 0.05 |
| MLCB | Important role in regulation of both smooth | 2.6 | 0.05 |
| | muscle & other cell contractile activity | | |
| SMURF2 | E3 ubiquitin-protein ligase; interacts with | 2.6 | 0.05 |
| | SMAD1, SMAD2 and SMAD7 leading to | | |
| | ubiquitination and degradation. | | |
| Q8WVI0 | low complexity | 2.6 | 0.02 |
| ECGF1 | May have a role in maintaining the | 2.6 | 0.01 |
| | integrity of the blood vessels. Has growth | | |
| | promoting activity on endothelial cells, | | |
| | angiogenic activity in vivo and chemotactic | | |
| | activity on endothelial cells in vitro | | |
| C20orf107 | Similar to neuronal thread protein. | 2.6 | 0.04 |
| GSK3B | Participates in the Wnt signaling pathway. | 2.6 | 0.04 |
| | Phosphorylates JUN, thereby reducing its | | |
| | affinity for DNA | | |
| FIGNL1 | AAA ATPase, central region; ATP/GTP- | 2.6 | 0.00 |
| | binding site motif A (P-loop) | | |
| Q9BRJ9 | Basic helix-loop-helix dimerization domain | 2.6 | 0.04 |
| | bHLH | | |
| DACH2 | Bipartite nuclear localization signal; | 2.5 | 0.02 |
| | Transforming protein Ski | | |
| Q9HCE6 | DH domain | 2.5 | 0.00 |
| ATP2B4 | Mg²⁺-dependent enzyme catalyzes the | 2.5 | 0.03 |
| | hydrolysis of ATP coupled Ca²⁺ efflux | | |
| Q9H631 | Mak10 subunit, NatC N(alpha)-terminal | 2.5 | 0.01 |
| | acetyltransferase | | |
| Q9UF01 | FGF receptor activating protein 1. | 2.5 | 0.05 |
| PTS | Involved in the biosynthesis of | 2.5 | 0.03 |
| | tetrahydrobiopterin, an essential cofactor of | | |
| | aromatic amino acid hydroxylases. | | |
| HBG1 | The epsilon chain is a beta-type chain of | 2.5 | 0.02 |
| | early mammalian embryonic hemoglobin | | |
| ZNF208 | KRAB box; Neutral zinc | 2.4 | 0.05 |
| | metallopeptidases, Zn-finger, C2H2 type | | |
| MLL2 | May be involved in transcriptional | 2.4 | 0.04 |
| | regulation | | |
| NPAS2 | Neuronal PAS domain protein 2; Neuronal | 2.4 | 0.04 |
| | PAS2; Member of PAS protein 4; MOP4 | | |
| ZNF80 | Maybe involved in transcript regulation | 2.4 | 0.02 |
| Q9GZT3 | RNA-binding region RNP-1 (RNA | 2.4 | 0.06 |
| | recognition motif) | | |
| Q8N1Q6 | coiled-coil; low complexity | 2.4 | 0.04 |
| INSIG1 | May play a role in growth & differentiation | 2.4 | 0.03 |
| | of tissues involved in metabolic control. | | |
| DDX39 | ATP/GTP-binding site motif A (P-loop); | 2.3 | 0.05 |
| | DEAD/DEAH box helicase; | | |
| Q8NG48 | WINS1 protein isoform 1. | 2.3 | 0.03 |
| SERPINB8 | Serpin B8; Cytoplasmic antiproteinase 2; | 2.3 | 0.00 |
| | CAP2; CAP-2; Protease inhibitor 8 | | |
| CDC2L1 | Appears to play multiple roles in cell cycle | 2.3 | 0.01 |
| | progression, cytokinesis and apoptosis. | | |
| NCAM2 | May play important roles in selective | 2.3 | 0.02 |
| | fasciculation | | |
| EMD | Emerin | 2.3 | 0.01 |
| PET112L | Formation of correctly charged Gln- | 2.3 | 0.02 |
| | tRNA(Gln) through transamidation. | | |
| MBNL1 | Binds to CUG triplet repeat expansion | 2.3 | 0.05 |
| | dsRNA | | |
| HEY2 | Antifreeze protein, type I; Basic helix-loop- | 2.3 | 0.02 |
| | helix dimerization domain bHLH | | |
| ADRM1 | Promotes cell adhesion | 2.2 | 0.02 |
| MCL1 | Involved in programing of differentiation | 2.2 | 0.02 |
| | and concomitant maintenance of viability | | |
| | but not of proliferation. Isoform 1 inhibits | | |
| | apoptosis while isoform 2 promotes it | | |
| NSMAF | Couples the p55 TNF-receptor (TNFR1) to | 2.2 | 0.03 |
| | neutral sphingomyelinase. | | |
| NDUFB4 | Transfer of electrons from NADH to the | 2.2 | 0.02 |
| | respiratory chain. | | |
| VPREB1 | Associates with the Ig-mu chain to form a | 2.2 | 0.05 |
| | molecular complex that is expressed on the | | |
| | surface of pre-B-cells. Regulates ig gene | | |
| | rearrangements in the early steps of B-cell | | |
| | differentiation | | |
| EIF3S7 | Binds to the 40S ribosome and promotes | 2.2 | 0.05 |
| | the binding of methionyl-tRNAi and | | |
| | mRNA. Associates with the subunit p170 | | |
| | of eIF-3 | | |
| KIAA1404 | Protein KIAA1404 | 2.2 | 0.04 |
| HILS1 | Implicated in chromatin remodeling and/or | 2.2 | 0.04 |
| | transcriptional regulation during | | |
| | spermiogenesis, the process of spermatid | | |
| | maturation into spermatozoa | | |
| HSD3B1 | Crucial role in the biosynthesis of all | 2.2 | 0.02 |
| | classes of hormonal steroids; 3beta-HSD is | | |
| | a bifunctional enzyme, that catalyzes the | | |
| | oxidative conversion of some steroids and | | |
| | the oxidative conversion of ketosteroids. | | |
| Q8IVU3 | Regulator of chromosome condensation, | 2.2 | 0.03 |
| | HECT (Ubiquitin-protein ligase)domain | | |
| Q8WV60 | PPR repeat | 2.2 | 0.01 |
| DAB1 | Adapter molecule functioning in neural | 2.2 | 0.02 |
| | development. May regulate SIAH1 activity | | |
| ICAM3 | ICAM proteins are ligands for the | 2.2 | 0.05 |
| | leukocyte adhesion LFA-1 protein (integrin | | |
| | alpha-L/beta-2). ICAM3 is also a ligand for | | |
| | integrin alpha-D/beta-2 | | |
| WWP2 | E3 ubiquitin-protein ligase which directly | 2.2 | 0.04 |
| | transfers the ubiquitin to targeted substrates | | |
| O14950 | Calcium-binding EF-hand | 2.2 | 0.04 |
| OSGEPL1 | Glycoprotease (M22) metalloprotease | 2.2 | 0.01 |
| RASA2 | Inhibitory regulator of the Ras-cyclic AMP | 2.2 | 0.02 |
| | pathway. Binds inositol tetrakisphosphate | | |
| MAFF | Interacts with the upstream promoter | 2.1 | 0.04 |
| | region of the oxytocin receptor gene. May | | |
| | be a transcriptional enhancer involved in | | |
| | the cellular stress response | | |
| SNAPAP | May have a role in the mechanisms of | 2.1 | 0.05 |
| | SNARE-mediated membrane fusion in | | |
| | non-neuronal cells | | |
| GNA15 | Guanine nucleotide-binding protein (G | 2.1 | 0.05 |
| | proteins) possibly involved as modulator or | | |
| | transducers in transmembrane signaling | | |
| CASR | Senses changes in the extracellular | 2.1 | 0.02 |
| | concentration of calcium ions. The activity | | |
| | of this receptor is mediated by a G-protein | | |
| | that activates a phosphatidylinositol- | | |
| | calcium second messenger system | | |
| POLR2D | DNA-dependent RNA polymerase | 2.1 | 0.05 |
| | Associates with POLR2G | | |
| Q9UPS8 | Ankyrin; RepA/Rep+ protein KID | 2.1 | 0.01 |
| FER1L3 | May play a role in membrane regeneration | 2.1 | 0.02 |
| | and repair | | |
| CCNB1IP1 | E3 ubiquitin ligase. Modulates cyclin B | 2.1 | 0.03 |
| | levels. Overexpression causes delayed | | |
| | entry into mitosis | | |
| GCN5L1 | Biogenesis of lysosome-related organelles | 2.1 | 0.04 |
| | complex-1, subunit 1; | | |
| BTBD2 | BTB/POZ domain containing protein 2 | 2.1 | 0.04 |
| EHHADH | eIF-2 functions in the early steps of protein | 2.1 | 0.00 |
| | synthesis by forming a ternary complex | | |
| | with GTP and initiator tRNA. | | |
| Q96MX1 | down-regulated by Ctnnb1, a. | 2.1 | 0.01 |
| NDUFV1 | Transfer of electrons from NADH to the | 2.1 | 0.01 |
| | respiratory chain. | | |
| Q9NZY8 | unknown | 2.1 | 0.05 |
| CD81 | May play an important role in the | 2.1 | 0.06 |
| | regulation of lymphoma cell growth. | | |
| | Possibly involved in signal transduction. | | |
| | May acts as the viral receptor for HCV | | |
| PAIP2 | PABP-interacting protein 2; polyA-binding | 2.1 | 0.06 |
| | protein-interacting protein 2. | | |
| ATP6V1G2 | Catalytic subunit of the peripheral V1 | 2.1 | 0.04 |
| | complex of vacuolar ATPase. Responsible | | |
| | for acidifying a variety of intracellular | | |
| | compartments in eukaryotic cells | | |
| COL9A2 | Structural component of hyaline cartilage | 2.0 | 0.02 |
| | and vitreous of the eye | | |
| FBXW2 | Substrate-recognition component of the | 2.0 | 0.02 |
| | SCF (SKP1-CUL1-F-box protein)-type E3 | | |
| | ubiquitin ligase complex | | |
| GALK1 | Major enzyme for galactose metabolism | 2.0 | 0.06 |
| O15069 | Nascent polypeptide-associated complex | 2.0 | 0.05 |
| | NAC | | |
| ZNF140 | ATP/GTP-binding site motif A (P-loop); | 2.0 | 0.04 |
| | KRAB box; Zn-finger, C2H2 type | | |
| WFS1 | Wolframin | 2.0 | 0.03 |
| ENSG00000106603 | signal peptide; transmembrane | 2.0 | 0.03 |
| LECT2 | Neutrophil chemotactic activity. Positive | 2.0 | 0.01 |
| | regulator of chondrocyte proliferation | | |
| Q8IYT6 | Bipartite nuclear localization signal | 2.0 | 0.03 |
| RBP5 | Intracellular transport of retinol | 2.0 | 0.04 |
| Q8NAC1 | Protein of unknown function DUF609; | 2.0 | 0.04 |
| | RNA-binding region RNP-1 | | |
| PCLO | May act as a scaffolding protein | 2.0 | 0.06 |
| DBC1 | Deleted in bladder cancer chromosome | 2.0 | 0.01 |
| | region candidate 1. | | |
| STMN2 | May play a role in neuronal differentiation, | 2.0 | 0.02 |
| | and in modulating membrane interaction | | |
| | with the cytoskeleton | | |
| PANK2 | Maybe the master regulator of the CoA | 2.0 | 0.02 |
| | biosynthesis | | |
| Q9NVS3 | IQ calmodulin-binding region | 2.0 | 0.02 |
| ARHGDIA | Regulates the GDP/GTP exchange reaction | 2.0 | 0.03 |
| | of Rho proteins | | |
| Q9H607 | Bipartite nuclear localization signal | 1.9 | 0.03 |
| Q9P233 | Calponin-like actin-binding; Eggshell | 1.9 | 0.04 |
| | protein; Leucine-rich repeat | | |
| DPYS | Dihydropyrimidinase; Hydantoinase; DHP | 1.9 | 0.05 |
| BTBD12 | BTB/POZ domain | 1.9 | 0.03 |
| PELI1 | Scaffold protein involved in the IL-1 | 1.9 | 0.00 |
| | signaling pathway via its interaction with | | |
| | the complex containing IRAK kinases and | | |
| | TRAF6. Required for NFκ-B activation & | | |
| | IL-8 gene expression in response to IL-1 | | |
| TTLL3 | Tubulin tyrosine ligase-like protein 3; | 1.9 | 0.01 |
| | HOTTL | | |
| HDC | Histidine decarboxylase; HDC | 1.9 | 0.04 |
| NOTCH4 | Class II histocompatibility antigen, beta | 1.9 | 0.00 |
| | chain, beta-1 domain; | | |
| TSPAN9 | Tetraspanin-9; Tspan-9; Tetraspan NET-5 | 1.9 | 0.01 |
| Q8TBL3 | NULL | 1.9 | 0.04 |
| Q96NJ4 | signal peptide | 1.9 | 0.05 |
| CD151 | Essential for proper assembly of the | 1.9 | 0.01 |
| | glomerular & tubular basement membranes | | |
| | in kidney | | |
| USP45 | Ubiquitin thiolesterase, family 2; Zn-finger | 1.9 | 0.04 |
| | in ubiquitin thiolesterase | | |
| SLC30A5 | Cation efflux protein | 1.9 | 0.05 |
| SIAT4C | May catalyze the formation of the sugar | 1.9 | 0.00 |
| | sequences found in terminal carbohydrate | | |
| | groups of glycoproteins and glycolipids. | | |
| GBA | Glucosylceramidase precursor; Beta- | 1.9 | 0.03 |
| | glucocerebrosidase; Acid beta-glucosidase; | | |
| AKAP6 | Binds to type II regulatory subunits of | 1.9 | 0.06 |
| | protein kinase A and anchors/targets them | | |
| | to the nuclear membrane or sarcoplasmic | | |
| | reticulum. May act as an adapter for | | |
| | assembling multiprotein complexes | | |
| Q96LI1 | coiled-coil; low complexity | 1.9 | 0.02 |
| Q9Y4C1 | Transcription factor jumonji, jmjC | 1.9 | 0.06 |
| FKBP11 | PPIases accelerate the folding of proteins | 1.8 | 0.00 |
| | during protein synthesis | | |
| C12orf4 | low complexity | 1.8 | 0.01 |
| Q7Z5J8 | Ankyrin; Armadillo repeat; RNA-binding | 1.8 | 0.03 |
| | region RNP-1 (RNA recognition motif) | | |
| Q8NEQ3 | unknown | 1.8 | 0.02 |
| MAP2K6 | Catalyzes the concomitant phosphorylation | 1.8 | 0.01 |
| | of a threonine and a tyrosine residue in | | |
| | MAP kinase p38 exclusively | | |
| CRIM1 | Antistasin; Eukaryotic thiol (cysteine) | 1.8 | 0.04 |
| | protease; | | |
| MUC1 | Mucin, May play a role in adhesive | 1.8 | 0.01 |
| | functions and in cell-cell interactions, | | |
| | metastasis and signaling. Possible protective | | |
| | layer on epithelial surfaces. Direct or | | |
| | indirect interaction with actin cytoskeleton | | |
| F13A1 | Factor XIII stabilizes fibrin clots. Also | 1.8 | 0.02 |
| | cross-link alpha-2-plasmin inhibitor, or | | |
| | fibronectin, to the alpha chains of fibrin | | |
| MRPS28 | Mitochondrial 28S ribosomal protein S28; | 1.8 | 0.05 |
| | S28mt; MRP-S28; MRP-S35 | | |
| FYB | Acts as an adapter protein of the FYN and | 1.8 | 0.03 |
| | SH2-domain-containing leukocyte protein- | | |
| | 76 (SLP76) signaling cascades in T cells. | | |
| | Modulates the expression of interleukin-2 | | |
| TFR2 | Mediates cellular uptake of transferrin- | 1.8 | 0.01 |
| | bound iron in a non-iron dependent | | |
| | manner. | | |
| Q96PY3 | Leucine-rich repeat | 1.8 | 0.03 |
| TRAPPC3 | May play a role in vesicular transport from | 1.8 | 0.04 |
| | endoplasmic reticulum to Golgi | | |
| Q9NUQ9 | NULL | 1.8 | 0.04 |
| O60844 | Jacalin-related lectin | 1.8 | 0.02 |
| PTPRF | Possible cell adhesion receptor; has intrinsic | 1.8 | 0.00 |
| | protein tyrosine phosphatase activity | | |
| SOX6 | Binds specifically to the DNA sequence | 1.8 | 0.02 |
| | “AACAAT-3” | | |
| RAGE | Able to phosphorylate several exogenous | 1.8 | 0.03 |
| | substrates & undergo autophosphorylation | | |
| PLAUR | Receptor for urokinase plasminogen | 1.8 | 0.01 |
| | activator. Plays a role in localizing and | | |
| | promoting plasmin formation. | | |
| ENTPD5 | Likely to promote reglycosylation reactions | 1.8 | 0.02 |
| | involved in glycoprotein folding & quality | | |
| | control in the endoplasmic reticulum. | | |
| UNC5B | ATP/GTP-binding site motif A (P-loop); | 1.8 | 0.04 |
| | Death domain; Immunoglobulin-like | | |
| CHCHD3 | Protein of unknown function DUF737 | 1.8 | 0.03 |
| LMNA | Lamins are components of the nuclear | 1.8 | 0.05 |
| | lamina, a fibrous layer on the | | |
| | nucleoplasmic side of the inner nuclear | | |
| | membrane that is thought to provide a | | |
| | framework for the nuclear envelope | | |
| ZNF254 | May function as a transcription factor | 1.8 | 0.03 |
| POLR1B | RNA polymerase beta subunit | 1.8 | 0.02 |
| RUFY2 | Cytochrome c heme-binding site; | 1.8 | 0.06 |
| CDC34 | Catalyzes the covalent attachment of | 1.8 | 0.04 |
| | ubiquitin to other proteins | | |
| DEF6 | Calcium-binding EF-hand; Pleckstrin-like; | 1.8 | 0.00 |
| | Tropomyosin | | |
| PKHD1L1 | ATP/GTP-binding site motif A (P-loop); | 1.8 | 0.05 |
| | Cell surface receptor IPT/TIG | | |
| FGL1 | Has hepatocyte mitogenic activity | 1.8 | 0.01 |
| TRPC7 | Thought to form a receptor-activated non- | 1.8 | 0.05 |
| | selective calcium permeant cation channel. | | |
| GLUL | Glutamine synthetase; | 1.8 | 0.00 |
| IGHG3 | Ig alpha is the major immunoglobulin class | 1.8 | 0.01 |
| | in body secretions. It may serve both to | | |
| | defend against local infection and to | | |
| | prevent access of foreign antigens to the | | |
| | general immunologic system | | |
| DSPG3 | May have a role in bone formation and also | 1.8 | 0.03 |
| | in establishing the ordered structure of | | |
| | cartilage through matrix organization | | |
| C10orf82 | unknown | 1.8 | 0.05 |
| ENSG00000144872 | Ribosomal protein L39e | 1.7 | 0.01 |
| FRMD1 | Band 4.1 domain | 1.7 | 0.01 |
| Q96J64 | Somatomedin B; Thrombospondin, type I | 1.7 | 0.05 |
| PCBD | Involved in tetrahydrobiopterin | 1.7 | 0.01 |
| | biosynthesis. | | |
| INPP4A | Inositol polyphosphate-4-phosphatase I | 1.7 | 0.04 |
| TRIM39 | Tripartite motif protein 39; RING finger | 1.7 | 0.04 |
| | protein 23; Testis-abundant finger protein | | |
| AKAP11 | Binds to type II regulatory subunits of | 1.7 | 0.03 |
| | protein kinase A and anchors/targets them | | |
| ZNF292 | May function as a transcription factor | 1.7 | 0.04 |
| APG10L | Autophagocytosis associated protein, C- | 1.7 | 0.00 |
| | terminal | | |
| ZNF177 | May be involved in transcriptional | 1.7 | 0.05 |
| | regulation | | |
| TSHR | Receptor for thyrothropin. Plays a central | 1.7 | 0.01 |
| | role in controlling thyroid cell metabolism. | | |
| EAF1 | Proline-rich extensin; Proline-rich region | 1.7 | 0.02 |
| Q8N2H5 | Adrenodoxin reductase; Regulator of | 1.7 | 0.02 |
| | chromosome condensation | | |
| ETV4 | Ets-domain; PEA3-type ETS-domain | 1.7 | 0.01 |
| | transcription factor, N-terminal | | |
| ACE2 | Neutral zinc metallopeptidase | 1.7 | 0.05 |
| NTRK3 | Tyrosine-protein kinase receptor for | 1.7 | 0.00 |
| | neurotrophin-3 (NT-3). Known substrates | | |
| | for trk receptors are SHC1, PI-3 kinase, | | |
| | and PLCG1. | | |
| FADS1 | Cytochrome b5; Fatty acid desaturase | 1.7 | 0.01 |
| | family | | |
| Q9H679 | low complexity; signal peptide; | 1.7 | 0.01 |
| | transmembrane | | |
| RBMS3 | Paraneoplastic encephalomyelitis antigen; | 1.7 | 0.01 |
| | RNA-binding region RNP-1 | | |
| C20orf173 | Hypothetical protein C20orf173 | 1.7 | 0.06 |
| Q9BY88 | Calcium-binding EF-hand | 1.7 | 0.01 |
| USP6NL | RabGAP/TBC domain | 1.7 | 0.04 |
| NDUFB7 | Transfer of electrons from NADH to the | 1.7 | 0.03 |
| | respiratory chain. | | |
| FRZB | Soluble frizzled-related proteins (sFRPS) | 1.7 | 0.01 |
| | function as modulators of Wnt signaling. | | |
| | They have a role in regulating cell growth | | |
| | and differentiation in specific cell types. | | |
| Q9NTD9 | unknown | 1.7 | 0.04 |
| ZFP36 | Probable regulatory protein with a novel | 1.7 | 0.05 |
| | zinc finger structure involved in regulating | | |
| | the response to growth factors. | | |
| PPM1D | Required for the relief of p53-dependent | 1.7 | 0.03 |
| | checkpoint mediated cell cycle arrest. | | |
| Q8NBM8 | NULL | 1.7 | 0.01 |
| STAT1 | Signal transducer and activator of | 1.7 | 0.04 |
| | transcription that mediates signaling by | | |
| | interferons (IFNs). | | |
| DSC1 | Component of intercellular desmosome | 1.7 | 0.01 |
| | junctions. Involved in the interaction of | | |
| | plaque proteins and intermediate filaments | | |
| | mediating cell-cell adhesion. | | |
| GMEB2 | Trans-acting factor that binds to | 1.7 | 0.05 |
| | glucocorticoid modulatory elements. Binds | | |
| | also to the transferrin receptor promoter. | | |
| PPIC | Catalyzes the cis-trans isomerization of | 1.7 | 0.03 |
| | proline imidic peptide bonds in oligo- | | |
| | peptides to accelerate protein folding | | |
| ARRDC2 | Arrestin | 1.7 | 0.01 |
| ADPRHL1 | ADP-ribosylglycohydrolase | 1.7 | 0.01 |
| HES7 | Basic helix-loop-helix dimerization domain | 1.7 | 0.00 |
| | bHLH; Proline-rich extensin; | | |
| Wdr68; Han11 | WD-repeat protein 68; WD-repeat protein | 1.7 | 0.02 |
| | An11 homolog | | |
| PSMB7 | Proteasome subunit | 1.7 | 0.01 |
| SLC25A20 | Transport of acylcarnitines of different | 1.6 | 0.05 |
| | length across the mitochondrial inner | | |
| | membrane for their oxidation | | |
| CACNA2D2 | Cache domain; von Willebrand factor, type A | 1.6 | 0.05 |
| EDG2 | Receptor for lysophosphatidic acid (LPA), | 1.6 | 0.00 |
| | a mediator of diverse cellular activities. | | |
| | Coupled to heteromeric G proteins | | |
| TFEC | Basic helix-loop-helix dimerization domain | 1.6 | 0.04 |
| | bHLH | | |
| ETF1 | Directs the termination of nascent peptide | 1.6 | 0.01 |
| | synthesis (translation) | | |
| NP | Purine nucleoside phosphorylase; Inosine | 1.6 | 0.00 |
| | phosphorylase; PNP | | |
| SLC11A1 | Divalent transition metal (iron and | 1.6 | 0.04 |
| | manganese) transporter involved in iron | | |
| | metabolism and host resistance to certain | | |
| | pathogens. Controls natural resistance to | | |
| | infection with intracellular parasites. | | |
| Q8NDJ4 | TBP-interacting 120 | 1.6 | 0.02 |
| TRPV1 | Ankyrin; Ion transport protein | 1.6 | 0.05 |
| CORO1A | May be a crucial component of the | 1.6 | 0.03 |
| | cytoskeleton of highly motile cells | | |
| CRHBP | Binds CRF and inactivates it. May prevent | 1.6 | 0.04 |
| | inappropriate pituitary-adrenal stimulation | | |
| | in pregnancy | | |
| RPO2TC1; PC4 | General coactivator that functions | 1.6 | 0.00 |
| | cooperatively with TAFs and mediates | | |
| | functional interactions between upstream | | |
| | activators and the general transcriptional | | |
| | machinery. Binds single-stranded DNA | | |
| FMNL2 | Actin-binding FH2; Proline-rich extensin; | 1.6 | 0.05 |
| | Wilm's tumour protein | | |
| FZD10 | Receptor for Wnt proteins. May be | 1.6 | 0.05 |
| | involved in transduction and intercellular | | |
| | transmission of polarity information during | | |
| | tissue morphogenesis and/or in | | |
| | differentiated tissues | | |
| MAN1C1 | Involved in the maturation of Asn-linked | 1.6 | 0.02 |
| | oligosaccharides. | | |
| PLAC8 | Placenta-specific gene 8 protein; C15 | 1.6 | 0.04 |
| | protein | | |
| C6orf211 | Protein of unknown function DUF89 | 1.6 | 0.01 |
| PTPN18 | Differentially dephosphorylate auto- | 1.6 | 0.00 |
| | phosphorylated tyrosine kinases which are | | |
| | known to be overexpressed in tumor tissues | | |
| LRP11 | Low density lipoprotein-receptor, class A | 1.6 | 0.05 |
| ITPA | Hydrolyzes ITP and dITP to their | 1.6 | 0.06 |
| | respective monophosphate derivatives. | | |
| | May be the major enzyme responsible for | | |
| | regulating ITP concentration in cells | | |
| COG3 | Involved in ER-Golgi transport | 1.6 | 0.00 |
| CFL2 | Controls reversibly actin polymerization | 1.6 | 0.02 |
| | and depolymerization in a pH-sensitive | | |
| | manner. Major component of intranuclear | | |
| | and cytoplasmic actin rods | | |
| Q8N5X0 | low complexity; signal peptide; | 1.6 | 0.01 |
| | transmembrane | | |
| SIAT8B | May transfer sialic acid to N-linked | 1.6 | 0.03 |
| | oligosaccharides of glycoproteins | | |
| VARS2 | Valyl-tRNA synthetase | 1.6 | 0.01 |
| RAB13 | Could participate in polarized transport, in | 1.6 | 0.05 |
| | assembly and/or activity of tight junctions | | |
| TGIF | Active transcriptional corepressor of | 1.6 | 0.02 |
| | SMAD2. May participate in the | | |
| | transmission of nuclear signals during | | |
| | development and in the adult | | |
| KIF13A | Plus end-directed microtubule-dependent | 1.6 | 0.04 |
| | motor protein involved in mannnose-6- | | |
| | phosphate receptor transport to the plasma | | |
| | membrane | | |
| RGS17 | Inhibits signal transduction by increasing | 1.6 | 0.03 |
| | the GTPase activity of G protein alpha | | |
| | subunits thereby driving them into their | | |
| | inactive GDP-bound form | | |
| ENSG00000180649 | RNA-binding region RNP-1 (RNA | 1.6 | 0.05 |
| | recognition motif) | | |
| Q9UFK2 | low complexity; signal peptide; | 1.6 | 0.05 |
| | transmembrane | | |
| Q9Y6U7 | Proline-rich region; Zn-finger, RING | 1.6 | 0.05 |
| NOL4 | Nucleolar protein 4 | 1.6 | 0.05 |
| PTBP1 | Plays a role in pre-mRNA splicing. Binds | 1.6 | 0.01 |
| | to the polypyrimidine tract of introns. | | |
| Q9NW32 | low complexity | 1.6 | 0.01 |
| RTTN | Rotatin. | 1.6 | 0.06 |
| CDCA4 | May be involved in molecular regulation of | 1.6 | 0.01 |
| | hematopoietic stem cells and progenitor | | |
| | cell lineage commitment and differentiation | | |
| TBC1D14 | RabGAP/TBC domain | 1.6 | 0.01 |
| Q9H693 | low complexity | 1.6 | 0.00 |
| RGS11 | Inhibits signal transduction by increasing | 1.6 | 0.05 |
| | the GTPase activity of G protein alpha | | |
| | subunits thereby driving them into their | | |
| | inactive GDP-bound form | | |
| GIMAP2; | GTPase, IMAP family member 2; | 1.6 | 0.01 |
| IMAP2 | Immunity-associated protein 2; hIMAP2 | | |
| SSBP3 | May be involved in transcription regulation | 1.6 | 0.06 |
| | of the alpha 2(I) collagen gene where it | | |
| | binds to the single-stranded polypyrimidine | | |
| | sequences in the promoter region | | |
| EIF4EL3 | Recognizes and binds the 7- | 1.6 | 0.01 |
| | methylguanosine-containing mRNA cap | | |
| | during an early step in the initiation of | | |
| | protein synthesis and facilitates ribosome | | |
| | binding by inducing the unwinding of the | | |
| | mRNAs secondary structures | | |
| MNDA | May act as a transcriptional | 1.6 | 0.04 |
| | activator/repressor in the myeloid lineage. | | |
| | Plays a role in the granulocyte/monocyte | | |
| | cell-specific response to interferon. | | |
| | Stimulates the DNA binding of the | | |
| | transcriptional repressor protein YY1 | | |
| O95053 | low complexity | 1.6 | 0.01 |
| GNA13 | Guanine nucleotide-binding proteins (G | 1.6 | 0.02 |
| | proteins) are involved as modulators or | | |
| | transducers in various transmembrane | | |
| | signaling systems | | |
| C13orf17 | Protein of unknown function DUF298 | 1.6 | 0.02 |
| EXOSC4 | Component of exosome:exoribonuclease | 1.6 | 0.01 |
| | complex. Required for the processing of | | |
| | the 7S pre-RNA to the mature 5.8S rRNA. | | |
| | Has an exonuclease activity | | |
| O76052 | low complexity | 1.6 | 0.03 |
| F11R | Seems to plays a role in epithelial tight | 1.5 | 0.05 |
| | junction formation. Plays a role in | | |
| | regulating monocyte transmigration | | |
| | involved in integrity of epithelial barrier. | | |
| | Involved in platelet activation | | |
| SLC35B3 | CGI-19 protein; solute carrier family 35, | 1.5 | 0.04 |
| | member B3 | | |
| Q8WVP6 | low complexity | 1.5 | 0.05 |
| Q9NYY8 | NULL | 1.5 | 0.01 |
| SLC9A7 | Na+/H+ exchanger, isoform 6 (NHE6); | 1.5 | 0.06 |
| | Sodium/hydrogen exchanger; | | |
| FUT1 | Creates a soluble precursor oligosaccharide | 1.5 | 0.02 |
| | FuC-α(1,2)Galβ-called the H antigen | | |
| DOT1L | Histone methyltransferase. Methylates Lys- | 1.5 | 0.04 |
| | 79 of histone H3. Nucleosomes are | | |
| | preferred as substrates, cf. free histones | | |
| PPP2R2C | The B regulatory subunit might modulate | 1.5 | 0.02 |
| | substrate selectivity and catalytic activity, | | |
| KIAA2010 | EVH1; Protein of unknown function | 1.5 | 0.05 |
| | DUF625 | | |
| Q9BU62 | Similar to DNA segment, Chr 17, human | 1.5 | 0.02 |
| | D6S51E. | | |
| PPP2CA | PP2A can modulate the activity of | 1.5 | 0.05 |
| | phosphorylase B kinase casein kinase 2, | | |
| | mitogen-stimulated S6 kinase, and MAP-2 | | |
| | kinase. | | |
| SLC22A11 | General substrate transporter | 1.5 | 0.04 |
| MAN2B1 | Necessary for the catabolism of N-linked | 1.5 | 0.02 |
| | carbohydrates released during glycoprotein | | |
| | turnover. Cleaves all known types of alpha- | | |
| | mannosidic linkages | | |
| O94940 | SAM (and some other nucleotide) binding | 1.5 | 0.04 |
| | motif | | |
| APOBEC3G | APOBEC-like, C-terminal; Cytidine/ | 1.5 | 0.03 |
| | deoxycytidylate deaminase, | | |
| Q14843 | Calcium-binding EF-hand | 1.5 | 0.04 |
| Q9P1G3 | Protein of unknown function DUF185 | 1.5 | 0.06 |
| KCNN3 | Voltage-independent potassium channel | 1.5 | 0.01 |
| | activated by intracellular calcium. | | |
| | Activation is followed by membrane | | |
| | hyperpolarization. | | |
| HSD17B12 | Glucose/ribitol dehydrogenase; Short-chain | 1.5 | 0.02 |
| | dehydrogenase/reductase SDR | | |
| ZNF83 | May be involved in transcriptional | 1.5 | 0.02 |
| | regulation | | |
| SIAT8E | May be involved in the synthesis of | 1.5 | 0.02 |
| | gangliosides GD1c, GT1a, GQ1b and GT3 | | |
| PHLDB2 | Pleckstrin-like | 1.5 | 0.01 |
| Q9H2V5 | Bipartite nuclear localization signal | 1.5 | 0.04 |
| AKAP13 | Stimulates exchange activity on Rho | 1.5 | 0.01 |
| | proteins in vitro | | |
| CRHR1 | Receptor for corticotropin releasing factor. | 1.5 | 0.01 |
| Q9BVM2 | DPCD protein. | 1.5 | 0.01 |
| HOXD3 | Sequence-specific transcription factor, part | 1.5 | 0.00 |
| | of a developmental regulatory system | | |
| HDAC9 | Responsible for the deacetylation of lysine | 1.5 | 0.01 |
| | residues on the N-terminal part of the core | | |
| | histones (H2A, H2B, H3 and H4). | | |
| HV2G_HUMAN | Ig heavy chain V-II region NEWM | 1.5 | 0.04 |
| ZNF514 | KRAB box; Zn-finger, C2H2 subtype; | 1.5 | 0.00 |
| CPS1 | Involved in the urea cycle in removing | 1.5 | 0.03 |
| | excess ammonia from the cell | | |
| TTC12 | Tetratricopeptide repeat protein 12 TPR | 1.5 | 0.01 |
| PLXNA3 | Putative receptor involved in the | 1.5 | 0.00 |
| | development of neural & epithelial tissues | | |
| CFLAR | Apoptosis regulator protein which may | 1.5 | 0.01 |
| | function as a crucial link between cell | | |
| | survival and cell death pathways in | | |
| | mammalian cells. Inhibitor of TNFRSF6 | | |
| | mediated apoptosis. | | |
| TCBA1 | T-cell lymphoma breakpoint-associated | 1.5 | 0.01 |
| | target 1. | | |
| RCE1 | Proteolytically removes the C-terminal | 1.5 | 0.00 |
| | three residues of farnesylated and | | |
| | geranylated proteins. Processes K-Ras, N- | | |
| | Ras, H-Ras, RAP1B and G-gamma-1 | | |
| PLA2G5 | PA2 catalyzes the calcium-dependent | −1.5 | 0.02 |
| | hydrolysis of the 2-acyl groups in 3-sn- | | |
| | phosphoglycerides. May be involved in the | | |
| | production of lung surfactant | | |
| STX16 | SNARE involved in a vesicular transport | −1.5 | 0.00 |
| | step within the Golgi stack | | |
| TGM5 | Catalyzes the cross-linking of proteins and | −1.5 | 0.00 |
| | the conjugation of polyamines to proteins. | | |
| Q8N7V2 | low complexity | −1.5 | 0.00 |
| SLC35F3 | Solute carrier family 35, member F3. | −1.5 | 0.04 |
| Q96LR7 | unknown | −1.5 | 0.01 |
| HRMT1L3 | Probably methylates the guanidino | −1.5 | 0.04 |
| | nitrogens of Arg residues in some proteins | | |
| Q9NUJ7 | Phosphatidylinositol-specific | −1.5 | 0.03 |
| | phospholipase C, X domain | | |
| SLC1A6 | Transports L-glutamate and also L- and D- | −1.5 | 0.05 |
| | aspartate, in symport with Na⁺ | | |
| KIAA1024 | UPF0258 protein KIAA1024 | −1.5 | 0.04 |
| MAML3 | CAG repeat containing (glia-derived nexin | −1.5 | 0.00 |
| | I alpha); | | |
| FMO5 | In contrast with other forms of FMO it does | −1.5 | 0.01 |
| | not seem to be a drug-metabolizing enzyme | | |
| Q9BTD3 | hole protein. | −1.5 | 0.00 |
| COL5A2 | Collagen type II specific for cartilaginous | −1.5 | 0.02 |
| | tissues. It is a minor connective tissue | | |
| | component of nearly ubiquitous | | |
| | distribution. | | |
| CD48 | Ligand for CD2. Might facilitate inter- | −1.5 | 0.03 |
| | action between activated lymphocytes. | | |
| | Probably involved in regulating T-cell | | |
| | activation | | |
| NDUFAF1 | Chaperone protein involved in assembly of | −1.5 | 0.05 |
| | the mitochondrial NADH:ubiquinone | | |
| | oxidoreductase complex | | |
| Q9NWV6 | Protein kinase | −1.5 | 0.02 |
| Q9C0D5 | ATP/GTP-binding site motif A (P-loop); | −1.5 | 0.01 |
| | Ankyrin; TPR repeat | | |
| TRIM11 | Tripartite motif protein 17; Testis RING | −1.5 | 0.05 |
| | finger protein; RING finger protein 16 | | |
| ENSG00000187700 | low complexity | −1.5 | 0.04 |
| Q9H7M9 | Immunoglobulin-like | −1.5 | 0.04 |
| IL12A | Cytokine that can act as a growth factor for | −1.5 | 0.03 |
| | activated T and NK cells, enhance the lytic | | |
| | activity of NK/lymphokine-activated Killer | | |
| | cells, and stimulate the production of IFN- | | |
| | gamma by resting PBMC | | |
| Q8IZC1 | Protein of unknown function DUF634 | −1.6 | 0.03 |
| GRSF1 | Binds RNAs containing the 14 base G-rich | −1.6 | 0.03 |
| | element | | |
| NBR2 | Protein NBR2; Next to BRCA1 gene 2 | −1.6 | 0.02 |
| | protein | | |
| Q8TF23 | BED finger; Cytochrome c heme-binding | −1.6 | 0.01 |
| | site; KRAB box; Zn-finger, C2H2 subtype; | | |
| ZNF78L1 | May function as a transcription factor | −1.6 | 0.01 |
| ENSG00000184319 | Bipartite nuclear localization signal; | −1.6 | 0.01 |
| | Ribosomal L23 protein; Ribosomal protein | | |
| | L23, N-terminal domain | | |
| ARID1A | Involved in transcriptional activation and | −1.6 | 0.01 |
| | repression of select genes by chromatin | | |
| | remodeling | | |
| PRKWNK4 | Regulates the activity of the thiazide- | −1.6 | 0.02 |
| | sensitive Na—Cl cotransporter, SLC12A3, | | |
| | by phosphorylation | | |
| EXTL2 | Glycosyltransferase required for the | −1.6 | 0.06 |
| | biosynthesis of heparan-sulfate | | |
| ASPH | Aspartyl/Asparaginyl beta-hydroxylase | −1.6 | 0.02 |
| Q9NV64 | low complexity; transmembrane | −1.6 | 0.01 |
| Q96MY4 | low complexity | −1.6 | 0.02 |
| ADAM30 | May be involved in spermatogenesis and | −1.6 | 0.03 |
| | fertilization | | |
| EFHD1 | EF-hand domain-containing protein 1; | −1.6 | 0.02 |
| | Swiprosin-2 | | |
| SGTB | Small glutamine-rich tetratricopeptide | −1.6 | 0.03 |
| | repeat-containing protein B; | | |
| Q9H0J1 | low complexity; transmembrane | −1.6 | 0.03 |
| ENSG00000185305 | ATP/GTP-binding site motif A (P-loop) | −1.6 | 0.03 |
| A2BP1 | Ataxin-2-binding protein 1 | −1.6 | 0.06 |
| Q86XE5 | Dihydrodipicolinate synthetase | −1.6 | 0.03 |
| FOXK2 | May be involved in both positive and | −1.6 | 0.03 |
| | negative regulation of important viral and | | |
| | cellular promoter elements | | |
| NFIA | Recognizes and binds the palindromic | −1.6 | 0.04 |
| | sequence “TTGGCNNNNNGCCAA”; | | |
| | present in viral and cellular promoters. | | |
| ITGA9 | Integrin alpha-9/beta-1 is a receptor for | −1.6 | 0.03 |
| | VCAM1, cytotactin and osteopontin. | | |
| ZCCHC7 | Zn-finger, CCHC type | −1.6 | 0.00 |
| DOCK1 | Involved in cytoskeletal rearrangements | −1.6 | 0.00 |
| | required for phagocytosis of apoptotic cells | | |
| | and cell motility. Functions as a guanine | | |
| | nucleotide exchange factor (GEF), which | | |
| | activates Rac Rho small GTPases. | | |
| ENSG00000186409 | coiled-coil; low complexity | −1.6 | 0.01 |
| Q8IXL9 | IQ calmodulin-binding region | −1.6 | 0.03 |
| EIF2AK3 | Phosphorylates the alpha subunit of | −1.6 | 0.03 |
| | eukaryotic translation-initiation factor 2 | | |
| | (EIF2) | | |
| Q8NDC9 | Initiation factor 2B | −1.6 | 0.04 |
| SH3BGR | SH3 domain-binding glutamic acid-rich | −1.6 | 0.03 |
| | protein; | | |
| CDY1 | Testis-specific chromodomain protein Y 1 | −1.6 | 0.03 |
| Q8TAZ0 | Ubiquitin system component Cue | −1.6 | 0.01 |
| REV1L | UMUC-like DNA-repair protein | −1.7 | 0.02 |
| Q96IR7 | unknown | −1.7 | 0.06 |
| SMCR8 | Smith-Magenis syndrome chromosome | −1.7 | 0.02 |
| | region, candidate 8. | | |
| ABCA6 | ABC transporter; ATP/GTP-binding site | −1.7 | 0.02 |
| | motif A (P-loop) | | |
| NRIP1 | Modulates transcriptional activation by | −1.7 | 0.01 |
| | steroid receptors such as NR3C1, NR3C2 | | |
| | and ESR1. Also modulates transcriptional | | |
| | repression by nuclear hormone receptors | | |
| O95792 | Zn-finger, A20-like; Zn-finger, AN1-like | −1.7 | 0.03 |
| ITGA5 | Integrin alpha-5/beta-1 is a receptor for | −1.7 | 0.05 |
| | fibronectin & fibrinogen; recognizes RGD | | |
| RPL10A | 60S ribosomal protein L10a | −1.7 | 0.05 |
| PTPRB | Receptor-type tyrosine-protein phosphatase | −1.7 | 0.02 |
| | beta precursor; Protein-tyrosine | | |
| | phosphatase beta; R-PTP-beta | | |
| ADCY9 | May play a fundamental role in situations | −1.7 | 0.02 |
| | where interplay between intracellular Ca²⁺ | | |
| | and cAMP determines the cellular function. | | |
| AKAP13 | Stimulates exchange activity on Rho | −1.7 | 0.05 |
| | proteins in vitro, but not on CDC42, Ras or | | |
| | Rac. May bind calcium ions | | |
| O94948 | RUN domain | −1.7 | 0.05 |
| STK39 | May act as a mediator of stress-activated | −1.7 | 0.01 |
| | signals | | |
| TFB1M | Immunoglobulin/major histocompatibility | −1.7 | 0.05 |
| | complex; Ribosomal RNA adenine | | |
| | dimethylase | | |
| NJMU_HUMAN | May have a role in spermatogenesis | −1.7 | 0.05 |
| Q8N4S7 | Hly-III related proteins | −1.7 | 0.03 |
| ENSG00000166737 | low complexity; transmembrane | −1.7 | 0.01 |
| Q8WWN8 | Human Rev interacting-like protein (hRIP); | −1.7 | 0.02 |
| | Pleckstrin-like; RA domain; RhoGAP | | |
| | domain; Sterile alpha motif SAM | | |
| Q9Y2M0 | Bipartite nuclear localization signal | −1.7 | 0.01 |
| Q9BXY2 | Protein kinase; Tyrosine protein kinase | −1.8 | 0.03 |
| TRPM4 | Ion transport protein | −1.8 | 0.04 |
| NALP1 | Able to form cytoplasmic structures termed | −1.8 | 0.04 |
| | death effector filaments. Enhances APAF1 | | |
| | and cytochrome c-dependent activation of | | |
| | pro-caspase-9 and consecutive apoptosis. | | |
| NAALAD2 | Has N-acetylated-alpha-linked-acidic | −1.8 | 0.01 |
| | dipeptidase (NAALADase) activity. | | |
| GPR55 | Rhodopsin-like GPCR superfamily | −1.8 | 0.00 |
| DKK1 | Inhibitor of Wnt signaling pathway | −1.8 | 0.01 |
| GAS1 | Specific growth arrest protein involved in | −1.8 | 0.01 |
| | growth suppression. | | |
| PCDH9 | Potential calcium-dependent cell-adhesion | −1.9 | 0.01 |
| | protein | | |
| APBA3 | May modulate processing of the β-amyloid | −1.9 | 0.05 |
| | precursor protein (APP) and hence | | |
| | formation of beta-APP | | |
| WBSCR19 | Bipartite nuclear localization signal; | −1.9 | 0.01 |
| | Connexins | | |
| PPFIA3 | Sterile alpha motif SAM | −1.9 | 0.03 |
| UPK2 | Component of the asymmetric unit | −1.9 | 0.01 |
| | membrane (AUM); a highly specialized | | |
| | biomembrane elaborated by terminally | | |
| | differentiated urothelial cells. | | |
| TFCP2L3 | CP2 transcription factor | −1.9 | 0.01 |
| Q9H8Y5 | Ankyrin; Prenyl group binding site (CAAX | −1.9 | 0.03 |
| | box); Zn-finger, C2H2 type | | |
| ADHFE1 | Iron-containing alcohol dehydrogenase | −1.9 | 0.01 |
| O14804 | Aminotransferase, class-II; Beta-ketoacyl | −1.9 | 0.02 |
| | synthase; G-protein coupled receptors | | |
| | family 2 (secretin-like); | | |
| ZNF208 | May function as a transcription factor | −2.0 | 0.00 |
| Q9H5D6 | low complexity | −2.0 | 0.01 |
| TMF1 | This protein binds the HIV-1 TATA | −2.1 | 0.01 |
| | element and inhibits transcriptional | | |
| | activation by the TATA-binding protein | | |
| SGCG | Component of the sarcoglycan complex, | −2.1 | 0.01 |
| | which forms a link between the F-actin | | |
| | cytoskeleton and the extracellular matrix | | |
| KLHL21; | Kelch-like protein 21 | −2.2 | 0.06 |
| ADRA2A | Alpha-2 adrenergic receptors mediate the | −2.2 | 0.04 |
| | catecholamine-induced inhibition of | | |
| | adenylate cyclase through the action of G | | |
| | proteins. | | |
| ENSG00000140876 | NUDIX hydrolase | −2.2 | 0.06 |
| SHANK1 | Seems to be an adapter protein that may | −2.2 | 0.04 |
| | play a role in the structural and functional | | |
| | organization of the dendritic spine and | | |
| | synaptic junction | | |
| Q8IY68 | low complexity | −2.3 | 0.01 |
| NR4A1 | Orphan nuclear receptor | −4.2 | 0.05 |

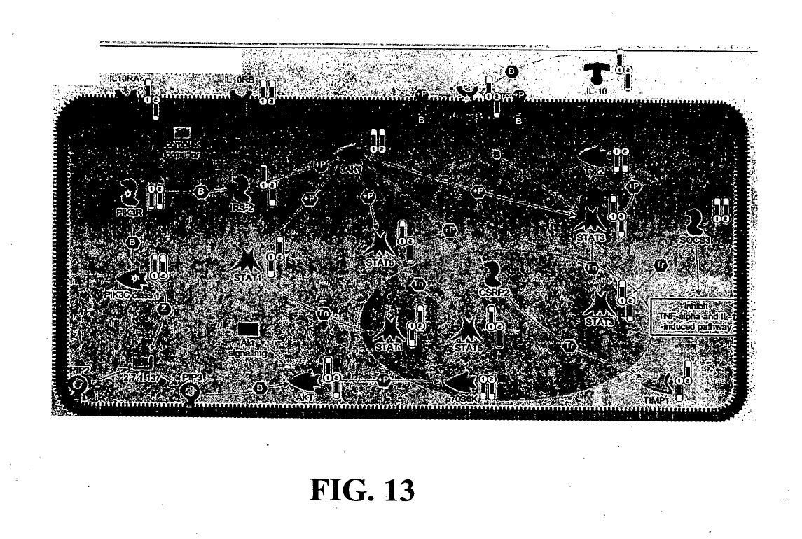

Bioinformatic analysis of the microarray data by supervised clustering of the differentially expressed genes induced by the peptide, followed by mapping of the responses to signal transduction pathways indicated that the synthetic peptide can potentially induce signaling, and activate JAK (Janus Kinases) and the STAT (signal transducers and activators of transcription) family of transcription factors. It had been previously described that the human host defense peptide LL-37 activates MAP kinases (FIG. 4) but the JAK-STAT pathway had not been implicated. These microarray data showed evidence that SEQ ID NO: 7 has the potential to differentially induce transcriptional responses of the genes upstream and downstream of the JAK and STAT family of transcription factors (FIG. 13), as well as activate PI3-Kinase an important modulator of extracellular signals. The peptide (SEQ ID NO:7) also significantly up-regulated the expression regulators of NFκB pathway e.g. TIRAP and NFκB2. This observation reveals a mechanism for the intervention of host defense peptides in immune responses.

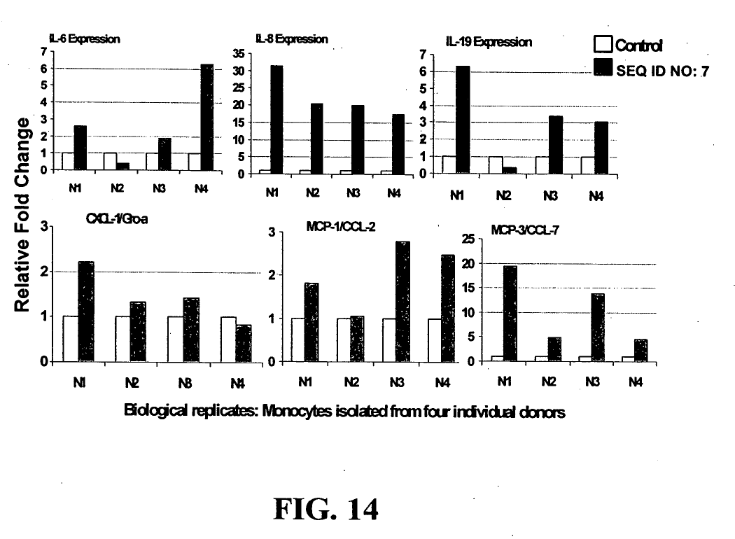

Transcriptional analysis of responses induced by the synthetic peptide using qPCR showed that the peptide was able to induce the expression of chemokines and interleukins such as MCP-3, MCP-1, CXCL-1, and IL-6, IL-19 (IL-10 superfamily) and IL-8, all of which are critical in cell recruitment and movement (FIG. 14). rMCP-1 is known to protect against Salmonella and Pseudomonas infections when given 6 hours prior to the initiation of infection in mice (Infection and Immunity 62:377-383, 1994), and to protect against acute septic peritonitis sue to bowel perforation (Journal of Immunology 163: 6148-54, 1999).

The peptide (SEQ ID NO: 7), as well as LL-37, also induced the expression of IL-19 (FIG. 14) belonging to the IL-1 0 superfamily. It is well known that IL-10 activates the JAK-STAT pathway leading to the up-regulation of the SOCS family members, all of these responses are required to potentially counteract pro-inflammatory responses. Indeed recombinant IL-10 is being considered as a therapeutic for rheumatoid arthritis (Rheum Dis Clin North Am. 24(3):629-39, 1998) and IL-10 protects against endotoxemia (Journal of Experimental Medicine 177:1205-8, 1993) and Pseudomonas-mediated gut-derived sepsis Antimicrobial Agents and Chemotherapy 42:2853-7, 1998).

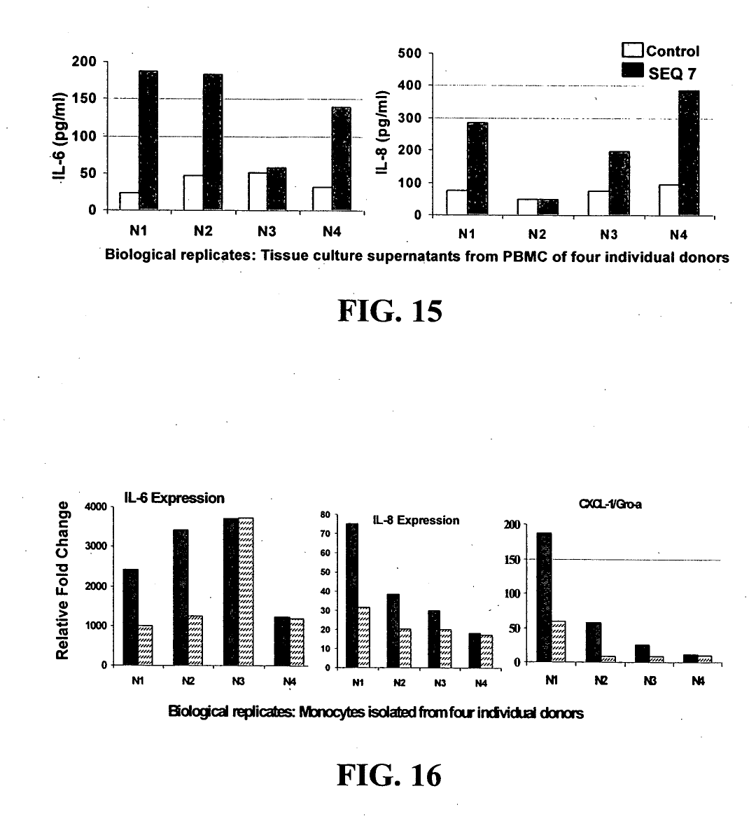

These genes are thus markers for the biological effects of SEQ ID NO: 7. As JAK-STAT signaling is mediated by members of the IL-10 superfamily, the observation that the synthetic peptide up-regulates the expression of IL-19 further supports the conclusion that one of the signal transduction pathways activated by host defense peptides is the JAK-STAT pathway, resulting in the up-regulation of genes in the SOCS family. The peptide also appears to regulate the NFκB pathway which can in addition lead to the expression of some of the members of the SOCS family. Thus there appears to be subtle changes in the regulation of critical pathways in immune responses (JAK-STAT, NFκB and MAPK) in the presence of the peptide which potentially suggests the mechanism by which the peptide functions in combating pathogenesis. Further analysis of protein production induced by the peptide in human PBMC by ELISA confirmed that SEQ ID NO: 7 is able to induce the secretion of interleukins such as IL-6 and IL-8 in human PBMC within 4 hours of stimulation, but did not induce the expression of the pro-inflammatory cytokine TNFα (FIG. 15). It is thus evident that the peptides induces the expression of critical immunity genes such as chemokines, interleukins, adhesion molecules and transcription factors that are able to modulate specific host defense mechanisms, and thereby prove to be beneficial therapeutics.

In response to bacterial endotoxin (LPS) there were 836 differentially expressed genes in human monocytes (Table 70), and 1012 genes were differentially expressed in response to LPS in the presence of the peptide SEQ ID NO: 7 (Table 71). The peptide appeared to induce the expression of several genes synergistically only in the presence of LPS. These genes are either induced only in the presence of the peptide on LPS stimulation (not with LPS alone) and/or the LPS-induced expression is synergistically enhanced in the presence of the peptide, but not with the peptide alone. These genes included several genes that are critical in immune response in presence of pathogenic challenge such as chemotactic factor CCL20, and CCL23; cytokine receptor EBI3, factors involved in lymphocyte activation such as SLAMF1, CD58, and IL32; regulators of signal transduction such as MAP2K2 (activator of ERK1), DUSP5 (ERK phopsphatase), MAPK8IP3, RIN2 (RAS-effector), RANBP9 (GTP-binding protein that affects Ras-signaling pathway), IP3 3-kinase A,-B3ATF, IRAK3, NM1 (augments cytokine-mediated STAT transcription), SP3, RAP2C, PNRC1, NEK1, CHC1, ZNF219, ZNF593, WIF1, PIM2, CD79A, and LATS2; substrate transporters such as SLC23A3, and SLC17A5; apoptosis regulators such as BOK (Bc1-2 like inhibitor), BIRC3, TNFRSF6, and CASP9; genes associated with plasma membrane such as STIM1, BPAG1, PTPN4, TRIM36, SDK1, and FNDC5 (fibronectin type III); genes involved in selective ion transport and in mediating selective ion-channel such as VGCNL1, TRPC5, CACNA1B, KCNA6, KCNJ2, KCNA10, and AQP9; growth modulating genes and/or those that play a role in wound healing such as FGF10, and AREG; inflammatory mediators such as PTGS2, SOD2, TNFAIP8 and anti-inflammatory gene TNIP3. This indicates that one of the mechanism by which cationic host defense peptides exhibit their protective mechanism is by delicately modulating specific signal pathways only in presence of agonists such as bacterial endotoxin, thus trigger just sufficient amount of inflammation and/or immune responses that is necessary to combat pathogenesis, while also maintaining the anti-inflammatory checks in place in order to prevent excessive inflammation that can lead to sepsis.

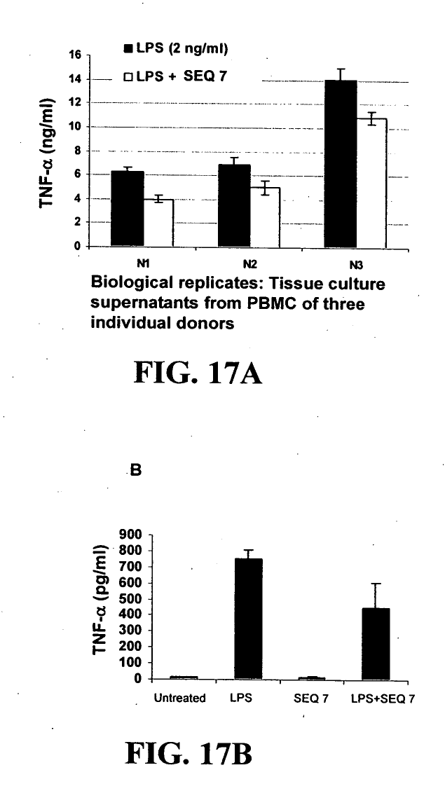

Of the 596 genes that were up-regulated by LPS stimulation, 33 genes were suppressed in the presence of the peptide (Table 72), which included the genes for pro-inflammatory TNF-α and NF-κB2 (p52). Interestingly, the peptide alone was able to up-regulate NF-κB2 as analyzed by DNA microarrays, an observation that also has mechanistic implications as individual subunits of NFκB are known to be involved in expression of only a subset of NFκB-regulated genes (the major pair of NFκB subunits p50 and p65 are responsible for much of the pro-inflammatory gene expression). Further transcriptional analysis using qRT-PCR revealed that the peptide exhibited the ability to suppress LPS-induced gene expression of cytokines such as IL-8, IL-6 as well as chemokines such as CXCL-1 within 4 hours of stimulation (FIG. 16). In addition, secretion of LPS-induced pro-inflammatory cytokine TNF-α was suppressed between 30-40% in the presence of peptide SEQ ID NO: 7 in human PBMC (FIG. 17A), as well as in human monocytic cell line THP-1 cells (FIG. 17B). Together, these results suggest that the synthetic peptide not only has the ability to suppress pro-inflammatory responses, e.g. TNF-α induced by LPS, similar to the natural human peptide LL-37 (Mookherjee, et al.), but also may have the potential to activate certain members of the NF-κB transcription factors such as NF-κB2 unlike the natural human peptide

TABLE 70
Gene profiling of differentially expressed genes in human monocytes due to
bacterial endotoxin (LPS) revealing 836 differentially expressed genes

| | | Fold change | |
|---|---|---|---|
| Gene Name | Gene Description | LPS | p-student |
| GPD1 | Glycerol-3-phosphate dehydrogenase [NAD+], | 103.4 | 0.06 |
| | cytoplasmic; GPD-C; GPDH-C | | |
| Q8NI35 | ATP/GTP-binding site motif A (P-loop); | 78.4 | 0.06 |
| | PDZ/DHR/GLGF domain | | |
| Q86Y93 | ATP/GTP-binding site motif A (P-loop); CSL zinc | 67.9 | 0.05 |
| | finger; Guanylate-binding protein; | | |
| KCNK6 | Exhibits outward rectification in a physiological K(+) | 61.5 | 0.04 |
| | gradient and mild inward rectification in symmetrical | | |
| | K(+) conditions | | |
| RHBDF1 | Rhomboid-like protein | 57.3 | 0.05 |
| FEZ2 | Involved in axonal outgrowth and fasciculation | 45.9 | 0.05 |
| O43348 | Argininosuccinate synthase | 43.3 | 0.03 |
| Q9C098 | Protein kinase; Serine/Threonine protein kinase | 43.1 | 0.04 |
| SMF | SMF protein KIAA0194 | 41.7 | 0.04 |
| WBSCR18 | Williams-Beuren syndrome chromosome region 18 | 36.3 | 0.05 |
| | protein | | |
| NRXN1 | Neuronal cell surface protein that may be involved in cell | 35.4 | 0.05 |
| | recognition and cell adhesion. May mediate intracellular | | |
| | signaling | | |
| DLX5 | Homeobox protein DLX-5 | 34.4 | 0.04 |
| BNIP1 | Implicated in the suppression of cell death. Interacts with | 34.1 | 0.04 |
| | the BCL-2 and adenovirus E1B 19 kDa proteins | | |
| Q9P175 | unknown | 33.6 | 0.03 |
| SMURF2 | E3 ubiquitin-protein ligase which accepts ubiquitin from | 32.7 | 0.05 |
| | an E2 ubiquitin-conjugating enzyme in the form of a | | |
| | thioester and then directly transfers it to targeted | | |
| | substrates SMAD1; SMAD2 and SMAD7 to trigger their | | |
| | ubiquitination and degradation. | | |
| C6orf74 | Metal-dependent phosphohydrolase, HD region | 32.6 | 0.06 |
| PLCG1 | PLC-gamma is a major substrate for heparin-binding | 32.6 | 0.04 |
| | growth factor 1 (acidic fibroblast growth factor)- | | |
| | activated tyrosine kinase | | |
| LRMP | Lymphoid-restricted membrane protein. | 32.0 | 0.05 |
| CD226 | Immunoglobulin-like | 31.1 | 0.04 |
| Q9Y4T9 | low complexity | 30.0 | 0.05 |
| Q8WUC6 | Bipartite nuclear localization signal; Class I peptide | 29.0 | 0.05 |
| | chain release factor domain | | |
| Q9P278 | low complexity | 28.6 | 0.03 |
| MCART1 | Mitochondrial carrier triple repeat 1 | 27.6 | 0.05 |
| DKC1 | Required for ribosome biogenesis and telomere | 24.2 | 0.03 |
| | maintenance. | | |
| ENSG00000162701 | DENN (AEX-3) domain; uDENN domain | 24.1 | 0.05 |
| TNC | SAM (substrate-adhesion molecule) that appears to | 23.6 | 0.04 |
| | inhibit cell migration. Ligand for integrins. | | |
| TRPM8 | Ion transport protein | 21.8 | 0.05 |
| SBNO1 | Helicase, C-terminal; RNA-binding region RNP-1 (RNA | 21.5 | 0.06 |
| | recognition motif) | | |
| Q7RTU0 | Basic helix-loop-helix dimerization domain bHLH | 21.4 | 0.03 |
| CHRNA1 | After binding acetylcholine, the AChR responds by an | 21.4 | 0.03 |
| | extensive change in conformation that affects all subunits | | |
| | and leads to opening of an ion-conducting channel across | | |
| | the plasma membrane | | |
| KIAA1529 | coiled-coil; low complexity; transmembrane | 21.3 | 0.06 |
| ALDOB | Fructose-bisphosphate aldolase B; Liver-type aldolase | 21.0 | 0.03 |
| Q8NHU6 | Bipartite nuclear localization signal; Maternal tudor | 20.9 | 0.04 |
| | protein | | |
| Q8N6Q6 | unknown | 20.3 | 0.01 |
| Q9H5P1 | Zn-finger, C-x8-C-x5-C-x3-H type | 19.9 | 0.06 |
| NR2F2 | Regulation of the apolipoprotein A-I gene transcription. | 19.0 | 0.05 |
| | Binds to DNA site A | | |
| SNX13 | May be involved in several stages of intracellular | 18.9 | 0.06 |
| | trafficking. Act as a GAP for Galphas | | |
| TULP2 | Tubby related protein 2; Tubby-like protein 2 | 18.9 | 0.05 |
| Q96HJ9; | unknown | 18.0 | 0.04 |
| BLZF1 | basic leucine zipper nuclear factor 1. | 17.7 | 0.04 |
| SLC35A4 | Nucleotide-sugar transporter | 17.1 | 0.04 |
| IDH3G | Isocitrate dehydrogenase [NAD] subunit gamma, | 17.0 | 0.05 |
| | mitochondrial precursor; Isocitric dehydrogenase; | | |
| | NAD(+)-specific ICDH | | |
| Q9BRK2 | Protein of unknown function DUF625 | 16.6 | 0.03 |
| RCL1 | Plays a role in 40S-ribosomal-subunit biogenesis in the | 16.2 | 0.00 |
| | early pre-rRNA processing steps at sites A0, A1 and A2 | | |
| | that are required for proper maturation of the 18S RNA | | |
| PMPCB | Cleaves presequences (transit peptides) from | 15.6 | 0.04 |
| | mitochondrial protein precursors | | |
| O43788; | NULL | 15.2 | 0.05 |
| GFM2; EFG2; | This protein promotes the GTP-dependent translocation | 15.1 | 0.06 |
| | of the nascent protein chain from the A-site to the P-site | | |
| | of the ribosome | | |
| SYT11 | May be involved in Ca(2+)-dependent exocytosis of | 14.8 | 0.03 |
| | secretory vesicles through Ca(2+) and phospholipid | | |
| | binding to the C2 domain or may serve as Ca(2+) sensors | | |
| | in the process of vesicular trafficking and exocytosis | | |
| C10orf11 | Leucine-rich repeat | 14.7 | 0.05 |
| FBN1 | Fibrillins are structural components of 10-12 nm | 14.6 | 0.05 |
| | extracellular calcium-binding microfibril. Fibrillin-1- | | |
| | containing microfibrils provide long-term force bearing | | |
| | structural support | | |
| Q9NVK9 | DH domain; Pleckstrin-like | 14.5 | 0.05 |
| MED6 | Plays a role in transcriptional coactivation | 14.2 | 0.05 |
| ENSG00000105849 | RNA polymerase Rpa43 subunit | 13.5 | 0.04 |
| SACM1L | Synaptojanin, N-terminal | 13.5 | 0.05 |
| SYNE1 | Involved in the maintenance of nuclear organization and | 13.4 | 0.06 |
| | structural integrity. Probable anchoring protein which | | |
| | tethers the nucleus to the cytoskeleton. | | |
| WNT5B | Ligand for members of the frizzled family of seven | 13.2 | 0.03 |
| | transmembrane receptors. Probable developmental | | |
| | protein. | | |
| DHX8 | Facilitates nuclear export of spliced mRNA by releasing | 13.0 | 0.05 |
| | the RNA from the spliceosome | | |
| PRO0461 | Protein PRO0461 | 11.6 | 0.03 |
| SIRT1 | NAD-dependent deacetylase, which regulates processes | 11.5 | 0.03 |
| | such as apoptosis and muscle differentiation by | | |
| | deacetylating key proteins, including p53, TAF1B, etc | | |
| NEK2 | Protein kinase that is involved in mitotic regulation. | 11.5 | 0.04 |
| MMP17 | Endopeptidase that degrades various components of the | 11.5 | 0.06 |
| | extracellular matrix, such as fibrin. May be involved in | | |
| | the activation of membrane-bound precursors of growth | | |
| | factors or inflammatory mediators, such as tumor | | |
| | necrosis factor-alpha. | | |
| TIMM9 | Likely to be involved in the import and insertion of | 11.4 | 0.06 |
| | hydrophobic membrane proteins into the mitochondrial | | |
| | inner membrane | | |
| ARRB1 | Regulates beta-adrenergic receptor function. Beta- | 11.1 | 0.05 |
| | arrestins seem to bind phosphorylated beta-adrenergic | | |
| | receptors, thereby causing a significant impairment of | | |
| | their capacity to activate G(S) proteins | | |
| O75872 | rab3 GTPase-activating protein, non-catalytic subunit. | 11.0 | 0.03 |
| Q96IZ9 | PTD016 protein. | 10.8 | 0.03 |
| POMT2 | Transfers mannosyl residues to the hydroxyl group of | 10.8 | 0.05 |
| | serine or threonine residues. | | |
| METTL3 | N6-methyltransferase that methylates adenosine residues | 10.7 | 0.05 |
| | of some mRNAs. | | |
| DUSP14 | Involved in the inactivation of MAP kinases. | 10.4 | 0.02 |
| | Dephosphorylates ERK, JNK and p38 MAP-kinases | | |
| SLC27A6 | AMP-dependent synthetase and ligase | 10.3 | 0.05 |
| PTPRCAP | Protein tyrosine phosphatase receptor type C-associated | 10.2 | 0.05 |
| | protein; CD45-associated protein; Lymphocyte | | |
| | phosphatase-associated phosphoprotein | | |
| OSBPL7 | Oxysterol binding protein-related protein 7; OSBP- | 10.2 | 0.01 |
| | related protein 7; ORP-7 | | |
| ZNF251 | ATP/GTP-binding site motif A (P-loop); KRAB box; | 10.2 | 0.05 |
| | Zn-finger, C2H2 subtype; | | |
| UBE2N | The UBE2V2/UBE2N heterodimer catalyzes the | 9.7 | 0.01 |
| | synthesis of non-canonical poly-ubiquitin chains that are | | |
| | linked through Lys-63. Mediates transcriptional | | |
| | activation of target genes. Plays a role in the control of | | |
| | progress through the cell cycle and differentiation. | | |
| TBCA | Tubulin-folding protein; involved in the early step of the | 9.6 | 0.04 |
| | tubulin folding pathway | | |
| Q96NS3 | low complexity; transmembrane | 9.6 | 0.05 |
| KHK | Ketohexokinase; Hepatic fructokinase | 9.5 | 0.04 |
| GSTZ1 | Bifunctional enzyme showing minimal glutathione- | 9.4 | 0.04 |
| | conjugating activity and low glutathione peroxidase | | |
| | activity | | |
| ANKRD5 | Ankyrin repeat domain protein 5 | 9.3 | 0.04 |
| UBXD2 | UBX domain-containing protein 2 | 9.3 | 0.04 |
| Q8N336; | Protein of unknown function DUF609 | 9.2 | 0.05 |
| ALOX5 | Arachidonate 5-lipoxygenase; 5-lipoxygenase; 5-LO | 9.2 | 0.05 |
| GPR174 | Putative receptor for purines coupled to G-proteins | 9.1 | 0.05 |
| TRIP4 | Transcription coactivator of nuclear receptors which | 9.0 | 0.05 |
| | functions in conjunction with CBP-p300 and SRC-1 and | | |
| | may play an important role in establishing distinct | | |
| | coactivator complexes under different cellular | | |
| | conditions. Plays a pivotal role in the transactivation of | | |
| | NF-kappa-B, SRF and AP1. Acts as a mediator of | | |
| | transrepression between nuclear receptor and AP1 or | | |
| | NFκ-B. | | |
| Q9P1G1 | signal peptide | 8.7 | 0.02 |
| Q96E44 | Beta and gamma crystallin; Nuclear protein SET | 8.5 | 0.02 |
| HCG9; HLA-J | Immunoglobulin-like; Immunoglobulin/major | 8.5 | 0.05 |
| | histocompatibility complex; Major histocompatibility | | |
| | complex protein, class I | | |
| Q9BUJ0 | Alpha/beta hydrolase; Esterase/lipase/thioesterase, active | 8.2 | 0.04 |
| | site | | |
| PRLR | Cytokine receptor, common beta/gamma chain; | 8.0 | 0.02 |
| | Fibronectin, type III; Long hematopoietin receptor, | | |
| | single chain | | |
| DACH2 | Bipartite nuclear localization signal; Transforming | 8.0 | 0.03 |
| | protein Ski | | |
| O60384 | Zn-finger, C2H2 type | 7.9 | 0.03 |
| Q9BYE9 | Cadherin | 7.9 | 0.05 |
| TOP3B | Possesses negatively supercoiled DNA relaxing activity | 7.4 | 0.06 |
| MPP4 | May play a role in retinal photoreceptors development | 7.3 | 0.05 |
| LNX | E3 Ubiquitin ligase protein that mediates ubiquitination | 7.3 | 0.04 |
| | and subsequent proteasomal degradation of NUMB. | | |
| CYB5M; | Cytochrome b5 is a membrane bound hemoprotein which | 7.2 | 0.05 |
| OMB5 | function as an electron carrier for several membrane | | |
| | bound oxygenases | | |
| GRTP1 | RabGAP/TBC domain; Somatotropin hormone | 7.1 | 0.03 |
| Q9NZ13 | Zn-finger, C2H2 type | 6.9 | 0.03 |
| PROZ | Appears to assist hemostasis by binding thrombin and | 6.9 | 0.05 |
| | promoting its association with phospholipid vesicles | | |
| HELIC1 | ATP/GTP-binding site motif A (P-loop); DEAD/DEAH | 6.8 | 0.04 |
| | box helicase; Helicase, C-terminal; Sec63 domain | | |
| EDG7 | Receptor for lysophosphatidic acid (LPA), a mediator of | 6.6 | 0.04 |
| | diverse cellular activities. May play a role in the | | |
| | development of ovarian cancer. Seems to be coupled to | | |
| | the G(i)/G(0) and G(q) families of heteromeric G | | |
| | proteins | | |
| EPM2A | Likely multifunctional endocytic receptor that may be | 6.6 | 0.04 |
| | implicated in the uptake of lipoproteins and of proteases. | | |
| | Binds LDL and receptor-associated protein (RAP). | | |
| | Could play a role in cell-cell interaction | | |
| Q9UPS4 | coiled-coil; low complexity | 6.5 | 0.05 |
| Q96LQ8 | Alpha-2-macroglobulin; Alpha-2-macroglobulin, N- | 6.5 | 0.04 |
| | terminal | | |
| MYL4 | Regulatory light chain of myosin. Does not bind calcium | 6.5 | 0.04 |
| ACTL6 | Required for maximal ATPase activity of BRG1 and are | 6.5 | 0.03 |
| | also required with BRG1 for association of the complex | | |
| | with chromatin/matrix | | |
| CHCHD5 | Bipartite nuclear localization signal | 6.4 | 0.03 |
| MFAP1 | Component of the elastin-associated microfibrils | 6.4 | 0.05 |
| PLAC8 | Placenta-specific gene 8 protein; C15 protein | 6.4 | 0.01 |
| LAMA3 | Laminin, binds to cells via a high affinity receptor is | 6.2 | 0.04 |
| | thought to mediate the attachment, migration and | | |
| | organization of cells into tissues during embryonic | | |
| | development by interacting with other extracellular | | |
| | matrix components | | |
| EDN1 | Endothelins are endothelium-derived vasoconstrictor | 6.2 | 0.05 |
| | peptides | | |
| MSX1 | Acts as a transcriptional repressor. May play a role in | 6.1 | 0.04 |
| | limb-pattern formation. Acts in cranofacial development | | |
| | and specifically in odontogenesis | | |
| PKP4 | May play a role in junctional plaques | 6.1 | 0.05 |
| PRY | Testis-specific PTP-BL related Y protein; PTPN13-like | 6.1 | 0.05 |
| | protein | | |
| Q13862 | DNA-binding protein. | 6.0 | 0.04 |
| PAK2 | The activated kinase acts on a variety of targets. | 6.0 | 0.03 |
| | Phosphorylates ribosomal protein S6, histone H4 and | | |
| | myelin basic protein | | |
| Wdr68 | WD-repeat protein 68; WD-repeat protein An11 | 5.9 | 0.02 |
| | homolog | | |
| SENP6 | Protease that releases SUMO-1 from its precursor | 5.9 | 0.03 |
| | sequence | | |
| DNMT2 | Its strong binding to DNA suggests that it may mark | 5.9 | 0.04 |
| | specific sequences in the genome by binding to DNA | | |
| | through the specific target-recognizing motif. Not active | | |
| | as a DNA methyltransferase. | | |
| IL8 | IL-8 is a chemotactic factor that attracts neutrophils, | 5.7 | 0.01 |
| | basophils, and T-cells, but not monocytes. It is also | | |
| | involved in neutrophil activation. It is released from | | |
| | several cell types in response to an inflammatory | | |
| | stimulus. | | |
| CCS | Delivers copper to copper zinc superoxide dismutase | 5.6 | 0.02 |
| | (SOD1) | | |
| RGS14 | Inhibits signal transduction by increasing the GTPase | 5.6 | 0.05 |
| | activity of G protein-α subunits resulting in their inactive | | |
| | GDP-bound form | | |
| ARHGAP20 | RA domain; RhoGAP domain | 5.6 | 0.05 |
| COLEC10 | C-type lectin; Collagen triple helix repeat | 5.6 | 0.02 |
| Q96PN6 | ATP/GTP-binding site motif A (P-loop); Guanylate | 5.2 | 0.05 |
| | cyclase | | |
| FBXW5 | Cyclin-like F-box; G-protein beta WD-40 repeat | 5.1 | 0.04 |
| ASTN2 | Fibronectin, type III | 5.1 | 0.03 |
| PCYT1B | Controls phosphatidylcholine synthesis | 5.0 | 0.06 |
| MUC11 | Actin-binding, actinin-type; Eukaryotic RNA polymerase | 4.9 | 0.04 |
| | II heptapeptide repeat | | |
| Q8NC34 | Immunoglobulin-like | 4.9 | 0.06 |
| Q9UFY9 | ATP/GTP-binding site motif A (P-loop); Cell | 4.9 | 0.03 |
| | division/GTP binding protein | | |
| APLP1 | May play a role in postsynaptic function. The C-terminal | 4.8 | 0.05 |
| | gamma-secretase processed fragment, ALID1, activates | | |
| | transcription activation through APBB1 (Fe65) binding. | | |
| | May interact with cellular G-protein signaling pathways. | | |
| Q96N54 | Olfactory receptor; Rhodopsin-like GPCR superfamily | 4.8 | 0.02 |
| O60290 | KRAB box | 4.8 | 0.05 |
| COL5A1 | Collagen triple helix repeat; Fibrillar collagen, C- | 4.8 | 0.02 |
| | terminal; Laminin G; Prenyl group binding site (CAAX | | |
| | box) | | |
| NRG1 | Direct ligand for ERBB3 and ERBB4 tyrosine kinase | 4.7 | 0.05 |
| | receptors. Concomitantly recruits coreceptors, resulting | | |
| | in ligand-stimulated tyrosine phosphorylation and | | |
| | activation of the ERBB receptors. Multiple isoforms | | |
| | perform diverse functions such as inducing growth and | | |
| | differentiation of epithelial, glial, neuronal, and skeletal | | |
| | muscle cells. | | |
| Q96FB5 | CGI-41 protein | 4.6 | 0.02 |
| CYP2J2 | This enzyme metabolizes arachidonic acid | 4.6 | 0.06 |
| | predominantly via a NADPH-dependent olefin | | |
| | epoxidation to all four regioisomeric cis- | | |
| | epoxyeicosatrienoic acids. | | |
| DBI | Binds medium-and long-chain acyl-CoA esters with | 4.6 | 0.02 |
| | very high affinity and may function as an intracellular | | |
| | carrier of acyl-CoA esters. This protein may also act as a | | |
| | neuropeptide to modulate the action of the GABA | | |
| | receptor | | |
| HOOK1 | Cytoskeletal linker protein, which may be involved in | 4.6 | 0.04 |
| | tethering membrane bound organelles to the | | |
| | cytoskeleton. | | |
| FTCD | Folate-dependent enzyme, that displays both transferase | 4.6 | 0.03 |
| | and deaminase activity. Serves to channel one-carbon | | |
| | units from formiminoglutamate to the folate pool | | |
| CD84 | Leukocyte antigen CD84. | 4.5 | 0.04 |
| HIPK2 | Protein kinase acting as a corepressor of several | 4.5 | 0.02 |
| | transcription factors, including SMAD1 and | | |
| | POU4F1/Brn3a and probably NK homeodomain | | |
| | transcription factors: Inhibits cell growth and promotes | | |
| | apoptosis. In response to TGFB, cooperates with DAXX | | |
| | to activate JNK. Phosphorylates the antiapoptotic factor | | |
| | CTBP1 and promotes its proteasomal degradation. In the | | |
| | Wnt/beta-catenin signaling pathway acts as an | | |
| | intermediate kinase between TAK1 and NLK to promote | | |
| | the proteasomal degradation of c-Myb | | |
| Q8TDS9 | putative G-protein coupled receptor GPCR42. | 4.4 | 0.05 |
| Q9H7B7 | low complexity; signal peptide | 4.4 | 0.05 |
| CCL20 | Chemotactic factor that attracts lymphocytes and, | 4.4 | 0.01 |
| | slightly, neutrophils, but not monocytes. Inhibits | | |
| | proliferation of myeloid progenitors in colony formation | | |
| | assays. | | |
| NFKB2 | NFκB subunit; p52 and p100 are respectively the minor | 4.3 | 0.02 |
| | and major form. Appears to have dual functions such as | | |
| | cytoplasmic retention of attached NFκB proteins and | | |
| | generation of p52 by a cotranslational proteasome- | | |
| | mediated processing. p52 binds to the κB consensus | | |
| | sequence “GGRNNYYCC-3”, located in the enhancer | | |
| | region of genes involved in immune response and acute | | |
| | phase reactions. | | |
| C13orf11 | coiled-coil; low complexity; signal peptide; | 4.3 | 0.06 |
| | transmembrane | | |
| MKKS | May play a role in protein processing in limb, cardiac | 4.3 | 0.06 |
| | and reproductive system development | | |
| SLC6A1 | Terminates the action of GABA by its high affinity | 4.2 | 0.02 |
| | sodium-dependent reuptake into presynaptic terminals | | |
| BAZ2A | May play a role in transcriptional regulation interacting | 4.2 | 0.01 |
| | with ISWI. May serve a specific role in maintaining or | | |
| | altering the chromatin structure of the rDNA locus | | |
| SLC16A10 | T-type amino acid transporter 1; solute carrier family 16, | 4.2 | 0.05 |
| | # 10 | | |
| Q8NG48 | WINS1 protein isoform 1. | 4.2 | 0.05 |
| ARHGEF1 | Seems to play a role in the regulation of RhoA GTPase | 4.2 | 0.00 |
| | by guanine nucleotide-binding α-12 andα-13 subunits. | | |
| | GTPase-activating protein (GAP) for these subunits, and | | |
| | as guanine nucleotide exchange factor (GEF) for RhoA | | |
| | GTPase; stimulates the RhoGEF activity. | | |
| COL9A2 | Structural component of hyaline cartilage and vitreous of | 4.1 | 0.05 |
| | the eye | | |
| CTLA4 | Possibly involved in T-cell activation. Binds to B7-1 | 4.1 | 0.02 |
| | (CD80) and B7-2 (CD86) | | |
| SYNGR2 | Synaptogyrin-2; Cellugyrin | 4.1 | 0.05 |
| PSMA1 | Proteasome, a multicatalytic proteinase complex with an | 4.1 | 0.04 |
| | ATP-dependent proteolytic activity | | |
| EPN1 | Binds to membranes enriched in phosphatidylinositol- | 4.1 | 0.05 |
| | 4,5-biphosphate. Modifies membrane curvature, | | |
| | facilitates the formation of clathrin-coated invaginations, | | |
| | regulates receptor-mediated endocytosis | | |
| TMSB10 | Important role in the organization of the cytoskeleton. | 4.0 | 0.06 |
| | Binds to and sequesters actin monomers (G actin) and | | |
| | therefore inhibits actin polymerization | | |
| TCN1 | Vitamin B12-binding protein. Transports cobalamin into | 4.0 | 0.03 |
| | cells | | |
| RBM3 | Putative RNA-binding protein 3; RNA-binding motif | 4.0 | 0.01 |
| | protein 3 | | |
| C20orf26 | Protein C20orf26 | 4.0 | 0.06 |
| HYAL4 | EGF-like domain; Glycoside hydrolase, family 56; | 3.8 | 0.05 |
| | Glycoside hydrolase, family 56, sperm surface protein | | |
| | PH20; Multicopper oxidase, type 1 | | |
| BACH1 | Transcriptional regulator that acts as repressor or | 3.8 | 0.04 |
| | activator. Binds, in-vitro, to NF-E2 binding sites. Play | | |
| | important roles in coordinating transcription activation | | |
| | and repression by MAFK | | |
| PMAIP1 | Phorbol-12-myristate-13-acetate-induced protein 1; | 3.8 | 0.02 |
| Q96T82 | signal peptide; transmembrane | 3.7 | 0.00 |
| TRAC | T-cell receptor alpha chain C region | 3.7 | 0.04 |
| KLF8 | Transcriptional repressor. Binds to CACCC-box | 3.7 | 0.04 |
| | promoter elements | | |
| DVL2 | May play a role in the signal transduction pathway | 3.7 | 0.02 |
| | mediated by multiple Wnt genes | | |
| CRK7 | Cell division cycle 2-related protein kinase 7 | 3.7 | 0.04 |
| Q9H631 | Mak10 subunit, NatC N(alpha)-terminal | 3.6 | 0.05 |
| | acetyltransferase | | |
| ZNF208 | KRAB box; Neutral zinc metallopeptidases; Zn-finger, | 3.6 | 0.05 |
| | C2H2 subtype; | | |
| MAP2K4 | Dual specificity kinase that activates the JUN kinases | 3.6 | 0.05 |
| | MAPK8 (JNK1) and MAPK9 (JNK2) as well as | | |
| | MAPK14 (p38) but not MAPK1 (ERK2) or MAPK3 | | |
| | (ERK1) | | |
| CENTB2 | GTPase-activating protein for the ADP ribosylation | 3.6 | 0.03 |
| | factor family | | |
| Q8N958 | unknown | 3.6 | 0.03 |
| Q9BYA6 | low complexity | 3.5 | 0.04 |
| ZFYVE20 | Zn-finger, C2H2 type; Zn-finger, FYVE type | 3.5 | 0.02 |
| DSTN | Actin-depolymerizing protein. Severs F-actin filaments | 3.5 | 0.02 |
| | and binds to actin monomers (G-actin), in pH- | | |
| | independent manner | | |
| ITGB4 | Integrin alpha-6/beta-4 is a receptor for laminin. It plays | 3.5 | 0.03 |
| | a critical structural role in the hemidesmosome of | | |
| | epithelial cells | | |
| M6PR | Transport of phosphorylated lysosomal enzymes from | 3.5 | 0.05 |
| | the Golgi complex and the cell surface to lysosomes. | | |
| NEF3 | Neurofilaments usually contain 3 intermediate filament | 3.4 | 0.01 |
| | proteins: L, M, and H involved in maintenance of | | |
| | neuronal caliber | | |
| Q8N4T8 | 2,3-dihydro-2,3-dihydroxybenzoate dehydrogenase; | 3.4 | 0.03 |
| | Glucose/ribitol dehydrogenase; | | |
| ZNF36 | May be involved in transcriptional regulation | 3.4 | 0.02 |
| CCL2 | Chemotactic factor that attracts monocytes and basophils | 3.4 | 0.02 |
| | but not neutrophils or eosinophils. Has been implicated | | |
| | in the pathogenesis of diseases characterized by | | |
| | monocytic infiltrates, like psoriasis, rheumatoid arthritis | | |
| | or atherosclerosis. May be involved in the recruitment of | | |
| | monocytes into the arterial wall during the disease | | |
| | process of atherosclerosis | | |
| ANKMY1 | Ankyrin repeat and MYND domain protein 1; Testis- | 3.4 | 0.05 |
| | specific ankyrin-like protein 1; Zinc-finger MYND | | |
| | domain protein 13 | | |
| ABCC13 | Putative ATP-binding cassette transporter C13 | 3.4 | 0.04 |
| Q86TW0 | Bipartite nuclear localization signal; Zn-finger, C-x8-C- | 3.4 | 0.03 |
| | x5-C-x3-H type | | |
| ZNF213 | May be involved in transcriptional regulation | 3.4 | 0.04 |
| HNF4A | Transcriptionally controlled transcription factor. Binds to | 3.3 | 0.01 |
| | DNA sites required for the transcription of alpha 1- | | |
| | antitrypsin, apolipoprotein CIII, transthyretin genes and | | |
| | HNF1-alpha. | | |
| MAFF | Interacts with the upstream promoter region of the | 3.3 | 0.01 |
| | oxytocin receptor gene. May be a transcriptional | | |
| | enhancer. May also serve as transcriptional activator by | | |
| | dimerizing with other basic-zipper proteins and | | |
| | recruiting them to specific DNA-binding sites. May be | | |
| | involved in the cellular stress response | | |
| FBXO32 | Probably recognizes and binds to some phosphorylated | 3.3 | 0.03 |
| | proteins and promotes their ubiquitination and | | |
| | degradation during skeletal muscle atrophy | | |
| Q9P233 | Calponin-like actin-binding; Eggshell protein; Leucine- | 3.3 | 0.05 |
| | rich repeat | | |
| TNF | Pro-inflammatory cytokine tumour necrosis factor α that | 3.3 | 0.02 |
| | binds to TNFRSF1A/TNFR1 and TNFRSF1B/TNFBR. | | |
| CXCL2 | Hematoregulatory chemokine, which, in vitro, suppresses | 3.3 | 0.00 |
| | hematopoietic progenitor cell proliferation. Produced by | | |
| | activated monocytes & neutrophils and expressed at sites | | |
| | of inflammation. | | |
| Q8N233 | NHL repeat; Zn-finger, C2H2 type | 3.3 | 0.03 |
| MAK | Could play an important function in spermatogenesis | 3.3 | 0.03 |
| GPR30 | Orphan receptor; possibly for a chemokine | 3.2 | 0.01 |
| MAGMAS | Mitochondria-associated granulocyte macrophage CSF | 3.2 | 0.05 |
| | signaling molecule, mitochondrial precursor | | |
| LHX2 | Transcriptional regulatory protein involved in the control | 3.2 | 0.05 |
| | of cell differentiation in developing lymphoid and neural | | |
| | cell types | | |
| Q96LP3 | Leucine-rich repeat | 3.2 | 0.05 |
| Q9NXD2 | Bipartite nuclear localization signal | 3.2 | 0.02 |
| FOXQ1 | Forkhead box protein Q1; Hepatocyte nuclear factor 3 | 3.2 | 0.01 |
| | forkhead homolog 1; | | |
| TFEC | Basic helix-loop-helix dimerization domain bHLH | 3.1 | 0.03 |
| Q96EC8 | Protein of unknown function DUF649 | 3.1 | 0.03 |
| PTGS2 | May have a role as a major mediator of inflammation | 3.1 | 0.01 |
| | and/or a role for prostanoid signaling in activity- | | |
| | dependent plasticity | | |
| Q8N3K5 | Cysteine-rich flanking region, N-terminal; | 3.1 | 0.02 |
| | Immunoglobulin-like; Leucine-rich repeat; RNA-binding | | |
| | region RNP-1 | | |
| DNAH5 | ATP/GTP-binding site motif A (P-loop); Dynein heavy | 3.0 | 0.01 |
| | chain; Eukaryotic thiol (cysteine) protease | | |
| HDC | Histidine decarboxylase; HDC | 3.0 | 0.00 |
| C6orf149 | Bipartite nuclear localization signal; Complex 1 LYR | 3.0 | 0.06 |
| | protein | | |
| PTGIS | Catalyzes the isomerization of prostaglandin H2 to | 3.0 | 0.06 |
| | prostacyclin | | |
| IL6 | IL-6 is a cytokine with a wide variety of biological | 2.9 | 0.00 |
| | functions: it plays an essential role in the final | | |
| | differentiation of B-cells into Ig-secreting cells, it | | |
| | induces myeloma and plasmacytoma growth, it induces | | |
| | nerve cells differentiation, in hepatocytes it induces acute | | |
| | phase reactants | | |
| CXCL1 | Has chemotactic activity for neutrophils. May play a role | 2.9 | 0.01 |
| | in inflammation and exerts its effects on endothelial cells | | |
| | in an autocrine fashion. | | |
| KPNB1 | Functions in nuclear protein import, either in association | 2.9 | 0.05 |
| | with an adapter protein, like an importin-alpha subunit, | | |
| | which binds to nuclear localization signals (NLS) in | | |
| | cargo substrates, or by acting as autonomous nuclear | | |
| | transport receptor. | | |
| CD81 | May play an important role in the regulation of | 2.8 | 0.05 |
| | lymphoma cell growth. May acts as the viral receptor for | | |
| | HCV | | |
| GNS | N-acetylglucosamine-6-sulfatase precursor; | 2.8 | 0.03 |
| PTS | Involved in the biosynthesis of tetrahydrobiopterin, an | 2.8 | 0.01 |
| | essential cofactor of aromatic amino acid hydroxylases. | | |
| MYLIP | Band 4.1 domain; Ezrin/radixin/moesin ERM | 2.8 | 0.01 |
| Q96IT8 | low complexity | 2.8 | 0.02 |
| SLC17A4 | Na/PO4 cotransporter; solute carrier family 17, member 4 | 2.8 | 0.02 |
| NPAS2 | Neuronal PAS domain protein 2; MOP4 | 2.8 | 0.00 |
| PANK3 | Plays a role in the physiological regulation of the | 2.8 | 0.00 |
| | intracellular CoA concentration | | |
| PHLDA2 | Pleckstrin-like | 2.8 | 0.02 |
| Q96MX1 | down-regulated by Ctnnb1, a. | 2.8 | 0.06 |
| SP3 | Binds to GT and GC boxes promoters elements. Probable | 2.8 | 0.06 |
| | transcriptional activator | | |
| Q9Y3U6 | low complexity | 2.7 | 0.01 |
| NRP1 | The membrane-bound isoform 1 is a receptor involved in | 2.7 | 0.04 |
| | the development of the cardiovascular system, in VEGF- | | |
| | induced angiogenesis, in the formation of certain | | |
| | neuronal circuits and in organogenesis outside the | | |
| | nervous system. | | |
| NCK1 | Adapter protein which associates with tyrosine- | 2.7 | 0.03 |
| | phosphorylated growth factor receptors or their cellular | | |
| | substrates | | |
| Q8WUC7 | Bipartite nuclear localization signal | 2.7 | 0.02 |
| Q9P2X3 | Protein of unknown function UPF0029 | 2.7 | 0.01 |
| NICE1 | NICE-1 protein | 2.7 | 0.06 |
| SH3YL1 | Protein of unknown function DUF500; SH3 domain | 2.7 | 0.01 |
| CCL23 | Shows chemotactic activity for monocytes, resting T- | 2.7 | 0.03 |
| | lymphocytes, and neutrophils, but not for activated | | |
| | lymphocytes. | | |
| SNAPAP | May modulate a step between vesicle priming, fusion | 2.7 | 0.05 |
| | and calcium-dependent neurotransmitter release. Its | | |
| | phosphorylation state influences exocytotic protein | | |
| | interactions and may regulate synaptic vesicle | | |
| | exocytosis. May also have role in SNARE-mediated | | |
| | membrane fusion in non-neuronal cells | | |
| ZHX2 | Homeobox; Zn-finger, C2H2 type | 2.6 | 0.03 |
| LOXL2 | Lysyl oxidase homolog 2 precursor; Lysyl oxidase- | 2.6 | 0.01 |
| | related protein WS9-14 | | |
| ACSL6 | Activation of long-chain fatty acids for both synthesis of | 2.6 | 0.05 |
| | cellular lipids, and degradation via beta-oxidation. Plays | | |
| | an important role in fatty acid metabolism in brain | | |
| Q96BW9 | unknown | 2.6 | 0.05 |
| Q9BT00 | RPA interacting protein | 2.6 | 0.03 |
| Q9UJA5 | Bipartite nuclear localization signal; Eukaryotic initiation | 2.6 | 0.03 |
| | factor 3, gamma subunit | | |
| ICAM3 | ICAM proteins are ligands for the leukocyte adhesion | 2.6 | 0.04 |
| | LFA-1 protein (integrin alpha-L/beta-2). ICAM3 is also a | | |
| | ligand for integrin alpha-D/beta-2 | | |
| TENC1 | Bipartite nuclear localization signal; Protein kinase C, | 2.6 | 0.01 |
| | phorbol ester/diacylglycerol binding; SH2 motif | | |
| NME1 | Major role in the synthesis of nucleoside triphosphates | 2.5 | 0.02 |
| | other than ATP | | |
| IL1F9 | Function as an agonist of NFκB activation through the | 2.5 | 0.04 |
| | orphan IL-1-receptor-related protein 2. Could constitute | | |
| | part of an independent signaling system analogous to | | |
| | interleukin-1α, and β receptor agonist and interleukin-1 | | |
| | receptor type I (IL-1R1), that is present in epithelial | | |
| | barriers and takes part in local inflammatory response | | |
| Q8WVV9 | RNA-binding region RNP-1; Serine/threonine | 2.5 | 0.05 |
| | dehydratase, pyridoxal-phosphate attachment site | | |
| ZNF177 | May be involved in transcriptional regulation | 2.5 | 0.02 |
| DNASE1 | Seems to be involved in cell death by apoptosis. Binds | 2.5 | 0.01 |
| | specifically to G-actin and blocks actin polymerization | | |
| Q8N867 | unknown | 2.5 | 0.04 |
| SOD2 | Destroys radicals which are normally produced within | 2.5 | 0.01 |
| | the cells and which are toxic to biological systems | | |
| Q9H9C7 | BRCT domain | 2.5 | 0.04 |
| C16orf3 | Protein C16orf3 | 2.5 | 0.02 |
| Q9BRJ9 | Basic helix-loop-helix dimerization domain bHLH | 2.5 | 0.04 |
| GDA | Catalyzes the hydrolytic deamination of guanine, | 2.5 | 0.01 |
| | producing xanthine and ammonia | | |
| LILRB1 | Receptor for class I MHC antigens. Recognizes a broad | 2.5 | 0.05 |
| | spectrum of HLA-A, HLA-B, HLA-C and HLA-G | | |
| | alleles. Ligand binding results in inhibitory signals and | | |
| | down-regulation of the immune response. Engagement of | | |
| | LILRB1 present on NK cells or T-cells by class I MHC | | |
| | molecules protects the target cells from lysis. | | |
| HBG1 | The epsilon chain is a beta-type chain of early | 2.5 | 0.04 |
| | mammalian embryonic hemoglobin | | |
| MAP3K8 | Able to activate NFκB 1 by stimulating proteasome- | 2.5 | 0.06 |
| | mediated proteolysis of NFκB 1/p105. Plays a role in the | | |
| | cell cycle. | | |
| Q8WVI0 | low complexity | 2.5 | 0.02 |
| HELLS | Bipartite nuclear localization signal; Helicase, C- | 2.5 | 0.02 |
| | terminal; SNF2 related domain | | |
| CDK11 | Protein kinase; Serine/Threonine protein kinase | 2.4 | 0.05 |
| ARHGDIA | Regulates the GDP/GTP exchange reaction of the Rho | 2.4 | 0.02 |
| | proteins by inhibiting GDP dissociation and subsequent | | |
| | GTP binding | | |
| Q9H759 | Immunoglobulin-like | 2.4 | 0.04 |
| SERPINB8 | Inhibits urokinase-type plasminogen activator. The | 2.4 | 0.04 |
| | monocyte derived PAI-2 is distinct from the endothelial | | |
| | cell-derived PAI-1 | | |
| Q86VU9 | Proline-rich region | 2.4 | 0.00 |
| Q8IUZ5 | Aminotransferase class-III | 2.4 | 0.05 |
| Q9BWJ2 | unknown | 2.4 | 0.05 |
| ARG2 | May play a role in the regulation of extra-urea cycle | 2.4 | 0.04 |
| | arginine metabolism and also in down-regulation of | | |
| | nitric oxide synthesis. | | |
| TNFAIP6 | Possibly involved in cell-cell and cell-matrix interactions | 2.4 | 0.01 |
| | during inflammation and tumorigenesis | | |
| C14orf132 | transmembrane | 2.4 | 0.05 |
| RIPK2 | Activates pro-caspase-1 and pro-caspase-8. Potentiates | 2.4 | 0.01 |
| | CASP-8-mediated apoptosis. Activates NF-kappa-B | | |
| SLC39A8 | Zinc transporter ZIP | 2.4 | 0.02 |
| ATP2B1 | This magnesium-dependent enzyme catalyzes the | 2.3 | 0.01 |
| | hydrolysis of ATP coupled with the transport of calcium | | |
| | out of the cell | | |
| CRKL | May mediate the transduction of intracellular signals | 2.3 | 0.02 |
| PABPC3 | Binds the poly(A) tail of mRNA. May be involved in | 2.3 | 0.05 |
| | cytoplasmic regulatory processes of mRNA metabolism. | | |
| ERCC6 | Is involved in the preferential repair of active genes. | 2.3 | 0.06 |
| | Presumed DNA or RNA unwinding function. | | |
| Q8NC30 | transmembrane | 2.3 | 0.00 |
| Q969W3 | low complexity | 2.3 | 0.00 |
| HTR1F | One of the several different receptors for serotonin, a | 2.3 | 0.01 |
| | biogenic hormone that functions as a neurotransmitter, a | | |
| | hormone, and a mitogen. Activity mediated by G | | |
| | proteins that inhibit adenylate cyclase activity | | |
| JARID1D | May play a role in spermatogenesis | 2.3 | 0.01 |
| Q9HBM0 | Plays a pivotal role in the establisment of adherens | 2.3 | 0.06 |
| | junctions and their maintenance in adult life | | |
| NOV | Immediate-early protein likely to play a role in cell | 2.3 | 0.04 |
| | growth regulation | | |
| KIAA1533 | GRAM domain | 2.3 | 0.00 |
| VPREB1 | Associates with the Ig-mu chain to form a molecular | 2.3 | 0.00 |
| | complex that is expressed on the surface of pre-B-cells | | |
| | and presumably regulates Ig gene rearrangements in | | |
| | early B-cell differentiation | | |
| Q96NJ4 | signal peptide | 2.3 | 0.02 |
| FCGR2B | Low affinity receptor for the Fc region of complexed | 2.3 | 0.03 |
| | immunoglobulins gamma. receptor. Involved in various | | |
| | effector and regulatory functions such as phagocytosis of | | |
| | immune complexes and modulation of antibody | | |
| | production by B-cells; | | |
| ENSG00000110900 | CD9/CD37/CD63 antigen | 2.3 | 0.00 |
| Q9HCK1 | low complexity | 2.3 | 0.06 |
| BCDO2 | Asymmetrically cleaves beta-carotene at the double | 2.3 | 0.04 |
| | bond resulting in the formation of beta-carotenal and | | |
| | beta-ionone. Lycopene is also oxidatively cleaved. | | |
| HOXB2 | Sequence-specific transcription factor which is part of a | 2.3 | 0.03 |
| | developmental regulatory system that provides cells with | | |
| | specific positional identities on the anterior-posterior axis | | |
| TUSC4 | Bipartite nuclear localization signal | 2.3 | 0.03 |
| Q8NBF0 | low complexity; signal peptide; transmembrane | 2.3 | 0.03 |
| ABL2 | Tyrosine-protein kinase ABL2; Tyrosine kinase ARG | 2.3 | 0.02 |
| Q8TF23 | BED finger; Cytochrome c heme-binding site; KRAB | 2.3 | 0.02 |
| | box; Zn-finger, C2H2 subtype | | |
| Q8IUC6 | Proline-rich extensin | 2.3 | 0.03 |
| CD151 | Essential for the proper assembly of the glomerular and | 2.3 | 0.00 |
| | tubular basement membranes in kidney | | |
| IER3 | Immediate early response 3 protein; Radiation-inducible | 2.3 | 0.03 |
| | immediate-early gene IEX-1; GLY96; PACAP- | | |
| | responsive Differentiation-dependent gene 2 protein; | | |
| | DIF-2 protein | | |
| Q9NXL6 | SID1 transmembrane family, member 1 | 2.3 | 0.06 |
| C14orf79 | unknown | 2.3 | 0.03 |
| C16orf44 | BTB/POZ domain; Kelch repeat | 2.3 | 0.02 |
| KIAA1404 | Protein KIAA1404 | 2.3 | 0.00 |
| ENSG00000106603 | signal peptide; transmembrane | 2.2 | 0.05 |
| Q86XN7; | Aldehyde dehydrogenase; Proline-rich extensin | 2.2 | 0.02 |
| Q9H9M1 | | | |
| RAB3B | Protein transport. Probably involved in vesicular traffic | 2.2 | 0.04 |
| RHOC | Protein phosphatase 2C-like | 2.2 | 0.04 |
| NDUFB4 | Transfer of electrons from NADH to the respiratory | 2.2 | 0.02 |
| | chain. The immediate electron acceptor is believed to be | | |
| | ubiquinone | | |
| STAMBP | Bipartite nuclear localization signal; Mov34 family | 2.2 | 0.03 |
| ECE2 | Converts big endothelin-1 to endothelin-1 | 2.2 | 0.05 |
| LCP2 | Involved in T cell antigen receptor mediated signaling | 2.2 | 0.04 |
| OASL | Binds double-stranded RNA and DNA, but no OAS | 2.2 | 0.03 |
| | activity | | |
| CEBPD | C/EBP is a DNA-binding protein that recognizes two | 2.2 | 0.02 |
| | different motifs: the CCAAT homology common to | | |
| | many promoters and the enhanced core homology | | |
| | common to many enhancers. Important transcriptional | | |
| | activator in the regulation of genes involved in immune | | |
| | and inflammatory responses, may play an important role | | |
| | in the regulation of the several genes associated with | | |
| | activation and/or differentiation of macrophages | | |
| SOD2 | Destroys radicals which are normally produced within | 2.2 | 0.02 |
| | the cells and which are toxic to biological systems | | |
| Q8N316 | Protein kinase; Serine/Threonine protein kinase | 2.2 | 0.01 |
| KIAA1847 | Zinc finger CCCH-type with G patch domain protein 9 | 2.1 | 0.00 |
| Q9UFQ7 | low complexity; transmembrane | 2.1 | 0.04 |
| ADAMTS6 | ADAMTS-6 precursor; A disintegrin and | 2.1 | 0.02 |
| | metalloproteinase with thrombospondin motifs 6 | | |
| S100A12 | Calcitermin possesses antifungal activity against | 2.1 | 0.04 |
| | C. albicans & is also active vs. E. coli, P. aeruginosa but | | |
| | not Listeria and S. aureus | | |
| DBC1 | Deleted in bladder cancer chromosome region candidate | 2.1 | 0.05 |
| | 1. | | |
| KRTAP2-4 | Keratin, high sulfur B2 protein; von Willebrand factor, | 2.1 | 0.00 |
| | type C | | |
| PEMT | Catalyzes three sequential methylation of PE by AdoMet, | 2.1 | 0.05 |
| | thus producing phosphatidylcholine | | |
| TNIP1 | Interacts with zinc finger protein A20/TNFAIP3 and | 2.1 | 0.05 |
| | inhibits TNF-induced NFκB-dependent gene expression | | |
| | by interfering with an RIP- or TRAF2-mediated | | |
| | transactivation signal. Over-expression can inhibit HIV | | |
| | replication. | | |
| PTP4A3 | Prenyl group binding site (CAAX box); Tyrosine | 2.1 | 0.00 |
| | specific protein phosphatase and dual specificity protein | | |
| | phosphatase | | |
| SOD3 | Destroys radicals which are normally produced within | 2.1 | 0.04 |
| | the cells and which are toxic to biological systems | | |
| ADORA2A | Receptor for adenosine. The activity of this receptor is | 2.1 | 0.05 |
| | mediated by G proteins which activate adenylyl cyclase | | |
| C14orf166 | Protein C14orf166 | 2.1 | 0.06 |
| PELI1 | Scaffold protein involved in the IL-1 signaling pathway | 2.1 | 0.01 |
| | via its interaction with the complex containing IRAK | | |
| | kinases and TRAF6. Required for NFκB activation and | | |
| | IL-8 gene expression in response to IL-1 | | |
| PPP1R15B | protein phosphatase 1, regulatory subunit 15B. | 2.1 | 0.05 |
| KLHL18; | Kelch-like protein 18 | 2.1 | 0.02 |
| CHCHD3 | Protein of unknown function DUF737 | 2.1 | 0.04 |
| O94940 | SAM (and some other nucleotide) binding motif | 2.1 | 0.01 |
| TEX15 | testis expressed sequence 15. | 2.1 | 0.05 |
| DAB1 | Adapter molecule functioning in neural development. | 2.1 | 0.05 |
| | May regulate SIAH1 activity | | |
| RAB5B | Protein transport. Probably involved in vesicular traffic | 2.1 | 0.02 |
| IGHG3 | Ig alpha is the major immunoglobulin class in body | 2.1 | 0.06 |
| | secretions. It may serve both to defend against local | | |
| | infection and to prevent access of foreign antigens to the | | |
| | general immunologic system | | |
| INSIG1 | May play a role in growth and differentiation of tissues | 2.1 | 0.03 |
| | involved in metabolic control. May play a regulatory role | | |
| | during G0/G1 transition of cell growth | | |
| CXCL6 | Chemotactic for neutrophil granulocytes | 2.1 | 0.03 |
| GPR84 | Rhodopsin-like GPCR superfamily | 2.1 | 0.04 |
| Q96G36 | Alpha tubulin; Epsilon tubulin; Tubulin/FtsZ protein | 2.0 | 0.01 |
| Q8NFQ8 | AF464140. | 2.0 | 0.03 |
| Q9BRC8 | low complexity; transmembrane | 2.0 | 0.03 |
| CACNA1H | Voltage-sensitive calcium channels (VSCC) mediate the | 2.0 | 0.00 |
| | entry of calcium ions into excitable cells and are also | | |
| | involved in a variety of calcium-dependent processes, | | |
| | including muscle contraction, hormone or | | |
| | neurotransmitter release, gene expression, cell motility, | | |
| | cell division and cell death. | | |
| ABHD5 | Alpha/beta hydrolase; Esterase/lipase/thioesterase, active | 2.0 | 0.01 |
| | site; Prolyl aminopeptidase S33 | | |
| Q8NBR8 | Immunoglobulin-like; Ig/major histocompatibility | 2.0 | 0.02 |
| | complex | | |
| POLR2D | DNA-dependent RNA polymerase catalyzes the | 2.0 | 0.06 |
| | transcription of DNA into RNA using the four | | |
| | ribonucleoside triphosphates as substrates. Associates | | |
| | with POLR2G | | |
| O76052 | low complexity | 2.0 | 0.05 |
| Q8N7I3 | Immunoglobulin-like | 2.0 | 0.02 |
| Q9Y3B9 | Bipartite nuclear localization signal | 2.0 | 0.06 |
| HIF1A | Functions as a master transcriptional regulator of the | 2.0 | 0.02 |
| | adaptive response to hypoxia. Under hypoxic conditions | | |
| | activates the transcription of over 40 genes, including, | | |
| | erythropoietin, glucose transporters, glycolytic enzymes, | | |
| | vascular endothelial growth factor, and other genes | | |
| | whose protein products increase oxygen delivery or | | |
| | facilitate metabolic adaptation to hypoxia. Plays an | | |
| | essential role in embryonic vascularization, tumor | | |
| | angiogenesis and pathophysiology of ischemic disease. | | |
| Q9BTK5 | G-protein beta WD-40 repeat | 2.0 | 0.04 |
| PHKG1 | Phosphorylase b kinase catalyzes the phosphorylation of | 2.0 | 0.02 |
| | serine in certain substrates, including troponin I | | |
| PLEKHB2 | Pleckstrin-like; Proline-rich extensin | 2.0 | 0.02 |
| CCNB1 | Essential for the control of the cell cycle at the G2/M | 2.0 | 0.00 |
| | (mitosis) transition | | |
| O15069 | Nascent polypeptide-associated complex NAC | 2.0 | 0.05 |
| EIF5A | Precise role of eIF-5A in protein biosynthesis is not | 2.0 | 0.05 |
| | known but it functions by promoting the formation of the | | |
| | first peptide bond | | |
| HDAC10 | Responsible for the deacetylation of lysine residues on | 2.0 | 0.01 |
| | the N-terminal part of the core histones (H2A, H2B, H3 | | |
| | and H4). Histone deacetylation gives a tag for epigenetic | | |
| | repression and plays an important role in transcriptional | | |
| | regulation, cell cycle progression and developmental | | |
| | events. | | |
| Q9H908 | unknown | 2.0 | 0.05 |
| BTBD12 | BTB/POZ domain | 2.0 | 0.05 |
| RBMS3 | Paraneoplastic encephalomyelitis antigen; RNA-binding | 1.9 | 0.04 |
| | region RNP-1 | | |
| FBXL12 | Substrate-recognition component of the SCF (SKP1- | 1.9 | 0.04 |
| | CUL1-F-box protein)-type E3 ubiquitin ligase complex | | |
| SPG7 | 2Fe—2S ferredoxin; Peptidase M41 | 1.9 | 0.01 |
| MGAT5 | Catalyzes the addition of N-acetylglucosamine in beta 1- | 1.9 | 0.02 |
| | 6 linkage to the alpha-linked mannose of biantennary N- | | |
| | linked oligosaccharides. | | |
| SIAT4C | It may catalyze the formation of the NeuAc-alpha-2,3- | 1.9 | 0.01 |
| | Gal-beta-1,3-GalNAc- or NeuAc-alpha-2,3-Gal-beta-1,3- | | |
| | GlcNAc-sequences found in terminal carbohydrate | | |
| | groups of glycoproteins and glycolipids. | | |
| IL12B | Cytokine that can act as a growth factor for activated T | 1.9 | 0.00 |
| | and NK cells, enhances the lytic activity of | | |
| | NK/lymphokine-activated killer cells, stimulates the | | |
| | production of IFN-γ by resting PBMC | | |
| GGN | Proline-rich extensin; Proline-rich region | 1.9 | 0.01 |
| NFKBIA | Inhibits NFκB by complexing with and trapping it in the | 1.9 | 0.00 |
| | cytoplasm. May be involved in regulation of | | |
| | transcriptional responses to NF-kappa-B, including cell | | |
| | adhesion, immune and proinflammatory responses, | | |
| | apoptosis, differentiation and growth. | | |
| ENSG00000153820 | low complexity | 1.9 | 0.01 |
| CYP19A1 | Catalyzes the formation of aromatic C18 estrogens from | 1.9 | 0.01 |
| | C19 androgens | | |
| MUC13 | EGF-like domain; SEA domain | 1.9 | 0.02 |
| CGI-117 | Protein CGI-117 | 1.9 | 0.00 |
| HRMT1L6 | Mono and asymmetric dimethylation of guanidino | 1.9 | 0.02 |
| | nitrogens of arginyl residues present in a glycine and | | |
| | arginine-rich domain | | |
| WTAP | Wilms-tumor 1-associating protein; Putative pre-mRNA | 1.9 | 0.05 |
| | splicing regulator female-lethal(2D) homolog | | |
| DPYS | Dihydropyrimidinase; DHPase; Hydantoinase; DHP | 1.9 | 0.05 |
| LILRB2 | Receptor for class I MHC antigens. Recognizes a broad | 1.9 | 0.06 |
| | spectrum of HLA-A, HLA-B, HLA-C and HLA-G | | |
| | alleles. Involved in the down-regulation of the immune | | |
| | response and the development of tolerance. | | |
| HIAT1 | General substrate transporter; Sugar transporter | 1.9 | 0.04 |
| | superfamily; Tetracycline resistance protein | | |
| KDELR1 | Required for the retention of luminal endoplasmic | 1.9 | 0.04 |
| | reticulum proteins. Required for normal vesicular traffic | | |
| | through Golgi. | | |
| GPC5 | Cell surface proteoglycan that bears heparan sulfate | 1.9 | 0.05 |
| ATP4A | Catalyzes the hydrolysis of ATP coupled with the | 1.9 | 0.05 |
| | exchange of H(+) and K(+) ions across the plasma | | |
| | membrane. Responsible for acid production in the | | |
| | stomach | | |
| C5orf13 | Neuronal protein 3.1; p311 protein | 1.9 | 0.05 |
| KLK14 | Kallikrein-14 precursor; Kallikrein-like protein 6; KLK- | 1.9 | 0.00 |
| | L6 | | |
| Q9NZY8 | unknown | 1.9 | 0.02 |
| ZNF80 | May be involved in transcriptional regulation | 1.9 | 0.04 |
| SMARCA2 | Transcriptional coactivator cooperating with nuclear | 1.9 | 0.02 |
| | hormone receptors to potentiate transcriptional activation | | |
| MXD3 | Basic helix-loop-helix dimerization domain bHLH | 1.9 | 0.03 |
| PDCD6 | Calcium-binding protein required for T cell receptor-, | 1.9 | 0.02 |
| | Fas-, and glucocorticoid-induced cell death. May mediate | | |
| | Ca(2+)-regulated signals along the death pathway | | |
| CYorf14 | Hypothetical protein CYorf14 | 1.9 | 0.02 |
| C20orf97 | Disrupts insulin signaling by binding directly to Akt | 1.9 | 0.05 |
| | kinases and blocking their activation. Interacts with | | |
| | MAPK kinases and regulates activation of MAP kinases. | | |
| Q9HCM3 | Bacterial regulatory protein, LuxR family | 1.9 | 0.03 |
| PFKFB3 | Synthesis and degradation of fructose 2,6-bisphosphate | 1.9 | 0.02 |
| NID2 | Cell adhesion glycoprotein which is widely distributed in | 1.9 | 0.05 |
| | base-ment membranes. Binds to collagens I & IV, | | |
| | perlecan, laminin 1. Probably has role in cell- | | |
| | extracellular matrix interactions | | |
| Q8IZ83 | Aldehyde dehydrogenase | 1.9 | 0.03 |
| Q9Y3H6 | Bipartite nuclear localization signal | 1.9 | 0.04 |
| FCN3 | Involved in the serum exerting lectin activity. | 1.9 | 0.01 |
| Q9NVS3 | IQ calmodulin-binding region | 1.8 | 0.02 |
| SEH1L | Nucleoporin SEH1-like; SEC13-like protein | 1.8 | 0.01 |
| C6orf115 | Protein C6orf115 | 1.8 | 0.04 |
| LRP16 | Protein LRP16 | 1.8 | 0.02 |
| TFRC | Transferrin receptor is necessary for development of | 1.8 | 0.05 |
| | erythrocytes and the nervous system. Cellular uptake of | | |
| | iron occurs via receptor-mediated endocytosis of ligand- | | |
| | occupied transferrin receptor into specialized endosomes. | | |
| MAP1LC3A | Probably involved in formation of autophagosomal | 1.8 | 0.05 |
| | vacuoles | | |
| Q7Z4E7 | low complexity | 1.8 | 0.01 |
| O60739 | Probably involved in translation | 1.8 | 0.00 |
| LGR7 | Receptor for relaxins. The activity of this receptor is | 1.8 | 0.05 |
| | mediated by G proteins leading to stimulation of | | |
| | adenylate cyclase and an increase of cAMP. | | |
| CFHL5 | Involved in complement regulation | 1.8 | 0.01 |
| Q8N9G6 | low complexity | 1.8 | 0.05 |
| ICAM1 | ICAM proteins are ligands for the leukocyte adhesion | 1.8 | 0.01 |
| | LFA-1 protein (Integrin alpha-L/beta-2) | | |
| Q9H606 | Proline-rich region | 1.8 | 0.06 |
| ENSG0000154511 | low complexity; transmembrane | 1.8 | 0.06 |
| SLC24A2 | Critical component of the visual transduction cascade. | 1.8 | 0.03 |
| Q9H8S7 | Bipartite nuclear localization signal | 1.8 | 0.00 |
| TRAPPC4 | May play a role in vesicular transport from endoplasmic | 1.8 | 0.03 |
| | reticulum to Golgi | | |
| ZNF513 | Zn-finger, C2H2 type | 1.8 | 0.03 |
| CLK3 | Phosphorylates seine- and arginine-rich (SR) proteins of | 1.8 | 0.01 |
| | the spliceosomal complex may be a constituent of a | | |
| | network of regulatory mechanisms that enable SR | | |
| | proteins to control RNA splicing. Phosphorylates serines, | | |
| | threonines and tyrosines | | |
| Q9NTF2 | Prenyl group binding site (CAAX box) | 1.8 | 0.04 |
| SIGLEC10 | Putative adhesion molecule that mediates sialic-acid | 1.8 | 0.02 |
| | dependent binding to cells. | | |
| SLC22A11 | General substrate transporter | 1.8 | 0.03 |
| AQP9 | Forms a channel with a broad specificity. Mediates | 1.8 | 0.02 |
| | passage of a wide variety of non-charged solutes. | | |
| SSBP3 | May be involved in transcription regulation of the alpha | 1.8 | 0.05 |
| | 2(I) collagen gene where it binds to the single-stranded | | |
| | polypyrimidine sequences in the promoter region | | |
| MAPK11 | Kinase involved in a signal transduction pathway that is | 1.8 | 0.05 |
| | activated by changes in the osmolarity of the | | |
| | extracellular environment, by cytokines, or by | | |
| | environmental stress. Phosphorylates ATF2 | | |
| MAPK6 | Phosphorylates microtubule-associated protein 2 | 1.8 | 0.02 |
| | (MAP2). May promote entry in the cell cycle | | |
| GHR | Receptor for pituitary gland growth hormone involved in | 1.8 | 0.03 |
| | regulating postnatal body growth. On ligand binding, | | |
| | couples to the JAK2/STAT5 pathway | | |
| TREX1 | Exonuclease; Proline-rich region | 1.8 | 0.03 |
| CBARA1 | Bipartite nuclear localization signal; Calcium-binding | 1.8 | 0.01 |
| | EF-hand | | |
| Q96RH9 | MUSP1. | 1.8 | 0.05 |
| MTHFD2 | Bifunctional NAD-dependent methylenetetrahydrofolate | 1.8 | 0.04 |
| | dehydrogenase/cyclohydrolase, mitochondrial precursor | | |
| PLXNA4 | Cell surface receptor IPT/TIG; Plexin | 1.8 | 0.01 |
| KIAA0084 | Hypothetical protein KIAA0084; HA2022 | 1.8 | 0.04 |
| RPS9 | 40S ribosomal protein S9 | 1.8 | 0.02 |
| PSMD1 | Acts as a regulatory subunit of the 26 proteasome which | 1.8 | 0.02 |
| | is involved in ATP-dependent degradation of | | |
| | ubiquitinated proteins | | |
| Q9Y6U7 | Proline-rich region; Zn-finger, RING | 1.8 | 0.03 |
| RIMBP2 | Plays role in the synaptic transmission as bifunctional | 1.8 | 0.02 |
| | linker. | | |
| PAP | Might be a stress protein involved in the control of | 1.8 | 0.05 |
| | bacterial proliferation | | |
| ZNF274 | May function as a transcriptional repressor | 1.7 | 0.04 |
| ZIM2 | May function as a transcription factor | 1.7 | 0.05 |
| TFPI | Inhibits factor Xa directly and, in a Xa-dependent way, | 1.7 | 0.01 |
| | inhibits VIIa/tissue factor activity, presumably by | | |
| | forming a quaternary Xa/LACI/VIIa/TF complex. It | | |
| | possesses an antithrombotic action and also the ability to | | |
| | associate with lipoproteins in plasma | | |
| ALOX15 | Converts arachidonic acid to 15S- | 1.7 | 0.04 |
| | hydroperoxyeicosatetraenoic acid. Acts on C-12 of | | |
| | arachidonate as well as on linoleic acid | | |
| LAIR1 | Immunoglobulin-like | 1.7 | 0.04 |
| Q8N3D0 | low complexity | 1.7 | 0.03 |
| HYAL2 | Hyaluronidase that hydrolyzes high molecular weight | 1.7 | 0.03 |
| | hyaluronic acid to produce an intermediate-sized | | |
| | product. | | |
| EHD1 | Acts in early endocytic membrane fusion and membrane | 1.7 | 0.01 |
| | trafficking of recycling endosomes | | |
| C1orf22 | Putative aipha-mannosidase C1orf22 | 1.7 | 0.01 |
| GPR52 | Orphan receptor | 1.7 | 0.03 |
| Q96CX6 | Leucine-rich repeat | 1.7 | 0.01 |
| Q9BYX4 | CARD interaction domain; DEAD/DEAH box helicase | 1.7 | 0.04 |
| SHOX2 | May be growth regulator and have a role in specifying | 1.7 | 0.05 |
| | neural systems involved in processing somatosensory | | |
| | information. | | |
| PPP2R1A | The PR65 subunit of protein phosphatase 2A serves as a | 1.7 | 0.00 |
| | scaffolding molecule to coordinate the assembly of the | | |
| | catalytic subunit and a variable regulatory B subunit | | |
| ATR | Phosphatidylinositol 3- and 4-kinase, FAT; FATC; | 1.7 | 0.04 |
| KIAA2010 | EVH1; Protein of unknown function DUF625 | 1.7 | 0.01 |
| MPI | Involved in the synthesis of the GDP-mannose and | 1.7 | 0.03 |
| | dolichol-phosphate-mannose required for critical | | |
| | mannosyl transfers | | |
| Q9H9X6 | Bipartite nuclear localization signal | 1.7 | 0.03 |
| SPIB | Sequence specific transcriptional activator which binds | 1.7 | 0.03 |
| | to the PU-box, a purine-rich DNA sequence and can act | | |
| | as a lymphoid-specific enhancer. Promotes development | | |
| | of plasmacytoid dendritic cells (pDCs), also known as | | |
| | type 2 DC precursors (pre-DC2) or natural interferon | | |
| | (IFN)-producing cells. These cells have the capacity to | | |
| | produce large amounts of interferon and block viral | | |
| | replication. | | |
| Q9BW08 | Bipartite nuclear localization signal; PWWP domain | 1.7 | 0.05 |
| EPS15 | Involved in cell growth regulation. May be involved in | 1.7 | 0.01 |
| | the regulation of mitogenic signals and control of cell | | |
| | proliferation. Involved in the internalization of ligand- | | |
| | inducible receptors of the receptor tyrosine kinase (RTK) | | |
| | type, in particular EGFR | | |
| DPH5 | Required for the methylation step in diphthamide | 1.7 | 0.04 |
| | biosynthesis | | |
| REL | Proto-oncogene that may play a role in differentiation | 1.7 | 0.01 |
| | and lymphopoiesis. May function as a transcriptional | | |
| | transactivator | | |
| SIAT8A | Involved in the production of GD3 and GT3 from GM3 | 1.7 | 0.02 |
| ADAMTS13 | Neutral zinc metalloprotease ADAM/reprolysin M12B | 1.7 | 0.04 |
| LY86 | May cooperate with CD180 and TLR4 to mediate the | 1.7 | 0.01 |
| | innate immune response to bacterial LPS and cytokine | | |
| | production. Important for efficient CD180 cell surface | | |
| | expression | | |
| FBLN2 | Its binding to fibronectin and some other ligands is Ca | 1.7 | 0.03 |
| | dependent | | |
| ADPRHL1 | ADP-ribosylglycohydrolase | 1.7 | 0.01 |
| TRPV6 | Ankyrin; Ion transport protein | 1.7 | 0.04 |
| TSC22D1 | Transcriptional repressor. Acts on the C-type natriuretic | 1.7 | 0.01 |
| | peptide (CNP) promoter | | |
| SAS | Sarcoma amplified sequence; Tetraspanin-31; Tspan-31 | 1.7 | 0.01 |
| ASCL1 | Activates transcription by binding to the E box and may | 1.7 | 0.02 |
| | play a role at early stages of development of specific | | |
| | neural lineages. | | |
| Q9BVM2 | DPCD protein. | 1.7 | 0.01 |
| PRPF8 | Bipartite nuclear localization signal; Mov34 family | 1.7 | 0.06 |
| KPNA4 | Functions in nuclear protein import as an adapter protein | 1.7 | 0.01 |
| | for nuclear receptor KPNB1. Binds specifically and | | |
| | directly to substrates containing either a simple or | | |
| | bipartite NLS motif. | | |
| GLB1L | Glycoside hydrolase, family 35 | 1.7 | 0.04 |
| CCNB1IP1 | E3 ubiquitin ligase. Modulates cyclin B levels and | 1.7 | 0.01 |
| | participates in the regulation of cell cycle progression | | |
| | through the G2 phase. | | |
| CD44 | Receptor for hyaluronic acid (HA). Mediates cell-cell | 1.7 | 0.00 |
| | and cell-matrix interactions through its affinity for HA. | | |
| | Also involved in lymphocyte activation, recirculation | | |
| | and homing, and in hematopoiesis. | | |
| Q8NEQ3 | unknown | 1.7 | 0.03 |
| Q8N2I6; | BTB/POZ domain; G-protein beta WD-40 repeat; K+ | 1.7 | 0.03 |
| Q8TBC3 | channel tetramerisation | | |
| SNAPC2 | Part of the SNAPc complex required for the transcription | 1.7 | 0.06 |
| | of both RNA polymerase II and III small-nuclear RNA | | |
| | genes. | | |
| ARFRP1 | Possibly involved in plasma membrane-related signaling | 1.7 | 0.02 |
| | events | | |
| ARTN | Proline-rich region; Transforming growth factor beta | 1.7 | 0.04 |
| | (TGFb) | | |
| LSM10 | Binds specifically to U7 snRNA | 1.7 | 0.00 |
| O60844 | Jacalin-related lectin | 1.7 | 0.05 |
| PDCD4 | Initiation factor eIF-4 gamma, MA3 | 1.7 | 0.05 |
| RIMS2 | Rab effector involved in exocytosis. May act as scaffold | 1.7 | 0.02 |
| | protein | | |
| Q86W66 | esophageal cancer associated protein. | 1.7 | 0.01 |
| AP2S1 | Component of the adaptor complexes which link clathrin | 1.7 | 0.05 |
| | to receptors in coated vesicles. | | |
| Q8WUB2 | protein predicted by clone 23733. | 1.7 | 0.03 |
| GCH1 | Isoform GCH-1 is the functional enzyme, the potential | 1.7 | 0.02 |
| | function of the euzymatically inactive isoforms remains | | |
| | unknown | | |
| Q9UPX5 | ATP/GTP-binding site motif A (P-loop) | 1.7 | 0.06 |
| NUDT4 | NUDIX hydrolase | 1.7 | 0.06 |
| HSPB1 | Involved in stress resistance and actin organization | 1.7 | 0.02 |
| Q9H679 | low complexity; signal peptide; transmembrane | 1.7 | 0.05 |
| ENSG0000087116 | Immunoglobulin-like | 1.7 | 0.01 |
| MAN2B1 | Necessary for the catabolism of N-linked carbohydrates | 1.7 | 0.02 |
| | released during glycoprotein turnover. | | |
| GK | Key enzyme in the regulation of glycerol uptake and | 1.7 | 0.02 |
| | metabolism | | |
| NUTF2 | Facilitates protein transport into the nucleus. Interacts | 1.7 | 0.01 |
| | with the nucleoporin p62 and with Ran. | | |
| Q9H8H0 | coiled-coil; low complexity | 1.7 | 0.05 |
| GALNT9 | Ricin B lectin domain | 1.7 | 0.02 |
| F13A1 | Factor XIII is activated by thrombin and calcium ion to a | 1.6 | 0.05 |
| | transglutaminase that catalyzes the formation of cross- | | |
| | links between fibrin chains, thus stabilizing the fibrin | | |
| | clot. | | |
| Q96AP0 | low complexity | 1.6 | 0.05 |
| RPS9 | 40S ribosomal protein S9 | 1.6 | 0.02 |
| SLC2A14 | Facilitative glucose transporter. Probably a neuronal | 1.6 | 0.01 |
| | glucose transporter | | |
| ATP5F1 | ATP synthase B chain, mitochondrial precursor | 1.6 | 0.04 |
| DDX21 | Can unwind double-stranded RNA (helicase) and can | 1.6 | 0.01 |
| | fold or introduce a secondary structure to a single- | | |
| | stranded RNA (foldase). Functions as cofactor for c-Jun- | | |
| | activated transcription. | | |
| TTLL3 | Tubulin tyrosine ligase-like protein 3; HOTTL | 1.6 | 0.06 |
| SMPDL3A | Acid sphingomyelinase-like phosphodiesterase 3a | 1.6 | 0.03 |
| | precursor; | | |
| CPN2 | May play important roles in selective fasciculation and | 1.6 | 0.01 |
| | zone-to-zone projection of the primary olfactory axons | | |
| AURKAIP1; | May act as a negative regulator of Aurora-A kinase, by | 1.6 | 0.02 |
| AIP; AKIP | down-regulation through proteasome-dependent | | |
| | degradation | | |
| Q9P1V9 | low complexity | 1.6 | 0.02 |
| Q9NZE3 | KH domain, type 1; Zn-finger, RING | 1.6 | 0.03 |
| SLC29A1 | Delayed-early response protein/equilibrative nucleoside | 1.6 | 0.04 |
| | transporter | | |
| C1orf24 | Niban protein | 1.6 | 0.02 |
| Q9H3U1 | Armadillo repeat; TPR repeat | 1.6 | 0.01 |
| ARF4 | Involved in protein trafficking; may modulate vesicle | 1.6 | 0.03 |
| | budding and uncoating within the Golgi apparatus | | |
| ZNF185 | May be involved in the regulation of cellular | 1.6 | 0.03 |
| | proliferation and/or differentiation | | |
| NUBP1 | Nucleotide-binding protein 1; NBP 1 | 1.6 | 0.04 |
| GPR25 | Orphan receptor | 1.6 | 0.01 |
| Q9Y2K2 | Protein kinase; Serine/Threonine protein kinase; | 1.6 | 0.01 |
| | Tyrosine protein kinase | | |
| LILRA1 | May act as soluble receptor for class I MHC antigens | 1.6 | 0.05 |
| ENSG00000173961 | Bipartite nuclear localization signal; HMG1/2 (high | 1.6 | 0.05 |
| | mobility group) box; High mobility group proteins | | |
| | HMG1 and HMG2 | | |
| IBRDC2 | Zn-finger, RING; Zn-finger, cysteine-rich C6HC | 1.6 | 0.04 |
| ADIPOR1 | Receptor for globular and full-length adiponectin | 1.6 | 0.01 |
| | (APM1), an essential hormone secreted by adipocytes. | | |
| | Probably involved in metabolic pathways that regulate | | |
| | lipid metabolism such as fatty acid oxidation. | | |
| NDUFS7 | NADH-ubiquinone oxidoreductase 20 kDa subunit, mitochondrial | 1.6 | 0.01 |
| | precursor; Complex I-20KD; CI-20KD; PSST | | |
| | subunit | | |
| FGF6 | Can transform NIH 3T3 cells. Exhibits strong mitogenic | 1.6 | 0.02 |
| | and angiogenic properties | | |
| ABHD5 | Alpha/beta hydrolase; Esterase/lipase/thioesterase, active | 1.6 | 0.04 |
| | site; Prolyl aminopeptidase S33 | | |
| ABCC8 | Mono-heme cytochrome b. Regulator of ATP-sensitive | 1.6 | 0.06 |
| | K+ channels and insulin release | | |
| UFC1 | E2-like enzyme which forms an intermediate with UFM1 | 1.6 | 0.03 |
| | via a thioester linkage | | |
| QPCT | Responsible for the biosynthesis of pyroglutamyl | 1.6 | 0.01 |
| | peptides. | | |
| KIAA0196 | Protein KIAA0196 | 1.6 | 0.04 |
| HMGA1 | HMG-I/Y bind preferentially to the minor groove of A + T | 1.6 | 0.05 |
| | rich regions in double stranded DNA. Also involved in | | |
| | transcription regulation of genes containing, or near to | | |
| | A + T-rich regions | | |
| Q9BV99 | Leucine-rich repeat | 1.6 | 0.00 |
| RAPGEF2 | Guanine nucleotide exchange factor (GEF) for Rap1A | 1.6 | 0.02 |
| | and Rap2B GTPases. It does not interact with cAMP or | | |
| | cGMP | | |
| Q9BRP1 | Heat shock protein DnaJ, N-terminal; Programmed cell | 1.6 | 0.05 |
| | death protein 2, C-terminal | | |
| SPATA5L1 | AAA ATPase; ATP/GTP-binding site motif A (P-loop) | 1.6 | 0.06 |
| BDNF | Promotes the survival of neuronal populations that are all | 1.6 | 0.04 |
| | located either in the central nervous system or directly | | |
| | connected to it. | | |
| C20orf85 | Protein C20orf85 | 1.6 | 0.04 |
| THBD | Thrombomodulin is a specific endothelial cell receptor | 1.6 | 0.04 |
| | that forms a 1:1 stoichiometric complex with thrombin. | | |
| | This complex is responsible for the conversion of protein | | |
| | C to the activated protein C (protein Ca). | | |
| HSD3B1 | 3beta-HSD is a bifunctional enzyme, that catalyzes the | 1.6 | 0.04 |
| | oxidative conversion of hormonal steroids and | | |
| | ketosteroids. | | |
| Q9BYH8 | Ankyrin | 1.6 | 0.00 |
| Q8WUE8 | Protein CGI-96 (PNAS-4). | 1.6 | 0.03 |
| ACR | Acrosin is the major protease of mammalian | 1.6 | 0.03 |
| | spermatozoa. | | |
| Q8N7N1 | unknown | 1.6 | 0.01 |
| PRG1 | May neutralize hydrolytic enzymes | 1.6 | 0.02 |
| ZNF197 | ATP/GTP-binding site motif A (P-loop); Zn-finger, | 1.6 | 0.02 |
| | C2H2 type | | |
| SERPINB1 | Regulates the activity of the neutrophil proteases | 1.6 | 0.03 |
| | elastase, cathepsin G and proteinase-3 | | |
| EPHB2 | Receptor for members of the ephrin-B family | 1.6 | 0.01 |
| AKT2 | General protein kinase capable of phosphorylating | 1.6 | 0.05 |
| | several known proteins | | |
| ADM | AM and PAMP are potent hypotensive and vasodilatator | 1.6 | 0.06 |
| | agents. | | |
| SUI1 | Necessary for scanning and involved in initiation site | 1.6 | 0.01 |
| | selection. Probably involved in translation | | |
| PPM1D | Required for the relief of p53-dependent checkpoint | 1.6 | 0.05 |
| | mediated cell cycle arrest. | | |
| ACTR1A | Component of a multi-subunit complex involved in | 1.6 | 0.00 |
| | microtubule based vesicle motility. It is associated with | | |
| | the centrosome | | |
| Q96MB3 | Protein kinase | 1.6 | 0.02 |
| IFITM1 | Implicated in the control of cell growth. Involved in the | 1.6 | 0.02 |
| | transduction of antiproliferative and homotypic adhesion | | |
| | signals | | |
| ZDHHC4 | Probable palmitoyltransferase ZDHHC4; | 1.6 | 0.03 |
| Q9H8N7 | Zn-finger, C2H2 type | 1.6 | 0.06 |
| EPN3 | Epsin-3; EPS-15 interacting protein 3 | 1.6 | 0.05 |
| DNAJB6 | DnaJ homolog subfamily B member 6; Heat shock | 1.6 | 0.02 |
| | protein J2 | | |
| Q96AG0 | Maternal tudor protein; Staphylococcus nuclease (SNase- | 1.6 | 0.01 |
| | like) | | |
| RFX1 | Regulatory factor essential for MHC class II genes | 1.6 | 0.06 |
| | expression. Binds to the X boxes of MHC class II genes. | | |
| GTF3C5 | Zn-finger, C2H2 type | 1.6 | 0.00 |
| RIN3 | Potential Ras effector protein. May function as a GEF by | 1.6 | 0.05 |
| | exchanging bound GDP for free GTP | | |
| RGS3 | Down-regulates G-protein-mediated release of inositol | 1.6 | 0.04 |
| | phosphates and activation of MAP kinases. | | |
| EBI3 | Cytokine receptor, common beta/gamma chain; | 1.6 | 0.02 |
| | Fibronectin, type III; Long hematopoietin receptor, | | |
| | soluble alpha chain | | |
| FGF20 | Neurotrophic factor that regulates central nervous | 1.6 | 0.01 |
| | development and function | | |
| ZNHIT1 | Bipartite nuclear localization signal; HIT Zn-finger | 1.5 | 0.05 |
| COL7A1 | Stratified squamous epithelial basement membrane | 1.5 | 0.05 |
| | protein that form anchoring fibrils which may contribute | | |
| | to epithelial basement membrane organization and | | |
| | adherence by interacting with extracellular matrix (ECM) | | |
| | proteins e.g type IV collagen | | |
| HERC2 | Cytochrome b5; Regulator of chromosome condensation, | 1.5 | 0.03 |
| | RCC1. | | |
| Q8IV48 | DNA-binding SAP; Exonuclease | 1.5 | 0.06 |
| SELT; Selt | Selenoprotein T precursor | 1.5 | 0.01 |
| CCT2 | Molecular chaperone; assist the folding of proteins upon | 1.5 | 0.05 |
| | ATP hydrolysis. Plays role, in vitro, in the folding of | | |
| | actin and tubulin | | |
| ADAMTS20 | May play a role in tissue-remodeling process occurring | 1.5 | 0.03 |
| | in both normal and pathological conditions | | |
| Q86X29 | Short-chain dehydrogenase/reductase SDR; | 1.5 | 0.03 |
| | TNFR/CD27/30/40/95 cysteine-rich region | | |
| PRSS12 | Plays a role in neuronal plasticity and the proteolytic | 1.5 | 0.02 |
| | action may → structural reorganizations associated with | | |
| | learning & memory | | |
| COPE | The coatomer is a cytosolic protein complex that binds to | 1.5 | 0.02 |
| | dilysine motifs and reversibly associates with Golgi non- | | |
| | clathrin-coated vesicles, which further mediate | | |
| | biosynthetic protein transport from the ER, via the Golgi | | |
| | to the trans Golgi network. | | |
| KIAA1036 | NM_014909 | 1.5 | 0.02 |
| CPNE6 | May function in membrane trafficking. Exhibits calcium- | 1.5 | 0.05 |
| | dependent phospholipid binding properties. | | |
| ALS2CR3 | Amyotrophic lateral sclerosis 2 chromosomal region | 1.5 | 0.04 |
| | candidate gene protein 3 | | |
| MPHOSPH10 | Component of the 60-80S U3 small nucleolar | 1.5 | 0.05 |
| | ribonucleoprotein (U3 snoRNP). Required for the early | | |
| | cleavages during pre-18S ribosomal RNA processing | | |
| CAB39L | Calcium-binding protein 39-like; Mo25-like protein | 1.5 | 0.02 |
| PAPOLB | Polymerase that creates the poly(A) tail of mRNA. | 1.5 | 0.01 |
| THPO | Lineage-specific cytokine affecting the proliferation and | 1.5 | 0.04 |
| | maturation of megakaryocytes from committed | | |
| | progenitor cells. May be major physiological regulator of | | |
| | circulating platelets | | |
| C7orf16 | Inhibits protein phosphatase-2A and protein | 1.5 | 0.05 |
| | phosphatase-1 | | |
| CAPZB | F-actin capping proteins bind in a Ca(2+)-independent | 1.5 | 0.03 |
| | manner to the fast growing ends of actin filaments | | |
| | (barbed end) thereby blocking the exchange of subunits | | |
| | at these ends. | | |
| ZNF124 | ATP/GTP-binding site motif A (P-loop); KRAB box; | 1.5 | 0.03 |
| | Zn-finger, C2H2 type | | |
| RNU3IP2 | Component of a nucleolar small nuclear | 1.5 | 0.01 |
| | ribonucleoprotein particle (snoRNP) thought to | | |
| | participate in the processing and modification of pre- | | |
| | ribosomal RNA | | |
| ANAPC5 | Component of the anaphase promoting | 1.5 | 0.04 |
| | complex/cyclosome (APC/C), a cell cycle-regulated | | |
| | ubiquitin ligase that controls progression through mitosis | | |
| | and the G1 phase of the cell cycle | | |
| ENSG00000134490 | low complexity; signal peptide; transmembrane | 1.5 | 0.00 |
| PPP1R3C | Putative phosphatase regulatory subunit | 1.5 | 0.01 |
| ZFP28 | KRAB box; Zn-finger, C2H2 subtype | 1.5 | 0.03 |
| ADCYAP1 | Stimulates adenylate cyclase in pituitary cells | 1.5 | 0.00 |
| FKBP8 | Has no PPIase/rotamase activity; Regulates myosin | 1.5 | 0.05 |
| | phosphatase activity. Augments Ca²⁺sensitivity of the | | |
| | contractile apparatus | | |
| MTM1 | Dual-specificity phosphatase that acts on both | 1.5 | 0.06 |
| | phosphotyrosine and phosphoserine. Could be involved | | |
| | in a signal transduction pathway necessary for late | | |
| | myogenesis, although its ubiquitous expression suggests | | |
| | a wider function | | |
| Q7Z5U6 | G-protein beta WD-40 repeat | 1.5 | 0.02 |
| DKK1 | Inhibitor of Wnt signaling pathway | 1.5 | 0.02 |
| GALNT11 | Glycosyl transferase, family 2; Ricin B lectin domain | 1.5 | 0.03 |
| IL10 | Inhibits the synthesis of a number of cytokines, including | 1.5 | 0.03 |
| | IFN-gamma, IL-2, IL-3, TNF and GM-CSF produced by | | |
| | activated macrophages and by helper T cells | | |
| TGFBR1 | Receptor for TGF-beta. On ligand binding forms a | 1.5 | 0.01 |
| | receptor complex consisting of two type II and two type I | | |
| | transmembrane serine/threonine kinases, leads to | | |
| | activation of SMAD TFs. | | |
| DDX3X | ATP-dependent RNA helicase. | 1.5 | 0.01 |
| MRPL2 | Ribosomal protein L2 | 1.5 | 0.03 |
| BTRC | Substrate-recognition component of the SCF (SKP1- | 1.5 | 0.01 |
| | CUL1-F-box protein) ubiquitin ligase complex, which | | |
| | mediates the ubiquitination of proteins involved in cell | | |
| | cycle progression, signal transduction and transcription. | | |
| | Regulates the stability of CTNNB1 and participates in | | |
| | Wnt signaling | | |
| FGF10 | May be a growth factor active in the process of wound | 1.5 | 0.02 |
| | healing. Acts as a mitogen in the lung, similar to FGF-7 | | |
| PDCD11 | Involved in the biogenesis of rRNA | 1.5 | 0.01 |
| O75250 | Cytochrome c heme-binding site | 1.5 | 0.05 |
| Wdr68 | WD-repeat protein 68; WD-repeat protein An11 | 1.5 | 0.03 |
| | homolog | | |
| C12orf14 | HEJ1. | 1.5 | 0.04 |
| ATP5E | This is the smallest of the 5 chains of the enzymatic | 1.5 | 0.02 |
| | component (coupling factor CF(1)) of the mitochondrial | | |
| | ATPase complex | | |
| HLA-DMA | Plays a critical role in catalyzing the release of class II | −1.5 | 0.02 |
| | HLA-associated invariant chain-derived peptides (CLIP) | | |
| | from newly synthesized class II HLA molecules and | | |
| | freeing the peptide binding site for acquisition of | | |
| | antigenic peptides | | |
| ENSG00000126970 | Cytochrome c heme-binding site | −1.5 | 0.06 |
| CLECSF6 | C-type lectin; Type II antifreeze protein | −1.5 | 0.04 |
| GIMAP4MS | Component of the 60-80S U3 small nucleolar | −1.5 | 0.01 |
| | ribonucleoprotein (U3 snoRNP). Required for the early | | |
| | cleavages during pre-18S ribosomal RNA processing; | | |
| | Exhibits intrisinic GTPase activity. | | |
| Q86XY4 | Tripin; shugoshin-like 2 | −1.5 | 0.02 |
| Q8IZY6 | Pleckstrin putative G-protein interacting domain; | −1.5 | 0.04 |
| | RhoGAP domain | | |
| INHBE | Inhibins inhibit the secretion of follitropin by the | −1.5 | 0.03 |
| | pituitary gland. | | |
| Q9HBJ8 | collectrin. kidney-specific membrane protein | −1.5 | 0.01 |
| ALDH4A1 | Irreversible conversion of delta-1-pyrroline-5- | −1.5 | 0.05 |
| | carboxylate (P5C), derived either from proline or | | |
| | ornithine, to glutamate. This is a necessary step in the | | |
| | pathway interconnecting the urea and tricarboxylic acid | | |
| | cycles. | | |
| Q9NWR0 | Bipartite nuclear localization signal; Zn-finger, RING | −1.5 | 0.04 |
| VDAC3 | Forms a channel through the mitochondrial outer | −1.5 | 0.02 |
| | membrane that allows diffusion of small hydrophilic | | |
| | molecules | | |
| THRB | High affinity receptor for triiodothyronine | −1.5 | 0.03 |
| PIK3CA | Phosphorylates PtdIns, PtdIns4P and PtdIns(4,5)P2 with | −1.5 | 0.00 |
| | a preference for PtdIns(4,5)P2 | | |
| SOX11 | Probably important in the developing nervous system | −1.5 | 0.01 |
| AKAP4 | Protein kinase A anchoring protein 4, 82 kDa | −1.5 | 0.02 |
| Q9Y6Y3 | IDN3 protein isoform A. | −1.5 | 0.06 |
| O75183 | coiled-coil; low complexity | −1.5 | 0.04 |
| Q9H7R3 | Generic methyltransferase; SAM (and some other | −1.5 | 0.02 |
| | nucleotide) binding motif | | |
| KIAA0084 | Hypothetical protein KIAA0084; HA2022 | −1.5 | 0.03 |
| Q9H7M9 | Immunoglobulin-like | −1.5 | 0.04 |
| SLAMF8 | BCM-like membrane protein precursor. B lymphocyte | −1.5 | 0.01 |
| | activator macrophage expressed | | |
| CORO1B | May be involved in cytokinesis, motility, and signal | −1.5 | 0.03 |
| | transduction | | |
| CALM3 | Calmodulin mediates the control of a large number of | −1.5 | 0.01 |
| | enzymes by Ca(2+). | | |
| MRPS6 | Mitochondrial 28S ribosomal protein S6; S6mt; MRP-S6 | −1.5 | 0.04 |
| FCGR3A | Receptor for the Fc region of IgG. Binds complexed or | −1.5 | 0.03 |
| | aggregated IgG and also monomeric IgG. Mediates | | |
| | antibody-dependent cellular cytotoxicity (ADCC) and | | |
| | other antibody-dependent responses, such as | | |
| | phagocytosis | | |
| POLQ | Could be involved in the repair of interstrand crosslinks | −1.5 | 0.06 |
| ANKRD18A | Ankyrin repeat domain protein 18A | −1.5 | 0.01 |
| NUP54 | Component of the nuclear pore complex, a complex | −1.5 | 0.03 |
| | required for the trafficking across the nuclear membrane | | |
| G6PC | May be a single membrane channel protein acting both | −1.5 | 0.02 |
| | as a hydrolase and a translocase. It is the key enzyme in | | |
| | homeostatic regulation of blood glucose levels | | |
| ZNF646 | May function as a transcription factor | −1.5 | 0.05 |
| ZNF442 | KRAB box; Zn-finger, C2H2 subtype; Zn-finger, C2H2 | −1.5 | 0.04 |
| | type | | |
| UBE2R2 | Ubiquitin-conjugating enzymes | −1.5 | 0.00 |
| NPM3 | May act as a chaperone | −1.5 | 0.02 |
| Q9H6X4 | NULL | −1.5 | 0.03 |
| TFAM | Involved in mitochondrial transcription regulation as an | −1.5 | 0.01 |
| | activator Is able to unwind and bend DNA | | |
| GDNF | Neurotrophic factor that enhances survival and | −1.5 | 0.04 |
| | morphological differentiation of dopaminergic neurons | | |
| | and increases their high-affinity dopamine uptake | | |
| MRPL24 | KOW; Ribosomal protein L24/L26 | −1.5 | 0.01 |
| C10orf45 | Bipartite nuclear localization signal | −1.5 | 0.05 |
| FGD3 | DH domain; Pleckstrin-like; Zn-finger, FYVE type | −1.5 | 0.01 |
| MBNL2 | Zn-finger, C-x8-C-x5-C-x3-H type | −1.5 | 0.04 |
| NUCKS1 | Nuclear ubiquitous casein and cyclin-dependent kinases | −1.5 | 0.06 |
| | substrate; P1 | | |
| TBL1Y | F-box-like protein involved in the recruitment of the | −1.5 | 0.01 |
| | ubiquitin/19S proteasome complex to nuclear receptor- | | |
| | regulated transcription units. | | |
| PCDHB14 | Potential calcium-dependent cell-adhesion protein. May | −1.6 | 0.02 |
| | be involved in the establishment and maintenance of | | |
| | specific neuronal connections in the brain | | |
| PPM1F | Dephosphorylates and concomitantly deactivates CaM- | −1.6 | 0.01 |
| | kinases Promotes apoptosis | | |
| ENSG00000158142 | ATP/GTP-binding site motif A (P-loop); C2 domain | −1.6 | 0.04 |
| Q9H7L1 | low complexity | −1.6 | 0.00 |
| LUC7L | Protein of unknown function DUF259 | −1.6 | 0.03 |
| UPF3B | Bipartite nuclear localization signal; Smg-4/UPF3 | −1.6 | 0.02 |
| DHCR24 | Catalyzes the reduction of the delta-24 double bond of | −1.6 | 0.02 |
| | sterol intermediates. Protects cells from oxidative stress | | |
| | by reducing caspase 3 activity during apoptosis induced | | |
| | by oxidative stress. | | |
| MKRN1 | Makorin-1; RING finger protein 61 | −1.6 | 0.01 |
| TUB | Could be involved in the hypothalamic regulation of | −1.6 | 0.05 |
| | body weight | | |
| ENSG00000176783 | Cytochrome c heme-binding site; RUN domain; Zn- | −1.6 | 0.03 |
| | finger, C-x8-C-x5-C-x3-H type; Zn-finger, FYVE type; | | |
| | Zn-finger, RING | | |
| RTN3 | Reticulon-3; Neuroendocrine-specific protein-like 2; | −1.6 | 0.03 |
| | NSP-like protein II; NSPLII | | |
| Q8N8E1 | Apoptosis-related protein PNAS-1. | −1.6 | 0.05 |
| Q8N283 | Ankyrin | −1.6 | 0.03 |
| Q8IVV7 | low complexity | −1.6 | 0.03 |
| Q7Z6C2 | A-kinase anchoring protein 95 (AKAP95); Bipartite | −1.6 | 0.00 |
| | nuclear localization signal | | |
| Q8N570 | Parathyroid hormone-responsive osteosarcoma B1 | −1.6 | 0.01 |
| | protein | | |
| ALAS1 | 5-aminolevulinate synthase, nonspecific, mitochondrial | −1.6 | 0.05 |
| | precursor; | | |
| MMP20 | Degrades amelogenin, the major protein component of | −1.6 | 0.02 |
| | the enamel matrix and two of the macromolecules | | |
| | characterising the cartilage extracellular matrix | | |
| GALK2 | Acts on GalNAc. Also acts as a galactokinase when | −1.6 | 0.01 |
| | galactose is present at high concentrations | | |
| CENTG3 | GTPase-activating protein for the ADP ribosylation | −1.6 | 0.03 |
| | factor family | | |
| Q9HC06 | CD14 protein. | −1.6 | 0.00 |
| PPM1A | Enzyme with a broad specificity | −1.6 | 0.03 |
| BMP15 | May be involved in follicular development. | −1.6 | 0.01 |
| Q9P107 | Aldehyde dehydrogenase; Protein kinase C, phorbol | −1.6 | 0.05 |
| | ester/diacylglycerol binding; RhoGAP domain | | |
| SPG4 | Probable ATPase involved in the assembly or function of | −1.6 | 0.03 |
| | nuclear protein complexes & maybe in aspects of | | |
| | microtubule dynamics | | |
| NICAL | May be a cytoskeletal regulator that connects NEDD9 to | −1.6 | 0.03 |
| | intermediate filaments | | |
| DEPDC5 | DEP domain containing protein 5 | −1.6 | 0.00 |
| Q7Z5B3 | RIC3 protein. | −1.6 | 0.03 |
| MAGEH1 | Melanoma-associated antigen H1; Restin; Apoptosis- | −1.6 | 0.06 |
| | related protein 1; APR-1 | | |
| VDAC1 | Forms a channel through the mitochondrial outer | −1.6 | 0.05 |
| | membrane and also the plasma membrane; allows | | |
| | diffusion of small hydrophilic molecules. | | |
| THRB | High affinity receptor for triiodothyronine | −1.6 | 0.01 |
| GDNF | Neurotrophic factor that enhances survival and | −1.6 | 0.03 |
| | morphological differentiation of dopaminergic neurons | | |
| ENSG00000188121 | Prenyl group binding site (CAAX box); Proline-rich | −1.6 | 0.01 |
| | extensin; Proline-rich region | | |
| PTGER2 | Receptor for prostaglandin E2 (PGE2). | −1.6 | 0.05 |
| AREG | Bifunctional growth-modulating glycoprotein. | −1.6 | 0.02 |
| HRMT1L3 | Probably methylates the guanidino nitrogens of arginyl | −1.6 | 0.03 |
| | residues in some proteins | | |
| RNF122 | Zn-finger, RING | −1.6 | 0.00 |
| OSGEP | Glycoprotease (M22) metalloprotease | −1.6 | 0.01 |
| Q86VH4 | Leucine-rich repeat | −1.6 | 0.04 |
| LOH12CR1 | LOH1CR12. | −1.6 | 0.00 |
| STAT1 | Signal transducer and activator of transcription that | −1.6 | 0.02 |
| | mediates signaling by interferons (IFNs). | | |
| BTBD7 | BTB/POZ domain | −1.6 | 0.04 |
| TUSC2 | May function as a tumor suppressor, inhibiting colony | −1.6 | 0.01 |
| | formation, causing G1 arrest and ultimately inducing | | |
| | apoptosis | | |
| Q8NF81 | low complexity | −1.6 | 0.01 |
| TGFB1 | Tumour growth factor B1; Multifunctional peptide that | −1.6 | 0.01 |
| | controls proliferation, differentiation, and other | | |
| | functions. | | |
| SERPINB7 | Might function as an inhibitor of Lys-specific proteases. | −1.6 | 0.02 |
| GRM1 | Receptor for glutamate. | −1.6 | 0.02 |
| Q8TBK2 | Nuclear protein SET | −1.6 | 0.02 |
| NT5C1B | 5′-nucleotidase, cytosolic IB; autoimmune infertility- | −1.6 | 0.03 |
| | related protein; | | |
| UTRN | May play a role in anchoring the cytoskeleton to the | −1.6 | 0.05 |
| | plasma membrane (By similarity to dystrophin) | | |
| PRKAG2 | AMPK is responsible for the regulation of fatty acid | −1.6 | 0.03 |
| | synthesis by phosphorylation of acetyl-CoA carboxylase. | | |
| Q9H814 | coiled-coil; low complexity | −1.6 | 0.01 |
| TCF7L2 | Participates in the Wnt signaling pathway and modulates | −1.6 | 0.06 |
| | MYC expression by binding to its promoter in a | | |
| | sequence-specific manner. | | |
| EFNB1 | Binds to the receptor tyrosine kinases EPHB1 and | −1.6 | 0.00 |
| | EPHA1. | | |
| Q96LI9 | Bipartite nuclear localization signal | −1.6 | 0.02 |
| Q8TBP6 | Mitochondrial carrier protein; Mitochondrial substrate | −1.6 | 0.05 |
| | carrier | | |
| GPT | Participates in cellular nitrogen metabolism and in liver | −1.6 | 0.02 |
| | gluconeogenesis | | |
| Q8N1Z9 | Like hepatocellular carcinoma-associated antigen | −1.6 | 0.05 |
| | HCA557b. | | |
| ROBO2 | Fibronectin, type III; Immunoglobulin-like | −1.6 | 0.02 |
| IL13RA1 | Binds IL13 with a low affinity. Together with IL4R- | −1.6 | 0.04 |
| | alpha can form a functional receptor for IL13. Also | | |
| | serves as an alternate accessory protein to the common | | |
| | IL4 receptor gamma chain | | |
| SVIL | ATP/GTP-binding site motif A (P-loop); Bipartite | −1.6 | 0.00 |
| | nuclear localization signal; Gelsolin; Gelsolin region; | | |
| | Villin headpiece | | |
| C6orf80 | Low complexity | −1.6 | 0.02 |
| TMEM1 | May play role in vesicular transport from endoplasmic | −1.6 | 0.03 |
| | reticulum to Golgi | | |
| MYO1B | Motor protein that may participate in process critical to | −1.6 | 0.03 |
| | neuronal development and function such as cell | | |
| | migration, neurite outgrowth and vesicular transport | | |
| SV2B | General substrate transporter; Sugar transporter | −1.6 | 0.02 |
| | superfamily | | |
| VCL | Involved in cell adhesion. May be involved in the | −1.6 | 0.01 |
| | attachment of the actin-based microfilaments to the | | |
| | plasma membrane | | |
| CBX5 | Component of heterochromatin. Recognizes and binds | −1.6 | 0.03 |
| | histone H3 tails methylated at Lys-9, leading to | | |
| | epigenetic repression. | | |
| AKR1B10 | Can efficiently reduce aliphatic and aromatic aldehydes, | −1.6 | 0.03 |
| | and is less active on hexoses. | | |
| MRPL1 | Ribosomal protein L1 | −1.6 | 0.04 |
| Q9BSA9 | NULL | −1.6 | 0.05 |
| ABCD3 | Probable transporter. The nucleotide-binding fold acts as | −1.6 | 0.04 |
| | an ATP-binding subunit with ATPase activity | | |
| FMOD | Affects the rate of fibrils formation. May have a primary | −1.6 | 0.04 |
| | role in collagen fibrillogenesis | | |
| GRIP1 | PDZ/DHR/GLGF domain | −1.6 | 0.03 |
| TMEM22 | Protein of unknown function DUF6 | −1.6 | 0.02 |
| ZFYVE21 | Zn-finger, FYVE type | −1.6 | 0.03 |
| SPPL2A | May act as intramembrane protease | −1.6 | 0.04 |
| CUGBP2 | Paraneoplastic encephalomyelitis antigen; RNA-binding | −1.6 | 0.01 |
| | region RNP-1 | | |
| Q96IW2 | SH2 motif | −1.6 | 0.02 |
| C20orf147 | Haloacid dehalogenase-like hydrolase domain containing | −1.6 | 0.05 |
| | prot. 4 | | |
| Q8N2K3 | low complexity; transmembrane | −1.7 | 0.06 |
| Q9BSD4 | coiled-coil; low complexity | −1.7 | 0.03 |
| LAMC1 | Binding to cells via a high affinity receptor, laminin is | −1.7 | 0.02 |
| | thought to mediate the attachment, migration and | | |
| | organization of cells into tissues during embryonic | | |
| | development by interacting with other extracellular | | |
| | matrix components | | |
| Q8NEH9 | IQ calmodulin-binding region | −1.7 | 0.04 |
| SEMA5B | May act as positive axonal guidance cues | −1.7 | 0.00 |
| TMEM14A | Transmembrane protein 14A | −1.7 | 0.06 |
| ANGPTL1 | Fibrinogen, beta/gamma chain, C-terminal globular | −1.7 | 0.00 |
| Q9NSN6 | TPR repeat | −1.7 | 0.01 |
| PARN | Deadenylation nuclease; poly(A)-specific ribonuclease | −1.7 | 0.03 |
| NPY6R | Neuropeptide Y receptor; Rhodopsin-like GPCR | −1.7 | 0.01 |
| | superfamily | | |
| Q9H089 | ATP/GTP-binding site motif A (P-loop) | −1.7 | 0.05 |
| SKP1A | Essential component of the SCF (SKP1-CUL1-F-box | −1.7 | 0.03 |
| | protein) ubiquitin ligase complex | | |
| CNOT7 | Ubiquitous transcription factor required for a diverse set | −1.7 | 0.02 |
| | of processes. Component of the CCR4 complex. | | |
| MYO1C | Myosins are actin-based motor molecules with ATPase | −1.7 | 0.04 |
| | activity. Unconventional myosins serve in intracellular | | |
| | movement. | | |
| PRKCBP1 | Protein kinase C binding protein 1; Rack7; Cutaneous T- | −1.7 | 0.03 |
| | cell lymphoma associated antigen sel4-3; | | |
| SH3BGRL3 | Could act as a modulator of glutaredoxin biological | −1.7 | 0.01 |
| | activity | | |
| MTHFR | Catalyzes the conversion of 5,10- | −1.7 | 0.01 |
| | methylenetetrahydrofolate to 5-methyltetrahydrofolate, | | |
| | for remethylation to methionine | | |
| NUP155 | Essential component of nuclear pore complex. | −1.7 | 0.04 |
| SMARCA3 | Helicase, C-terminal; SNF2 related domain; Zn-finger, | −1.7 | 0.02 |
| | RING | | |
| SH3MD3 | SH3 domain | −1.7 | 0.03 |
| GSTM1 | Conjugation of reduced glutathione to a wide number of | −1.7 | 0.03 |
| | exogenous and endogenous hydrophobic electrophiles | | |
| C20orf140 | May act as a GTPase activating protein for Rab family | −1.7 | 0.03 |
| | protein(s) | | |
| TAGLN3 | Transgelin-3; Neuronal protein NP25; Neuronal protein | −1.7 | 0.04 |
| | 22; NP22 | | |
| Q96B77 | transmembrane | −1.7 | 0.04 |
| OR52A1 | Putative odorant receptor | −1.7 | 0.03 |
| ACTN1 | F-actin cross-linking protein which is thought to anchor | −1.7 | 0.00 |
| | actin to a variety of intracellular structures. This is a | | |
| | bundling protein | | |
| SLC38A4 | Amino acid/polyamine transporter, family II | −1.7 | 0.05 |
| APP | Functions as a cell surface receptor and can promote | −1.7 | 0.03 |
| | transcription activation through binding to APBB1/Tip60 | | |
| | and inhibit Notch signaling through interaction with | | |
| | Numb. Couples to apoptosis-inducing pathways | | |
| Q96CE7 | FAD-dependent pyridine nucleotide-disulphide | −1.7 | 0.01 |
| | oxidoreductase; Flavin-containing monooxygenase | | |
| | (FMO) 1; | | |
| COQ4 | Ubiquinone biosynthesis protein COQ4 homolog; | −1.7 | 0.05 |
| | Coenzyme Q biosynthesis protein 4 homolog | | |
| FAF1 | Potentiates but cannot initiate FAS-induced apoptosis | −1.7 | 0.03 |
| Q9NTC3 | Eukaryotic/viral aspartic protease, active site | −1.7 | 0.06 |
| ARPC1A | Part of a complex implicated in the control of actin | −1.7 | 0.02 |
| | polymerization in cells | | |
| TRPM4 | Ion transport protein | −1.7 | 0.04 |
| P2RY5 | P2Y purinoceptor 5; P2Y5; Purinergic receptor 5; RB | −1.7 | 0.01 |
| | intron encoded G-protein coupled receptor | | |
| TLK2 | Rapidly and transiently inhibited by phosphorylation | −1.8 | 0.02 |
| | following the generation of DNA double-stranded breaks | | |
| | during S-phase. | | |
| COMMD3 | BUP protein; chromosome 10 open reading frame 8. | −1.8 | 0.01 |
| | COMM domain containing 3 | | |
| ELA3A | Efficient protease with alanine specificity but only little | −1.8 | 0.04 |
| | elastolytic activity | | |
| KIAA0574 | Hypothetical protein KIAA0574 | −1.8 | 0.03 |
| UBE1L | Activates ubiquitin. | −1.8 | 0.03 |
| Q8TB55 | Proline-rich region | −1.8 | 0.05 |
| RAB28 | Ras-related protein Rab-28; Rab-26 | −1.8 | 0.03 |
| ASB8 | Ankyrin repeat and SOCS box protein 8; ASB-8 | −1.8 | 0.01 |
| SNX3 | May be involved in several stages of intracellular | −1.8 | 0.02 |
| | trafficking | | |
| JUND | Binds an AP-1 site and upon cotransfection stimulates | −1.8 | 0.05 |
| | the activity of a promoter that bears an AP-1 site | | |
| Q8NF73 | G-protein beta WD-40 repeat | −1.8 | 0.03 |
| NUP43 | May mediate the assembly of subdomains of the NPC or | −1.8 | 0.04 |
| | facilitate the interaction of transport complexes with the | | |
| | NPC | | |
| DCX | Seems to be required for initial steps of neuronal | −1.8 | 0.00 |
| | dispersion and cortex lamination during cerebral cortex | | |
| | development. | | |
| ASPH | Aspartyl/Asparaginyl beta-hydroxylase, N-terminal | −1.8 | 0.03 |
| RPS6KA5 | Serine/threonine kinase that may play a role in mediating | −1.8 | 0.05 |
| | the growth-factor and stress induced activation of the | | |
| | transcription factor CREB. Essential role in the control | | |
| | of RELA transcriptional activity in response to TNF | | |
| DNTTIP1 | Shown to enhance TdT activity, in vitro | −1.8 | 0.00 |
| ZNF436 | May be involved in transcriptional regulation | −1.8 | 0.02 |
| Q9NX40 | ovarian carcinoma immunoreactive antigen. | −1.8 | 0.04 |
| HAGH | Thiolesterase that catalyzes the hydrolysis of S-D- | −1.8 | 0.01 |
| | lactoyl-glutathione to form glutathione and D-lactic acid | | |
| MGEA6 | Tumor-associated antigen | −1.8 | 0.01 |
| SLC19A1 | Transporter for the intake of folate. | −1.8 | 0.01 |
| SULT4A1 | May catalyze the sulfate conjugation of many drugs, | −1.8 | 0.05 |
| | xenobiotic compounds, hormones, and neurotransmitters. | | |
| NAV1 | ATP/GTP-binding site motif A (P-loop); Bipartite | −1.8 | 0.03 |
| | nuclear localization signal; Inorganic pyrophosphatase | | |
| ANGPTL6 | Fibrinogen, beta/gamma chain, C-terminal globular | −1.8 | 0.03 |
| NDUFA5 | Transfer of electrons from NADH to the respiratory | −1.8 | 0.04 |
| | chain. This is a component of the iron-sulfur (IP) | | |
| | fragment of the enzyme | | |
| C6orf37 | low complexity | −1.8 | 0.05 |
| C9orf86 | ATP/GTP-binding site motif A; Ras GTPase superfamily | −1.8 | 0.04 |
| Q8NAA4 | G-protein beta WD-40 repeat | −1.8 | 0.00 |
| CETN2 | Plays a fundamental role in microtubule-organizing | −1.9 | 0.01 |
| | center structure and function | | |
| CEECAM1 | Endoplasmic reticulum targeting sequence; Glycosyl | −1.9 | 0.03 |
| | transferase, family 25 | | |
| USP6NL | RabGAP/TBC domain | −1.9 | 0.01 |
| FOLH1 | Has both folate hydrolase and N-acetylated-alpha-linked- | −1.9 | 0.01 |
| | acidic dipeptidase (NAALADase) activity. Involved in | | |
| | prostate tumor progression | | |
| CYP51A1 | Catalyzes C14-demethylation of lanosterol. | −1.9 | 0.05 |
| ITGB6 | Integrin alpha-V/beta-6 is a receptor for fibronectin and | −1.9 | 0.06 |
| | cytotactin. It recognizes the sequence R-G-D in its | | |
| | ligands | | |
| RIPK3 | Promotes apoptosis | −1.9 | 0.05 |
| Q8TDG4 | DEAD/DEAH box helicase; Helicase, C-terminal | −1.9 | 0.03 |
| ALS2CR3 | Amyotrophic lateral sclerosis 2 chromosomal region | −1.9 | 0.02 |
| | candidate gene protein 3 | | |
| VMD2L3 | Forms calcium-sensitive chloride channels. May conduct | −1.9 | 0.04 |
| | other physiologically significant anions such as | | |
| | bicarbonate | | |
| O95893 | transmembrane | −1.9 | 0.03 |
| SNTB1 | Adapter protein that binds to and probably organizes the | −1.9 | 0.04 |
| | subcellular localization of a variety of membrane | | |
| | proteins. May link various receptors to the actin | | |
| | cytoskeleton | | |
| MYPN | Endoplasmic reticulum targeting sequence; | −1.9 | 0.02 |
| | Immunoglobulin-like | | |
| Q8WTU5 | ATP/GTP-binding site motif A (P-loop) | −1.9 | 0.01 |
| ECHDC1 | Enoyl-CoA hydratase/isomerase | −1.9 | 0.02 |
| Q96JT2 | Acc: NM_033102]; prostein protein. [Source: RefSeq | −1.9 | 0.04 |
| Q9BZS9 | Acc: Q9BZS9]; PNAS-138. [Source: SPTREMBL | −1.9 | 0.00 |
| Q86UX6 | Protein kinase; Serine/Threonine protein kinase | −1.9 | 0.02 |
| SCN9A | ATP/GTP-binding site motif A (P-loop); Cation channel, | −1.9 | 0.04 |
| | non-ligand gated; IQ calmodulin-binding region; | | |
| | Polycystic kidney disease type 2 protein | | |
| BLVRB | Catalyzes electron transfer from reduced pyridine | −1.9 | 0.03 |
| | nucleotides to flavins. Possible role in protecting cells | | |
| | from oxidative damage or in regulating iron metabolism. | | |
| KIFAP3 | Involved in tethering the chromosomes to the spindle | −2.0 | 0.01 |
| | pole and in chromosome movement. | | |
| USP9Y | May function as a ubiquitin-protein or polyubiquitin | −2.0 | 0.01 |
| | hydrolase. | | |
| PAK4 | Activates the JNK pathway. | −2.0 | 0.02 |
| SLC39A1 | Mediates zinc uptake. May function as a major | −2.0 | 0.01 |
| | endogenous zinc uptake transporter in many cells of the | | |
| | body. | | |
| Q8NCL8 | low complexity; signal peptide; transmembrane | −2.0 | 0.03 |
| Q8NA48 | testes development-related NYD-SP18. | −2.0 | 0.00 |
| Q96CY3 | Bipartite nuclear localization signal | −2.0 | 0.04 |
| JUB | Sugar transporter superfamily; Zn-binding protein, LIM | −2.0 | 0.02 |
| CSF1R | Receptor for CSF-1, protein tyrosine-kinase | −2.0 | 0.01 |
| ATP7B | Involved in the export of copper out of the cells, such as | −2.0 | 0.02 |
| | the efflux of hepatic copper into the bile | | |
| KIAA1244 | Essential component of the high affinity receptor for the | −2.0 | 0.03 |
| | general membrane fusion machinery and an important | | |
| | regulator of transport vesicle docking and fusion | | |
| HTR1D | One of the several different receptors serotonin. | −2.0 | 0.01 |
| C9orf114 | Bipartite nuclear localization signal; DUF171 | −2.0 | 0.01 |
| HIF3A | Basic helix-loop-helix dimerization domain bHLH; | −2.0 | 0.01 |
| | Nuclear translocator; PAS domain | | |
| ARL14 | Involved in protein trafficking; may modulate vesicle | −2.0 | 0.01 |
| | budding and uncoating within the Golgi apparatus | | |
| MEF2C | Transcription activator which binds specifically to the | −2.1 | 0.01 |
| | MEF2 element in the regulatory regions of many muscle- | | |
| | specific genes. | | |
| Q9P1V9 | low complexity | −2.1 | 0.05 |
| FRMPD1 | PDZ/DHR/GLGF domain; RA domain | −2.1 | 0.05 |
| O60592 | Neutrophil cytosol factor 2; Proline-rich extensin; SH3 | −2.1 | 0.02 |
| | domain; Sorbin-like; Zn-finger, C2H2 type | | |
| C6orf65 | coiled-coil | −2.1 | 0.06 |
| ASB4 | Ankyrin repeat and SOCS box protein 4; ASB-4 | −2.1 | 0.02 |
| Q8IY68 | low complexity | −2.1 | 0.06 |
| Q9BU59 | G-protein beta WD-40 repeat | −2.1 | 0.04 |
| COX5B | One of the nuclear-coded polypeptide chains of | −2.2 | 0.01 |
| | cytochrome c oxidase, the terminal oxidase in | | |
| | mitochondrial electron transport | | |
| COL4A3BP | Phosphorylates on Ser and Thr residues the Goodpasture | −2.2 | 0.03 |
| | autoantigen (in vitro). Isoform 2 seems to be less active | | |
| RRH | May play a role in rpe physiology either by detecting | −2.2 | 0.01 |
| | light directly or by monitoring the concentration of | | |
| | retinoids or other photoreceptor-derived compounds | | |
| Q8TCQ1 | Bipartite nuclear localization signal; Zn-finger, RING | −2.3 | 0.04 |
| KLRG1 | C-type lectin | −2.3 | 0.05 |
| ZNF385 | Zn-finger, C2H2 matrin type; Zn-finger, C2H2 type | −2.3 | 0.05 |
| VAT1 | Synaptic vesicle membrane protein VAT-1 homolog | −2.3 | 0.03 |
| C14orf161 | transmembrane | −2.3 | 0.06 |
| HK1 | Hexokinase type I; HK I; Brain form hexokinase | −2.4 | 0.02 |
| EED | G-protein beta WD-40 repeat; Regulator of chromosome | −2.4 | 0.04 |
| | condensation, RCC1 | | |
| C22orf3 | Protein C22orf3 | −2.6 | 0.04 |
| PPT1 | Removes thioester-linked fatty acyl groups such as | −3.0 | 0.04 |
| | palmitate from modified cysteine residues in proteins or | | |
| | peptides during lysosomal degradation. | | |
| SCGB2A2 | Mammaglobin A precursor; Mammaglobin-1; | −3.1 | 0.02 |
| | Secretoglobin family 2A member 2 | | |
| O75915 | Prenylated rab acceptor PRA1 | −3.4 | 0.00 |
| Q86VG1 | Bipartite nuclear localization signal; NF-X1 type; Zn- | −4.7 | 0.04 |
| | finger, RING | | |

TABLE 71
Gene profiling of differentially expressed genes in human monocytes due to the
presence of bacterial endotoxin (LPS) and SEQ ID NO: 7 revealing 1012 differentially
expressed genes.

| | | Fold | |
|---|---|---|---|
| | | Change by | |
| Gene Name | Gene Description | LPS + SEQ 7 | p-value |
| RBP1 | Intracellular transport of retinol | 125.8 | 0.05 |
| TMOD4 | Blocks the elongation and depolymerization of the actin | 115.9 | 0.04 |
| | filaments at the pointed end. | | |
| Q8WUC6 | Bipartite nuclear localization signal; Class I peptide | 104.7 | 0.05 |
| | chain release factor domain | | |
| GPD1 | Glycerol-3-phosphate dehydrogenase [NAD+], | 89.7 | 0.04 |
| | cytoplasmic | | |
| KCNH7 | Pore-forming α-subunit of voltage-gated potassium | 80.6 | 0.06 |
| | channel. Channel properties may be modulated by cAMP | | |
| O43300 | Leucine-rich repeat | 77.1 | 0.03 |
| TGM4 | Associated with the mammalian reproductive process. | 73.5 | 0.04 |
| | Catalyzes the cross-linking of proteins and the | | |
| | conjugation of polyamines to specific proteins in the | | |
| | seminal tract | | |
| POU1F1 | Transcription factor involved in the specification of the | 64.2 | 0.03 |
| | lactotrope, somatotrope, and thyrotrope phenotypes in | | |
| | the developing anterior pituitary. Activates growth | | |
| | hormone and prolactin genes. Specifically binds to the | | |
| | consensus sequence 5′-TAAAT-3′; | | |
| FOXP1 | Transcriptional repressor that play an important role in | 60.4 | 0.04 |
| | the specification and differentiation of lung epithelium | | |
| KCNK6 | Exhibits outward rectification in a physiological K(+) | 58.8 | 0.05 |
| | gradient and mild inward rectification in symmetrical | | |
| | K(+) conditions | | |
| Q9C098 | Protein kinase; Serine/Threonine protein kinase | 57.1 | 0.01 |
| RHBDF1 | Rhomboid-like protein | 54.7 | 0.04 |
| Q8N135 | ATP/GTP-binding site motif A (P-loop); | 53.9 | 0.06 |
| | PDZ/DHR/GLGF domain | | |
| CD226 | Immunoglobulin-like | 53.2 | 0.03 |
| O43348 | Argininosuccinate synthase | 51.7 | 0.02 |
| SMF | SMF protein | 51.1 | 0.05 |
| Q9Y4T9 | low complexity | 48.3 | 0.05 |
| Q86WW9 | ATP/GTP-binding site motif A (P-loop); Lipoxygenase, | 47.2 | 0.02 |
| | LH2 domain | | |
| DLX5 | Homeobox protein DLX-5 | 43.2 | 0.05 |
| SMURF2 | E3 ubiquitin-protein ligase which accepts ubiquitin from | 42.1 | 0.04 |
| | an E2 ubiquitin-conjugating enzyme in the form of a | | |
| | thioester and then directly transfers the ubiquitin to | | |
| | targeted substrates. Interacts with SMAD1, SMAD2 and | | |
| | SMAD7 in order to trigger their ubiquitination and | | |
| | proteasome-dependent degradation. | | |
| CNTN5 | Fibronectin, type III; Immunoglobulin-like | 40.5 | 0.05 |
| ZNF73 | ATP/GTP-binding site motif A (P-loop); KRAB box; | 36.4 | 0.03 |
| | Zn-finger, C2H2 subtype; | | |
| ARNT | Required for activity of the Ah (dioxin) receptor. This | 35.7 | 0.05 |
| | protein is required for the ligand-binding subunit to | | |
| | translocate from the cytosol to the nucleus after ligand | | |
| | binding. | | |
| BNIP1 | Implicated in the suppression of cell death. Interacts with | 34.9 | 0.04 |
| | the BCL-2 and adenovirus E1B 19 kDa proteins | | |
| ITGA8 | Integrin alpha-8/beta-1 is a receptor for fibronectin and | 34.0 | 0.06 |
| | cytotactin. It recognizes the sequence R-G-D in its | | |
| | ligands | | |
| ZNF302 | May function as a transcription factor | 33.6 | 0.04 |
| GFER | Augmenter of liver regeneration (hERV1 protein). | 32.7 | 0.03 |
| NR2F2 | Regulation of the apolipoprotein A-I gene transcription. | 32.7 | 0.03 |
| | Binds to DNA site A | | |
| STATH | Salivary protein that stabilizes saliva supersaturated with | 31.8 | 0.03 |
| | Ca²⁺ salts by inhibiting the precipitation of calcium | | |
| | phosphate salts. | | |
| Q9H697 | limkain beta 2. | 29.4 | 0.05 |
| IDUA | Alpha-L-iduronidase precursor | 28.7 | 0.03 |
| WBSCR18 | Williams-Beuren syndrome chromosome region 18 | 27.4 | 0.06 |
| | protein | | |
| DNAJC1 | DnaJ homolog subfamily C member 1 | 26.9 | 0.05 |
| NRXN1 | Neuronal cell surface protein that may be involved in | 25.9 | 0.04 |
| | cell recognition and cell adhesion. May mediate | | |
| | intracellular signaling | | |
| FEZ2 | Involved in axonal outgrowth and fasciculation | 25.8 | 0.02 |
| OR5U1 | Putative odorant receptor | 23.8 | 0.04 |
| ENSG00000162701 | DENN (AEX-3) domain; uDENN domain | 22.8 | 0.04 |
| MCART1 | Mitochondrial carrier triple repeat 1 | 22.8 | 0.05 |
| SBNO1 | Helicase, C-terminal; RNA-binding region RNP-1 | 22.2 | 0.05 |
| CHRNA1 | Acetyl choline receptor. After binding acetylcholine, the | 21.3 | 0.03 |
| | AChR leads to opening of an ion-conducting channel | | |
| | across the plasma membrane | | |
| HOOK2 | Probable cytoskeletal linker protein, which may be | 19.9 | 0.02 |
| | involved in tethering membrane bound organelles to the | | |
| | cytoskeleton | | |
| ENSG00000105849 | RNA polymerase Rpa43 subunit | 19.8 | 0.05 |
| Q8N6Q6 | unknown | 18.8 | 0.04 |
| FHL3 | Four and a half LIM domains protein 3; FHL-3; Skeletal | 18.4 | 0.02 |
| | muscle LIM-protein 2; SLIM 2 | | |
| CKMT1 | Reversibly catalyzes the transfer of phosphate between | 17.8 | 0.04 |
| | ATP and various phosphogens (e.g. creatine phosphate). | | |
| | Creatine kinase isoenzymes play a central role in energy | | |
| | transduction. | | |
| Q8N8U9; | Trypsin inhibitor-like, cysteine-rich TIL region; Vitamin | 17.7 | 0.05 |
| Q8TF36 | K-dependent carboxylation/gamma-carboxyglutamic | | |
| | (GLA) domain; von Willebrand factor, type C, D | | |
| BLZF1 | basic leucine zipper nuclear factor 1. | 17.6 | 0.05 |
| Q9BRK2 | Protein of unknown function DUF625 | 17.6 | 0.04 |
| IDH3G | Isocitrate dehydrogenase [NAD] subunit gamma, | 17.5 | 0.04 |
| | mitochondrial precursor; | | |
| IL17C | Stimulates the release of tumor necrosis factor alpha and | 17.4 | 0.05 |
| | IL-1 beta from the monocytic cell line THP-1 | | |
| Q9H6R7 | Coiled-coil; low complexity | 17.1 | 0.06 |
| OR5P2 | Putative odorant receptor. Could also be involved in taste | 17.1 | 0.05 |
| | perception | | |
| PLCG1 | phospholipase C-gamma is a major substrate for heparin- | 17.0 | 0.04 |
| | binding growth factor 1 (acidic fibroblast growth factor)- | | |
| | activated tyrosine kinase | | |
| Q8NHU6 | Bipartite nuclear localization signal; Maternal tudor | 16.4 | 0.04 |
| | protein | | |
| RNF24 | RING finger protein 24 | 16.2 | 0.03 |
| Q9H9X6 | Bipartite nuclear localization signal | 16.0 | 0.05 |
| MAP3K1 | Component of a protein kinase signal transduction | 15.9 | 0.04 |
| | cascade. Activates the ERK and JNK kinase pathways by | | |
| | phosphor-ylation of MAP2K1 and MAP2K4. Activates | | |
| | CHUK and IKBKB, the central protein kinases of the | | |
| | NFκB pathway | | |
| ALDOB | Fructose-bisphosphate aldolase B; Liver-type aldolase | 15.5 | 0.01 |
| Q96LW2 | Blue (type 1) copper domain; Protein kinase; | 15.3 | 0.04 |
| | Serine/Threonine protein kinase | | |
| EPB41L4B | Band 4.1-like protein 4B; EHM2 protein; FERM- | 13.7 | 0.04 |
| | containing protein CG1 | | |
| RCL1 | Plays a role in 40S-ribosomal-subunit biogenesis in the | 13.5 | 0.01 |
| | early pre-rRNA processing steps at sites A0, A1 and A2 | | |
| | that are required for proper maturation of the 18S RNA | | |
| PTGDS2; | Catalyzes the conversion of PGH2 to PGD2, a | 13.3 | 0.04 |
| PGDS | prostaglandin that is a potent inhibitor of platelet | | |
| | aggregation | | |
| RGS14 | Inhibits signal transduction by increasing GTPase | 13.1 | 0.05 |
| | activity of G protein α-subunits driving them into | | |
| | inactive GDP-bound form | | |
| CYLC2 | Cylicin II (Multiple-band polypeptide II). | 12.7 | 0.03 |
| GPR174 | Putative receptor for purines coupled to G-proteins | 12.4 | 0.04 |
| PMPCB | Cleaves presequences (transit peptides) from | 12.3 | 0.05 |
| | mitochondrial protein precursors | | |
| ANKRD5 | Ankyrin repeat domain protein 5 | 12.2 | 0.05 |
| SYNE1 | Involved in the maintenance of nuclear organization and | 12.2 | 0.05 |
| | structural integrity. Probable anchoring protein which | | |
| | theters the nucleus to the cytoskeleton. Connects nuclei | | |
| | to the cytoskeleton by interacting with the nuclear | | |
| | envelope and with F-actin in the cytoplasm | | |
| IL17B | Stimulates the release of tumor necrosis factor alpha and | 12.2 | 0.02 |
| | IL-1 beta from the monocytic cell line THP-1 | | |
| GTF2H1 | Component of the core-TFIIH basal transcription factor | 12.1 | 0.04 |
| | involved in nucleotide excision repair (NER) of DNA | | |
| | and, in complex with CAK, in transcription by RNA | | |
| | polymerase II | | |
| ARRB1 | Regulates beta-adrenergic receptor function. | 12.0 | 0.03 |
| RAB22A | Ras-related protein Rab-22A; Rab-22; Rab-31; Rab-22B | 11.9 | 0.04 |
| OSBPL7 | Oxysterol binding protein-related protein 7; ORP-7 | 11.9 | 0.01 |
| FBN1 | Fibrillins are structural components of 10-12 nm | 11.8 | 0.03 |
| | extracellular calcium-binding microfibrils, which occur | | |
| | either in association with elastin or in elastin-free | | |
| | bundles. | | |
| PTGER3 | Receptor for prostaglandin E2 (PGE2) | 11.8 | 0.03 |
| SIRT1 | NAD-dependent deacetylase, which regulates processes | 11.8 | 0.04 |
| | such as apoptosis and muscle differentiation by | | |
| | deacetylating key proteins. | | |
| STUB1 | TPR repeat; Zn-finger, modified RING | 11.8 | 0.05 |
| DUSP14 | Involved in the inactivation of MAP kinases. | 11.6 | 0.01 |
| | Dephosphorylates ERK, JNK and p38 MAP-kinases | | |
| KRTHA4 | Keratin, type I cuticular Ha4; Hair keratin, type I Ha4 | 11.4 | 0.00 |
| SACM1L | Synaptojanin, N-terminal | 10.9 | 0.06 |
| Q8N336 | Protein of unknown function DUF609 | 10.1 | 0.04 |
| O60384 | Zn-finger, C2H2 type | 9.9 | 0.05 |
| PTPRCAP | Protein tyrosine phosphatase receptor type C-associated | 9.7 | 0.04 |
| | protein; Lymphocyte phosphatase-associated | | |
| | phosphoprotein | | |
| UBE2N | The UBE2V2/UBE2N heterodimer catalyzes the | 9.6 | 0.04 |
| | synthesis of non-canonical poly-ubiquitin chains that are | | |
| | linked through Lys-63; doesn't lead to protein | | |
| | degradation by the proteasome. Mediates transcriptional | | |
| | activation of target genes. Plays a role in the control of | | |
| | progress through the cell cycle and differentiation. | | |
| Q8TDS9 | putative G-protein coupled receptor GPCR42. | 9.6 | 0.00 |
| LNX | E3 Ubiquitin ligase protein that mediates ubiquitination | 9.5 | 0.06 |
| | and subsequent proteasomal degradation of NUMB. | | |
| GSTZ1 | Bifunctional enzyme showing minimal glutathione- | 9.3 | 0.05 |
| | conjugating activity and low glutathione peroxidase | | |
| | activity | | |
| SLC27A6 | AMP-dependent synthetase and ligase | 9.2 | 0.05 |
| CNTN6 | ABC transporter; Fibronectin, type III; Immunoglobulin- | 9.2 | 0.00 |
| | like | | |
| MSX1 | Acts as a transcriptional repressor. May play a role in | 9.2 | 0.05 |
| | limb-pattern formation. Acts in cranofacial development | | |
| | and specifically in odontogenesis | | |
| Q8N4J6 | HMG-I and HMG-Y DNA-binding domain (A + T-hook); | 9.1 | 0.05 |
| | Pistil-specific extensin-like protein; Proline-rich extensin | | |
| MYL4 | Regulatory light chain of myosin. Does not bind calcium | 8.9 | 0.05 |
| ARF1 | GTP-binding protein involved in protein trafficking | 8.8 | 0.01 |
| | among different compartments. Modulates vesicle | | |
| | budding and uncoating within the Golgi complex. | | |
| Q8NHE2 | SF21 protein. | 8.7 | 0.04 |
| Q8N3K5 | Cysteine-rich flanking region, N-terminal; | 8.7 | 0.06 |
| | Immunoglobulin-like; Leucine-rich repeat; RNA-binding | | |
| | region RNP-1 | | |
| Q9H5P1 | Zn-finger, C-x8-C-x5-C-x3-H type | 8.6 | 0.02 |
| CDK7 | Cyclin-dependent kinase-7; CDK7 is the catalytic | 8.6 | 0.05 |
| | subunit of the CDK-activating kinase complex, a serine- | | |
| | threonine kinase. Involved in cell cycle control and in | | |
| | RNA transcription by RNA polymerase II. | | |
| Q7RTU0 | Basic helix-loop-helix dimerization domain bHLH | 8.4 | 0.06 |
| ZNF322B | Zn-finger, C2H2 type | 8.3 | 0.05 |
| MPP4 | May play a role in retinal photoreceptors development | 8.2 | 0.03 |
| ALOX5 | Arachidonate 5-lipoxygenase; 5-lipoxygenase; 5-LO | 8.1 | 0.03 |
| NSF | May participate in trafficking events that are associated | 8.0 | 0.06 |
| | with myogenesis, such as myoblast fusion and/or | | |
| | GLUT4 trafficking; Required for vesicle-mediated | | |
| | transport. | | |
| Q9NZ13 | Zn-finger, C2H2 type | 8.0 | 0.03 |
| PPAP2B | PA-phosphatase related phosphoesterase | 7.9 | 0.05 |
| TUBGCP6 | Gamma-tubulin complex is necessary for microtublule | 7.8 | 0.03 |
| | nucleation at the centrosome | | |
| Q9BUJ0 | Alpha/beta hydrolase; Esterase/lipase/thioesterase, active | 7.6 | 0.04 |
| | site | | |
| TRPM3 | Calcium channel mediating constitutive calcium ion | 7.5 | 0.02 |
| | entry. Its activity is increased by reduction in | | |
| | extracellular osmolarity, by store depletion and | | |
| | muscarinic receptor activation | | |
| Q96E44 | Beta and gamma crystallin; Nuclear protein SET | 7.4 | 0.06 |
| Q9P1G1 | signal peptide | 7.4 | 0.06 |
| CENTB2 | GTPase-activating protein for ADP ribosylation factor | 7.4 | 0.05 |
| | family | | |
| XRCC5 | Single stranded DNA-dependent ATP-dependent | 7.3 | 0.03 |
| | helicase. Has a role in chromosome translocation. | | |
| GIT2 | GTPase-activating protein for the ADP ribosylation | 7.2 | 0.05 |
| | factor family | | |
| Q9BYE9 | Cadherin | 6.9 | 0.05 |
| C15orf15 | Bipartite nuclear localization signal; Ribosomal protein | 6.9 | 0.04 |
| | L24E | | |
| RAD52 | Involved in double-stranded break repair. Plays a central | 6.9 | 0.05 |
| | role in genetic recombination and DNA repair | | |
| UBXD2 | UBX domain-containing protein 2 | 6.8 | 0.04 |
| Q86TW0 | Bipartite nuclear localization signal; Zn-finger, C-x8-C- | 6.8 | 0.04 |
| | x5-C-x3-H type | | |
| TCN1 | Vitamin B12-binding protein. Transports cobalamin into | 6.7 | 0.03 |
| | cells | | |
| CCS | Delivers copper to copper zinc superoxide dismutase | 6.7 | 0.05 |
| | (SOD1) | | |
| Q96CN5 | Leucine-rich repeat | 6.6 | 0.04 |
| FBXW5 | Cyclin-like F-box; G-protein beta WD-40 repeat | 6.5 | 0.05 |
| C21orf108 | Nucleolar preribosomal-associated protein 1 | 6.3 | 0.04 |
| SOX9 | Plays an important role in the normal skeletal | 6.1 | 0.05 |
| | development. May regulate the expression of other genes | | |
| | by acting as a transcription factor for these genes | | |
| SYT1 | May have a regulatory role in the membrane interactions | 6.1 | 0.06 |
| | during trafficking of synaptic vesicles at the active zone | | |
| | of the synapse; binds acidic phospholipidsand can bind | | |
| | to at least three additional proteins, neurexins, syntaxin | | |
| | and AP2 | | |
| SYT11 | May be involved in Ca(2+)-dependent exocytosis of | 6.1 | 0.06 |
| | secretory vesicles through Ca(2+) and phospholipid | | |
| | binding to the C2 domain or may serve as Ca(2+) | | |
| | sensors in the process of vesicular trafficking and | | |
| | exocytosis | | |
| MPHOSPH6 | M-phase phosphoprotein 6 | 6.0 | 0.03 |
| ZNF208 | KRAB box; Neutral zinc metallopeptidases, zinc-binding | 6.0 | 0.04 |
| | region; Zn-finger, C2H2 subtype | | |
| HNRPH2 | This protein is a component of the heterogenous nuclear | 6.0 | 0.05 |
| | ribonucleoprotein (hnRNP) complexes | | |
| C13orf1 | SPla/RYanodine receptor SPRY | 5.9 | 0.04 |
| NRG1 | Direct ligand for ERBB3 and ERBB4 tyrosine kinase | 5.8 | 0.02 |
| | receptors. The multiple isoforms perform diverse | | |
| | functions such as inducing growth and differentiation of | | |
| | epithelial, glial, neuronal, and skeletal muscle cells; | | |
| EIF2C4 | Plays an important role in the eukaryotic peptide chain | 5.7 | 0.05 |
| | initiation process | | |
| HOOK1 | Cytoskeletal linker protein, which may be involved in | 5.7 | 0.02 |
| | tethering membrane-bound organelles to the | | |
| | cytoskeleton. | | |
| REPS1 | May coordinate the cellular actions of activated EGF | 5.6 | 0.04 |
| | receptors and Ral-GTPases | | |
| HLA-J | Immunoglobulin-like; Immunoglobulin/major | 5.6 | 0.03 |
| | histocompatibility complex (MHC); MHC protein, class I | | |
| SYNGR2 | Synaptogyrin-2; Cellugyrin | 5.6 | 0.05 |
| GRTP1 | RabGAP/TBC domain; Somatotropin hormone | 5.6 | 0.05 |
| RNF41 | Zn-finger, RING | 5.5 | 0.03 |
| PTGIS | Catalyzes the isomerization of prostaglandin H2 to | 5.5 | 0.01 |
| | prostacyclin (=prostaglandin I2) | | |
| TFEC | Basic helix-loop-helix dimerization domain bHLH | 5.5 | 0.01 |
| C20orf108 | Protein C20orf108 | 5.5 | 0.03 |
| RPS7 | 40S ribosomal protein 57; 405 ribosomal protein S7; 58 | 5.4 | 0.04 |
| ANKMY1 | Ankyrin repeat and MYND domain protein 1; Testis- | 5.4 | 0.00 |
| | specific ankyrin-like protein 1; Zinc-finger MYND | | |
| | domain protein 13 | | |
| GUCY1B3 | Guanylate cyclase soluble, beta-1 chain; GCS-beta-1; | 5.4 | 0.01 |
| | Soluble guanylate cyclase small subunit; GCS-beta-3 | | |
| TIRAP | Adapter involved in the TLR4 signaling pathway in the | 5.4 | 0.01 |
| | innate immune response. Acts via IRAK2 and TRAF-6, | | |
| | leading to the activation of NF-kappa-B, MAPK1, | | |
| | MAPK3 and JNK, resulting in cytokine secretion and the | | |
| | inflammatory response | | |
| MRPS22 | Mitochondrial 28S ribosomal protein S22; S22mt; MRP- | 5.3 | 0.04 |
| | S22 | | |
| IFNA2 | Produced by macrophages, IFN-alpha have antiviral | 5.3 | 0.01 |
| | activities. Interferon stimulates the production of two | | |
| | enzymes: a protein kinase and an oligoadenylate | | |
| | synthetase | | |
| MUC11 | Actin-binding, actinin-type; Eukaryotic RNA | 5.3 | 0.02 |
| | polymerase II heptapeptide repeat | | |
| KCNA6 | Mediates the voltage-dependent potassium ion | 5.2 | 0.06 |
| | permeability of excitable membranes. | | |
| SPG6 | WW/Rsp5/WWP domain | 5.2 | 0.03 |
| Q96MA7 | coiled-coil; low complexity | 5.1 | 0.03 |
| CCL20 | Chemotactic factor that attracts lymphocytes and, | 5.1 | 0.00 |
| | slightly, neutrophils, but not monocytes. | | |
| C20orf26 | Protein C20orf26 | 5.1 | 0.01 |
| TG | Precursor of the iodinated thyroid hormones thyroxine | 5.0 | 0.05 |
| | (T4) and triiodothyronine (T3) | | |
| PAK2 | The activated kinase phosphorylates a variety of targets, | 5.0 | 0.06 |
| | e.g. ribosomal protein S6, histone H4 and myelin basic | | |
| | protein. | | |
| TREX2 | 26S proteasome-associated UCH37 interacting protein 1; | 4.9 | 0.02 |
| | X-linked protein STS1769 | | |
| TMSB10 | Plays an important role in the organization of the | 4.9 | 0.02 |
| | cytoskeleton. Binds to and sequesters actin monomers (G | | |
| | actin) and therefore inhibits actin polymerization | | |
| Q9BRX9 | G-protein beta WD-40 repeat | 4.9 | 0.04 |
| Q9NW81 | Leucine-rich repeat | 4.8 | 0.02 |
| FOXQ1 | Forkhead box protein Q1; Hepatocyte nuclear factor 3 | 4.8 | 0.01 |
| | forkhead homolog 1; | | |
| PCCB | Propionyl-CoA carboxylase beta chain, mitochondrial | 4.7 | 0.03 |
| | precursor; | | |
| Q9H7Y2 | low complexity | 4.7 | 0.03 |
| PMAIP1 | Phorbol-12-myristate-13-acetate-induced protein 1; | 4.7 | 0.00 |
| | Immediate-early-response protein APR | | |
| SNRPC | This protein is associated with snRNP U1 | 4.6 | 0.02 |
| Q969S1 | Mitochondrial substrate carrier | 4.5 | 0.01 |
| BAZ2A | May play a role in transcriptional regulation interacting | 4.5 | 0.01 |
| | with ISWI. May serve a specific role in maintaining or | | |
| | altering the chromatin structure of the rDNA locus | | |
| DACH2 | Bipartite nuclear localization signal; Transforming | 4.5 | 0.04 |
| | protein Ski | | |
| Wdr68; Han11 | WD-repeat protein 68; WD-repeat protein An11 | 4.5 | 0.05 |
| | homolog | | |
| CCL23 | Shows chemotactic activity for monocytes, resting T- | 4.5 | 0.05 |
| | lymphocytes, and neutrophils, but not for activated | | |
| | lymphocytes. | | |
| ARHGEF1 | Seems to play a role in the regulation of RhoA GTPase | 4.4 | 0.00 |
| | by guanine nucleotide-binding alpha-12 (GNA12) and | | |
| | alpha-13 (GNA13) subunits. Acts as GTPase-activating | | |
| | protein (GAP) for GNA12 and GNA13, and as guanine | | |
| | nucleotide exchange factor (GEF) for RhoA GTPase. | | |
| Q7Z620 | C2 domain | 4.4 | 0.04 |
| MGAT5B | Beta(1,6)-N-acetylglucosaminyltransferase V isoform 1 | 4.3 | 0.05 |
| BATF | Functions as negative regulator of AP-1 mediated | 4.3 | 0.02 |
| | transcription by binding to Jun proteins. Jun/B-ATF | | |
| | heterodimers bind DNA preferentially at the 12-O- | | |
| | tetradecanoylphorbol-13-acetate response element (TRE) | | |
| | (consensus: 5‘TGA[CG]TCA-3’) and weaker at the | | |
| | cAMP responsive region (CRE) (consensus: | | |
| | 5‘GTGACGT[AC][AG]-3’), but are transcriptionally | | |
| | inert | | |
| DF; PALM | May be involved in control of cell shape | 4.3 | 0.02 |
| SP3 | Binds to GT and GC boxes promoters elements. | 4.2 | 0.02 |
| | Probable transcriptional activator | | |
| DNMT2 | Its strong binding to DNA suggests that it may mark | 4.2 | 0.05 |
| | specific sequences in the genome by binding to DNA | | |
| | through the specific target-recognizing motif. Doesn't | | |
| | seem to be active as a DNA methyltransferase. | | |
| Q9NX89 | unknown | 4.1 | 0.04 |
| Q96AF2 | Protein kinase; Tyrosine protein kinase | 4.1 | 0.00 |
| CHCHD5 | Bipartite nuclear localization signal | 4.1 | 0.02 |
| Q9NXD2 | Bipartite nuclear localization signal | 4.1 | 0.01 |
| STIM1 | Possible adhesion molecule with a role in early | 4.1 | 0.03 |
| | hematopoiesis by mediating attachment to stromal cells. | | |
| | Influences the survival and/or proliferation of B cell | | |
| | precursors. Binding to cells requires Mn(2+) | | |
| OCLN; RPS27 | May play a role in the formation and regulation of the | 4.1 | 0.03 |
| | tight junction (TJ) paracellular permeability barrier | | |
| TNFSF5IP1 | Tumor necrosis factor superfamily, member 5-induced | 4.0 | 0.00 |
| | protein 1; HDCMC29P; HSPC260.; | | |
| Q96MX1 | down-regulated by Ctnnb1, a. | 4.0 | 0.03 |
| TDRD1 | Tudor domain-containing protein 1 | 4.0 | 0.04 |
| C13orf11 | coiled-coil; low complexity; signal peptide; | 4.0 | 0.01 |
| | transmembrane | | |
| CSTF1 | One of the multiple factors required for polyadenylation | 4.0 | 0.04 |
| | and 3′-end cleavage of mammalian pre-mRNAs. May be | | |
| | responsible for the interaction of CSTF with other factors | | |
| | to form a stable complex on the pre-mRNA | | |
| KIF1A | Motor for anterograde axonal transport of synaptic | 4.0 | 0.02 |
| | vesicle precursors | | |
| Q96T82 | signal peptide; transmembrane | 4.0 | 0.00 |
| ARID3A | Binds a VH promoter proximal site necessary for | 3.9 | 0.02 |
| | induced mu-heavy-chain transcription. | | |
| CCL7 | Chemotactic factor that attracts monocytes and | 3.9 | 0.00 |
| | eosinophils, but not neutrophils. This protein can bind | | |
| | heparin. Binds to CCR1, CCR2 and CCR3 | | |
| FTCD | Folate-dependent enzyme, that displays both transferase | 3.8 | 0.03 |
| | and deaminase activity. Serves to channel one-carbon | | |
| | units from formiminoglutamate to the folate pool | | |
| LRRN1 | Cysteine-rich flanking region, C-terminal; Fibronectin, | 3.8 | 0.03 |
| | type III; Immunoglobulin-like; Leucine-rich repeat | | |
| PTGS2 | May have a role as a major mediator of inflammation | 3.8 | 0.03 |
| | and/or a role for prostanoid signaling in activity- | | |
| | dependent plasticity | | |
| MATP | Melanocyte differentiation antigen. May transport | 3.8 | 0.06 |
| | substances required for melanin biosynthesis | | |
| Q7Z5V3 | Latrophilin receptor; Olfactomedin-like | 3.8 | 0.03 |
| Q8NG51 | Zn-finger, Ran-binding | 3.8 | 0.01 |
| Q86XN7 | Aldehyde dehydrogenase; Proline-rich extensin | 3.8 | 0.01 |
| ONECUT2 | Transcriptional activator. Activates the transcription of a | 3.8 | 0.02 |
| | number of liver genes such as HNF3B | | |
| GNS | N-acetylglucosamine-6-sulfatase precursor; | 3.7 | 0.04 |
| COLEC12 | Protein C2orf4; C21orf19-like protein | 3.7 | 0.01 |
| IL1A | Produced by activated macrophages, IL-1α stimulates | 3.7 | 0.00 |
| | thymocyte proliferation by inducing IL-2 release, B-cell | | |
| | maturation and proliferation, and fibroblast growth factor | | |
| | activity. IL-1 proteins are involved in the inflammatory | | |
| | response, being identified as endogenous pyrogens, and | | |
| | are reported to stimulate the release of prostaglandin and | | |
| | collagenase from synovial cells | | |
| TNFRSF9 | Receptor for TNFSF14/4-1BBL. Possibly active during | 3.7 | 0.02 |
| | T cell activation | | |
| SLC16A10 | T-type amino acid transporter 1; solute carrier family 16, | 3.7 | 0.04 |
| | # 10 | | |
| Q9H9V9 | Transcription factor jumonji, jmjC | 3.7 | 0.01 |
| Q9NXL6 | SID1 transmembrane family, member 1 | 3.7 | 0.04 |
| Q9NTI6 | low complexity | 3.7 | 0.03 |
| ASTN2 | Fibronectin, type III | 3.7 | 0.06 |
| DAZAP1 | Proline-rich extensin; RNA-binding region RNP-1 | 3.7 | 0.04 |
| RBM3 | Putative RNA-binding protein 3; | 3.6 | 0.02 |
| TEX14 | Ankyrin; Protein kinase | 3.6 | 0.04 |
| Q9H631 | Mak10 subunit, NatC N(alpha)-terminal | 3.6 | 0.05 |
| | acetyltransferase | | |
| C16orf3 | Protein C16orf3 | 3.6 | 0.06 |
| HYAL4 | EGF-like domain; Glycoside hydrolase, family 56; | 3.6 | 0.03 |
| | sperm surface protein PH20; Multicopper oxidase, type 1 | | |
| TACSTD1 | GA733 tumor-associated antigen gene family may | 3.6 | 0.04 |
| | function as growth factor receptors | | |
| PLA1A | Esterase/lipase/thioesterase, active site; | 3.6 | 0.01 |
| Q8IZ41; | ATP/GTP-binding site motif A (P-loop); Calcium- | 3.5 | 0.02 |
| Q96N04 | binding EF-hand; Ras GTPase superfamily | | |
| BACH1 | Transcriptional regulator that acts as repressor or | 3.5 | 0.03 |
| | activator. Binds, in-vitro, to NF-E2 binding sites. Play | | |
| | important roles in coordinating transcription activation | | |
| | and repression by MAFK | | |
| SULT1C1 | Catalyzes the sulfate conjugation of many drugs, | 3.5 | 0.02 |
| | xenobiotic compounds, hormones, and neurotransmitters. | | |
| KPNB1 | Functions in nuclear protein import, either in association | 3.5 | 0.05 |
| | with an adapter protein, like an importin-alpha subunit, | | |
| | which binds to nuclear localization signals (NLS) in | | |
| | cargo substrates, or by acting as autonomous nuclear | | |
| | transport receptor. Acting autonomously, serves itself as | | |
| | NLS receptor. | | |
| CCR7 | Receptor for the MIP-3β chemokine. Probable mediator | 3.5 | 0.02 |
| | of EBV effects on B lymphocytes or of normal | | |
| | lymphocyte functions | | |
| Q8NC34 | Immunoglobulin-like | 3.5 | 0.04 |
| GSK3B | Participates in the Wnt signaling pathway. Implicated in | 3.5 | 0.03 |
| | the hormonal control of several regulatory proteins | | |
| | including glycogen synthase, MYB and the transcription | | |
| | factor JUN. Phosphorylates JUN at sites proximal to its | | |
| | DNA-binding domain, thereby reducing its affinity for | | |
| | DNA | | |
| ACSL6 | Activation of long-chain fatty acids for both synthesis of | 3.5 | 0.03 |
| | cellular lipids, and degradation via beta-oxidation. | | |
| H2NC000011 | unknown | 3.5 | 0.04 |
| KLRB1 | C-type lectin | 3.5 | 0.03 |
| GPR30 | Orphan receptor; possibly for a chemokine | 3.4 | 0.02 |
| TNIP3 | Listeria induced gene; TNFAIP3 interacting protein 3 | 3.4 | 0.02 |
| DBI | Binds medium- and long-chain acyl-CoA esters with | 3.4 | 0.03 |
| | very high affinity and may function as an intracellular | | |
| | carrier of acyl-CoA esters. | | |
| PIP5K1B | Phosphatidylinositol-4-phosphate 5-kinase | 3.4 | 0.03 |
| Q92519 | Protein kinase | 3.4 | 0.02 |
| PKIG | Extremely potent competitive inhibitor of cAMP- | 3.4 | 0.03 |
| | dependent protein kinase activity, this protein interacts | | |
| | with the catalytic subunit of the enzyme after the cAMP- | | |
| | induced dissociation of its regulatory chains | | |
| STMN2 | May play a role in neuronal differentiation, and in | 3.3 | 0.03 |
| | modulating membrane interaction with the cytoskeleton | | |
| | during neurite outgrowth | | |
| NCK1 | Adapter protein which associates with tyrosine- | 3.3 | 0.04 |
| | phosphorylated growth factor receptors or their cellular | | |
| | substrates | | |
| ZFYVE20 | Zn-finger, C2H2 type, FYVE type | 3.3 | 0.03 |
| ATP2B1 | This magnesium-dependent enzyme catalyzes the | 3.3 | 0.00 |
| | hydrolysis of ATP coupled with the transport of calcium | | |
| | out of the cell | | |
| Q96PN6 | ATP/GTP-binding site motif A (P-loop); Guanylate | 3.3 | 0.03 |
| | cyclase | | |
| SOD2 | Destroys radicals which are normally produced within | 3.3 | 0.01 |
| | the cells and which are toxic to biological systems | | |
| VBP1 | Binds specifically to cytosolic chaperonin (c-CPN) and | 3.3 | 0.02 |
| | transfers target proteins to it. Binds to nascent | | |
| | polypeptide chain and promotes folding. | | |
| CXCL2 | Produced by activated monocytes and neutrophils and | 3.3 | 0.01 |
| | expressed at sites of inflammation. Hematoregulatory | | |
| | chemokine, which, in vitro, suppresses hematopoietic | | |
| | progenitor cell proliferation. | | |
| MADH7 | Antagonist of signaling by TGFβ (Transforming growth | 3.2 | 0.05 |
| | factor) type 1 receptor superfamily members; has been | | |
| | shown to inhibit TGFβ (Transforming growth factor) and | | |
| | activin signaling by associating with their receptors thus | | |
| | preventing SMAD2 access. Functions as an adaptor to | | |
| | recruit SMURF2 to the TGFβ receptor complex. | | |
| | SMAD7 is an inhibitory SMAD (I-SMAD) or | | |
| | antagonistic SMAD whose inhibitory activity is | | |
| | enhanced by SMURF2 | | |
| C21orf127 | Putative N6-DNA-methyltransferase; M.HsaHemK2P | 3.2 | 0.04 |
| CCDC5 | Coiled-coil domain containing 5 (spindle associated). | 3.2 | 0.01 |
| GCH1 | Isoform GCH-1 is the functional enzyme, enzymatically | 3.2 | 0.04 |
| | inactive isoforms may have other functions | | |
| BPAG1 | Cytoskeletal linker protein. Anchors keratin-containing | 3.2 | 0.05 |
| | intermediate filaments to the inner plaque of | | |
| | hemidesmosomes. The proteins may self-aggregate to | | |
| | form filaments or a two-dimensional mesh | | |
| DVL2 | May play a role in the signal transduction pathway | 3.1 | 0.06 |
| | mediated by multiple Wnt genes | | |
| GMEB2 | Trans-acting factor that binds to glucocorticoid | 3.1 | 0.05 |
| | modulatory elements (GME) present in the TAT | | |
| | (tyrosine aminotransferase) promoter and increases | | |
| | sensitivity to low concentrations of glucocorticoids. | | |
| MARK3 | Involved in the specific phosphorylation of microtubule- | 3.1 | 0.02 |
| | associated proteins for tau, MAP2 and MAP4. | | |
| | Phosphorylates CDC25C on Ser-216 | | |
| PHLDA2 | Pleckstrin-like | 3.1 | 0.01 |
| HERC2 | Cytochrome b5; Protein kinase; Regulator of | 3.1 | 0.02 |
| | chromosome condensation, RCC1; Zn-finger, ZZ type | | |
| PTS | Involved in the biosynthesis of tetrahydrobiopterin, an | 3.1 | 0.00 |
| | essential cofactor of aromatic amino acid hydroxylases. | | |
| HEY2 | Antifreeze protein, type I; Basic helix-loop-helix | 3.0 | 0.03 |
| | dimerization domain bHLH | | |
| NPR1 | Receptor for atrial natriuretic peptide. Has guanylate | 3.0 | 0.03 |
| | cyclase activity on binding of ANF | | |
| FMR2 | AF4/FMR2 family member 2; Fragile X mental | 3.0 | 0.02 |
| | retardation 2 syndrome protein; Ox19 protein; | | |
| Q8N958 | unknown | 3.0 | 0.00 |
| NEF3 | Neurofilaments usually contain three intermediate | 3.0 | 0.00 |
| | filament proteins: L, M, and H which are involved in the | | |
| | maintenance of neuronal caliber | | |
| SNTB2 | Adapter protein that binds to and probably organizes the | 3.0 | 0.02 |
| | subcellular localization of a variety of membrane | | |
| | proteins. May link various receptors to the actin | | |
| | cytoskeleton and the dystrophin glycoprotein complex. | | |
| | May play a role in the regulation of secretory granules | | |
| | via its interaction with PTPRN | | |
| SOD3 | Destroys radicals which are normally produced within | 3.0 | 0.03 |
| | the cells and which are toxic to biological systems | | |
| C21orf42 | Protein C21orf42 | 3.0 | 0.03 |
| EREG | May be a mediator of localized cell proliferation. As a | 2.9 | 0.01 |
| | mitogen it may stimulate cell proliferation and/or | | |
| | angiogenesis | | |
| OR1F2 | Putative odorant receptor | 2.9 | 0.02 |
| Q96HQ3 | low complexity | 2.9 | 0.03 |
| CCL2 | Chemotactic factor that attracts monocytes and basophils | 2.9 | 0.01 |
| | but not neutrophils or eosinophils. Has been implicated | | |
| | in the pathogenesis of diseases characterized by | | |
| | monocytic infiltrates, like psoriasis, rheumatoid arthritis | | |
| | or atherosclerosis. | | |
| METTL4 | Bipartite nuclear localization signal; MT-A70; N-6 | 2.9 | 0.03 |
| | Adenine-specific DNA methylase | | |
| O60290 | KRAB box | 2.9 | 0.04 |
| PLK4 | Protein kinase; Tyrosine protein kinase | 2.8 | 0.06 |
| COX7B | One of the polypeptide chains of cytochrome c oxidase, | 2.8 | 0.01 |
| | the terminal oxidase in mitochondrial electron transport | | |
| GNG2 | Guanine nucleotide-binding proteins (G proteins) are | 2.8 | 0.02 |
| | involved as modulators or transducers in various | | |
| | transmembrane signaling systems. The beta and gamma | | |
| | chains are required for the GTPase activity, for | | |
| | replacement of GDP by GTP, and for G protein-effector | | |
| | interaction | | |
| MTRF1 | Mitochondrial peptide chain release factor that directs | 2.8 | 0.04 |
| | the termination of translation in response to the peptide | | |
| | chain termination codons UAA and UAG | | |
| X59362.1 | Phospholipid hydroperoxide glutathione peroxidase, | 2.8 | 0.05 |
| | mitochondrial precursor (EC 1.11.1.12) (PHGPx) (GPX- | | |
| | 4). | | |
| Q96SU5 | low complexity; transmembrane | 2.8 | 0.03 |
| NFKB2 | NFκB subunits p52 and p100 are respectively the minor | 2.8 | 0.01 |
| | and major forms. Appear to have dual functions such as | | |
| | cytoplasmic retention of attached NFκB proteins and | | |
| | generation of p52 by a cotranslational processing. The | | |
| | proteasome-mediated process ensures the production of | | |
| | both p52 and p100 and preserves their independent | | |
| | function. p52 binds to the kappa-B consensus sequence | | |
| | 5′-GGRNNYYCC-3′, located in the enhancer region of | | |
| | genes involved in immune response and acute phase | | |
| | reactions. | | |
| EBF3 | Transcriptional activator which recognizes variations of | 2.8 | 0.05 |
| | the palindromic sequence 5′-ATTCCCNNGGGAATT- | | |
| | 3′; Transcriptional factor which recognizes variations of | | |
| | the palindromic sequence 5′-ATTCCCNNGGGAATT- | | |
| | 3′; | | |
| Q9H5A9 | 60S Acidic ribosomal protein | 2.8 | 0.00 |
| NICE1 | NICE-1 protein | 2.8 | 0.00 |
| ADRM1 | Promotes cell adhesion | 2.8 | 0.00 |
| ATP2B4 | This magnesium-dependent enzyme catalyzes the | 2.8 | 0.05 |
| | hydrolysis of ATP coupled with the transport of calcium | | |
| | out of the cell | | |
| Q86V45 | G-protein beta WD-40 repeat | 2.8 | 0.03 |
| REG1A | Might act as an inhibitor of spontaneous calcium | 2.8 | 0.05 |
| | carbonate precipitation. May be associated with neuronal | | |
| | sprouting in brain, and with brain and pancreas | | |
| | regeneration | | |
| PLAUR | Acts as a receptor for urokinase plasminogen activator. | 2.8 | 0.03 |
| | Plays a role in localizing and promoting plasmin | | |
| | formation. Mediates the proteolysis-independent signal | | |
| | transduction activation effects of U-PA. | | |
| PSMA1 | Proteasome subunit, a multicatalytic proteinase complex | 2.7 | 0.04 |
| | with ATP-dependent proteolytic activity. | | |
| G0S2 | Potential oncogene and regulator of latent HIV | 2.7 | 0.03 |
| ITPKB | Inositol 1,4,5-trisphosphate 3-kinase B; IP3K-B | 2.7 | 0.04 |
| PWP2H | Periodic tryptophan protein 2 homolog | 2.7 | 0.04 |
| Q9H8U7 | coiled-coil; low complexity | 2.7 | 0.02 |
| Q96BW9 | unknown | 2.7 | 0.05 |
| ZNF595 | KRAB box; Zn-finger, C2H2 subtype | 2.7 | 0.06 |
| MLLT3 | Protein AF-9; Myeloid/lymphoid or mixed-lineage | 2.7 | 0.01 |
| | leukemia translocated to chromosome 3 protein | | |
| PMSCL1 | Component of the nuclear exosome exoribonuclease | 2.7 | 0.04 |
| | complex. Required for the 3′ processing of the 7S pre- | | |
| | RNA to the mature 5.8S rRNA. Has a 3‘5’ exonuclease | | |
| | activity | | |
| UBCE7IP1 | Isoform 1 acts as an E3 ubiquitin ligase. Promotes | 2.7 | 0.06 |
| | degradation of TLR4 amd TLR9. Isoform 3/ZIN inhibits | | |
| | TNF and IL-1 mediated activation of NFκB. Promotes | | |
| | TNF and RIP mediated apoptosis. | | |
| CTLA4 | Possibly involved in T-cell activation. Binds to B7-1 | 2.7 | 0.01 |
| | (CD80) and B7-2 (CD86) | | |
| Q8N2S5 | Pistil-specific extensin-like protein; Proline-rich extensin | 2.7 | 0.05 |
| Q96EC8 | Protein of unknown function DUF649 | 2.7 | 0.05 |
| YWHAH | Adapter protein implicated in the regulation of a large | 2.6 | 0.03 |
| | spectrum of both general and specialized signaling | | |
| | pathways. Binds to a large number of partners, usually | | |
| | by recognition of a phosphoserine or phosphothreonine | | |
| | motif. Binding generally results in the modulation of the | | |
| | activity of the binding partner | | |
| MGAT5 | Catalyzes the addition of N-acetylglucosamine in beta 1- | 2.6 | 0.01 |
| | 6 linkage to the alpha-linked mannose of biantennary N- | | |
| | linked oligosaccharides. It is one of the most important | | |
| | enzymes involved in the regulation of the biosynthesis of | | |
| | glycoprotein oligosaccharides | | |
| DNAH5 | ATP/GTP-binding site motif A (P-loop); Dynein heavy | 2.6 | 0.04 |
| | chain; Eukaryotic thiol (cysteine) protease | | |
| SPG7 | 2Fe—2S ferredoxin; Peptidase M41 | 2.6 | 0.02 |
| INSIG1 | May play a role in growth and differentiation of tissues | 2.6 | 0.02 |
| | involved in metabolic control. May play a regulatory role | | |
| | during G0/G1 transition of cell growth | | |
| PANK3 | Plays a role in the physiological regulation of the | 2.6 | 0.02 |
| | intracellular CoA concentration | | |
| Q8WUC7 | Bipartite nuclear localization signal | 2.6 | 0.00 |
| HDC | Histidine decarboxylase; HDC | 2.6 | 0.04 |
| DGKA | Upon cell stimulation converts the second messenger | 2.6 | 0.03 |
| | diacylglycerol into phosphatidate, initiating the | | |
| | resynthesis of phosphatidylinositols and attenuating | | |
| | protein kinase C activity | | |
| HNF4A | Transcriptionally controlled transcription factor. Binds to | 2.6 | 0.01 |
| | DNA sites required for the transcription of alpha 1- | | |
| | antitrypsin, apolipoprotein CIII, transthyretin genes and | | |
| | HNF1-alpha. | | |
| MAP1LC3A | Probably involved in formation of autophagosomal | 2.5 | 0.04 |
| | vacuoles | | |
| DACH2 | Bipartite nuclear localization signal; Transforming | 2.5 | 0.04 |
| | protein Ski | | |
| ZNF80 | May be involved in transcriptional regulation | 2.5 | 0.02 |
| TNFAIP6 | Possibly involved in cell-cell and cell-matrix interactions | 2.5 | 0.00 |
| | during inflammation and tumorigenesis | | |
| HIF1A | Functions as a master transcriptional regulator of the | 2.5 | 0.04 |
| | adaptive response to hypoxia. Under hypoxic conditions | | |
| | activates the transcription of over 40 genes, including, | | |
| | erythropoietin, glucose transporters, glycolytic enzymes, | | |
| | vascular endothelial growth factor, and other genes | | |
| | whose protein products increase oxygen delivery or | | |
| | facilitate metabolic adaptation to hypoxia. Binds to core | | |
| | DNA sequence 5′-[AG]CGTG-3′; within the hypoxia | | |
| | response element (HRE) of target gene promoters. | | |
| | Activation requires recruitment of transcriptional | | |
| | coactivators such as CREBPB and EP300. Activity is | | |
| | enhanced by interaction with both, NCOA1 or NCOA2. | | |
| | Interaction with redox regulatory protein APEX seems to | | |
| | activate CTAD and potentiates activation by NCOA1 | | |
| | and CREBBP | | |
| SLC39A8 | Zinc transporter ZIP | 2.5 | 0.01 |
| MAFF | Interacts with the upstream promoter region of the | 2.5 | 0.00 |
| | oxytocin receptor gene. May be involved in the cellular | | |
| | stress response | | |
| CD22 | Mediates B-cell B-cell interactions. Binds sialylated | 2.5 | 0.06 |
| | glycoproteins; one of which is CD45. Preferentially | | |
| | binds to alpha2,6-linked sialic acid. Upon ligand induced | | |
| | tyrosine phosphorylation in the immune response seems | | |
| | to be involved in regulation of B cell antigen receptor | | |
| | signaling. Plays a role in positive regulation through | | |
| | interaction with Src family tyrosine kinases and may also | | |
| | act as an inhibitory receptor by recruiting cytoplasmic | | |
| | phosphatases via their SH2 domains that block signal | | |
| | transduction through dephosphorylation of signaling | | |
| | molecules | | |
| S100A12 | Calcitermin possesses antifungal activity against | 2.5 | 0.03 |
| | C. albicans and is also active against E. coli and | | |
| | P. aeruginosa but not L. monocytogenes and S. aureus | | |
| IL1F9 | Function as an agonist of NFκ B activation through the | 2.5 | 0.03 |
| | orphan IL-1-receptor-related protein 2. Could constitute | | |
| | part of an independent signaling system analogous to | | |
| | interleukin-1α, β receptor agonist and interleukin-1 | | |
| | receptor type I (IL-1R1), that is present in epithelial | | |
| | barriers and takes part in local inflammatory response | | |
| Q9Y3U6 | low complexity | 2.5 | 0.00 |
| SERPINB8 | Serpin B8; Cytoplasmic antiproteinase 2; CAP2; CAP-2; | 2.5 | 0.03 |
| | Protease inhibitor 8 | | |
| CEBPD | C/EBP is a DNA-binding protein that recognizes two | 2.5 | 0.05 |
| | different motifs: the CCAAT homology common to | | |
| | many promoters and the enhanced core homology | | |
| | common to many enhancers. Important transcriptional | | |
| | activator in the regulation of genes involved in immune | | |
| | and inflammatory responses, may play an important role | | |
| | in the regulation of the several genes associated with | | |
| | activation and/or differentiation of macrophages | | |
| ATP13A3 | Probable cation-transporting ATPase 13A3; ATPase | 2.5 | 0.03 |
| | family homolog up-regulated in senescence cells 1 | | |
| NAP1L3 | Nucleosome assembly protein 1-like 3 | 2.5 | 0.01 |
| KIAA1404 | Protein KIAA1404 | 2.5 | 0.04 |
| C6orf103 | IQ calmodulin-binding region | 2.5 | 0.01 |
| Q96DM7 | Cytochrome b5 | 2.4 | 0.04 |
| ID2 | ID (inhibitor of DNA binding) HLH proteins lack a basic | 2.4 | 0.00 |
| | DNA-binding domain but are able to form heterodimers | | |
| | with other HLH proteins, thereby inhibiting DNA | | |
| | binding. ID-2 may be an inhibitor of tissue-specific gene | | |
| | expression | | |
| BGLAP | Constitutes 1-2% of the total bone protein. It binds | 2.4 | 0.06 |
| | strongly to apatite and calcium | | |
| AQP9 | Forms a channel with a broad specificity, mediates | 2.4 | 0.00 |
| | passage of a wide variety of non-charged solutes | | |
| C5orf13 | Neuronal protein 3.1; p311 protein | 2.4 | 0.01 |
| PROZ | Appears to assist hemostasis by binding thrombin and | 2.4 | 0.05 |
| | promoting its association with phospholipid vesicles | | |
| COL3A1 | Collagen type III occurs in most soft connective tissues | 2.4 | 0.00 |
| | along with type I collagen | | |
| TNF | Tumour necrosis factor-α; Cytokine that binds to | 2.4 | 0.01 |
| | TNFRSF1A/TNFR1 and TNFRSF1B/TNFBR. It is | | |
| | mainly secreted by macrophages and can induce cell | | |
| | death of certain tumor cell lines. It is potent pyrogen | | |
| | causing fever by direct action or by stimulation of | | |
| | interleukin 1 secretion and is implicated in the induction | | |
| | of cachexia, Under certain conditions it can stimulate | | |
| | cell proliferation and induce cell differentiation | | |
| CD151 | Essential for the proper assembly of the glomerular and | 2.4 | 0.02 |
| | tubular basement membranes in kidney | | |
| RAB3B | Protein transport. Probably involved in vesicular traffic | 2.4 | 0.00 |
| TUSC4 | Bipartite nuclear localization signal | 2.4 | 0.01 |
| PRDM2 | May function as a DNA-binding transcription factor. | 2.4 | 0.03 |
| | Binds to the macrophage-specific TPA-responsive | | |
| | element (MTE) of the HMOX1 (heme oxygenase 1) gene | | |
| | and may act as a transcriptional activator of this gene | | |
| DEF6 | Calcium-binding EF-hand; Pleckstrin-like; Tropomyosin | 2.4 | 0.02 |
| Q9H864 | Bipartite nuclear localization signal | 2.4 | 0.05 |
| HRAS | Ras proteins bind GDP/GTP and possess intrinsic | 2.4 | 0.01 |
| | GTPase activity | | |
| SPTBN1 | Fodrin, which seems to be involved in secretion, | 2.4 | 0.05 |
| | interacts with calmodulin in a calcium-dependent manner | | |
| | and is thus candidate for the calcium-dependent | | |
| | movement of the cytoskeleton at the membrane | | |
| ETV5 | Binds to DNA sequences containing the consensus | 2.4 | 0.01 |
| | nucleotide core sequence GGAA | | |
| C7orf16 | Inhibits protein phosphatase-2A and protein | 2.4 | 0.00 |
| | phosphatase-1 | | |
| Q96IB9 | low complexity | 2.4 | 0.04 |
| ODZ1 | EGF-like domain; Laminin-type EGF-like domain; NHL | 2.3 | 0.00 |
| | repeat; Proline-rich region; Subtilase serine protease | | |
| OR7A5 | Putative odorant receptor | 2.3 | 0.00 |
| FIGNL1 | AAA ATPase, central region; AAA-protein subdomain; | 2.3 | 0.00 |
| | ATP/GTP-binding site motif A (P-loop) | | |
| Q9Y3B9 | Bipartite nuclear localization signal | 2.3 | 0.04 |
| SRPRB | Has GTPase activity. May mediate the membrane | 2.3 | 0.05 |
| | association of SR alpha | | |
| CCNB1IP1 | E3 ubiquitin ligase. Modulates cyclin B levels and | 2.3 | 0.01 |
| | participates in the regulation of cell cycle progression | | |
| | through the G2 phase. Overexpression causes delayed | | |
| | entry into mitosis | | |
| DSG3 | Component of intercellular desmosome junctions. | 2.3 | 0.02 |
| | Involved in the interaction of plaque proteins and | | |
| | intermediate filaments mediating cell-cell adhesion | | |
| C2orf6 | Mob1/phocein family | 2.3 | 0.03 |
| LILRB1 | Receptor for class I MHC antigens. Recognizes a broad | 2.3 | 0.00 |
| | spectrum of HLA-A, HLA-B, HLA-C and HLA-G | | |
| | alleles. Ligand binding results in inhibitory signals and | | |
| | down-regulation of the immune response. Engagement | | |
| | of LILRB1 present on natural killer cells or T-cells by | | |
| | class I MHC molecules protects the target cells from | | |
| | lysis. | | |
| O14950 | Calcium-binding EF-hand | 2.3 | 0.04 |
| ZNF576 | Zn-finger, C2H2 type | 2.3 | 0.02 |
| WTAP | Wilms' tumor 1-associating protein; Putative pre-mRNA | 2.3 | 0.01 |
| | splicing regulator female-lethal(2D) homolog | | |
| PTP4A3 | Prenyl group binding site (CAAX box); Tyrosine | 2.3 | 0.03 |
| | specific protein phosphatase and dual specificity protein | | |
| | phosphatase | | |
| ELF1 | Transcription factor that appears to be required for the T- | 2.3 | 0.03 |
| | cell-receptor-mediated trans activation of HIV-2 gene | | |
| | expression. Activates the LYN and mouse BLK | | |
| | promoters | | |
| GDA | Catalyzes the hydrolytic deamination of guanine, | 2.3 | 0.01 |
| | producing xanthine and ammonia | | |
| IL6 | IL-6 is a cytokine with a wide variety of biological | 2.3 | 0.03 |
| | functions: it plays an essential role in the final | | |
| | differentiation of B-cells into Ig-secreting cells, it | | |
| | induces myeloma and plasmacytoma growth, it induces | | |
| | nerve cells differentiation, in hepatocytes it induces acute | | |
| | phase reactants | | |
| Q9BWJ2 | unknown | 2.3 | 0.03 |
| Q8TF23 | BED finger; Cytochrome c heme-binding site; KRAB | 2.3 | 0.02 |
| | box; Zn-finger, C2H2 subtype | | |
| APTX | Histidine triad (HIT) protein; Zn-finger, C2H2 type | 2.3 | 0.03 |
| Q96LP3 | Leucine-rich repeat | 2.3 | 0.02 |
| MAB21L1 | Mab-21 protein | 2.3 | 0.01 |
| ATP1B1 | ATPase B subunit; This is the non-catalytic component | 2.3 | 0.00 |
| | of the active enzyme, which catalyzes the hydrolysis of | | |
| | ATP coupled with the exchange of Na(+) and K(+) ions | | |
| | across the plasma membrane. The beta subunit regulates, | | |
| | through assembly of alpha/beta heterodimers, the | | |
| | number of sodium pumps transported to the plasma | | |
| | membrane | | |
| Q9H095 | IQ calmodulin-binding region | 2.3 | 0.04 |
| ENSG00000110900 | CD9/CD37/CD63 antigen | 2.3 | 0.02 |
| Q86X05 | Hly-III related proteins | 2.3 | 0.00 |
| IBRDC2 | Zn-finger, RING; Zn-finger, cysteine-rich C6HC | 2.3 | 0.01 |
| PAPOLG | Responsible for the post-transcriptional adenylation of | 2.3 | 0.02 |
| | the 3′-terminal several small RNAs including signal | | |
| | recognition particle (SRP) RNA, nuclear 7SK RNA, U2 | | |
| | small nuclear RNA, and ribosomal 5S RNA | | |
| Q7Z5X7 | low complexity | 2.2 | 0.02 |
| EIF3S12 | Binds to the 40S ribosome and promotes the binding of | 2.2 | 0.01 |
| | methionyl-tRNAi and mRNA | | |
| MDN1 | May function as a nuclear chaperone in the assembly/ | 2.2 | 0.03 |
| | disassembly of macromolecular complexes in the | | |
| | nucleus | | |
| C6orf123 | Protein C6orf123; HGC6.2 protein | 2.2 | 0.03 |
| ADM | AM and PAMP are potent hypotensive and vasodilatator | 2.2 | 0.01 |
| | agents. Numerous actions have been reported most | | |
| | related to the physiologic control of fluid and electrolyte | | |
| | homeostasis. | | |
| PLAC8 | Placenta-specific gene 8 protein; C15 protein | 2.2 | 0.05 |
| CYP19A1 | Catalyzes the formation of aromatic C18 estrogens from | 2.2 | 0.03 |
| | C19 androgens | | |
| ENSG00000144872 | Ribosomal protein L39e | 2.2 | 0.00 |
| PPP1CB | Protein phosphatase (PP1) is essential for cell division, it | 2.2 | 0.02 |
| | participates in the regulation of glycogen metabolism, | | |
| | muscle contractility and protein synthesis. Involved in | | |
| | regulation of ionic conductances and long-term synaptic | | |
| | plasticity | | |
| TRIM36 | Butyrophylin-like; Fibronectin, type III; SPla/RYanodine | 2.2 | 0.05 |
| | receptor SPRY; Zn-finger, B-box, RING | | |
| TRPC7 | Thought to form a receptor-activated non-selective | 2.2 | 0.02 |
| | calcium permeant cation channel. Probably is operated | | |
| | by a phosphatidylinositol second messenger system | | |
| | activated by receptor tyrosine kinases or G-protein | | |
| | coupled receptors. Activated by diacylglycerol (DAG) | | |
| | (By similarity). May also be activated by intracellular | | |
| | calcium store depletion | | |
| Q15061 | G-protein beta WD-40 repeat | 2.2 | 0.03 |
| CACNA1H | Voltage-sensitive calcium channels (VSCC) mediate the | 2.2 | 0.01 |
| | entry of calcium ions into excitable cells and are also | | |
| | involved in a variety of calcium-dependent processes, | | |
| | including muscle contraction, hormone or | | |
| | neurotransmitter release, gene expression, cell motility, | | |
| | cell division and cell death. processing as well as in cell | | |
| | growth processes | | |
| Q8N9J0 | FUN14 family | 2.2 | 0.01 |
| TNFAIP8 | Tumor necrosis factor, alpha-induced protein 8 | 2.2 | 0.05 |
| C1orf24 | Niban protein | 2.2 | 0.04 |
| DUSP5 | Displays phosphatase activity toward several substrates. | 2.2 | 0.04 |
| | The highest relative activity is toward ERK1 | | |
| AP4B1 | Subunit of novel type of clathrin-or non-clathrin- | 2.2 | 0.05 |
| | associated protein coat involved in targeting proteins | | |
| | from the trans-Golgi network (TGN) to the endosomal- | | |
| | lysosomal system | | |
| Q96MN5 | unknown | 2.2 | 0.03 |
| EBI3 | Cytokine receptor, common beta/gamma chain; | 2.2 | 0.02 |
| | Fibronectin, type III; Long hematopoietin receptor, | | |
| | soluble alpha chain | | |
| PELI1 | Scaffold protein involved in the IL-1 signaling pathway | 2.2 | 0.01 |
| | via its interaction with the complex containing IRAK | | |
| | kinases and TRAF6. Required for NF-kappa-B activation | | |
| | and IL-8 gene expression in response to IL-1 | | |
| PRPF3 | Participates in pre-mRNA splicing. May play a role in | 2.2 | 0.03 |
| | the assembly of the U4/U5/U6 tri-snRNP complex | | |
| SLC7A13 | Amino acid permease-associated region; Amino acid/ | 2.2 | 0.04 |
| | polyamine transporter, family I | | |
| MAP2K2 | Catalyzes the concomitant phosphorylation of a | 2.2 | 0.03 |
| | threonine and a tyrosine residue in a Thr-Glu-Tyr | | |
| | sequence located in MAP kinases. Activates the ERK1 | | |
| | and ERK2 MAP kinases | | |
| DDX21 | Can unwind double-stranded RNA (helicase) and can | 2.2 | 0.00 |
| | fold or introduce a secondary structure to a single- | | |
| | stranded RNA (foldase). Functions as cofactor for c-Jun- | | |
| | activated transcription. Involved in rRNA processing | | |
| GCN5L1 | Biogenesis of lysosome-related organelles complex-1, | 2.2 | 0.01 |
| | subunit 1; BLOC-1 subunit 1; GCN5-like protein 1; | | |
| | RT14 protein | | |
| SERPINB8 | Inhibits urokinase-type plasminogen activator. | 2.2 | 0.02 |
| Q9UJA5 | Bipartite nuclear localization signal; Eukaryotic | 2.2 | 0.04 |
| | initiation factor 3, gamma subunit | | |
| CD81 | May play an important role in the regulation of | 2.1 | 0.00 |
| | lymphoma cell growth. Interacts with a 16-kDa Leu-13 | | |
| | protein to form a complex possibly involved in signal | | |
| | transduction. | | |
| BID | Induces ICE-like proteases and apoptosis. Counters the | 2.1 | 0.06 |
| | protective effect of Bcl-2 | | |
| OR8B8 | Putative odorant receptor; Putative odorant receptor. | 2.1 | 0.04 |
| | Could also be involved in taste perception | | |
| RBMS3 | Paraneoplastic encephalomyelitis antigen; RNA-binding | 2.1 | 0.00 |
| | region RNP-1 (RNA recognition motif) | | |
| UNC5B | ATP/GTP-binding site motif A (P-loop); Death domain; | 2.1 | 0.04 |
| | Immunoglobulin-like; Thrombospondin, type I; ZU5 | | |
| | domain | | |
| ENSG00000154511 | low complexity; transmembrane | 2.1 | 0.00 |
| ERCC6 | Is involved in the preferential repair of active genes. | 2.1 | 0.01 |
| | Presumed DNA or RNA unwinding function. | | |
| PL6 | PL6 protein; Placental protein 6; PP6 | 2.1 | 0.05 |
| IGHG3 | Ig alpha is the major immunoglobulin class in body | 2.1 | 0.00 |
| | secretions. It serves both to defend against local infection | | |
| | and to prevent access of foreign antigens to the general | | |
| | immunologic system | | |
| RIPK2 | Activates pro-caspase-1 and pro-caspase-8. Potentiates | 2.1 | 0.02 |
| | CASP-8-mediated apoptosis. Activates NFκB | | |
| HDAC4 | Responsible for the deacetylation of lysine residues on | 2.1 | 0.01 |
| | the N-terminal part of the core histones (H2A, H2B, H3 | | |
| | and H4). Histone deacetylation gives a tag for epigenetic | | |
| | repression and plays an important role in transcriptional | | |
| | regulation, cell cycle progression and developmental | | |
| | events. | | |
| SOD2 | Destroys radicals which are normally produced within | 2.1 | 0.00 |
| | the cells and which are toxic to biological systems | | |
| DDX39 | ATP/GTP-binding site motif A; DEAD/DEAH box | 2.1 | 0.03 |
| | helicase | | |
| ADORA2A | Receptor for adenosine. The activity of this receptor is | 2.1 | 0.03 |
| | mediated by G proteins which activate adenylyl cyclase | | |
| O15069 | Nascent polypeptide-associated complex NAC | 2.1 | 0.04 |
| CPD | Metallocarboxypeptidase D precursor, gp180 | 2.1 | 0.03 |
| Q9P233 | Calponin-like actin-binding; Eggshell protein; Leucine- | 2.1 | 0.03 |
| | rich repeat | | |
| TTLL3 | Tubulin tyrosine ligase-like protein 3; HOTTL | 2.1 | 0.05 |
| C16orf44 | BTB/POZ domain; Kelch repeat | 2.1 | 0.02 |
| Q9Y627 | Aldehyde dehydrogenase; Leucine-rich repeat | 2.1 | 0.03 |
| OAZIN | Inhibits antizyme-dependent ornithine decarboxylase | 2.1 | 0.03 |
| | degradation by binding to antizyme | | |
| RRBP1 | Acts as a ribosome receptor and mediates interaction | 2.1 | 0.03 |
| | between the ribosome and the endoplasmic reticulum | | |
| | membrane | | |
| BOK | Apoptosis regulator Bcl-2 protein, BH; BCL2-like | 2.1 | 0.03 |
| | apoptosis inhibitor | | |
| RANBP9 | Proline-rich extensin; Proline-rich region; | 2.1 | 0.03 |
| | SPla/RYanodine receptor SPRY | | |
| IL7R | Receptor for interleukin-7 | 2.1 | 0.02 |
| Q9NW83 | low complexity | 2.0 | 0.04 |
| FER1L3 | May play a role in membrane regeneration and repair | 2.0 | 0.03 |
| CD44 | Receptor for hyaluronic acid (HA). Mediates cell-cell | 2.0 | 0.01 |
| | and cell-matrix interactions. Adhesion with HA plays an | | |
| | important role in cell migration, tumor growth and | | |
| | progression. Also involved in lymphocyte activation, | | |
| | recirculation and homing, and in hematopoiesis. | | |
| ENSG00000187017 | ATP/GTP-binding site motif A (P-loop); Actin-binding | 2.0 | 0.06 |
| | WH2; Bipartite nuclear localization signal | | |
| Q8IVG4 | low complexity | 2.0 | 0.01 |
| KIAA0084 | Hypothetical protein KIAA0084; HA2022 | 2.0 | 0.04 |
| Q9UF01 | FGF receptor activating protein 1 | 2.0 | 0.01 |
| NEFH | Neurofilaments usually contain three intermediate | 2.0 | 0.03 |
| | filament proteins: L, M, and H which are involved in the | | |
| | maintenance of neuronal caliber. NF-H has an important | | |
| | function in mature axons that is not subserved by the two | | |
| | smaller NF proteins | | |
| FNDC5 | Fibronectin, type III | 2.0 | 0.00 |
| KIAA1533 | GRAM domain | 2.0 | 0.01 |
| ZWINT | May play a role in targeting HZW10 to the kinetochore | 2.0 | 0.06 |
| | at prometaphase. Part of the MIS12 complex, which may | | |
| | be fundamental for kinetochore formation and proper | | |
| | chromosome segregation during mitosis | | |
| PNRC1 | Nuclear receptor coactivator. May play a role in signal | 2.0 | 0.05 |
| | transduction | | |
| SLAMF7 | SLAM family member; 719A24. protein; CD2-like | 2.0 | 0.04 |
| | receptor activating cytotoxic cells. | | |
| KLK1 | Glandular kallikreins cleave Met-Lys and Arg-Ser bonds | 2.0 | 0.00 |
| | in kininogen to release Lys-bradykinin | | |
| Q9P0P9 | transmembrane | 2.0 | 0.01 |
| NEK1 | Phosphorylates serines and threonines, but also appears | 2.0 | 0.04 |
| | to have tyrosine kinase activity. Implicated in control of | | |
| | meiosis | | |
| Q9H6X1 | Proline-rich extensin; Proline-rich region | 2.0 | 0.03 |
| ARIH1 | Might act as an E3 ubiquitin-protein ligase, or as part of | 2.0 | 0.06 |
| | the E3 complex, which accepts ubiquitin from specific | | |
| | E2 ubiquitin-conjugating enzymes, such as | | |
| | UBE2L3/UBCM4, and then transfers it to substrates | | |
| ZFP90 | May function as a repressor or silencer protein, and most | 2.0 | 0.03 |
| | likely exerts its repressing activity upon zinc-dependent | | |
| | binding to DNA. May be involved in proper | | |
| | spermatogenesis by repressing the expression of genes | | |
| | unnecessary or incompatible with the maintenance of a | | |
| | haploid cell state | | |
| TRIM39 | Tripartite motif protein 39; RING finger protein 23; | 2.0 | 0.02 |
| | Testis-abundant finger protein | | |
| Q96NU6 | Bipartite nuclear localization signal; RhoGAP domain | 2.0 | 0.01 |
| IRAK3 | Death domain; Protein kinase | 2.0 | 0.03 |
| Q9NTF2 | Prenyl group binding site (CAAX box) | 2.0 | 0.03 |
| PIP5K3 | Supports the intracellular PIP pool and to a lesser extent, | 2.0 | 0.00 |
| | the PI 4,5-P(2) pool. It generates PIP from PI and, to a | | |
| | lesser extent, PI 4,5-P(2) from PI 4-P. There are | | |
| | indications that it phosphorylates the D-5 rather than the | | |
| | D-4 position. Has a role in endosome-related membrane | | |
| | trafficking | | |
| EIF3S7 | Binds to the 40S ribosome and promotes the binding of | 2.0 | 0.05 |
| | methionyl-tRNAi and mRNA. Associates with the | | |
| | subunit p170 of eIF-3 | | |
| NME1 | Major role in the synthesis of nucleoside triphosphates | 2.0 | 0.00 |
| | other than ATP | | |
| GDAP1L1 | Ganglioside-induced differentiation-associated protein 1- | 2.0 | 0.02 |
| | like 1; GDAP1-L1 | | |
| Q8NBH1 | unknown | 2.0 | 0.00 |
| TCF12 | Binds specifically to oligomers of E-box motifs. May | 2.0 | 0.03 |
| | play important roles during development of the nervous | | |
| | system as well as in other organ systems | | |
| Q8TEB0 | unknown | 2.0 | 0.04 |
| LY6G5C | C-type lectin | 2.0 | 0.01 |
| ZNF451 | May be involved in transcriptional regulation. | 2.0 | 0.03 |
| | Coactivator for steroid receptors | | |
| PLEKHF2 | Pleckstrin-like; Zn-finger, FYVE type | 2.0 | 0.04 |
| NDUFB4 | Transfer of electrons from NADH to the respiratory | 2.0 | 0.02 |
| | chain. The immediate electron acceptor for the enzyme is | | |
| | believed to be ubiquinone | | |
| ATCAY | Caytaxin; Ataxia Cayman type protein; BNIP-H | 2.0 | 0.01 |
| GP5 | The GPIb-V-IX complex functions as the von | 2.0 | 0.05 |
| | Willebrand factor receptor and mediates von Willebrand | | |
| | factor-dependent platelet adhesion to blood vessels. The | | |
| | adhesion of platelets to injured vascular surfaces in the | | |
| | arterial circulation is a critical initiating event in | | |
| | hemostasis | | |
| ETV4 | Ets-domain; PEA3-type ETS-domain transcription | 2.0 | 0.05 |
| | factor, N-terminal | | |
| TNFAIP3 | Interacts with NAF1 and inhibits TNF-induced NF- | 2.0 | 0.04 |
| | kappa-B-dependent gene expression by interfering with | | |
| | an RIP- or TRAF2-mediated transactivation signal. | | |
| | Inhibitor of programmed cell death. Has a role in the | | |
| | function of the lymphoid system and may contribute to | | |
| | the in vivo effects of TNFα. Has deubiquitinating | | |
| | activity that is directed towards Lys-48 or Lys-63-linked | | |
| | polyubiquitin chains | | |
| OASL | Does not have 2′;-5′-OAS activity, but binds double- | 2.0 | 0.01 |
| | stranded RNA and DNA | | |
| HBG1 | The epsilon chain is a beta-type chain of early | 2.0 | 0.04 |
| | mammalian embryonic hemoglobin | | |
| PAPOLB | Polymerase that creates the 3′ poly(A) tail of | 2.0 | 0.00 |
| | mRNA&apos; s. Also required for the | | |
| | endoribonucleolytic cleavage reaction at some | | |
| | polyadenylylation sites. May acquire specificity through | | |
| | interaction with a cleavage and polyadenylation | | |
| | specificity factor (CPSF) at its C-terminus | | |
| ADA | Adenosine/AMP deaminase; Adenosine/AMP deaminase | 1.9 | 0.03 |
| | active site | | |
| LAMA2 | Binding to cells via a high affinity receptor, laminin is | 1.9 | 0.02 |
| | thought to mediate the attachment, migration and | | |
| | organization of cells into tissues during embryonic | | |
| | development by interacting with other extracellular | | |
| | matrix components | | |
| F13A1 | Factor XIII is activated by thrombin and calcium ion to a | 1.9 | 0.03 |
| | transglutaminase that catalyzes the formation of gamma- | | |
| | glutamyl-epsilon-lysine cross-links between fibrin | | |
| | chains, thus stabilizing the fibrin clot. Also cross-link | | |
| | alpha-2-plasmin inhibitor, or fibronectin, to the alpha | | |
| | chains of fibrin | | |
| SIAT4C | It may catalyze the formation of the NeuAc-alpha-2,3- | 1.9 | 0.03 |
| | Gal-beta-1,3-GalNAc- or NeuAc-alpha-2,3-Gal-beta-1,3- | | |
| | GlcNAc-sequences found in terminal carbohydrate | | |
| | groups of glycoproteins and glycolipids. It may be | | |
| | involved in the biosynthesis of the sialyl Lewis X | | |
| | determinant | | |
| BTBD12 | BTB/POZ domain | 1.9 | 0.06 |
| APOBEC2 | Probable C to U editing enzyme whose physiological | 1.9 | 0.02 |
| | substrate is not yet known. Does not display detectable | | |
| | apoB mRNA editing. Has a low intrinsic cytidine | | |
| | deaminase activity | | |
| RIN2 | Ras effector protein. May function as an upstream | 1.9 | 0.04 |
| | activator and/or downstream effector for RAB5B in | | |
| | endocytic pathway. May function as a guanine | | |
| | nucleotide exchange (GEF) of RAB5B, required for | | |
| | activating the RAB5 proteins by exchanging bound GDP | | |
| | for free GTP | | |
| UFC1 | E2-like enzyme which forms an intermediate with UFM1 | 1.9 | 0.01 |
| | via a thioester linkage | | |
| ZNF83 | ATP/GTP-binding site motif A (P-loop); Bipartite | 1.9 | 0.01 |
| | nuclear localization signal; KRAB box; Zn-finger, C2H2 | | |
| | subtype; | | |
| Q86SU1 | Ubiquitin interacting motif | 1.9 | 0.05 |
| FRS2 | Insulin receptor substrate-1, PTB | 1.9 | 0.04 |
| IER3 | Radiation-inducible immediate-early gene IEX-1; | 1.9 | 0.00 |
| | Differentiation-dependent gene 2 protein; DIF-2 protein | | |
| COX11 | Exerts its effect at some terminal stage of cytochrome c | 1.9 | 0.01 |
| | oxidase synthesis, probably by being involved in the | | |
| | insertion of the copper B into subunit I | | |
| FBXO32 | Probably recognizes and binds to some phosphorylated | 1.9 | 0.02 |
| | proteins and promotes their ubiquitination and | | |
| | degradation during skeletal muscle atrophy | | |
| Q96QA0 | Mpv17/PMP22 | 1.9 | 0.01 |
| GALNT9 | Ricin B lectin domain | 1.9 | 0.04 |
| VPREB1 | Associates with the Ig-mu chain to form a molecular | 1.9 | 0.05 |
| | complex that is expressed on the surface of pre-B-cells | | |
| | and regulates Ig gene rearrangements in the early steps | | |
| | of B-cell differentiation | | |
| NCAM2 | May play important roles in selective fasciculation and | 1.9 | 0.04 |
| | zone-to-zone projection of the primary olfactory axons | | |
| SLC17A5 | General substrate transporter | 1.9 | 0.04 |
| PMM2 | Involved in the synthesis of the GDP-mannose and | 1.9 | 0.02 |
| | dolichol-phosphate-mannose required for a number of | | |
| | critical mannosyl transfer reactions | | |
| CDC42EP4 | Probably involved in the organization of the actin | 1.9 | 0.01 |
| | cytoskeleton. May act downstream of CDC42 to induce | | |
| | actin filament assembly leading to cell shape changes. | | |
| | Induces pseudopodia formation, when overexpressed in | | |
| | fibroblasts | | |
| HFE | Binds to transferrin receptor (TFR) and reduces its | 1.9 | 0.05 |
| | affinity for iron-loaded transferrin | | |
| HMG20A | HMG1/2 (high mobility group) box | 1.9 | 0.02 |
| GBP1 | Binds GTP, GDP and GMP | 1.9 | 0.01 |
| NOL8 | Bipartite nuclear localization signal; RNA-binding | 1.9 | 0.02 |
| | region RNP-1 (RNA recognition motif) | | |
| C10orf46 | Proline-rich region | 1.9 | 0.06 |
| LECT2 | Has a neutrophil chemotactic activity. Also a positive | 1.9 | 0.04 |
| | regulator of chondrocyte proliferation | | |
| Q96136 | transmembrane | 1.9 | 0.03 |
| Q9H3H7 | Brain my050 protein. | 1.9 | 0.05 |
| DAB1 | Adapter molecule functioning in neural development. | 1.9 | 0.01 |
| | May regulate SIAH1 activity | | |
| ZIC1 | May play a role in cerebellar development | 1.9 | 0.02 |
| SIPA1 | GTPase activator for the nuclear Ras-related regulatory | 1.9 | 0.04 |
| | proteins Rap1 and Rap2 in vitro, converting it to the | | |
| | putatively inactive GDP-bound state | | |
| EHHADH | eIF-2 functions in the early steps of protein synthesis by | 1.9 | 0.02 |
| | forming a ternary complex with GTP and initiator tRNA. | | |
| NSMAF | Couples the p55 TNF-receptor (TNF-R55/TNFR1) to | 1.9 | 0.01 |
| | neutral sphingomyelinase (N-SMASE). Specifically | | |
| | binds to the N-smase activation domain of TNF-R55. | | |
| | May regulate ceramide production by N-SMASE | | |
| DSPG3 | May have a role in bone formation and also in | 1.9 | 0.01 |
| | establishing the ordered structure of cartilage through | | |
| | matrix organization | | |
| NR4A3 | Binds to the B1A response-element | 1.9 | 0.05 |
| CHRM1 | The muscarinic acetylcholine receptor mediates various | 1.9 | 0.03 |
| | cellular responses, including inhibition of adenylate | | |
| | cyclase, breakdown of phosphoinositides and modulation | | |
| | of potassium channels through the action of G proteins. | | |
| | Primary transducing effect is PI turnover | | |
| Q9Y2F5 | low complexity | 1.9 | 0.02 |
| EDG2 | Receptor for lysophosphatidic acid (LPA), a mediator of | 1.9 | 0.01 |
| | diverse cellular activities. Seems to be coupled to the | | |
| | G(i)/G(0), G(12)/G(13), and G(q) families of | | |
| | heteromeric G proteins | | |
| Q9HBM0 | Plays a pivotal role in the establisment of adherens | 1.9 | 0.01 |
| | junctions and their maintenance in adult life | | |
| FPR1 | High affinity receptor for N-formyl-methionyl peptides, | 1.9 | 0.02 |
| | which are powerful neutrophils chemotactic factors. | | |
| | Binding of FMLP to the receptor causes activation of | | |
| | neutrophils. This response is mediated via a G-protein | | |
| | that activates a phosphatidylinositol-calcium second | | |
| | messenger system | | |
| ATF1 | This protein binds the cAMP response element (CRE), a | 1.9 | 0.05 |
| | sequence present in many viral and cellular promoters. | | |
| | Binds to the Tax-responsive element (TRE) of HTLV-I. | | |
| | Mediates PKA-induced stimulation of CRE-reporter | | |
| | genes | | |
| ZCCHC2 | Zinc finger CCHC domain containing protein 2 | 1.9 | 0.03 |
| MANBAL | Protein MANBAL | 1.9 | 0.01 |
| KIF13A | Plus end-directed microtubule-dependent motor protein | 1.9 | 0.05 |
| | involved in mannnose-6-phosphate receptor (M6PR) | | |
| | transport to the plasma membrane | | |
| CYorf15A | Testis protein (Fragment). | 1.9 | 0.01 |
| ARHGDIA | Regulates the GDP/GTP exchange reaction of the Rho | 1.9 | 0.00 |
| | proteins by inhibiting the dissociation of GDP from | | |
| | them, and the subsequent binding of GTP to them | | |
| PCDHB10 | Potential calcium-dependent cell-adhesion protein. May | 1.8 | 0.01 |
| | be involved in the establishment and maintenance of | | |
| | specific neuronal connections in the brain | | |
| TRPC5 | Thought to form a receptor-activated non-selective | 1.8 | 0.00 |
| | calcium permeant cation channel. Probably is operated | | |
| | by a phosphatidylinositol second messenger system | | |
| | activated by receptor tyrosine kinases or G-protein | | |
| | coupled receptors. May also be activated by intracellular | | |
| | calcium store depletion | | |
| ZDHHC2 | Palmitoyltransferase specific for GAP43 and | 1.8 | 0.00 |
| | DLG4/PSD95 | | |
| Q8NEZ3 | 7-Fold repeat in clathrin and VPS proteins; G-protein | 1.8 | 0.04 |
| | beta WD-40 repeat | | |
| COQ7 | Potential central metabolic regulator | 1.8 | 0.01 |
| DDX3Y | Probable ATP-dependent RNA helicase. May play a role | 1.8 | 0.05 |
| | in spermatogenesis | | |
| ITPKA | Inositol 1,4,5-trisphosphate 3-kinase A; IP3K-A | 1.8 | 0.02 |
| GDF9 | Required for ovarian folliculogenesis | 1.8 | 0.04 |
| ZNF593 | Negatively modulates the DNA binding activity of Oct-2 | 1.8 | 0.03 |
| | and therefore its transcriptional regulatory activity. May | | |
| | also be a modulator of other octamer-binding proteins | | |
| SERTAD1 | Acts at E2F-responsive promoters to integrate signals | 1.8 | 0.01 |
| | provided by PHD- and/or bromodomain-containing | | |
| | transcription factors. Stimulates E2F-1/DP-1 | | |
| | transcriptional activity. Renders the activity of cyclin | | |
| | D1/CDK4 resistant to the inhibitory effects of | | |
| | p16(INK4a) | | |
| SLC22A11 | General substrate transporter | 1.8 | 0.04 |
| ZNF578 | KRAB box; Zn-finger, C2H2 subtype | 1.8 | 0.03 |
| TROAP | Could be involved with bystin and trophinin in a cell | 1.8 | 0.05 |
| | adhesion molecule complex at the time of the embryo | | |
| | implantation | | |
| HAPLN1 | Stabilizes the aggregates of proteoglycan monomers with | 1.8 | 0.02 |
| | hyaluronic acid in the extracellular cartilage matrix | | |
| ETS2 | C-ets-2 protein | 1.8 | 0.04 |
| Q96PY3 | Leucine-rich repeat | 1.8 | 0.04 |
| DNAJB6 | DnaJ homolog subfamily B member 6; Heat shock | 1.8 | 0.05 |
| | protein J2; | | |
| STAMBP | Bipartite nuclear localization signal; Mov34 family | 1.8 | 0.05 |
| PIP5K2C | Phosphatidylinositol-4-phosphate 5-kinase | 1.8 | 0.03 |
| RNH | Inhibitor of pancreatic RNase and angiogenin. May also | 1.8 | 0.01 |
| | function in the modulation of cellular activities | | |
| Q8N1W2 | Cytochrome c heme-binding site; Zn-finger, C2H2 type | 1.8 | 0.06 |
| Q96AP0 | low complexity | 1.8 | 0.01 |
| Q9H6L9 | Autophagocytosis associated protein | 1.8 | 0.04 |
| TSGA10 | Testis specific, 10. | 1.8 | 0.01 |
| SLC7A5 | Sodium-independent, high-affinity transport of large | 1.8 | 0.01 |
| | neutral amino acids. Involved in cellular amino acid | | |
| | uptake | | |
| ATR | Phosphatidylinositol 3- and 4-kinase-related, FAT, | 1.8 | 0.01 |
| | FATC; | | |
| ADRB2 | Beta-adrenergic receptors mediate the catecholamine- | 1.8 | 0.05 |
| | induced activation of adenylate cyclase through the | | |
| | action of G proteins. | | |
| 7NF585A | May be involved in transcriptional regulation | 1.8 | 0.01 |
| MKI67 | Thought to be required for maintaining cell proliferation | 1.8 | 0.04 |
| NPAS2 | Neuronal PAS domain protein 2; Neuronal PAS2; | 1.8 | 0.03 |
| | Member of PAS protein 4; MOP4 | | |
| WASF3 | Downstream effector molecules involved in the | 1.8 | 0.04 |
| | transmission of signals from tyrosine kinase receptors | | |
| | and small GTPases to the actin cytoskeleton | | |
| Q96IJ6 | Bacterial transferase hexapeptide repeat; Nucleotidyl | 1.8 | 0.02 |
| | transferase | | |
| Q8NBM8 | NULL | 1.8 | 0.02 |
| SIGLEC5 | Putative adhesion molecule that mediates sialic-acid | 1.8 | 0.03 |
| | dependent binding to cells. | | |
| EPHB2 | Receptor for members of the ephrin-B family | 1.8 | 0.03 |
| PIK4CB | Phosphorylates phosphatidylinositol (PI) in the first | 1.8 | 0.03 |
| | committed step in the production of the second | | |
| | messenger inositol-1,4,5,-trisphosphate (PIP). May | | |
| | regulate Golgi disintegration/reorganization during | | |
| | mitosis, possibly via its phosphorylation | | |
| BPAG1 | Cytoskeletal linker protein. Anchors keratin-containing | 1.8 | 0.01 |
| | intermediate filaments to the inner plaque of hemidesmosomes. | | |
| | May self-aggregate to form filaments or a 2D | | |
| | mesh | | |
| SERPINB1 | Regulates the activity of the neutrophil proteases | 1.8 | 0.03 |
| | elastase, cathepsin G and proteinase-3 | | |
| Q86T73 | von Willebrand factor, type A | 1.8 | 0.03 |
| FRMD1 | Band 4.1 domain | 1.8 | 0.00 |
| CD79A | Associated to surface IgM-receptor; may be involved in | 1.8 | 0.00 |
| | signal transduction | | |
| OXA1L | Required for the insertion of integral membrane proteins | 1.8 | 0.05 |
| | into the mitochondrial inner membrane. Essential for the | | |
| | activity and assembly of cytochrome oxidase | | |
| HSD3B1 | 3beta-HSD is a bifunctional enzyme that plays a crucial | 1.8 | 0.02 |
| | role in the biosynthesis of all classes of hormonal | | |
| | steroids | | |
| TBL1XR1 | F-box-like protein involved in the recruitment of the | 1.8 | 0.00 |
| | ubiquitin/19S proteasome complex to nuclear receptor- | | |
| | regulated transcription units. Plays an essential role in | | |
| | transcription activation mediated by nuclear receptors. | | |
| C21orf124 | Required for synthesis of pyridoxal-5-phosphate from | 1.8 | 0.01 |
| | vitamin B6 | | |
| ENSG00000166965 | Regulator of chromosome condensation, RCC1 | 1.8 | 0.03 |
| PTPN4 | May act at junctions between the membrane and the | 1.7 | 0.03 |
| | cytoskeleton | | |
| ODC1 | Ornithine decarboxylase; ODC | 1.7 | 0.05 |
| SLC23A3 | Xanthine/uracil/vitamin C permease family | 1.7 | 0.03 |
| KIAA0391 | 60S ribosomal protein L9; Hypothetical protein | 1.7 | 0.02 |
| | KIAA0391 | | |
| BZW2 | ATP/GTP-binding site motif A (P-loop); eIF4- | 1.7 | 0.01 |
| | γ/eIF5/eIF2-ε | | |
| MAPK6 | Phosphorylates microtubule-associated protein 2 | 1.7 | 0.01 |
| | (MAP2). May promote entry in the cell cycle | | |
| RSU1 | Potentially plays a role in the Ras signal transduction | 1.7 | 0.01 |
| | pathway. Capable of suppressing v-Ras transformation in | | |
| | vitro | | |
| PRDM10 | BTB/POZ domain; Zn-finger, C2H2 type | 1.7 | 0.03 |
| SHMT1 | Interconversion of serine and glycine | 1.7 | 0.01 |
| Q9Y4C1 | Transcription factor jumonji, jmjC | 1.7 | 0.03 |
| RAP1B | Ras-related protein Rap-1b; GTP-binding protein smg | 1.7 | 0.03 |
| | p21B | | |
| CASP9 | Involved in the activation cascade of caspases | 1.7 | 0.02 |
| | responsible for apoptosis. Binding of caspase-9 to Apaf- | | |
| | 1 leads to activation of the protease which then cleaves | | |
| | and activates caspase-3. Proteolytically cleaves | | |
| | poly(ADP-ribose) polymerase (PARP) | | |
| SDPR | Serum deprivation response protein; Phosphatidylserine- | 1.7 | 0.02 |
| | binding protein. | | |
| RBM8A | Part of a post-splicing multiprotein complex involved in | 1.7 | 0.04 |
| | both mRNA nuclear export and mRNA surveillance. | | |
| | Involved in nonsense-mediated decay (NMD) of mRNAs | | |
| | containing premature stop codons. Associates | | |
| | preferentially with mRNAs produced by splicing. Does | | |
| | not interact with pre-mRNAs, introns, or mRNAs | | |
| | produced from intronless cDNAs. Associates with both | | |
| | nuclear mRNAs and newly exported cytoplasmic | | |
| | mRNAs. Complex with MAGOH is a component of the | | |
| | nonsense mediated decay (NMD) pathway | | |
| Q9C0B6 | Peptidylprolyl isomerase, FKBP-type | 1.7 | 0.02 |
| CHRFAM7A | After binding acetylcholine, the AChR responds by an | 1.7 | 0.06 |
| | extensive change in conformation that affects all | | |
| | subunits and leads to opening of an ion-conducting | | |
| | channel across the plasma membrane | | |
| CYP3A4 | Cytochromes P450 are a group of heme-thiolate | 1.7 | 0.05 |
| | monooxygenases that perform a variety of oxidation | | |
| | reactions. | | |
| EHD1 | Acts in early endocytic membrane fusion and membrane | 1.7 | 0.05 |
| | trafficking of recycling endosomes | | |
| ANKH | Regulates intra- and extracellular levels of inorganic | 1.7 | 0.01 |
| | pyrophosphate (PPi), probably functioning as PPi | | |
| | transporter | | |
| PAX4 | Transcriptional repressor that binds to a common | 1.7 | 0.02 |
| | element in the glucagon, insulin and somatostatin | | |
| | promoters and plays an important role in the | | |
| | differentiation and development of pancreatic islet beta | | |
| | cells. | | |
| CACNA1B | Voltage-sensitive calcium channels (VSCC) mediate the | 1.7 | 0.01 |
| | entry of calcium ions into excitable cells and are also | | |
| | involved in a variety of calcium-dependent processes, | | |
| | including muscle contraction, hormone or | | |
| | neurotransmitter release, gene expression, cell motility, | | |
| | cell division and cell death. | | |
| STX11 | SNARE that acts to regulate protein transport between | 1.7 | 0.03 |
| | late endosomes and the trans-Golgi network | | |
| NTN4 | EGF-like domain; Laminin, N-terminal; Laminin-type | 1.7 | 0.04 |
| | EGF-like domain; Netrin, C-terminal | | |
| Q9NUK6 | Proteasome component region PCI | 1.7 | 0.01 |
| Q86UG6 | Olfactory receptor; Rhodopsin-like GPCR superfamily | 1.7 | 0.05 |
| LILRB2 | Receptor for class I MHC antigens. Recognizes a broad | 1.7 | 0.00 |
| | spectrum of HLA-A, HLA-B, HLA-C and HLA-G | | |
| | alleles. Involved in the down-regulation of the immune | | |
| | response and the development of tolerance. Competes | | |
| | with CD8A for binding to class I MHC antigens. Inhibits | | |
| | FCGR1A-mediated phosphorylation of cellular proteins | | |
| | and mobilization of intracellular calcium ions | | |
| NTRK2 | Receptor for brain-derived neurotrophic factor (BDNF), | 1.7 | 0.05 |
| | neurotrophin-3 and neurotrophin-4/5 but not nerve | | |
| | growth factor (NGF). Involved in the development | | |
| | and/or maintenance of the nervous system. This is a | | |
| | tyrosine-protein kinase receptor. Known substrates for | | |
| | the TRK receptors are SHC1, PI-3 kinase, and PLC- | | |
| | gamma-1 | | |
| Q8IYM2 | Protein of unknown function DUF467 | 1.7 | 0.01 |
| SART1 | SART-1 protein | 1.7 | 0.03 |
| EDG4 | Receptor for lysophosphatidic acid (LPA), a mediator of | 1.7 | 0.03 |
| | diverse cellular activities. Seems coupled to the | | |
| | G(i)/G(0), G(12)/G(13), and G(q) families of | | |
| | heteromeric G proteins | | |
| O14562 | Bipartite nuclear localization signal; Ubiquitin domain | 1.7 | 0.01 |
| Q8NC30 | transmembrane | 1.7 | 0.05 |
| PLEK | Major protein kinase C substrate of platelets, its exact | 1.7 | 0.05 |
| | function is not known | | |
| Q96C10 | DEAD/DEAH box helicase; Helicase, C-terminal | 1.7 | 0.01 |
| SLC30A5 | Cation efflux protein | 1.7 | 0.02 |
| RAX | Plays a critical role in eye formation by regulating the | 1.7 | 0.01 |
| | initial specification of retinal cells and/or their | | |
| | subsequent proliferation. | | |
| ABCE1 | Antagonizes the binding of 2-5A (5′-phosphorylated | 1.7 | 0.03 |
| | 2′,5′;-linked oligoadenylates) by RNase L through direct | | |
| | interaction with RNase L and therefore inhibits its | | |
| | endoribonuclease activity. May play a central role in the | | |
| | regulation of mRNA turnover. Antagonizes the anti-viral | | |
| | effect of the interferon-regulated 2-5A/RNase L pathway | | |
| DHX9 | Unwinds double-stranded DNA and RNA in a 3′ to 5′ | 1.7 | 0.05 |
| | direction. Alteration of secondary structure may | | |
| | subsequently influence interactions with proteins or other | | |
| | nucleic acids. Functions as a transcriptional activator | | |
| ZNF365 | Zn-finger, C2H2 type | 1.7 | 0.01 |
| GGN | Proline-rich extensin; Proline-rich region | 1.7 | 0.01 |
| PRRX1 | Acts as a transcriptional regulator of muscle creatine | 1.7 | 0.04 |
| | kinase (MCK) and so has a role in the establishment of | | |
| | diverse mesodermal muscle types. The protein binds to | | |
| | an A/T-rich element in the muscle creatine enhancer | | |
| SLC7A7 | Sodium-independent exchanger of cationic and large | 1.7 | 0.01 |
| | neutral amino acids | | |
| Q9BYH8 | Ankyrin | 1.7 | 0.03 |
| SMURF1 | E3 ubiquitin-protein ligase which accepts ubiquitin from | 1.7 | 0.03 |
| | an E2 ubiquitin-conjugating enzyme in the form of a | | |
| | thioester and then directly transfers the ubiquitin to | | |
| | targeted substrates. Interacts with receptor-regulated | | |
| | SMADs specific for the BMP pathway, SMAD1 and | | |
| | SMAD5, in order to trigger their ubiquitination and | | |
| | degradation and hence their inactivation | | |
| ICAM3 | ICAM proteins are ligands for the leukocyte adhesion | 1.7 | 0.03 |
| | LFA-1 protein (integrin alpha-L/beta-2). ICAM3 is also | | |
| | a ligand for integrin alpha-D/beta-2 | | |
| C7orf23 | Hypothetical protein C7orf23 | 1.7 | 0.05 |
| SLC35B3 | CGI-19 protein; chromosome 6 open reading frame 196. | 1.7 | 0.02 |
| | solute carrier family 35, member B3 | | |
| O00581 | D111/G-patch domain; Forkhead-associated (FHA); | 1.7 | 0.04 |
| | Proline-rich region; Ribosomal protein S5; | | |
| POLR2D | DNA-dependent RNA polymerase catalyzes the | 1.7 | 0.02 |
| | transcription of DNA into RNA using the four | | |
| | ribonucleoside triphosphates as substrates. Associates | | |
| | with POLR2G | | |
| Q9NUQ9 | NULL | 1.7 | 0.05 |
| STAU | Binds double-stranded RNA (regardless of the sequence) | 1.7 | 0.05 |
| | and tubulin. May play a role in specific positioning of | | |
| | mRNAs at given sites in the cell by crosslinking | | |
| | cytoskeletal and RNA components, and in stimulating | | |
| | their translation at the site | | |
| ZNF578 | KRAB box; Zn-finger, C2H2 subtype; | 1.7 | 0.03 |
| FBXO21 | Substrate-recognition component of the SCF (SKP1- | 1.7 | 0.05 |
| | CUL1-F-box protein)-type E3 ubiquitin ligase complex | | |
| Q96LX7 | Zn-finger, C2H2 type | 1.7 | 0.06 |
| MAPK12 | Responds to activation by environmental stress and pro- | 1.7 | 0.03 |
| | inflammatory cytokines by phosphorylating downstream | | |
| | targets. Plays a role in myoblast differentiation and also | | |
| | in the down-regulation of cyclin D1 in response to | | |
| | hypoxia in adrenal cells suggesting MAPK12 may | | |
| | inhibit cell proliferation while promoting differentiation | | |
| CLK3 | Phosphorylates serine- and arginine-rich (SR) proteins of | 1.7 | 0.05 |
| | the spliceosomal complex may be a constituent of a | | |
| | network of regulatory mechanisms that enable SR | | |
| | proteins to control RNA splicing. Phosphorylates serines, | | |
| | threonines and tyrosines | | |
| RAP2C | ATP/GTP-binding site motif A (P-loop); Prenyl group | 1.7 | 0.02 |
| | binding site (CAAX box); Ras GTPase superfamily | | |
| Q9ULQ0 | low complexity | 1.7 | 0.03 |
| SPATA13 | DH domain; Pleckstrin-like; Protamine P1; SH3 domain | 1.7 | 0.01 |
| GNA15 | Guanine nucleotide-binding proteins (G proteins) are | 1.7 | 0.05 |
| | involved as modulators or transducers in various | | |
| | transmembrane signaling systems | | |
| IL12B | Cytokine that can act as a growth factor for activated T | 1.7 | 0.00 |
| | and NK cells, enhance the lytic activity of | | |
| | NK/lymphokine-activated killer cells, and stimulate the | | |
| | production of IFN-gamma by resting PBMC | | |
| SSBP3 | May be involved in transcription regulation of the alpha | 1.7 | 0.05 |
| | 2(I) collagen gene where it binds to the single-stranded | | |
| | polypyrimidine sequences in the promoter region | | |
| ABCG4 | May be involved in macrophage lipid homeostasis | 1.7 | 0.01 |
| SLAMF1 | High-affinity self-ligand important in bidirectional T-cell | 1.7 | 0.04 |
| | to B-cell stimulation. SLAM-induced signal-transduction | | |
| | events in T lymphocytes are different from those in B | | |
| | cells. Two modes of SLAM signaling are likely to exist: | | |
| | one in which the inhibitor SH2D1A acts as a negative | | |
| | regulator and another in which protein-tyrosine | | |
| | phosphatase 2C-dependent signal transduction operates | | |
| C14orf166 | Protein C14orf166 | 1.7 | 0.02 |
| KPNA4 | Functions in nuclear protein import as an adapter protein | 1.7 | 0.03 |
| | for nuclear receptor KPNB1. | | |
| MXD3 | Basic helix-loop-helix dimerization domain bHLH | 1.6 | 0.04 |
| CPSF6 | Proline-rich extensin; Proline-rich region; RNA-binding | 1.6 | 0.00 |
| | region RNP-1 (RNA recognition motif) | | |
| HAS1 | Plays a role in hyaluronan/hyaluronic acid (HA) | 1.6 | 0.00 |
| | synthesis. Also able to catalyze the synthesis of chito- | | |
| | oligosaccharide depending on the substrate | | |
| SNX16 | May be involved in several stages of intracellular | 1.6 | 0.01 |
| | trafficking | | |
| URP2 | Probably involved in cell adhesion | 1.6 | 0.03 |
| CAPZB | F-actin capping proteins bind in a Ca(2+)-independent | 1.6 | 0.03 |
| | manner to the fast growing ends of actin filaments | | |
| | (barbed end) thereby blocking the exchange of subunits | | |
| | at these ends. Unlike other capping proteins (such as | | |
| | gelsolin and severin), these proteins do not sever actin | | |
| | filaments | | |
| Q8N4P3 | Metal-dependent phosphohydrolase, HD region | 1.6 | 0.05 |
| MAP1A | Structural protein involved in the filamentous cross- | 1.6 | 0.00 |
| | bridging between microtubules and other skeletal | | |
| | elements | | |
| CHC1 | Promotes the exchange of Ran-bound GDP by GTP. | 1.6 | 0.03 |
| | Involved in the regulation of onset of chromosome | | |
| | condensation in the S phase. Binds to the chromatin. | | |
| | RCC1/Ran complex (together with other proteins) acts as | | |
| | a component of a signal transmission pathway that | | |
| | detects unreplicated DNA | | |
| PLXNA4 | Cell surface receptor IPT/TIG; Plexin | 1.6 | 0.05 |
| CUL4A | Cullin | 1.6 | 0.03 |
| O94940 | SAM (and some other nucleotide) binding motif | 1.6 | 0.03 |
| ABCA2 | Probable transporter, unknown substrate. May have a | 1.6 | 0.05 |
| | role in macrophage lipid metabolism and neural | | |
| | development | | |
| C20orf135 | Protein C20orf135 | 1.6 | 0.06 |
| WASF1 | Downstream effector molecules involved in the | 1.6 | 0.05 |
| | transmission of signals from tyrosine kinase receptors | | |
| | and small GTPases to the actin cytoskeleton | | |
| MAPK8IP3 | The JNK-interacting protein (JIP) group of scaffold | 1.6 | 0.02 |
| | proteins selectively mediates JNK signaling by | | |
| | aggregating specific components of the MAPK cascade | | |
| | to form a functional JNK signaling module. May | | |
| | function as a regulator of vesicle transport, through | | |
| | interations with the JNK-signaling components and | | |
| | motor proteins | | |
| Q7M4L6 | SH2 motif | 1.6 | 0.01 |
| EPS15 | Involved in cell growth regulation. May be involved in | 1.6 | 0.04 |
| | the regulation of mitogenic signals and control of cell | | |
| | proliferation. Involved in the internalization of ligand- | | |
| | inducible receptors of the receptor tyrosine kinase (RTK) | | |
| | type, in particular EGFR | | |
| SFPQ | DNA- and RNA binding protein, involved in several | 1.6 | 0.00 |
| | nuclear processes. Essential pre-mRNA splicing factor | | |
| | required early in spliceosome formation and for splicing | | |
| | catalytic step II | | |
| ECGF1 | May have a role in maintaining the integrity of the blood | 1.6 | 0.01 |
| | vessels. Has growth promoting activity on endothelial | | |
| | cells, angiogenic activity in vivo and chemotactic | | |
| | activity on endothelial cells in vitro | | |
| PCBD | Involved in tetrahydrobiopterin biosynthesis. | 1.6 | 0.00 |
| CASP4 | Involved in the activation cascade of caspases | 1.6 | 0.04 |
| | responsible for apoptosis execution. Cleaves caspase-1 | | |
| HMGB1 | Binds preferentially single-stranded DNA and unwinds | 1.6 | 0.04 |
| | double stranded DNA | | |
| NUP62 | Interleukin-4-induced protein 1 precursor; FIG.-1 protein | 1.6 | 0.05 |
| GLG1 | Binds fibroblast growth factor and E-selectin (cell- | 1.6 | 0.06 |
| | adhesion lectin on endothelial cells mediates binding of | | |
| | neutrophils) | | |
| LILRA1 | May act as receptor for class I MHC antigens; May act | 1.6 | 0.02 |
| | as soluble receptor for class I MHC antigens | | |
| SUI1 | Necessary for scanning and involved in initiation site | 1.6 | 0.01 |
| | selection. Promotes the assembly of 48S ribosomal | | |
| | complexes at the authentic initiation codon of a | | |
| | conventional capped mRNA; Probably involved in | | |
| | translation | | |
| TGDS | dTDP-D-glucose 4,6-dehydratase | 1.6 | 0.01 |
| Q9NPI0 | low complexity; transmembrane | 1.6 | 0.02 |
| ZNF571 | KRAB box; Zn-finger, C2H2 subtype | 1.6 | 0.01 |
| FGF10 | Could be a growth factor active in the process of wound | 1.6 | 0.04 |
| | healing. Acts as a mitogen in the lung. May act in a | | |
| | manner similar to FGF-7 | | |
| Q8N7I3 | Immunoglobulin-like | 1.6 | 0.01 |
| GSG1 | germ cell associated 1 | 1.6 | 0.06 |
| KCNA10 | K+ channel tetramerisation; Kv channel; Shaker voltage- | 1.6 | 0.00 |
| | gated K+ channel | | |
| POLR1B | RNA polymerase beta subunit Rpb2, domain 2 | 1.6 | 0.05 |
| IL32; NK4 | May play a role in lymphocyte activation | 1.6 | 0.00 |
| O75121 | Immunoglobulin-like | 1.6 | 0.02 |
| Q9H9C7 | BRCT domain. | 1.6 | 0.00 |
| TNFRSF6 | Receptor for TNFSF6/FASL. The adapter molecule | 1.6 | 0.01 |
| | FADD recruits caspase-8 to the activated receptor. The | | |
| | resulting death-inducing signaling complex (DISC) | | |
| | performs caspase-8 proteolytic activation which initiates | | |
| | the subsequent cascade of caspases (aspartate-specific | | |
| | cysteine proteases) mediating apoptosis. FAS-mediated | | |
| | apoptosis may have a role in the induction of peripheral | | |
| | tolerance, in the antigen-stimulated suicide of mature T- | | |
| | cells, or both. The secreted isoforms 2 to 6 block | | |
| | apoptosis (in vitro) | | |
| EIF5A | The precise role of eIF-5A in protein biosynthesis is not | 1.6 | 0.04 |
| | known but it functions by promoting the formation of the | | |
| | first peptide bond | | |
| AREG | Bifunctional growth-modulating glycoprotein. Inhibits | 1.6 | 0.02 |
| | growth of several human carcinoma cells in culture and | | |
| | stimulates proliferation of human fibroblasts and certain | | |
| | other tumor cells | | |
| DYRK1B | Dual-specificity kinase which possesses both serine/ | 1.6 | 0.05 |
| | threonine and tyrosine kinase activities. Enhances the | | |
| | transcriptional activity of TCF1/HNF1A. Inhibits | | |
| | epithelial cell migration. | | |
| Q96M86 | Dynein heavy chain | 1.6 | 0.01 |
| KNS2 | Kinesin is a microtubule-associated force-producing | 1.6 | 0.00 |
| | protein that may play a role in organelle transport. The | | |
| | light chain may function in coupling of cargo to the | | |
| | heavy chain or in the modulation of its ATPase activity | | |
| SLC37A1 | Glycerol-3-phosphate transporter; G-3-P transporter; G- | 1.6 | 0.05 |
| | 3-P permease; Solute carrier family 37 member 1 | | |
| ICK | Protein kinase; Serine/Threonine protein kinase | 1.6 | 0.02 |
| Q8TEE6 | ATP/GTP-binding site motif A (P-loop); Peptidase | 1.6 | 0.00 |
| | family S16 | | |
| Q8IUZ5 | Aminotransferase class-III | 1.6 | 0.06 |
| HSPB1 | Involved in stress resistance and actin organization | 1.6 | 0.00 |
| NFYA | Stimulates the transcription of various genes by | 1.6 | 0.05 |
| | recognizing and binding to a CCAAT motif in | | |
| | promoters, for example in type 1 collagen, albumin and | | |
| | beta-actin genes | | |
| CLCA1 | H+-transporting two-sector ATPase, gamma subunit; | 1.6 | 0.03 |
| | von Willebrand factor, type A | | |
| SNTG1 | Adapter protein that binds to and probably organizes the | 1.6 | 0.02 |
| | subcellular localization of several proteins. May link | | |
| | various receptors to the actin cytoskeleton and the | | |
| | dystrophin glycol-protein complex. May participate in | | |
| | regulating the subcellular location of diacylglycerol | | |
| | kinase-zeta to ensure that diacyl-glycerol is rapidly | | |
| | inactivated following receptor activation | | |
| TNFRSF1B | Receptor with high affinity for TNFSF2/TNF-alpha and | 1.6 | 0.01 |
| | approximately 5-fold lower affinity for homotrimeric | | |
| | TNFSF1/lymphotoxin-alpha. The TRAF1/TRAF2 | | |
| | complex recruits the apoptotic suppressors BIRC2 and | | |
| | BIRC3 to TNFRSF1B/TNFR2. This receptor mediates | | |
| | most of the metabolic effects of TNF-alpha. Isoform 2 | | |
| | blocks TNF-alpha-induced apoptosis, which suggests | | |
| | that it regulates TNF-alpha function by antagonizing its | | |
| | biological activity | | |
| C20orf85 | Protein C20orf85 | 1.6 | 0.04 |
| Q8N1Q9 | Cation transporting ATPase, E1-E2 type | 1.6 | 0.02 |
| VGCNL1 | Cation channel, non-ligand gated; Ion transport protein | 1.6 | 0.03 |
| FPGT | Catalyzes the formation of GDP-L-fucose from GTP and | 1.6 | 0.00 |
| | L-fucose-1-phosphate. Functions as a salvage pathway to | | |
| | reutilize L-fucose arising from the turnover of | | |
| | glycoproteins and glycolipids | | |
| ENSG00000185305 | ATP/GTP-binding site motif A (P-loop) | 1.6 | 0.04 |
| PDCD1LG1 | Immunoglobulin-like | 1.6 | 0.05 |
| MFI2 | Involved in iron cellular uptake. Seems to be internalized | 1.6 | 0.01 |
| | and then recycled back to the cell membrane. | | |
| GK | Key enzyme in the regulation of glycerol uptake and | 1.6 | 0.01 |
| | metabolism | | |
| Q9BVN4 | Bacterial Sun/eukaryotic nucleolar Nop1/Nop2 | 1.6 | 0.06 |
| NPB | May be involved in the regulation of neuroendocrine | 1.6 | 0.03 |
| | system | | |
| VSX1 | Binds to the 37-bp core of the locus control region | 1.6 | 0.04 |
| | (LCR) of the red/green visual pigment gene cluster. May | | |
| | regulate the activity of LCR | | |
| Q96N98 | Amidase | 1.6 | 0.04 |
| Q96MB3 | Protein kinase | 1.6 | 0.04 |
| PIM2 | Serine/threonine-protein kinase Pim-2; Pim-2h | 1.6 | 0.03 |
| PHLDB1 | Forkhead-associated (FHA); Pleckstrin-like | 1.6 | 0.05 |
| ENSG00000188719 | unknown | 1.6 | 0.06 |
| ASB7 | Ankyrin repeat and SOCS box protein 7; ASB-7 | 1.6 | 0.01 |
| ENSG00000159752 | low complexity; signal peptide | 1.6 | 0.04 |
| GABRA6 | GABA, the major inhibitory neurotransmitter in the | 1.6 | 0.04 |
| | vertebrate brain. | | |
| NMI | May be involved in augmenting coactivator protein | 1.6 | 0.03 |
| | recruitment to a group of sequence-specific transcription | | |
| | factors. Augments cytokine-mediated STAT | | |
| | transcription. Enhances CBP/p300 coactivator protein | | |
| | recruitment to STAT1 and STAT5 | | |
| UNC5C | Death domain; Immunoglobulin-like; Thrombospondin, | 1.6 | 0.02 |
| | type I; ZU5 domain | | |
| Q7Z2R6 | low complexity; transmembrane | 1.6 | 0.02 |
| CNDP2; CN2; | Cytosolic nonspecific dipeptidase; Glutamate | 1.6 | 0.04 |
| CPGL | carboxypeptidase-like protein 1; CNDP dipeptidase 2 | | |
| SF3B5 | Pre-mRNA Splicing factor 3B subunit 5; 10 kDa subunit | 1.6 | 0.04 |
| DPYS | Dihydropyrimidinase; DHPase; Hydantoinase; DHP | 1.6 | 0.01 |
| DNAJA2 | Co-chaperone of Hsc70 | 1.6 | 0.03 |
| TPR | Component of the cytoplasmic fibrils of the nuclear pore | 1.6 | 0.03 |
| | complex implicated in nuclear protein import. Its N- | | |
| | terminus is involved in activation of oncogenic kinases | | |
| CD58 | Ligand of the T lymphocyte CD2 glycoprotein. This | 1.6 | 0.03 |
| | interaction is important in mediating thymocyte | | |
| | interactions with thymic epithelial cells, antigen- | | |
| | independent and -dependent interactions of T | | |
| | lymphocytes with target cells and antigen-presenting | | |
| | cells and the T lymphocyte rosetting with erythrocytes. | | |
| | In addition, the LFA-3/CD2 interaction may prime | | |
| | response by both the CD2+ and LFA-3+ cells | | |
| SLC22A11 | General substrate transporter | 1.5 | 0.05 |
| Q9P1G3 | Protein of unknown function DUF185 | 1.5 | 0.02 |
| TIMM8B | Mitochondrial import inner membrane translocase | 1.5 | 0.03 |
| | subunit TIM8 B; Deafness dystonia protein 2; DDP-like | | |
| | protein | | |
| Q86W75 | Sulfatase | 1.5 | 0.01 |
| Q9P2E5 | chondroitin sulfate glucuronyltransferase | 1.5 | 0.03 |
| USP13 | Ubiquitin carboxyl-terminal hydrolase 13; ISOT-3 | 1.5 | 0.05 |
| NOTCH1 | Functions as a receptor for membrane-bound ligands | 1.5 | 0.04 |
| | Jagged1, Jagged2 and Delta1 to regulate cell-fate | | |
| | determination. Upon ligand activation through the | | |
| | released notch intracellular domain (NICD) it forms a | | |
| | transcriptional activator complex with RBP-J kappa and | | |
| | activates genes of the enhancer of split locus. Affects the | | |
| | implementation of differentiation, proliferation and | | |
| | apoptotic programs. May be important for normal | | |
| | lymphocyte function. Involved in the maturation of both | | |
| | CD4+ and CD8+ cells in the thymus | | |
| CHN2 | GTPase-activating protein for p21-rac. Insufficient | 1.5 | 0.03 |
| | expression of beta-2 chimaerin is expected to lead to | | |
| | higher Rac activity and could therefore play a role in the | | |
| | progression from low-grade to high-grade tumors | | |
| SS18 | Synovial sarcoma, translocated to X chromosome | 1.5 | 0.02 |
| | (SSXT, SYT) | | |
| TEP1 | ATP/GTP-binding site motif A; G-protein beta WD-40 | 1.5 | 0.04 |
| | repeat | | |
| Q86XK7 | Immunoglobulin-like; Myelin P0 protein | 1.5 | 0.03 |
| LPAL2 | Chymotrypsin serine protease, family S1; Kringle; | 1.5 | 0.04 |
| | Prothrombin; Serine protease, trypsin family | | |
| DHRS3 | ATP/GTP-binding site motif A (P-loop); Glucose/ribitol | 1.5 | 0.03 |
| | dehydrogenase; Insect alcohol dehydrogenase family; | | |
| | Short-chain dehydrogenase/reductase SDR | | |
| PLAC1 | Acc: NM_021796]; placenta-specific 1. [Source: RefSeq | 1.5 | 0.01 |
| Q96SV6; | ATP/GTP-binding site motif A (P-loop); Bipartite | 1.5 | 0.05 |
| Q9UJY0 | nuclear localization signal; GTP-binding protein, HSR1- | | |
| | related | | |
| RFX1 | Regulatory factor essential for MHC class II genes | 1.5 | 0.06 |
| | expression. Binds to the X boxes of MHC class II genes. | | |
| | Also binds to an inverted repeat (ENH1) and to the most | | |
| | upstream element (alpha) of the RPL30 promoter | | |
| SACS | May function in chaperone-mediated protein folding | 1.5 | 0.01 |
| FRMD4 | Band 4.1 domain; Ezrin/radixin/moesin ERM | 1.5 | 0.04 |
| CCDC7 | Coiled-coil domain containing 7. | 1.5 | 0.05 |
| SKP2 | Substrate recognition component of the SCF (SKP1- | 1.5 | 0.03 |
| | CUL1-F-box protein) E3 ubiquitin ligase complex which | | |
| | mediates the ubiquitination and subsequent proteasomal | | |
| | degradation of target proteins involved in cell cycle | | |
| | progression, signal transduction and transcription. | | |
| | Specifically recognizes phosphorylated | | |
| | CDKN1B/p27kip and is involved in regulation of G1/S | | |
| | transition. | | |
| PCDHGC3 | Potential calcium-dependent cell-adhesion protein. May | 1.5 | 0.06 |
| | be involved in the establishment and maintenance of | | |
| | specific neuronal connections in the brain | | |
| WIF1 | Binds to WNT proteins and inhibits their activities. May | 1.5 | 0.01 |
| | be involved in mesoderm segmentation | | |
| RFC4 | The elongation of primed DNA templates by DNA | 1.5 | 0.04 |
| | polymerase delta and epsilon requires the action of the | | |
| | accessory proteins proliferating cell nuclear antigen | | |
| | (PCNA) and activator 1. The 37 kDa subunit may be | | |
| | involved in the elongation of the multiprimed DNA | | |
| | template | | |
| PFKFB3 | Synthesis and degradation of fructose 2,6-bisphosphate | 1.5 | 0.00 |
| RGS1 | Inhibits signal transduction by increasing the GTPase | 1.5 | 0.03 |
| | activity of G protein alpha subunits thereby driving them | | |
| | into their inactive GDP-bound form. This protein may be | | |
| | involved in the regulation of B-cell activation and | | |
| | proliferation | | |
| ZNF219 | May function as a transcription factor | 1.5 | 0.02 |
| DAAM1 | Binds to disheveled (Dvl) and Rho, and mediates Wnt- | 1.5 | 0.00 |
| | induced Dvl-Rho complex formation. May play a role as | | |
| | a scaffolding protein to recruit Rho-GDP and Rho-GEF, | | |
| | thereby enhancing Rho-GTP formation | | |
| UBE2G2 | Catalyzes the covalent attachment of ubiquitin to other | 1.5 | 0.04 |
| | proteins | | |
| PPP1R15B | Protein phosphatase 1, regulatory subunit 15B. | 1.5 | 0.02 |
| PLEKHA3 | Pleckstrin-like | 1.5 | 0.06 |
| GJA5 | One gap junction consists of a cluster of closely packed | 1.5 | 0.00 |
| | pairs of transmembrane channels, the connexons, | | |
| | through which materials of low MW diffuse from cell to | | |
| | neighboring cell | | |
| CACNA2D3 | Cache domain; von Willebrand factor, type A | 1.5 | 0.06 |
| SDK1 | Fibronectin, type III; Immunoglobulin-like | 1.5 | 0.06 |
| EIF2C2 | Provides endonuclease activity to RNA-induced | 1.5 | 0.04 |
| | silencing complexes (RISC). Cleaves siRNA/mRNA | | |
| | heteroduplexes bound to RISC. Essential for embryonic | | |
| | development as well as RNA-mediated gene silencing | | |
| | (RNAi) | | |
| ARVCF | Involved in protein-protein interactions at adherens | 1.5 | 0.06 |
| | junctions | | |
| HTLF | Binds to the purine-rich region in HTLV-I LTR | 1.5 | 0.02 |
| CD83 | May play a significant role in antigen presentation or the | 1.5 | 0.01 |
| | cellular interactions that follow lymphocyte activation | | |
| CST3 | As an inhibitor of cysteine proteinases, this protein is | 1.5 | 0.05 |
| | thought to serve an important physiological role as a | | |
| | local regulator of this enzyme activity | | |
| PAPOLB | Polymerase that creates the 3′ poly(A) tail of mRNA's. | 1.5 | 0.05 |
| | Also required for the endoribonucleolytic cleavage | | |
| | reaction at some polyadenylylation sites. May acquire | | |
| | specificity through interaction with a cleavage and | | |
| | polyadenylation specificity factor (CPSF) at its C- | | |
| | terminus | | |
| BIRC3 | Apoptotic suppressor. The BIR motifs region interacts | 1.5 | 0.02 |
| | with TNF receptor associated factors 1 and 2 (TRAF1 | | |
| | and TRAF2) to form an heteromeric complex, which is | | |
| | then recruited to the tumor necrosis factor receptor 2 | | |
| | (TNFR2) | | |
| NPC1L1 | Patched family | 1.5 | 0.02 |
| SERPINA1 | Inhibitor of serine proteases. Its primary target is | 1.5 | 0.05 |
| | elastase, but it also has a moderate affinity for plasmin | | |
| | and thrombin | | |
| MATN4 | Aspartic acid and asparagine hydroxylation site; EGF- | 1.5 | 0.06 |
| | like calcium-binding; EGF-like domain; von Willebrand | | |
| | factor, type A | | |
| Q9NWQ8 | Phosphoprotein associated with glycosphingolipid- | 1.5 | 0.03 |
| | enriched microdomains. | | |
| KCNJ2 | Inward rectifier potassium channels are characterized by | 1.5 | 0.01 |
| | a greater tendancy to allow potassium to flow into the | | |
| | cell rather than out of it. Probably participates in | | |
| | establishing action potential waveform and excitability | | |
| | of neuronal/muscle tissues. | | |
| LATS2 | Protein kinase; Serine/Threonine protein kinase; | 1.5 | 0.05 |
| | Ubiquitin-associated domain | | |
| DHX16 | Probable ATP-binding RNA helicase involved in pre- | 1.5 | 0.05 |
| | mRNA splicing | | |
| TIGD4 | CENP-B protein; CENP-B, N-terminal DNA-binding | 1.5 | 0.06 |
| CBX5 | Component of heterochromatin. Recognizes and binds | 1.5 | 0.05 |
| | histone H3 tails methylated at Lys-9, leading to | | |
| | epigenetic repression. May interact with lamin B | | |
| | receptor (LBR). | | |
| Q8TF25 | Ankyrin; Sterile alpha motif SAM | 1.5 | 0.02 |
| KIAA0355 | Hypothetical protein KIAA0355 | 1.5 | 0.00 |
| SLC11A1 | Divalent transition metal (iron and manganese) | 1.5 | 0.02 |
| | transporter involved in iron metabolism and host | | |
| | resistance to certain pathogens. | | |
| CUL5 | Component of E3 ubiquitin ligase complexes, which | −1.5 | 0.04 |
| | mediate the ubiquitination and subsequent proteasomal | | |
| | degradation of target proteins. May form a cell surface | | |
| | vasopressin receptor | | |
| IL6R | Part of the receptor for interleukin 6. Binds to IL-6 with | −1.5 | 0.03 |
| | low affinity, but does not transduce a signal. Signal | | |
| | activation necessitate an association with IL6ST. | | |
| | Activation may lead to the regulation of the immune | | |
| | response, acute-phase reactions and hematopoiesis | | |
| LY96 | Cooperates with TLR4 in the innate immune response to | −1.5 | 0.03 |
| | bacterial lipopolysaccharide (LPS), and with TLR2 in the | | |
| | response to cell wall components from Gram-positive | | |
| | and Gram-negative bacteria. Enhances TLR4-dependent | | |
| | activation of NFκB. Cells expressing both MD2 and | | |
| | TLR4, but not TLR4 alone, respond to LPS | | |
| ENSG00000159797 | low complexity | −1.5 | 0.03 |
| ACVR1 | On ligand binding, forms a receptor complex consisting | −1.5 | 0.02 |
| | of two type II and two type I transmembrane | | |
| | serine/threonine kinases. Type II receptors phosphorylate | | |
| | and activate type I receptors which autophosphorylate, | | |
| | then bind and activate SMAD transcriptional regulators. | | |
| | Receptor for activin | | |
| RNF8 | Probable E3 ubiquitin-protein ligase required to | −1.5 | 0.05 |
| | ubiquitinate some nuclear proteins, and promote their | | |
| | subsequent degradation | | |
| UACA | Ankyrin; Viral A-type inclusion protein repeat | −1.5 | 0.05 |
| Q8WWN1 | Protein kinase; SH3 domain; Serine/Threonine protein | −1.5 | 0.00 |
| | kinase; Tyrosine protein kinase | | |
| Q8TF72; | Bipartite nuclear localization signal; PDZ/DHR/GLGF | −1.5 | 0.04 |
| Q96K23 | domain; Proline-rich region | | |
| Q9BSW2 | Calcium-binding EF-hand | −1.5 | 0.03 |
| Q96L16 | LOC200420 | −1.5 | 0.01 |
| TRIM26 | Tripartite motif protein 26; Zinc finger protein 173; Acid | −1.5 | 0.00 |
| | finger protein; RING finger protein 95 | | |
| WBP1 | WW domain-binding protein 1; WBP-1 | −1.5 | 0.02 |
| CUL7 | Component of a probable SCF-like E3 ubiquitin ligase | −1.5 | 0.00 |
| | complex, which mediates the ubiquitination and | | |
| | subsequent proteosomal degaradation of target proteins. | | |
| | Probably plays a role in the degradation of proteins | | |
| | involved in endothelial proliferation and/or | | |
| | differentiation | | |
| MCCC2 | Methylcrotonoyl-CoA carboxylase beta chain, | −1.5 | 0.02 |
| | mitochondrial precursor | | |
| Q9Y5L9 | HMG-I and HMG-Y DNA-binding domain (A + T-hook); | −1.5 | 0.05 |
| | Helicase, C-terminal; Proline-rich extensin; SNF2 related | | |
| | domain | | |
| Q96GC0 | Splicing factor 3b, subunit 3, 130 kD. | −1.5 | 0.02 |
| FADD | Apoptotic adaptor molecule that recruits caspase-8 or | −1.5 | 0.05 |
| | caspase-10 to the activated Fas (CD95) or TNFR-1 | | |
| | receptors. The resulting aggregate called the death- | | |
| | inducing signaling complex (DISC) performs caspase-8 | | |
| | proteolytic activation. Active caspase-8 initiates the | | |
| | subsequent cascade of caspases (aspartate-specific | | |
| | cysteine proteases) mediating apoptosis | | |
| MPP7 | Guanylate kinase; L27 domain; PDZ/DHR/GLGF | −1.5 | 0.02 |
| | domain; SH3 domain | | |
| Q8N371 | Transcription factor jumonji, jmjC | −1.5 | 0.06 |
| UBE2R2 | Ubiquitin-conjugating enzymes | −1.5 | 0.04 |
| PGLYRP1 | Binds specifically to peptidoglycan and is involved in | −1.5 | 0.04 |
| | innate immunity | | |
| Q96CR0 | centrosome protein Cep63 | −1.5 | 0.04 |
| Q7Z3P6 | G-protein beta WD-40 repeat; Proline-rich region | −1.5 | 0.02 |
| Q9H8Y6 | Bipartite nuclear localization signal | −1.5 | 0.03 |
| C21orf33 | ES1 protein homolog, mitochondrial precursor; KNP-I; | −1.5 | 0.01 |
| | GT335 | | |
| SEC11L1 | Part of signal peptidase complex, exact function | −1.5 | 0.04 |
| | unknown | | |
| Q8N5H3 | Mouse Mammary Turmor Virus Receptor homolog 1. | −1.5 | 0.02 |
| FER | Non-receptor tyrosine kinase. Probably performs | −1.5 | 0.00 |
| | important function, in regulatory processes such as cell | | |
| | cycle control | | |
| Q8IUY5 | Actin; Actin/actin-like | −1.5 | 0.02 |
| Q96H61 | low complexity | −1.5 | 0.02 |
| C6orf208 | unknown | −1.5 | 0.04 |
| COL11A2 | May play an important role in fibrillogenesis by | −1.5 | 0.05 |
| | controlling lateral growth of collagen II fibrils | | |
| PRRG1 | Transmembrane proline-rich γ-carboxyglutamic acid | −1.5 | 0.04 |
| | protein 1 | | |
| PPAP2A | PA-phosphatase related phosphoesterase | −1.5 | 0.01 |
| TIMP2 | Complexes with metalloproteinases (such as | −1.5 | 0.03 |
| | collagenases) and irreversibly inactivates them. Known | | |
| | to act on MMP-1, MMP-2, MMP-3, MMP-7, MMP-8, | | |
| | MMP-9, MMP-10, MMP-13, MMP-14, MMP-15, | | |
| | MMP-16 and MMP-19 | | |
| FHOD1 | Required for the assembly of F-actin structures, such as | −1.5 | 0.01 |
| | stress fibers. Depends on the Rho-ROCK cascade for its | | |
| | activity. Contributes to the coordination of microtubules | | |
| | with actin fibers and plays a role in cell elongation | | |
| NOTCH1 | Functions as a receptor for membrane-bound ligands | −1.5 | 0.05 |
| | Jagged1, Jagged2 and Delta1 to regulate cell-fate | | |
| | determination. Upon ligand activation through the | | |
| | released notch intracellular domain (NICD) it forms a | | |
| | transcriptional activator complex with RBP-Jκ and | | |
| | activates genes of the enhancer of split locus. Affects the | | |
| | implementation of differentiation, proliferation and | | |
| | apoptotic programs. May be important for normal | | |
| | lymphocyte function. In altered form, may contribute to | | |
| | transformation or progression in some T-cell neoplasms. | | |
| | Involved in the maturation of both CD4+ and CD8+ cells | | |
| | in the thymus. | | |
| HINT3 | Histidine triad (HIT) protein | −1.5 | 0.04 |
| POLR2L | DNA-dependent RNA polymerase | −1.5 | 0.03 |
| ZNF354A | Transcription factor zinc finger protein 354A, 17, eZNF | −1.5 | 0.05 |
| INPP5D | Endonuclease/exonuclease/phosphatase family; Proline- | −1.5 | 0.00 |
| | rich extensin; SH2 motif | | |
| C9orf37 | low complexity | −1.5 | 0.02 |
| HPGD | Inactivation of prostaglandins | −1.5 | 0.05 |
| Q8WYL1 | Dual specificity protein phosphatase | −1.5 | 0.01 |
| GBX1 | Homeobox protein GBX-1; Gastrulation and brain- | −1.5 | 0.05 |
| | specific homeobox protein 1 | | |
| ZCCHC4 | ATP/GTP-binding site motif A (P-loop); Bipartite | −1.6 | 0.04 |
| | nuclear localization signal; N-6 Adenine-specific DNA | | |
| | methylase; Zn-finger, CCHC type; Zn-finger, DHHC | | |
| | type | | |
| Q8N3J9 | Zn-finger, C2H2 subtype; | −1.6 | 0.00 |
| CBX1 | Component of heterochromatin. Recognizes and binds | −1.6 | 0.04 |
| | histone H3 tails methylated at Lys-9, leading to | | |
| | epigenetic repression. May interact with lamin B | | |
| | receptor (LBR). | | |
| PARD3 | Adapter protein involved in asymmetrical cell division | −1.6 | 0.05 |
| | and cell polarization processes. Seems to play a central | | |
| | role in the formation of epithelial tight junctions. | | |
| SERPINA10 | Inhibits factor Xa activity in the presence of protein Z, | −1.6 | 0.05 |
| | calcium and phospholipid | | |
| CENTG3 | GTPase-activating protein for the ADP ribosylation | −1.6 | 0.06 |
| | factor family | | |
| ARHGEF19 | Bipartite nuclear localization signal; DH domain; | −1.6 | 0.05 |
| | Pleckstrin-like; SH3 domain | | |
| Q8IWC2 | ATP/GTP-binding site motif A (P-loop); Proline-rich | −1.6 | 0.02 |
| | extensin | | |
| TCP10 | T-complex protein 10A homolog | −1.6 | 0.04 |
| ENSG00000170714 | low complexity | −1.6 | 0.04 |
| TPCN1 | Ion transport protein; Legume lectin, beta domain | −1.6 | 0.03 |
| APP | Functions as a cell surface receptor. Involved in cell | −1.6 | 0.05 |
| | mobility and transcription regulation through protein- | | |
| | protein interactions. Can promote transcription activation | | |
| | through binding to APBB1/Tip60 and inhibit Notch | | |
| | signaling through interaction with Numb. Couples to | | |
| | apoptosis-inducing pathways such as those mediated by | | |
| | G(O) and JIP. | | |
| IDI1 | Catalyzes the 1,3-allylic rearrangement of the | −1.6 | 0.00 |
| | homoallylic substrate isopentenyl (IPP) to its highly | | |
| | electrophilic allylic isomer, dimethylallyl diphosphate | | |
| | (DMAPP) | | |
| EZH1 | May be involved in the regulation of gene transcription | −1.6 | 0.06 |
| | and chromatin structure | | |
| ASB7 | Ankyrin repeat and SOCS box protein 7; ASB-7 | −1.6 | 0.04 |
| ACTL7A | Actin-like protein 7A; Actin-like-7-alpha; Actin-like 7A | −1.6 | 0.00 |
| GAPD | Glyceraldehyde-3-phosphate dehydrogenase; GAPDH | −1.6 | 0.01 |
| SULT1E1 | May control the level of the estrogen receptor by | −1.6 | 0.01 |
| | sulfurylating free estradiols etc. | | |
| Q9BV79 | Zinc-containing alcohol dehydrogenase superfamily | −1.6 | 0.05 |
| C6orf110 | Protein of unknown function DUF221 | −1.6 | 0.03 |
| Q8IXK7 | low complexity; transmembrane | −1.6 | 0.02 |
| NRCAM | Cell adhesion, ankyrin-binding protein involved in | −1.6 | 0.05 |
| | neuron-neuron adhesion. | | |
| ACTA2 | Actins are highly conserved proteins that are involved in | −1.6 | 0.04 |
| | various types of cell motility and are ubiquitously | | |
| | expressed in all eukaryotic cells | | |
| Q9BPX8 | unknown | −1.6 | 0.02 |
| Q9HCK1 | low complexity | −1.6 | 0.00 |
| STK29 | BR serine/threonine-protein kinase 2, 29; SAD1B | −1.6 | 0.01 |
| MAGEA9 | May play a role in embryonal development and tumor | −1.6 | 0.05 |
| | transformation or aspects of tumor progression | | |
| GCAT | 2-amino-3-ketobutyrate coenzyme A ligase, | −1.6 | 0.01 |
| | mitochondrial | | |
| NPAS2 | Neuronal PAS domain protein 2; Member of PAS | −1.6 | 0.00 |
| | protein 4; MOP4 | | |
| GLRX | Has a glutathione-disulfide oxidoreductase activity in the | −1.6 | 0.04 |
| | presence of NADPH and glutathione reductase. | | |
| CLECSF6 | C-type lectin; Type II antifreeze protein | −1.6 | 0.02 |
| OSBPL11 | Oxysterol binding protein-related protein 11 | −1.6 | 0.00 |
| ANKRD10 | Ankyrin repeat domain protein 10 | −1.6 | 0.04 |
| SH3MD3 | SH3 domain | −1.6 | 0.01 |
| SFRP5 | Bipartite nuclear localization signal; Frizzled CRD | −1.6 | 0.05 |
| | region; Netrin, C-terminal | | |
| Q8IZW8 | SH2 motif | −1.6 | 0.02 |
| OVOL1 | Putative transcription factor. Involved in hair formation | −1.6 | 0.02 |
| | and spermatogenesis. May function in the differentiation | | |
| | and/or maintenance of the urogenital system | | |
| INSR | This receptor binds insulin and has a tyrosine-protein | −1.6 | 0.01 |
| | kinase activity. Isoform Short has a higher affinity for | | |
| | insulin | | |
| RBPSUHL | Putative transcription factor, which cooperates with | −1.6 | 0.03 |
| | EBNA2 to activate transcription | | |
| PRM2 | Protainines substitute for histones in the chromatin of | −1.6 | 0.02 |
| | sperm during the haploid phase of spermatogenesis & | | |
| | compact sperm DNA into a highly condensed, stable and | | |
| | inactive complex | | |
| PARVA | Probably plays a role in the regulation of cell adhesion | −1.6 | 0.03 |
| | and cytoskeleton organization | | |
| VPS4B | Involved in intracellular protein transport probably out of | −1.6 | 0.04 |
| | a prevacuolar endosomal compartment. May be involved | | |
| | in the release of components of the bilayered coat from | | |
| | the endosomal membrane. In case of infection, the HIV- | | |
| | 1 virus takes advantage of it for budding and exocytic | | |
| | cargoes of viral proteins | | |
| APRIN | HMG-I and HMG-Y DNA-binding domain (A + T-hook) | −1.6 | 0.04 |
| Q96MI8 | ATP/GTP-binding site motif A (P-loop) | −1.6 | 0.06 |
| CDH8 | Cadherins are calcium dependent cell adhesion proteins. | −1.6 | 0.04 |
| | They preferentially interact with themselves in a | | |
| | homophilic manner in connecting cells; cadherins may | | |
| | thus contribute to the sorting of heterogeneous cell types | | |
| PIK3C2B | Phosphorylates PtdIns and PtdIns4P with a preference | −1.6 | 0.05 |
| | for PtdIns. Does not phosphorylate PtdIns(4,5)P2. May | | |
| | be involved in EGF and PDGF signaling cascades | | |
| CALD1 | Actin- and myosin-binding protein implicated in the | −1.6 | 0.05 |
| | regulation of actomyosin interactions in smooth muscle | | |
| | and nonmuscle cells (could act as a bridge between | | |
| | myosin and actin filaments). Also plays an essential role | | |
| | during cellular mitosis and receptor capping. | | |
| ARRDC1 | Arrestin | −1.6 | 0.01 |
| Q9BWC9 | Bipartite nuclear localization signal | −1.6 | 0.01 |
| MC4R | Receptor specific to the heptapeptide core common to | −1.6 | 0.02 |
| | adrenocorticotropic hormone and alpha-, beta-, and | | |
| | gamma-MSH. This receptor is mediated by G proteins | | |
| | that stimulate adenylate cyclase | | |
| ELMO1 | Involved in cytoskeletal rearrangements required for | −1.6 | 0.03 |
| | phagocytosis of apoptotic cells and cell motility. Acts in | | |
| | assocation with DOCK1 and CRK. Was initially | | |
| | proposed to be required in complex with DOCK1 to | | |
| | activate Rac Rho small GTPases. May enhance the | | |
| | guanine nucleotide exchange factor (GEF) activity of | | |
| | DOCK1 | | |
| MPDZ | PDZ/DHR/GLGF domain | −1.6 | 0.06 |
| ENSG00000079548 | low complexity | −1.6 | 0.06 |
| FECH | Catalyzes the ferrous insertion into protoporphyrin IX | −1.6 | 0.01 |
| PNMA2 | Paraneoplastic antigen Ma2 | −1.6 | 0.04 |
| KCNMB4 | Calcium-activated BK potassium channel, beta subunit | −1.6 | 0.04 |
| STAG1 | Component of cohesin complex, a complex required for | −1.6 | 0.05 |
| | the cohesion of sister chromatids after DNA replication. | | |
| MPPE1 | Hemopexin repeat; Metallo-phosphoesterase | −1.6 | 0.02 |
| Q8N6S2 | Immunoglobulin-like; N-6 Adenine-specific DNA | −1.6 | 0.06 |
| | methylase | | |
| UBR1 | Polyprenyl synthetase; Ribosomal protein S16; Zn-finger | −1.6 | 0.01 |
| | (putative), N-recognin | | |
| WASF2 | Actin-binding WH2; Bipartite nuclear localization | −1.6 | 0.02 |
| | signal; Histamine H3 receptor; Pistil-specific extensin- | | |
| | like protein; Proline-rich extensin | | |
| ABR | GTPase-activating protein for RAC and CDC42. | −1.6 | 0.01 |
| | Promotes the exchange of RAC or CDC42-bound GDP | | |
| | by GTP, thereby activating them | | |
| CYP1B1 | Cytochromes P450 are a group of heme-thiolate | −1.6 | 0.02 |
| | monooxygenases. In liver microsomes, this enzyme is | | |
| | involved in an NADPH-dependent electron transport | | |
| | pathway. It oxidizes a variety of structurally unrelated | | |
| | compounds, including steroids, fatty acids, and | | |
| | xenobiotics | | |
| EEF2K | Phosphorylates eukaryotic elongation factor-2. Binds | −1.6 | 0.03 |
| | calmodulin | | |
| HIST1H2BN | Histone H2B | −1.6 | 0.00 |
| BRD3 | Bromodomain-containing protein 3; RING3-like protein | −1.6 | 0.04 |
| MFN1 | ATP/GTP-binding site motif A (P-loop); Fzo-like | −1.6 | 0.05 |
| | conserved region | | |
| C11orf21 | Protein C11orf21 | −1.6 | 0.00 |
| ZFP64 | May function as a transcription factor | −1.6 | 0.02 |
| PLA2G5 | PA2 catalyzes the calcium-dependent hydrolysis of the | −1.6 | 0.02 |
| | 2-acyl groups in 3-sn-phosphoglycerides. May be | | |
| | involved in the production of lung surfactant, the | | |
| | remodeling or regulation of cardiac muscle | | |
| Q96HP4 | Flavoprotein pyridine nucleotide cytochrome reductase; | −1.6 | 0.00 |
| | NADH: cytochrome b5 reductase (CBR); Oxidoreductase | | |
| | FAD/NAD(P)-binding; Phenol hydroxylase reductase | | |
| MYOZ2 | FATZ related protein 2; calcineurin-binding protein | −1.6 | 0.04 |
| | calsarcin-1 | | |
| GOLGB1 | May participate in forming intercisternal cross-bridges of | −1.6 | 0.04 |
| | the Golgi complex | | |
| Q96HQ2 | low complexity | −1.6 | 0.06 |
| TESK2 | Dual specificity protein kinase activity catalyzing | −1.6 | 0.02 |
| | autophosphorylation and phosphorylation of exogenous | | |
| | substrates on both serine/threonine and tyrosine residues. | | |
| | Phosphorylates cofilin at Ser-3. | | |
| AK2 | This small ubiquitous enzyme is essential for | −1.6 | 0.01 |
| | maintenance and cell growth | | |
| Q9C0D3 | Bipartite nuclear localization signal; Leucine-rich repeat | −1.6 | 0.03 |
| ZNF347 | Bipartite nuclear localization signal; KRAB box; Zn- | −1.6 | 0.05 |
| | finger, C2H2 subtype | | |
| MUC20 | ATP/GTP-binding site motif A (P-loop) | −1.7 | 0.04 |
| CLECSF14 | C-type lectin; Type II antifreeze protein | −1.7 | 0.01 |
| ECM1 | Extracellular matrix protein 1 precursor; Secretory | −1.7 | 0.03 |
| | component p85 | | |
| Q9P021 | HSPC139 protein; postsynaptic protein CRIPT | −1.7 | 0.01 |
| Q9P194 | unknown | −1.7 | 0.04 |
| Q96CD2 | Flavoprotein | −1.7 | 0.02 |
| STMN4 | Stathmin-4; Stathmin-like protein B3; RB3 | −1.7 | 0.06 |
| APTX | Histidine triad (HIT) protein; Zn-finger, C2H2 type. | −1.7 | 0.05 |
| Q8IYX7 | unknown | −1.7 | 0.01 |
| CXXC5 | Bipartite nuclear localization signal; Zn-finger, CXXC | −1.7 | 0.02 |
| | type | | |
| PPM1A | Enzyme with a broad specificity | −1.7 | 0.02 |
| Q9P2I9 | Bipartite nuclear localization signal | −1.7 | 0.04 |
| Q9BSD4 | coiled-coil; low complexity | −1.7 | 0.05 |
| THAP9 | Bipartite nuclear localization signal | −1.7 | 0.05 |
| PLXDC2 | Plexin | −1.7 | 0.01 |
| Q9NTK9 | DJ1092A11.1 (Hypothetical protein KIAA0495). | −1.7 | 0.04 |
| MTIF2 | One of the essential components for the initiation of | −1.7 | 0.00 |
| | protein synthesis. | | |
| RNF122 | Zn-finger, RING | −1.7 | 0.04 |
| CRYZL1 | Quinone oxidoreductase-like 1; Zeta-crystallin homolog; | −1.7 | 0.04 |
| | 4P11 | | |
| SLC39A10 | Cytochrome c heme-binding site; Zinc transporter ZIP | −1.7 | 0.03 |
| AKAP11 | Binds to the N-terminal PTS2-type peroxisomal targeting | −1.7 | 0.02 |
| | signal and plays an essential role in peroxisomal protein | | |
| | import; Binds to type II regulatory subunits of protein | | |
| | kinase A and anchors/targets them to the membrane. | | |
| | May anchor the kinase to cytoskeletal and/or organelle- | | |
| | associated proteins; Ligand of the T lymphocyte CD2 | | |
| | glycoprotein. | | |
| TNNC2 | Troponin is the central regulatory protein of striated | −1.7 | 0.02 |
| | muscle contraction. Tn consists of three components: Tn- | | |
| | I which is the inhibitor of actomyosin ATPase, Tn-T | | |
| | which contains the binding site for tropomyosin and Tn- | | |
| | C. The binding of calcium to Tn-C abolishes the | | |
| | inhibitory action of Tn on actin filaments | | |
| TTC17 | TPR repeat | −1.7 | 0.01 |
| NPTX1 | May mediate uptake of degraded synaptic material which | −1.7 | 0.01 |
| | could play an important role in synaptic remodeling. | | |
| FMNL2 | Actin-binding FH2; Proline-rich extensin; Wilm's | −1.7 | 0.01 |
| | tumour protein | | |
| HLA-DMA | Plays a critical role in catalyzing the release of class II | −1.7 | 0.01 |
| | HLA-associated invariant chain-derived peptides (CLIP) | | |
| | from newly synthesized class II HLA molecules and | | |
| | freeing the peptide binding site for acquisition of | | |
| | antigenic peptides | | |
| SLC22A13 | General substrate transporter | −1.7 | 0.05 |
| H2AFZ | Variant histones H2A are synthesized throughout the cell | −1.7 | 0.00 |
| | cycle and are very different from classical S-phase | | |
| | regulated H2A. The exact function of variant histones | | |
| | H2A is not known | | |
| HNRPM | Pre-mRNA binding protein in vivo, binds avidly to | −1.7 | 0.01 |
| | poly(G) and poly(U) RNA homopolymers in vitro. | | |
| | Involved in splicing. Acts as a receptor for | | |
| | carcinoembryonic antigen in Kupffer cells, may initiate a | | |
| | series of signaling events leading to tyrosine | | |
| | phosphorylation of proteins and induction of IL-1 alpha, | | |
| | IL-6, IL-10 and tumor necrosis factor alpha cytokines | | |
| OR5A2 | Putative odorant receptor | −1.7 | 0.04 |
| SV2B | General substrate transporter; Sugar transporter | −1.7 | 0.00 |
| | superfamily | | |
| DNTTIP1 | Shown to enhance TdT activity, in vitro | −1.7 | 0.04 |
| HSPA1L | In cooperation with other chaperones, Hsp70s stabilize | −1.7 | 0.02 |
| | preexistent proteins against aggregation and mediate the | | |
| | folding of newly translated polypeptides in the cytosol as | | |
| | well as within organelles. | | |
| KIAA0258 | Protein KIAA0258 | −1.7 | 0.02 |
| MRPS16 | 28S ribosomal protein S16, mitochondrial precursor | −1.7 | 0.04 |
| ITGA10 | Integrin alpha-10/beta-1 is a receptor for collagen | −1.7 | 0.05 |
| IL10RB | Receptor for IL10 and IL22. Serves as an accessory | −1.7 | 0.02 |
| | chain essential for the active IL10 receptor complex and | | |
| | to initiate IL10-induced signal transduction events | | |
| Q9C0D5 | ATP/GTP-binding site motif A (P-loop); Ankyrin; TPR | −1.7 | 0.04 |
| | repeat | | |
| C21orf57 | UPF0054 protein C21orf57 | −1.7 | 0.03 |
| TMH | unknown | −1.7 | 0.05 |
| PLA2G4C | Lysophospholipase, catalytic domain; Prenyl group | −1.7 | 0.01 |
| | binding site (CAAX box) | | |
| PPAT | Glutamine phosphoribosylpyrophosphate | −1.7 | 0.01 |
| | amidotransferase | | |
| Q7Z570 | Zn-finger, C2H2 matrin type | −1.7 | 0.06 |
| ARL3 | Does not act as an allosteric activator of the cholera toxin | −1.7 | 0.02 |
| | catalytic subunit | | |
| PHF5A | Acts as a transcriptional regulator by binding to the | −1.7 | 0.04 |
| | GJA1/Cx43 promoter and enhancing its up-regulation by | | |
| | ESR1/ER-alpha. Also involved in pre-mRNA splicing | | |
| MTHFR | Catalyzes the conversion of 5,10- | −1.7 | 0.02 |
| | methylenetetrahydrofolate to 5-methyltetrahydrofolate, a | | |
| | co-substrate for homocysteine remethylation to | | |
| | methionine | | |
| MARK4 | MAP/microtubule affinity-regulating kinase 4 | −1.8 | 0.05 |
| MVP | Unknown, though MVP is required for normal vault | −1.8 | 0.05 |
| | structure. Vaults are multi-subunit structures that may be | | |
| | involved in nucleo-cytoplasmic transport | | |
| USP6NL | RabGAP/TBC domain | −1.8 | 0.02 |
| PAPPA | Metalloproteinase which specifically cleaves IGFBP-4 | −1.8 | 0.04 |
| | and IGFBP-5, releasing bound IGF. Cleavage of IGFBP- | | |
| | 4 is dramatically enhanced by IGF, whereas cleavage of | | |
| | IGFBP-5 is slightly inhibited by the presence of IGF | | |
| RAB27B | Ras-related protein Rab-27B; C25KG | −1.8 | 0.02 |
| BNIP3L | Induces apoptosis. Interacts with viral and cellular anti- | −1.8 | 0.05 |
| | apoptosis proteins. Can overcome the suppressers BCL-2 | | |
| | and BCL-XL, although high levels of BCL-XL | | |
| | expression will inhibit apoptosis. May function as a | | |
| | tumor suppressor | | |
| ENSG00000187712 | Actin/actin-like | −1.8 | 0.03 |
| C6orf80 | low complexity | −1.8 | 0.02 |
| Q96EG4 | low complexity | −1.8 | 0.06 |
| 095893 | transmembrane | −1.8 | 0.04 |
| COQ4 | Ubiquinone biosynthesis protein COQ4 homolog; | −1.8 | 0.01 |
| DDC | Catalyzes the decarboxylation of DOPA to dopamine, L- | −1.8 | 0.04 |
| | 5-hydroxytryptophan to serotonin and L-trp to | | |
| | tryptamine | | |
| ZNF431 | May function as a transcription factor | −1.8 | 0.03 |
| JAK2 | Tyrosine kinase of the non-receptor type, involved in | −1.8 | 0.03 |
| | interleukin 3 signal transduction | | |
| RNF34 | Cytochrome c heme-binding site; Zn-finger, RING | −1.8 | 0.02 |
| Q9BYA4 | unknown | −1.8 | 0.06 |
| ATP1B4 | This is the non-catalytic component of a yet unknown | −1.8 | 0.03 |
| | soduium or proton exchange ATPase | | |
| PCDHGC3 | Potential calcium-dependent cell-adhesion protein. | −1.8 | 0.03 |
| C6orf33 | Hly-III related proteins | −1.8 | 0.02 |
| Q96BY9 | NULL | −1.8 | 0.04 |
| Q9BZS9 | PNAS-138. | −1.8 | 0.03 |
| Q96K66 | signal peptide | −1.8 | 0.02 |
| Q9Y4M2 | low complexity | −1.9 | 0.02 |
| CDC14B | Tyrosine specific protein phosphatase; Tyrosine specific | −1.9 | 0.04 |
| | protein phosphatase and dual specificity protein | | |
| | phosphatase | | |
| CALM3 | Calmodulin mediates the control of a large number of | −1.9 | 0.05 |
| | enzymes by Ca(2+). Among the enzymes to be | | |
| | stimulated by the calmodulin-Ca(2+) complex are a | | |
| | number of protein kinases and phosphatases | | |
| SLC16A9 | Solute carrier family 16 (monocarboxylic acid | −1.9 | 0.05 |
| | transporters), member 9 | | |
| Q8TEB9 | Rhomboid-like protein; Ubiquitin interacting motif | −1.9 | 0.02 |
| Q9UPP5 | coiled-coil; low complexity | −1.9 | 0.01 |
| Q8IUT6 | low complexity; transmembrane | −1.9 | 0.01 |
| ARRB1 | Beta-arrestins seem to bind phosphorylated beta- | −1.9 | 0.03 |
| | adrenergic receptors and regulate function, thereby | | |
| | causing a significant impairment of their capacity to | | |
| | activate G(S) proteins | | |
| ATP6V1H | Subunit of the peripheral V1 complex of vacuolar | −1.9 | 0.00 |
| | ATPase. Subunit H activate ATPase activity of the | | |
| | enzyme and couple ATPase activity to proton flow. | | |
| | Vacuolar ATPase is responsible for acidifying a variety | | |
| | of intracellular compartments in eukaryotic cells. | | |
| | Involved in the endocytosis mediated by clathrin-coated | | |
| | pits, required for the formation of endosomes | | |
| Q969E4 | Bipartite nuclear localization signal | −1.9 | 0.02 |
| GBAS | Protein NipSnap2; Glioblastoma amplified sequence | −1.9 | 0.03 |
| MYCL1 | L-myc-1 proto-oncogene protein | −1.9 | 0.04 |
| C22orf3 | Protein C22orf3 | −1.9 | 0.00 |
| EPHX2 | Acts on epoxides (alkene oxides, oxiranes) and arene | −1.9 | 0.03 |
| | oxides. Also determines steady-state levels of | | |
| | physiological mediators. | | |
| ZFP36L2 | Probable regulatory protein involved in regulating the | −1.9 | 0.04 |
| | response to growth factors | | |
| Q9NS00 | Acc: NM_020156]; core1 UDP-galactose: N- | −1.9 | 0.03 |
| | acetylgalactosamine-alpha-R beta 1,3-galac. | | |
| | [Source:RefSeq | | |
| O95510 | ABC transporter; Acyl transferase domain | −1.9 | 0.00 |
| Q8TB55 | Proline-rich region | −1.9 | 0.00 |
| Q9Y3S6 | RhoGAP domain | −1.9 | 0.03 |
| FER | Tyrosine kinase of the non-receptor type. Probably | −1.9 | 0.05 |
| | performs an important function, perhaps in regulatory | | |
| | processes such as cell cycle control | | |
| Q8NAA4 | G-protein beta WD-40 repeat | −1.9 | 0.00 |
| ENSG00000115404 | Enoyl-CoA hydratase/isomerase | −2.0 | 0.04 |
| DHRS9 | Glucose/ribitol dehydrogenase; Short-chain | −2.0 | 0.05 |
| | dehydrogenase/reductase SDR | | |
| ELOVL5 | GNS1/SUR4 membrane protein | −2.0 | 0.01 |
| IL13RA1 | Binds IL13 with a low affinity. Together with IL4R- | −2.0 | 0.00 |
| | alpha can form a functional receptor for IL13. Also | | |
| | serves as an alternate accessory protein to the common | | |
| | cytokine receptor gamma chain for IL4 signaling, but | | |
| | cannot replace the function of gamma C in allowing | | |
| | enhanced IL2 binding activity | | |
| Q8IXM2 | low complexity | −2.0 | 0.05 |
| CYB5 | Cytochrome b5 is a membrane bound hemoprotein that | −2.0 | 0.01 |
| | functions as an electron carrier for several membrane | | |
| | bound oxygenases | | |
| MAP1B | Phosphorylated MAP1B may play a role in the | −2.0 | 0.04 |
| | cytoskeletal changes that accompany neurite extension. | | |
| | Possibly MAP1B Binds to at least two tubulin subunits | | |
| | in the polymer, and this bridging of subunits might be | | |
| | involved in nucleating microtubule polymerization and | | |
| | in stabilizing microtubules | | |
| CABP2 | Calcium-binding protein 2; CaBP2 | −2.0 | 0.01 |
| Q8TDG4 | DEAD/DEAH box helicase; Helicase, C-terminal | −2.0 | 0.05 |
| FAM31B | DENN (AEX-3) domain; Proline-rich extensin; dDENN | −2.0 | 0.04 |
| | domain | | |
| KIAA1244 | Essential component of the high affinity receptor for the | −2.0 | 0.04 |
| | general membrane fusion machinery and an important | | |
| | regulator of transport vesicle docking and fusion | | |
| CDK2AP1 | Cyclin-dependent kinase 2-associated protein 1; Putative | −2.0 | 0.06 |
| | oral cancer suppressor. | | |
| C5orf5 | Protein C5orf5; GAP-like protein N61 | −2.0 | 0.03 |
| MOCS3 | Activates MPT synthase by the ATP dependant | −2.1 | 0.05 |
| | adenylation of its C-terminal residue | | |
| UGT2B7 | Heavy metal transport/detoxification protein; UDP- | −2.1 | 0.01 |
| | glucoronosyl/UDP-glucosyl transferase | | |
| ARL7 | Binds and exchanges GTP and GDP | −2.1 | 0.00 |
| CD4 | Accessory protein for MHC class-II antigen/T-cell | −2.1 | 0.01 |
| | receptor interaction. May regulate T-cell activation | | |
| KRT7 | Keratin, type II cytoskeletal 7; Cytokeratin-7; | −2.1 | 0.05 |
| | Sarcolectin | | |
| ENSG00000142954 | 4Fe—4S ferredoxin, iron-sulfur binding domain; | −2.1 | 0.01 |
| | Dihydroorotate dehydrogenase; FMN/related compound- | | |
| | binding core | | |
| Q96FP9 | Calponin-like actin-binding; Leucine-rich repeat | −2.1 | 0.05 |
| GNAS | The G(s) Guanine nucleotide-binding protein is involved | −2.1 | 0.04 |
| | in hormonal regulation of adenylate cyclase: it activates | | |
| | the cyclase in response to beta-adrenergic stimuli | | |
| Q9P1V9 | low complexity | −2.1 | 0.01 |
| Q9Y547 | HSPCO34 protein. | −2.2 | 0.02 |
| ENSG00000177876 | Thrombospondin, type I | −2.2 | 0.02 |
| Q9BR68 | Ran-interacting Mog1 protein | −2.2 | 0.03 |
| CLGN | Probably plays an important role in spermatogenesis. | −2.2 | 0.01 |
| | Binds calcium ions | | |
| Q96FV0 | Leucine-rich repeat | −2.2 | 0.01 |
| PLAGL2 | Shows weak transcriptional activatory activity | −2.2 | 0.04 |
| ITM2B | Integral membrane protein 2B; Transmembrane protein | −2.2 | 0.06 |
| | BRI; ABri/ADan amyloid peptide | | |
| Q8IY68 | low complexity | −2.2 | 0.03 |
| SLC1A4 | Transporter for alanine, serine, cysteine, and threonine. | −2.3 | 0.05 |
| | Exhibits sodium dependence | | |
| TIGD7 | CENP-B protein; CENP-B, N-terminal DNA-binding | −2.3 | 0.02 |
| VTI1A | V-SNARE that mediates vesicle transport pathways | −2.3 | 0.06 |
| | through interactions with t-SNAREs on the target | | |
| | membrane. These interactions are proposed to mediate | | |
| | aspects of the specificity of vesicle trafficking and to | | |
| | promote fusion of the lipid bilayers. May be concerned | | |
| | with increased secretion of cytokines associated with | | |
| | cellular senescence | | |
| ITGBL1 | Integrin beta, C-terminal; Laminin-type EGF-like | −2.3 | 0.01 |
| | domain | | |
| Q9C093 | ATP/GTP-binding site motif A (P-loop) | −2.3 | 0.03 |
| FOS | Nuclear phosphoprotein which forms a tight but non- | −2.3 | 0.00 |
| | covalently linked complex with the JUN/AP-1 | | |
| | transcription factor. In the heterodimer, c-fos and | | |
| | JUN/AP-1 basic regions each seems to interact with | | |
| | symmetrical DNA half sites. C-fos has a critical function | | |
| | in regulating the development of cells destined to form | | |
| | and maintain the skeleton. it is thought to have an | | |
| | important role in signal transduction, cell proliferation | | |
| | and differentiation | | |
| LARS | Aminoacyl-tRNA synthetase, class Ia | −2.5 | 0.01 |
| Q96FR9 | Bipartite nuclear localization signal; Exonuclease | −2.5 | 0.01 |
| P2RY5 | P2Y purinoceptor 5; P2Y5; | −2.5 | 0.01 |
| SLC26A10 | DH domain | −2.5 | 0.02 |
| PHTF1 | May play a role in transcription regulation | −2.6 | 0.00 |
| DEFB127 | Has antibacterial activity | −2.8 | 0.00 |
| MEF2C | Transcription activator which binds specifically to the | −2.8 | 0.04 |
| | MEF2 element present in the regulatory regions of many | | |
| | muscle-specific genes | | |
| DBR1 | Lariat debranching enzyme, C-terminal; Metallo- | −2.8 | 0.05 |
| | phosphoesterase | | |
| DHRS8 | 2,3-dihydro-2,3-dihydroxybenzoate dehydrogenase; | −3.0 | 0.02 |
| | Short-chain dehydrogenase/reductase SDR | | |
| O75915 | Prenylated rab acceptor PRA1 | −3.3 | 0.03 |
| CD36 | Seems to have numerous potential physiological | −5.8 | 0.05 |
| | functions. Binds to collagen, thrombospondin, anionic | | |
| | phospholipids and oxidized LDL. May function as a cell | | |
| | adhesion molecule. Directly mediates cytoadherence of | | |
| | Plasmodium falciparum parasitized erythrocytes. Binds | | |
| | long chain fatty acids and may function in the transport | | |
| | and/or as a regulator of fatty acid transport | | |
| RBP1 | Intracellular transport of retinol | 125.8 | 0.05 |

TABLE 72
Thirty nine genes that were up-regulated by LPS and somewhat suppressed in
the presence of peptide SEQ ID NO: 7. Such genes reflect the anti-endotoxic activity of
SEQ ID NO: 7. The data reveal that the residual levels of expression can be largely
accounted for by the response to peptide SEQ ID NO: 7 alone.

| | | | | | | Fold | |
|---|---|---|---|---|---|---|---|
| | | Fold | p- | Fold | p- | Change | |
| Gene | | change | student | change | student | by | p-student |
| Name | Gene Description | by LPS | (LPS) | by LPS + peptide | (LPS + peptide) | peptide | (peptide) |
| GPD1 | Cytoplasmic glycerol-3-phosphate | 103.4 | 0.06 | 89.7 | 0.04 | 65.3 | 0.09 |
| | dehydrogenase [NAD+], | | | | | | |
| Q8NI35 | ATP/GTP-binding site motif A (P- | 78.4 | 0.06 | 53.9 | 0.06 | 57.2 | 0.05 |
| | loop); | | | | | | |
| FEZ2 | Involved in axonal outgrowth and | 45.9 | 0.05 | 25.8 | 0.02 | 40.2 | 0.02 |
| | fasciculation | | | | | | |
| NRXN1 | Neuronal cell surface protein that may | 35.4 | 0.05 | 25.9 | 0.04 | 31.9 | 0.09 |
| | be involved in cell recognition and cell | | | | | | |
| | adhesion. May mediate intracellular | | | | | | |
| | signaling | | | | | | |
| PLCG1 | PLC-gamma is a major substrate for | 32.6 | 0.04 | 17.0 | 0.04 | 18.4 | 0.05 |
| | heparin-binding growth factor 1 (acidic | | | | | | |
| | fibroblast growth factor)-activated | | | | | | |
| | tyrosine kinase | | | | | | |
| Q7RTU0 | Basic helix-loop-helix dimerization | 21.4 | 0.03 | 8.4 | 0.06 | 10.4 | 0.05 |
| | domain bHLH | | | | | | |
| ALDOB | Fructose-bisphosphate aldolase B; | 21.0 | 0.03 | 15.5 | 0.01 | 8.9 | 0.06 |
| | Liver-type aldolase | | | | | | |
| Q9H5P1 | Zn-finger, C-x8-C-x5-C-x3-H type | 19.9 | 0.06 | 8.6 | 0.02 | 9.1 | 0.05 |
| SYT11 | May be involved in Ca(2+)-dependent | 14.8 | 0.03 | 6.1 | 0.06 | 7.3 | 0.08 |
| | exocytosis of secretory vesicles | | | | | | |
| UBXD2 | UBX domain-containing protein 2 | 9.3 | 0.04 | 6.8 | 0.04 | 5.6 | 0.04 |
| PROZ | Appears to assist hemostasis by | 6.9 | 0.05 | 2.4 | 0.05 | 4.0 | 0.27 |
| | binding thrombin and promoting its | | | | | | |
| | association with phospholipid vesicles | | | | | | |
| PLAC8 | Placenta-specific gene 8 protein; C15 | 6.4 | 0.01 | 2.2 | 0.05 | 1.6 | 0.04 |
| | protein | | | | | | |
| Q96PN6 | ATP/GTP-binding site motif A (P- | 5.2 | 0.05 | 3.3 | 0.03 | 3.0 | 0.08 |
| | loop); Guanylate cyclase | | | | | | |
| ASTN2 | Fibronectin, type III | 5.1 | 0.03 | 3.7 | 0.06 | 4.0 | 0.16 |
| O60290 | KRAB box | 4.8 | 0.05 | 2.9 | 0.04 | 4.2 | 0.08 |
| FTCD | Folate-dependent enzyme, that has | 4.6 | 0.03 | 3.8 | 0.03 | 4.9 | 0.06 |
| | transferase and deaminase activity. | | | | | | |
| | Serves to channel one-carbon units to | | | | | | |
| | the folate pool | | | | | | |
| NFKB2 | NFκB subunits p52 and p100 | 4.3 | 0.02 | 2.8 | 0.01 | 1.9 | 0.15 |
| CTLA4 | Possibly involved in T-cell activation. | 4.1 | 0.02 | 2.7 | 0.01 | 2.4 | 0.13 |
| | Binds to B7-1 (CD80) and B7-2 | | | | | | |
| | (CD86) | | | | | | |
| PSMA1 | Proteasome subunit | 4.1 | 0.04 | 2.7 | 0.04 | 3.8 | 0.04 |
| CCL2 | Chemotactic factor that attracts | 3.4 | 0.02 | 2.9 | 0.01 | 1.3 | 0.10 |
| | monocytes and basophils but not | | | | | | |
| | neutrophils or eosinophils. | | | | | | |
| HNF4A | Transcriptionally controlled | 3.3 | 0.01 | 2.6 | 0.01 | 3.0 | 0.07 |
| | transcription factor. Binds to DNA | | | | | | |
| | sites required e.g. for the transcription | | | | | | |
| | of alpha 1-antitrypsin, and HNF1- | | | | | | |
| | alpha. | | | | | | |
| MAFF | Interacts with the upstream promoter | 3.3 | 0.01 | 2.5 | 0.00 | 2.1 | 0.04 |
| | region of the oxytocin receptor gene. | | | | | | |
| | May be involved in the cellular stress | | | | | | |
| | response | | | | | | |
| FBXO32 | Probably recognizes and binds to some | 3.3 | 0.03 | 1.9 | 0.02 | 1.2 | 0.11 |
| | phosphorylated proteins and promotes | | | | | | |
| | their ubiquitination and degradation | | | | | | |
| | during skeletal muscle atrophy | | | | | | |
| TNF | Cytokine tumour necrosis factor α | 3.3 | 0.02 | 2.4 | 0.01 | 1.2 | 0.82 |
| NPAS2 | Neuronal PAS domain protein 2; | 2.8 | 0.00 | 1.8 | 0.03 | 2.4 | 0.04 |
| ICAM3 | Ligands for the leukocyte adhesion | 2.6 | 0.04 | 1.7 | 0.03 | 2.2 | 0.05 |
| | LFA-1 protein and integrin alpha- | | | | | | |
| | D/beta-2 | | | | | | |
| Q8NC30 | transmembrane | 2.3 | 0.00 | 1.7 | 0.05 | 1.2 | 0.82 |
| Q8IUC6 | Proline-rich extensin domain | 2.3 | 0.03 | 1.4 | 0.05 | 1.3 | 0.28 |
| O94940 | SAM (and some other nucleotide) | 2.1 | 0.01 | 1.4 | 0.04 | 1.5 | 0.04 |
| | binding motif | | | | | | |
| CGI-117 | Protein CGI-117 | 1.9 | 0.00 | 1.3 | 0.01 | 1.3 | 0.17 |
| KDELR1 | Required for the retention of luminal | 1.9 | 0.04 | 1.5 | 0.02 | 1.3 | 0.20 |
| | endoplasmic reticulum proteins. | | | | | | |
| IFITM1 | Implicated in the control of cell | 1.6 | 0.02 | 1.4 | 0.01 | 1.3 | 0.24 |
| | growth. | | | | | | |
| COL7A1 | Stratified squamous epithelial | 1.5 | 0.05 | 1.4 | 0.02 | 1.9 | 0.31 |
| | basement membrane protein that form | | | | | | |
| | anchoring fibrils which may contribute | | | | | | |
| | to epithelial basement membrane | | | | | | |
| | organization and adherence by | | | | | | |
| | interacting with extracellular matrix | | | | | | |
| | (ECM) proteins such as type IV | | | | | | |
| | collagen | | | | | | |

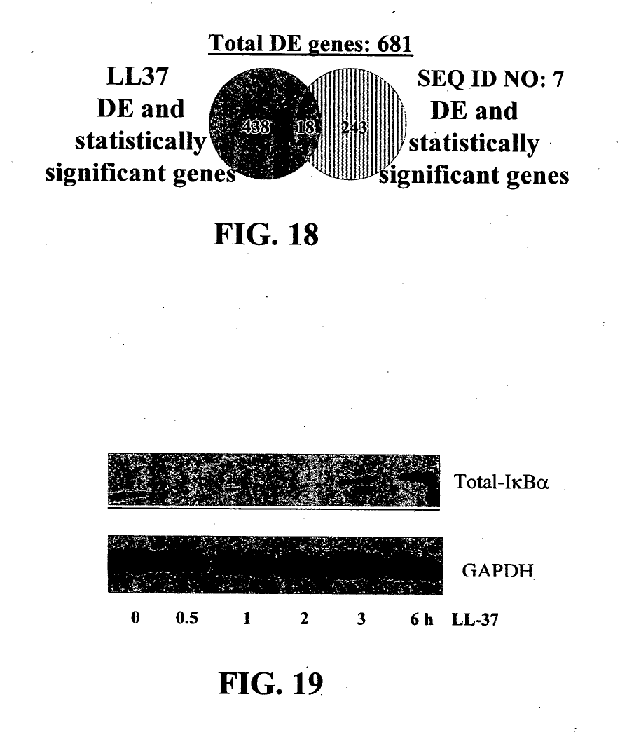

The data in FIGS. 13 and 18 also indicate that SEQ ID NO: 7 has both overlapping and distinct activities compared to LL-37.

The overall data extrapolated from this study indicates that the efficacy of host defense peptides can be improved significantly and specifically in order to obtain novel therapeutics that not only have anti-inflammatory properties, but also can specifically modulate responses in critical pathways that are involved in host defenses in pathogenesis.

### EXAMPLE 15

### Mechanisms of Action of Human Host Defense Peptide LL-37

LL-37 is a human cationic host defense peptide that is an essential component of innate immunity. It is a multifimctional modulator of innate immune responses demonstrating an ability to modulate gene regulation in certain cells, to alter cytokine expression in macrophages, to demonstrate chemotactic activity for neutrophils, monocytes and T cells, and to neutralize the endotoxic effects of lipopolysaccharide (LPS). However, the underlying mechanisms determining these effects of LL-37 were not clear. The general aim of this example is to determine how LL-37 affects initial cell signaling and to link cell signaling to some of these observed biological functions that are described herein such as the anti-endotoxin property and the ability to modulate cytokine and chemokine production in a human monocytic THP-1 cell line and human peripheral blood mononuclear cells (PBMCs).

To meet these goals, a variety of specific approaches were applied: (1) Assessing, by RT-PCR and ELISA, the ability of LL-37 to block the LPS-induced upregulation of transcription of certain genes and production of pro-inflammatory cytokines, respectively, in THP-1 cells and PBMCs; (2) Degradation of IκBα (known to be an NF-κB negative regulator) and translocation of the transcription factor NFκB were examined in the LPS-stimulated human THP-1 cell line in the presence of LL-37; (3) Since LPS and cytokine interleukin-1α (IL-1α) share similar signaling transduction pathways, it was interesting to compare the effects of LL-37 in modulating cytokine (IL-6) and chemokine (MCP-3) production in LPS- or IL-1α-stimualted human PBMCs; (4) Inhibitors (including oxidized ATP, an agonist of the P2X7 receptor, pertussis toxin, which inhibits G-protein coupled receptors, and LY294002, a PI3 Kinase inhibitor) were used to test if the synergistic effects of LL-37 were mediated by these receptors or pathways; (5) Western blots were performed to evaluate protein kinase B (Akt) phosphorylation and the activation of downstream transcription factors e.g., assessing the phosphorylation of cAMP-responsive element binding protein (CREB) and translocation of NFκB subunit p50 in human PBMCs. These analyses are designed to test if the PI3K-Akt-IκBα-NFκB and PI3K-Akt-CREB pathways are involved in LL-37-induced modulation of cytokine and chemokine release.

Methods

Western blotting—THP-1 cells (1×10⁶ cells per condition) or PBMCs (5×10⁶ cells per condition) were stimulated by adding LPS, LL-37 or endotoxin-free water as a vehicle control, and incubated for 20 min at 37° C., 5% CO₂. After stimulation, the cells were centrifuged, washed once with ice-cold PBS with 1 mM vanadate and nuclear extracts were isolated using NE-PER Nuclear and Cytoplasmic Extraction Reagents Kit (Pierce) according to the manufacturer's instructions. The lysates were assayed for protein concentration using a BCA assay (Pierce). 7.5 μg nuclear lysate and 15 μg cytoplasm lysate were loaded per lane, resolved on a 8-10% SDS-PAGE at 120 V for 1 hr, and transferred to Immuno-blot PVDF membranes (Bio-Rad) for 75 min at 100 V. Immunoblot was performed using 1/1000 dilution of anti-p50, anti-total IκBα, anti-p-IκBα and anti-Akt (cell signaling) monoclonal antibodies in TBST/milk for 1 hour at room temperature. Membranes were washed for 30 min in TBST and then incubated with a 1/5000 dilution of HRP-conjugated goat anti-mouse or anti-rabbit Ab (in TBST/milk) for 1 hour. The membranes were washed for 30 min in TBST and developed with chemiluminescence peroxidase substrate (Sigma-Aldrich), according to manufacturer's instructions. The blots were reprobed with anti-total Akt or anti-GAPDH antibody as loading control.

Detection of cytokines—Fresh human PBMCs were plated at 8×10⁵ cells in 1 ml of RPMI 1640 media (supplemented with 10% (v/v) heat-inactivated FBS, 1% (v/v) L-glutamine, 1 nM sodium pyruvate) respectively in 24 well plates. Cells were then incubated in media for periods of 24 hours in the presence of Pseudomonas aeruginosa PAO1 strain H103 LPS, LL-37, IL-1α (at the concentrations stated above), or endotoxin-free water as a vehicle control, in at least triplicate. Supernatants were collected and stored at −20° C. until use. The concentrations of IL-6 (eBioscience) and MCP-3 (R&D system) in the supernatants were measured using commercially prepared ELISA plates in accordance to the manufacturer's suggestion.

Results

LL-37 alone (20 μg/ml) caused IκBα degradation: protein levels of total IκBα diminished within 30 min and returned to control levels by 60 min in THP-1 cells, indicating that LL-37 may directly modulate elements of the LPS signaling pathway. Results are representative of three independent experiments (FIG. 19).

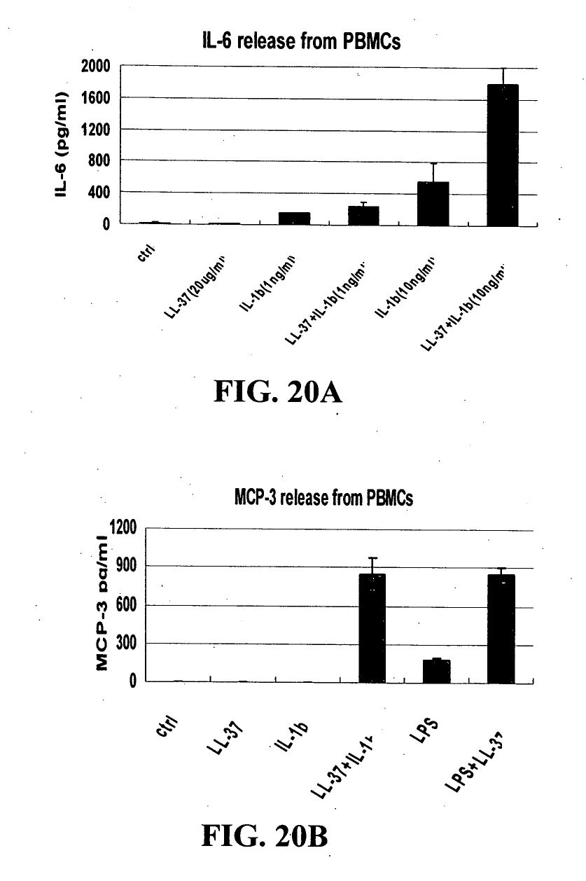

Fresh isolated human PBMCs were incubated with IL-1β (10 ng/ml) or LPS (100 ng/ml) in absence or presence of LL-37 (20 ug/ml) for 24 hours. IL-6 and MCP-3 ELISA were performed to measure the level of protein release (FIGS. 20A and 20B). For IL-6 production, stimulation with IL-1β resulted in an increase in IL-6 production, which could be significantly enhanced by the simultaneous addition of LL-37 even though LL-37 alone did not induce IL-6 release in THP-1 and PBMCs. In contrast, LL-37 inhibited LPS-triggered IL-6 release. Similar to IL-6, synergistic effects between IL-1β and LL-37 were also demonstrated for MCP-3 production. More importantly, LL-37 dramatically increased release of the MCP-3 in the presence of LPS, while LPS alone induced only low, but appreciable levels of MCP-3. Results are representative of 6 donors.

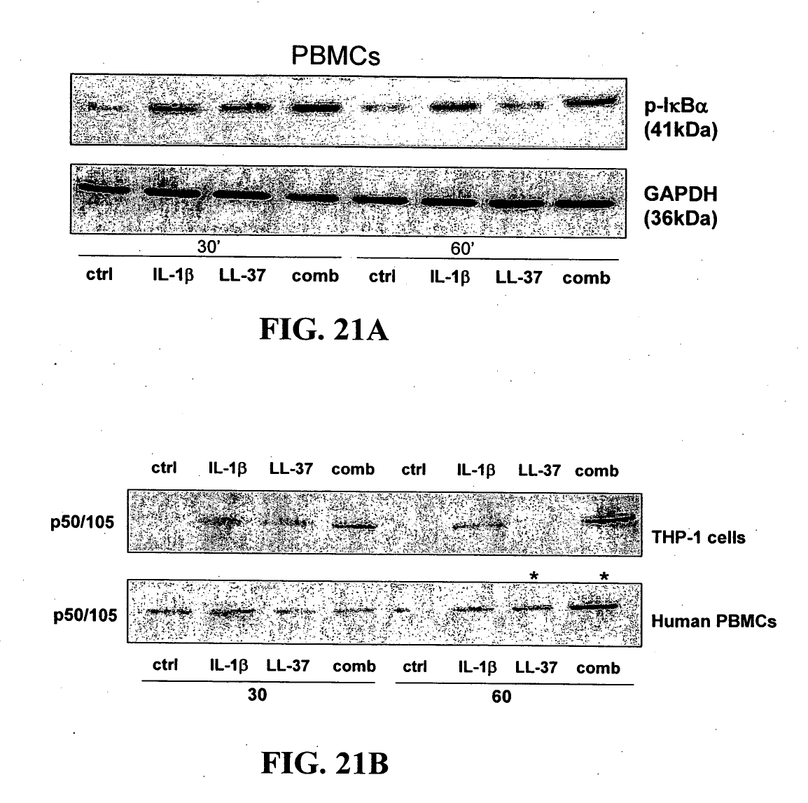

Cells were incubated with LL-37 alone or IL-1β (10 ng/ml) with or without LL-37 (20 μg/ml). Western blots were performed for cytoplasm protein and nuclear protein, showing that combined treatment of LL-37 and IL-1β showed higher IκBα phosphorylation after 30 min and p50 nuclear translocation after 60 min than LL-37 or IL-1β treatment alone in human PBMCs (FIG. 21A). Similar translocation results were also observed in THP-1 cells at an earlier time point (20 min after treatment). In addition, LL-37 alone induced NFκB subunit p50 translocation in both human PBMCs and THP-1 cells (FIG. 21B). Results are representative of three independent experiments for THP-1 cells and three donors for human PBMCs.

The immunomodulatory effects of LL-37 have been proposed to be dependent on signaling through a number of receptors, including the G-protein coupled receptor FPRL-1 and the purinergic receptor P2X7. Human PBMCs were pre-incubated with pertussis toxin (PTx, a G-protein coupled receptor inhibitor), oxidised ATP (OATP, a P2X7 receptor inhibitor) before exposure to LL-37 with or without IL-1β. MCP-3 production that resulted from combined treatment with LL-37 and IL-1β was dramatically inhibited by pre-treatment of PTx and partially blocked by oATP preincubation.

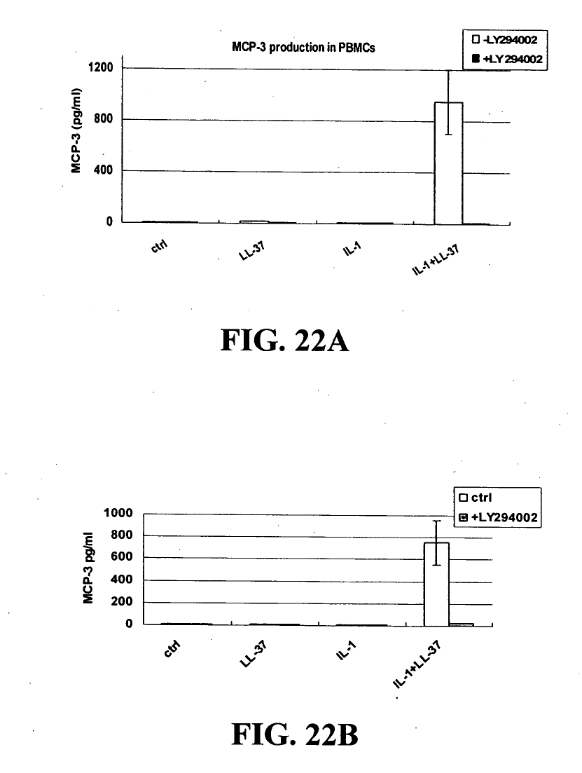

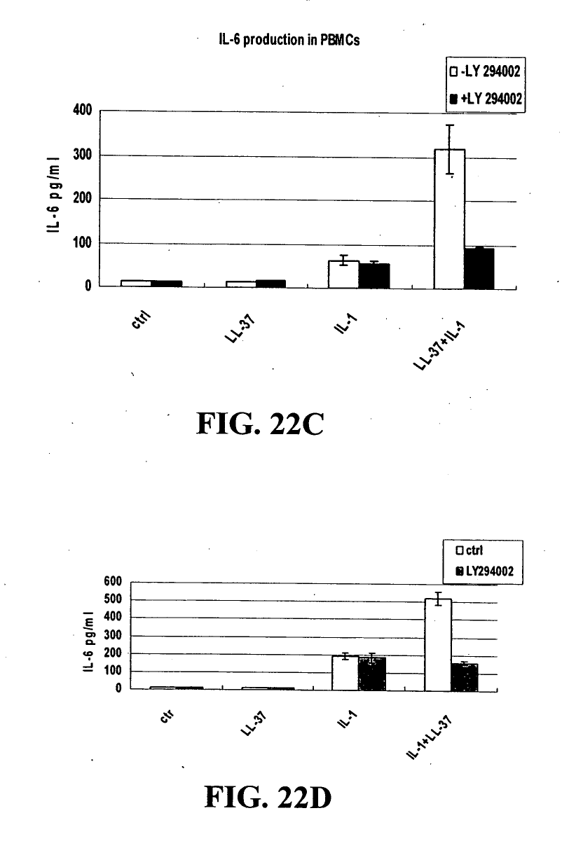

Since activated G-protein coupled receptor can lead to the elevated signaling of phosphatidylinositol 3-kinase (PI3 kinase) pathway, the role of PI3 kinase signaling in the above effects was further examined. Human PBMCs were pre-treated with PI3 kinase inhibitor, LY294002 (25 μM) for lh and following incubation with IL-1β (10 ng/ml) in presence or absence of LL-37 (20 μg/ml) for 24 hours (FIGS. 22A-22D). The LL-37 plus IL-1β mediated production of IL-6 and MCP-3 was significantly inhibited by LY294002 pre-incubation, indicating that PI3 kinase plays a role in LL-37-induced modulation of cytokine and chemokine production. Results are two representatives of three donors for human PBMCs.

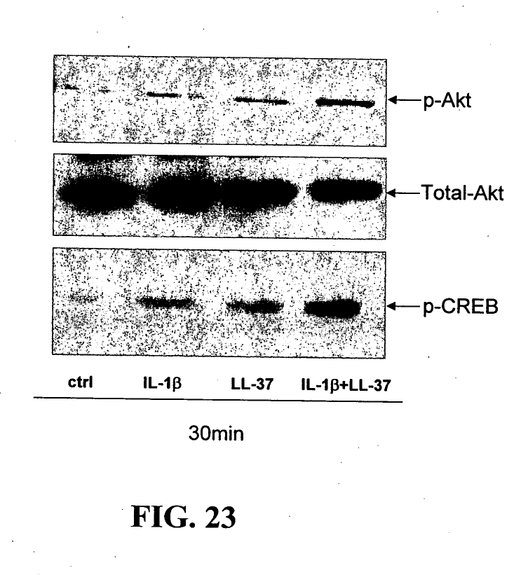

Activation of PI3 kinase causes activation of a number of intracellular signal transduction pathways, including phosphorylation of the downstream target protein kinase B (Akt). Cytoplasm protein was isolated from human PBMCs treated with LL-37 with or without IL-1β. Western blot analysis showed that phosphorylation of Akt was observed in human PBMCs after exposure to LL-37 for 30 min (FIG. 23, top panel). Furthermore, activation of Akt was augmented by the presence of IL-1β (10 ng/ml). Results are representatives of three donors for human PBMCs.

The transcription factor CREB is downstream of the PI3 kinase→Akt pathway that leads to phosphorylation and activation of CREB and resulting in transcriptional up-regulation of a broad array of cellular responses. Western blot analysis showed that phosphorylation of CREB was observed in human PBMCs after exposure to LL-37 for 30 min and 60 min (FIG. 23, bottom panel). Furthermore, activation of Akt and CREB was augmented by the presence of IL-1β (10 ng/ml).

Although the invention has been described with reference to the presently preferred embodiment, it should be understood that various modifications can be made without departing from the spirit of the invention. Accordingly, the invention is limited only by the following claims.

What is claimed is:

1. A method of identifying an agent that is capable of selectively enhancing innate immunity comprising contacting a cell containing one or more genes that encode a polypeptide involved in innate immunity and protection against an infection, with an agent of interest, wherein expression of the one or more genes in the presence of the agent is modulated as compared with expression of the one or more genes in the absence of the agent, and wherein the modulated expression results in enhancement of innate immunity.

2. An agent identified by the method of claim 1.

3. The method of claim 1, wherein the agent does not stimulate a septic reaction.

4. The method of claim 3, wherein the agent stimulates expression of the one or more genes, thereby selectively enhancing innate immunity.

5. The method of claim 4, wherein the one or more genes are any gene shown in Table 69.

6. The method of claim 4, wherein the one or more genes encode G-coupled protein receptors that initiate signaling from extracellular ligands.

7. The method of claim 6, wherein the one or more genes are selected from the group consisting of GPR55, GPR6, GPR30, GPCR42, CASR, and EDG2.

8. The method of claim 4, wherein the one or more genes encode chemokines or interleukins that attract immune cells.

9. The method of claim 8, wherein one or more genes are delected from the group consisting of MCP-1, MCP-3, IL-8, CXCL-1, IL-17C, and IL-19.

10. The method of claim 4, wherein the one or more genes encode receptors for chemokines.

11. The method of claim 10, wherein the gene is CCR7.

12. The method of claim 4, wherein the one or more genes encode transcription factors that mediate selective gene expression.

13. The method of claim 12, wherein the one or more genes are selected from the group consisting of JAK1, STAT1, ELF1, Q9Y4C1, ETV4, POU1F1, ZNF254, ZNF292, ZNF78L1, HOXD3, and DLX5.

14. The method of claim 4, wherein the one or more genes encode tyrosine-protein kinase or tyrosine-protein kinase receptors.

15. The method of claim 14, wherein the one or more genes are selected from the group consisting of MAP2K6, NTRK3, PLCG1, EFNA2, and NCK1.

16. The method of claim 4, wherein the one or more genes encode adhesion molecules that mediate cell attachment and interaction.

17. The method of claim 16, wherein the one or more genes encode adhesion molecules of the ICAM, NCAM families, and PTPRF.

18. The method of claim 17, wherein the one or more genes are selected from the group consisting of ICAM3, NCAM2, and PTPRF.

19. The method of claim 4, wherein the one or more genes are involved in actin polymerization or cytoskeletal remodeling.

20. The method of claim 19, wherein the one or more genes are selected from the group consisting of Integrin-α, EPHA4, ARHGAP6, and DST.

21. The method of claim 4, wherein the one or more genes encode regulators of transcription factors.

22. The method of claim 21, wherein the one or more genes are selected from the group consisting of TRIP4, GMEB2, GSK3B, ARNT, BACH1, ARID3A, HIPK2, POLR2D, TGIF, SSBP3, and FYB.

23. The method of claim 4, wherein the one or more genes encode transmembrane receptors and adapters of signaling pathways.

24. The method of claim 23, wherein the one or more genes are selected from the group consisting of WNT5B, FZD 10, TIRAP, and REPS 1.

25. The method of claim 4, wherein the one or more genes encode proteins involved in antiviral activity.

26. The method of claim 25, wherein the one or more genes are selected from the group consisting of IFNA2, STAT1, MNDA, and IFNA2.

27. The method of claim 4, wherein the agent stimulates the JAK-STAT pathway.

28. The method of claim 27, wherein the agent stimulates expression of one or more genes selected from the group consisting of JAK2, STAT1, STAT3, SOCS 1, and IL-19.

29. The method of claim 4, wherein the agent stimulates the P13K pathway.

30. The method of claim 29, wherein the agent stimulates expression of one or more genes selected from the group consisting of BACH2/PIK3CB, Akt, CREB, IL-6, and MCP-3.

31. The method of claim 4, wherein the agent stimulates the ERK1/2 mitogen activated kinase pathway.

32. The method of claim 31, wherein the agent stimulates expression of one or more genes selected from the group consisting of MAP3K1 and PP2A

33. The method of claim 4, wherein the agent stimulates the p38 mitogen activated kinase pathway.

34. The method of claim 33, wherein the agent stimulates expression of one or more genes selected from the group consisting of MINK1/MAP4K6, MAP2K6, and MAP2K4.

35. The method of claim 4, wherein the agent transiently stimulates the NFYB pathway.

36. The method of claim 35, wherein the agent stimulates expression of one or more genes selected from the group consisting of TIRAP, NFκB2 (p52), DUSP14, ICAM3, TRIP4, MMP17, ITGB4, ZNF36, ZNF251, BNIP1, CD226, NRXN1, and TNC.

37. The method of claim 4, wherein the agent stimulates the AP-1, JNK or Wnt pathways.

38. The method of claim 37, wherein the agent stimulates expression of one or more genes selected from the group consisting of TRIP4, TIRAP, HIPK2, GSK3B, and FZD10.

39. A method of identifying a pattern of gene expression for identification of an agent that selectively enhances innate immunity comprising:
contacting a cell containing one. or more genes that encode a polypeptide involved in innate immunity and defense against infections, with an agent of interest, wherein expression of the one or more genes in the presence of the agent is modulated as compared with expression of the one or more genes in the absence of the agent, and wherein the modulated expression results in. enhancement of innate immunity.

40. The method of claim 39, wherein the modulated expression is a marker of enhancement of innate immunity.

41. The method of claim 40, further comprising determining the efficacy of compounds that enhance innate immunity.

42. The method of claim 40, wherein the one or more genes are any gene shown in Table 69.

43. The method of claim 42, wherein the one or more genes express IL-8, IL-6, IL-19, CXCL-1, MCP-3, or MCP-1.

44. The method of claim 39, wherein the modulated expression occurs in the presence of a bacterial signature molecule.

45. The method of claim 44, wherein the bacterial signature molecule is a Toll-like receptor agonist.

46. The method of claim 45, wherein the Toll-like receptor agonist is selected from the group consisting of bacterial lipopolysaccharide, lipoteichoic acid, and CpG bacterial signature DNA.

47. The method of claim 44, wherein the one or more genes are any gene shown in Table 71.

48. A method of identifying an agent that is capable of selectively enhancing innate immunity in the presence of an infection or bacterial signature molecule comprising:
contacting a cell containing one or more genes that encode a polypeptide involved in innate immunity, with an agent of interest in the presence of a bacterial signature molecule, wherein expression of the one or more genes in the presence of the agent and bacterial signature molecule is modulated as compared with expression of the one or more genes in the absence of the agent and bacterial signature molecule, and wherein the modulated expression results in enhancement of innate immunity.

49. An agent identified by the method of claim 48.

50. The method of claim 48, wherein the bacterial signature molecule is a Toll-like receptor agonist.

51. The method of claim 50, wherein the Toll-like receptor agonist is selected from the group consisting of bacterial lipopolysaccharide, lipoteichoic acid, and CpG bacterial signature DNA.

52. The method of claim 51, wherein the one or genes are any gene shown in Table 71.

53. The method of claim 52, wherein the agent does not stimulate a septic reaction.

54. The method of claim 48, wherein the agent has anti-endotoxic activity.

55. The method of claim 54, wherein the one ore more genes are selected from the group consisting of GPD1, Q8NI35, FEZ2, NRXN1, PLCG1, Q7RTU0, ALDOB, Q9H5P1, SYT11, UBXD2, PROZ, PLAC8, Q96PN6, ASTN2, O60290, FTCD, NFKB2, CTLA4, PSMA 1, CCL2, HNF4A, MAFF, FBXO32, TNFα, NPAS2, ICAM3, Q8NC30, Q8IUC6, O94940, CGI-117, KDELR1, IFITM1 and COL7A1.

56. The method of claim 48, wherein the agent stimulates transient IKcBa degradation or transient NFκB subunit p50 translocation.

57. The method of claim 48, further comprising contacting the cell with IL-1β.

58. The method of claim 48, wherein the one-or more genes encode chemokines.

59. The method of claim 58, wherein the one or more genes are selected from the group consisting of CCL20, CCL23, IL-6, and MCP-3.

60. The method of claim 48, wherein the one or more genes encode cytokine receptors.

61. The method of claim 60, wherein the one or more genes are EBI3 or IL7R.

62. The method of claim 48, wherein the one or more genes encode factors involved in lymphocyte activation.

63. The method of claim 62, wherein the one or more genes are selected from the group consisting of SLAMF1, CD58, and IL32.

64. The method of claim 48, wherein the one or more genes encode regulators of signal transduction.

65. The method of claim 64, wherein the one or more genes are selected from the group consisting of MAP2K2, DUSP5, MAPK8IP3, RIN2, RANBP9, IP3 3-kinase A, BATF, IRAK3, NM1, SP3, RAP2C, PNRC1, NEK1, CHC1, ZNF219, ZNF593, WIF1, PIM2, CD79A, and LATS2.

66. The method of claim 48, wherein the one or more genes encode substrate transporters.

67. The method of claim 66, wherein the one or more genes are SLC23A3 or SLC17A5.

68. The method of claim 48, wherein the one or more genes encode apoptosis regulators.

69. The method of claim 68, wherein the one or more genes are selected from the group consisting of BOK, BIRC3, TNFRSF6, and CASP9.

70. The method of claim 48, wherein the one or more genes encode genes associated with plasma membrane.

71. The method of claim 70, wherein the one. or more genes are selected from the group consisting of STIM1, BPAG1, PTPN4, TRIM36, SDK1, and FNDC5.

72. The method of claim 48, wherein the one or more genes encode genes involved in selective ion transport and in mediating selective ion-channels.

73. The method of claim 72, wherein the one or more genes are selected from the group consisting of VGCNL1, TRPC5, CACNA1B, KCNA6, KCNJ2, KCNA10, and AQP9.

74. The method of claim 48, wherein the one or more genes encode growth modulating genes or genes involved in wound healing.

75. The method of claim 74, wherein the one or more genes are FGF10 or AREG.

76. The method of claim 48, wherein the one or more genes encode inflammatory mediators.

77. The method of claim 76, wherein the one or more genes are selected from the group consisting of PTGS2, SOD2, TNFAIP8, and TNIP3.

78. The method of claim 48, further comprising contacting the cell with IL-1β, wherein the agent stimulates the PI3 kinase pathway.

79. The method of claim 78, wherein the agent stimulates transient IκBα phosphorylation and p50 nuclear translocation.

80. The method of claim 78, wherein the one or more genes encodes a G-protein coupled receptor or a purinergic receptor.

81. The method of claim 78, wherein the purinergic receptor is P2X7.

82. The method of claim 78, wherein the agent further stimulates phosphorylation of Akt.

83. The method of claim 82, wherein the phosphorylation of Akt stimulates activation of CREB.

84. A method of identifying an agent that selectively reduces inflammation comprising: contacting a cell containing one or more genes that encode a polypeptide involved in sepsis, with an agent of interest, wherein the agent reduces expression of the one or more genes compared with expression of the one or more genes in the absence of the agent.

85. The method of claim 84, wherein the one or more genes are selected from the group consisting of GPD1, Q8NI35, FEZ2, NRXN, PLCG1, Q7RTU0, ALDOB, Q9H5P1, SYTI 11, UBXD2, PROZ, PLAC8, Q96PN6, ASTN2, O60290, FTCD, NFKB2, CTLA4, PSMA1, CCL2, HNF4A, MAFF, FBXO32, TNF, NPAS2, ICAM3, Q8NC30, Q81UC6, O94940, CGI-117, KDELR1, IFITM1, and COL7A1.


SEQUENCE LISTING (96 sequences)

SEQ ID NO: 1 - protein, 37 aa, Homo sapiens
LLGDFFRKSKEKIGKEFKRIVQRIKDFLRNLVPRTES

SEQ ID NO: 2 - protein, 13 aa, Bovine
ILPWKWPWWPWRR

SEQ ID NO: 3 - protein, 12 aa, Bovine
RLARIVVIRVAR

SEQ ID NO: 4 - protein, 14 aa, Artificial sequence
  Cationic peptide
  MISC_FEATURE (1)..(2): Xaa is independently R, L or K and one or both may be present
  MISC_FEATURE (3)..(3): Xaa is one of C, S or A
  MISC_FEATURE (4)..(4): Xaa is one of R or P
  MISC_FEATURE (6)..(6): Xaa is one of A or V
  MISC_FEATURE (8)..(8): Xaa is one of A or V
  MISC_FEATURE (11)..(11): Xaa is one of V or W
  MISC_FEATURE (12)..(12): Xaa is one of C, S or A
  MISC_FEATURE (13)..(14): Xaa is independently R, L or K and one or both may be present
XXXXIXPXIPXXXX

SEQ ID NO: 5 - protein, 13 aa, Artificial sequence
  Cationic peptide
LLCRIVPVIPWCK

SEQ ID NO: 6 - protein, 14 aa, Artificial sequence
  Cationic peptide
LRCPIAPVIPVCKK

SEQ ID NO: 7 - protein, 13 aa, Artificial sequence
  Cationic peptide
KSRIVPAIPVSLL

SEQ ID NO: 8 - protein, 13 aa, Artificial sequence
  Cationic peptide
KKSPIAPAIPWSR

SEQ ID NO: 9 - protein, 14 aa, Artificial sequence
  Cationic peptide
RRARIVPAIPVARR

SEQ ID NO: 10 - protein, 13 aa, Artificial sequence
  Cationic peptide
LSRIAPAIPWAKL

SEQ ID NO: 11 - protein, 16 aa, Artificial sequence
  Cationic peptide
  MISC_FEATURE (1)..(2): Xaa is independently D, E, S, T or N and one or both may be present
  MISC_FEATURE (4)..(5): Xaa is P, G or D and one or both may be present
  MISC_FEATURE (6)..(6): Xaa is one of G, A, V, L, I or Y
  MISC_FEATURE (8)..(8): Xaa is one of R, K or H
  MISC_FEATURE (9)..(10): Xaa is P, G or D and one or both may be present
  MISC_FEATURE (11)..(11): Xaa is one of S, T, C, M or R
  MISC_FEATURE (12)..(12): Xaa is one of G, A, V, L, I or Y
  MISC_FEATURE (14)..(14): Xaa is one of G, A, V, L, I or Y
  MISC_FEATURE (15)..(16): Xaa is D, E, S, T or N and one or both may be present
XXLXXXKXXXXXPXXX

SEQ ID NO: 12 - protein, 13 aa, Artificial sequence
  Cationic peptide
DLPAKRGSAPGST

SEQ ID NO: 13 - protein, 14 aa, Artificial sequence
  Cationic peptide
SELPGLKHPCVPGS

SEQ ID NO: 14 - protein, 14 aa, Artificial sequence
  Cationic peptide
TTLGPVKRDSIPGE

SEQ ID NO: 15 - protein, 13 aa, Artificial sequence
  Cationic peptide
SLPIKHDRLPATS

SEQ ID NO: 16 - protein, 12 aa, Artificial sequence
  Cationic peptide
ELPLKRGRVPVE

SEQ ID NO: 17 - protein, 14 aa, Artificial sequence
  Cationic peptide
NLPDLKKPRVPATS

SEQ ID NO: 18 - protein, 19 aa, Artificial sequence
  Cationic peptide
  MISC_FEATURE (1)..(4): Xaa is chosen from A, P or R and one, two, three or all four may be present
  MISC_FEATURE (5)..(6): Xaa is an aromatic amino acid (F, Y and W)
  MISC_FEATURE (7)..(7): Xaa is one of P or K
  MISC_FEATURE (8)..(9): Xaa is chosen from A, P, Y or W and one, both or none may be present
  MISC_FEATURE (11)..(12): Xaa is chosen from A, P, Y or W and one, both or none may be present
  MISC_FEATURE (14)..(15): Xaa is chosen from A, P, Y or W and one, both or none may be present
  MISC_FEATURE (16)..(18): Xaa is chosen from R or P and one, two or three may be present
XXXXXXXXXWXXWXXXXXK

SEQ ID NO: 19 - protein, 16 aa, Artificial sequence
  Cationic peptide
RPRYPWWPWWPYRPRK

SEQ ID NO: 20 - protein, 13 aa, Artificial sequence
  Cationic peptide
RRAWWKAWWARRK

SEQ ID NO: 21 - protein, 14 aa, Artificial sequence
  Cationic peptide
RAPYWPWAWARPRK

SEQ ID NO: 22 - protein, 16 aa, Artificial sequence
  Cationic peptide
RPAWKYWWPWPWPRRK

SEQ ID NO: 23 - protein, 14 aa, Artificial sequence
  Cationic peptide
RAAFKWAWAWWRRK

SEQ ID NO: 24 - protein, 12 aa, Artificial sequence
  Cationic peptide
RRRWKWAWPRRK

SEQ ID NO: 25 - protein, 20 aa, Artificial sequence
  Cationic peptide
  MISC_FEATURE (1)..(2): Xaa is R or K and one or both may be present
  MISC_FEATURE (3)..(3): Xaa is a polar or charged amino acid (S, T, M, N, Q, D, E, K, R and H)
  MISC_FEATURE (4)..(4): Xaa is C, S, M, D or A
  MISC_FEATURE (5)..(5): Xaa is F, I, V, M or R
  MISC_FEATURE (6)..(7): Xaa is R or K and one or both may be present
  MISC_FEATURE (9)..(9): Xaa is C, S, M, D or A
  MISC_FEATURE (10)..(10): Xaa is F, I, V, M or R
  MISC_FEATURE (13)..(13): Xaa is F, I, V, M or R
  MISC_FEATURE (14)..(14): Xaa is C, S, M, D or A
  MISC_FEATURE (15)..(15): Xaa is F, I, V, M or R
  MISC_FEATURE (16)..(17): Xaa is R or K and one or both may be present
  MISC_FEATURE (18)..(18): Xaa is C, S, M, D or A
  MISC_FEATURE (19)..(20): Xaa is R or K and one or both may be present
XXXXXXXVXXRGXXXXXXXX

SEQ ID NO: 26 - protein, 19 aa, Artificial sequence
  Cationic peptide
RRMCIKVCVRGVCRRKCRK

SEQ ID NO: 27 - protein, 18 aa, Artificial sequence
  Cationic peptide
KRSCFKVSMRGVSRRRCK

SEQ ID NO: 28 - protein, 19 aa, Artificial sequence
  Cationic peptide
KKDAIKKVDIRGMDMRRAR

SEQ ID NO: 29 - protein, 18 aa, Artificial sequence
  Cationic peptide
RKMVKVDVRGIMIRKDRR

SEQ ID NO: 30 - protein, 17 aa, Artificial sequence
  Cationic peptide
KQCVKVAMRGMALRRCK

SEQ ID NO: 31 - protein, 20 aa, Artificial sequence
  Cationic peptide
RREAIRRVAMRGRDMKRMRR

SEQ ID NO: 32 - protein, 17 aa, Artificial sequence
  Cationic peptide
  MISC_FEATURE (1)..(1): Xaa is R or K
  MISC_FEATURE (2)..(2): Xaa is a polar or charged amino acid (S, T, M, N, Q, D, E, K, R and H)
  MISC_FEATURE (3)..(3): Xaa is one of C, S, M, D or A
  MISC_FEATURE (4)..(4): Xaa is one of F, I, V, M or R
  MISC_FEATURE (5)..(6): Xaa is R or K and one or both may be present
  MISC_FEATURE (8)..(8): Xaa is one of A, I, S, M, D or R
  MISC_FEATURE (9)..(9): Xaa is one of F, I, V, M or R
  MISC_FEATURE (12)..(12): Xaa is one of F, I, V, M or R
  MISC_FEATURE (13)..(13): Xaa is one of A, I, S, M, D or R
  MISC_FEATURE (14)..(14): Xaa is one of F, I, V, M or R
  MISC_FEATURE (15)..(15): Xaa is R or K
  MISC_FEATURE (16)..(16): Xaa is one of C, S, M, D or A
  MISC_FEATURE (17)..(17): Xaa is R or K
XXXXXXVXXRGXXXXXX

SEQ ID NO: 33 - protein, 18 aa, Artificial sequence
  Cationic peptide
RTCVKRVAMRGIIRKRCR

SEQ ID NO: 34 - protein, 19 aa, Artificial sequence
  Cationic peptide
KKQMMKRVDVRGISVKRKR

SEQ ID NO: 35 - protein, 17 aa, Artificial sequence
  Cationic peptide
KESIKVIIRGMMVRMKK

SEQ ID NO: 36 - protein, 17 aa, Artificial sequence
  Cationic peptide
RRDCRRVMVRGIDIKAK

SEQ ID NO: 37 - protein, 19 aa, Artificial sequence
  Cationic peptide
KRTAIKKVSRRGMSVKARR

SEQ ID NO: 38 - protein, 18 aa, Artificial sequence
  Cationic peptide
RHCIRRVSMRGIIMRRCK

SEQ ID NO: 39 - protein, 31 aa, Artificial sequence
  Cationic peptide
  MISC_FEATURE (2)..(2): Xaa is a polar amino acid (C, S, T, M, N and Q)
  MISC_FEATURE (4)..(4): Xaa is one of A, L, S or K
  MISC_FEATURE (6)..(6): Xaa is one of A, L, S or K
  MISC_FEATURE (11)..(11): Xaa is one of A, L, S or K
  MISC_FEATURE (16)..(16): Xaa is one of A, L, S or K
  MISC_FEATURE (16)..(31): Xaa is amino acids chosen from G, A, V, L, I, P, F, S, T, K and H and one to seventeen may be present
KXKXFXKMLMXALKKXXXXXXXXXXXXXXXX

SEQ ID NO: 40 - protein, 28 aa, Artificial sequence
  Cationic peptide
KCKLFKKMLMLALKKVLTTGLPALKLTK

SEQ ID NO: 41 - protein, 26 aa, Artificial sequence
  Cationic peptide
KSKSFLKMLMKALKKVLTTGLPALIS

SEQ ID NO: 42 - protein, 27 aa, Artificial sequence
  Cationic peptide
KTKKFAKMLMMALKKVVSTAKPLAILS

SEQ ID NO: 43 - protein, 32 aa, Artificial sequence
  Cationic peptide
KMKSFAKMLMLALKKVLKVLTTALTLKAGLPS

SEQ ID NO: 44 - protein, 25 aa, Artificial sequence
  Cationic peptide
KNKAFAKMLMKALKKVTTAAKPLTG

SEQ ID NO: 45 - protein, 26 aa, Artificial sequence
  Cationic peptide
KQKLFAKMLMSALKKKTLVTTPLAGK

SEQ ID NO: 46 - protein, 26 aa, Artificial sequence
  Cationic peptide
  MISC_FEATURE (1)..(26): Xaa at residues 4, 7, 8, 10, 11,14, 15 is a hydrophobic amino acid
  MISC_FEATURE (1)..(26): Xaa at residues 5, 6, 9, 12, 13 is a hydrophilic amino acid
KWKXXXXXXXXXXXXIFHTALKPISS

SEQ ID NO: 47 - protein, 26 aa, Artificial sequence
  Cationic peptide
KWKSFLRTFKSPVRTIFHTALKPISS

SEQ ID NO: 48 - protein, 26 aa, Artificial sequence
  Cationic peptide
KWKSYAHTIMSPVRLIFHTALKPISS

SEQ ID NO: 49 - protein, 26 aa, Artificial sequence
  Cationic peptide
KWKRGAHRFMKFLSTIFHTALKPISS

SEQ ID NO: 50 - protein, 26 aa, Artificial sequence
  Cationic peptide
KWKKWAHSPRKVLTRIFHTALKPISS

SEQ ID NO: 51 - protein, 26 aa, Artificial sequence
  Cationic peptide
KWKSLVMMFKKPARRIFHTALKPISS

SEQ ID NO: 52 - protein, 26 aa, Artificial sequence
  Cationic peptide
KWKHALMKAHMLWHMIFHTALKPISS

SEQ ID NO: 53 - protein, 26 aa, Artificial sequence
  Cationic peptide
KWKSFLRTFKSPVRTVFHTALKPISS

SEQ ID NO: 54 - protein, 26 aa, Artificial sequence
  Cationic peptide
KWKSYAHTIMSPVRLVFHTALKPISS

SEQ ID NO: 55 - DNA, 20 bp, Artificial sequence
  Primer
gtccctgtatgcctctggtc

SEQ ID NO: 56 - DNA, 19 bp, Artificial sequence
  Primer
gatgtcacgcacgatttcc

SEQ ID NO: 57 - DNA, 19 bp, Artificial sequence
  CpG oligonucleotide
tcatgacgttcctgacgtt

SEQ ID NO: 58 - DNA, 20 bp, Artificial sequence
  nonCpG oligonucleotide
ttcaggactttcctcaggtt

SEQ ID NO: 59 - DNA, 21 bp, Homo sapiens
tcatagcagccaccttcattc

SEQ ID NO: 60 - DNA, 20 bp, Homo sapiens
tagcgcagattcttgggttg

SEQ ID NO: 61 - DNA, 22 bp, Homo sapiens
tgtcctttctcagagtggttct

SEQ ID NO: 62 - DNA, 21 bp, Homo sapiens
tgcttccatagggacatcata

SEQ ID NO: 63 - DNA, 21 bp, Homo sapiens
acctgaaccttccaaagatgg

SEQ ID NO: 64 - DNA, 20 bp, Homo sapiens
gcgcagaatgagatgagttg

SEQ ID NO: 65 - DNA, 21 bp, Homo sapiens
gtgcagagggttgtggagaag

SEQ ID NO: 66 - DNA, 21 bp, Homo sapiens
ttctcccgtgcaatatctagg

SEQ ID NO: 67 - DNA, 21 bp, Artificial sequence
  Primer
cttttcttacaccgcgaggaa

SEQ ID NO: 68 - DNA, 20 bp, Artificial sequence
  Primer
gcagaggctgctggtctcat

SEQ ID NO: 69 - DNA, 24 bp, Artificial sequence
  Primer
tgactgctgtcttggatacacaga

SEQ ID NO: 70 - DNA, 23 bp, Artificial sequence
  Primer
tgatagcattgatgtcacagcct

SEQ ID NO: 71 - DNA, 19 bp, Artificial sequence
  Primer
gccagtgcttgcagaccct

SEQ ID NO: 72 - DNA, 20 bp, Artificial sequence
  Primer
ggctatgacttcggtttggg

SEQ ID NO: 73 - DNA, 19 bp, Artificial sequence
  Primer
gaccacactgcgccaacac

SEQ ID NO: 74 - DNA, 21 bp, Artificial sequence
  Primer
cttctccacaaccctctgcac

SEQ ID NO: 75 - DNA, 20 bp, Artificial sequence
  Primer
gtcgctgttgaagtcagagg

SEQ ID NO: 76 - DNA, 18 bp, Artificial sequence
  Primer
gaaactgtggcgtgatgg

SEQ ID NO: 77 - DNA, 20 bp, Artificial sequence
  Primer
ggttgccaagccttgtctga

SEQ ID NO: 78 - DNA, 19 bp, Artificial sequence
  Primer
agggagttcacatgcgcct

SEQ ID NO: 79 - DNA, 20 bp, Artificial sequence
  Primer
tggagaagggtgaccgactc

SEQ ID NO: 80 - DNA, 20 bp, Artificial sequence
  Primer
tcctcacagggcaatgatcc

SEQ ID NO: 81 - DNA, 20 bp, Artificial sequence
  Primer
ctaccagcgcgcctttaatg

SEQ ID NO: 82 - DNA, 19 bp, Artificial sequence
  Primer
tccggaaggacaggcagtt

SEQ ID NO: 83 - DNA, 23 bp, Artificial sequence
  Primer
ctgcccaggaatgctacagatac

SEQ ID NO: 84 - DNA, 21 bp, Artificial sequence
  Primer
cagggtcaccaagggtacaaa

SEQ ID NO: 85 - DNA, 24 bp, Artificial sequence
  Primer
tgaaagaaaggtagcagagctgaa

SEQ ID NO: 86 - DNA, 18 bp, Artificial sequence
  Primer
ccgcgtgctgaggaatct

SEQ ID NO: 87 - DNA, 19 bp, Artificial sequence
  Primer
aaagcgccaacacgtttga

SEQ ID NO: 88 - DNA, 20 bp, Artificial sequence
  Primer
aggaaccccagcaggaaaag

SEQ ID NO: 89 - DNA, 20 bp, Artificial sequence
  Primer
cttaggagggagagcccacc

SEQ ID NO: 90 - DNA, 20 bp, Artificial sequence
  Primer
ttgttcaggccttcccaaat

SEQ ID NO: 91 - DNA, 20 bp, Artificial sequence
  Primer
taggaaaggactgccgggat

SEQ ID NO: 92 - DNA, 20 bp, Artificial sequence
  Primer
ccgcttcttcacacactgga

SEQ ID NO: 93 - DNA, 20 bp, Artificial sequence
  Primer
tgggcattgacccctacaac

SEQ ID NO: 94 - DNA, 30 bp, Artificial sequence
  Primer
tgggtccctgaagaaccatcaggaagtaga

SEQ ID NO: 95 - DNA, 20 bp, Artificial sequence
  Primer
ggtgaagggagacctggctt

SEQ ID NO: 96 - DNA, 20 bp, Artificial sequence
  Primer
gtgcctcagcaatttctggc